(19) United States
(12) Patent Application Publication
(10) Pub. No.: US 2024/0059712 A1
(43) Pub. Date: Feb. 22, 2024

(54) BIFUNCTIONAL COMPOUNDS AND PHARMACEUTICAL USES THEREOF

(71) Applicant: RISEN (SUZHOU) PHARMA TECH CO., LTD., Suzhou (CN)

(72) Inventors: Jiasheng LV, Suzhou (CN); Xiang JI, Suzhou (CN); Gang WU, Suzhou (CN); Bin ZONG, Suzhou (CN); Qiguo ZHANG, Suzhou (CN); Yanpeng WU, Suzhou (CN); Xiangyang LI, Suzhou (CN); Yuhui CHEN, Suzhou (CN); Yongyue CHEN, Suzhou (CN); Chuanhao HUANG, Suzhou (CN); Xingwu ZHU, Suzhou (CN); Xiaolin HE, Suzhou (CN); Yao LIU, Suzhou (CN); Yuhua ZHANG, Suzhou (CN); Jian GE, Suzhou (CN); Tianlun ZHOU, Suzhou (CN); Xiangsheng YE, Suzhou (CN); Xianqi KONG, Dollard-des-Ormeaux (CA); Dawei CHEN, Suzhou (CN)

(21) Appl. No.: 18/466,473

(22) Filed: Sep. 13, 2023

(30) Foreign Application Priority Data
Mar. 9, 2022 (WO) PCT/CA2023/050308
Sep. 13, 2022 (CN) 202211107827.3
Sep. 13, 2022 (CN) 202211110187.1
Mar. 9, 2023 (CN) 202310218694.5
Jul. 13, 2023 (CN) 202310861549.9

(51) Int. Cl. C07D 519/00

(52) U.S. Cl. CPC C07D 519/00

(57) ABSTRACT

The disclosure relates to bifunctional KRAS-G12D-modulating compounds having the structure W-L-T, where W is a targeting group that binds specifically to KRAS-G12D protein, T is an E3-ligase binding group, and L is absent or is a bivalent linking group that connects W and T together via a covalent linkage. Compounds and pharmaceutical compositions thereof can promote degradation of the KRAS-G12D protein in a cell and are thus useful for treating, inhibiting, and preventing KRAS-G12D-associated diseases, disorders and conditions, including cancers.

### CROSS-REFERENCE TO RELATED APPLICATIONS

This application claims the benefit of priority from Chinese Patent Application No. CN2022111101871, filed Sep. 13, 2022; Chinese Patent Application No. CN2022111078273, filed Sep. 13, 2022; Chinese Patent Application No. CN2023102186945, filed Mar. 9, 2023; International Application No. PCT/CA2023/050308, filed Mar. 9, 2023; U.S. application Ser. No. 18/119,592, filed Mar. 9, 2023; and Chinese Patent Application No. CN2023108615499, filed Jul. 13, 2023, each of which is hereby incorporated by reference in its entirety.

### FIELD

The present disclosure relates to bifunctional KRAS-G12D-modulating compounds, pharmaceutical compositions thereof, and uses thereof for treating, inhibiting and/or preventing KRAS-G12D-associated diseases, disorders and conditions, including cancers, tumors and hyperplastic disorders.

### BACKGROUND

The Kirsten Rat Sarcoma Viral Oncogene Homolog (K-Ras) gene belongs to the Ras family of oncogenes and is one of the most common gene mutations in human cancers. Its encoded protein (KRAS) is part of the RAS/MAPK signal transduction pathway which regulates cell growth and differentiation. KRAS is a small GTPase, a class of enzymes which convert the nucleotide guanosine triphosphate (GTP) into guanosine diphosphate (GDP). It is turned on (activated) by binding to GTP and turned off (inactivated) by converting the GTP to GDP. In this way KRAS acts as a molecular on/off switch. In most cells, KRAS is inactivated. When activated, it can activate several downstream signaling pathways including the MAPK signal transduction pathway, the PI3K signal transduction pathway and the Ral-GEFs signal transduction pathway. These signal transduction pathways play an important role in promoting cell survival, proliferation, and cytokine release, thus affecting tumor occurrence and development.

In human cancers, K-Ras gene mutations occur in nearly 90% of pancreatic cancers, about 30% to 40% of colorectal cancers, about 17% of endometrial cancers, and about 15% to 20% of lung cancers (primarily non-small cell lung cancer, or NSCLC). K-Ras gene mutations also occur in bile duct cancers, cervical cancers, bladder cancers, liver cancers and breast cancers, as well as leukemias. K-RAas gene mutations are thus found at high rates in many different types of cancer.

Many K-Ras gene mutations are missense mutations occurring in codon 12, which results in changing the glycine at position 12 (G12) to another amino acid. Replacements with cysteine, aspartic acid, arginine, and valine (KRAS-G12C, KRAS-G12D, KRAS-G12R, and KRAS-G12V, respectively) are the most common KRAS mutations in patients. In particular, the KRAS-G12D and KRAS-G12V mutations are found in about 90% of pancreatic cancers, and KRAS-G12D is the most common KRAS mutation in colorectal cancer.

Inhibitors of KRAS-G12D have been described (see, for example, International (PCT) Application Publication Nos. WO2021041671 and WO2021106231). In 2021, The U.S. Food and Drug Administration (FDA) approved sotorasib as the first KRAS-G12C blocking drug for the treatment of adult patients with NSCLC. The KRAS-G12C inhibitor adagrasib was also approved by the U.S. FDA in 2022 for treatment of NSCLC. However, existing KRAS inhibitors face significant limitations. One of the biggest obstacles to KRAS inhibitor treatment is the emergence of drug resistance. While the biological basis of acquired drug resistance is not well understood, it has been suggested that several factors may play a role, including cellular heterogeneity in tumors; the activation of wild-type RAS by multiple receptor tyrosine kinases (RTKs) rather than a single RTK; and secondary gene mutations (see, e.g., Liu et al., Cancer Gene Therapy 2022, 29:875-878).

There is a need therefore for new inhibitors that can maintain efficacy and avoid or overcome the difficulties of acquired drug resistance. One method for avoiding or overcoming drug resistance is to promote degradation of the target protein, rather than simply inhibiting its biological activity through direct binding. One such method for enhancing protein degradation is through use of Proteolysis targeting chimeras, or Protacs (see, for example, Angew. Chem. Int. Ed. 2016, 55, 807-810; J. Med. Chem. 2018, 61, 444-452). A Protac is not a traditional enzyme inhibitor but rather acts by inducing intracellular protein hydrolysis (proteolysis). Such targeted protein degradation has emerged as a new paradigm to manipulate cellular proteostasis. In general, proteolysis targeting chimeras (Protacs) are bifunctional small molecules composed of two active domains and optionally a linker. One of the two active domains binds to E3 ubiquitin ligase, and the other to a target protein of interest. A Protac can thus remove a target protein of interest by binding to the target protein and recruiting an E3 ligase thereto, which catalyzes ubiquitination and leads to subsequent degradation of the target protein. Compared to traditional inhibitors that may need to inhibit enzymatic activity of a target protein, Protacs need only to bind specifically to the target protein to be effective.

There is a need for KRAS inhibitors effective for the treatment or prevention of KRAS-related diseases or disorders, including those associated with the KRAS-G12D mutation.

### SUMMARY

The present disclosure relates to bifunctional compounds and compositions comprising the compounds that inhibit the KRAS protein. Specifically, the disclosure provides proteolysis targeting chimera (Protac) compounds that bind to both the target protein of interest (e.g., KRAS-G12D) and to an E3 ligase. By binding to both molecules, these compounds can recruit the E3 ligase to the target protein of interest, promoting its ubiquitination and subsequent degradation.

The present disclosure also relates to the use of such compounds and compositions for the treatment and/or prevention of diseases, disorders and conditions mediated, in whole or in part, by KRAS, e.g., by KRAS-G12D. KRAS inhibitors have been linked to the treatment of many hyperplastic and hyperproliferative diseases and disorders, including cancers and tumors. In particular embodiments, the KRAS-G12D inhibitor compounds and compositions described herein can act to modulate degradation of KRAS-G12D and are thus useful as therapeutic or prophylactic agents when such degradation is desirable, e.g., for tumors and cancers associated with the K-Ras-G12D mutation and/or the KRAS-G12D mutant protein.

In a first broad aspect, there are provided compounds of Formula (I) and pharmaceutically acceptable salts, esters, hydrates, solvates, or stereoisomers thereof:

W-L-T (I)

where: W is a targeting group that binds specifically to a target protein of interest; T is an E3-ligase binding group; and L is absent or is a bivalent linking group that connects W and T together via a covalent linkage.

In certain embodiments of compounds of Formula (I), the target protein of interest is KRAS, e.g., KRAS-G12D. In such embodiments, W is a KRAS, e.g., KRAS-G12D, targeting group, i.e., a targeting group that binds specifically to the KRAS-G12D protein.

In certain embodiments of compounds of Formula (I), the targeting group W is a KRAS-G12D targeting group having the structure of Formula (Ia) or Formula (Ib):

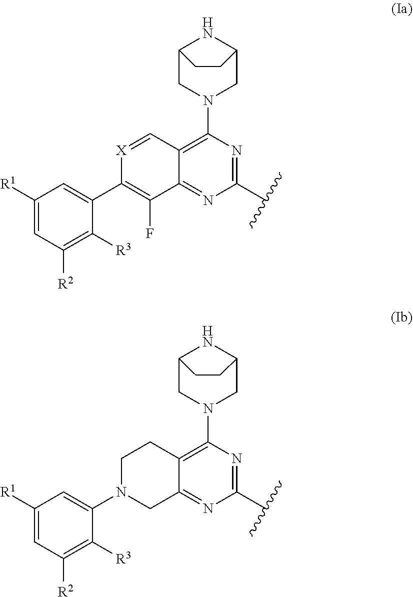

where:
X is a nitrogen (N) or an unsubstituted or substituted carbon (C);
R¹ is unsubstituted or substituted hydroxyl, amino, or thio group; and
R² and R³ are selected independently from hydrogen (H), halogen (X), halogen substituted methyl (—CH₂X₁, —CHX₂, or —CX₃), or R² and R³, together with the phenyl-ring structure to which they are attached, form an unsubstituted or substituted benzo-fused ring.

In certain embodiments of KRAS-G12D targeting groups of Formulae (Ia) and (Ib), the unsubstituted or substituted benzo-fused ring is a naphthyl ring system. In some such embodiments, the benzo-fused ring is further substituted with one or more substituents. In some such embodiments, the benzo-fused ring is further substituted with one or more substituents selected from halogen, hydroxyl, amino, halomethyl, C₁-C₂ alkyl, and C₂ to C₄ alkynyl group.

In certain embodiments of KRAS-G12D targeting groups of Formulae (Ia) and (Ib), the benzo-fused ring is substituted with one or more substituents selected from halogen, hydroxyl, amino, halomethyl, C₁-C₂ alkyl, and C₂ to C₄ alkynyl group.

In certain embodiments of KRAS-G12D targeting groups of Formulae (Ia) and (Ib), X is CH, C—F, C—Cl, C—CH₃, C—C₂H₅, or C—C₃H₇.

In certain embodiments of KRAS-G12D targeting groups of Formulae (Ia) and (Ib), X is CH, C—F, C—Cl, C—CH₃, C—C₂H₅, C—C₃H₇, or N.

In certain embodiments of KRAS-G12D targeting groups of Formulae (Ia) and (Ib), X is an unsubstituted or substituted carbon (C).

In certain embodiments of KRAS-G12D targeting groups of Formulae (Ia) and (Ib), X is N.

In certain embodiments of KRAS-G12D targeting groups of Formulae (Ia) and (Ib),

In certain embodiments of compounds of Formula (I), the targeting group W is a KRAS-G12D targeting group having the structure of Formula (Ia).

In certain embodiments of compounds of Formula (I), the targeting group W is a KRAS-G12D targeting group having the structure of Formula (Ib).

In certain embodiments of compounds of Formula (I), the targeting group W is a KRAS-G12D targeting group having the structure of Formula (Ia), wherein X is N.

In certain embodiments of compounds of Formula (I), the targeting group W is a KRAS-G12D targeting group having the structure of Formula (Ia), wherein R¹ is —OH.

In certain embodiments of compounds of Formula (I), the targeting group W is a KRAS-G12D targeting group having the structure of Formula (Ia), wherein R² and R³, together with the phenyl-ring structure to which they are attached, form a substituted benzo-fused ring.

In certain embodiments of compounds of Formula (I), the targeting group W is a KRAS-G12D targeting group having the structure of Formula (Ia), wherein X is N; R¹ is —OH; and/or R² and R³, together with the phenyl-ring structure to which they are attached, form a substituted benzo-fused ring.

In certain embodiments of compounds of Formula (I), the targeting group W comprises a fragment having the structure:

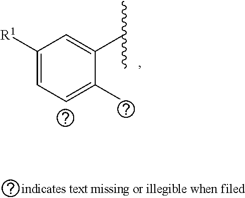

wherein R¹, R² and R³ are as defined above.

In certain embodiments of KRAS-G12D targeting groups of Formulae (Ia) and (Ib), the left end fragment

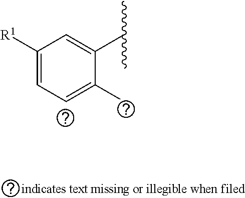

is selected from the following:

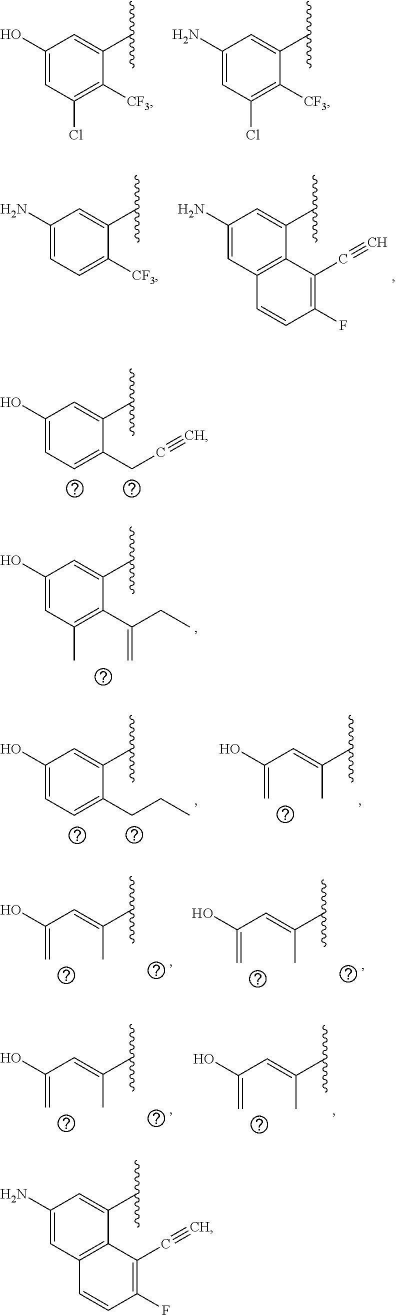

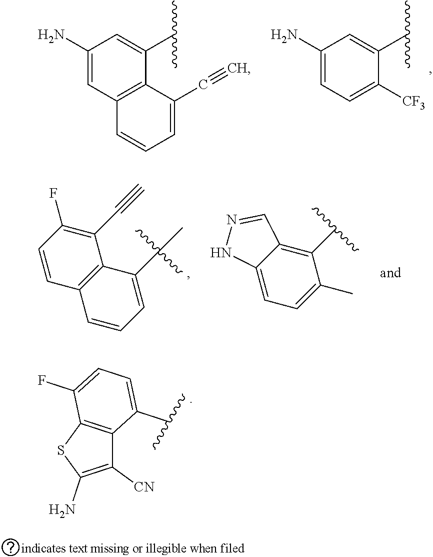

In certain embodiments of compounds of Formula (I), the E3 ligase binding group (T) comprises a ligand of an E3 ligase (i.e., is a ligand group).

In certain embodiments of compounds of Formula (I), the E3 ligase binding group (T) binds specifically to an E3 ligase which is VHL (Von Hippel-Lindau), CRBN (Cereblon), MDM2, c-IAP1, AhR, Nimbolide, CCW16, KB02, KEAP1, beta-TrCP1, DCAF15, DCAF16, RNF114, or another E3 ligase. In some embodiments, the E3 ligase binding group (T) binds specifically to an E3 ligase which is VHL (Von Hippel-Lindau), CRBN (Cereblon), MDM2, c-IAP1, AhR, Nimbolide, CCW16, KB02 or KEAP1. In some embodiments, the E3 ligase binding group (T) binds specifically to an E3 ligase which is VHL. In some embodiments, the E3 ligase binding group (T) binds specifically to an E3 ligase which is CRBN.

In some embodiments of compounds of Formula (I), the E3 ligase binding group (T) has the structure of Formula (IIIa) or (IIIb):

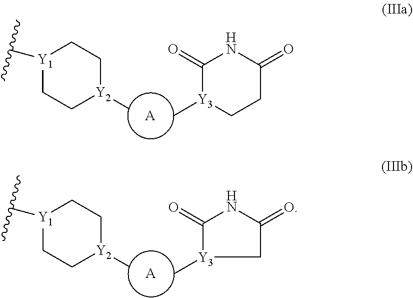

In certain embodiments of Formulae (IIIa) and (IIIb), A has the structure of

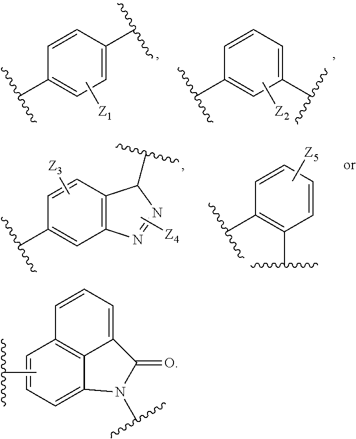

In certain embodiments of Formulae (IIIa) and (IIIb), Z₁, Z₂, and Z₅ are independently alkyl (e.g., substituted or unsubstituted C₁-C₂₀ alkyl, substituted or unsubstituted C₁-C₆ alkyl, substituted or unsubstituted C₁, C₂, C₃, C₄, C₅ or C₆ alkyl), halogen (X), oxygen (O) or absent (i.e., do not exist).

In certain embodiments of compounds of Formulae (IIIa) and (IIIb), Z₃ and Z₄ are independently hydrogen (H), halogen (X), alkyl (e.g., substituted or unsubstituted C₁-C₂₀ alkyl, substituted or unsubstituted C₁-C₆ alkyl, substituted or unsubstituted C₁, C₂, C₃, C₄, C₅ or C₆ alkyl) or absent (i.e., do not exist).

In certain embodiments of compounds of Formulae (IIIa) and (IIIb), Z₁, Z₂, Z₃, Z₄ and/or Z₅ can be substituted at any site on a benzene ring or a five-membered ring, e.g., on a ring in A.

In certain embodiments of compounds of Formulae (IIIa) and (IIIb), Z₃ and Z₄ are independently hydrogen (H), halogen (X), substituted or unsubstituted C₁-C₆ alkyl (e.g., C₁, C₂, C₃, C₄, C₅ or C₆ alkyl) or absent (i.e., do not exist). In some such embodiments, Z₃ and Z₄ are independently substituted or unsubstituted C₁-C₆ alkyl (e.g., C₁, C₂, C₃, C₄, C₅ or C₆ alkyl) or absent. In some such embodiments, Z₃ and Z₄ are independently substituted or unsubstituted C₁-C₆ alkyl (e.g., C₁, C₂, C₃, C₄, C₅ or C₆ alkyl) or absent. In some such embodiments, Z₃ and Z₄ are independently substituted or unsubstituted CH₃, halogen or absent. In some such embodiments, Z₃ and Z₄ are independently CH₃, F or absent. In some such embodiments, Z₃ and Z₄ are independently CH₃ or absent. In some such embodiments, Z₃ and Z₄ are independently CH₃ or F.

In certain embodiments of compounds of Formulae (IIIa) and (IIIb), Y₁, Y₂, and Y₃ are independently carbon (C) or nitrogen (N).

In certain embodiments of compounds of Formulae (IIIa) and (IIIb), Y₁ is nitrogen; Y₃ is nitrogen; or both Y₁ and Y₃ are nitrogen.

In certain embodiments of compounds of Formulae (IIIa) and (IIIb), Y₂ is carbon.

In certain embodiments of compounds of Formulae (IIIa) and (IIIb), Y₁ is nitrogen; Y₂ is carbon; and Y₃ is nitrogen.

In certain embodiments of compounds of Formulae (IIIa) and (IIIb), Z₁, Z₂ and Z₅ are independently substituted or unsubstituted C₁-C₆ alkyl (e.g., C₁, C₂, C₃, C₄, C₅ or C₆ alkyl) or absent. In some such embodiments, Z₁, Z₂ and Z₅ are independently CH₃ or absent. In some such embodiments, Z₁, Z₂ and Z₅ are independently CH₃ or F.

In certain embodiments of compounds of Formulae (IIIa) and (IIIb), Z₁, Z₂ and Z₅ are independently CH₃, halogen or absent. In some such embodiments, Z₁, Z₂ and Z₅ are independently CH₃, F or absent.

In certain embodiments of compounds of Formulae (IIIa) and (IIIb), the halogen is fluorine (F), chlorine (Cl) or bromine (Br). In some such embodiments, the halogen is F.

In certain embodiments of compounds of Formulae (IIIa) and (IIIb), A is

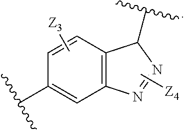

In some such embodiments, Z₃ is F or absent and Z₄ is alkyl. In some such embodiments, Z₄ is C₁-C₆ alkyl. In some such embodiments, Z₄ is CH₃. In some such embodiments, Z₃ is F. In some such embodiments, Z₃ is absent. In one embodiment, Z₃ is absent and Z₄ is CH₃. In another embodiments, Z₃ is F and Z₄ is CH₃.

In some embodiments of compounds of the disclosure, the E3 ligase binding group (T) binds specifically to VHL and has the following structure:

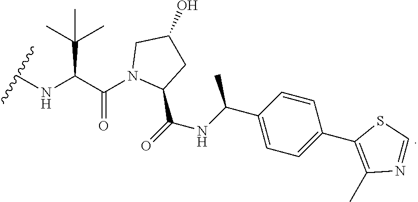 [SMILES: *N[C@H](C(=O)N1C[C@H](O)C[C@H]1C(=O)N[C@@H](C)c1ccc(-c2scnc2C)cc1)C(C)(C)C]

In some embodiments of compounds of the disclosure, the E3 ligase binding group (T) binds specifically to VHL and has the following structure:

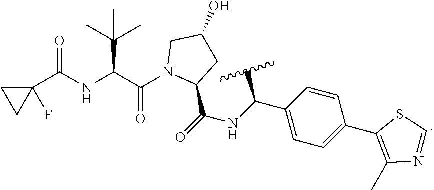 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, the E3 ligase binding group (T) binds specifically to CRBN and has the following structure:

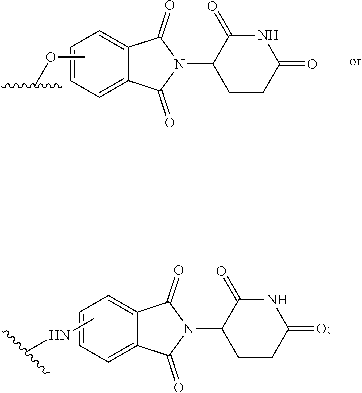

wherein —O— and/or —NH— are connected at any position of the phenyl ring where a substitution is possible.

In certain embodiments of compounds of Formula (I), the E3 ligase binding group (T) has a structure which is:

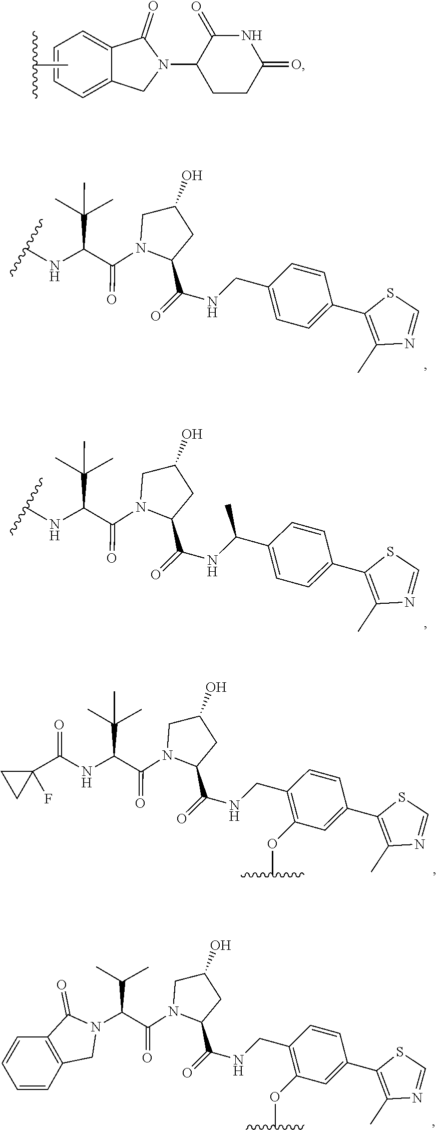

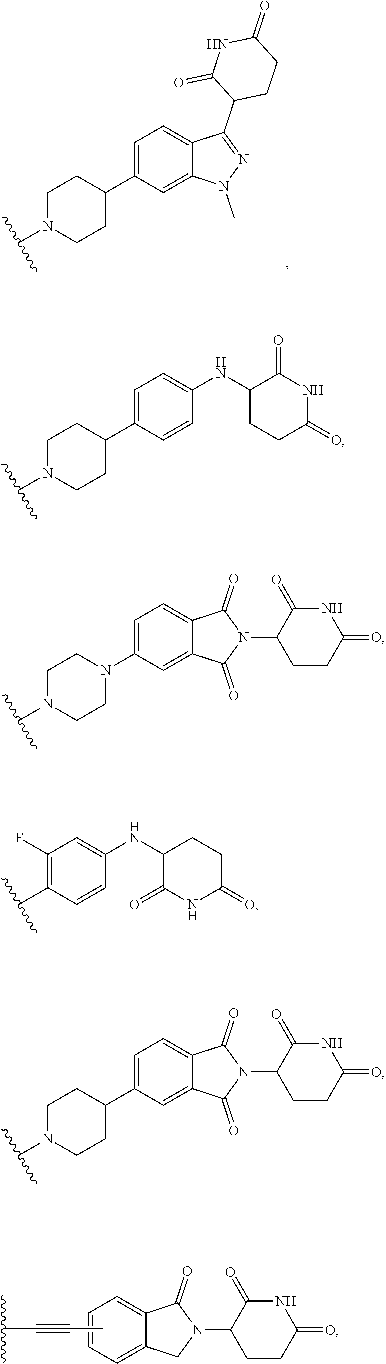

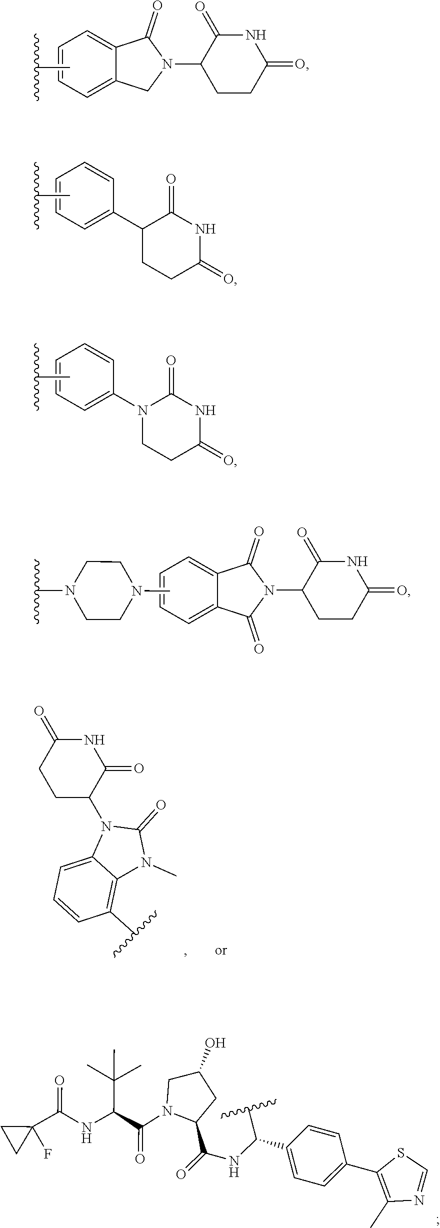

wherein the connecting point is any position of the phenyl ring where a substitution is possible, and the group is R-configuration, S-configuration, or a mixture of R- and S-configurations.

In certain embodiments of compounds of Formula (I), the E3 ligase binding group (T) has a structure which is:

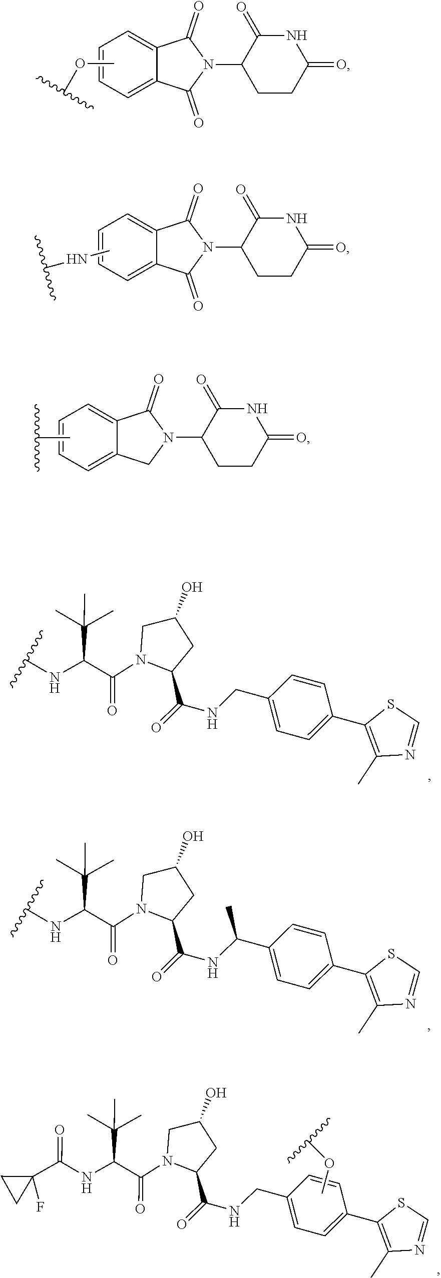

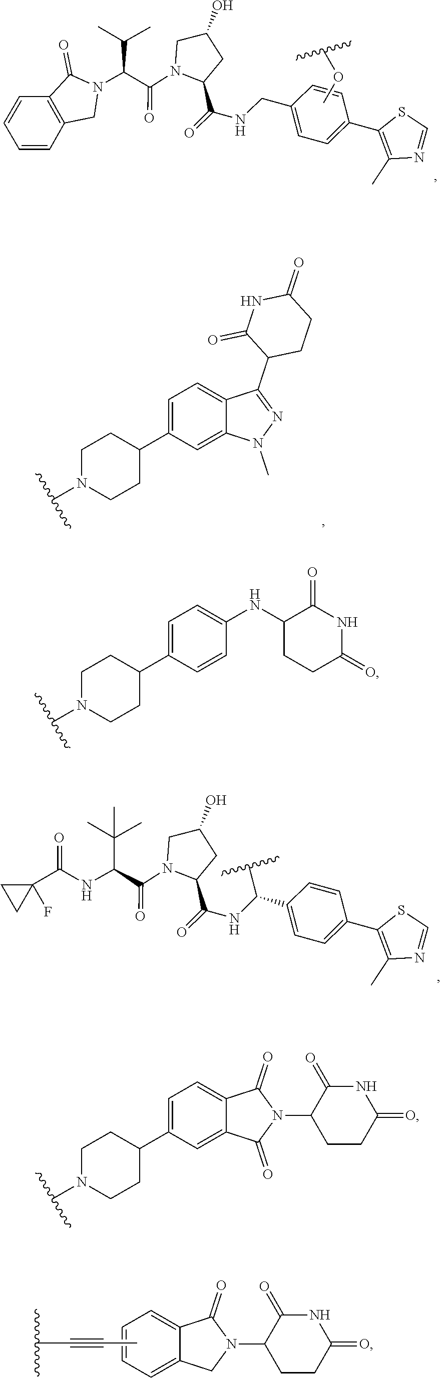

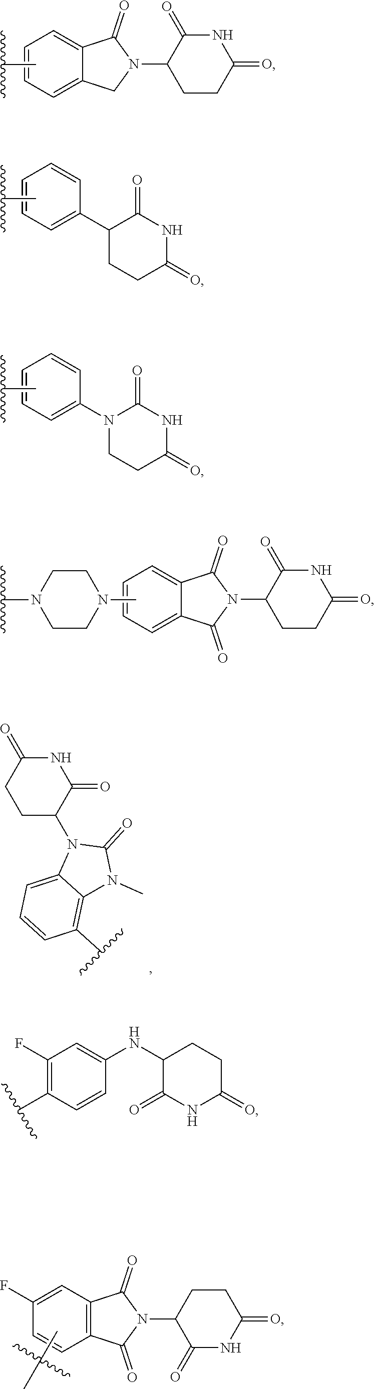

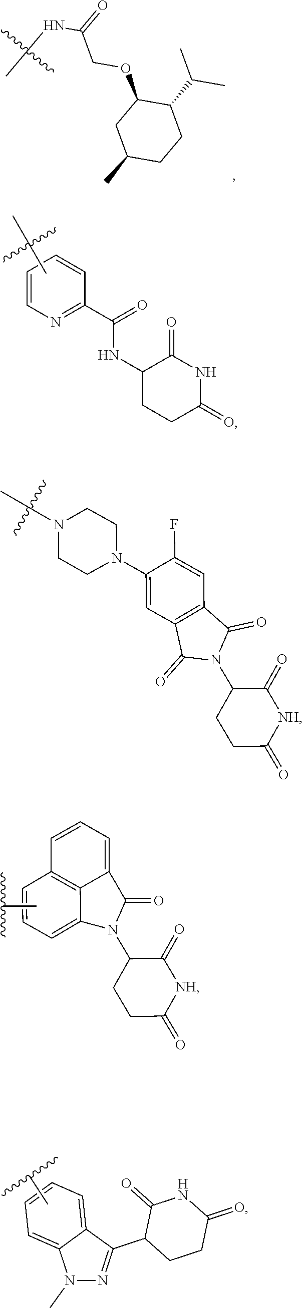

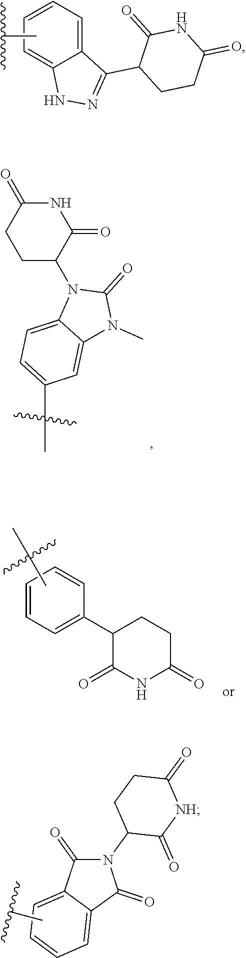

where the connecting point is any position of the phenyl ring capable of substitution.

In certain embodiments of compounds of Formula (I), the E3 ligase binding group (T) has a structure which is:

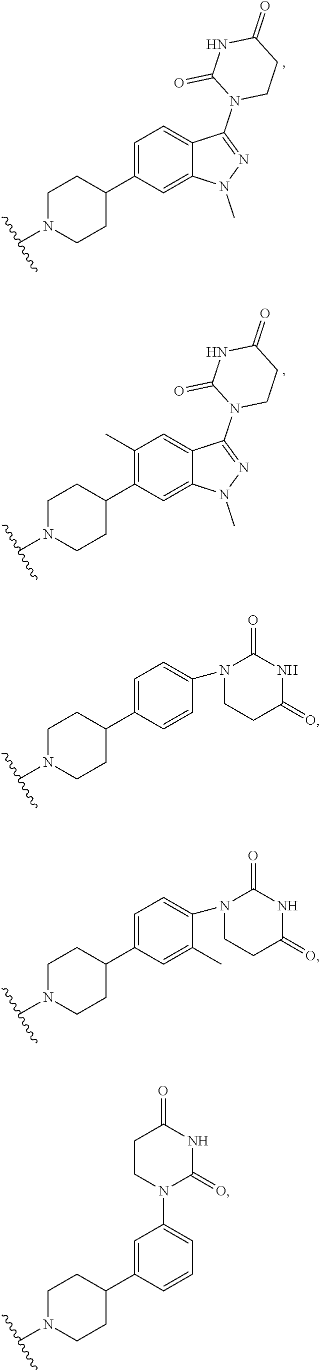 [SMILES: *N1CCC(c2ccc3c(N4CCC(=O)NC4=O)nn(C)c3c2)CC1 ; *N1CCC(c2cc3c(cc2C)c(N2CCC(=O)NC2=O)nn3C)CC1 ; *N1CCC(c2ccc(N3CCC(=O)NC3=O)cc2)CC1 ; *N1CCC(c2ccc(N3CCC(=O)NC3=O)c(C)c2)CC1 ; *N1CCC(c2cccc(N3CCC(=O)NC3=O)c2)CC1]

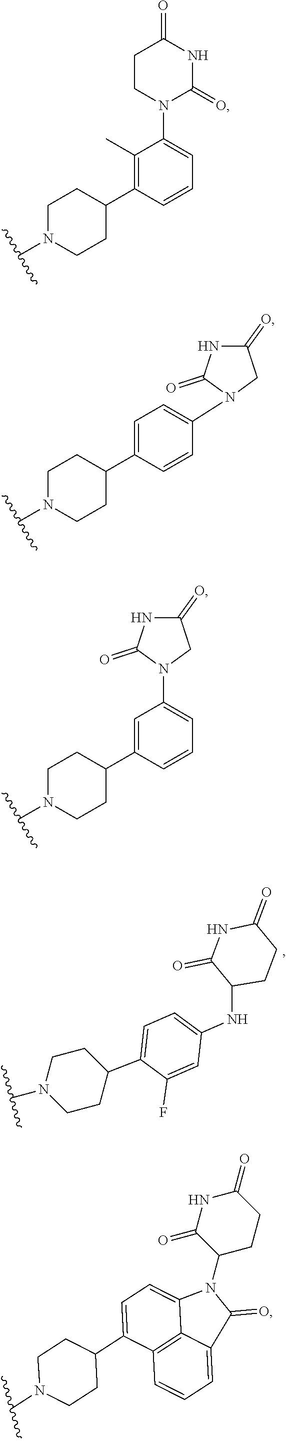 [SMILES: *N1CCC(c2cccc(N3CCC(=O)NC3=O)c2C)CC1 ; *N1CCC(c2ccc(N3CC(=O)NC3=O)cc2)CC1 ; *N1CCC(c2cccc(N3CC(=O)NC3=O)c2)CC1 ; *N1CCC(c2ccc(NC3CCC(=O)NC3=O)cc2F)CC1 ; *N1CCC(c2ccc3c4c(cccc24)C(=O)N3C2CCC(=O)NC2=O)CC1]

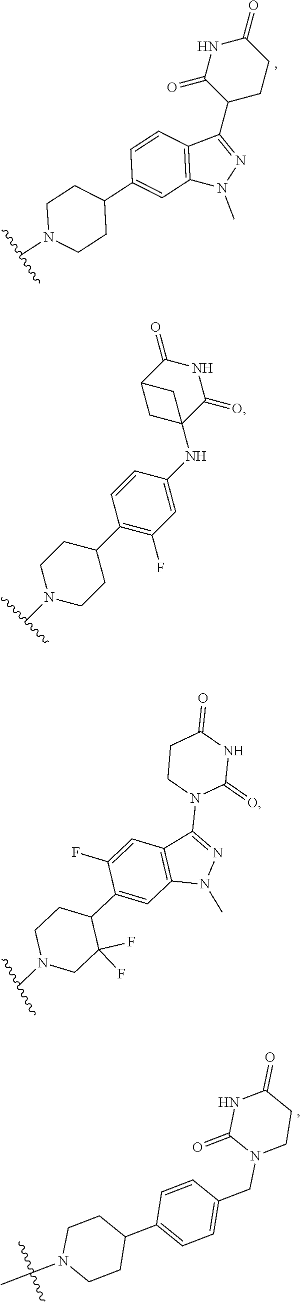 [SMILES: *N1CCC(c2ccc3c(C4CCC(=O)NC4=O)nn(C)c3c2)CC1 ; *N1CCC(c2ccc(NC34CC(C3)C(=O)NC4=O)cc2F)CC1 ; *N1CCC(c2cc3c(cc2F)c(N2CCC(=O)NC2=O)nn3C)C(F)(F)C1 ; *N1CCC(c2ccc(CN3CCC(=O)NC3=O)cc2)CC1]

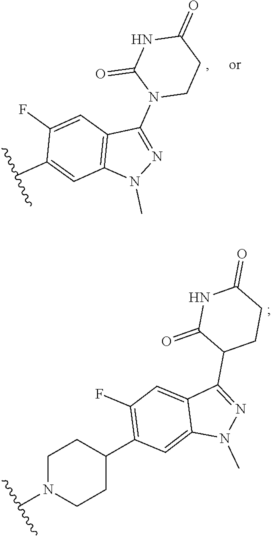 [SMILES: *c1cc2c(cc1F)c(N1CCC(=O)NC1=O)nn2C ; *N1CCC(c2cc3c(cc2F)c(C2CCC(=O)NC2=O)nn3C)CC1]

where the connecting point is any position of the phenyl ring capable of substitution.

In certain embodiments of compounds of Formula (I), L is absent, and the compound has the formula W-T. In such embodiments, the targeting group (W) is covalently connected to the E3 ligase binding group (T) directly.

In certain embodiments of compounds of Formula (I), the bivalent linking group L is present and has the structure L¹-L²-L³, wherein L¹, L² and L³ are all present at the same time, or, optionally, one or two of L¹, L^{z} and L³ are present. In some such embodiments, the compound has the structure W-L¹-L²-L³-T. When one or two of L¹, L² and L³ are present, the compound may have the structure W-L¹-L²-T, W-L¹-L³-T, W-L²-L³-T, W-L¹-T, W-L²-T, or W-L³-T.

In certain embodiments of compounds of Formula (I), the bivalent linking group L has the structure L¹-L²-L³, wherein L¹, L² and L³ are all present at the same time. In such embodiments, the compound has the structure W-L¹-L²-L³-T, and L¹, L² and L³ are as defined above and below.

In certain embodiments of compounds of Formula (I), L¹, L² and L³ are independently selected from substituted or unsubstituted bivalent alkyl, alkloxyl, oxyalkyl, cycloalkyl, heterocycloalkyl, acylalkyl, alkylacyl, carbonylalkyl, alkylcarbonyl, amidoalkyl, alkylamide, aryl, and oligopeptide group having bivalent connecting site.

In some such embodiments, alkyl group includes saturated hydrocarbon group, unsaturated hydrocarbon group, aromatic hydrocarbon group, oxygen hydrocarbon group, nitrogen hydrocarbon group, sulfur hydrocarbon group, phosphorus hydrocarbon group or mixed heterohydrocarbon group with different heteroatoms, wherein the chain length of the hydrocarbon group or the heterohydrocarbon group is from 1 to 20 atoms, and, when alkyl group is heterohydrocarbon group, the heterohydrocarbon group contains from 1 to 5 heteroatoms.

In some such embodiments, the heterocycle in the heterocycloalkyl group or the heterocyclic hydrocarbon group includes substituted or unsubstituted single ring, spiral ring, fused ring, or bridged ring. In some such embodiments, the valence of a heteroatom is satisfied by optional attachment or bonded to H, O, N, or another substituent.

In certain embodiments of compounds of Formula (I), the bivalent linking group L contains only L¹ and has the structure L¹. In such embodiments, the compound has the structure W-L¹-T and L¹ is as defined above and below.

In certain embodiments of compounds of Formula (I), the bivalent linking group L contains L¹ and L², and has the structure L¹-L². In such embodiments, the compound has the structure W-L¹-L²-T and L¹ and L² are as defined above and below.

In certain embodiments of compounds of Formula (I), the bivalent linking group L contains L² and L³, and has the structure L²-L³. In such embodiments, the compound has the structure W-L²-L³-T and L² and L³ are as defined above and below.

In certain embodiments of compounds of Formula (I), L¹ is absent.

In certain embodiments of compounds of Formula (I), L¹ is —O— or —NH—.

In certain embodiments of compounds of Formula (I), L¹ has the structure shown in any one of Formulae (IIa) to (IIk):

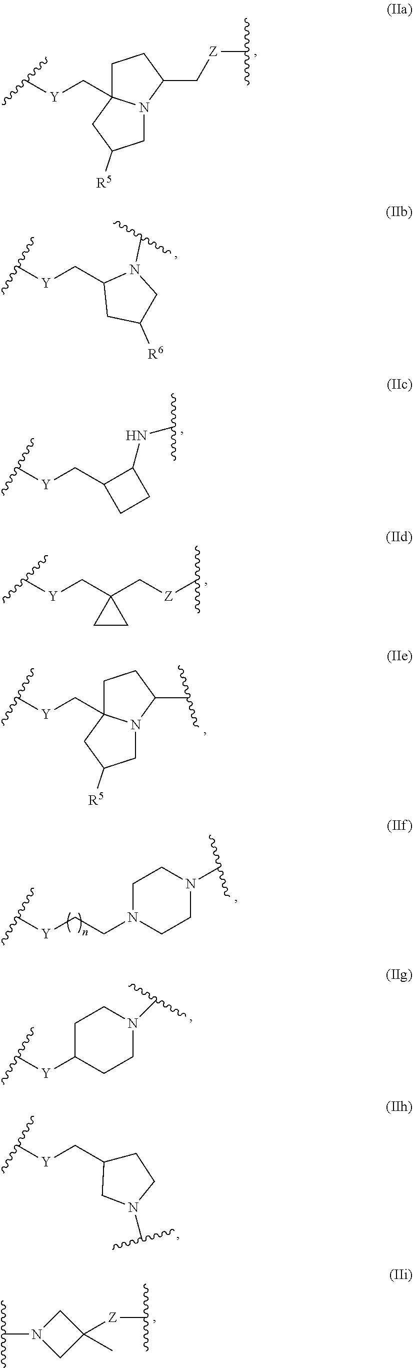

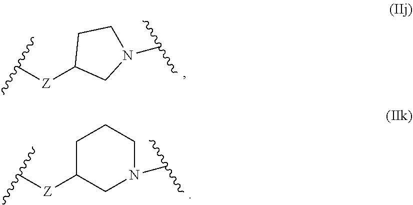

where:
Y and Z are independently oxygen (O), nitrogen (NH), or sulfur (S); or, Y is O, NH or S and Z is a six-membered heterocyclic group;
n is 0-20 (i.e., 0, 1, 2, 3, 4, 5, 6, 7, 8, 9, 10, 11, 12, 13, 14, 15, 16, 17, 18, 19 or 20);
R⁵ and R⁶ are independently hydrogen (H), halogen (such as F, Cl, Br, or I), hydroxy (OH), alkoxy, amino, or substituted amino (such as alkylamino);
wherein, when a chiral center exists, the chiral center has a configuration of R, S, or a mixture of R and S.

In some such embodiments, Z is a six-membered heterocyclic group.

In some such embodiments, n is 0-5 (i.e., n is 0, 1, 2, 3, 4, or 5). In one embodiment, n is 2.

In certain embodiments of compounds of Formula (I), L¹ is:

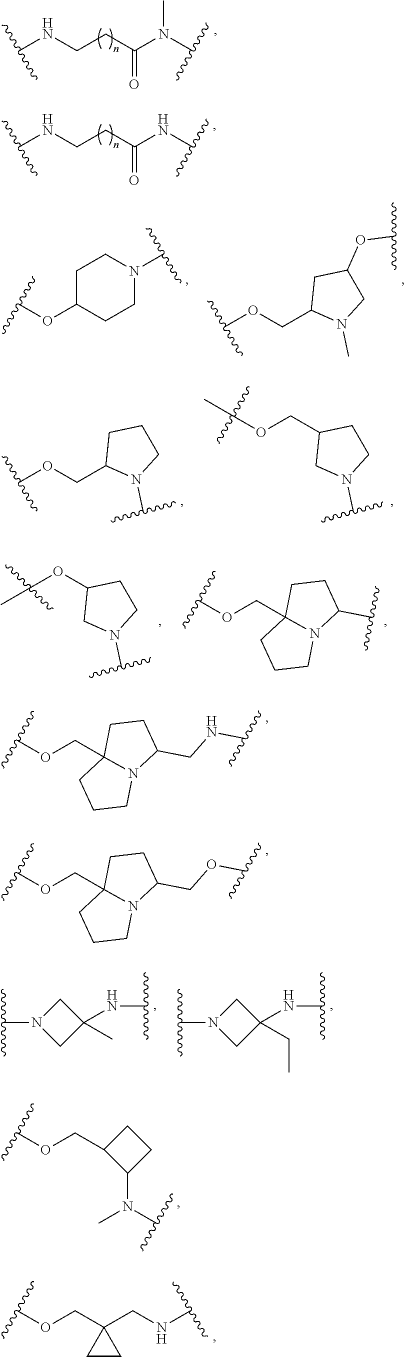

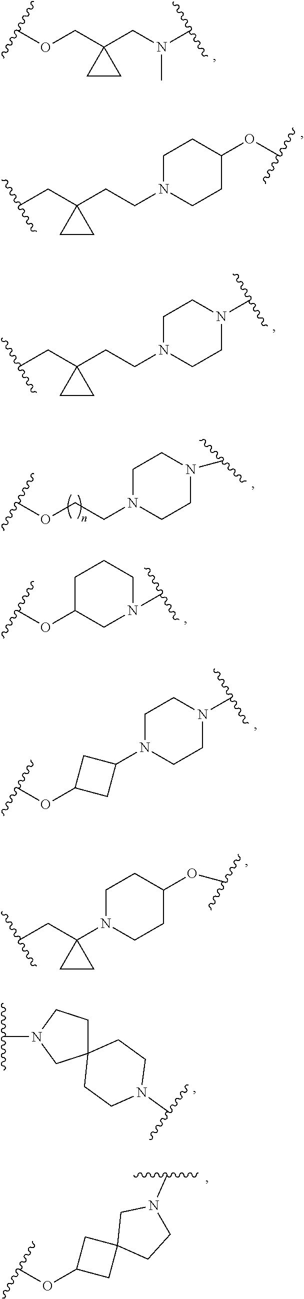

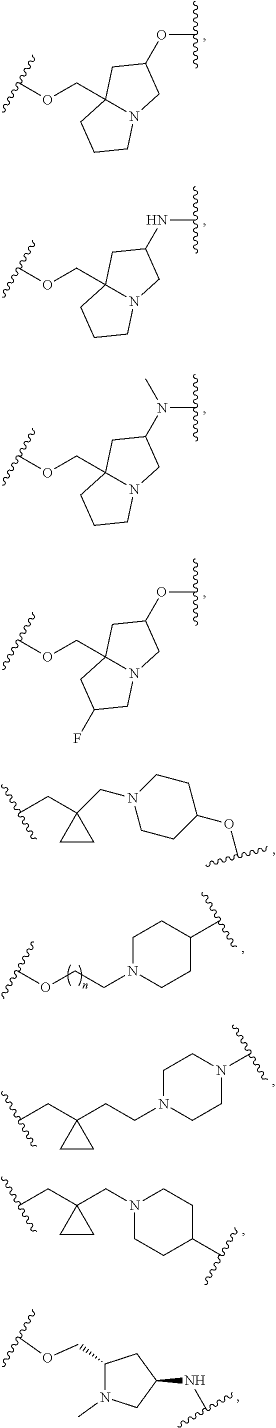

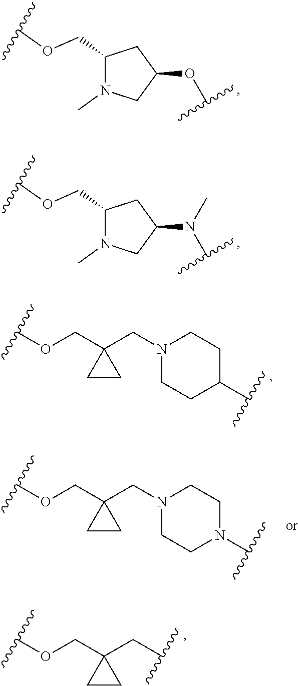

wherein n is an integer from 0 to 20. In some such embodiments, n is an integer from 0 to 5. In some such embodiments, n is 1 or 2. In alternative embodiments, L¹ is absent.

In certain embodiments of compounds of Formula (I), L² and L³ are absent.

In certain embodiments of compounds of Formula (I), L² and L³ are independently selected from —O— or —NH—.

In certain embodiments of compounds of Formula (I), L² and L³ are independently selected from:

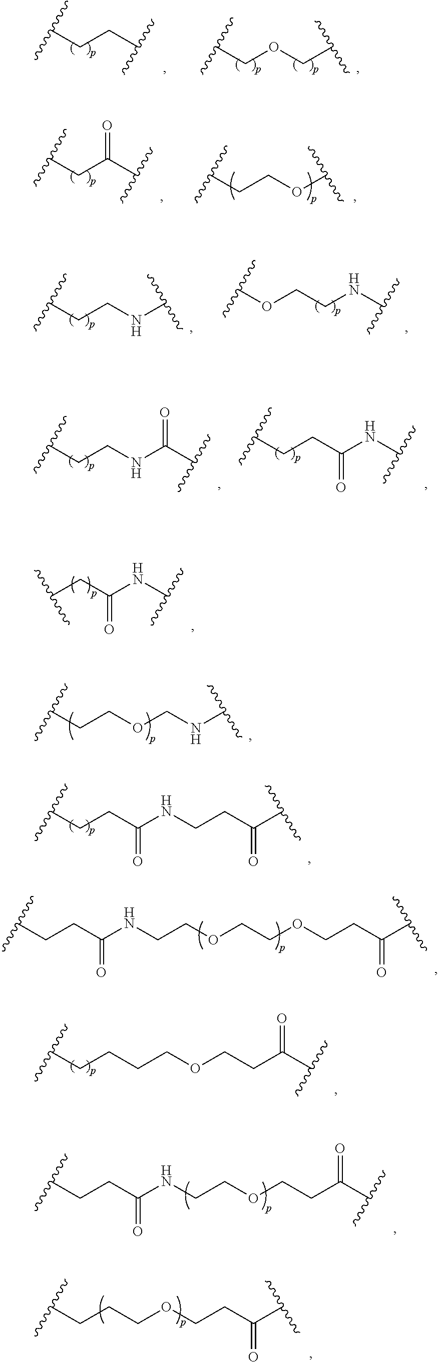

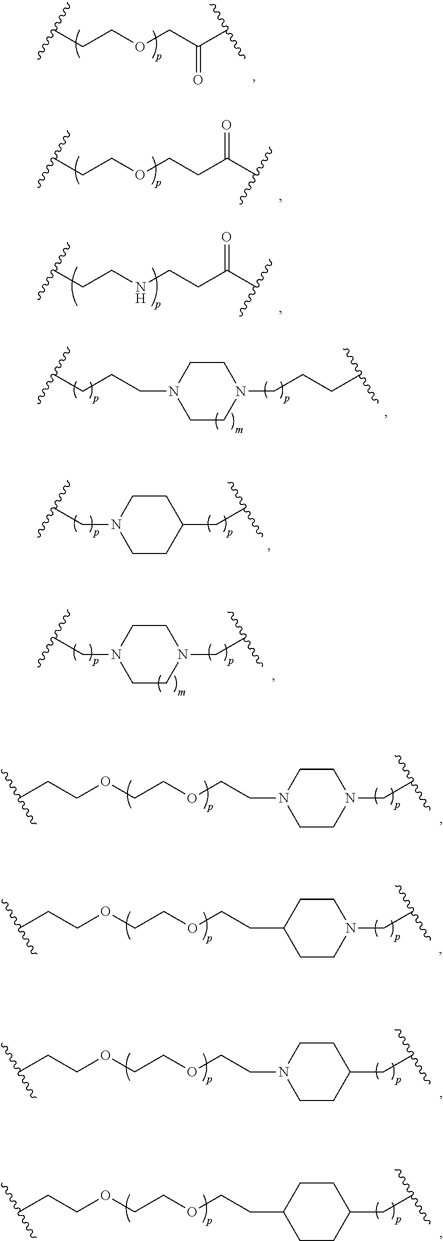

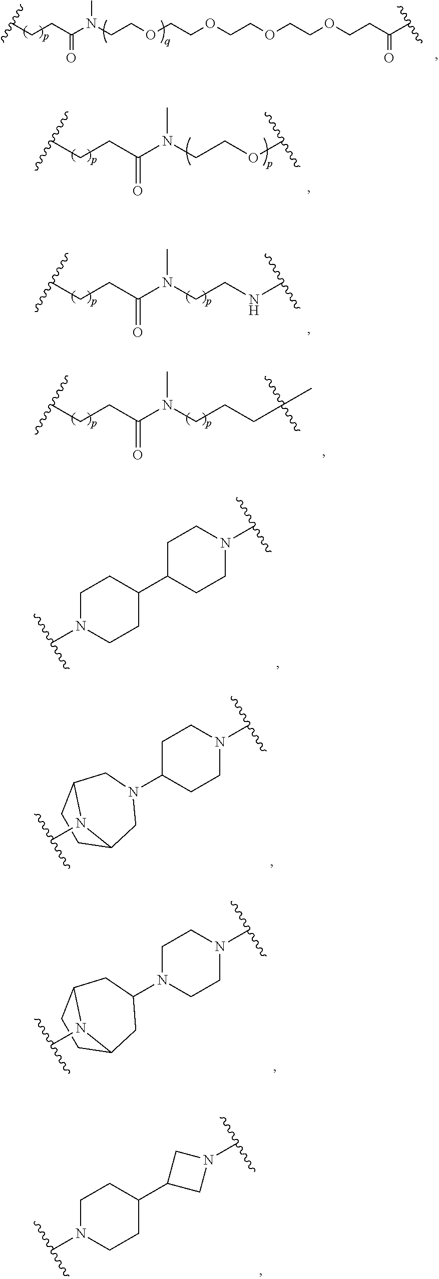

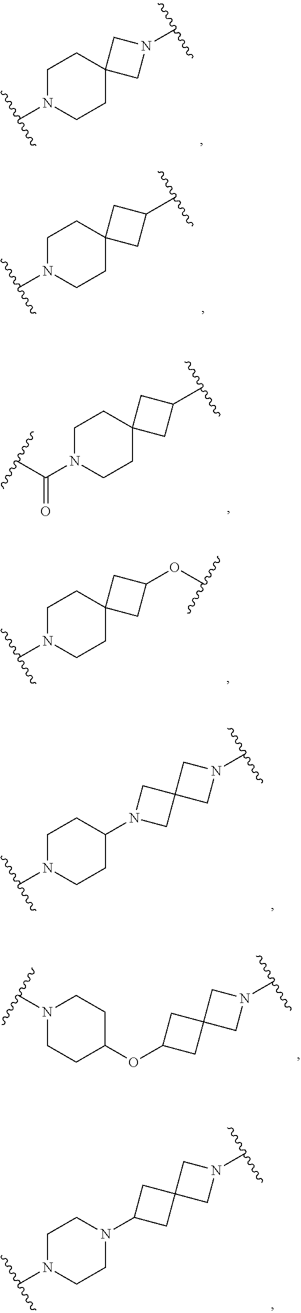

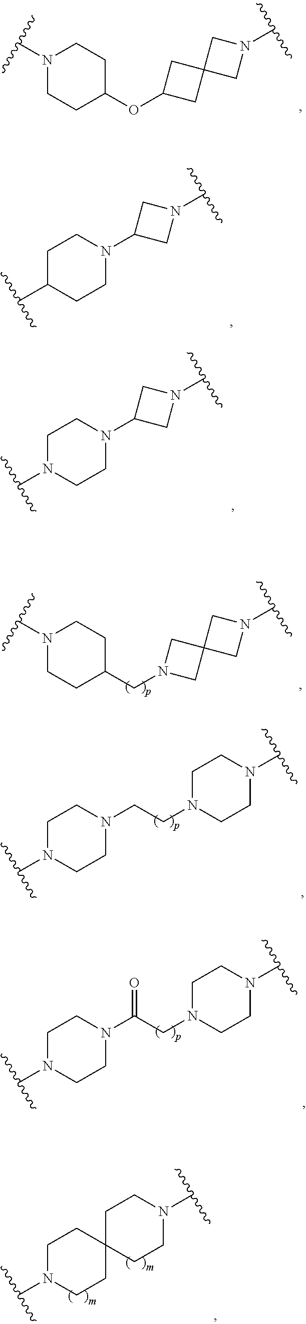

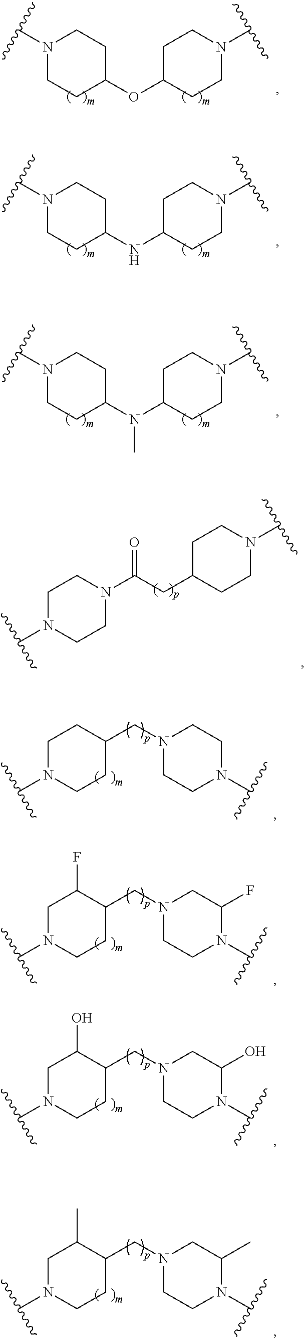

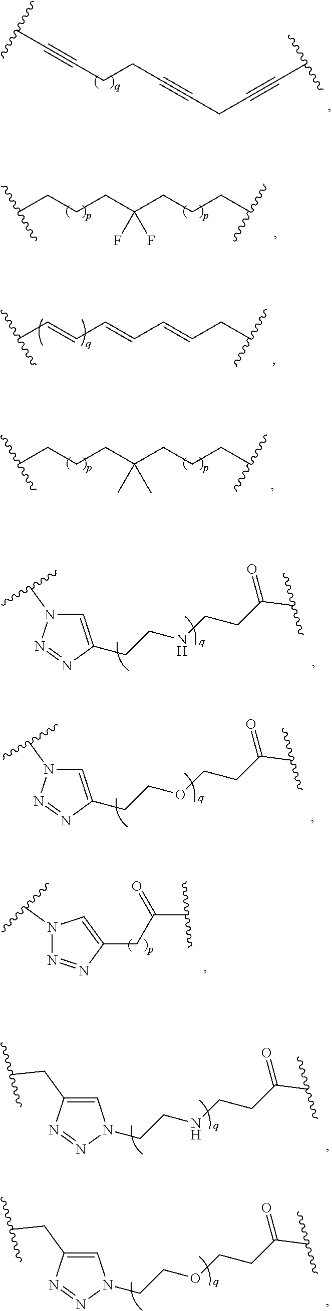

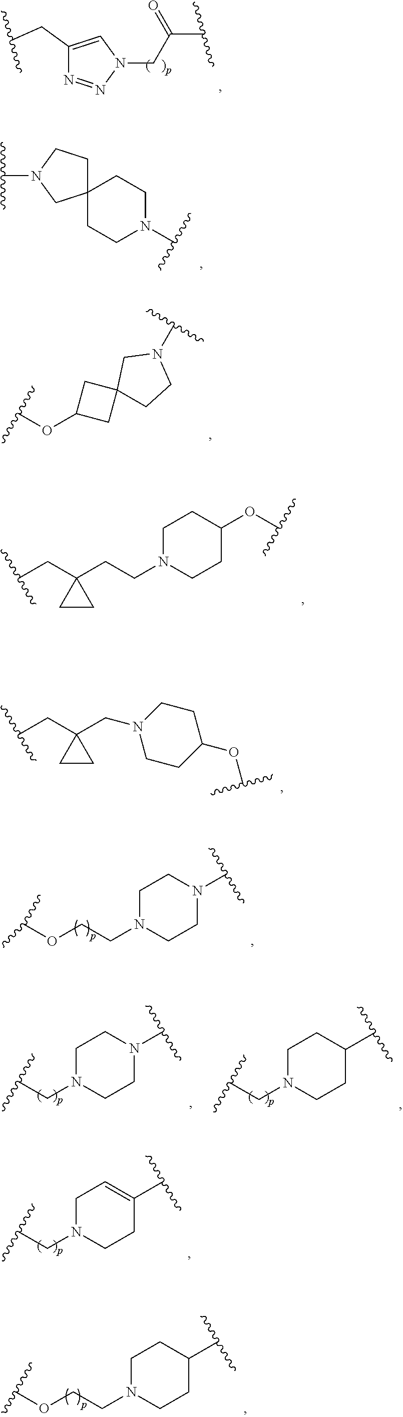

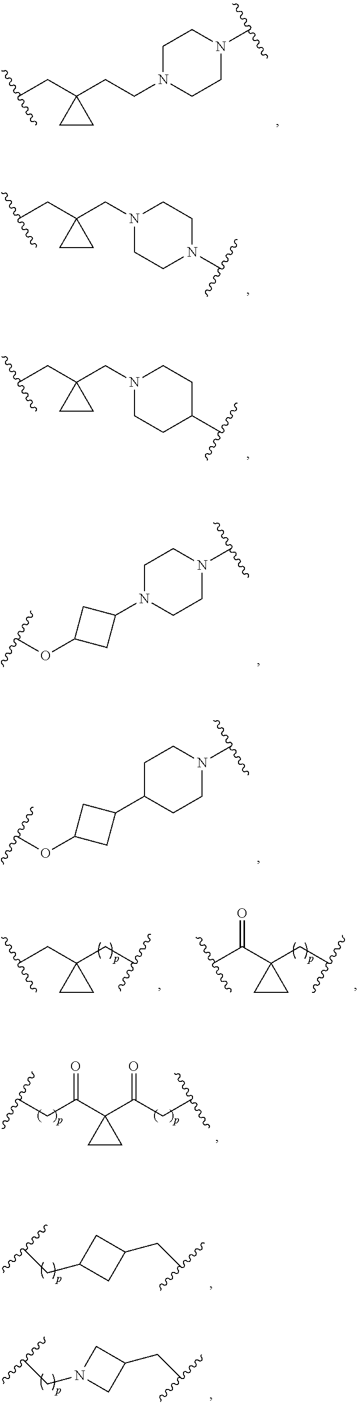

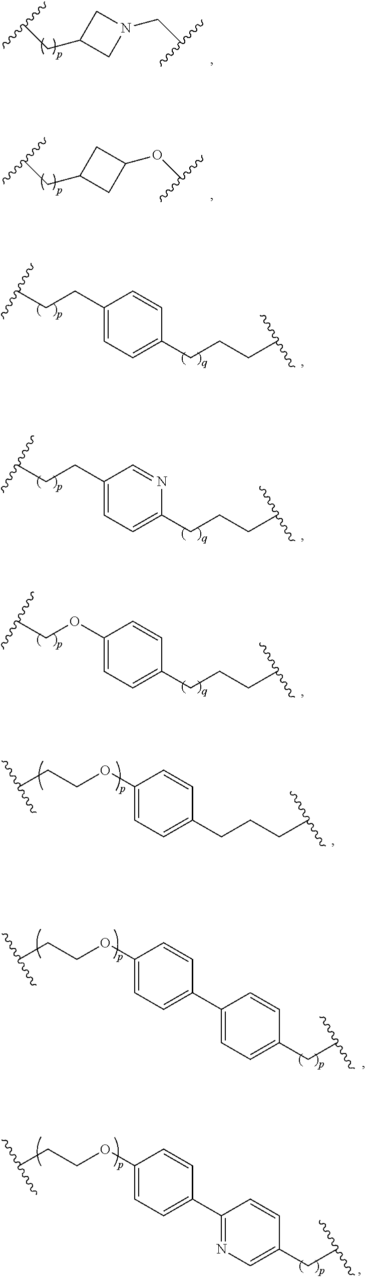

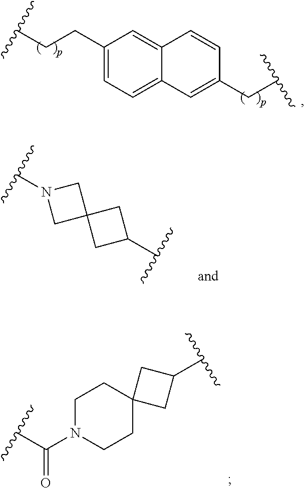

where: p is an integer from 0 to 20 (i.e., p is 0, 1, 2, 3, 4, 5, 6, 7, 8, 9, 10, 11, 12, 13, 14, 15, 16, 17, 18, 19, or 20); m is an integer from 0 to 5 (i.e., m is 0, 1 2, 3, 4 or 5); and q is an integer from 0 to 10 (i.e., q is 0, 1, 2, 3, 4, 5, 6, 7, 8, 9 or 10). In some such embodiments, p is 0-10. In some such embodiments, q is 0-5. In some such embodiments, p is 0-10 and q is 0-5.

In certain embodiments of compounds of Formula (I), one of L² and L³ is absent (and L¹ may be absent or present).

In certain embodiments of compounds of Formula (I), L² and L³ together form a structure selected from:

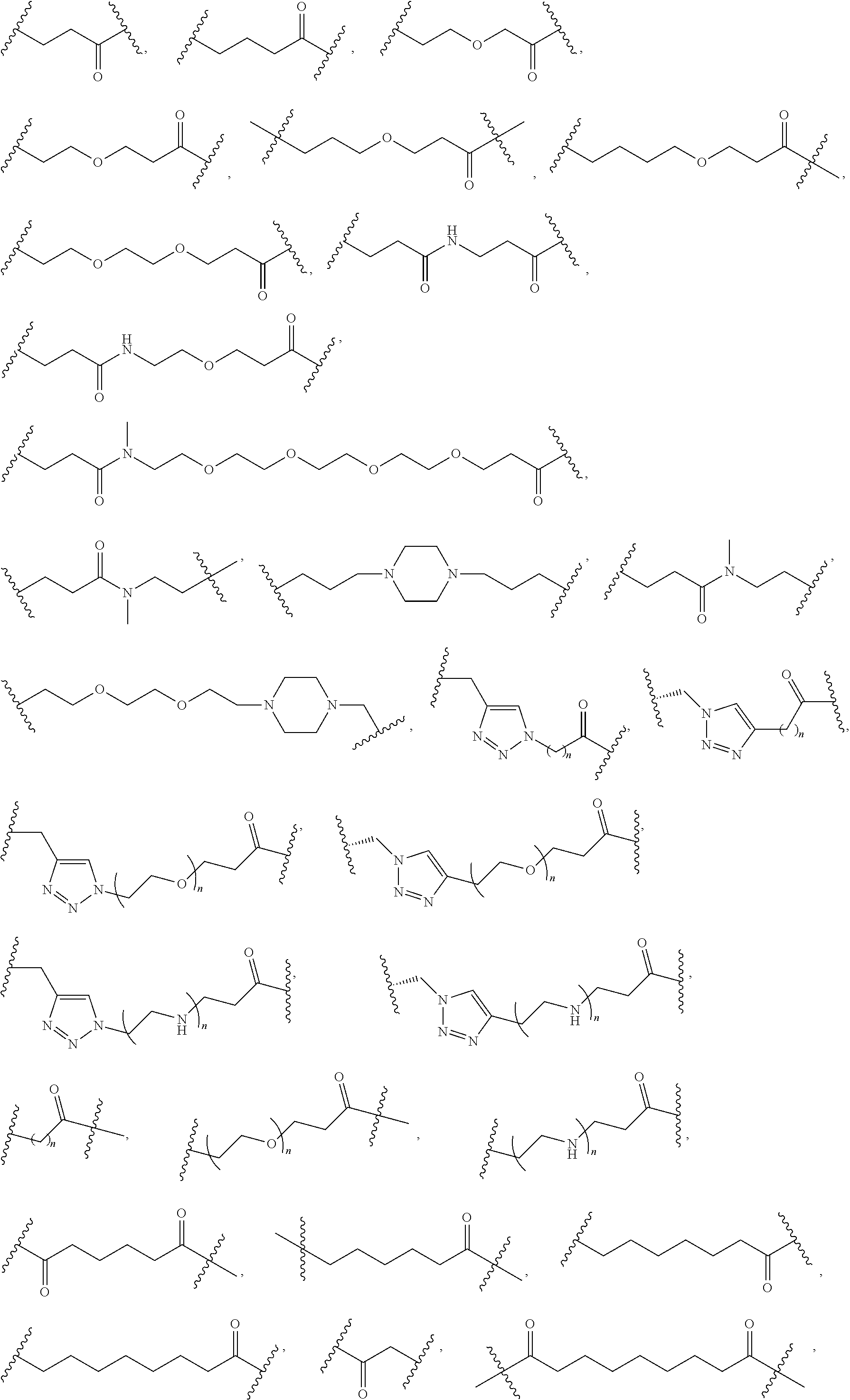

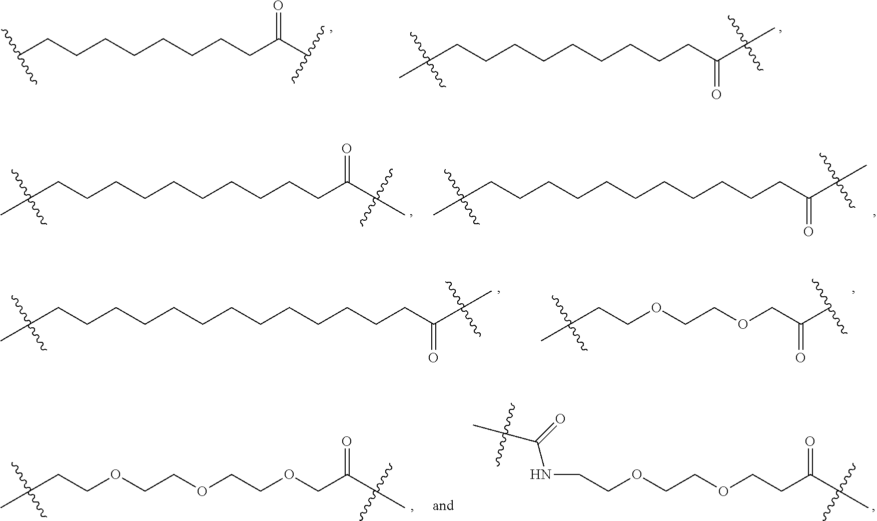

where: n is 0-20 (i.e., n is 0, 1, 2, 3, 4, 5, 6, 7, 8, 9, 10, 11, 12, 13, 14, 15, 16, 17, 18, 19, or 20). In some such embodiments, n is 0-5. In some such embodiments, n is 1 or 2.

In certain embodiments of compounds of Formula (I), L¹, L² and L³, joining together, form a structure which is:

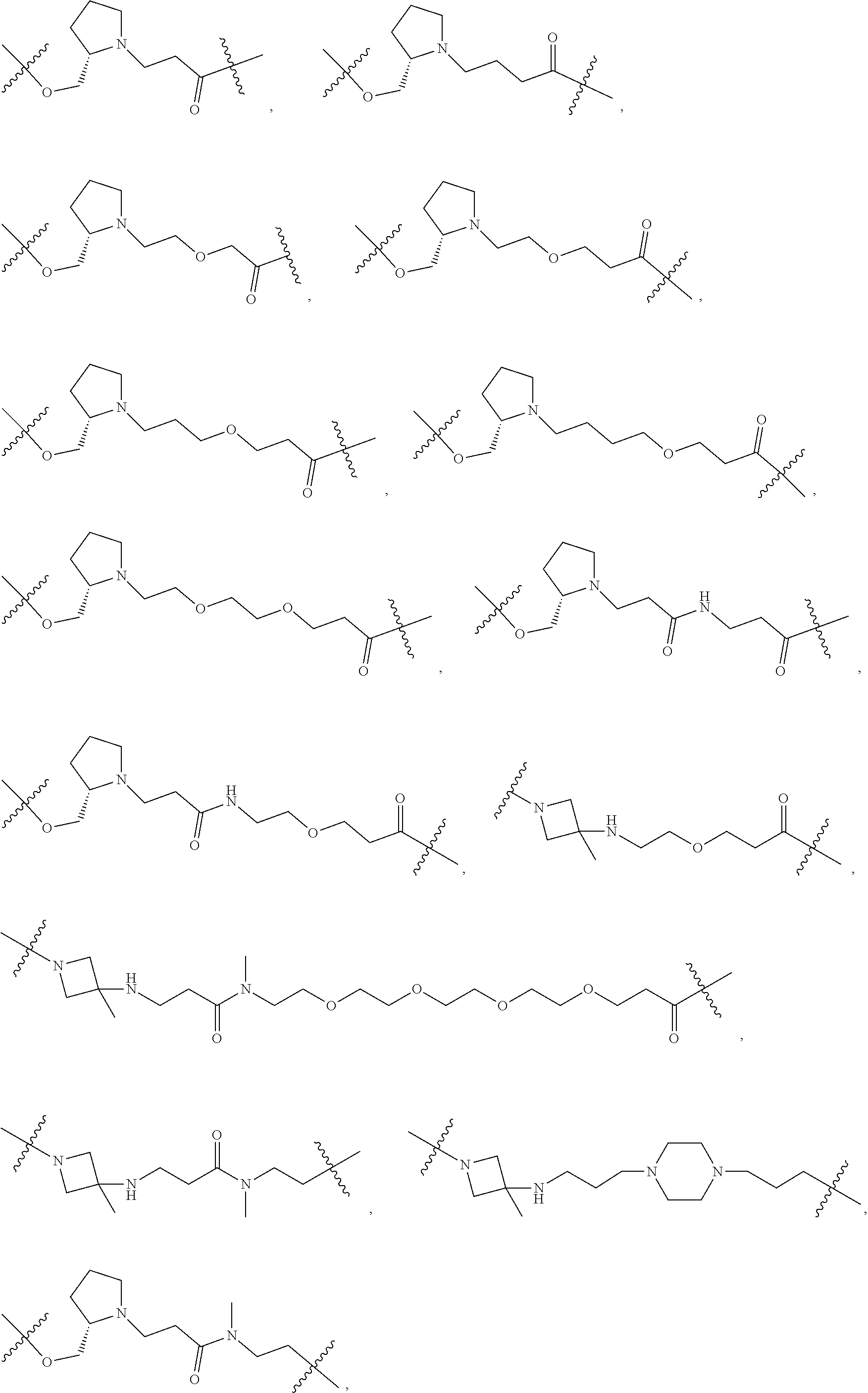

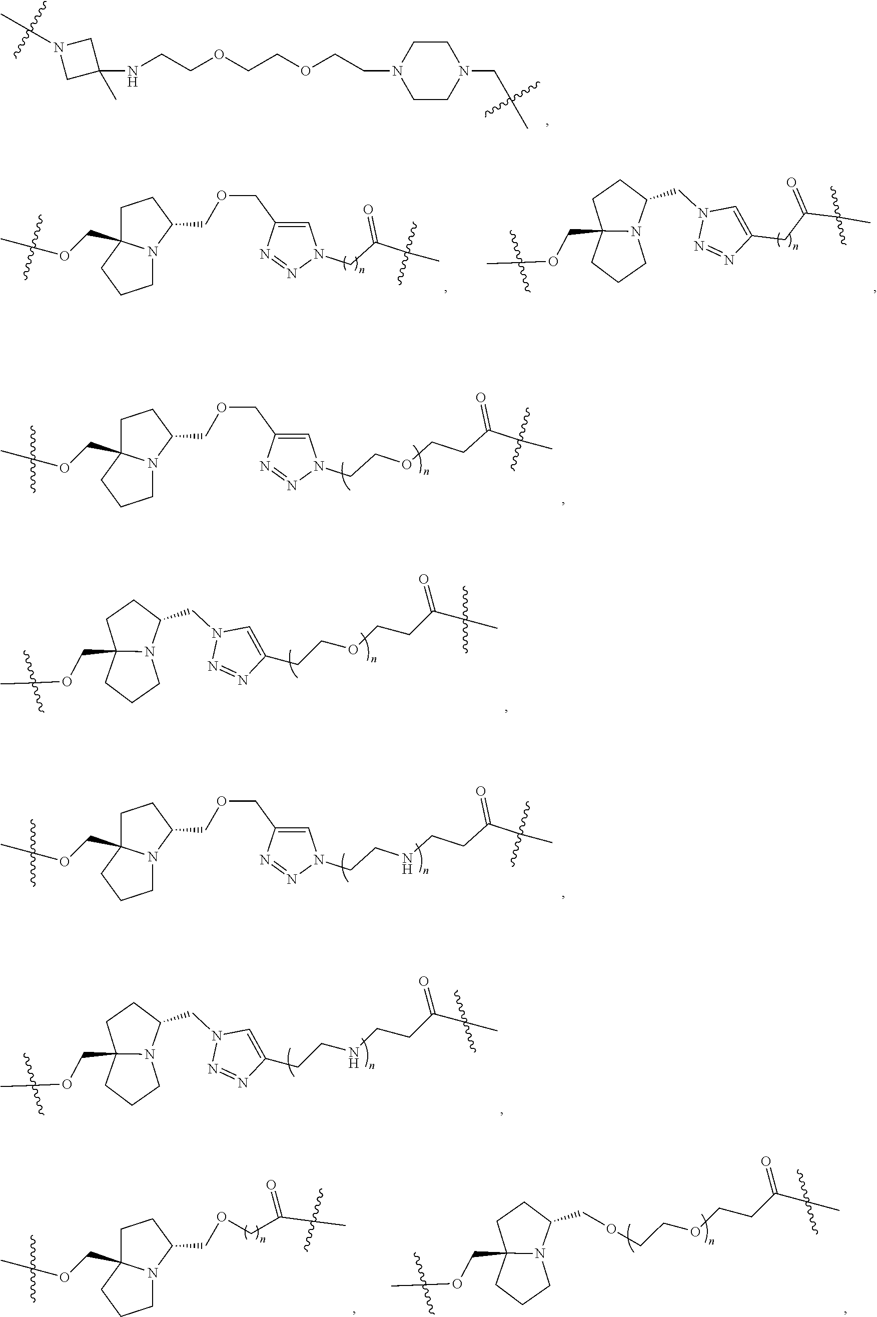

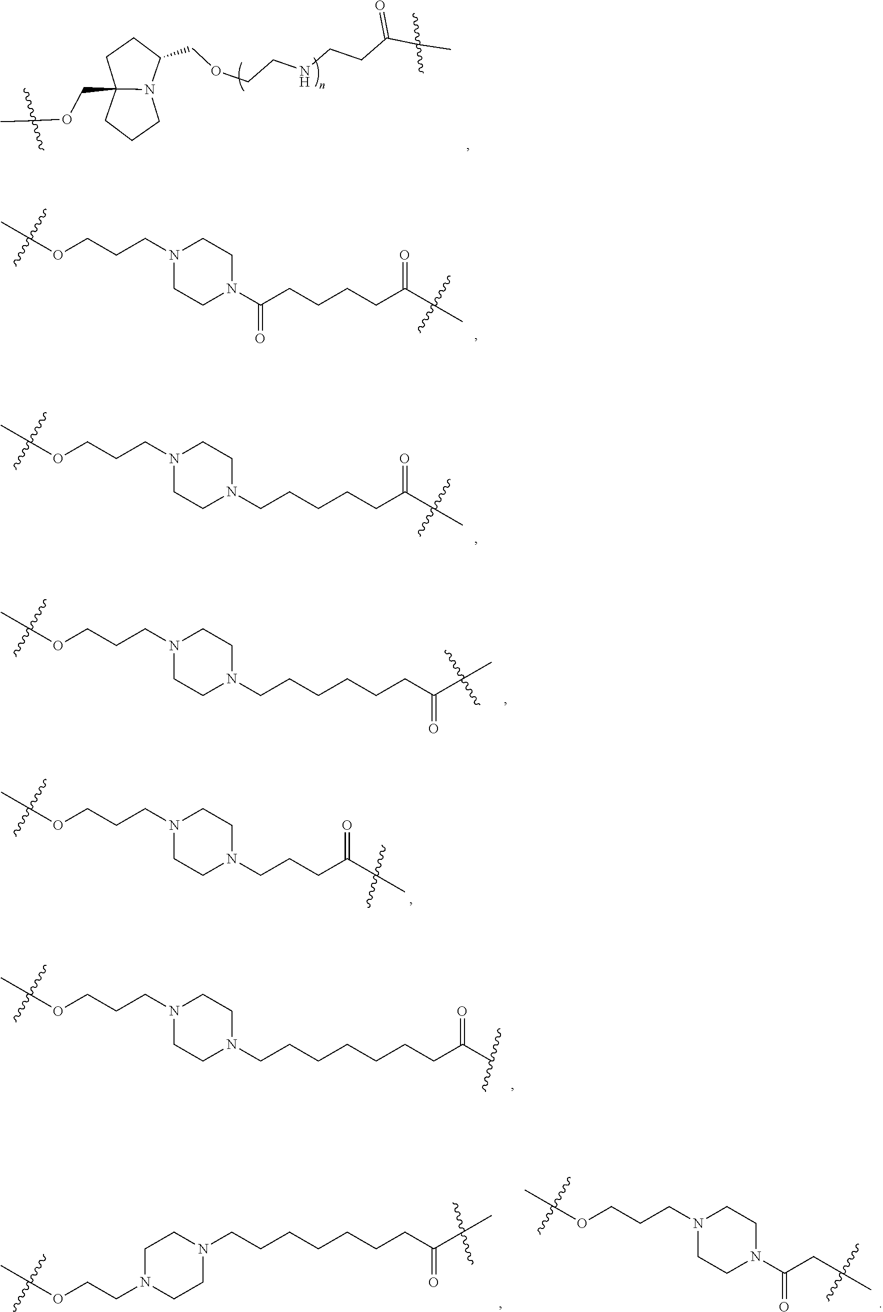

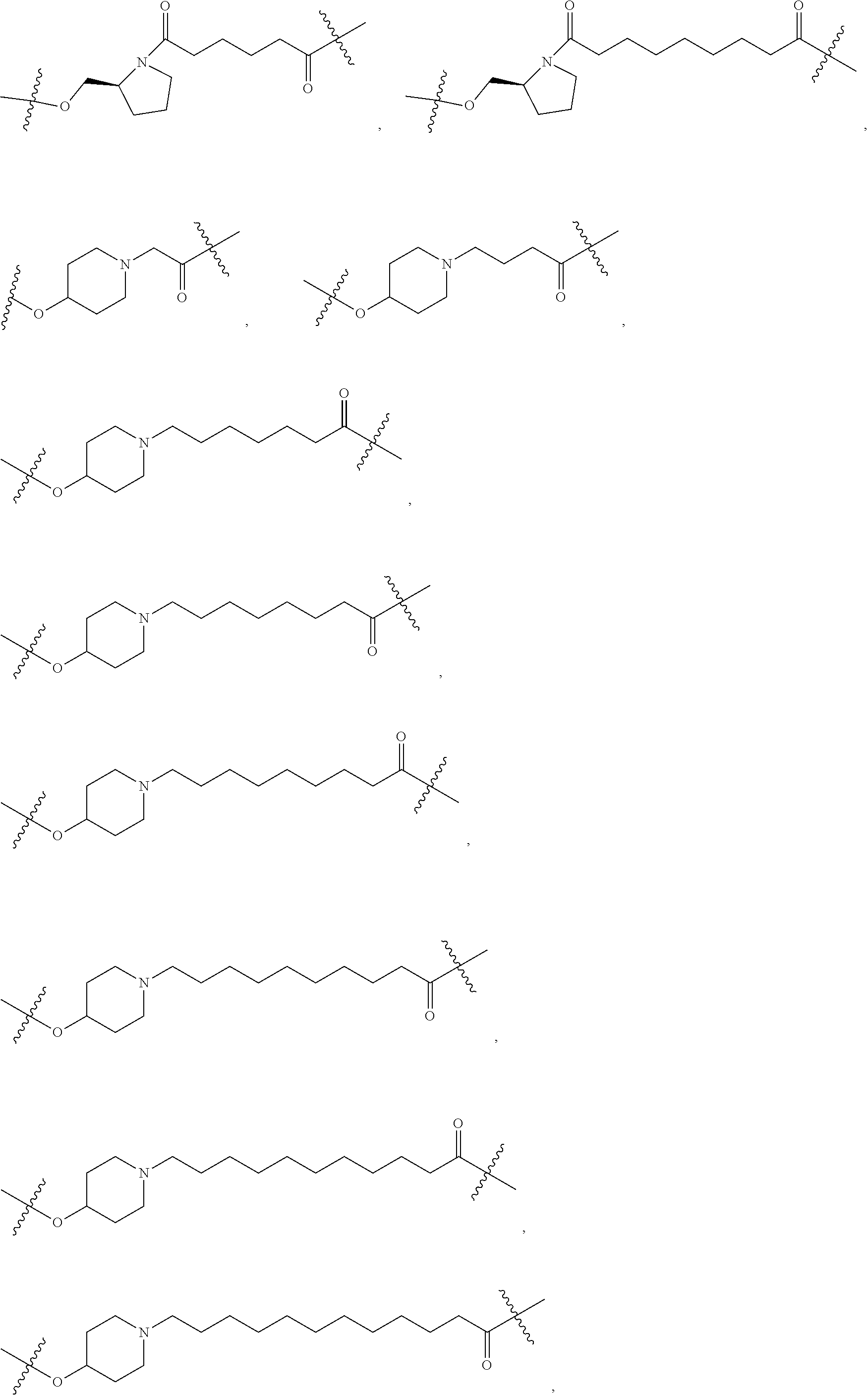

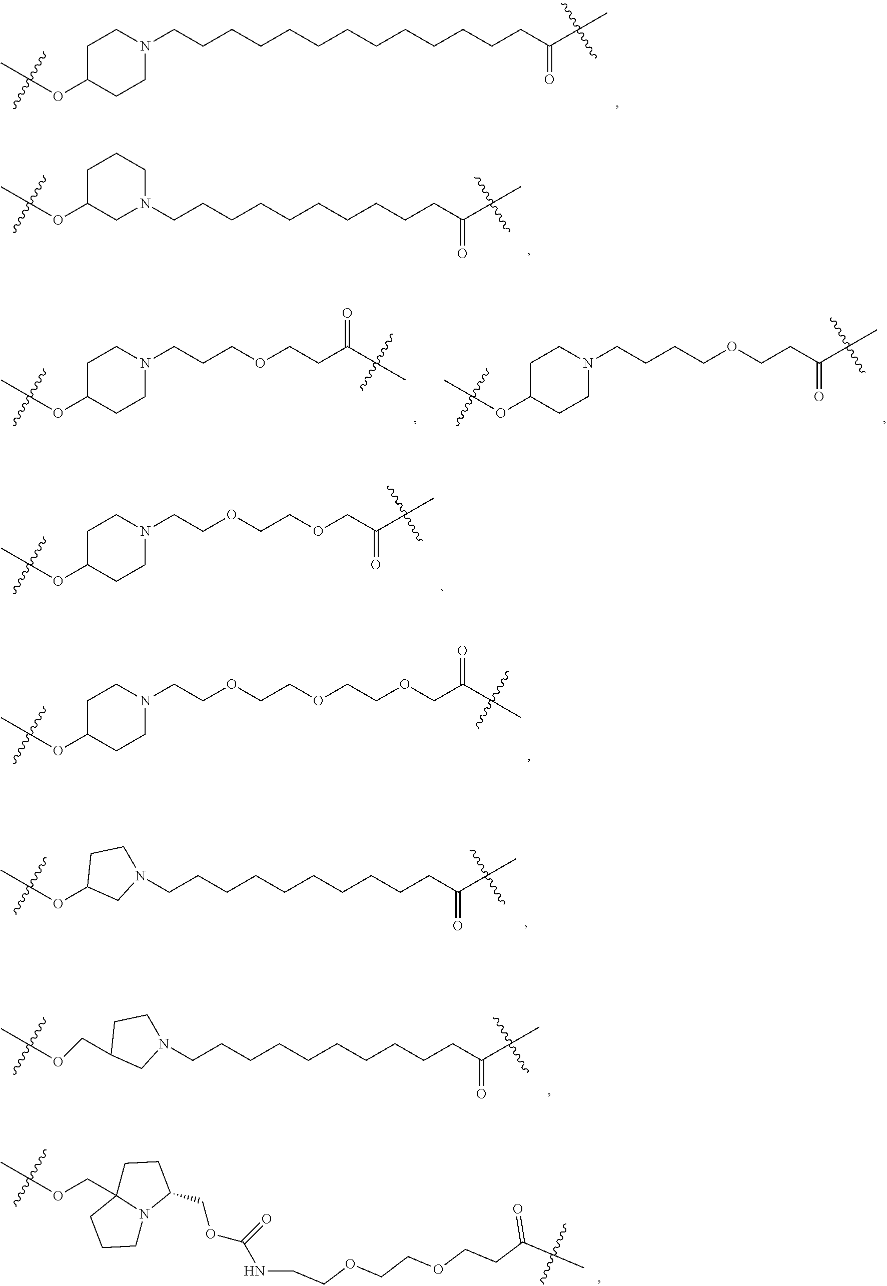

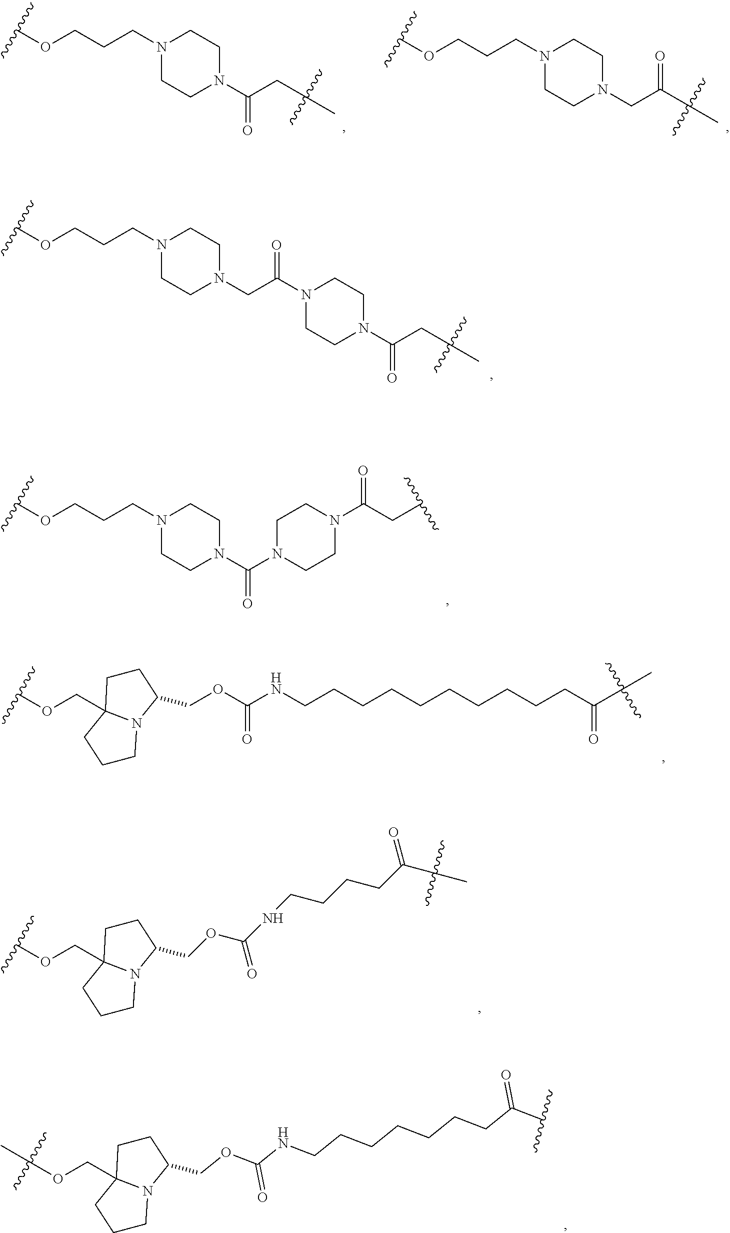

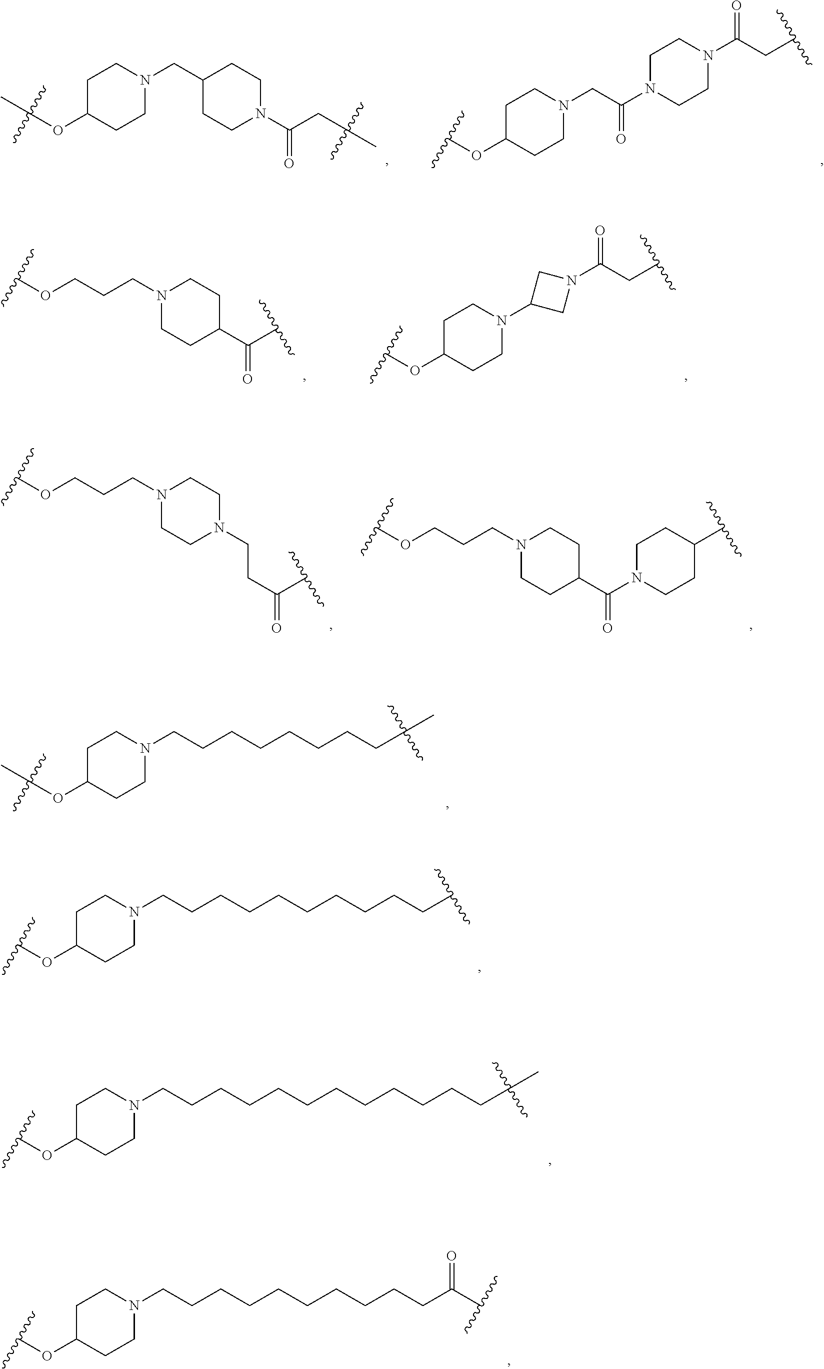

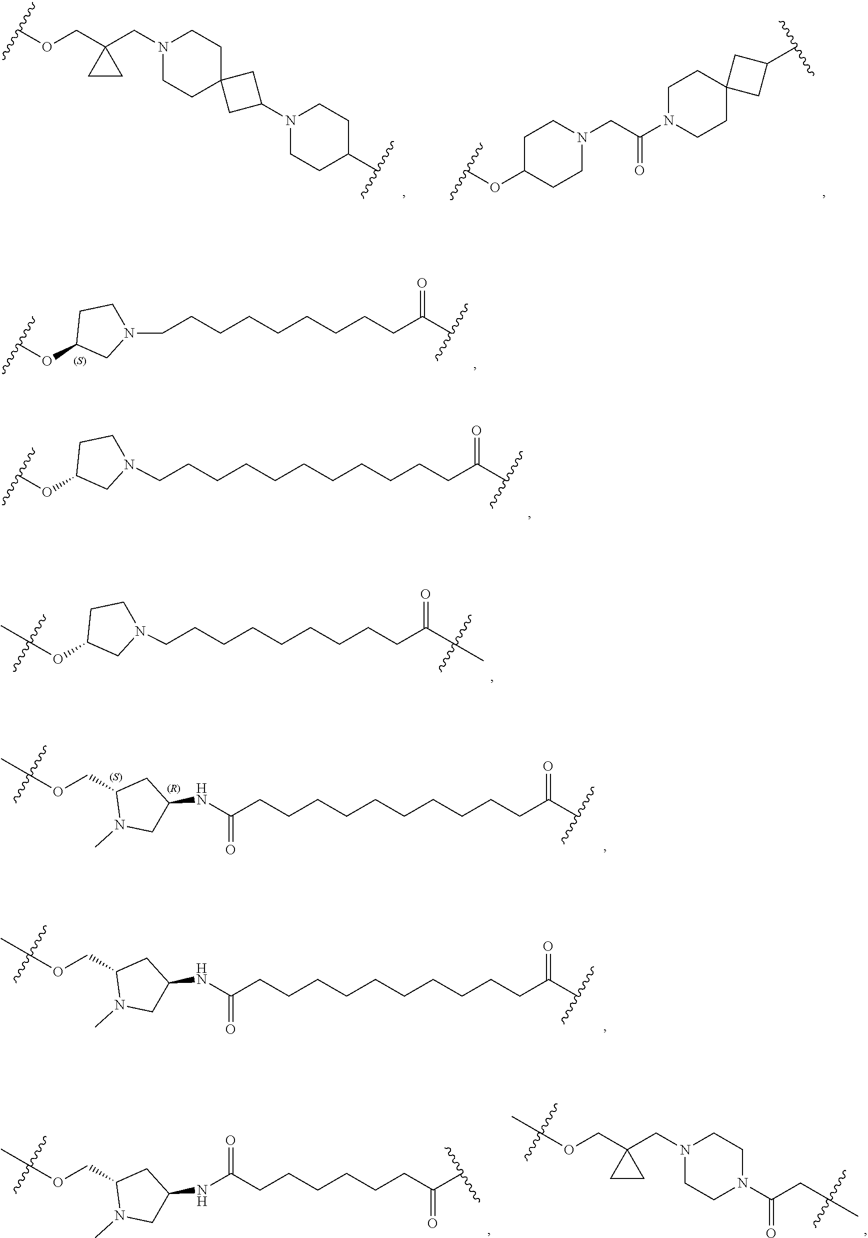

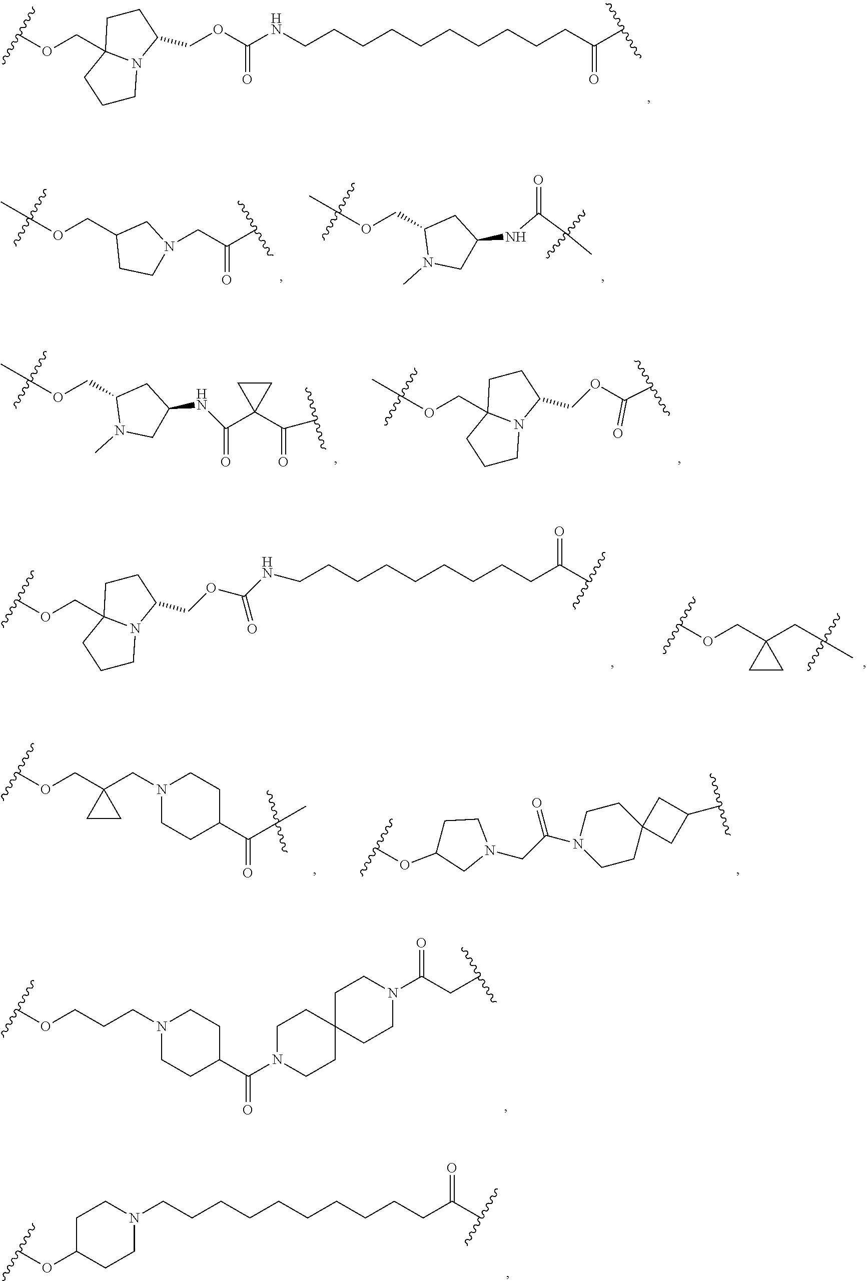

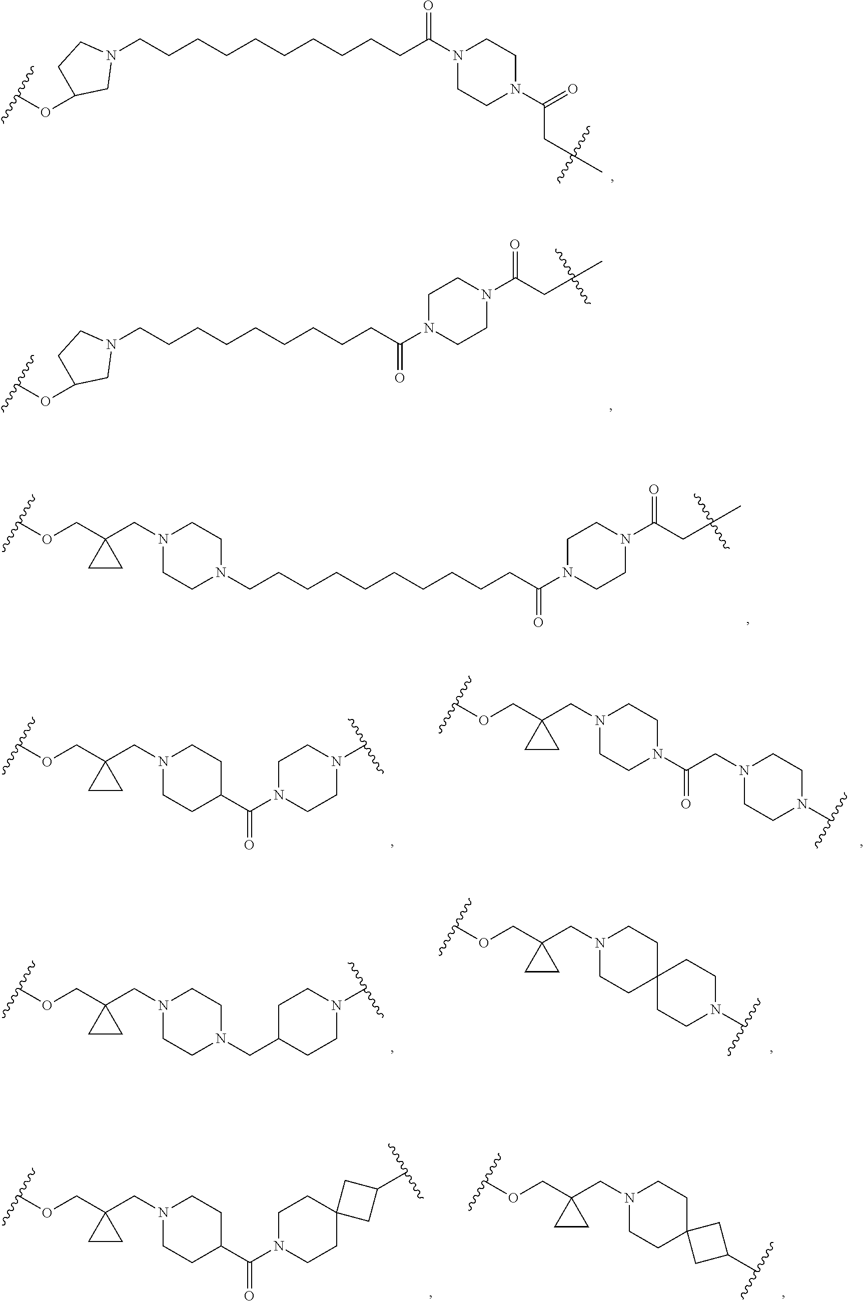

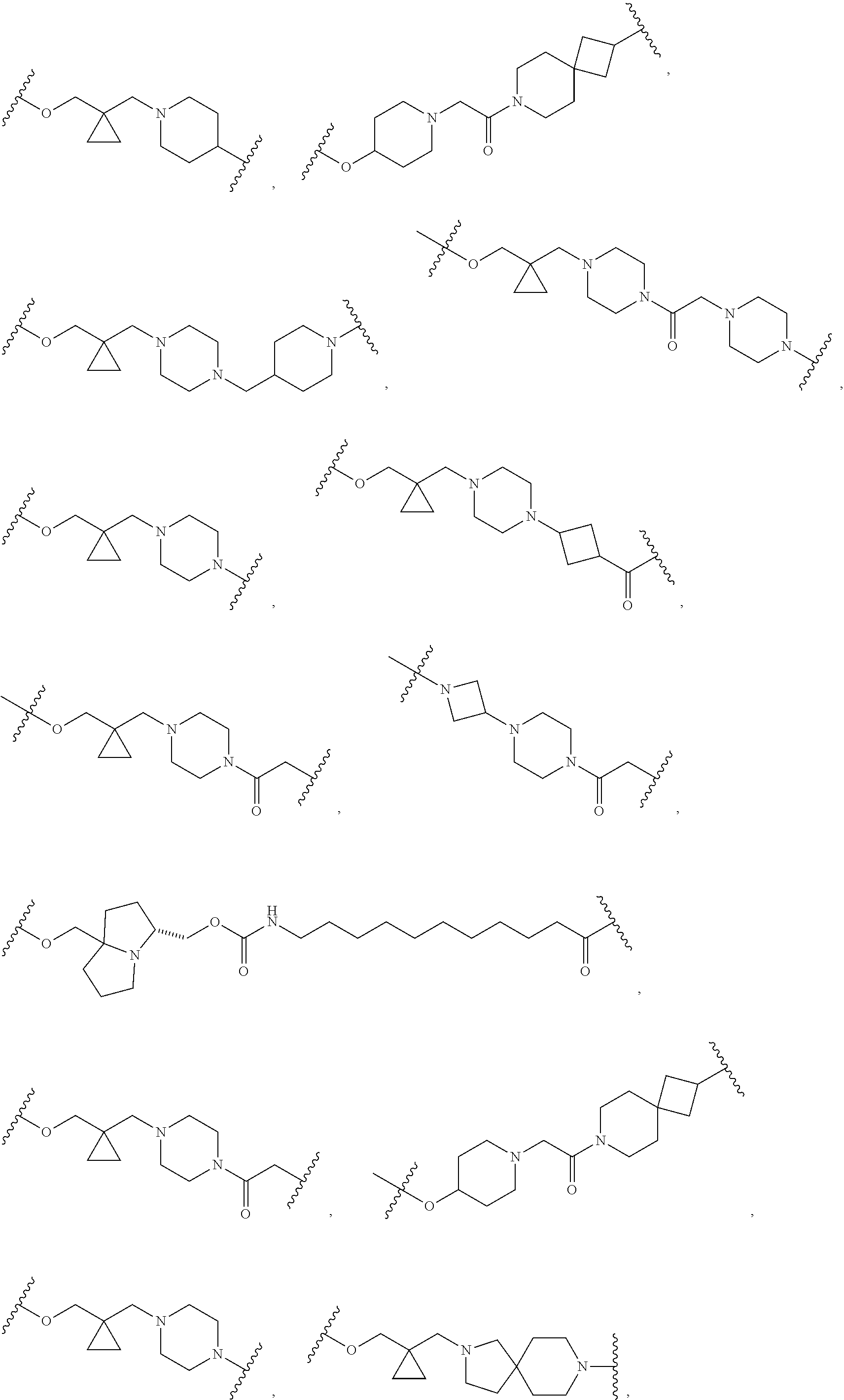

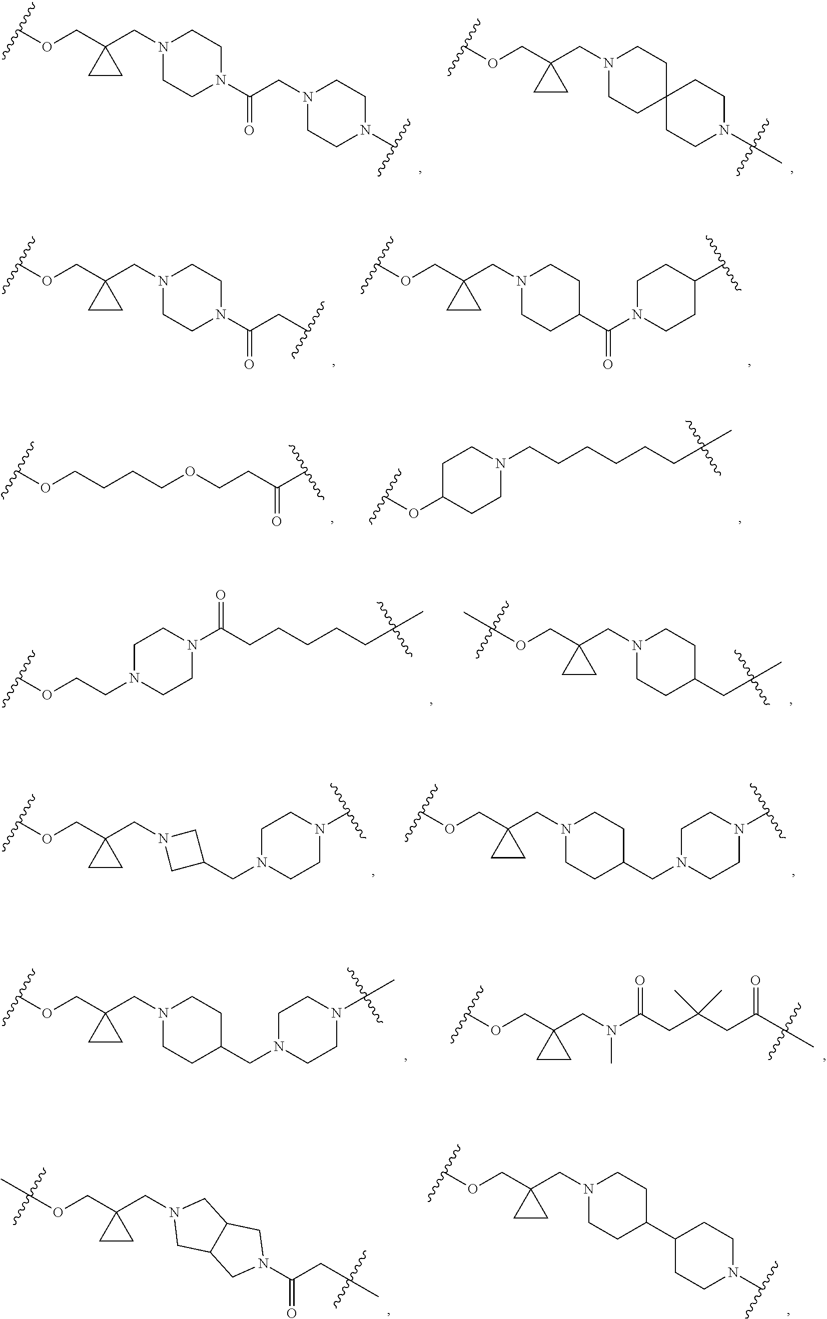

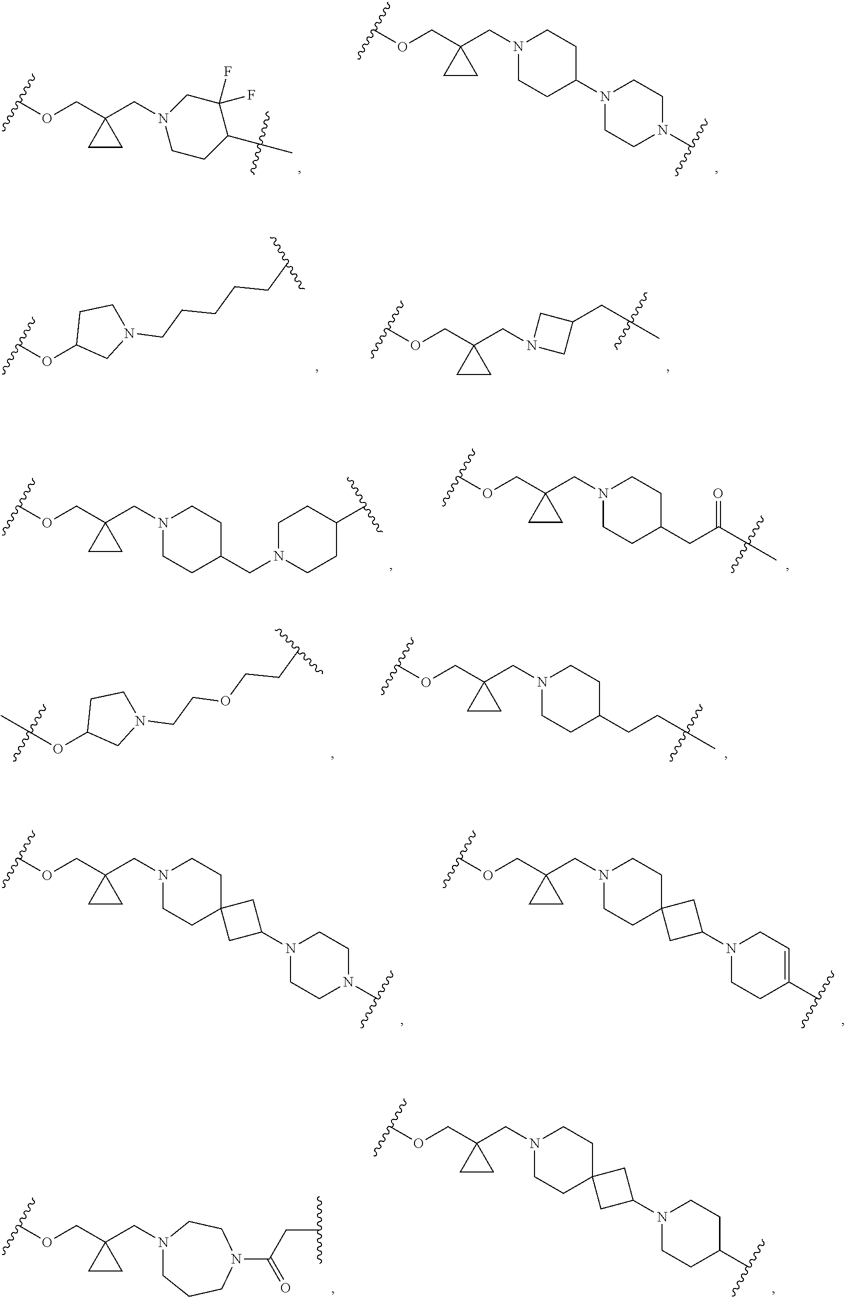

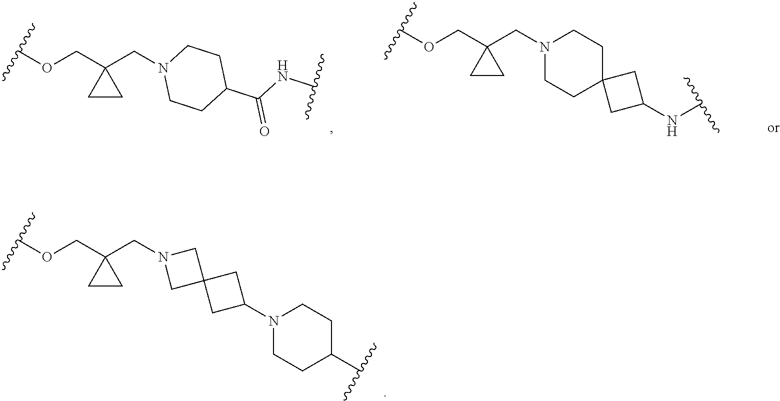 [SMILES: *NC(=O)C1CCN(CC2(CO*)CC2)CC1 ; *NC1CC2(CCN(CC3(CO*)CC3)CC2)C1 ; *OCC1(CN2CC3(CC(N4CCC(*)CC4)C3)C2)CC1]

In certain embodiments of compounds of Formula (I), the bivalent linking group L is:

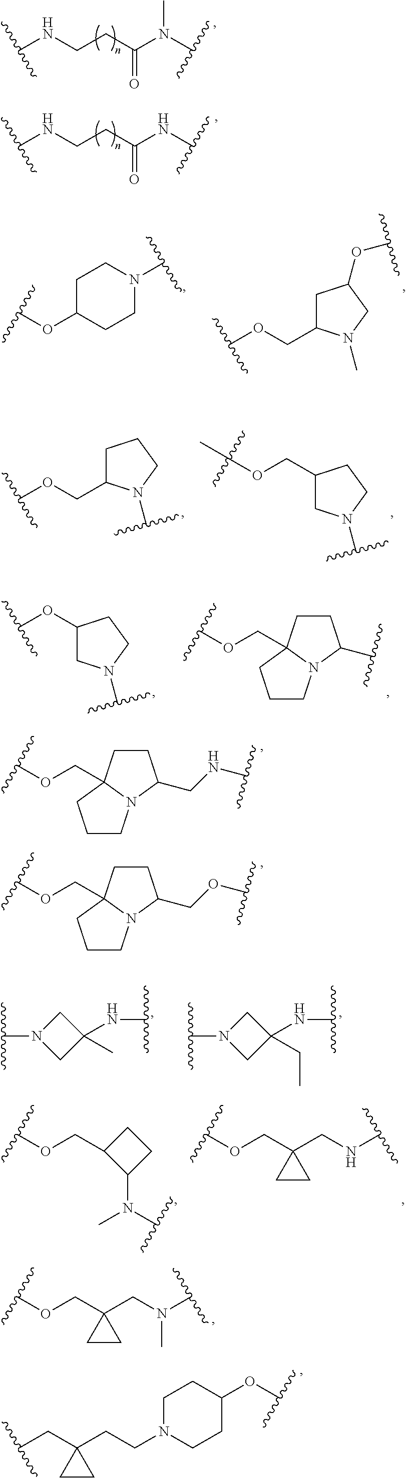

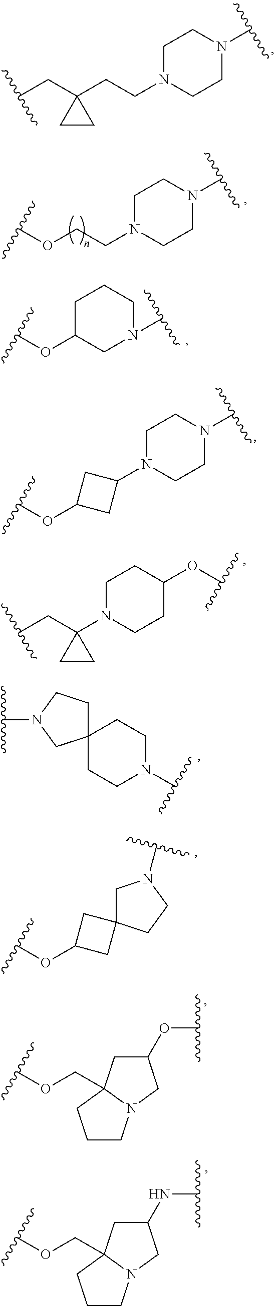

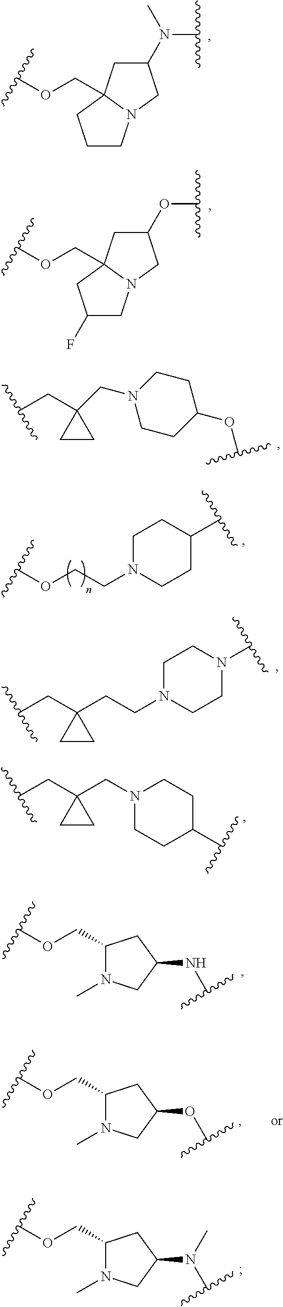

where: n is 0-20 (e.g., 0, 1, 2, 3, 4, 5, 6, 7, 8, 9, 10, 11, 12, 13, 14, 15, 16, 17, 18, 19, or 20). In some such embodiments, n is 0-5. In some such embodiments, n is 1 or 2.

In certain embodiments of compounds of Formula (I), the compound is a compound shown in Table 1, Table 2 or Table 3, or a pharmaceutically acceptable salt, ester, stereoisomer, hydrate, or solvate thereof.

TABLE 1
Structures of exemplary bifunctional compounds in accordance with certain
embodiments of the disclosure.

| Cpd | |
|---|---|
| No. | Structure |
| 1 | 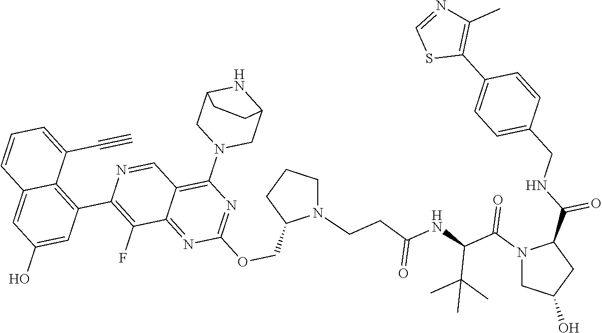 [SMILES: C#Cc1cccc2cc(O)cc(-c3ncc4c(N5CC6CCC(C5)N6)nc(OC[C@@H]5CCCN5CCC(=O)N[C@@H](C(=O)N5C[C@@H](O)C[C@@H]5C(=O)NCc5ccc(-c6scnc6C)cc5)C(C)(C)C)nc4c3F)c12] |
| 2 | 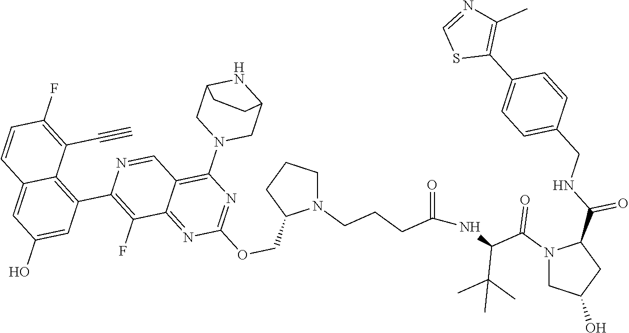 [SMILES: C#Cc1c(F)ccc2cc(O)cc(-c3ncc4c(N5CC6CCC(C5)N6)nc(OC[C@@H]5CCCN5CCCC(=O)N[C@@H](C(=O)N5C[C@@H](O)C[C@@H]5C(=O)NCc5ccc(-c6scnc6C)cc5)C(C)(C)C)nc4c3F)c12] |
| 3 | 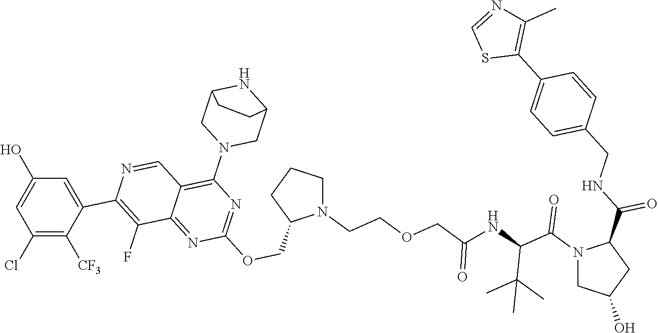 [SMILES: Cc1ncsc1-c1ccc(CNC(=O)[C@H]2C[C@H](O)CN2C(=O)[C@H](NC(=O)COCCN2CCC[C@H]2COc2nc(N3CC4CCC(C3)N4)c3cnc(-c4cc(O)cc(Cl)c4C(F)(F)F)c(F)c3n2)C(C)(C)C)cc1] |
| 4 | 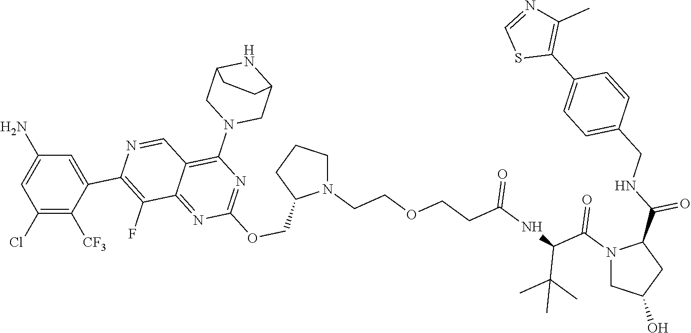 [SMILES: Cc1ncsc1-c1ccc(CNC(=O)[C@H]2C[C@H](O)CN2C(=O)[C@H](NC(=O)CCOCCN2CCC[C@H]2COc2nc(N3CC4CCC(C3)N4)c3cnc(-c4cc(N)cc(Cl)c4C(F)(F)F)c(F)c3n2)C(C)(C)C)cc1] |
| 5 | 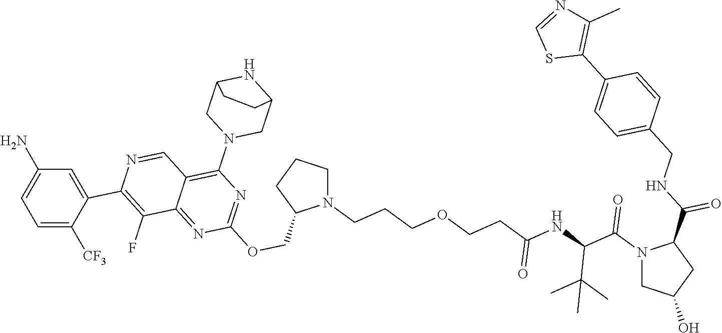 [SMILES: Cc1ncsc1-c1ccc(CNC(=O)[C@H]2C[C@H](O)CN2C(=O)[C@H](NC(=O)CCOCCCN2CCC[C@H]2COc2nc(N3CC4CCC(C3)N4)c3cnc(-c4cc(N)ccc4C(F)(F)F)c(F)c3n2)C(C)(C)C)cc1] |
| 6 | 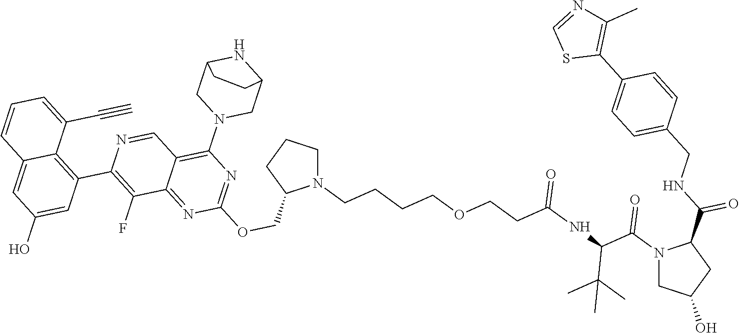 [SMILES: C#Cc1cccc2cc(O)cc(-c3ncc4c(N5CC6CCC(C5)N6)nc(OC[C@@H]5CCCN5CCCCOCCC(=O)N[C@@H](C(=O)N5C[C@@H](O)C[C@@H]5C(=O)NCc5ccc(-c6scnc6C)cc5)C(C)(C)C)nc4c3F)c12] |
| 7 | 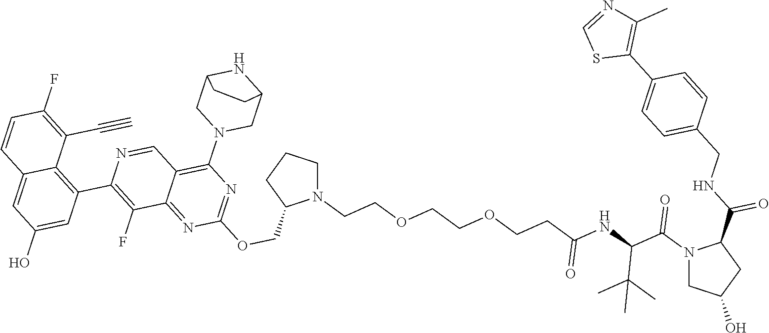 [SMILES: C#Cc1c(F)ccc2cc(O)cc(-c3ncc4c(N5CC6CCC(C5)N6)nc(OC[C@@H]5CCCN5CCOCCOCCC(=O)N[C@@H](C(=O)N5C[C@@H](O)C[C@@H]5C(=O)NCc5ccc(-c6scnc6C)cc5)C(C)(C)C)nc4c3F)c12] |
| 8 | 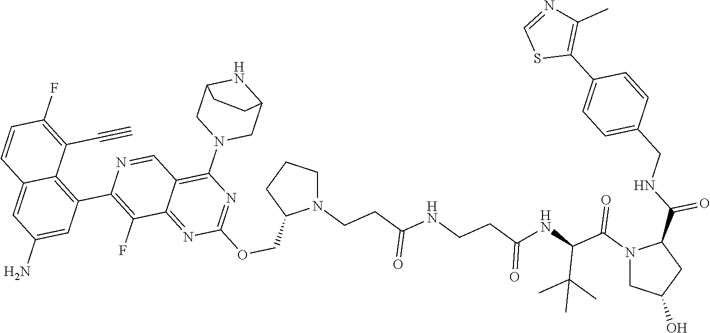 [SMILES: C#Cc1c(F)ccc2cc(N)cc(-c3ncc4c(N5CC6CCC(C5)N6)nc(OC[C@@H]5CCCN5CCC(=O)NCCC(=O)N[C@@H](C(=O)N5C[C@@H](O)C[C@@H]5C(=O)NCc5ccc(-c6scnc6C)cc5)C(C)(C)C)nc4c3F)c12] |
| 9 | 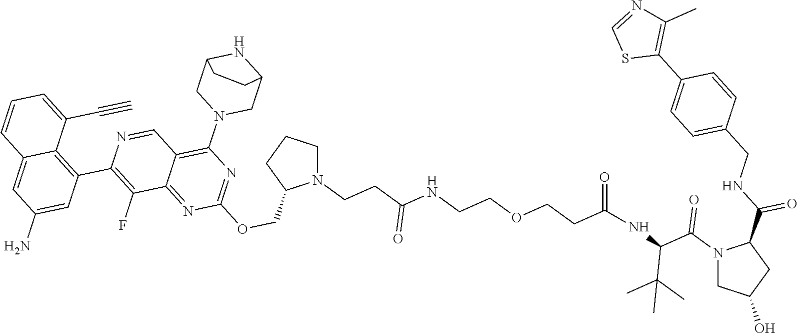 [SMILES: C#Cc1cccc2cc(N)cc(-c3ncc4c(N5CC6CCC(C5)N6)nc(OC[C@@H]5CCCN5CCC(=O)NCCOCCC(=O)N[C@@H](C(=O)N5C[C@@H](O)C[C@@H]5C(=O)NCc5ccc(-c6scnc6C)cc5)C(C)(C)C)nc4c3F)c12] |
| 10 | 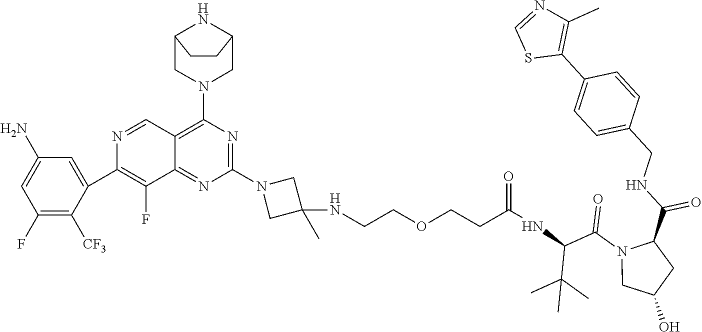 [SMILES: Cc1ncsc1-c1ccc(CNC(=O)[C@H]2C[C@H](O)CN2C(=O)[C@H](NC(=O)CCOCCNC2(C)CN(c3nc(N4CC5CCC(C4)N5)c4cnc(-c5cc(N)cc(F)c5C(F)(F)F)c(F)c4n3)C2)C(C)(C)C)cc1] |
| 11 | 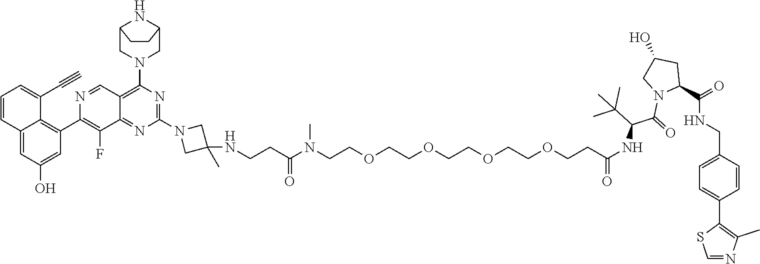 [SMILES: C#Cc1cccc2cc(O)cc(-c3ncc4c(N5CC6CCC(C5)N6)nc(N5CC(C)(NCCC(=O)N(C)CCOCCOCCOCCOCCC(=O)N[C@H](C(=O)N6C[C@H](O)C[C@H]6C(=O)NCc6ccc(-c7scnc7C)cc6)C(C)(C)C)C5)nc4c3F)c12] |
| 12 | 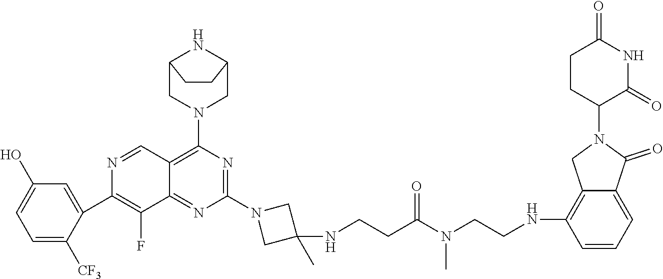 [SMILES: CN(CCNc1cccc2c1CN(C1CCC(=O)NC1=O)C2=O)C(=O)CCNC1(C)CN(c2nc(N3CC4CCC(C3)N4)c3cnc(-c4cc(O)ccc4C(F)(F)F)c(F)c3n2)C1] |
| 13 | 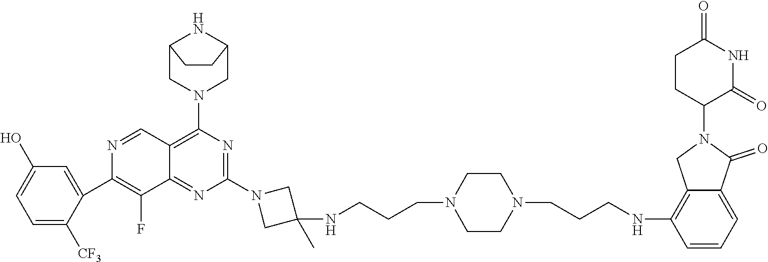 [SMILES: CC1(NCCCN2CCN(CCCNc3cccc4c3CN(C3CCC(=O)NC3=O)C4=O)CC2)CN(c2nc(N3CC4CCC(C3)N4)c3cnc(-c4cc(O)ccc4C(F)(F)F)c(F)c3n2)C1] |
| 14 | 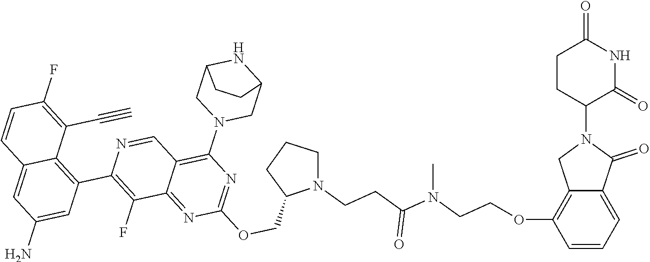 [SMILES: C#Cc1c(F)ccc2cc(N)cc(-c3ncc4c(N5CC6CCC(C5)N6)nc(OC[C@@H]5CCCN5CCC(=O)N(C)CCOc5cccc6c5CN(C5CCC(=O)NC5=O)C6=O)nc4c3F)c12] |
| 15 | 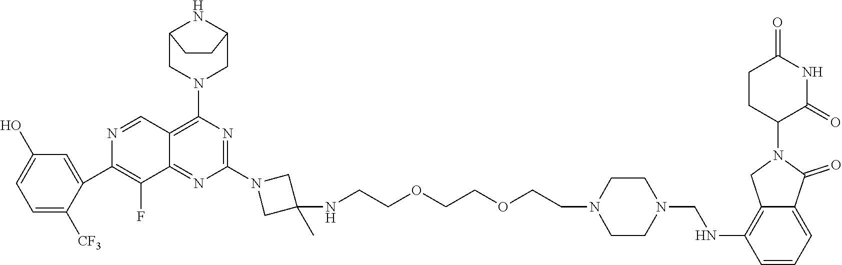 [SMILES: CC1(NCCOCCOCCN2CCN(CNc3cccc4c3CN(C3CCC(=O)NC3=O)C4=O)CC2)CN(c2nc(N3CC4CCC(C3)N4)c3cnc(-c4cc(O)ccc4C(F)(F)F)c(F)c3n2)C1] |
| 16 | 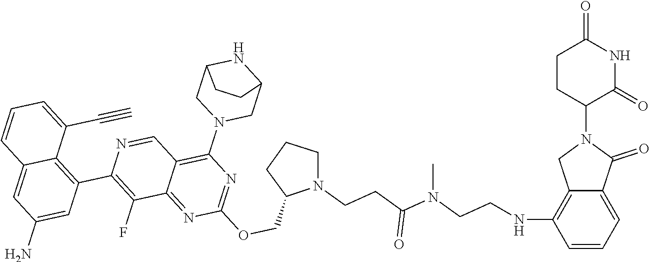 [SMILES: C#Cc1cccc2cc(N)cc(-c3ncc4c(N5CC6CCC(C5)N6)nc(OC[C@@H]5CCCN5CCC(=O)N(C)CCNc5cccc6c5CN(C5CCC(=O)NC5=O)C6=O)nc4c3F)c12] |
| 17 | 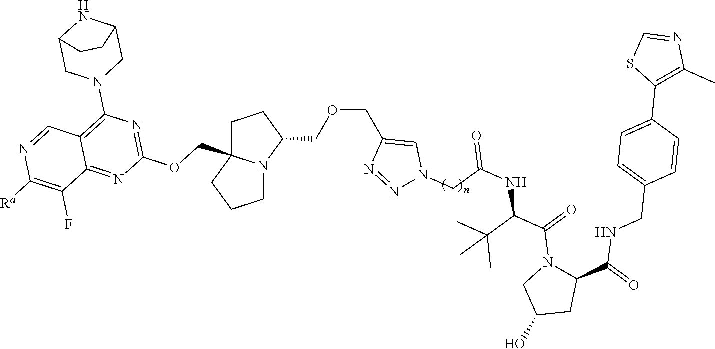 |
| | n = 1-20 R^{a} = |
| | 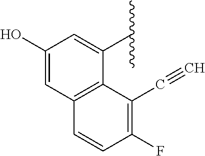 [SMILES: *c1cc(O)cc2ccc(F)c(C#C)c12] |
| | 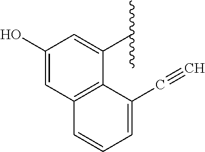 [SMILES: *c1cc(O)cc2cccc(C#C)c12] |
| | 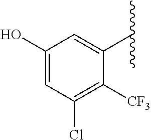 [SMILES: *c1cc(O)cc(Cl)c1C(F)(F)F] |
| | 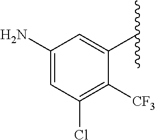 [SMILES: *c1cc(N)cc(Cl)c1C(F)(F)F] |
| | 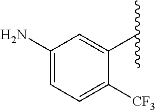 [SMILES: *c1cc(N)ccc1C(F)(F)F] |
| | 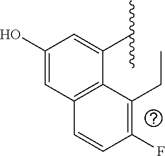 [SMILES: *c1cc(O)cc2ccc(F)c(CC)c12] |
| | 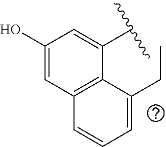 [SMILES: *c1cc(O)cc2cccc(CC)c12] |
| 18 | 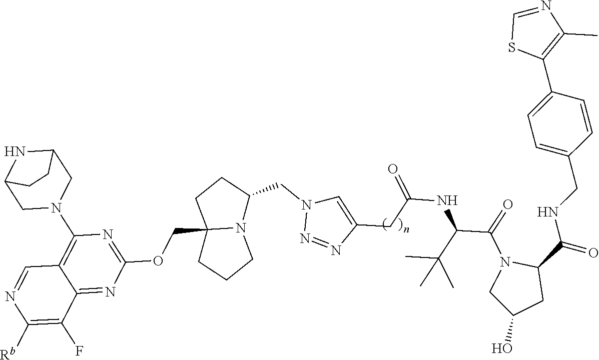 |
| | n = 1-20 R^{b} = |
| | 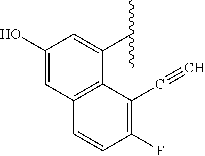 [SMILES: *c1cc(O)cc2ccc(F)c(C#C)c12] |
| | 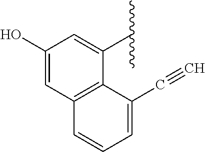 [SMILES: *c1cc(O)cc2cccc(C#C)c12] |
| | 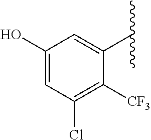 [SMILES: *c1cc(O)cc(Cl)c1C(F)(F)F] |
| | 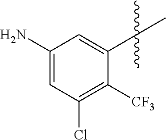 [SMILES: *c1cc(N)cc(Cl)c1C(F)(F)F] |
| | 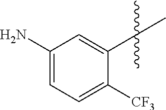 [SMILES: *c1cc(N)ccc1C(F)(F)F] |
| | 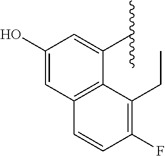 [SMILES: *c1cc(O)cc2ccc(F)c(CC)c12] |
| | 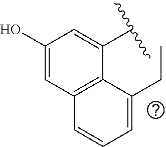 [SMILES: *c1cc(O)cc2cccc(CC)c12] |
| 19 | 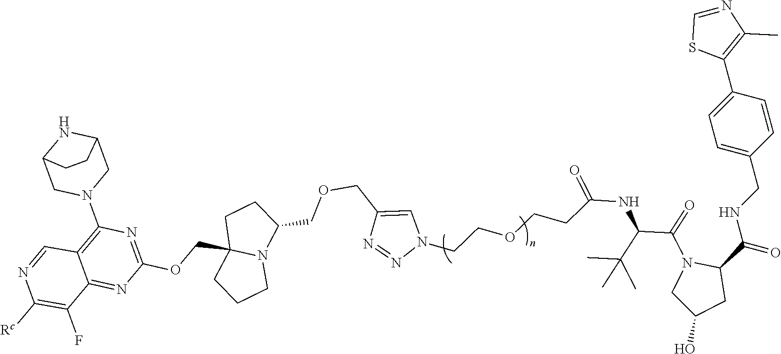 |
| | n = 0-5 R^{c} = |
| | 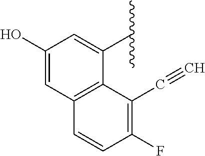 [SMILES: *c1cc(O)cc2ccc(F)c(C#C)c12] |
| | 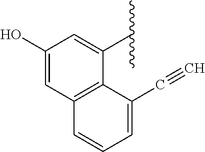 [SMILES: *c1cc(O)cc2cccc(C#C)c12] |
| | 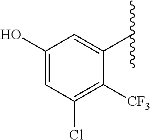 [SMILES: *c1cc(O)cc(Cl)c1C(F)(F)F] |
| | 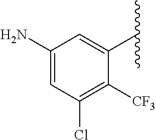 [SMILES: *c1cc(N)cc(Cl)c1C(F)(F)F] |
| | 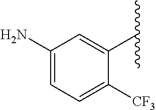 [SMILES: *c1cc(N)ccc1C(F)(F)F] |
| | 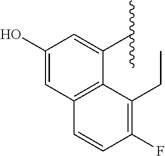 [SMILES: *c1cc(O)cc2ccc(F)c(CC)c12] |
| | 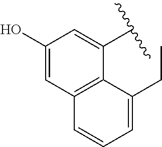 [SMILES: *c1cc(O)cc2cccc(CC)c12] |
| 20 | 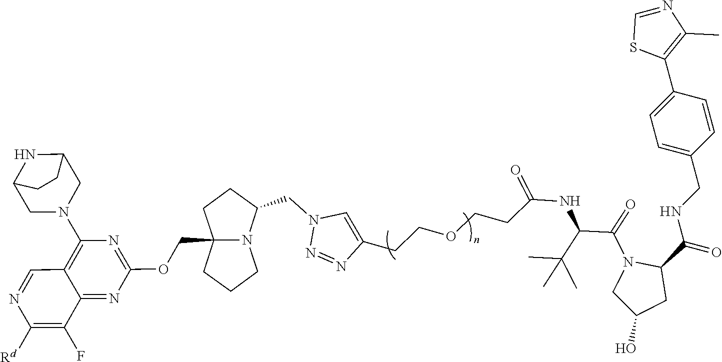 |
| | n = 0-5 R^{d} = |
| | 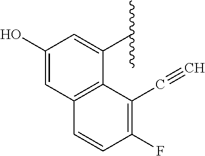 [SMILES: *c1cc(O)cc2ccc(F)c(C#C)c12] |
| | 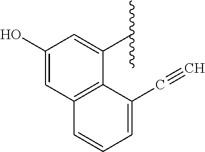 [SMILES: *c1cc(O)cc2cccc(C#C)c12] |
| | 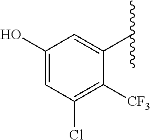 [SMILES: *c1cc(O)cc(Cl)c1C(F)(F)F] |
| | 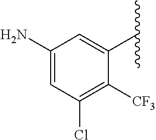 [SMILES: *c1cc(N)cc(Cl)c1C(F)(F)F] |
| | 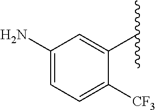 [SMILES: *c1cc(N)ccc1C(F)(F)F] |
| | 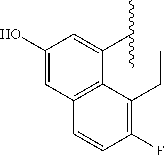 [SMILES: *c1cc(O)cc2ccc(F)c(CC)c12] |
| | 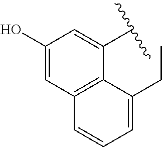 [SMILES: *c1cc(O)cc2cccc(CC)c12] |
| 21 | 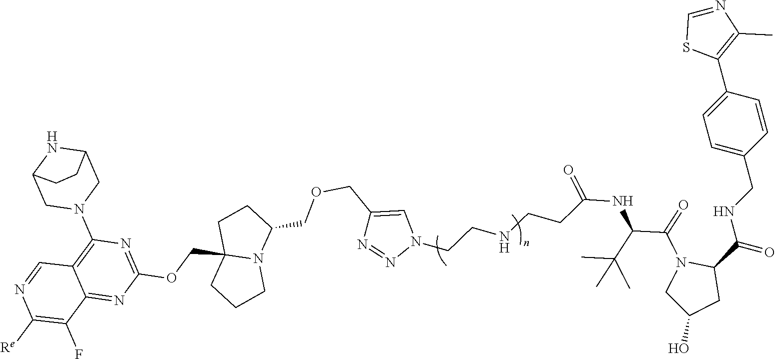 |
| | n = 0-5 R^{e} = |
| | 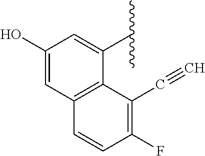 [SMILES: *c1cc(O)cc2ccc(F)c(C#C)c12] |
| | 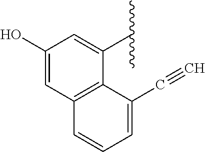 [SMILES: *c1cc(O)cc2cccc(C#C)c12] |
| | 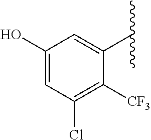 [SMILES: *c1cc(O)cc(Cl)c1C(F)(F)F] |
| | 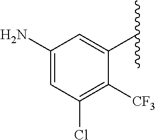 [SMILES: *c1cc(N)cc(Cl)c1C(F)(F)F] |
| | 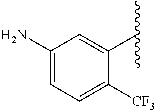 [SMILES: *c1cc(N)ccc1C(F)(F)F] |
| | 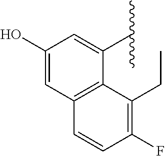 [SMILES: *c1cc(O)cc2ccc(F)c(CC)c12] |
| | 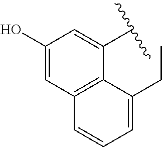 [SMILES: *c1cc(O)cc2cccc(CC)c12] |
| 22 | 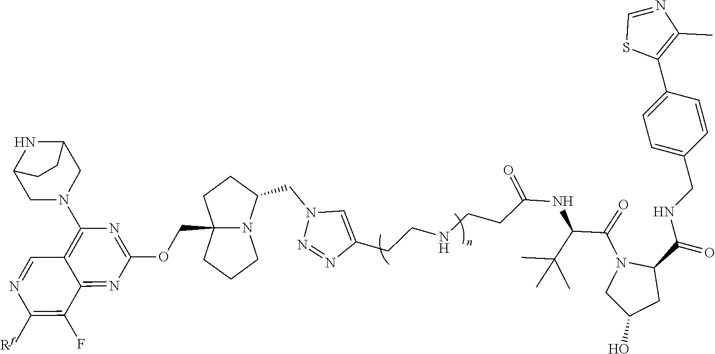 |
| | n = 0-5 R^{f} = |
| | 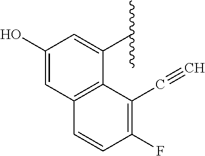 [SMILES: *c1cc(O)cc2ccc(F)c(C#C)c12] |
| | 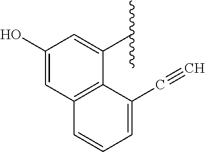 [SMILES: *c1cc(O)cc2cccc(C#C)c12] |
| | 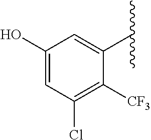 [SMILES: *c1cc(O)cc(Cl)c1C(F)(F)F] |
| | 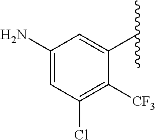 [SMILES: *c1cc(N)cc(Cl)c1C(F)(F)F] |
| | 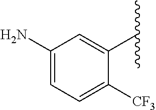 [SMILES: *c1cc(N)ccc1C(F)(F)F] |
| | 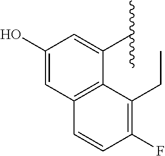 [SMILES: *c1cc(O)cc2ccc(F)c(CC)c12] |
| | 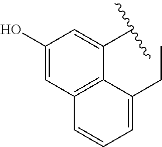 [SMILES: *c1cc(O)cc2cccc(CC)c12] |
| 23 | 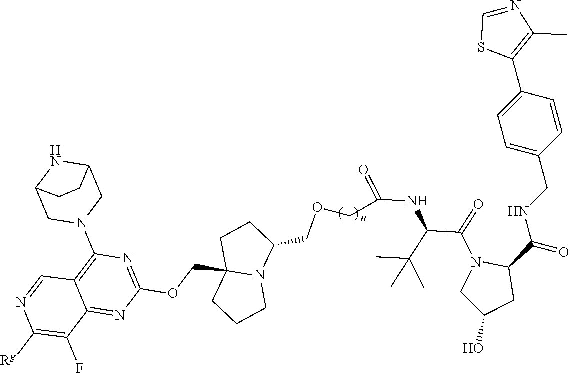 |
| | n = 0-20 R^{g} = |
| | 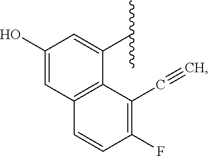 [SMILES: *c1cc(O)cc2ccc(F)c(C#C)c12] |
| | 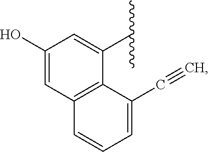 [SMILES: *c1cc(O)cc2cccc(C#C)c12] |
| | 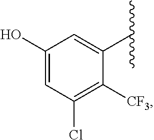 [SMILES: *c1cc(O)cc(Cl)c1C(F)(F)F] |
| | 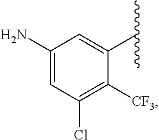 [SMILES: *c1cc(N)cc(Cl)c1C(F)(F)F] |
| | 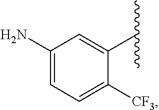 [SMILES: *c1cc(N)ccc1C(F)(F)F] |
| | 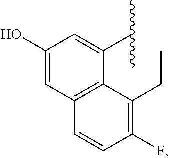 [SMILES: *c1cc(O)cc2ccc(F)c(CC)c12] |
| | 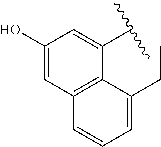 [SMILES: *c1cc(O)cc2cccc(CC)c12] |
| 24 | 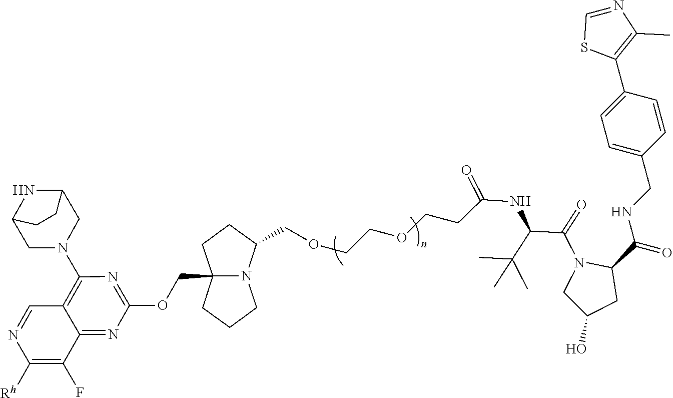 |
| | n = 0-5 R^{h} = |
| | 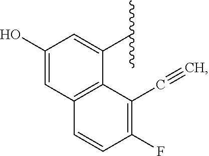 [SMILES: *c1cc(O)cc2ccc(F)c(C#C)c12] |
| | 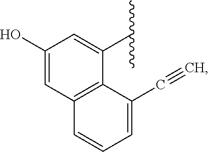 [SMILES: *c1cc(O)cc2cccc(C#C)c12] |
| | 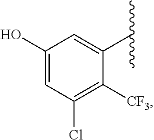 [SMILES: *c1cc(O)cc(Cl)c1C(F)(F)F] |
| | 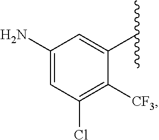 [SMILES: *c1cc(N)cc(Cl)c1C(F)(F)F] |
| | 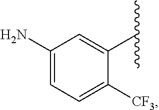 [SMILES: *c1cc(N)ccc1C(F)(F)F] |
| | 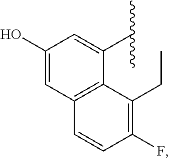 [SMILES: *c1cc(O)cc2ccc(F)c(CC)c12] |
| | 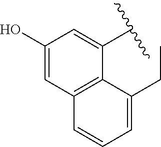 [SMILES: *c1cc(O)cc2cccc(CC)c12] |
| 25 | 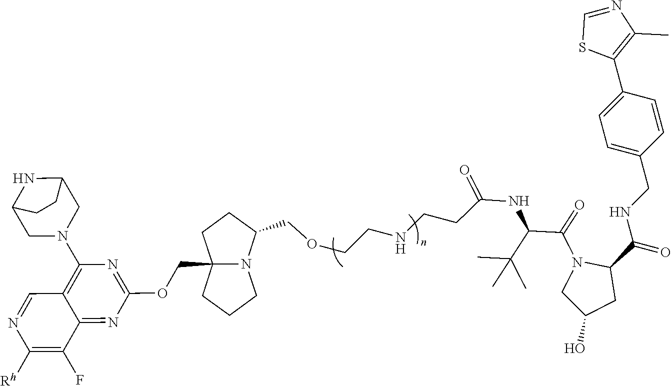 |
| | n = 0-5 R^{h} = |
| | 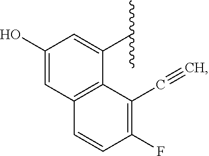 [SMILES: *c1cc(O)cc2ccc(F)c(C#C)c12] |
| | 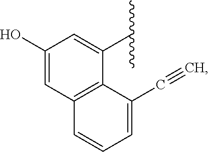 [SMILES: *c1cc(O)cc2cccc(C#C)c12] |
| | 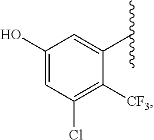 [SMILES: *c1cc(O)cc(Cl)c1C(F)(F)F] |
| | 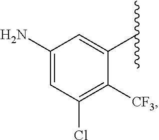 [SMILES: *c1cc(N)cc(Cl)c1C(F)(F)F] |
| | 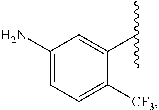 [SMILES: *c1cc(N)ccc1C(F)(F)F] |
| | 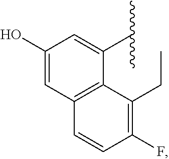 [SMILES: *c1cc(O)cc2ccc(F)c(CC)c12] |
| | 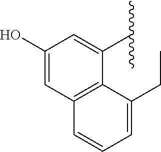 [SMILES: *c1cc(O)cc2cccc(CC)c12] |

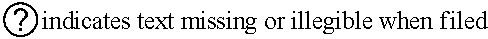

TABLE 2
Structures of exemplary bifunctional compounds in accordance with certain
embodiments of the disclosure.

| Cpd | |
|---|---|
| No. | Structure |
| 26 | 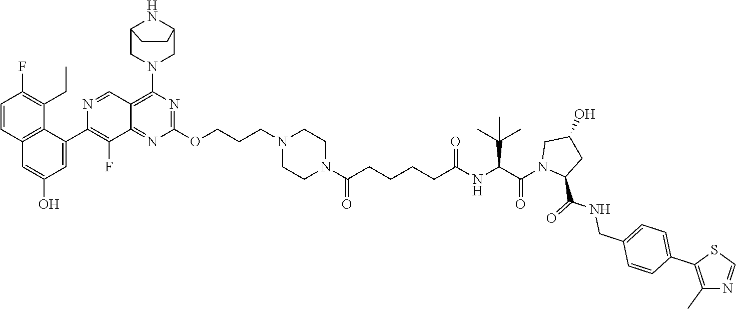 [SMILES: CCc1c(F)ccc2cc(O)cc(-c3ncc4c(N5CC6CCC(C5)N6)nc(OCCCN5CCN(C(=O)CCCCC(=O)N[C@H](C(=O)N6C[C@H](O)C[C@H]6C(=O)NCc6ccc(-c7scnc7C)cc6)C(C)(C)C)CC5)nc4c3F)c12] |
| 27 | 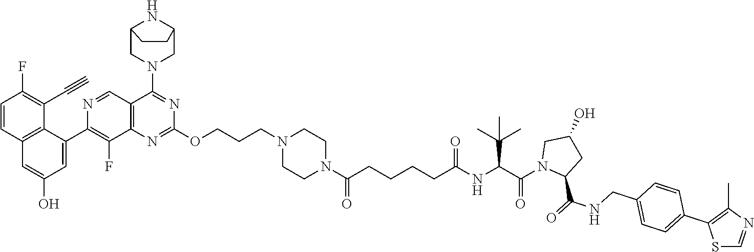 [SMILES: C#Cc1c(F)ccc2cc(O)cc(-c3ncc4c(N5CC6CCC(C5)N6)nc(OCCCN5CCN(C(=O)CCCCC(=O)N[C@H](C(=O)N6C[C@H](O)C[C@H]6C(=O)NCc6ccc(-c7scnc7C)cc6)C(C)(C)C)CC5)nc4c3F)c12] |
| 28 | 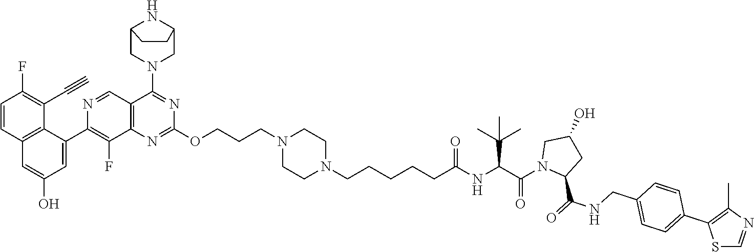 [SMILES: C#Cc1c(F)ccc2cc(O)cc(-c3ncc4c(N5CC6CCC(C5)N6)nc(OCCCN5CCN(CCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 29 | 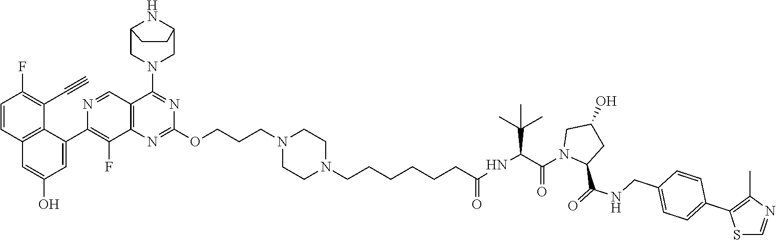 [SMILES: C#Cc1c(F)ccc2cc(O)cc(-c3ncc4c(N5CC6CCC(C5)N6)nc(OCCCN5CCN(CCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 30 | 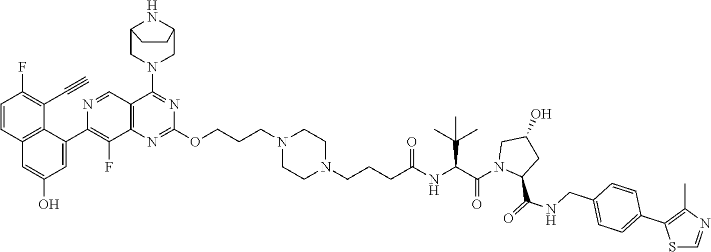 [SMILES: C#Cc1c(F)ccc2cc(O)cc(-c3ncc4c(N5CC6CCC(C5)N6)nc(OCCCN5CCN(CCCC(=O)N[C@H](C(=O)N6C[C@H](O)C[C@H]6C(=O)NCc6ccc(-c7scnc7C)cc6)C(C)(C)C)CC5)nc4c3F)c12] |
| 31 | 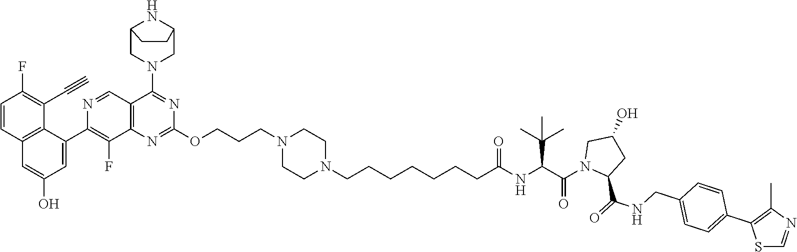 [SMILES: C#Cc1c(F)ccc2cc(O)cc(-c3ncc4c(N5CC6CCC(C5)N6)nc(OCCCN5CCN(CCCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 32 | 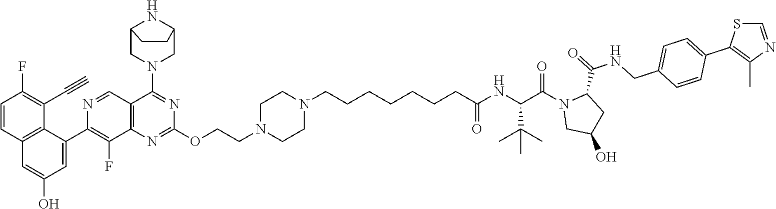 [SMILES: C#Cc1c(F)ccc2cc(O)cc(-c3ncc4c(N5CC6CCC(C5)N6)nc(OCCN5CCN(CCCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 33 | 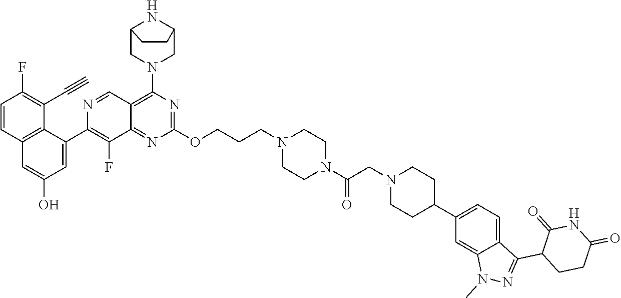 [SMILES: C#Cc1c(F)ccc2cc(O)cc(-c3ncc4c(N5CC6CCC(C5)N6)nc(OCCCN5CCN(C(=O)CN6CCC(c7ccc8c(C9CCC(=O)NC9=O)nn(C)c8c7)CC6)CC5)nc4c3F)c12] |
| 34 | 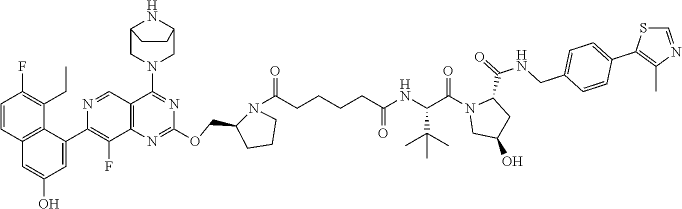 [SMILES: CCc1c(F)ccc2cc(O)cc(-c3ncc4c(N5CC6CCC(C5)N6)nc(OC[C@@H]5CCCN5C(=O)CCCCC(=O)N[C@H](C(=O)N5C[C@H](O)C[C@H]5C(=O)NCc5ccc(-c6scnc6C)cc5)C(C)(C)C)nc4c3F)c12] |
| 35 | 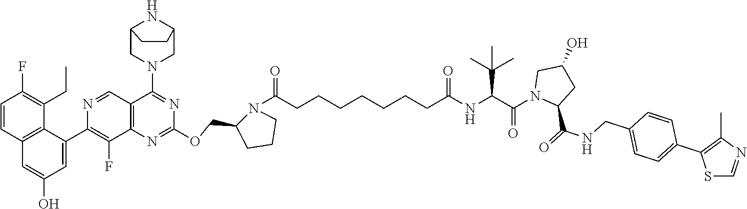 [SMILES: CCc1c(F)ccc2cc(O)cc(-c3ncc4c(N5CC6CCC(C5)N6)nc(OC[C@@H]5CCCN5C(=O)CCCCCCCC(=O)N[C@H](C(=O)N5C[C@H](O)C[C@H]5C(=O)NCc5ccc(-c6scnc6C)cc5)C(C)(C)C)nc4c3F)c12] |
| 36 | 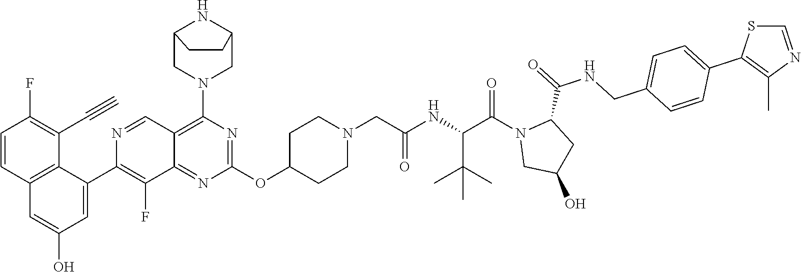 [SMILES: C#Cc1c(F)ccc2cc(O)cc(-c3ncc4c(N5CC6CCC(C5)N6)nc(OC5CCN(CC(=O)N[C@H](C(=O)N6C[C@H](O)C[C@H]6C(=O)NCc6ccc(-c7scnc7C)cc6)C(C)(C)C)CC5)nc4c3F)c12] |
| 37 | 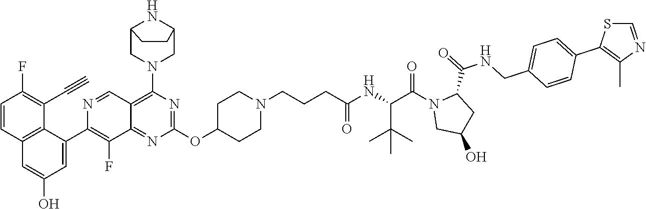 [SMILES: C#Cc1c(F)ccc2cc(O)cc(-c3ncc4c(N5CC6CCC(C5)N6)nc(OC5CCN(CCCC(=O)N[C@H](C(=O)N6C[C@H](O)C[C@H]6C(=O)NCc6ccc(-c7scnc7C)cc6)C(C)(C)C)CC5)nc4c3F)c12] |
| 38 | 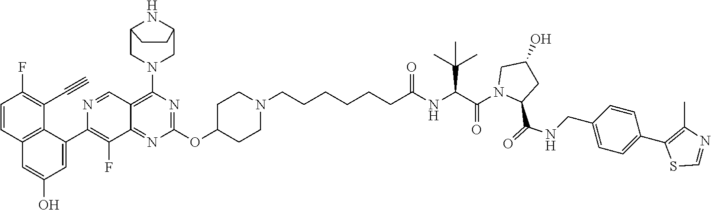 [SMILES: C#Cc1c(F)ccc2cc(O)cc(-c3ncc4c(N5CC6CCC(C5)N6)nc(OC5CCN(CCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 39 | 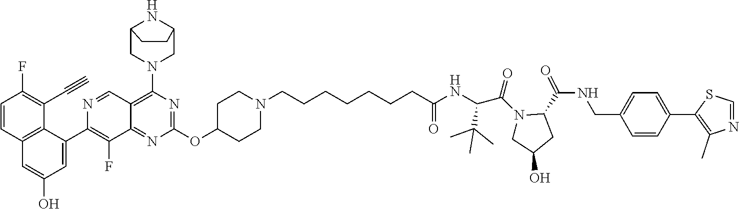 [SMILES: C#Cc1c(F)ccc2cc(O)cc(-c3ncc4c(N5CC6CCC(C5)N6)nc(OC5CCN(CCCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 40 | 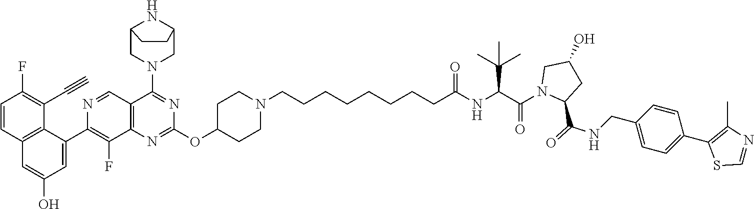 [SMILES: C#Cc1c(F)ccc2cc(O)cc(-c3ncc4c(N5CC6CCC(C5)N6)nc(OC5CCN(CCCCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 41 | 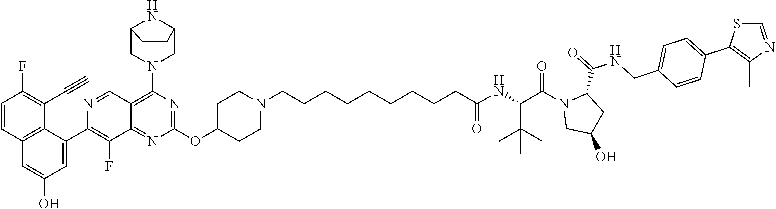 [SMILES: C#Cc1c(F)ccc2cc(O)cc(-c3ncc4c(N5CC6CCC(C5)N6)nc(OC5CCN(CCCCCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 42 | 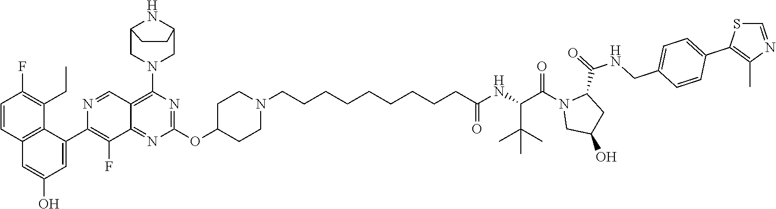 [SMILES: CCc1c(F)ccc2cc(O)cc(-c3ncc4c(N5CC6CCC(C5)N6)nc(OC5CCN(CCCCCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 43 | 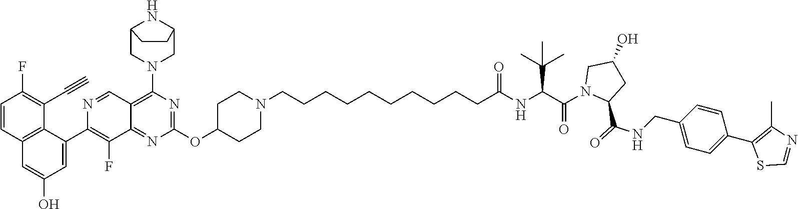 [SMILES: C#Cc1c(F)ccc2cc(O)cc(-c3ncc4c(N5CC6CCC(C5)N6)nc(OC5CCN(CCCCCCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 44 | 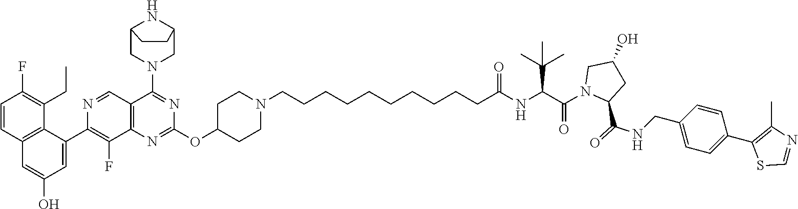 [SMILES: CCc1c(F)ccc2cc(O)cc(-c3ncc4c(N5CC6CCC(C5)N6)nc(OC5CCN(CCCCCCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 45 | 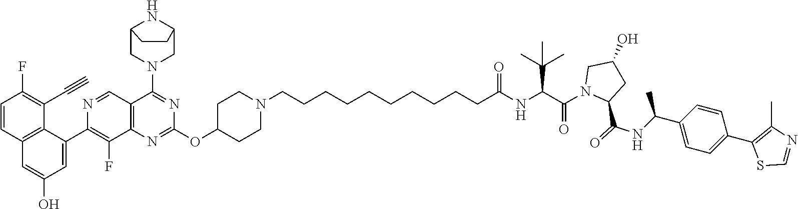 [SMILES: C#Cc1c(F)ccc2cc(O)cc(-c3ncc4c(N5CC6CCC(C5)N6)nc(OC5CCN(CCCCCCCCCCC(=O)N[C@H](C(=O)N6C[C@H](O)C[C@H]6C(=O)N[C@@H](C)c6ccc(-c7scnc7C)cc6)C(C)(C)C)CC5)nc4c3F)c12] |
| 46 | 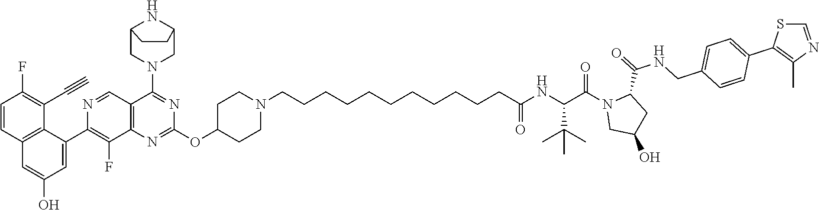 [SMILES: C#Cc1c(F)ccc2cc(O)cc(-c3ncc4c(N5CC6CCC(C5)N6)nc(OC5CCN(CCCCCCCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 47 | 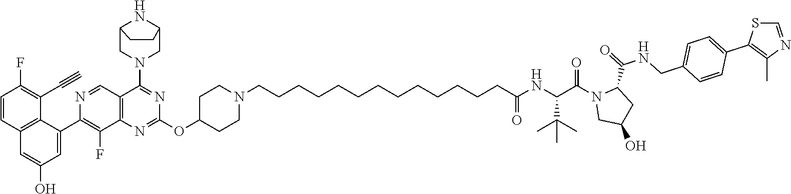 [SMILES: C#Cc1c(F)ccc2cc(O)cc(-c3ncc4c(N5CC6CCC(C5)N6)nc(OC5CCN(CCCCCCCCCCCCCC(=O)N[C@H](C(=O)N6C[C@H](O)C[C@H]6C(=O)NCc6ccc(-c7scnc7C)cc6)C(C)(C)C)CC5)nc4c3F)c12] |
| 48 | 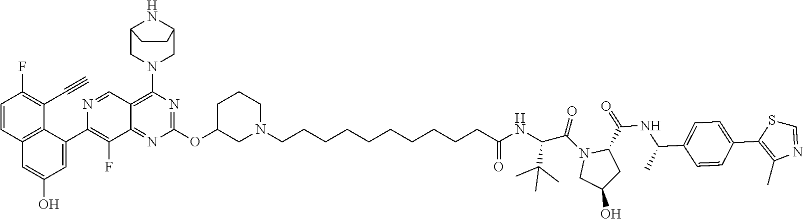 [SMILES: C#Cc1c(F)ccc2cc(O)cc(-c3ncc4c(N5CC6CCC(C5)N6)nc(OC5CCCN(CCCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12] |
| 49 | 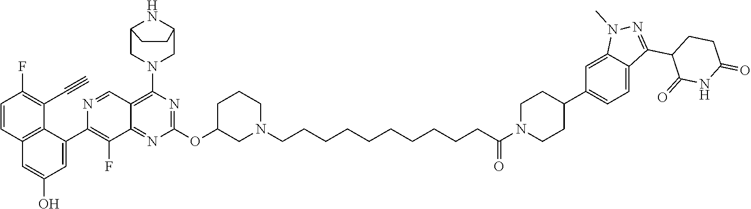 [SMILES: C#Cc1c(F)ccc2cc(O)cc(-c3ncc4c(N5CC6CCC(C5)N6)nc(OC5CCCN(CCCCCCCCCCC(=O)N6CCC(c7ccc8c(C9CCC(=O)NC9=O)nn(C)c8c7)CC6)C5)nc4c3F)c12] |
| 50 | 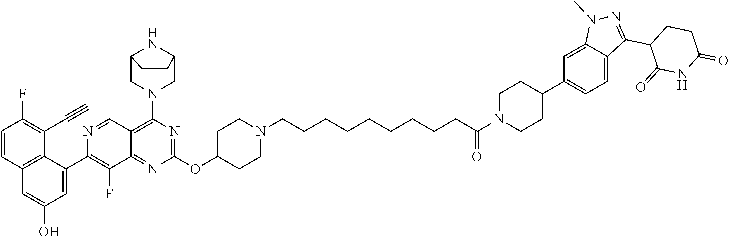 [SMILES: C#Cc1c(F)ccc2cc(O)cc(-c3ncc4c(N5CC6CCC(C5)N6)nc(OC5CCN(CCCCCCCCCC(=O)N6CCC(c7ccc8c(C9CCC(=O)NC9=O)nn(C)c8c7)CC6)CC5)nc4c3F)c12] |
| 51 | 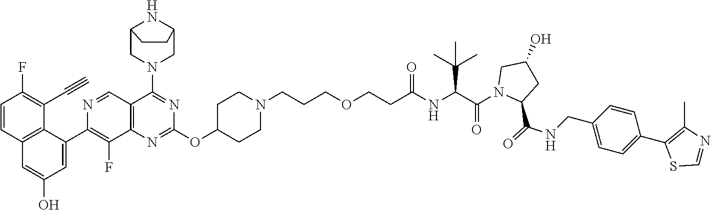 [SMILES: C#Cc1c(F)ccc2cc(O)cc(-c3ncc4c(N5CC6CCC(C5)N6)nc(OC5CCN(CCCOCCC(=O)N[C@H](C(=O)N6C[C@H](O)C[C@H]6C(=O)NCc6ccc(-c7scnc7C)cc6)C(C)(C)C)CC5)nc4c3F)c12] |
| 52 | 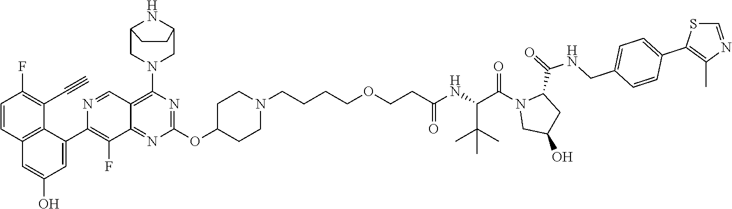 [SMILES: C#Cc1c(F)ccc2cc(O)cc(-c3ncc4c(N5CC6CCC(C5)N6)nc(OC5CCN(CCCCOCCC(=O)N[C@H](C(=O)N6C[C@H](O)C[C@H]6C(=O)NCc6ccc(-c7scnc7C)cc6)C(C)(C)C)CC5)nc4c3F)c12] |
| 53 | 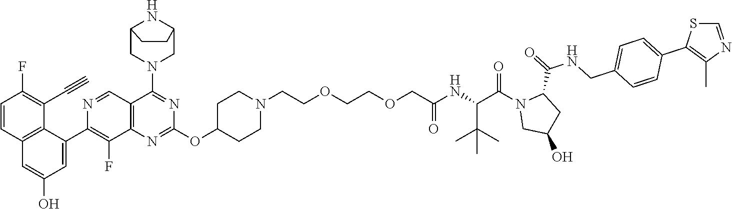 [SMILES: C#Cc1c(F)ccc2cc(O)cc(-c3ncc4c(N5CC6CCC(C5)N6)nc(OC5CCN(CCOCCOCC(=O)N[C@H](C(=O)N6C[C@H](O)C[C@H]6C(=O)NCc6ccc(-c7scnc7C)cc6)C(C)(C)C)CC5)nc4c3F)c12] |
| 54 | 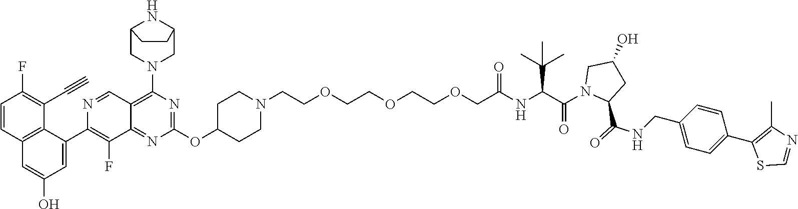 [SMILES: C#Cc1c(F)ccc2cc(O)cc(-c3ncc4c(N5CC6CCC(C5)N6)nc(OC5CCN(CCOCCOCCOCC(=O)N[C@H](C(=O)N6C[C@H](O)C[C@H]6C(=O)NCc6ccc(-c7scnc7C)cc6)C(C)(C)C)CC5)nc4c3F)c12] |
| 55 | 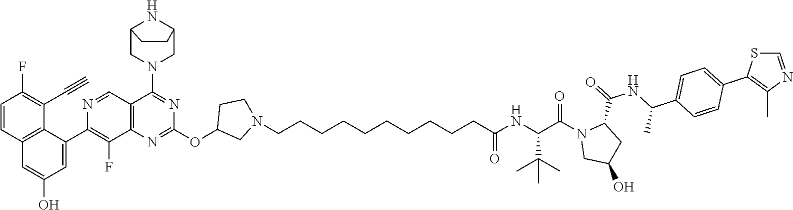 [SMILES: C#Cc1c(F)ccc2cc(O)cc(-c3ncc4c(N5CC6CCC(C5)N6)nc(OC5CCN(CCCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12] |
| 56 | 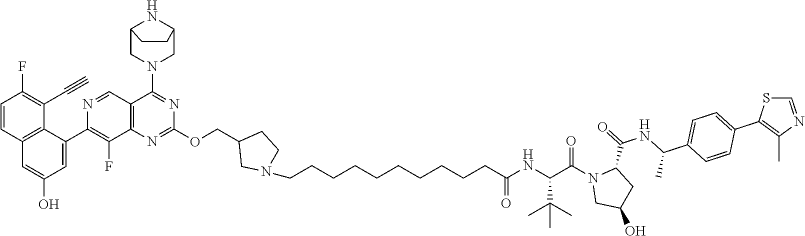 [SMILES: C#Cc1c(F)ccc2cc(O)cc(-c3ncc4c(N5CC6CCC(C5)N6)nc(OCC5CCN(CCCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12] |
| 57 | 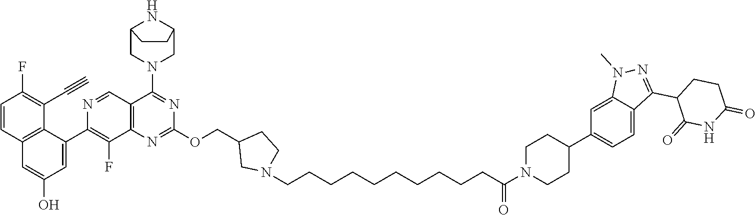 [SMILES: C#Cc1c(F)ccc2cc(O)cc(-c3ncc4c(N5CC6CCC(C5)N6)nc(OCC5CCN(CCCCCCCCCCC(=O)N6CCC(c7ccc8c(C9CCC(=O)NC9=O)nn(C)c8c7)CC6)C5)nc4c3F)c12] |
| 58 | 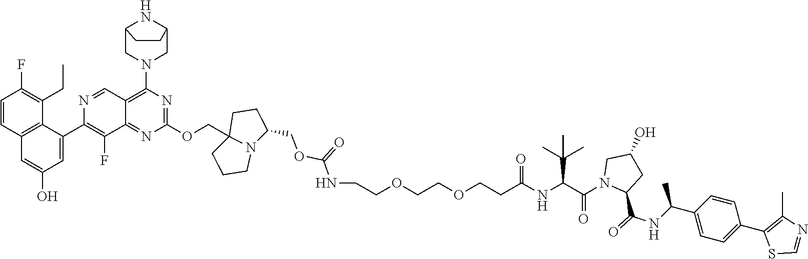 [SMILES: CCc1c(F)ccc2cc(O)cc(-c3ncc4c(N5CC6CCC(C5)N6)nc(OCC56CCCN5[C@@H](COC(=O)NCCOCCOCCC(=O)N[C@H](C(=O)N5C[C@H](O)C[C@H]5C(=O)N[C@@H](C)c5ccc(-c7scnc7C)cc5)C(C)(C)C)CC6)nc4c3F)c12] |
| 59 | 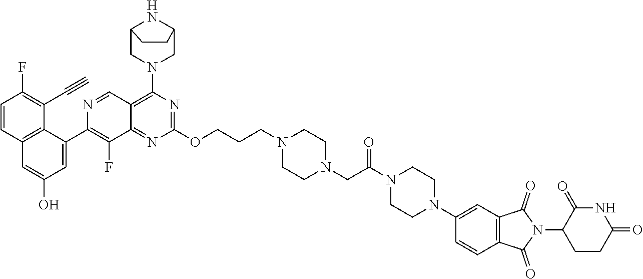 [SMILES: C#Cc1c(F)ccc2cc(O)cc(-c3ncc4c(N5CC6CCC(C5)N6)nc(OCCCN5CCN(CC(=O)N6CCN(c7ccc8c(c7)C(=O)N(C7CCC(=O)NC7=O)C8=O)CC6)CC5)nc4c3F)c12] |
| 60 | 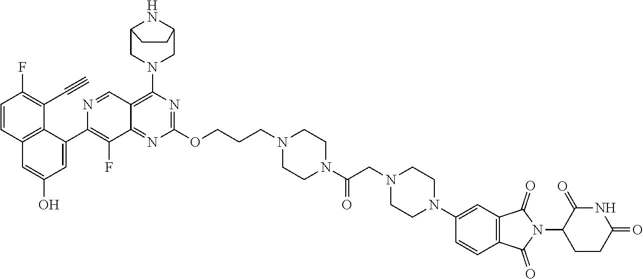 [SMILES: C#Cc1c(F)ccc2cc(O)cc(-c3ncc4c(N5CC6CCC(C5)N6)nc(OCCCN5CCN(C(=O)CN6CCN(c7ccc8c(c7)C(=O)N(C7CCC(=O)NC7=O)C8=O)CC6)CC5)nc4c3F)c12] |
| 61 | 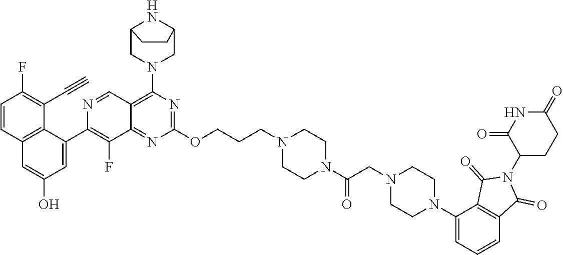 [SMILES: C#Cc1c(F)ccc2cc(O)cc(-c3ncc4c(N5CC6CCC(C5)N6)nc(OCCCN5CCN(C(=O)CN6CCN(c7cccc8c7C(=O)N(C7CCC(=O)NC7=O)C8=O)CC6)CC5)nc4c3F)c12] |
| 62 | 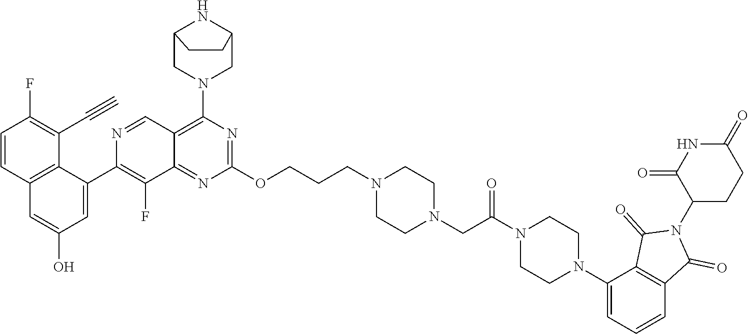 [SMILES: C#Cc1c(F)ccc2cc(O)cc(-c3ncc4c(N5CC6CCC(C5)N6)nc(OCCCN5CCN(CC(=O)N6CCN(c7cccc8c7C(=O)N(C7CCC(=O)NC7=O)C8=O)CC6)CC5)nc4c3F)c12] |
| 63 | 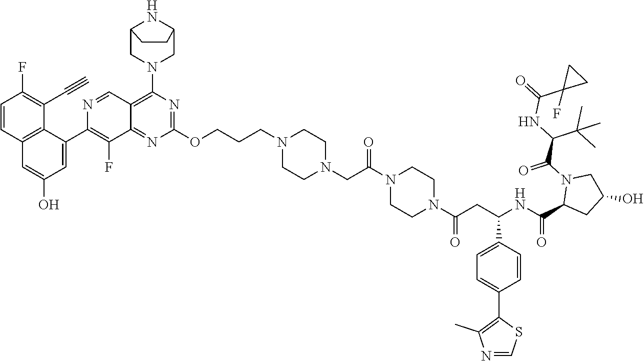 [SMILES: C#Cc1c(F)ccc2cc(O)cc(-c3ncc4c(N5CC6CCC(C5)N6)nc(OCCCN5CCN(CC(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)CC5)nc4c3F)c12] |
| 64 | 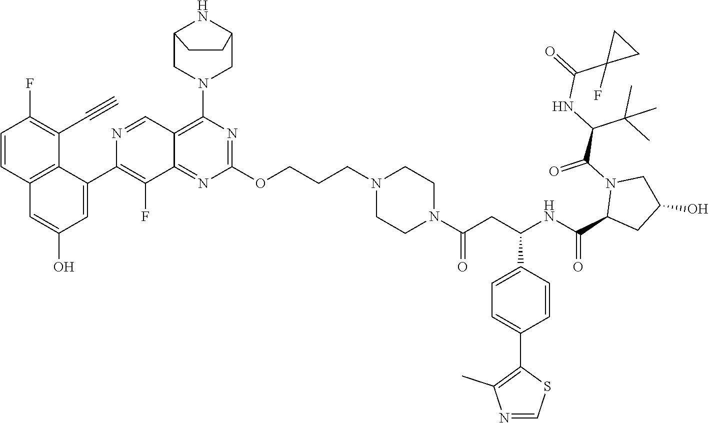 [SMILES: C#Cc1c(F)ccc2cc(O)cc(-c3ncc4c(N5CC6CCC(C5)N6)nc(OCCCN5CCN(C(=O)C[C@H](NC(=O)[C@@H]6C[C@@H](O)CN6C(=O)[C@@H](NC(=O)C6(F)CC6)C(C)(C)C)c6ccc(-c7scnc7C)cc6)CC5)nc4c3F)c12] |
| 65 | 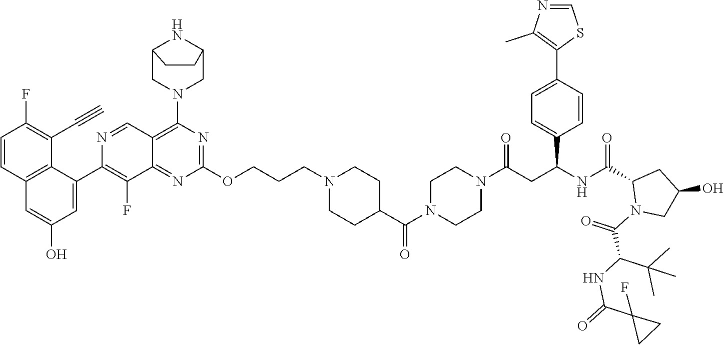 [SMILES: C#Cc1c(F)ccc2cc(O)cc(-c3ncc4c(N5CC6CCC(C5)N6)nc(OCCCN5CCC(C(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)CC5)nc4c3F)c12] |
| 66 | 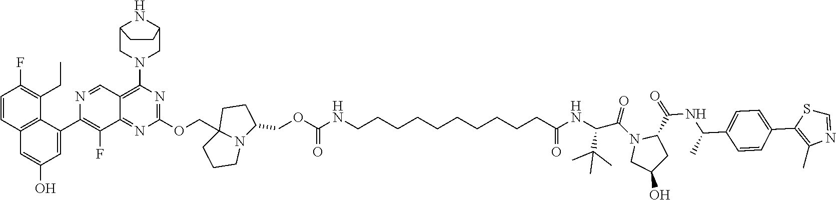 [SMILES: CCc1c(F)ccc2cc(O)cc(-c3ncc4c(N5CC6CCC(C5)N6)nc(OCC56CCCN5[C@@H](COC(=O)NCCCCCCCCCCC(=O)N[C@H](C(=O)N5C[C@H](O)C[C@H]5C(=O)N[C@@H](C)c5ccc(-c7scnc7C)cc5)C(C)(C)C)CC6)nc4c3F)c12] |
| 67 | 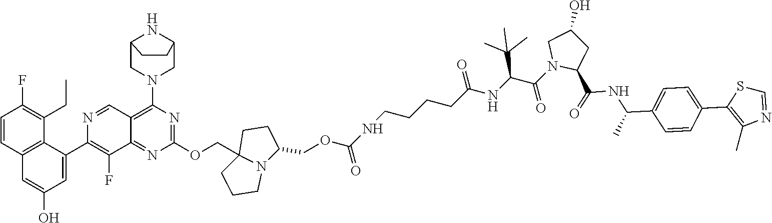 [SMILES: CCc1c(F)ccc2cc(O)cc(-c3ncc4c(N5CC6CCC(C5)N6)nc(OCC56CCCN5[C@@H](COC(=O)NCCCCC(=O)N[C@H](C(=O)N5C[C@H](O)C[C@H]5C(=O)N[C@@H](C)c5ccc(-c7scnc7C)cc5)C(C)(C)C)CC6)nc4c3F)c12] |
| 68 | 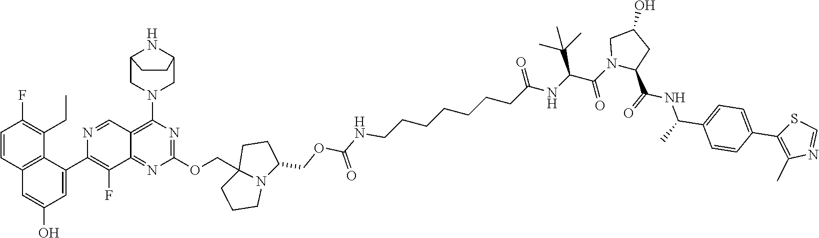 [SMILES: CCc1c(F)ccc2cc(O)cc(-c3ncc4c(N5CC6CCC(C5)N6)nc(OCC56CCCN5[C@@H](COC(=O)NCCCCCCCC(=O)N[C@H](C(=O)N5C[C@H](O)C[C@H]5C(=O)N[C@@H](C)c5ccc(-c7scnc7C)cc5)C(C)(C)C)CC6)nc4c3F)c12] |
| 69 | 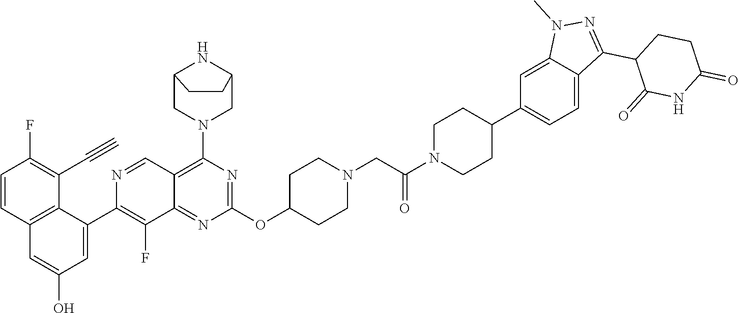 [SMILES: C#Cc1c(F)ccc2cc(O)cc(-c3ncc4c(N5CC6CCC(C5)N6)nc(OC5CCN(CC(=O)N6CCC(c7ccc8c(C9CCC(=O)NC9=O)nn(C)c8c7)CC6)CC5)nc4c3F)c12] |
| 70 | 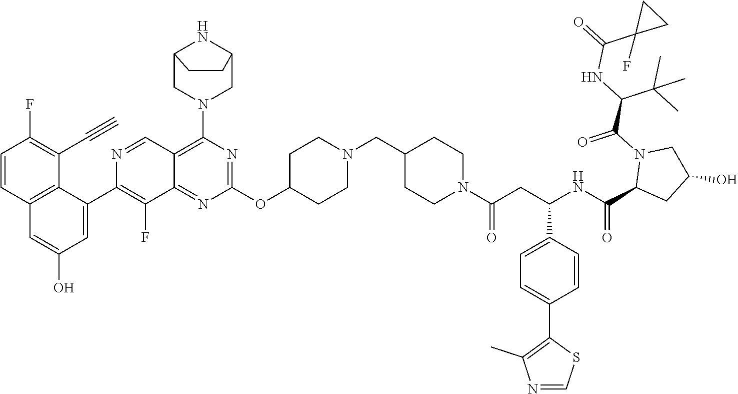 [SMILES: C#Cc1c(F)ccc2cc(O)cc(-c3ncc4c(N5CC6CCC(C5)N6)nc(OC5CCN(CC6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)CC5)nc4c3F)c12] |
| 71 | 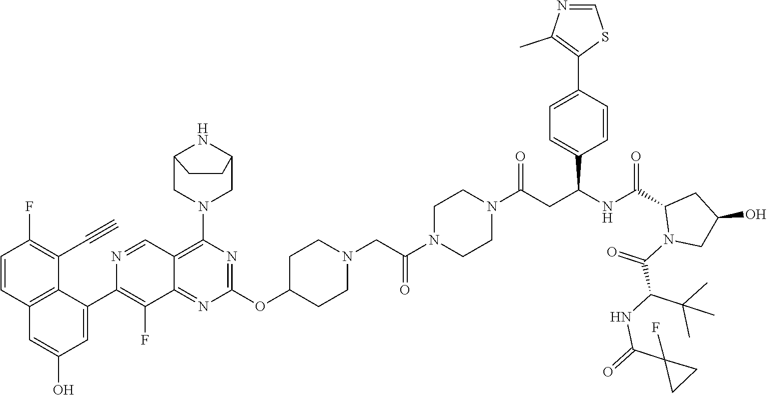 [SMILES: C#Cc1c(F)ccc2cc(O)cc(-c3ncc4c(N5CC6CCC(C5)N6)nc(OC5CCN(CC(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)CC5)nc4c3F)c12] |
| 72 | 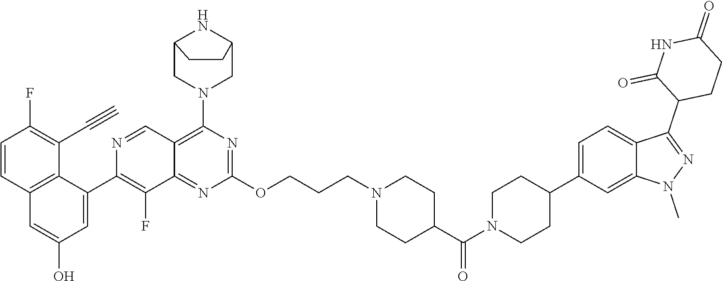 [SMILES: C#Cc1c(F)ccc2cc(O)cc(-c3ncc4c(N5CC6CCC(C5)N6)nc(OCCCN5CCC(C(=O)N6CCC(c7ccc8c(C9CCC(=O)NC9=O)nn(C)c8c7)CC6)CC5)nc4c3F)c12] |
| 73 | 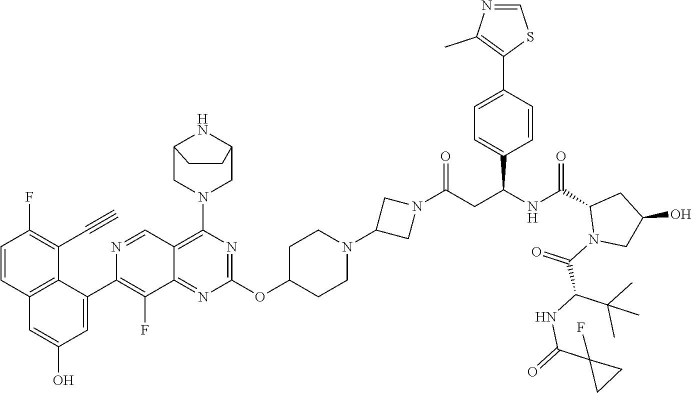 [SMILES: C#Cc1c(F)ccc2cc(O)cc(-c3ncc4c(N5CC6CCC(C5)N6)nc(OC5CCN(C6CN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)C6)CC5)nc4c3F)c12] |
| 74 | 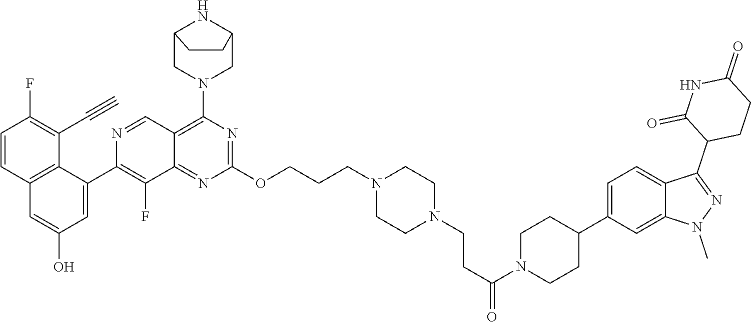 [SMILES: C#Cc1c(F)ccc2cc(O)cc(-c3ncc4c(N5CC6CCC(C5)N6)nc(OCCCN5CCN(CCC(=O)N6CCC(c7ccc8c(C9CCC(=O)NC9=O)nn(C)c8c7)CC6)CC5)nc4c3F)c12] |
| 75 | 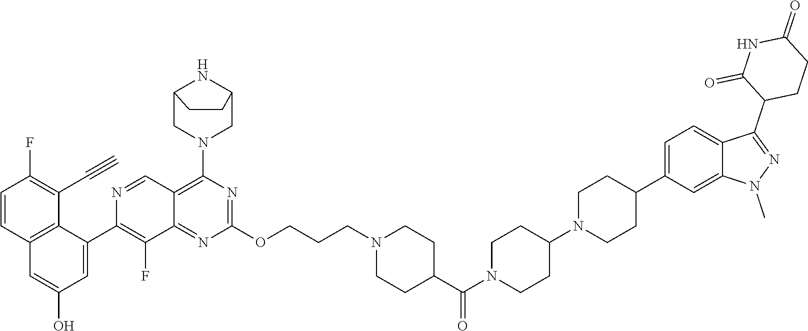 [SMILES: C#Cc1c(F)ccc2cc(O)cc(-c3ncc4c(N5CC6CCC(C5)N6)nc(OCCCN5CCC(C(=O)N6CCC(N7CCC(c8ccc9c(C%10CCC(=O)NC%10=O)nn(C)c9c8)CC7)CC6)CC5)nc4c3F)c12] |
| 76 | 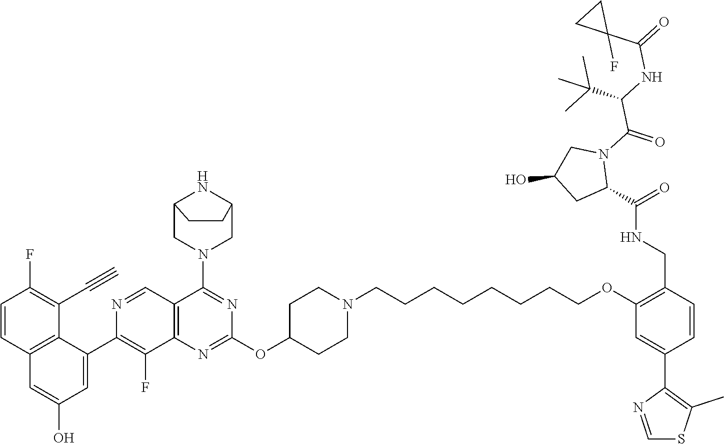 [SMILES: C#Cc1c(F)ccc2cc(O)cc(-c3ncc4c(N5CC6CCC(C5)N6)nc(OC5CCN(CCCCCCCCOc6cc(-c7ncsc7C)ccc6CNC(=O)[C@@H]6C[C@@H](O)CN6C(=O)[C@@H](NC(=O)C6(F)CC6)C(C)(C)C)CC5)nc4c3F)c12] |
| 77 | 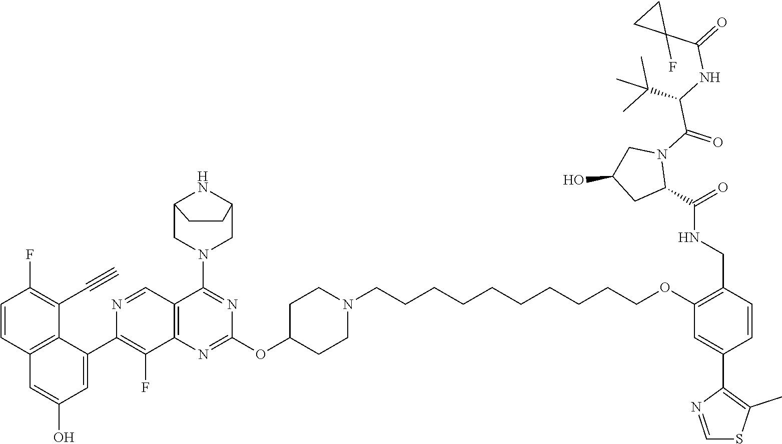 [SMILES: C#Cc1c(F)ccc2cc(O)cc(-c3ncc4c(N5CC6CCC(C5)N6)nc(OC5CCN(CCCCCCCCCCOc6cc(-c7ncsc7C)ccc6CNC(=O)[C@@H]6C[C@@H](O)CN6C(=O)[C@@H](NC(=O)C6(F)CC6)C(C)(C)C)CC5)nc4c3F)c12] |
| 78 | 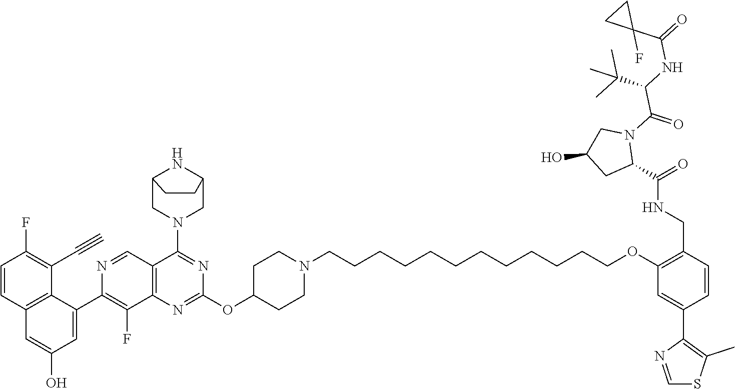 [SMILES: C#Cc1c(F)ccc2cc(O)cc(-c3ncc4c(N5CC6CCC(C5)N6)nc(OC5CCN(CCCCCCCCCCCCOc6cc(-c7ncsc7C)ccc6CNC(=O)[C@@H]6C[C@@H](O)CN6C(=O)[C@@H](NC(=O)C6(F)CC6)C(C)(C)C)CC5)nc4c3F)c12] |
| 79 | 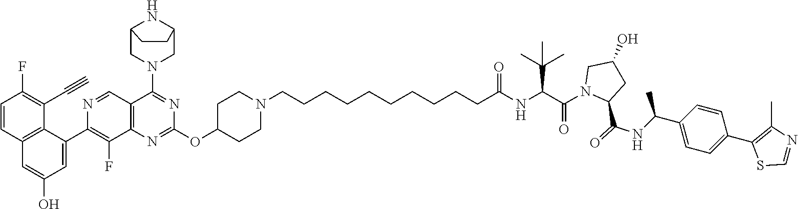 [SMILES: C#Cc1c(F)ccc2cc(O)cc(-c3ncc4c(N5CC6CCC(C5)N6)nc(OC5CCN(CCCCCCCCCCC(=O)N[C@H](C(=O)N6C[C@H](O)C[C@H]6C(=O)N[C@@H](C)c6ccc(-c7scnc7C)cc6)C(C)(C)C)CC5)nc4c3F)c12] |
| 80 | 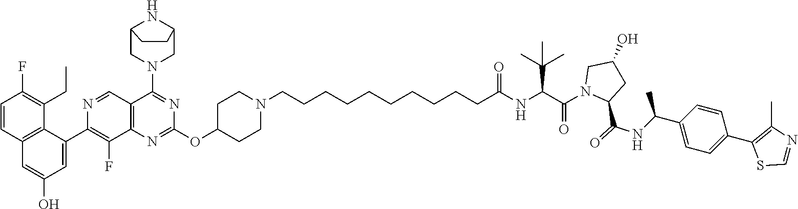 [SMILES: CCc1c(F)ccc2cc(O)cc(-c3ncc4c(N5CC6CCC(C5)N6)nc(OC5CCN(CCCCCCCCCCC(=O)N[C@H](C(=O)N6C[C@H](O)C[C@H]6C(=O)N[C@@H](C)c6ccc(-c7scnc7C)cc6)C(C)(C)C)CC5)nc4c3F)c12] |
| 81 | 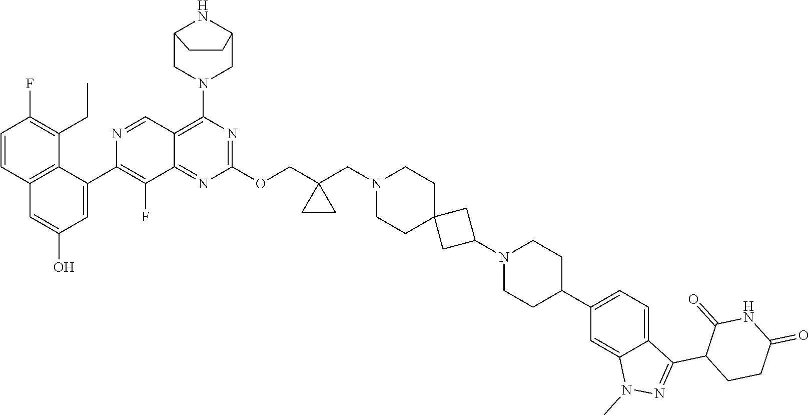 [SMILES: CCc1c(F)ccc2cc(O)cc(-c3ncc4c(N5CC6CCC(C5)N6)nc(OCC5(CN6CCC7(CC6)CC(N6CCC(c8ccc9c(C%10CCC(=O)NC%10=O)nn(C)c9c8)CC6)C7)CC5)nc4c3F)c12] |
| 82 | 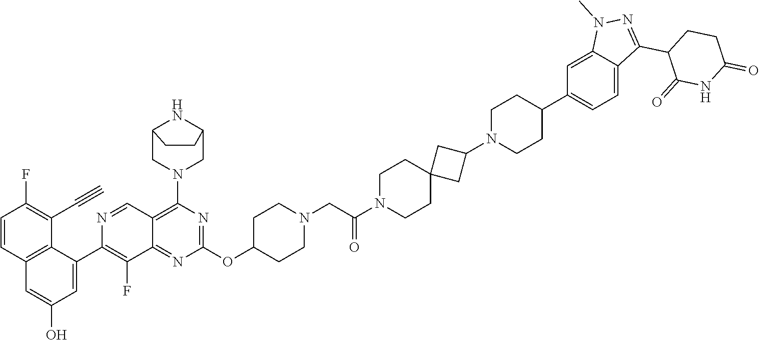 [SMILES: C#Cc1c(F)ccc2cc(O)cc(-c3ncc4c(N5CC6CCC(C5)N6)nc(OC5CCN(CC(=O)N6CCC7(CC6)CC(N6CCC(c8ccc9c(C%10CCC(=O)NC%10=O)nn(C)c9c8)CC6)C7)CC5)nc4c3F)c12] |
| 83 | 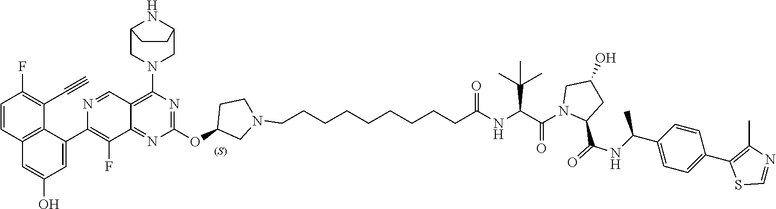 |
| 84 | 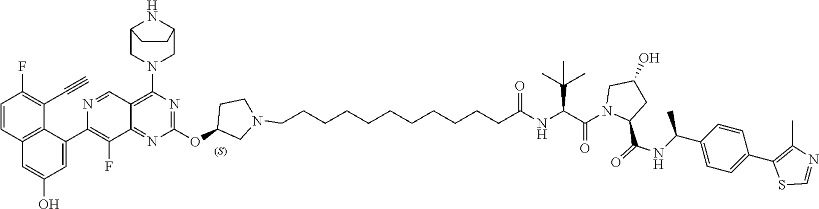 |
| 85 | 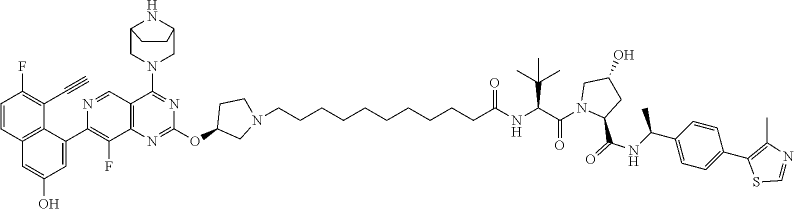 [SMILES: C#Cc1c(F)ccc2cc(O)cc(-c3ncc4c(N5CC6CCC(C5)N6)nc(O[C@H]5CCN(CCCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12] |
| 86 | 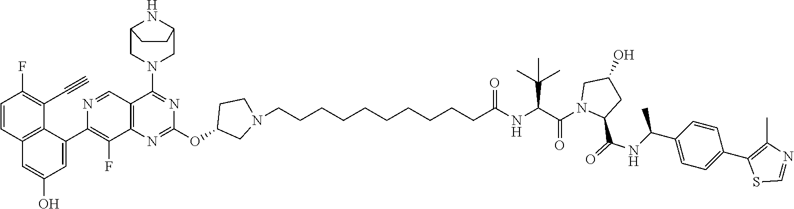 [SMILES: C#Cc1c(F)ccc2cc(O)cc(-c3ncc4c(N5CC6CCC(C5)N6)nc(O[C@@H]5CCN(CCCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12] |
| 87 | 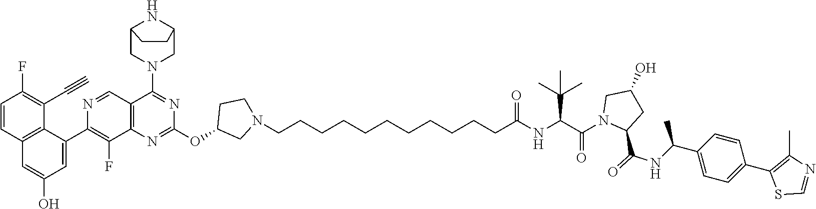 [SMILES: C#Cc1c(F)ccc2cc(O)cc(-c3ncc4c(N5CC6CCC(C5)N6)nc(O[C@@H]5CCN(CCCCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12] |
| 88 | 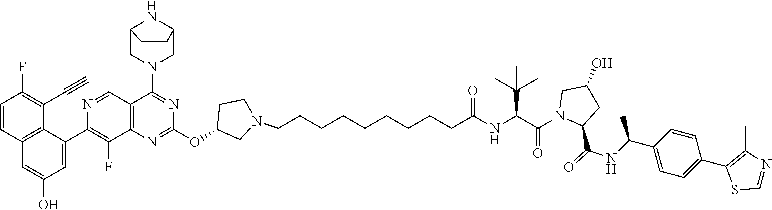 [SMILES: C#Cc1c(F)ccc2cc(O)cc(-c3ncc4c(N5CC6CCC(C5)N6)nc(O[C@@H]5CCN(CCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12] |
| 89 | 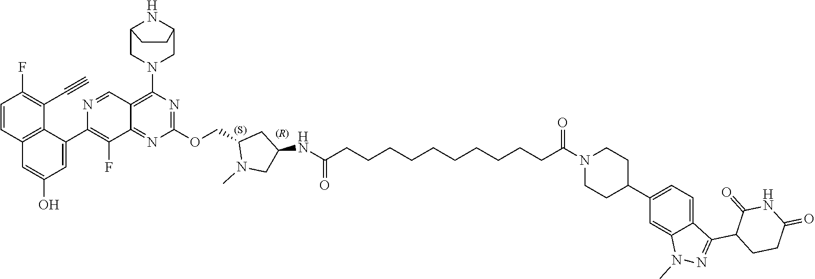 |
| 90 | 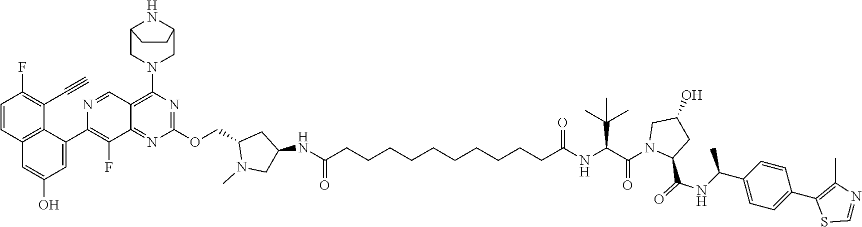 [SMILES: C#Cc1c(F)ccc2cc(O)cc(-c3ncc4c(N5CC6CCC(C5)N6)nc(OC[C@@H]5C[C@@H](NC(=O)CCCCCCCCCCC(=O)N[C@H](C(=O)N6C[C@H](O)C[C@H]6C(=O)N[C@@H](C)c6ccc(-c7scnc7C)cc6)C(C)(C)C)CN5C)nc4c3F)c12] |
| 91 | 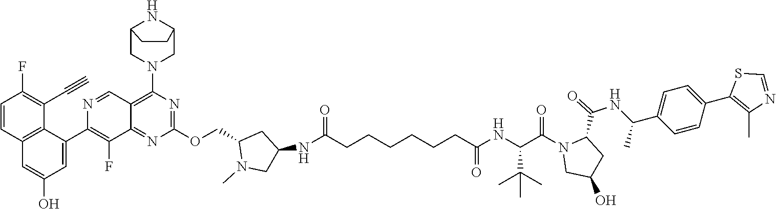 [SMILES: C#Cc1c(F)ccc2cc(O)cc(-c3ncc4c(N5CC6CCC(C5)N6)nc(OC[C@@H]5C[C@@H](NC(=O)CCCCCCC(=O)N[C@H](C(=O)N6C[C@H](O)C[C@H]6C(=O)N[C@@H](C)c6ccc(-c7scnc7C)cc6)C(C)(C)C)CN5C)nc4c3F)c12] |
| 92 | 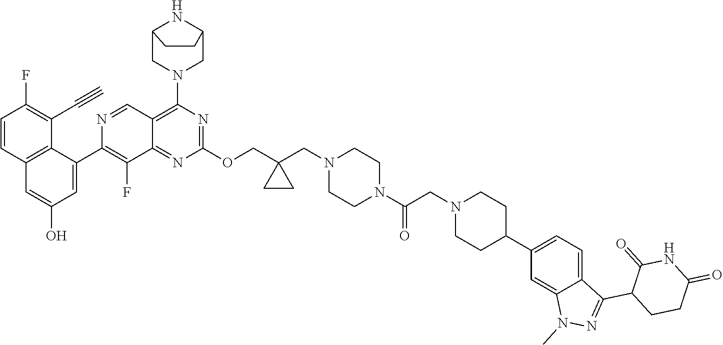 [SMILES: C#Cc1c(F)ccc2cc(O)cc(-c3ncc4c(N5CC6CCC(C5)N6)nc(OCC5(CN6CCN(C(=O)CN7CCC(c8ccc9c(C%10CCC(=O)NC%10=O)nn(C)c9c8)CC7)CC6)CC5)nc4c3F)c12] |
| 93 | 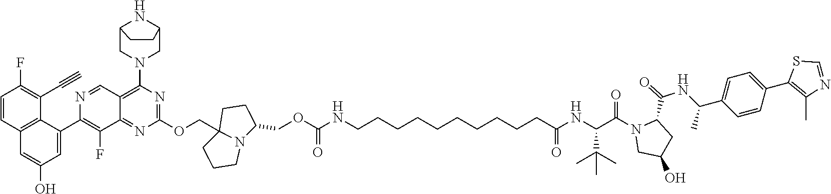 [SMILES: C#Cc1c(F)ccc2cc(O)cc(-c3ncc4c(N5CC6CCC(C5)N6)nc(OCC56CCCN5[C@@H](COC(=O)NCCCCCCCCCCC(=O)N[C@H](C(=O)N5C[C@H](O)C[C@H]5C(=O)N[C@@H](C)c5ccc(-c7scnc7C)cc5)C(C)(C)C)CC6)nc4c3F)c12] |
| 94 | 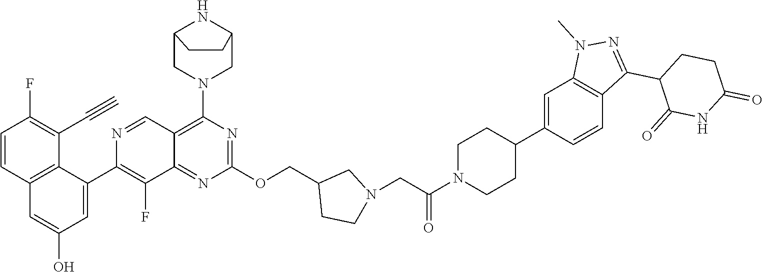 [SMILES: C#Cc1c(F)ccc2cc(O)cc(-c3ncc4c(N5CC6CCC(C5)N6)nc(OCC5CCN(CC(=O)N6CCC(c7ccc8c(C9CCC(=O)NC9=O)nn(C)c8c7)CC6)C5)nc4c3F)c12] |
| 95 | 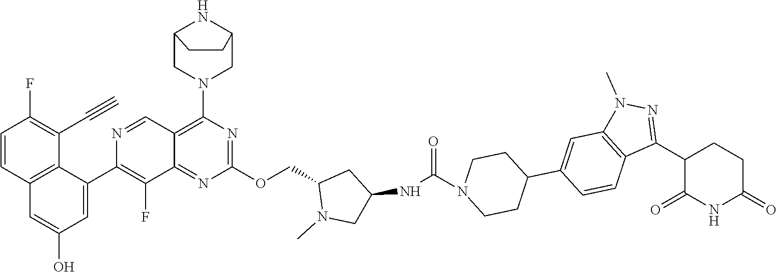 [SMILES: C#Cc1c(F)ccc2cc(O)cc(-c3ncc4c(N5CC6CCC(C5)N6)nc(OC[C@@H]5C[C@@H](NC(=O)N6CCC(c7ccc8c(C9CCC(=O)NC9=O)nn(C)c8c7)CC6)CN5C)nc4c3F)c12] |
| 96 | 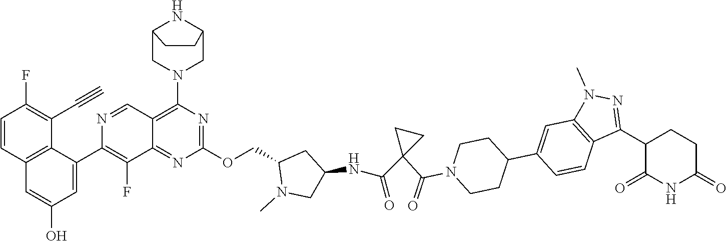 [SMILES: C#Cc1c(F)ccc2cc(O)cc(-c3ncc4c(N5CC6CCC(C5)N6)nc(OC[C@@H]5C[C@@H](NC(=O)C6(C(=O)N7CCC(c8ccc9c(C%10CCC(=O)NC%10=O)nn(C)c9c8)CC7)CC6)CN5C)nc4c3F)c12] |
| 97 | 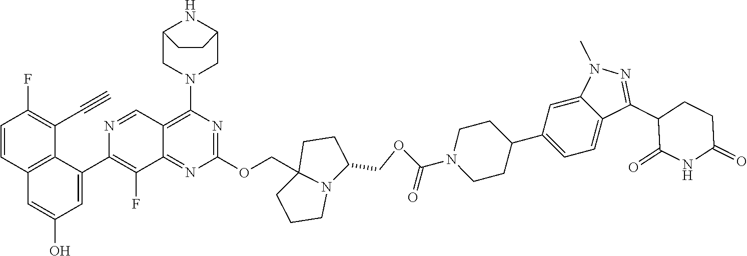 [SMILES: C#Cc1c(F)ccc2cc(O)cc(-c3ncc4c(N5CC6CCC(C5)N6)nc(OCC56CCCN5[C@@H](COC(=O)N5CCC(c7ccc8c(C9CCC(=O)NC9=O)nn(C)c8c7)CC5)CC6)nc4c3F)c12] |
| 98 | 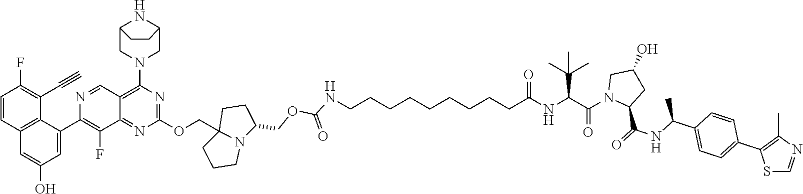 [SMILES: C#Cc1c(F)ccc2cc(O)cc(-c3ncc4c(N5CC6CCC(C5)N6)nc(OCC56CCCN5[C@@H](COC(=O)NCCCCCCCCCC(=O)N[C@H](C(=O)N5C[C@H](O)C[C@H]5C(=O)N[C@@H](C)c5ccc(-c7scnc7C)cc5)C(C)(C)C)CC6)nc4c3F)c12] |
| 99 | 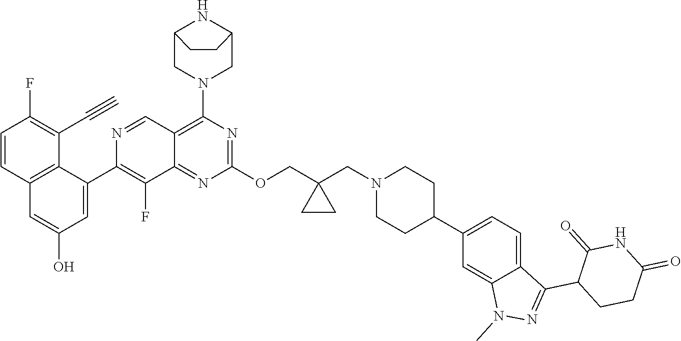 [SMILES: C#Cc1c(F)ccc2cc(O)cc(-c3ncc4c(N5CC6CCC(C5)N6)nc(OCC5(CN6CCC(c7ccc8c(C9CCC(=O)NC9=O)nn(C)c8c7)CC6)CC5)nc4c3F)c12] |
| 100 | 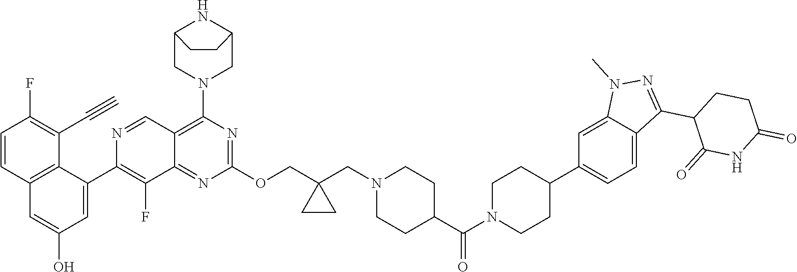 [SMILES: C#Cc1c(F)ccc2cc(O)cc(-c3ncc4c(N5CC6CCC(C5)N6)nc(OCC5(CN6CCC(C(=O)N7CCC(c8ccc9c(C%10CCC(=O)NC%10=O)nn(C)c9c8)CC7)CC6)CC5)nc4c3F)c12] |
| 101 | 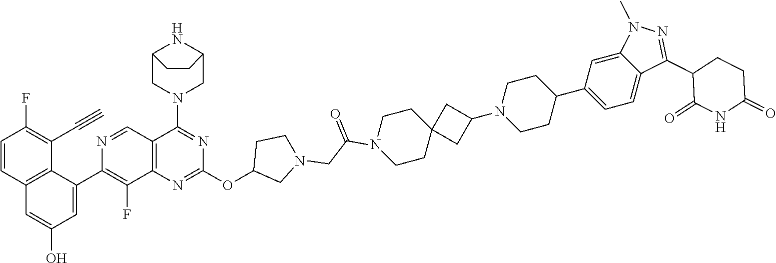 [SMILES: C#Cc1c(F)ccc2cc(O)cc(-c3ncc4c(N5CC6CCC(C5)N6)nc(OC5CCN(CC(=O)N6CCC7(CC6)CC(N6CCC(c8ccc9c(C%10CCC(=O)NC%10=O)nn(C)c9c8)CC6)C7)C5)nc4c3F)c12] |
| 102 | 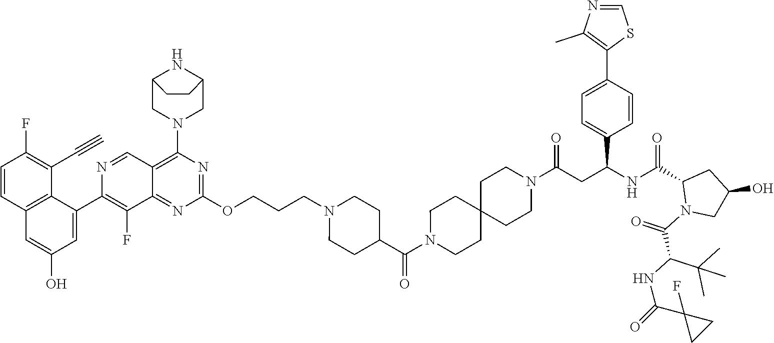 [SMILES: C#Cc1c(F)ccc2cc(O)cc(-c3ncc4c(N5CC6CCC(C5)N6)nc(OCCCN5CCC(C(=O)N6CCC7(CCN(C(=O)C[C@H](NC(=O)[C@@H]8C[C@@H](O)CN8C(=O)[C@@H](NC(=O)C8(F)CC8)C(C)(C)C)c8ccc(-c9scnc9C)cc8)CC7)CC6)CC5)nc4c3F)c12] |
| 103 | 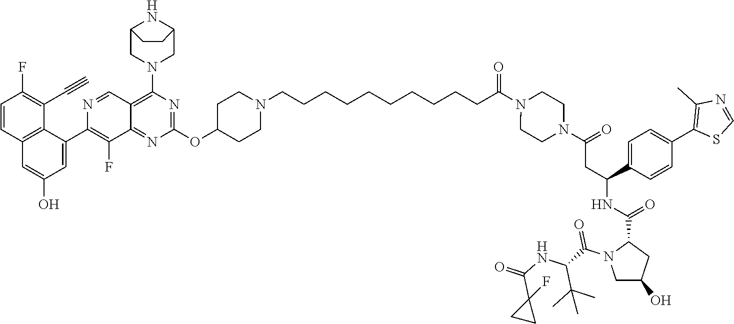 [SMILES: C#Cc1c(F)ccc2cc(O)cc(-c3ncc4c(N5CC6CCC(C5)N6)nc(OC5CCN(CCCCCCCCCCC(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)CC5)nc4c3F)c12] |
| 104 | 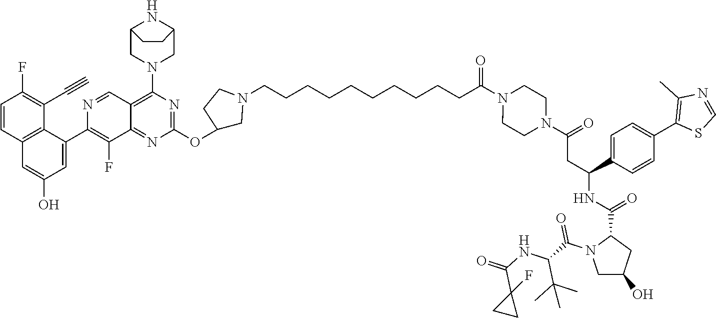 [SMILES: C#Cc1c(F)ccc2cc(O)cc(-c3ncc4c(N5CC6CCC(C5)N6)nc(OC5CCN(CCCCCCCCCCC(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)C5)nc4c3F)c12] |
| 105 | 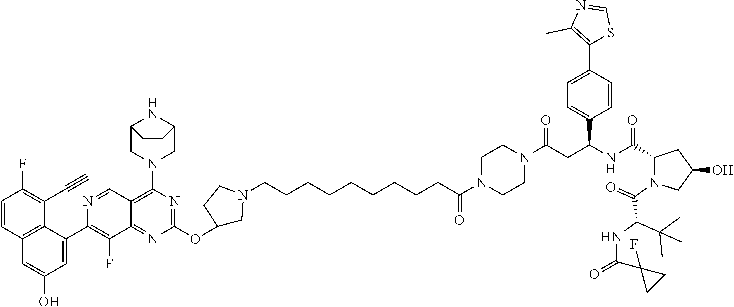 [SMILES: C#Cc1c(F)ccc2cc(O)cc(-c3ncc4c(N5CC6CCC(C5)N6)nc(OC5CCN(CCCCCCCCCC(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)C5)nc4c3F)c12] |
| 106 | 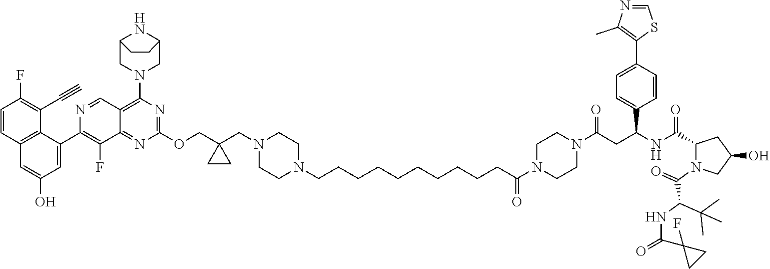 [SMILES: C#Cc1c(F)ccc2cc(O)cc(-c3ncc4c(N5CC6CCC(C5)N6)nc(OCC5(CN6CCN(CCCCCCCCCCC(=O)N7CCN(C(=O)C[C@H](NC(=O)[C@@H]8C[C@@H](O)CN8C(=O)[C@@H](NC(=O)C8(F)CC8)C(C)(C)C)c8ccc(-c9scnc9C)cc8)CC7)CC6)CC5)nc4c3F)c12] |
| 107 | 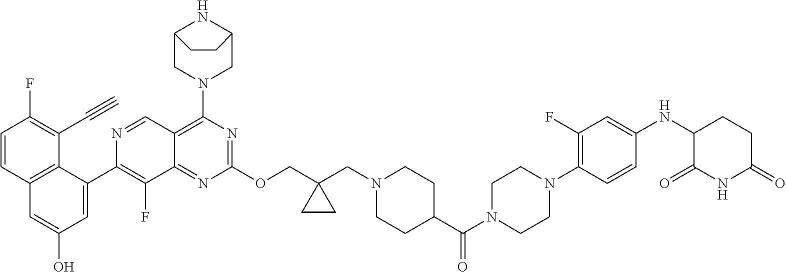 [SMILES: C#Cc1c(F)ccc2cc(O)cc(-c3ncc4c(N5CC6CCC(C5)N6)nc(OCC5(CN6CCC(C(=O)N7CCN(c8ccc(NC9CCC(=O)NC9=O)cc8F)CC7)CC6)CC5)nc4c3F)c12] |
| 108 | 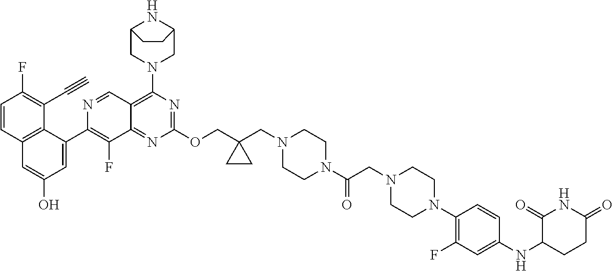 [SMILES: C#Cc1c(F)ccc2cc(O)cc(-c3ncc4c(N5CC6CCC(C5)N6)nc(OCC5(CN6CCN(C(=O)CN7CCN(c8ccc(NC9CCC(=O)NC9=O)cc8F)CC7)CC6)CC5)nc4c3F)c12] |
| 109 | 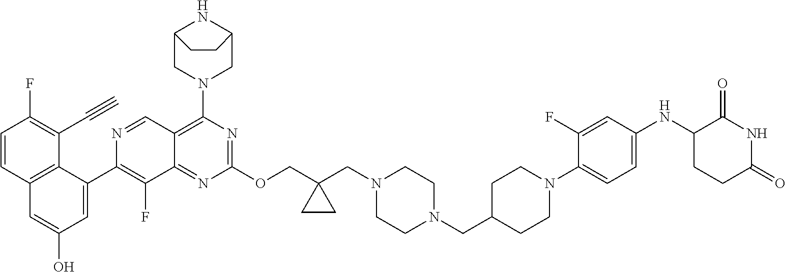 [SMILES: C#Cc1c(F)ccc2cc(O)cc(-c3ncc4c(N5CC6CCC(C5)N6)nc(OCC5(CN6CCN(CC7CCN(c8ccc(NC9CCC(=O)NC9=O)cc8F)CC7)CC6)CC5)nc4c3F)c12] |
| 110 | 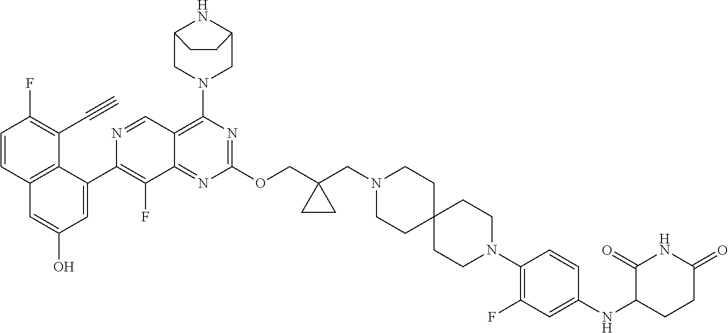 [SMILES: C#Cc1c(F)ccc2cc(O)cc(-c3ncc4c(N5CC6CCC(C5)N6)nc(OCC5(CN6CCC7(CC6)CCN(c6ccc(NC8CCC(=O)NC8=O)cc6F)CC7)CC5)nc4c3F)c12] |
| 111 | 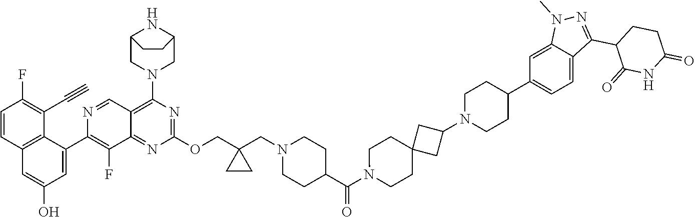 [SMILES: C#Cc1c(F)ccc2cc(O)cc(-c3ncc4c(N5CC6CCC(C5)N6)nc(OCC5(CN6CCC(C(=O)N7CCC8(CC7)CC(N7CCC(c9ccc%10c(C%11CCC(=O)NC%11=O)nn(C)c%10c9)CC7)C8)CC6)CC5)nc4c3F)c12] |
| 112 | 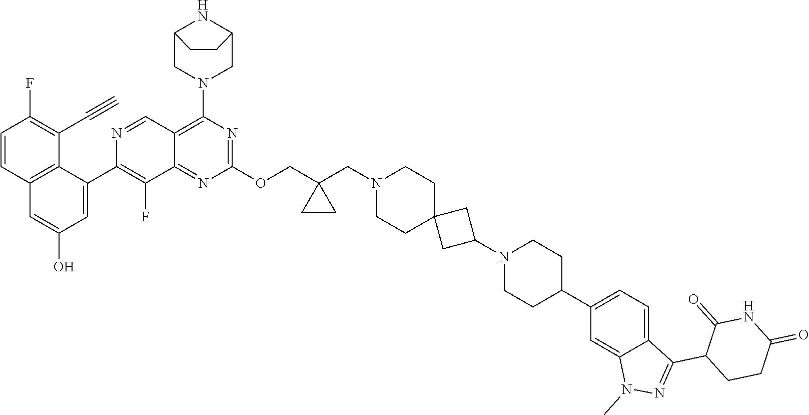 [SMILES: C#Cc1c(F)ccc2cc(O)cc(-c3ncc4c(N5CC6CCC(C5)N6)nc(OCC5(CN6CCC7(CC6)CC(N6CCC(c8ccc9c(C%10CCC(=O)NC%10=O)nn(C)c9c8)CC6)C7)CC5)nc4c3F)c12] |
| 113 | 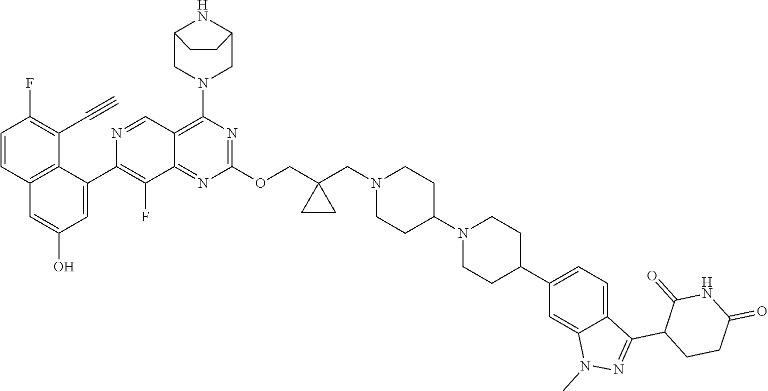 [SMILES: C#Cc1c(F)ccc2cc(O)cc(-c3ncc4c(N5CC6CCC(C5)N6)nc(OCC5(CN6CCC(N7CCC(c8ccc9c(C%10CCC(=O)NC%10=O)nn(C)c9c8)CC7)CC6)CC5)nc4c3F)c12] |
| 114 | 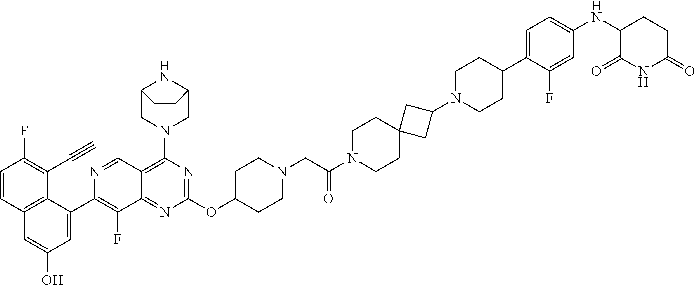 [SMILES: C#Cc1c(F)ccc2cc(O)cc(-c3ncc4c(N5CC6CCC(C5)N6)nc(OC5CCN(CC(=O)N6CCC7(CC6)CC(N6CCC(c8ccc(NC9CCC(=O)NC9=O)cc8F)CC6)C7)CC5)nc4c3F)c12] |
| 115 | 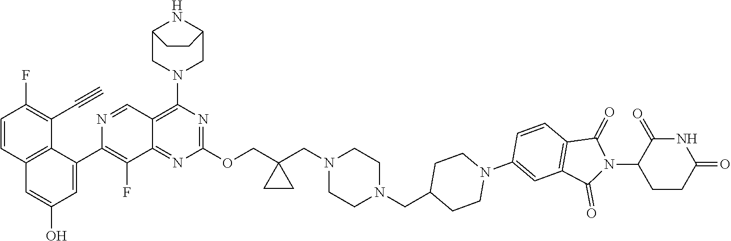 [SMILES: C#Cc1c(F)ccc2cc(O)cc(-c3ncc4c(N5CC6CCC(C5)N6)nc(OCC5(CN6CCN(CC7CCN(c8ccc9c(c8)C(=O)N(C8CCC(=O)NC8=O)C9=O)CC7)CC6)CC5)nc4c3F)c12] |
| 116 | 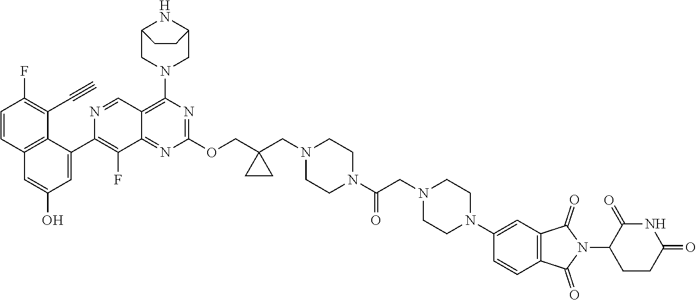 [SMILES: C#Cc1c(F)ccc2cc(O)cc(-c3ncc4c(N5CC6CCC(C5)N6)nc(OCC5(CN6CCN(C(=O)CN7CCN(c8ccc9c(c8)C(=O)N(C8CCC(=O)NC8=O)C9=O)CC7)CC6)CC5)nc4c3F)c12] |
| 117 | 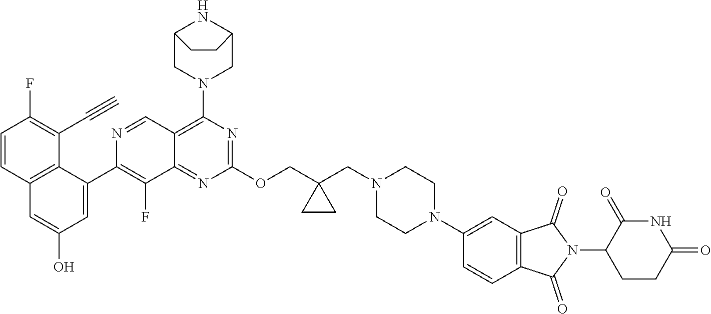 [SMILES: C#Cc1c(F)ccc2cc(O)cc(-c3ncc4c(N5CC6CCC(C5)N6)nc(OCC5(CN6CCN(c7ccc8c(c7)C(=O)N(C7CCC(=O)NC7=O)C8=O)CC6)CC5)nc4c3F)c12] |
| 118 | 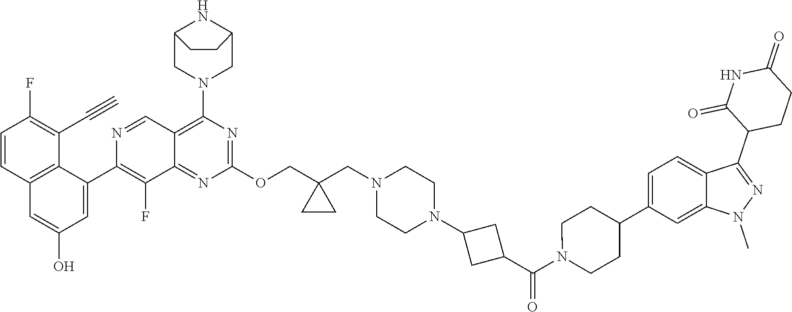 [SMILES: C#Cc1c(F)ccc2cc(O)cc(-c3ncc4c(N5CC6CCC(C5)N6)nc(OCC5(CN6CCN(C7CC(C(=O)N8CCC(c9ccc%10c(C%11CCC(=O)NC%11=O)nn(C)c%10c9)CC8)C7)CC6)CC5)nc4c3F)c12] |
| 119 | 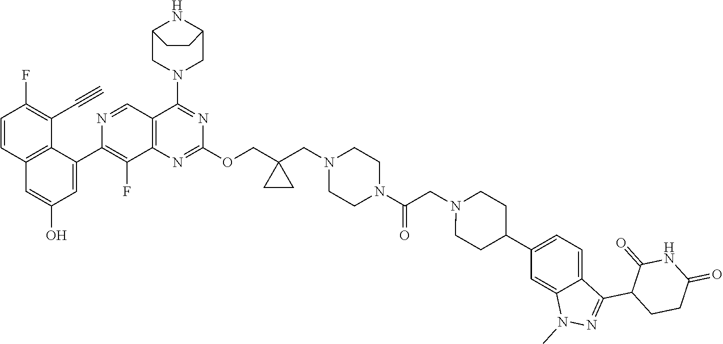 [SMILES: C#Cc1c(F)ccc2cc(O)cc(-c3ncc4c(N5CC6CCC(C5)N6)nc(OCC5(CN6CCN(C(=O)CN7CCC(c8ccc9c(C%10CCC(=O)NC%10=O)nn(C)c9c8)CC7)CC6)CC5)nc4c3F)c12] |
| 120 | 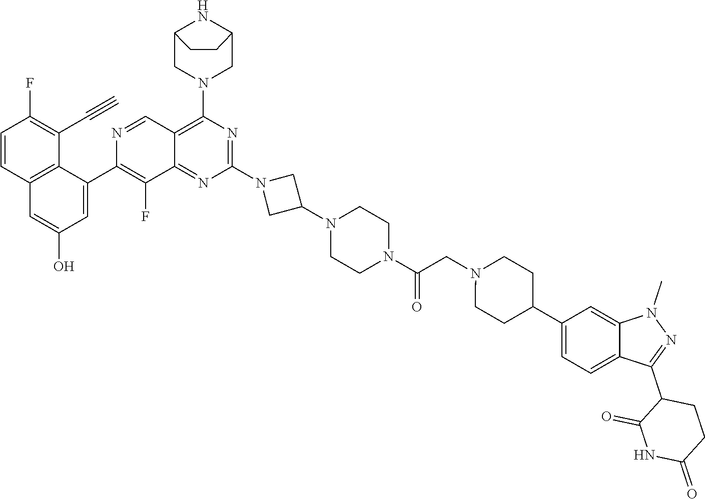 [SMILES: C#Cc1c(F)ccc2cc(O)cc(-c3ncc4c(N5CC6CCC(C5)N6)nc(N5CC(N6CCN(C(=O)CN7CCC(c8ccc9c(C%10CCC(=O)NC%10=O)nn(C)c9c8)CC7)CC6)C5)nc4c3F)c12] |
| 121 | 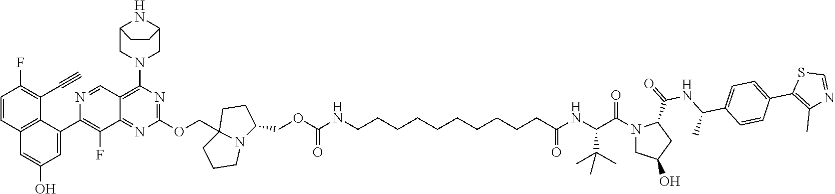 [SMILES: C#Cc1c(F)ccc2cc(O)cc(-c3ncc4c(N5CC6CCC(C5)N6)nc(OCC56CCCN5[C@@H](COC(=O)NCCCCCCCCCCC(=O)N[C@H](C(=O)N5C[C@H](O)C[C@H]5C(=O)N[C@@H](C)c5ccc(-c7scnc7C)cc5)C(C)(C)C)CC6)nc4c3F)c12] |
| 122 | 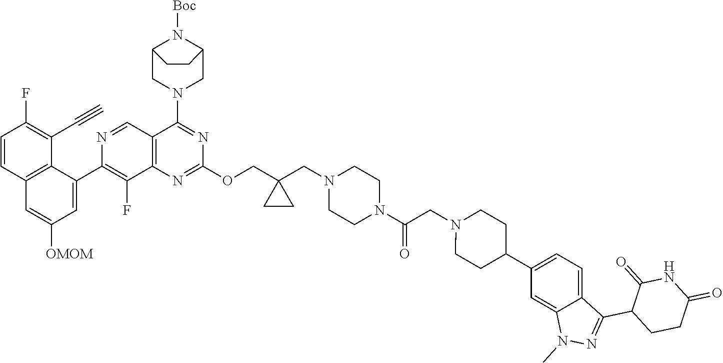 |
| 123 | 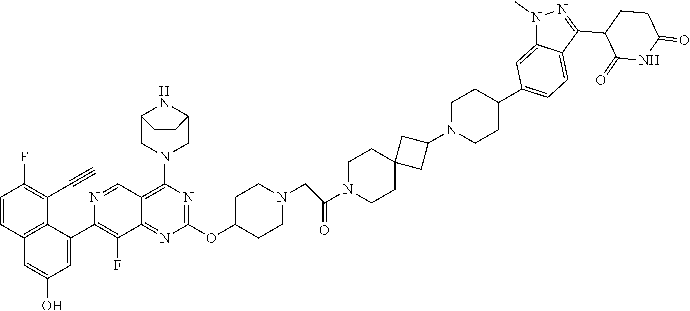 [SMILES: C#Cc1c(F)ccc2cc(O)cc(-c3ncc4c(N5CC6CCC(C5)N6)nc(OC5CCN(CC(=O)N6CCC7(CC6)CC(N6CCC(c8ccc9c(C%10CCC(=O)NC%10=O)nn(C)c9c8)CC6)C7)CC5)nc4c3F)c12] |
| 124 | 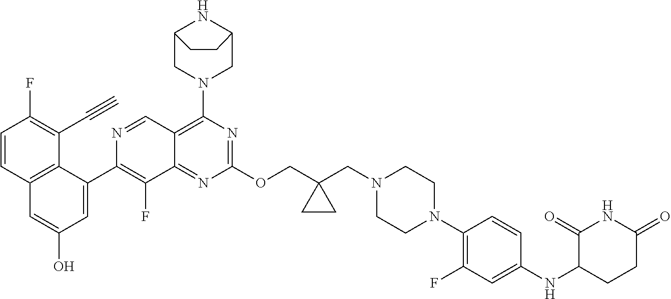 [SMILES: C#Cc1c(F)ccc2cc(O)cc(-c3ncc4c(N5CC6CCC(C5)N6)nc(OCC5(CN6CCN(c7ccc(NC8CCC(=O)NC8=O)cc7F)CC6)CC5)nc4c3F)c12] |
| 125 | 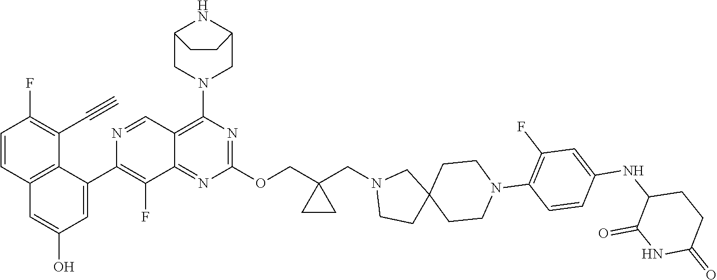 [SMILES: C#Cc1c(F)ccc2cc(O)cc(-c3ncc4c(N5CC6CCC(C5)N6)nc(OCC5(CN6CCC7(CCN(c8ccc(NC9CCC(=O)NC9=O)cc8F)CC7)C6)CC5)nc4c3F)c12] |
| 126 | 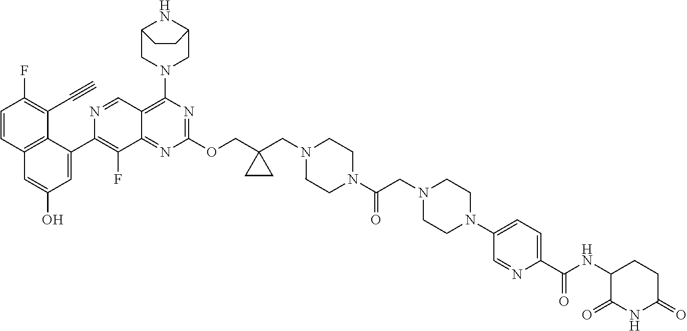 [SMILES: C#Cc1c(F)ccc2cc(O)cc(-c3ncc4c(N5CC6CCC(C5)N6)nc(OCC5(CN6CCN(C(=O)CN7CCN(c8ccc(C(=O)NC9CCC(=O)NC9=O)nc8)CC7)CC6)CC5)nc4c3F)c12] |
| 127 | 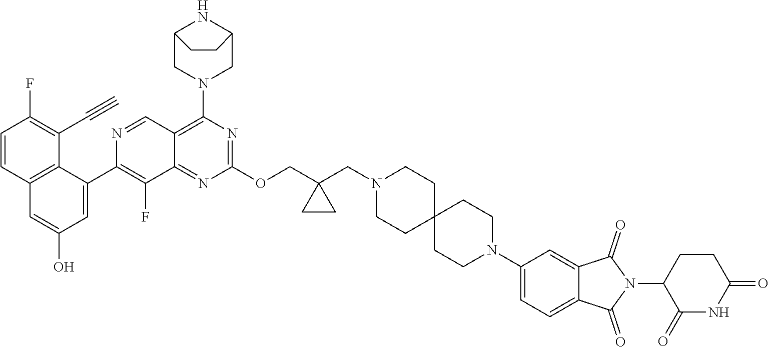 [SMILES: C#Cc1c(F)ccc2cc(O)cc(-c3ncc4c(N5CC6CCC(C5)N6)nc(OCC5(CN6CCC7(CC6)CCN(c6ccc8c(c6)C(=O)N(C6CCC(=O)NC6=O)C8=O)CC7)CC5)nc4c3F)c12] |
| 128 | 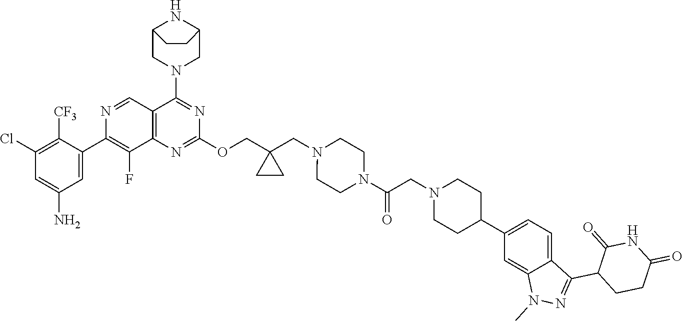 [SMILES: Cn1nc(C2CCC(=O)NC2=O)c2ccc(C3CCN(CC(=O)N4CCN(CC5(COc6nc(N7CC8CCC(C7)N8)c7cnc(-c8cc(N)cc(Cl)c8C(F)(F)F)c(F)c7n6)CC5)CC4)CC3)cc21] |
| 129 | 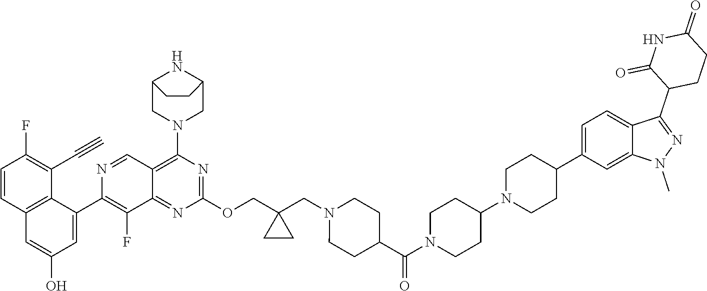 [SMILES: C#Cc1c(F)ccc2cc(O)cc(-c3ncc4c(N5CC6CCC(C5)N6)nc(OCC5(CN6CCC(C(=O)N7CCC(N8CCC(c9ccc%10c(C%11CCC(=O)NC%11=O)nn(C)c%10c9)CC8)CC7)CC6)CC5)nc4c3F)c12] |
| 130 | 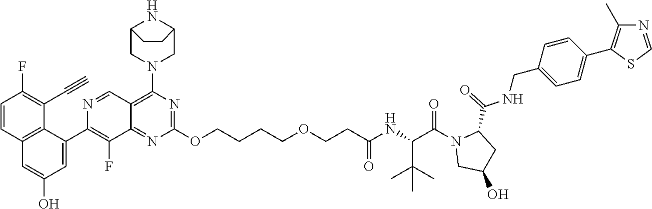 [SMILES: C#Cc1c(F)ccc2cc(O)cc(-c3ncc4c(N5CC6CCC(C5)N6)nc(OCCCCOCCC(=O)N[C@H](C(=O)N5C[C@H](O)C[C@H]5C(=O)NCc5ccc(-c6scnc6C)cc5)C(C)(C)C)nc4c3F)c12] |
| 131 | 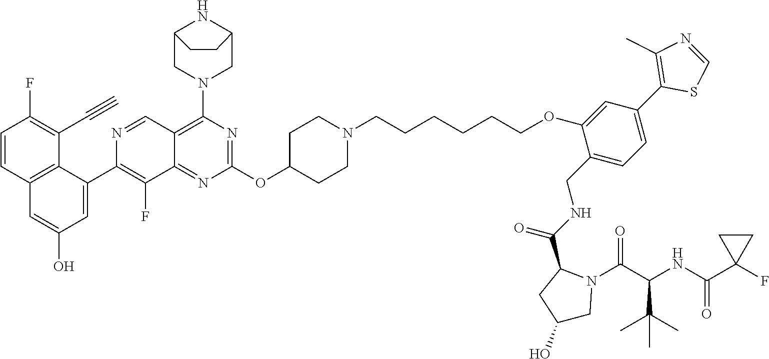 [SMILES: C#Cc1c(F)ccc2cc(O)cc(-c3ncc4c(N5CC6CCC(C5)N6)nc(OC5CCN(CCCCCCOc6cc(-c7scnc7C)ccc6CNC(=O)[C@@H]6C[C@@H](O)CN6C(=O)[C@@H](NC(=O)C6(F)CC6)C(C)(C)C)CC5)nc4c3F)c12] |
| 132 | 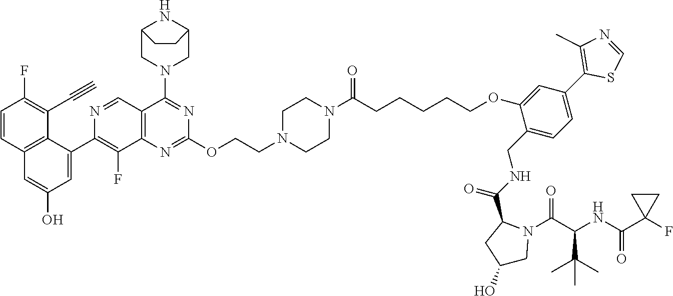 [SMILES: C#Cc1c(F)ccc2cc(O)cc(-c3ncc4c(N5CC6CCC(C5)N6)nc(OCCN5CCN(C(=O)CCCCCOc6cc(-c7scnc7C)ccc6CNC(=O)[C@@H]6C[C@@H](O)CN6C(=O)[C@@H](NC(=O)C6(F)CC6)C(C)(C)C)CC5)nc4c3F)c12] |
| 133 | 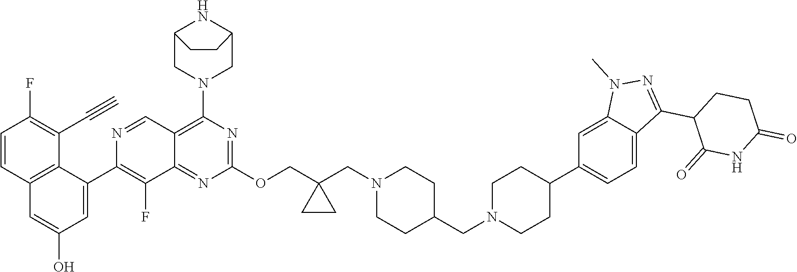 [SMILES: C#Cc1c(F)ccc2cc(O)cc(-c3ncc4c(N5CC6CCC(C5)N6)nc(OCC5(CN6CCC(CN7CCC(c8ccc9c(C%10CCC(=O)NC%10=O)nn(C)c9c8)CC7)CC6)CC5)nc4c3F)c12] |
| 134 | 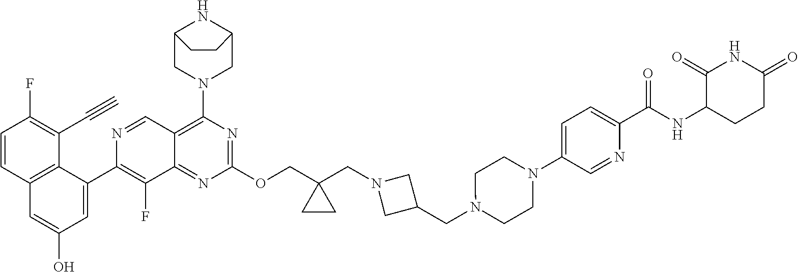 [SMILES: C#Cc1c(F)ccc2cc(O)cc(-c3ncc4c(N5CC6CCC(C5)N6)nc(OCC5(CN6CC(CN7CCN(c8ccc(C(=O)NC9CCC(=O)NC9=O)nc8)CC7)C6)CC5)nc4c3F)c12] |
| 135 | 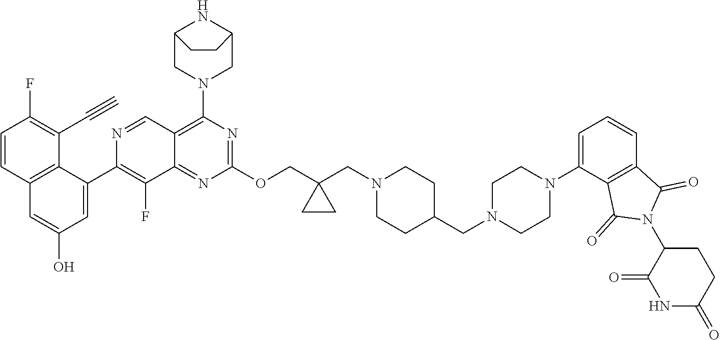 [SMILES: C#Cc1c(F)ccc2cc(O)cc(-c3ncc4c(N5CC6CCC(C5)N6)nc(OCC5(CN6CCC(CN7CCN(c8cccc9c8C(=O)N(C8CCC(=O)NC8=O)C9=O)CC7)CC6)CC5)nc4c3F)c12] |
| 136 | 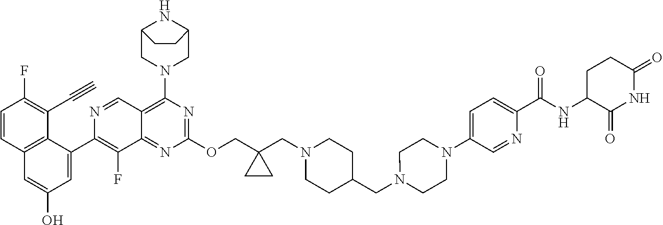 [SMILES: C#Cc1c(F)ccc2cc(O)cc(-c3ncc4c(N5CC6CCC(C5)N6)nc(OCC5(CN6CCC(CN7CCN(c8ccc(C(=O)NC9CCC(=O)NC9=O)nc8)CC7)CC6)CC5)nc4c3F)c12] |
| 137 | 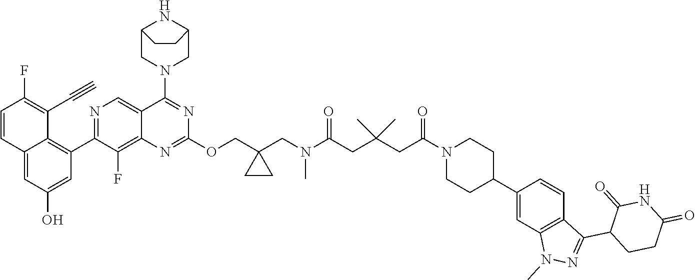 [SMILES: C#Cc1c(F)ccc2cc(O)cc(-c3ncc4c(N5CC6CCC(C5)N6)nc(OCC5(CN(C)C(=O)CC(C)(C)CC(=O)N6CCC(c7ccc8c(C9CCC(=O)NC9=O)nn(C)c8c7)CC6)CC5)nc4c3F)c12] |
| 138 | 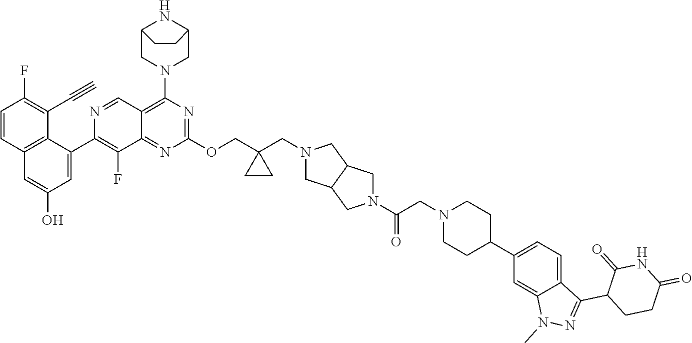 [SMILES: C#Cc1c(F)ccc2cc(O)cc(-c3ncc4c(N5CC6CCC(C5)N6)nc(OCC5(CN6CC7CN(C(=O)CN8CCC(c9ccc%10c(C%11CCC(=O)NC%11=O)nn(C)c%10c9)CC8)CC7C6)CC5)nc4c3F)c12] |
| 139 | 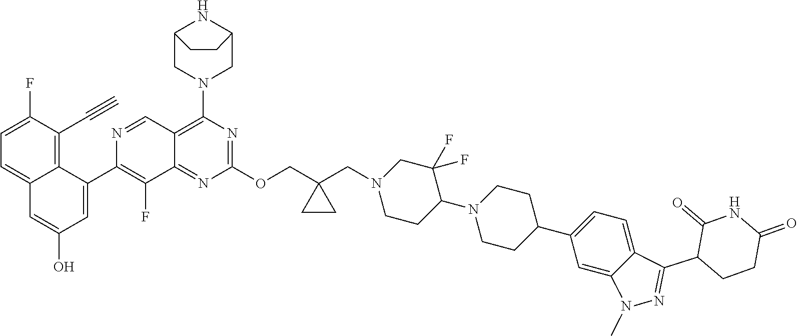 [SMILES: C#Cc1c(F)ccc2cc(O)cc(-c3ncc4c(N5CC6CCC(C5)N6)nc(OCC5(CN6CCC(N7CCC(c8ccc9c(C%10CCC(=O)NC%10=O)nn(C)c9c8)CC7)C(F)(F)C6)CC5)nc4c3F)c12] |
| 140 | 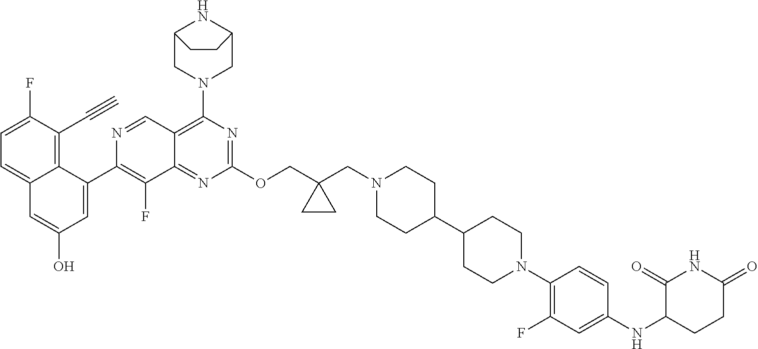 [SMILES: C#Cc1c(F)ccc2cc(O)cc(-c3ncc4c(N5CC6CCC(C5)N6)nc(OCC5(CN6CCC(C7CCN(c8ccc(NC9CCC(=O)NC9=O)cc8F)CC7)CC6)CC5)nc4c3F)c12] |
| 141 | 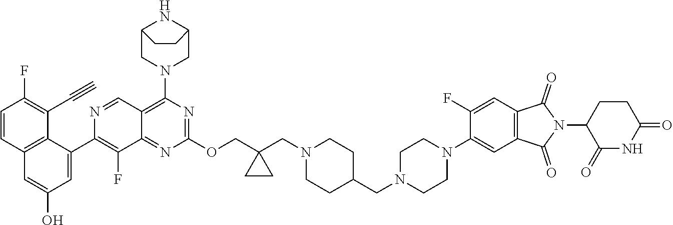 [SMILES: C#Cc1c(F)ccc2cc(O)cc(-c3ncc4c(N5CC6CCC(C5)N6)nc(OCC5(CN6CCC(CN7CCN(c8cc9c(cc8F)C(=O)N(C8CCC(=O)NC8=O)C9=O)CC7)CC6)CC5)nc4c3F)c12] |
| 142 | 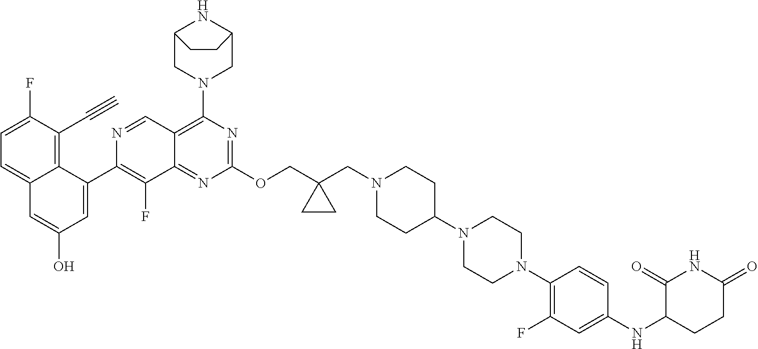 [SMILES: C#Cc1c(F)ccc2cc(O)cc(-c3ncc4c(N5CC6CCC(C5)N6)nc(OCC5(CN6CCC(N7CCN(c8ccc(NC9CCC(=O)NC9=O)cc8F)CC7)CC6)CC5)nc4c3F)c12] |
| 143 | 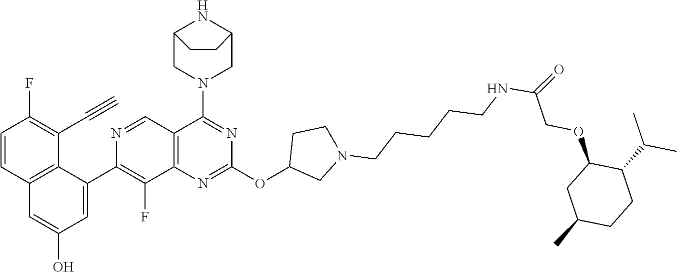 [SMILES: C#Cc1c(F)ccc2cc(O)cc(-c3ncc4c(N5CC6CCC(C5)N6)nc(OC5CCN(CCCCCNC(=O)CO[C@@H]6C[C@H](C)CC[C@H]6C(C)C)C5)nc4c3F)c12] |
| 144 | 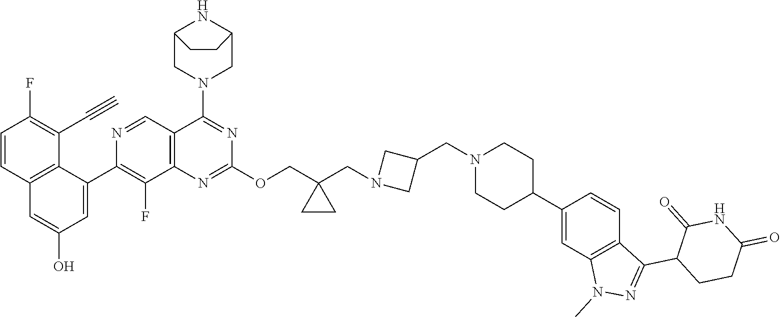 [SMILES: C#Cc1c(F)ccc2cc(O)cc(-c3ncc4c(N5CC6CCC(C5)N6)nc(OCC5(CN6CC(CN7CCC(c8ccc9c(C%10CCC(=O)NC%10=O)nn(C)c9c8)CC7)C6)CC5)nc4c3F)c12] |
| 145 | 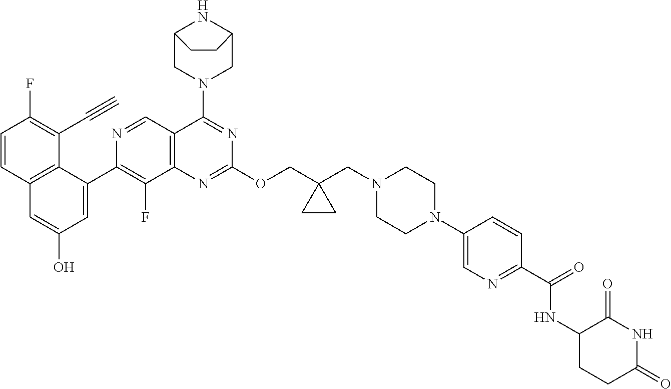 [SMILES: C#Cc1c(F)ccc2cc(O)cc(-c3ncc4c(N5CC6CCC(C5)N6)nc(OCC5(CN6CCN(c7ccc(C(=O)NC8CCC(=O)NC8=O)nc7)CC6)CC5)nc4c3F)c12] |
| 146 | 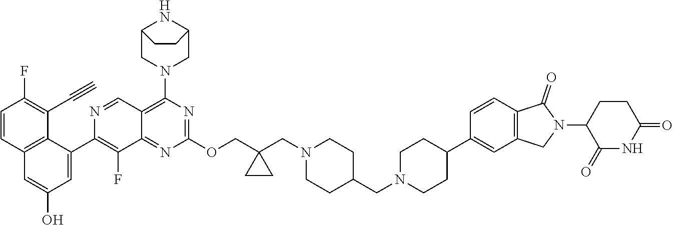 [SMILES: C#Cc1c(F)ccc2cc(O)cc(-c3ncc4c(N5CC6CCC(C5)N6)nc(OCC5(CN6CCC(CN7CCC(c8ccc9c(c8)CN(C8CCC(=O)NC8=O)C9=O)CC7)CC6)CC5)nc4c3F)c12] |
| 147 | 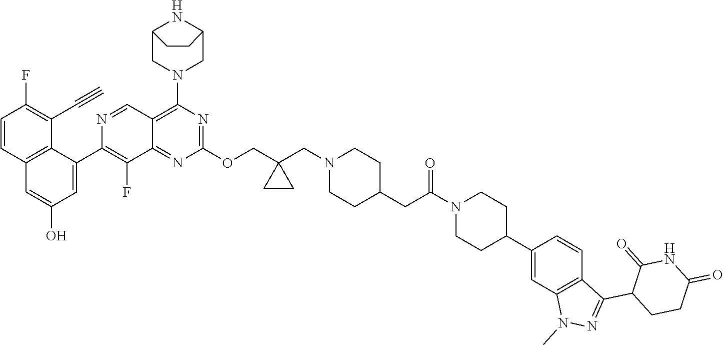 [SMILES: C#Cc1c(F)ccc2cc(O)cc(-c3ncc4c(N5CC6CCC(C5)N6)nc(OCC5(CN6CCC(CC(=O)N7CCC(c8ccc9c(C%10CCC(=O)NC%10=O)nn(C)c9c8)CC7)CC6)CC5)nc4c3F)c12] |
| 148 | 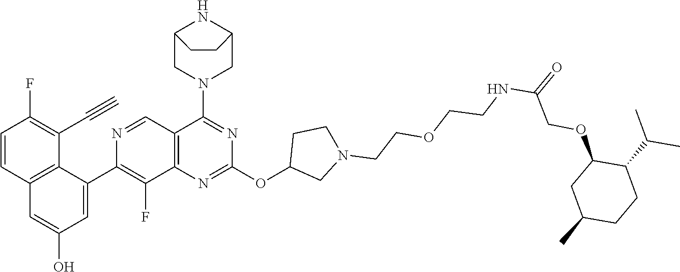 [SMILES: C#Cc1c(F)ccc2cc(O)cc(-c3ncc4c(N5CC6CCC(C5)N6)nc(OC5CCN(CCOCCNC(=O)CO[C@@H]6C[C@H](C)CC[C@H]6C(C)C)C5)nc4c3F)c12] |
| 149 | 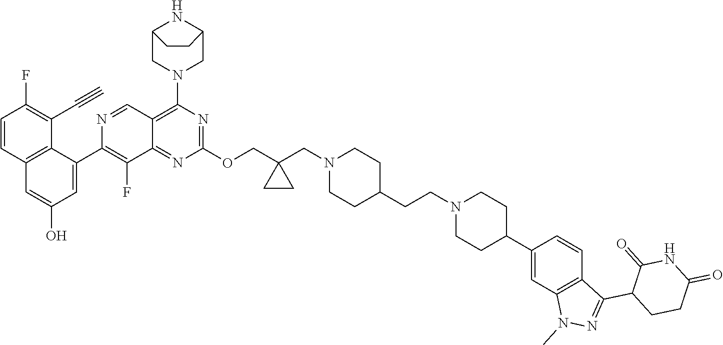 [SMILES: C#Cc1c(F)ccc2cc(O)cc(-c3ncc4c(N5CC6CCC(C5)N6)nc(OCC5(CN6CCC(CCN7CCC(c8ccc9c(C%10CCC(=O)NC%10=O)nn(C)c9c8)CC7)CC6)CC5)nc4c3F)c12] |
| 150 | 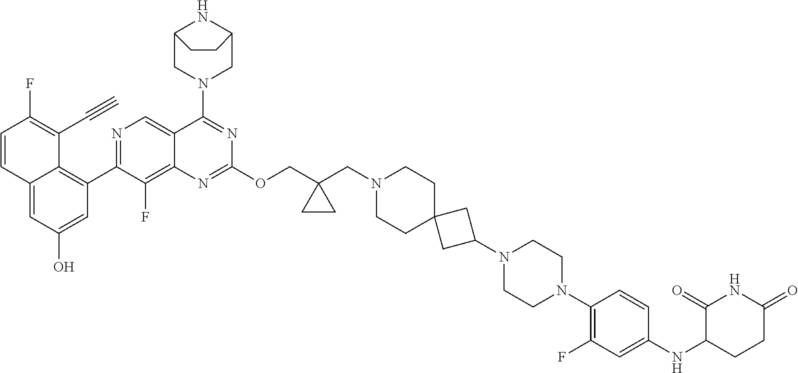 [SMILES: C#Cc1c(F)ccc2cc(O)cc(-c3ncc4c(N5CC6CCC(C5)N6)nc(OCC5(CN6CCC7(CC6)CC(N6CCN(c8ccc(NC9CCC(=O)NC9=O)cc8F)CC6)C7)CC5)nc4c3F)c12] |
| 151 | 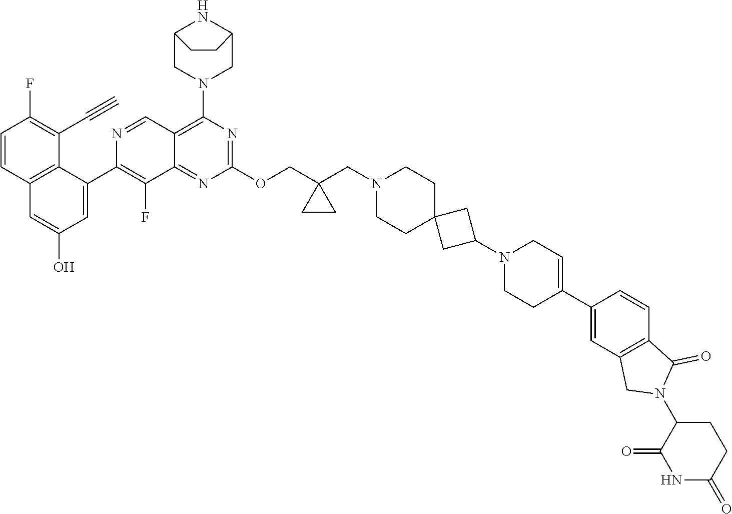 [SMILES: C#Cc1c(F)ccc2cc(O)cc(-c3ncc4c(N5CC6CCC(C5)N6)nc(OCC5(CN6CCC7(CC6)CC(N6CC=C(c8ccc9c(c8)CN(C8CCC(=O)NC8=O)C9=O)CC6)C7)CC5)nc4c3F)c12] |
| 152 | 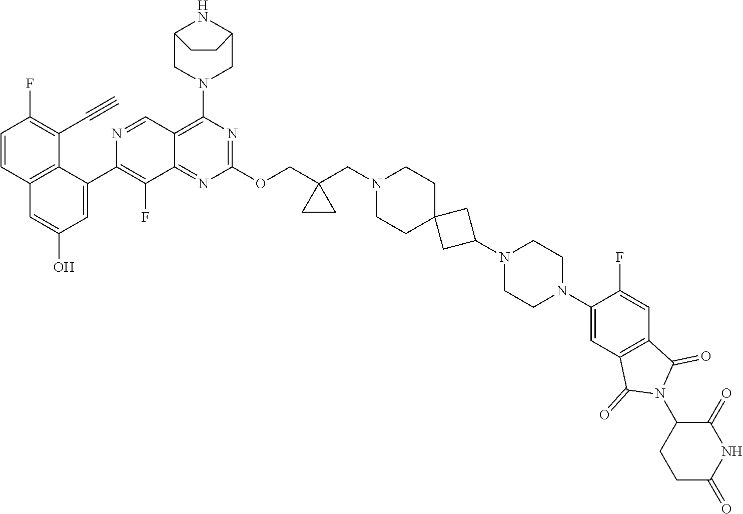 [SMILES: C#Cc1c(F)ccc2cc(O)cc(-c3ncc4c(N5CC6CCC(C5)N6)nc(OCC5(CN6CCC7(CC6)CC(N6CCN(c8cc9c(cc8F)C(=O)N(C8CCC(=O)NC8=O)C9=O)CC6)C7)CC5)nc4c3F)c12] |
| 153 | 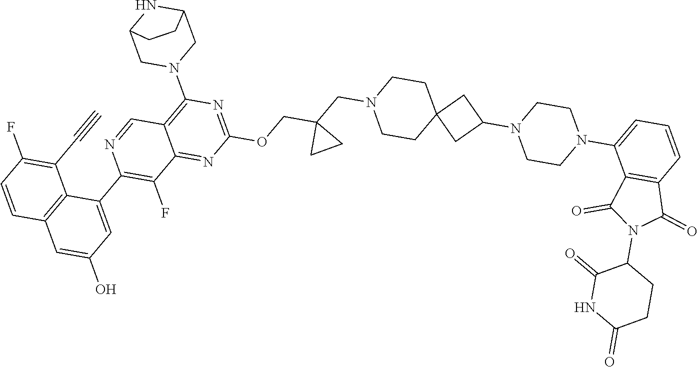 [SMILES: C#Cc1c(F)ccc2cc(O)cc(-c3ncc4c(N5CC6CCC(C5)N6)nc(OCC5(CN6CCC7(CC6)CC(N6CCN(c8cccc9c8C(=O)N(C8CCC(=O)NC8=O)C9=O)CC6)C7)CC5)nc4c3F)c12] |
| 154 | 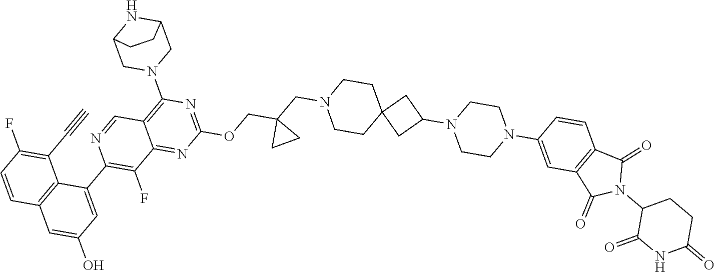 [SMILES: C#Cc1c(F)ccc2cc(O)cc(-c3ncc4c(N5CC6CCC(C5)N6)nc(OCC5(CN6CCC7(CC6)CC(N6CCN(c8ccc9c(c8)C(=O)N(C8CCC(=O)NC8=O)C9=O)CC6)C7)CC5)nc4c3F)c12] |
| 155 | 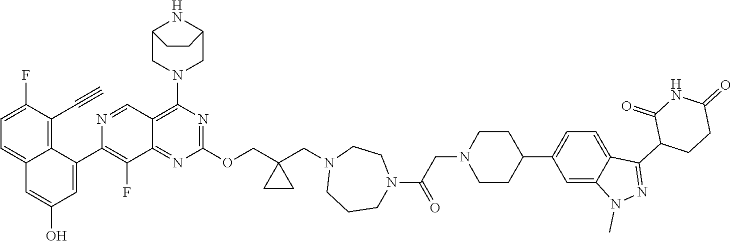 [SMILES: C#Cc1c(F)ccc2cc(O)cc(-c3ncc4c(N5CC6CCC(C5)N6)nc(OCC5(CN6CCCN(C(=O)CN7CCC(c8ccc9c(C%10CCC(=O)NC%10=O)nn(C)c9c8)CC7)CC6)CC5)nc4c3F)c12] |
| 156 | 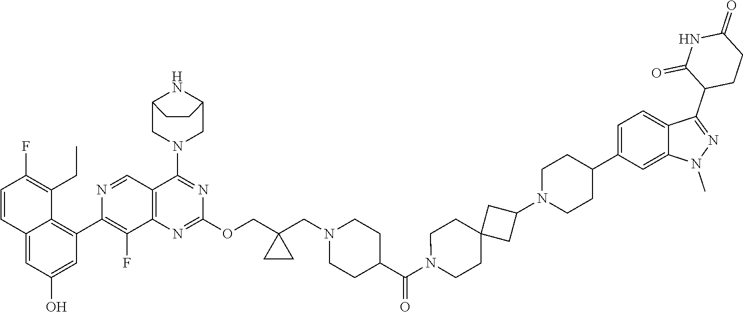 [SMILES: CCc1c(F)ccc2cc(O)cc(-c3ncc4c(N5CC6CCC(C5)N6)nc(OCC5(CN6CCC(C(=O)N7CCC8(CC7)CC(N7CCC(c9ccc%10c(C%11CCC(=O)NC%11=O)nn(C)c%10c9)CC7)C8)CC6)CC5)nc4c3F)c12] |
| 157 | 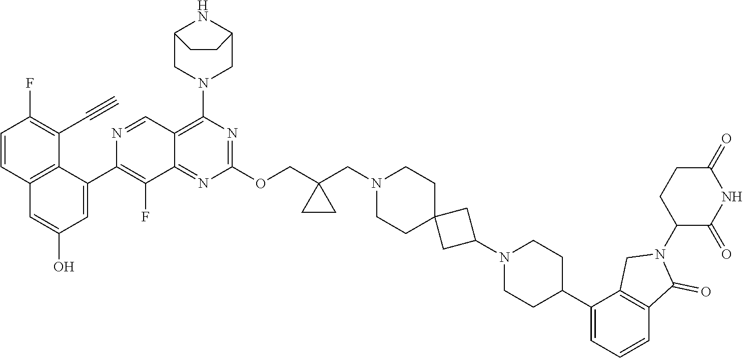 [SMILES: C#Cc1c(F)ccc2cc(O)cc(-c3ncc4c(N5CC6CCC(C5)N6)nc(OCC5(CN6CCC7(CC6)CC(N6CCC(c8cccc9c8CN(C8CCC(=O)NC8=O)C9=O)CC6)C7)CC5)nc4c3F)c12] |
| 158 | 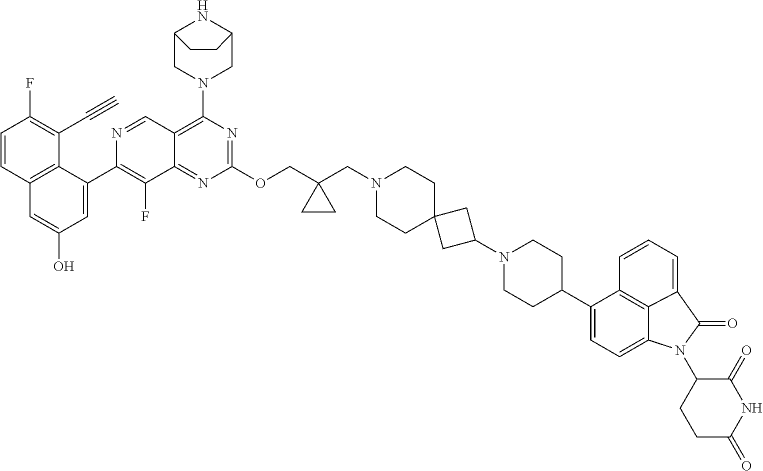 [SMILES: C#Cc1c(F)ccc2cc(O)cc(-c3ncc4c(N5CC6CCC(C5)N6)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12] |
| 159 | 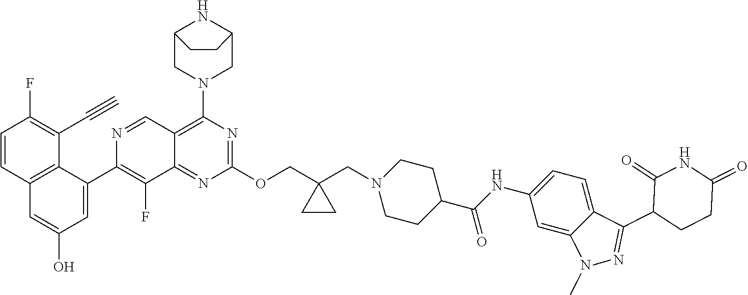 [SMILES: C#Cc1c(F)ccc2cc(O)cc(-c3ncc4c(N5CC6CCC(C5)N6)nc(OCC5(CN6CCC(C(=O)Nc7ccc8c(C9CCC(=O)NC9=O)nn(C)c8c7)CC6)CC5)nc4c3F)c12] |
| 160 | 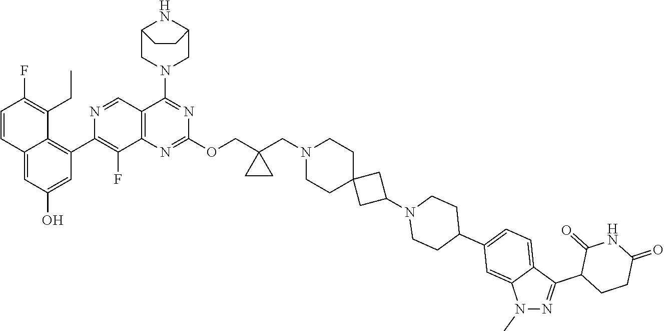 [SMILES: CCc1c(F)ccc2cc(O)cc(-c3ncc4c(N5CC6CCC(C5)N6)nc(OCC5(CN6CCC7(CC6)CC(N6CCC(c8ccc9c(C%10CCC(=O)NC%10=O)nn(C)c9c8)CC6)C7)CC5)nc4c3F)c12] |
| 161 | 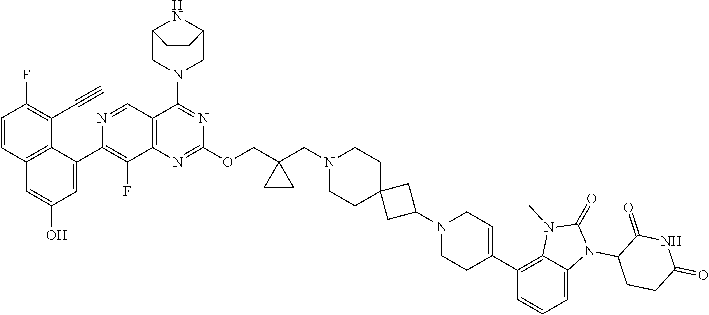 [SMILES: C#Cc1c(F)ccc2cc(O)cc(-c3ncc4c(N5CC6CCC(C5)N6)nc(OCC5(CN6CCC7(CC6)CC(N6CC=C(c8cccc9c8n(C)c(=O)n9C8CCC(=O)NC8=O)CC6)C7)CC5)nc4c3F)c12] |
| 162 | 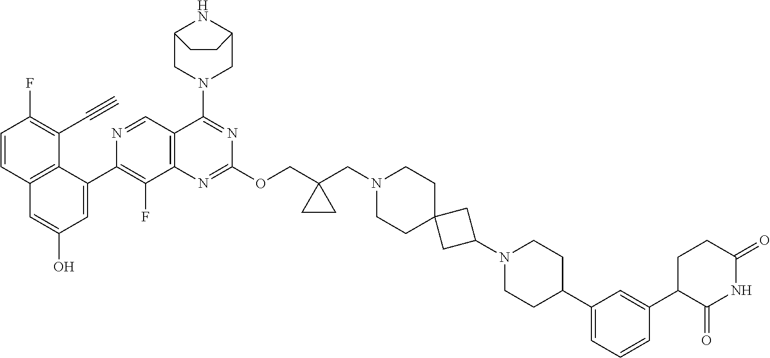 [SMILES: C#Cc1c(F)ccc2cc(O)cc(-c3ncc4c(N5CC6CCC(C5)N6)nc(OCC5(CN6CCC7(CC6)CC(N6CCC(c8cccc(C9CCC(=O)NC9=O)c8)CC6)C7)CC5)nc4c3F)c12] |
| 163 | 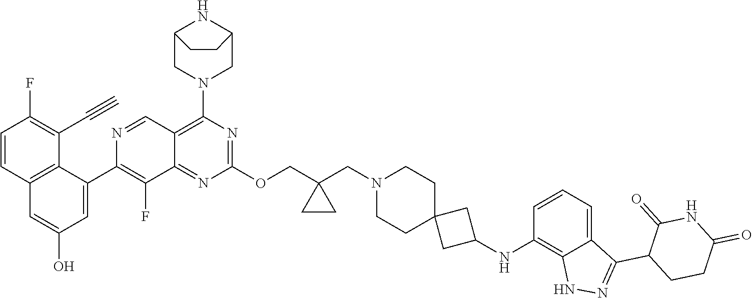 [SMILES: C#Cc1c(F)ccc2cc(O)cc(-c3ncc4c(N5CC6CCC(C5)N6)nc(OCC5(CN6CCC7(CC6)CC(Nc6cccc8c(C9CCC(=O)NC9=O)n[nH]c68)C7)CC5)nc4c3F)c12] |
| 164 | 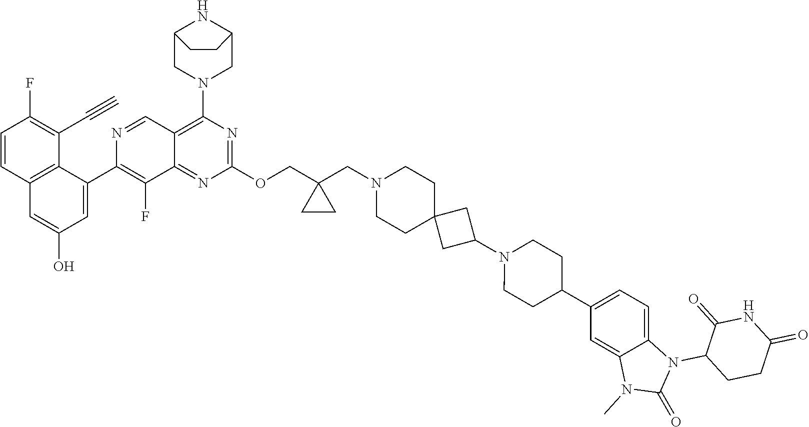 [SMILES: C#Cc1c(F)ccc2cc(O)cc(-c3ncc4c(N5CC6CCC(C5)N6)nc(OCC5(CN6CCC7(CC6)CC(N6CCC(c8ccc9c(c8)n(C)c(=O)n9C8CCC(=O)NC8=O)CC6)C7)CC5)nc4c3F)c12] |
| 165 | 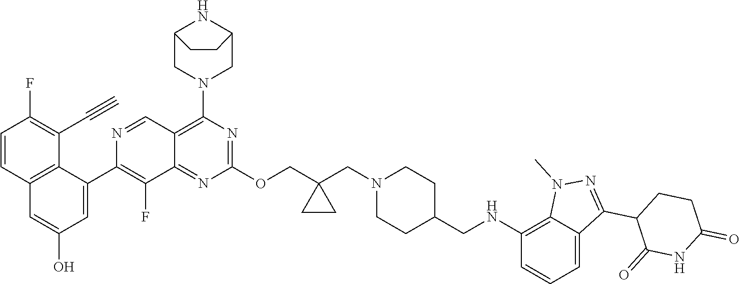 [SMILES: C#Cc1c(F)ccc2cc(O)cc(-c3ncc4c(N5CC6CCC(C5)N6)nc(OCC5(CN6CCC(CNc7cccc8c(C9CCC(=O)NC9=O)nn(C)c78)CC6)CC5)nc4c3F)c12] |
| 166 | 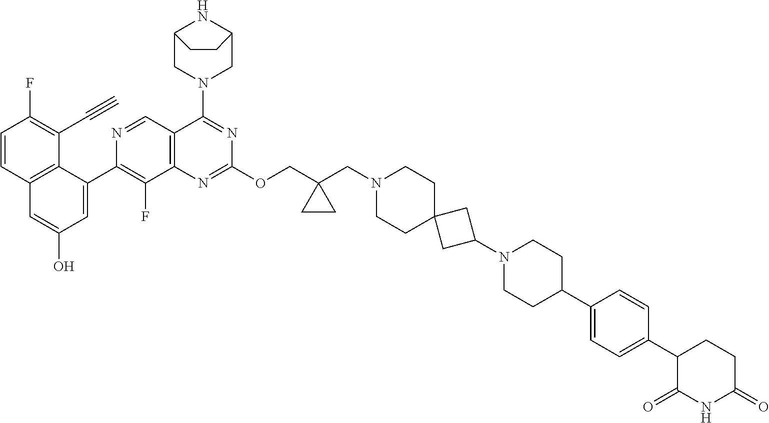 [SMILES: C#Cc1c(F)ccc2cc(O)cc(-c3ncc4c(N5CC6CCC(C5)N6)nc(OCC5(CN6CCC7(CC6)CC(N6CCC(c8ccc(C9CCC(=O)NC9=O)cc8)CC6)C7)CC5)nc4c3F)c12] |
| 167 | 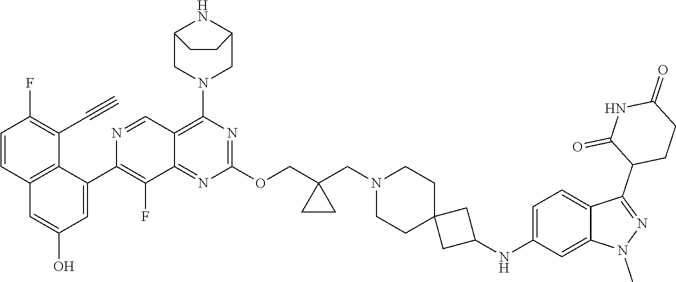 [SMILES: C#Cc1c(F)ccc2cc(O)cc(-c3ncc4c(N5CC6CCC(C5)N6)nc(OCC5(CN6CCC7(CC6)CC(Nc6ccc8c(C9CCC(=O)NC9=O)nn(C)c8c6)C7)CC5)nc4c3F)c12] |
| 168 | 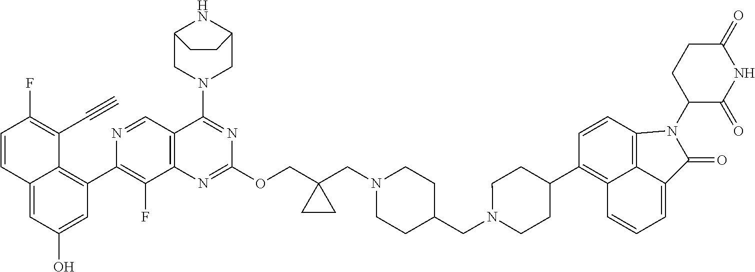 [SMILES: C#Cc1c(F)ccc2cc(O)cc(-c3ncc4c(N5CC6CCC(C5)N6)nc(OCC5(CN6CCC(CN7CCC(c8ccc9c%10c(cccc8%10)C(=O)N9C8CCC(=O)NC8=O)CC7)CC6)CC5)nc4c3F)c12] |
| 169 | 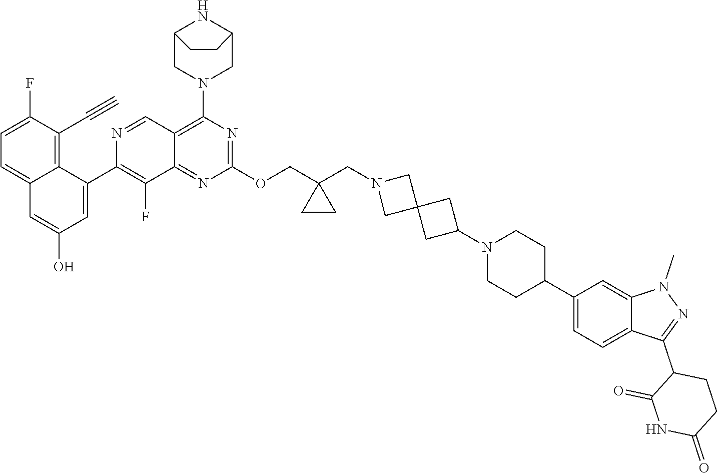 [SMILES: C#Cc1c(F)ccc2cc(O)cc(-c3ncc4c(N5CC6CCC(C5)N6)nc(OCC5(CN6CC7(CC(N8CCC(c9ccc%10c(C%11CCC(=O)NC%11=O)nn(C)c%10c9)CC8)C7)C6)CC5)nc4c3F)c12] |
| 170 | 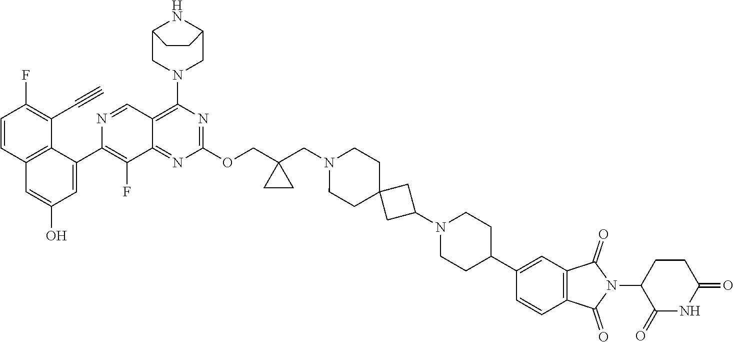 [SMILES: C#Cc1c(F)ccc2cc(O)cc(-c3ncc4c(N5CC6CCC(C5)N6)nc(OCC5(CN6CCC7(CC6)CC(N6CCC(c8ccc9c(c8)C(=O)N(C8CCC(=O)NC8=O)C9=O)CC6)C7)CC5)nc4c3F)c12] |
| 171 | 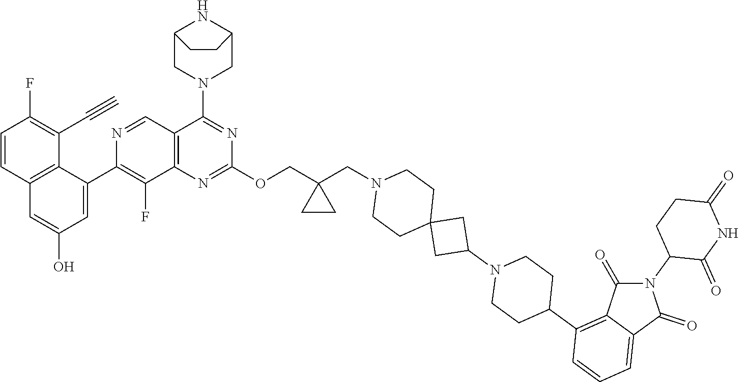 [SMILES: C#Cc1c(F)ccc2cc(O)cc(-c3ncc4c(N5CC6CCC(C5)N6)nc(OCC5(CN6CCC7(CC6)CC(N6CCC(c8cccc9c8C(=O)N(C8CCC(=O)NC8=O)C9=O)CC6)C7)CC5)nc4c3F)c12] |

TABLE 3
Structures of exemplary bifunctional compounds in accordance with certain
embodiments of the disclosure.

| | |
|---|---|
| 1a | 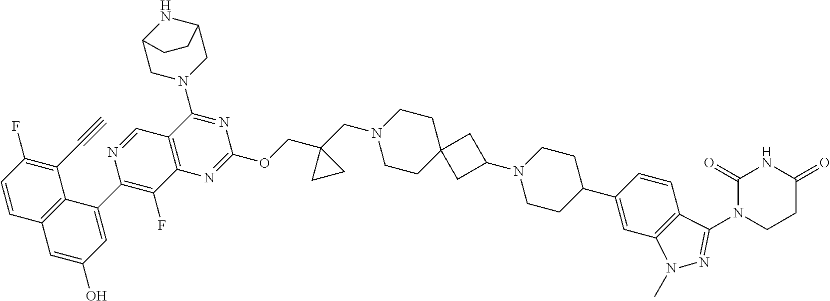 [SMILES: C#Cc1c(F)ccc2cc(O)cc(-c3ncc4c(N5CC6CCC(C5)N6)nc(OCC5(CN6CCC7(CC6)CC(N6CCC(c8ccc9c(N%10CCC(=O)NC%10=O)nn(C)c9c8)CC6)C7)CC5)nc4c3F)c12] |
| 2a | 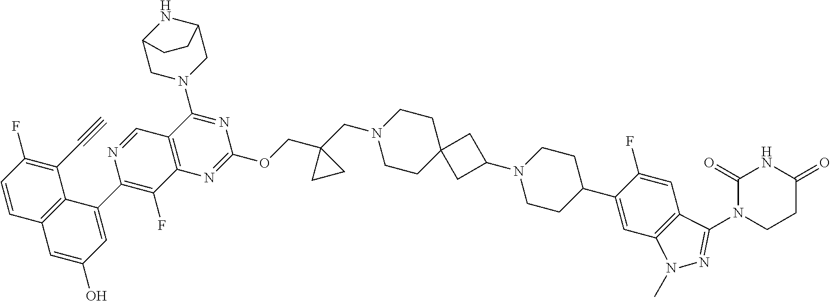 [SMILES: C#Cc1c(F)ccc2cc(O)cc(-c3ncc4c(N5CC6CCC(C5)N6)nc(OCC5(CN6CCC7(CC6)CC(N6CCC(c8cc9c(cc8F)c(N8CCC(=O)NC8=O)nn9C)CC6)C7)CC5)nc4c3F)c12] |
| 3a | 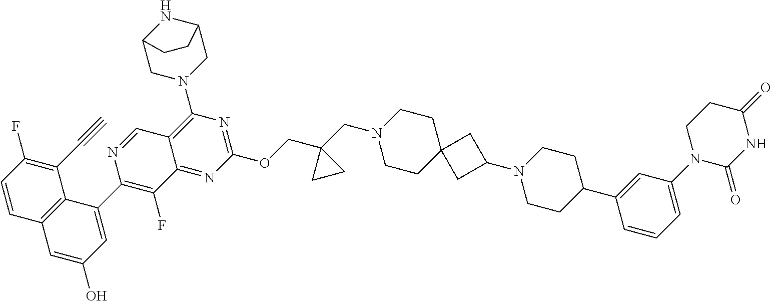 [SMILES: C#Cc1c(F)ccc2cc(O)cc(-c3ncc4c(N5CC6CCC(C5)N6)nc(OCC5(CN6CCC7(CC6)CC(N6CCC(c8cccc(N9CCC(=O)NC9=O)c8)CC6)C7)CC5)nc4c3F)c12] |
| 4a | 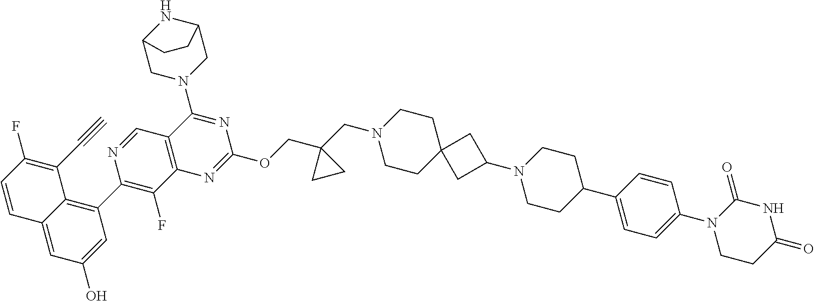 [SMILES: C#Cc1c(F)ccc2cc(O)cc(-c3ncc4c(N5CC6CCC(C5)N6)nc(OCC5(CN6CCC7(CC6)CC(N6CCC(c8ccc(N9CCC(=O)NC9=O)cc8)CC6)C7)CC5)nc4c3F)c12] |
| 5a | 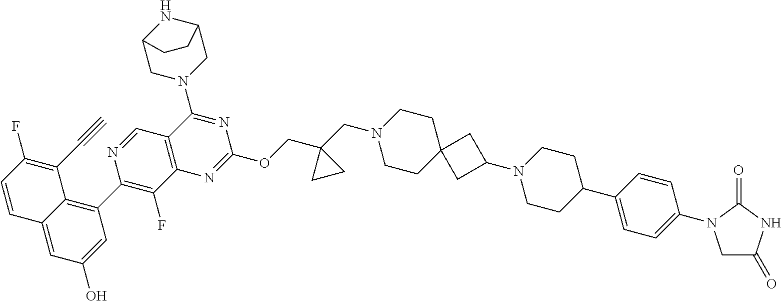 [SMILES: C#Cc1c(F)ccc2cc(O)cc(-c3ncc4c(N5CC6CCC(C5)N6)nc(OCC5(CN6CCC7(CC6)CC(N6CCC(c8ccc(N9CC(=O)NC9=O)cc8)CC6)C7)CC5)nc4c3F)c12] |
| 6a | 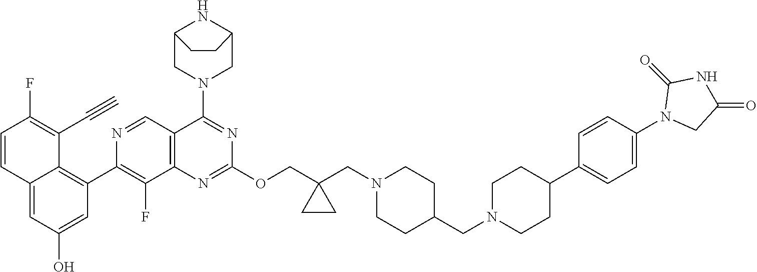 [SMILES: C#Cc1c(F)ccc2cc(O)cc(-c3ncc4c(N5CC6CCC(C5)N6)nc(OCC5(CN6CCC(CN7CCC(c8ccc(N9CC(=O)NC9=O)cc8)CC7)CC6)CC5)nc4c3F)c12] |
| 7a | 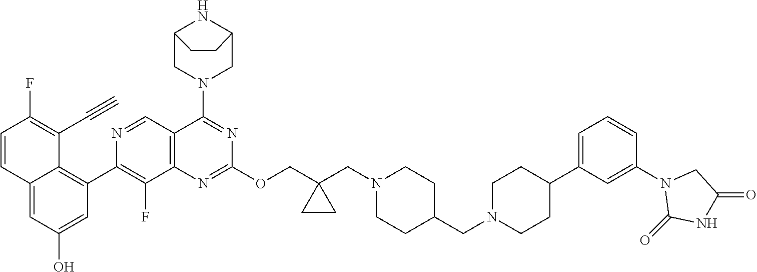 [SMILES: C#Cc1c(F)ccc2cc(O)cc(-c3ncc4c(N5CC6CCC(C5)N6)nc(OCC5(CN6CCC(CN7CCC(c8cccc(N9CC(=O)NC9=O)c8)CC7)CC6)CC5)nc4c3F)c12] |
| 8a | 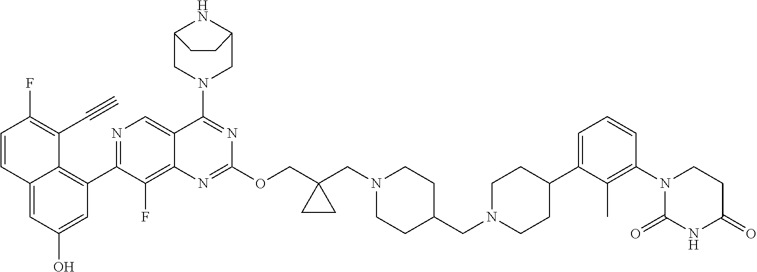 [SMILES: C#Cc1c(F)ccc2cc(O)cc(-c3ncc4c(N5CC6CCC(C5)N6)nc(OCC5(CN6CCC(CN7CCC(c8cccc(N9CCC(=O)NC9=O)c8C)CC7)CC6)CC5)nc4c3F)c12] |
| 9a | 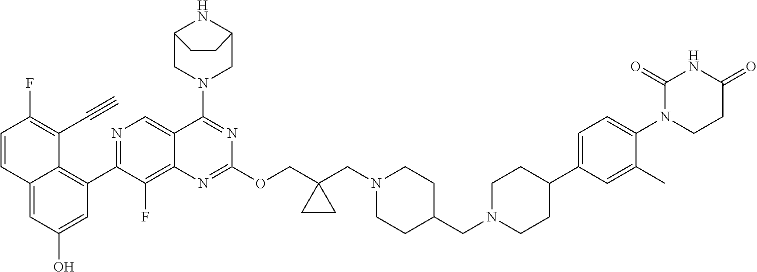 [SMILES: C#Cc1c(F)ccc2cc(O)cc(-c3ncc4c(N5CC6CCC(C5)N6)nc(OCC5(CN6CCC(CN7CCC(c8ccc(N9CCC(=O)NC9=O)c(C)c8)CC7)CC6)CC5)nc4c3F)c12] |
| 10a | 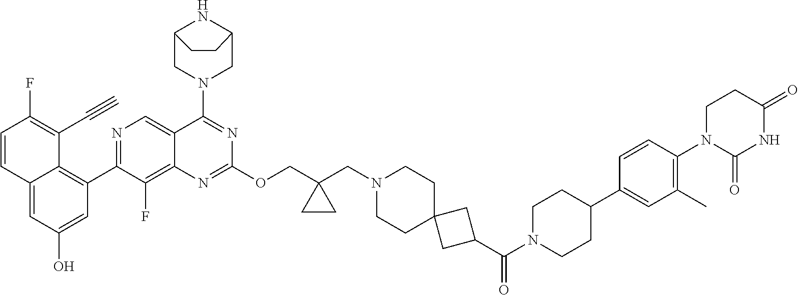 [SMILES: C#Cc1c(F)ccc2cc(O)cc(-c3ncc4c(N5CC6CCC(C5)N6)nc(OCC5(CN6CCC7(CC6)CC(C(=O)N6CCC(c8ccc(N9CCC(=O)NC9=O)c(C)c8)CC6)C7)CC5)nc4c3F)c12] |
| 11a | 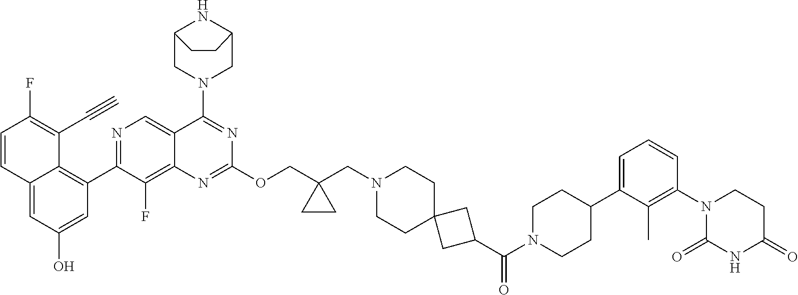 [SMILES: C#Cc1c(F)ccc2cc(O)cc(-c3ncc4c(N5CC6CCC(C5)N6)nc(OCC5(CN6CCC7(CC6)CC(C(=O)N6CCC(c8cccc(N9CCC(=O)NC9=O)c8C)CC6)C7)CC5)nc4c3F)c12] |
| 12a | 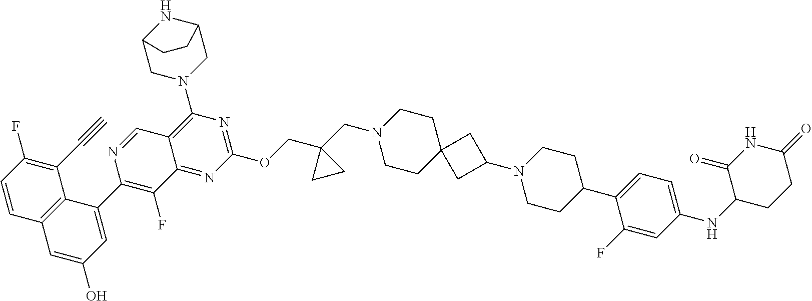 [SMILES: C#Cc1c(F)ccc2cc(O)cc(-c3ncc4c(N5CC6CCC(C5)N6)nc(OCC5(CN6CCC7(CC6)CC(N6CCC(c8ccc(NC9CCC(=O)NC9=O)cc8F)CC6)C7)CC5)nc4c3F)c12] |
| 13a | 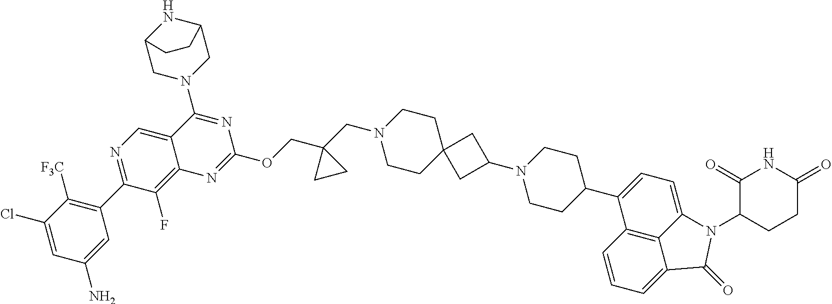 [SMILES: Nc1cc(Cl)c(C(F)(F)F)c(-c2ncc3c(N4CC5CCC(C4)N5)nc(OCC4(CN5CCC6(CC5)CC(N5CCC(c7ccc8c9c(cccc79)C(=O)N8C7CCC(=O)NC7=O)CC5)C6)CC4)nc3c2F)c1] |
| 14a | 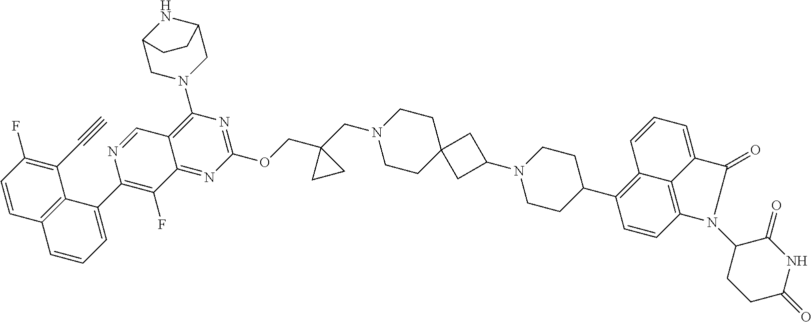 [SMILES: C#Cc1c(F)ccc2cccc(-c3ncc4c(N5CC6CCC(C5)N6)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12] |
| 15a | 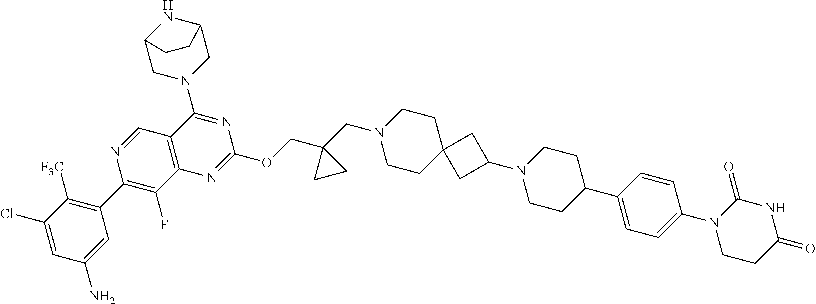 [SMILES: Nc1cc(Cl)c(C(F)(F)F)c(-c2ncc3c(N4CC5CCC(C4)N5)nc(OCC4(CN5CCC6(CC5)CC(N5CCC(c7ccc(N8CCC(=O)NC8=O)cc7)CC5)C6)CC4)nc3c2F)c1] |
| 16a | 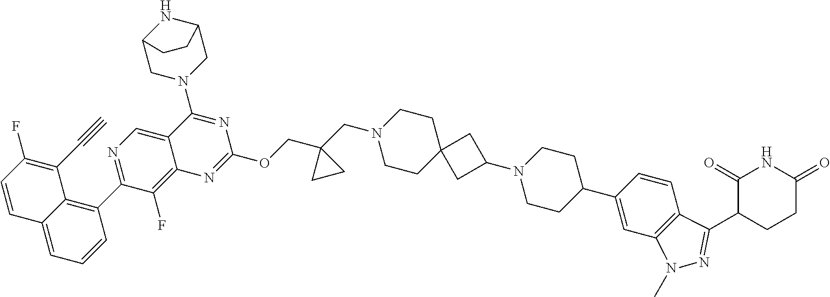 [SMILES: C#Cc1c(F)ccc2cccc(-c3ncc4c(N5CC6CCC(C5)N6)nc(OCC5(CN6CCC7(CC6)CC(N6CCC(c8ccc9c(C%10CCC(=O)NC%10=O)nn(C)c9c8)CC6)C7)CC5)nc4c3F)c12] |
| 17a | 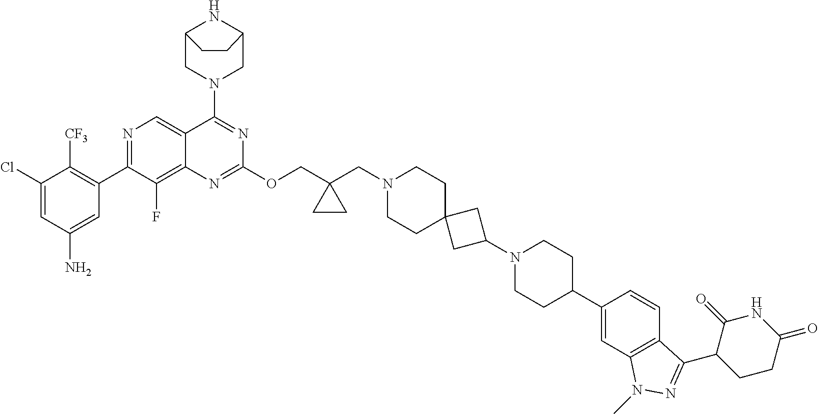 [SMILES: Cn1nc(C2CCC(=O)NC2=O)c2ccc(C3CCN(C4CC5(CCN(CC6(COc7nc(N8CC9CCC(C8)N9)c8cnc(-c9cc(N)cc(Cl)c9C(F)(F)F)c(F)c8n7)CC6)CC5)C4)CC3)cc21] |
| 18a | 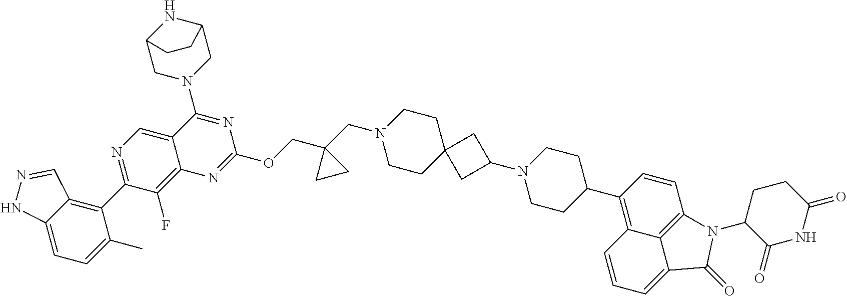 [SMILES: Cc1ccc2[nH]ncc2c1-c1ncc2c(N3CC4CCC(C3)N4)nc(OCC3(CN4CCC5(CC4)CC(N4CCC(c6ccc7c8c(cccc68)C(=O)N7C6CCC(=O)NC6=O)CC4)C5)CC3)nc2c1F] |
| 19a | 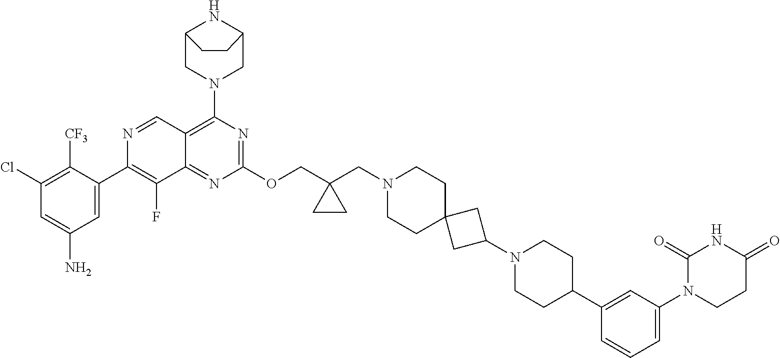 [SMILES: Nc1cc(Cl)c(C(F)(F)F)c(-c2ncc3c(N4CC5CCC(C4)N5)nc(OCC4(CN5CCC6(CC5)CC(N5CCC(c7cccc(N8CCC(=O)NC8=O)c7)CC5)C6)CC4)nc3c2F)c1] |
| 20a | 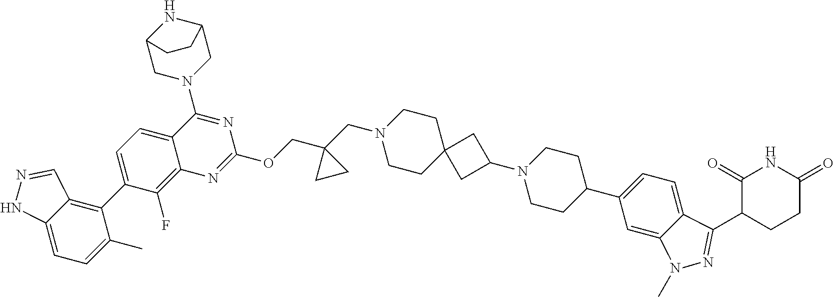 [SMILES: Cc1ccc2[nH]ncc2c1-c1ccc2c(N3CC4CCC(C3)N4)nc(OCC3(CN4CCC5(CC4)CC(N4CCC(c6ccc7c(C8CCC(=O)NC8=O)nn(C)c7c6)CC4)C5)CC3)nc2c1F] |
| 21a | 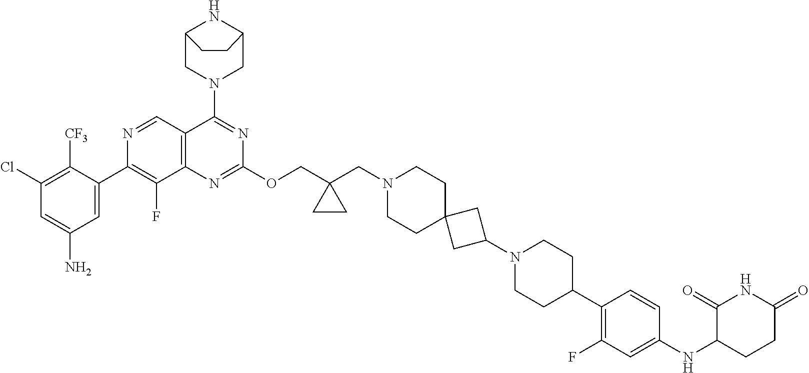 [SMILES: Nc1cc(Cl)c(C(F)(F)F)c(-c2ncc3c(N4CC5CCC(C4)N5)nc(OCC4(CN5CCC6(CC5)CC(N5CCC(c7ccc(NC8CCC(=O)NC8=O)cc7F)CC5)C6)CC4)nc3c2F)c1] |
| 22a | 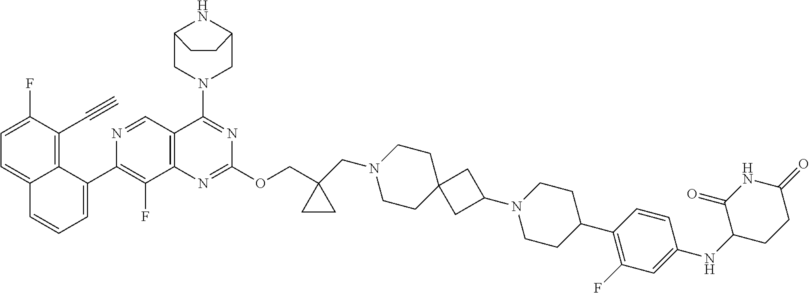 [SMILES: C#Cc1c(F)ccc2cccc(-c3ncc4c(N5CC6CCC(C5)N6)nc(OCC5(CN6CCC7(CC6)CC(N6CCC(c8ccc(NC9CCC(=O)NC9=O)cc8F)CC6)C7)CC5)nc4c3F)c12] |
| 23a | 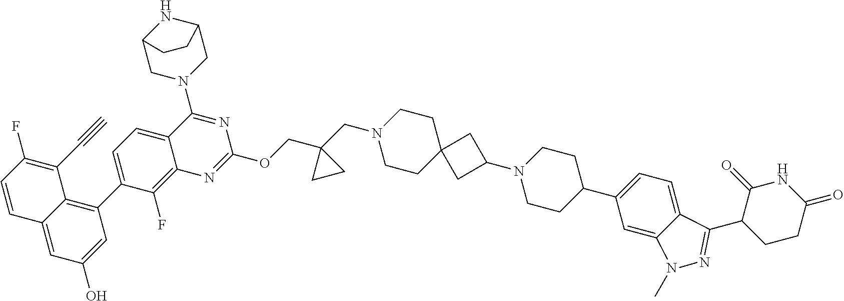 [SMILES: C#Cc1c(F)ccc2cc(O)cc(-c3ccc4c(N5CC6CCC(C5)N6)nc(OCC5(CN6CCC7(CC6)CC(N6CCC(c8ccc9c(C%10CCC(=O)NC%10=O)nn(C)c9c8)CC6)C7)CC5)nc4c3F)c12] |
| 24a | 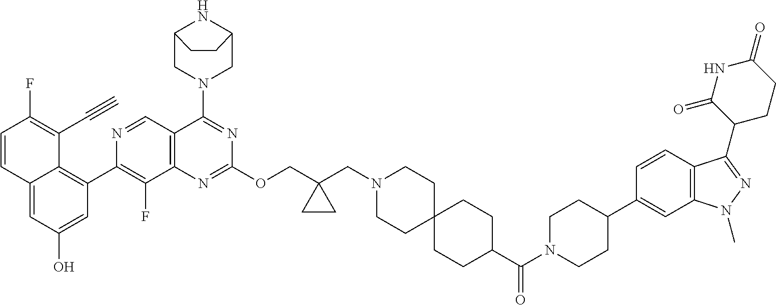 [SMILES: C#Cc1c(F)ccc2cc(O)cc(-c3ncc4c(N5CC6CCC(C5)N6)nc(OCC5(CN6CCC7(CCC(C(=O)N8CCC(c9ccc%10c(C%11CCC(=O)NC%11=O)nn(C)c%10c9)CC8)CC7)CC6)CC5)nc4c3F)c12] |
| 25a | 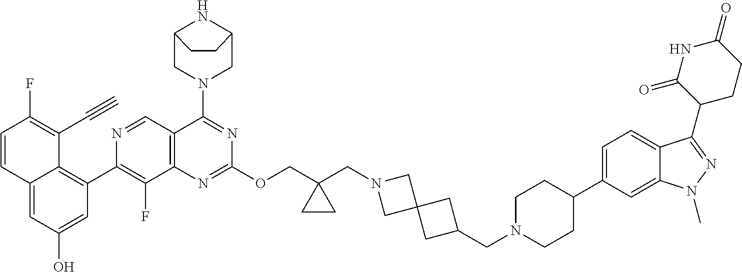 [SMILES: C#Cc1c(F)ccc2cc(O)cc(-c3ncc4c(N5CC6CCC(C5)N6)nc(OCC5(CN6CC7(CC(CN8CCC(c9ccc%10c(C%11CCC(=O)NC%11=O)nn(C)c%10c9)CC8)C7)C6)CC5)nc4c3F)c12] |
| 26a | 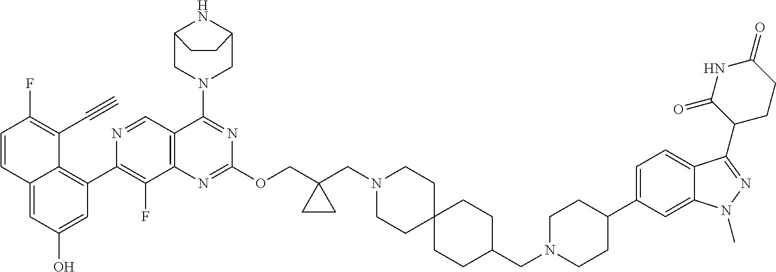 [SMILES: C#Cc1c(F)ccc2cc(O)cc(-c3ncc4c(N5CC6CCC(C5)N6)nc(OCC5(CN6CCC7(CCC(CN8CCC(c9ccc%10c(C%11CCC(=O)NC%11=O)nn(C)c%10c9)CC8)CC7)CC6)CC5)nc4c3F)c12] |
| 27a | 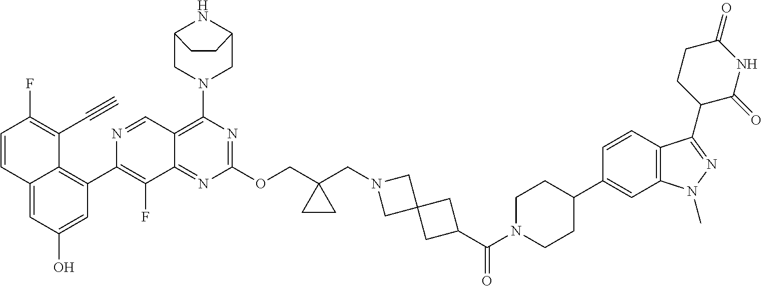 [SMILES: C#Cc1c(F)ccc2cc(O)cc(-c3ncc4c(N5CC6CCC(C5)N6)nc(OCC5(CN6CC7(CC(C(=O)N8CCC(c9ccc%10c(C%11CCC(=O)NC%11=O)nn(C)c%10c9)CC8)C7)C6)CC5)nc4c3F)c12] |
| 28a | 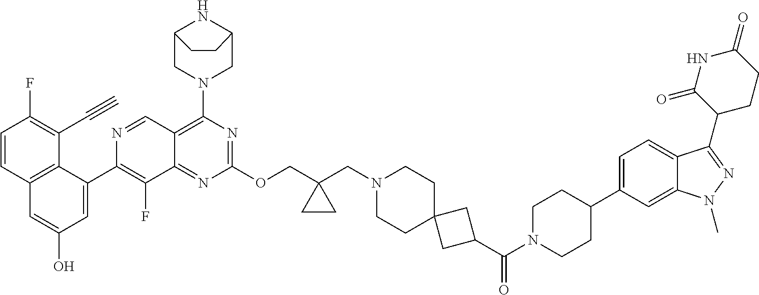 [SMILES: C#Cc1c(F)ccc2cc(O)cc(-c3ncc4c(N5CC6CCC(C5)N6)nc(OCC5(CN6CCC7(CC6)CC(C(=O)N6CCC(c8ccc9c(C%10CCC(=O)NC%10=O)nn(C)c9c8)CC6)C7)CC5)nc4c3F)c12] |
| 29a | 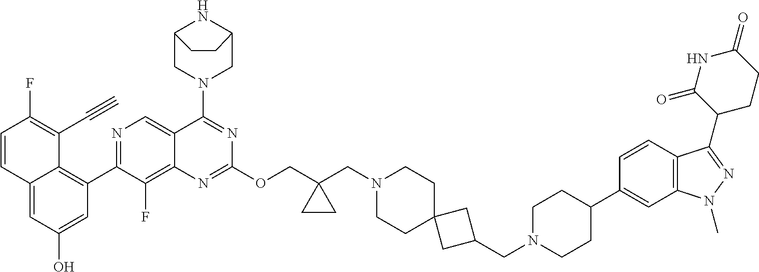 [SMILES: C#Cc1c(F)ccc2cc(O)cc(-c3ncc4c(N5CC6CCC(C5)N6)nc(OCC5(CN6CCC7(CC6)CC(CN6CCC(c8ccc9c(C%10CCC(=O)NC%10=O)nn(C)c9c8)CC6)C7)CC5)nc4c3F)c12] |
| 30a | 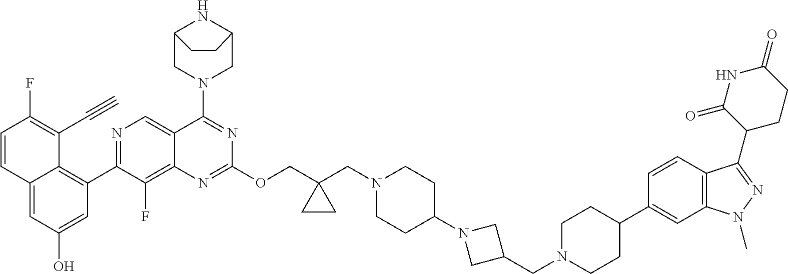 [SMILES: C#Cc1c(F)ccc2cc(O)cc(-c3ncc4c(N5CC6CCC(C5)N6)nc(OCC5(CN6CCC(N7CC(CN8CCC(c9ccc%10c(C%11CCC(=O)NC%11=O)nn(C)c%10c9)CC8)C7)CC6)CC5)nc4c3F)c12] |
| 31a | 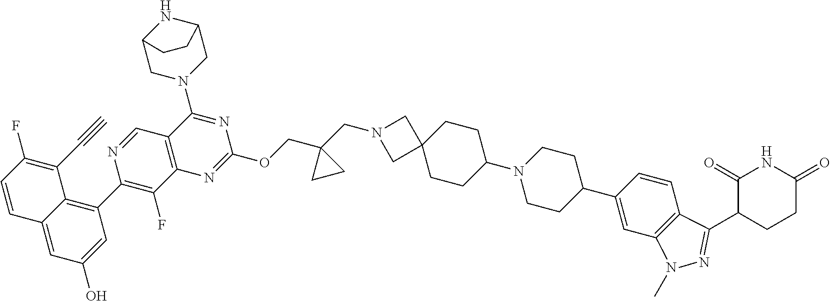 [SMILES: C#Cc1c(F)ccc2cc(O)cc(-c3ncc4c(N5CC6CCC(C5)N6)nc(OCC5(CN6CC7(CCC(N8CCC(c9ccc%10c(C%11CCC(=O)NC%11=O)nn(C)c%10c9)CC8)CC7)C6)CC5)nc4c3F)c12] |
| 32a | 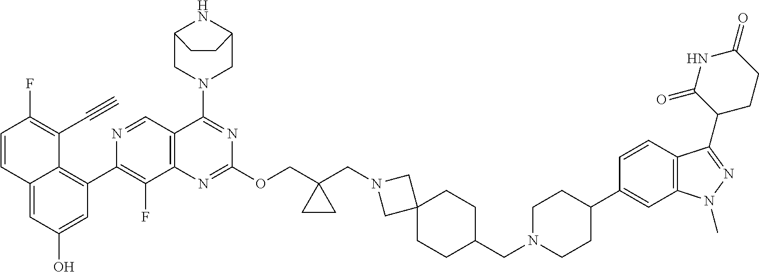 [SMILES: C#Cc1c(F)ccc2cc(O)cc(-c3ncc4c(N5CC6CCC(C5)N6)nc(OCC5(CN6CC7(CCC(CN8CCC(c9ccc%10c(C%11CCC(=O)NC%11=O)nn(C)c%10c9)CC8)CC7)C6)CC5)nc4c3F)c12] |
| 33a | 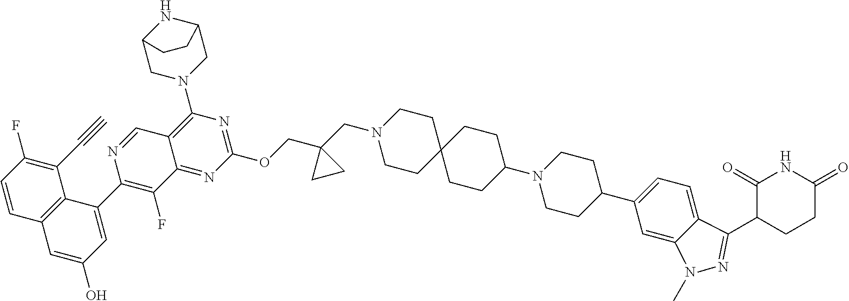 [SMILES: C#Cc1c(F)ccc2cc(O)cc(-c3ncc4c(N5CC6CCC(C5)N6)nc(OCC5(CN6CCC7(CCC(N8CCC(c9ccc%10c(C%11CCC(=O)NC%11=O)nn(C)c%10c9)CC8)CC7)CC6)CC5)nc4c3F)c12] |
| 34a | 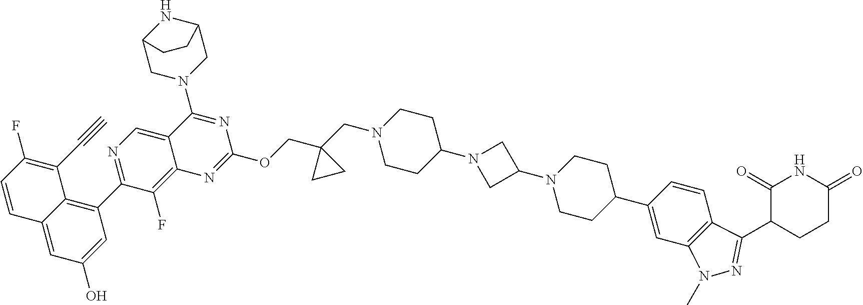 [SMILES: C#Cc1c(F)ccc2cc(O)cc(-c3ncc4c(N5CC6CCC(C5)N6)nc(OCC5(CN6CCC(N7CC(N8CCC(c9ccc%10c(C%11CCC(=O)NC%11=O)nn(C)c%10c9)CC8)C7)CC6)CC5)nc4c3F)c12] |
| 35a | 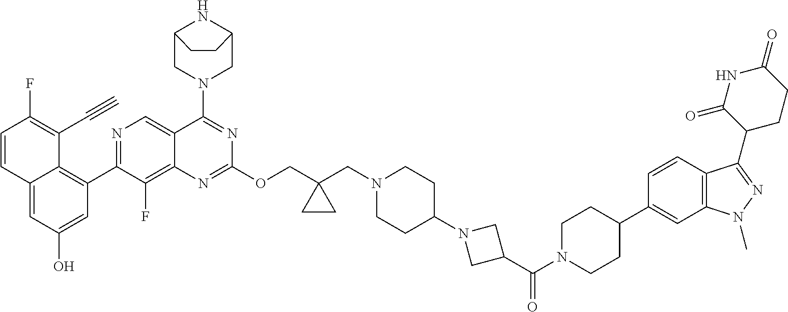 [SMILES: C#Cc1c(F)ccc2cc(O)cc(-c3ncc4c(N5CC6CCC(C5)N6)nc(OCC5(CN6CCC(N7CC(C(=O)N8CCC(c9ccc%10c(C%11CCC(=O)NC%11=O)nn(C)c%10c9)CC8)C7)CC6)CC5)nc4c3F)c12] |
| 36aa | 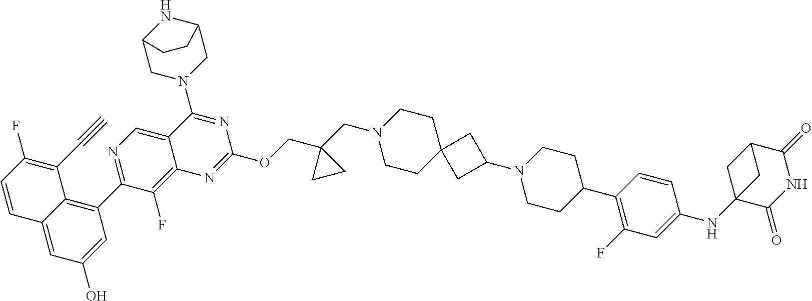 [SMILES: C#Cc1c(F)ccc2cc(O)cc(-c3ncc4c(N5CC6CCC(C5)N6)nc(OCC5(CN6CCC7(CC6)CC(N6CCC(c8ccc(NC9%10CC(C9)C(=O)NC%10=O)cc8F)CC6)C7)CC5)nc4c3F)c12] |
| 37a | 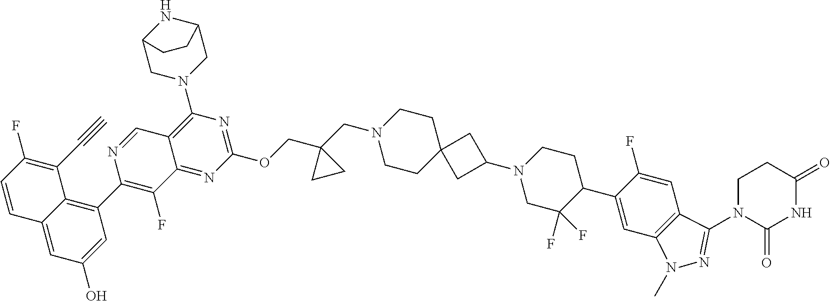 [SMILES: C#Cc1c(F)ccc2cc(O)cc(-c3ncc4c(N5CC6CCC(C5)N6)nc(OCC5(CN6CCC7(CC6)CC(N6CCC(c8cc9c(cc8F)c(N8CCC(=O)NC8=O)nn9C)C(F)(F)C6)C7)CC5)nc4c3F)c12] |
| 38a | 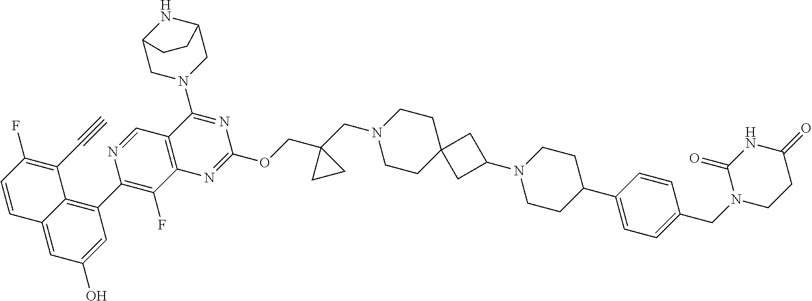 [SMILES: C#Cc1c(F)ccc2cc(O)cc(-c3ncc4c(N5CC6CCC(C5)N6)nc(OCC5(CN6CCC7(CC6)CC(N6CCC(c8ccc(CN9CCC(=O)NC9=O)cc8)CC6)C7)CC5)nc4c3F)c12] |
| 39a | 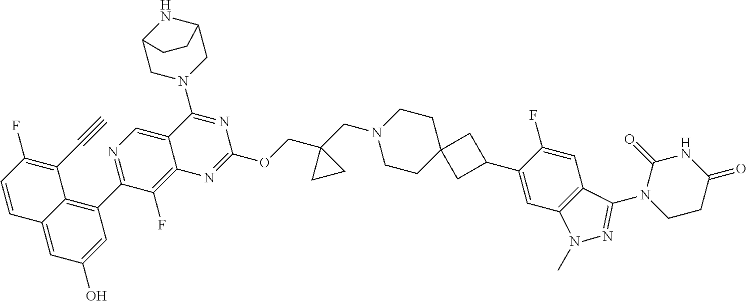 [SMILES: C#Cc1c(F)ccc2cc(O)cc(-c3ncc4c(N5CC6CCC(C5)N6)nc(OCC5(CN6CCC7(CC6)CC(c6cc8c(cc6F)c(N6CCC(=O)NC6=O)nn8C)C7)CC5)nc4c3F)c12] |
| 40a | 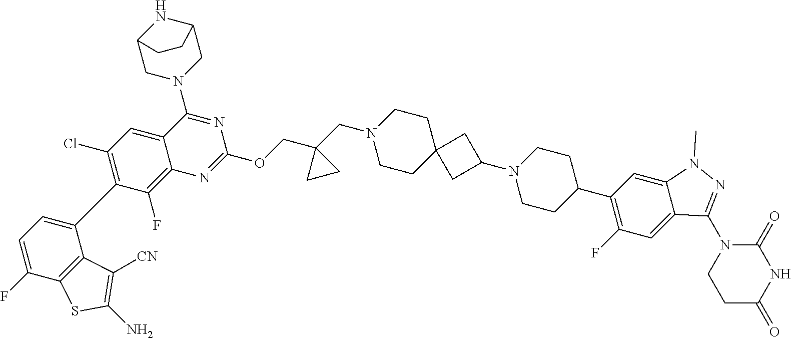 [SMILES: Cn1nc(N2CCC(=O)NC2=O)c2cc(F)c(C3CCN(C4CC5(CCN(CC6(COc7nc(N8CC9CCC(C8)N9)c8cc(Cl)c(-c9ccc(F)c%10sc(N)c(C#N)c9%10)c(F)c8n7)CC6)CC5)C4)CC3)cc21] |
| 41a | 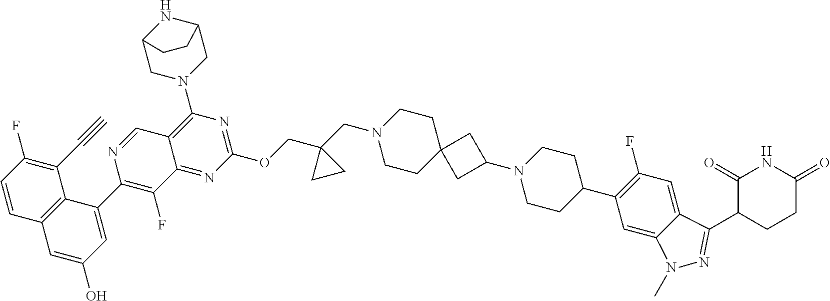 [SMILES: C#Cc1c(F)ccc2cc(O)cc(-c3ncc4c(N5CC6CCC(C5)N6)nc(OCC5(CN6CCC7(CC6)CC(N6CCC(c8cc9c(cc8F)c(C8CCC(=O)NC8=O)nn9C)CC6)C7)CC5)nc4c3F)c12] |

In some embodiments, there is provided a compound of Table 1, or a pharmaceutically acceptable salt, ester, stereoisomer, hydrate, or solvate thereof.

In some embodiments, there is provided a compound of Table 2, or a pharmaceutically acceptable salt, ester, stereoisomer, hydrate, or solvate thereof.

In some embodiments, there is provided a compound of Table 3, or a pharmaceutically acceptable salt, ester, stereoisomer, hydrate, or solvate thereof.

For compounds of the disclosure, when a chiral center is present, it should be understood that the configuration of the stereoisomer is not limited. Thus, when a chiral center is present, the configuration of the stereoisomer may be R-configuration, S-configuration, or a mixture of R- and S-configurations. All isomeric forms, including stereoisomers, diastereoisomers, and the like are intended to be included.

In some embodiments, there is provided a compound as described herein wherein the C, H, O, and N atoms in the compound are each independently selected from atoms of natural abundance and isotope-enriched atoms. Examples of isotopes of natural abundance include ¹²C, ¹H, ¹⁶O and ¹⁴N. Examples of isotope-enriched atoms include, without limitation, ¹³C and ¹⁴C for carbon; ²H (D) and ³H (T) for hydrogen; ¹⁷O and ¹⁸O for oxygen; and ¹⁵N for nitrogen. In some embodiments of compounds of the disclosure, all the elements or atoms in a compound are isotopes of natural abundance. In other embodiments, one or more elements or atoms in a compound are isotope-enriched.

In another broad aspect, there are provided pharmaceutical compositions comprising a compound described herein, or a pharmaceutically acceptable salt, ester, hydrate, solvate or stereoisomer thereof, and a pharmaceutically acceptable excipient, carrier or diluent. In some embodiments, there are provided pharmaceutical compositions comprising a compound of Formula (I), or a pharmaceutically acceptable salt, ester, hydrate, solvate or stereoisomer thereof, and a pharmaceutically acceptable carrier. In some embodiments, there are provided pharmaceutical compositions comprising a compound of Table 1, or a pharmaceutically acceptable salt, ester, hydrate, solvate or stereoisomer thereof, and a pharmaceutically acceptable carrier. In some embodiments, there are provided pharmaceutical compositions comprising a compound of Table 2, or a pharmaceutically acceptable salt, ester, hydrate, solvate or stereoisomer thereof, and a pharmaceutically acceptable carrier. In some embodiments, there are provided pharmaceutical compositions comprising a compound of Table 3, or a pharmaceutically acceptable salt, ester, hydrate, solvate or stereoisomer thereof, and a pharmaceutically acceptable carrier.

In some such embodiments, the composition comprises a pharmaceutically acceptable excipient comprising one or more adhesive, filler, disintegrant, lubricant, and/or dispersant. In some embodiments, the pharmaceutically acceptable carrier comprises a cream, an emulsion, a gel, a liposome, or a nanoparticle.

In some embodiments, the pharmaceutical composition is suitable for oral administration. In some such embodiments, the composition is in the form of a hard shell gelatin capsule, a soft shell gelatin capsule, a cachet, a pill, a tablet, a lozenge, a powder, a granule, a pellet, a pastille, or a dragee. In some embodiments, the composition is in the form of a solution, an aqueous liquid suspension, a non-aqueous liquid suspension, an oil-in-water liquid emulsion, a water-in-oil liquid emulsion, an elixir, or a syrup. In some embodiments, the composition is enteric coated. In some embodiments, the composition is formulated for controlled release.

In some embodiments, the pharmaceutical composition is injectable.

In some embodiments, the pharmaceutically acceptable carrier further comprises at least one additional therapeutic agent, such as, without limitation, a chemotherapeutic agent or another anti-cancer agent. In an embodiment, the at least one additional therapeutic agent is an immune checkpoint inhibitor. Non-limiting examples of immune checkpoint inhibitors include ipilimumab, nivolumab and lambrolizumab.

In another broad aspect, there are provided methods of inhibiting KRAS-G12D activity in a subject in need thereof, comprising administering to the subject an effective amount of a compound and/or a pharmaceutical composition described herein.

In certain embodiments, there are provided methods of treating or preventing a KRAS-G12D-associated disease, disorder or condition in a subject in need thereof, comprising administering an effective amount of a compound and/or a pharmaceutical composition described herein, such that the KRAS-G12D-associated disease, disorder or condition is treated or prevented in the subject.

In particular embodiments, the compounds described herein act to inhibit KRAS-G12D and are useful as therapeutic or prophylactic therapy when such inhibition is desired, e.g., for the prevention or treatment of KRAS-G12D-associated diseases, conditions and/or disorders. Unless otherwise indicated, when uses of the compounds of the present disclosure are described herein, it is to be understood that such compounds may be in the form of a composition (e.g., a pharmaceutical composition). As used herein, the terms “KRAS-G12D inhibitor” and “bifunctional compound” are used interchangeably to refer to a compound of the disclosure capable of inhibiting and/or degrading the KRAS-G12D protein in a cellular assay, an in vivo model, and/or other assay means indicative of KRAS-G12D inhibition and potential therapeutic or prophylactic efficacy. “KRAS-G12D inhibition” includes inter alia modulation or promotion of degradation of the KRAS-G12D protein, e.g., via a Protacs-type mechanism. The terms also refer to compounds that exhibit at least some therapeutic or prophylactic benefit in a human subject. Although the compounds of the present invention are believed to have effect by promoting degradation of KRAS-G12D in a cell, a precise understanding of the compounds' underlying mechanism of action is not required to practice the invention.

In some embodiments, there are provided methods for preventing or treating a KRAS-G12D-associated disease, disorder or condition in a subject in need thereof. The KRAS-G12D-associated disease, disorder or condition may be, for example and without limitation, a cancer or tumor or hyperplastic or hyperproliferative disease or disorder related to or associated with the KRAS-G12D mutation. In some embodiments, the KRAS-G12D-associated disease, disorder or condition is a hyperplastic disorder. In some embodiments, the KRAS-G12D-associated disease, disorder or condition is a malignant cancer or tumor. In some embodiments, the KRAS-G12D-associated disease, disorder or condition is a cardiac, lung, gastrointestinal, genitourinary tract, liver, bone, nervous system, gynecological, hematologic, skin, or adrenal gland cancer or tumor. In some embodiments, the KRAS-G12D-associated disease, disorder or condition is a non-small-cell lung cancer (NSCLC), a small cell lung cancer, a pancreatic cancer, a colorectal cancer, a colon cancer, a bile duct cancer, a cervical cancer, a bladder cancer, a liver cancer or a breast cancer.

In some embodiments, there are provided methods for treating or preventing cancer in a subject (e.g., a human) comprising administering to the subject a therapeutically effective amount of at least one KRAS-G12D inhibitor compound or composition described herein. In some embodiments of such methods, the subject is administered at least one KRAS-G12D inhibitor compound or composition in an amount effective to reverse, slow or stop the progression of a KRAS-G12D-associated disease, disorder or condition.

The type of cancer or tumor that can be treated or prevented using the compounds and compositions described herein is not meant to be particularly limited. Examples of cancers and tumors that can be treated or prevented using the compounds and compositions described herein include, but are not limited to, cancers of the: (i) cardiac tissue or heart (including sarcoma, angiosarcoma, fibrosarcoma, rhabdomyosarcoma, liposarcoma, myxoma, rhabdomyoma, fibroma, lipoma, teratoma); (ii) lung (including bronchogenic carcinoma, squamous cell carcinoma, undifferentiated small cell carcinoma, undifferentiated large cell carcinoma, adenocarcinoma, alveolar (bronchiolar) carcinoma, bronchial adenoma, sarcoma, lymphoma, chondromatous hamartoma, mesothelioma); (iii) gastrointestinal system (including esophagus (squamous, cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, Karposi's sarcoma, leiomyoma, hemangioma, lipoma, neurofibroma, fibroma), large bowel (adenocarcinomas, tubular adenoma, villous adenoma, hamartoma, leiomyoma)); (iv) genitourinary tract (including kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embroyonal carcinoma, teratocarcinoma, choriocarcinoma, sarcoma, interstitial cell carcinoma, fibroma, fibroadenoma, adenomatoid rumors, lipoma); (v) liver (including hepatoma (hepatocellular carcinoma), cholangiocarcinoma, hepatoblastoma, angiosarcoma, hepatocellular adenoma, hemangioma); (vi) bone (including osteogenic sarcoma (osteosarcoma), fibrosarcoma, malignant fibrous histocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chrodroma, chondroblastoma, chondromyxofibroma, osteoid osteoma and giant cell tumors); (vii) nervous system (including skull (osteoma, hemangioma, granuloma, xanthoma, osteitis deformans, meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord, neurofibroma, meningioma, glioma, sarcoma)); (viii) gynecological tissues (including uterus (endometrial carcinoma), cervix (cervical carcinoma, pre-tumor cervical dysplasia), ovaries (ovarian carcinoma [serous cystadenocarcinoma, mucinous cystadenocarcinoma], granulose-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma) vagina (clear cell carcinoma, squamous cell carcinoma, botryoid sarcoma (embryonal rhabdomyosarcoma), fallopian tubes carcinoma)); (ix) hematologic system (including blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkins's lymphoma, malignant lymphoma); (x) skin (including malignant melanoma, basal cell carcinoma, squamous cell carcinoma, Kaposi's sarcoma, moles dysplastic nevi, lipoma, angioma, dermatofibroma, keloids); and (xi) adrenal glands (including neuroblastoma).

In some embodiments of methods of the present disclosure, the cancer is non-small cell lung cancer (NSCLC), small cell lung cancer, pancreatic cancer, colorectal cancer, colon cancer, bile duct cancer, cervical cancer, bladder cancer, liver cancer or breast cancer.

In certain embodiments, there are provided methods for treating or preventing a hyperplastic or hyperproliferative disease or disorder (e.g., a cancer or a tumor) in a subject (e.g., a human) comprising administering to the subject a therapeutically effective amount of at least one KRAS-G12D inhibitor compound or composition provided herein. In some embodiments, the hyperplastic disorder is a cancer or a tumor, such as without limitation non-small cell lung cancer (NSCLC), pancreatic cancer, colorectal cancer, colon cancer, bile duct cancer, cervical cancer, bladder cancer, liver cancer or breast cancer.

Other diseases, disorders and conditions that can be treated or prevented, in whole or in part, by inhibition of KRAS-G12D activity are candidate indications for the KRAS-G12D inhibitor compounds and compositions provided herein and are encompassed by methods of the disclosure.

In some embodiments, there is further provided the use of the KRAS-G12D inhibitor compounds and compositions described herein in combination with one or more additional agents. The one or more additional agents may have some KRAS-G12D-modulating activity and/or they may function through distinct mechanisms of action. In some embodiments, such agents comprise radiation (e.g., localized radiation therapy or total body radiation therapy) and/or other treatment modalities of a non-pharmacological nature. When combination therapy is utilized, the KRAS-G12D inhibitor(s) and one additional agent(s) may be in the form of a single composition or multiple compositions, and the treatment modalities can be administered concurrently, sequentially, or through some other regimen. By way of example, in some embodiments there is provided a treatment regimen wherein a radiation phase is followed by a chemotherapeutic phase. A combination therapy can have an additive or synergistic effect.

In some embodiments, there is provided the use of a KRAS-G12D inhibitor compound or composition described herein in combination with bone marrow transplantation, peripheral blood stem cell transplantation, or other types of transplantation therapy.

In particular embodiments, there is provided the use of the inhibitors of KRAS-G12D function described herein in combination with immune checkpoint inhibitors. The blockade of immune checkpoints, which results in the amplification of antigen-specific T cell responses, has been shown to be a promising approach in human cancer therapeutics. Non-limiting examples of immune checkpoints (ligands and receptors), some of which are selectively upregulated in various types of tumor cells, that are candidates for blockade include PD1 (programmed cell death protein 1); PDL1 (PD1 ligand); BTLA (B and T lymphocyte attenuator); CTLA4 (cytotoxic T-lymphocyte associated antigen 4); TIM3 (T-cell membrane protein 3); LAG3 (lymphocyte activation gene 3); A2aR (adenosine A2a receptor A2aR); and Killer Inhibitory Receptors. Non-limiting examples of immune checkpoint inhibitors include ipilimumab, nivolumab and lambrolizumab.

In other embodiments, there are provided methods for treating a cancer in a subject, comprising administering to the subject a therapeutically effective amount of at least one KRAS-G12D inhibitor compound or composition thereof and at least one chemotherapeutic agent, such agents including, but not limited to alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nucleoside analogs (e.g., gemcitabine); nitroso ureas such as carmustine, lomustine, and streptozocin; topoisomerase 1 inhibitors (e.g., irinotecan); platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, estramustine, vinblastine, docetaxol, epothilone derivatives, and paclitaxel); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). There is also provided the use of the KRAS-G12D inhibitors in combination with other agents known in the art (e.g., arsenic trioxide) and other chemotherapeutic or anti-cancer agents that may be appropriate for treatment.

In some embodiments drawn to methods of treating cancer, the administration of a therapeutically effective amount of a KRAS-G12D inhibitor in combination with at least one chemotherapeutic agent results in a cancer survival rate greater than the cancer survival rate observed by administering either agent alone. In further embodiments drawn to methods of treating cancer, the administration of a therapeutically effective amount of a KRAS-G12D inhibitor in combination with at least one chemotherapeutic agent results in a reduction of tumor size or a slowing of tumor growth greater than reduction of the tumor size or slowing of tumor growth observed by administration of either agent alone.

In further embodiments, there are provided methods for treating or preventing cancer in a subject, comprising administering to the subject a therapeutically effective amount of at least one KRAS-G12D inhibitor compound or composition and at least one signal transduction inhibitor (STI). In a particular embodiment, the at least one STI is selected from the group consisting of bcr/abl kinase inhibitors, epidermal growth factor (EGF) receptor inhibitors, her-2/neu receptor inhibitors, and farnesyl transferase inhibitors (FTIs).

In other embodiments, there are provided methods of augmenting the rejection of tumor cells in a subject comprising administering an KRAS-G12D inhibitor compound or composition in conjunction with at least one chemotherapeutic agent and/or radiation therapy, wherein the resulting rejection of tumor cells is greater than that obtained by administering either the KRAS-G12D inhibitor, the chemotherapeutic agent or the radiation therapy alone.

In further embodiments, there are provided methods for treating cancer in a subject, comprising administering to the subject a therapeutically effective amount of at least one KRAS-G12D inhibitor and at least one anti-cancer agent other than a KRAS-G12D inhibitor. It should be understood that, as used herein, a “KRAS-G12D inhibitor” refers to compounds provided herein, e.g., a compound of Formula I, a compound of Table 1, Table 2 or Table 3, or a pharmaceutically acceptable salt, ester, hydrate, or solvate thereof, or a stereoisomer thereof, and to pharmaceutical compositions thereof.

In some embodiments, there are provided methods of treating or preventing a KRAS-G12D-associated disease, disorder or condition in a subject in need thereof, comprising administering a therapeutically effective amount of at least one KRAS-G12D inhibitor or a pharmaceutical composition thereof to the subject, such that the KRAS-G12D-associated disease, disorder or condition is treated or prevented in the subject. In some embodiments, the compound is administered in an amount effective to reverse, slow or stop the progression of a KRAS-G12D-mediated cancer in the subject.

In some embodiments, the KRAS-G12D-associated disease, disorder or condition is a KRAS-G12D related cancer, tumor or hyperplastic or hyperproliferative disorder, such as, for example and without limitation, a cancer of the cardiac system, heart, lung, gastrointestinal system, genitourinary tract, liver, bone, nervous system, brain, gynecological system, hematologic tissues, skin, or adrenal glands, as described herein. In certain embodiments, the cancer, tumor or hyperplastic or hyperproliferative disorder is non-small cell lung cancer (NSCLC), small cell lung cancer, pancreatic cancer, colorectal cancer, colon cancer, bile duct cancer, cervical cancer, bladder cancer, liver cancer or breast cancer.

In certain embodiments of methods of the disclosure, the inhibition, treatment, or prevention, in full or in part, of other diseases or disorders through degradation of KRAS-G12D protein using at least one of the compounds or compositions described herein is encompassed.

In some embodiments, methods provided herein further comprise administration of at least one additional therapeutic agent to the subject. The at least one additional therapeutic agent may be administered concomitantly or sequentially with the compound or composition described herein. In some embodiments, the at least one additional therapeutic agent is a chemotherapeutic agent or an anti-cancer agent. In an embodiment, the at least one additional therapeutic agent is an immune checkpoint inhibitor, such as, without limitation, ipilimumab, nivolumab or lambrolizumab.

In additional embodiments, methods provided herein further comprise administration of a tumor vaccine (e.g., a vaccine effective against melanoma); the tumor vaccine can comprise genetically modified tumor cells or a genetically modified cell line, including genetically modified tumor cells or a genetically modified cell line that has been transfected to express granulocyte-macrophage stimulating factor (GM-CSF). In particular embodiments, the vaccine includes one or more immunogenic peptides and/or dendritic cells.

In another broad aspect, there are provided kits comprising the compound or composition described herein. Kits may include a compound described herein, or a pharmaceutically acceptable salt, ester, hydrate, solvate or stereoisomer thereof, for use to treat, prevent or inhibit a KRAS-G12D-associated disease, disorder or condition. Kits may further comprise a buffer or excipient, and/or instructions for use. In some embodiments, kits further comprise at least one additional therapeutic agent, such as without limitation a chemotherapeutic agent, an immune- and/or inflammation-modulating agent, an anti-hypercholesterolemia agent, an anti-infective agent, or an immune checkpoint inhibitor.

### DETAILED DESCRIPTION

The number of subjects diagnosed with cancer and the number of deaths attributable to cancer continue to rise. Recent experimental evidence indicates that KRAS inhibitors, particularly KRAS-G12D inhibitors, may represent an important new treatment modality for treatment of many cancers and tumors. However, traditional treatment approaches including chemotherapy, radiotherapy and traditional enzymatic inhibitors are generally difficult for patients to tolerate and/or can become less effective as cancers and tumors evolve to circumvent such treatments.

There are provided herein, inter alia, bifunctional small molecule compounds that can inhibit KRAS-G12D, as well as compositions thereof, and methods of using the compounds and compositions for the treatment and prevention of the diseases, disorders and conditions described herein. Compounds provided herein are useful as inhibitors of KRAS-G12D and, therefore, useful in the treatment of diseases, disorders, and conditions in which KRAS-G12D activity plays a role. Specifically, compounds provided herein are proteolysis-targeting chimeras (Protacs) which can bind to a target protein of interest (KRAS-G12D) and to an E3 ligase. The compounds act to recruit the E3 ligase to the target protein (KRAS-G12D) and thereby modulate degradation of the target protein.

Without wishing to be limited by theory, Protacs can provide several advantages therapeutically compared to traditional enzymatic inhibitors. First, they need only bind to their targets with high selectivity to work (rather than inhibit the target protein's enzymatic activity). Further, previously undruggable proteins can be targeted, since a target catalytic pocket is not needed. Another advantage is that, due to their catalytic mechanism, Protacs can often be administered at lower doses compared to inhibitor analogues and traditional enzymatic inhibitor compounds. Off-target effects can also be reduced. Finally, acquired drug resistance is less likely to occur for Protacs. For example, treatment with Protacs may avoid or prevent mutation-driven drug resistance that would circumvent a traditional enzymatic inhibitor. KRAS and KRAS-G12D inhibitor compounds of the disclosure may provide one or more of these advantages compared to other KRAS and KRAS-G12D inhibitors.

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The terms “about” and “approximately” are used to indicate that a value includes an inherent variation of error for the device or the method being employed to determine the value.

The term “derivative” as used herein, is understood as being a substance similar in structure to another compound but differing in some slight structural detail.

The term “KRAS-G12D” (also referred to as “KRAS^{G12D}”) refers to a mutant form of the mammalian KRAS protein, in which the glycine residue at position 12 is replaced by an aspartic acid residue.

Ubiquitin (Ub) is a small protein that exists in all eukaryotic cells and is highly conserved throughout eukaryotic evolution, with human and yeast ubiquitin sharing 96% sequence identity. It contains 76 amino acids and has a molecular mass of about 8.6 kDa. Ubiquitin performs myriad functions through conjugation to a large range of target proteins. In general, ubiquitination affects cellular processes by regulating the degradation of proteins (via the proteasome and lysosome), coordinating the cellular localization of proteins, activating and inactivating proteins, and modulating protein-protein interactions.

The term “ubiquitylation” (also referred to as “ubiquitination” or “ubiquitinylation”) is an enzymatic post-translational modification in which a ubiquitin protein is attached to a substrate protein. In general, ubiquitination refers to the process of covalent binding of ubiquitin to a target protein under the catalysis of a series of enzymes. The ubiquitination process usually requires the cooperation of three ubiquitination enzymes: E1 ubiquitin activating enzyme, E2 ubiquitin binding enzyme, and E3 ubiquitin ligase (also referred to herein as “E3 ligase”; the terms “E3 ubiquitin ligase” and “E3 ligase” are used interchangeably herein). E3 ubiquitin ligases catalyze the final step of the ubiquitination cascade, most commonly creating an isopeptide bond between a ligand of the substrate/target protein and the C-terminal glycine of ubiquitin. Common E3 ubiquitin ligases include, for example and without limitation, VHL (Von Hippel-Lindau), CRBN (Cereblon), MDM2, c-IAP1, AhR, Nimbolide, CCW16, KB02, KEAP1, beta-TrCP1, DCAF15, DCAF16, RNF114, and others. Hundreds of E3 ubiquitin ligases are known, and it should be understood that any suitable E3 ligase may be targeted/bound by compounds of the present disclosure.

The term “proteolysis targeting chimera” or “Protac” refers to a heterobifunctional molecule, composed of two active domains and optionally a linker, which is capable of removing specific unwanted proteins. The active domains are protein-binding domains, one that binds to a target protein meant for degradation and one that binds to an E3 ubiquitin ligase. Recruitment of the E3 ligase to the target protein results in ubiquitination and subsequent degradation of the target protein via the proteasome. In this way Protacs act to induce selective intracellular proteolysis.

The term “prodrug” or its equivalent refers to a reagent that is directly or indirectly converted into an active form in vitro or in vivo (see, for example, R. B. Silverman, 1992, “The Organic Chemistry of Drug Design and Drug Action,” Academic Press, Chap. 8; Bundgaard, Hans; Editor. Neth. (1985), “Design of Prodrugs” 360 pp. Elsevier, Amsterdam; Stella, V.; Borchardt, R.; Hageman, M.; Oliyai, R.; Maag, H.; Tilley, J. (Eds.) (2007), “Prodrugs: Challenges and Rewards, XVIII, 1470 p. Springer). A prodrug can be used to change the biological distribution of specific drugs (for example, to make the drug usually not enter the protease reaction site) or its pharmacokinetics. A variety of groups have been used to modify compounds to form prodrugs, such as esters, ethers, phosphate esters/salts, etc. When a prodrug is administered to a subject, the group is cleaved in the subject by an enzymatic or non-enzymatic process, e.g., by reduction, oxidation or hydrolysis, or in another way, to release the active compound. As used herein, “prodrug” may include pharmaceutically acceptable salts or esters, or pharmaceutically acceptable solvates or chelates, as well as crystalline forms of a compound.

The terms “peptide”, “polypeptide” and “oligopeptide” refer to a compound formed by the dehydration and condensation of two or more amino acid residues, which are linked together by amide bonds. In general, the number of amino acids in a small peptide or oligopeptide is from 2 (dipeptide) to 20 (icosapeptide), although the number is not particularly limited.

The term “residue” refers to the main part of a molecule which remains after removing a certain group, such as an amino acid residue (such as the structure H₂NCH₂C(O)—, that is, the glycyl group, which is the part remaining after removing a hydroxyl group from glycine) or a peptide residue.

The present description refers to a number of chemical terms and abbreviations used by those skilled in the art. Nevertheless, definitions of selected terms are provided for clarity and consistency.

As used herein, the term “hydrocarbon” refers to an organic compound consisting entirely of hydrogen and carbon; it also refers to a group or a molecular fragment derived therefrom by removing one or more hydrogen atoms, which is also called a “hydrocarbon group”. The term “hydrocarbon group” includes saturated and unsaturated hydrocarbon groups, e.g., aliphatic and aromatic hydrocarbon group, e.g., alkyl groups, aryl groups, etc. Hydrocarbon groups may also include one or more heteroatom (atom which is not carbon or hydrogen); examples of such heterohydrocarbon groups include, without limitation, oxoalkyl groups, azalkyl groups, sulfoalkyl groups, phosphoroalkyl groups and mixed heterohydrocarbon groups with different heteroatoms. The chain length of hydrocarbon or heterohydrocarbon groups is not particularly limited but is generally from 1 to 20 carbon atoms, and heterohydrocarbon groups generally contain from 1 to 5 heteroatoms. It should be understood that the chemical valence of a heteroatom can be filled by hydrogen, oxygen, nitrogen, etc. in the corresponding bonding manner, as required.

As used herein, the term “alkyl” refers to saturated hydrocarbons having from one to thirty carbon atoms, including linear, branched, and cyclic alkyl groups. Examples of alkyl groups include, without limitation, methyl, ethyl, propyl, butyl, pentyl, hexyl, heptyl, octyl, nonyl, decyl, isopropyl, tert-butyl, sec-butyl, isobutyl, cyclopropyl, cyclopentyl, cyclohexyl, cycloheptyl, cyclooctyl, and the like. The term alkyl includes both unsubstituted alkyl groups and substituted alkyl groups. The terms “C₁-Cₙalkyl” and “C₁₋ₙ alkyl”, wherein n is an integer from 2 to 30, are used interchangeably to refer to an alkyl group having from 1 to the indicated “n” number of carbon atoms. Alkyl residues may be substituted or unsubstituted. In some embodiments, for example, alkyl may be substituted by hydroxyl, amino, carboxyl, carboxylic ester, amide, carbamate, or aminoalkyl. In some particular embodiments, “alkyl” is modified by a range of the number of carbon atoms and thus the size of the alkyl group is defined specifically. For example, a C₁₁-C₃₀ alkyl specifies an alkyl group containing at least 11 carbon atoms and not more than 30 carbon atoms.

As used herein, the term “acyclic” refers to an organic moiety without a ring system. The term “aliphatic group” includes organic moieties characterized by straight or branched-chains, typically having between 1 and 15 carbon atoms. Aliphatic groups include non-cyclic alkyl groups, alkenyl groups, and alkynyl groups.

As used herein, the term “alkenyl” refers to unsaturated hydrocarbons having from two to thirty carbon atoms, including linear, branched, and cyclic non aromatic alkenyl groups, and comprising between one to six carbon-carbon double bonds. Examples of alkenyl groups include, without limitation, vinyl, allyl, 1-propen-2-yl, 1-buten-3-yl, 1-buten-4-yl, 2-buten-4-yl, 1-penten-5-yl, 1,3-pentadien-5-yl, cyclopentenyl, cyclohexenyl, ethylcyclopentenyl, ethylcylohexenyl, and the like. The term alkenyl includes both unsubstituted alkenyl groups and substituted alkenyl groups. The terms “C₂-Cₙalkenyl” and “C₂₋ₙ alkenyl”, wherein n is an integer from 3 to 30, are used interchangeably to refer to an alkenyl group having from 2 to the indicated “n” number of carbon atoms. In some particular embodiments, “alkenyl” is modified by a range of the number of carbon atoms and thus the size of the alkenyl group is defined specifically. For example, a C₁₁-C₃₀ alkenyl specifies an alkenyl group containing at least 11 carbon atoms and not more than 30 carbon atoms.

As used herein, the term “alkynyl” refers to unsaturated hydrocarbons having from two to thirty carbon atoms, including linear, branched, and cyclic non aromatic alkynyl groups, and comprising between one to six carbon-carbon triple bonds. Examples of alkynyl groups include, without limitation, ethynyl, 1-propyn-3-yl, 1-butyn-4-yl, 2-butyn-4-yl, 1-pentyn-5-yl, 1,3-pentadiyn-5-yl, and the like. The term alkynyl includes both unsubstituted alkynyl groups and substituted alkynyl groups. The terms “C₂-Cₙalkynyl” and “C₂₋ₙ alkynyl”, wherein n is an integer from 3 to 30, are used interchangeably to refer to an alkynyl group having from 2 to the indicated “n” number of carbon atoms. In some particular embodiments, “alkynyl” is modified by a range of the number of carbon atoms and thus the size of the alkynyl group is defined specifically. For example, a C₁₁-C₃₀ alkynyl specifies an alkynyl group containing at least 11 carbon atoms and not more than 30 carbon atoms.

Unless the number of carbons is otherwise specified, “lower” as in “lower aliphatic,” “lower alkyl,” “lower alkenyl,” and “lower alkylnyl”, as used herein means that the moiety has at least one (two for alkenyl and alkynyl) and equal to or less than 6 carbon atoms.

The terms “cycloalkyl”, “alicyclic”, “carbocyclic”, “cyclic group”, “alicyclic group”, “cyclic hydrocarbon group” and equivalent expressions refer to a group comprising a saturated or partially unsaturated carbocyclic ring in a single, spiro (sharing one atom), or fused (sharing at least one bond) carbocyclic ring system having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbomyl, and the like. The term cycloalkyl includes both unsubstituted cycloalkyl groups and substituted cycloalkyl groups. The terms “C₃-Cₙcycloalkyl” and “C₃₋ₙ cycloalkyl”, wherein n is an integer from 4 to 15, are used interchangeably to refer to a cycloalkyl group having from 3 to the indicated “n” number of carbon atoms in the ring structure. Unless the number of carbons is otherwise specified, “lower cycloalkyl” groups as herein used, have at least 3 and equal to or less than 8 carbon atoms in their ring structure.

Cycloalkyl residues can be saturated or contain one or more double bonds within the ring system. In particular they can be saturated or contain one double bond within the ring system. In unsaturated cycloalkyl residues the double bonds can be present in any suitable positions. Monocycloalkyl residues are, for example, cyclopropyl, cyclobutyl, cyclopentyl, cyclopentenyl, cyclohexyl, cyclohexenyl, cycloheptyl, cycloheptenyl, cyclooctyl, cyclononyl, cyclodecyl, cycloundecyl, cyclododecyl or cyclotetradecyl, which can also be substituted, for example by C₁₋₄ alkyl. Examples of substituted cycloalkyl residues are 4-methylcyclohexyl and 2,3-dimethylcyclopentyl. Examples of parent structures of bicyclic ring systems are norbornane, bicyclo[2.2.1]heptane, bicyclo[2.2.2]octane and bicyclo[3.2.1]octane.

The term “heterocycloalkyl” and equivalent expressions refers to a group comprising a saturated or partially unsaturated carbocyclic ring in a single, spiro (sharing one atom), or fused (sharing at least one bond) carbocyclic ring system having from three to fifteen ring members, including one to six heteroatoms (e.g., N, O, S, P) or groups containing such heteroatoms (e.g., NH, NRx (Rx is alkyl, acyl, aryl, heteroaryl or cycloalkyl), PO₂, SO, SO₂, and the like). Heterocycloalkyl groups may be C-attached or heteroatom-attached (e.g., via a nitrogen atom) where such is possible. Examples of heterocycloalkyl groups include, without limitation, pyrrolidino, tetrahydrofuranyl, tetrahydrodithienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3,1,0]hexanyl, 3-azabicyclo[4,1,0]heptanyl, 3H-indolyl, quinolizinyl, and sugars, and the like. The term heterocycloalkyl includes both unsubstituted heterocycloalkyl groups and substituted heterocycloalkyl groups. The terms “C₃-Cₙheterocycloalkyl” and “C₃₋ₙ heterocycloalkyl”, wherein n is an integer from 4 to 15, are used interchangeably to refer to a heterocycloalkyl group having from 3 to the indicated “n” number of atoms in the ring structure, including at least one hetero group or atom as defined above. Unless the number of carbons is otherwise specified, “lower heterocycloalkyl” groups as herein used, have at least 3 and equal to or less than 8 carbon atoms in their ring structure.

The terms “aryl” and “aryl ring” refer to aromatic groups having “4n+2” (pi) electrons, wherein n is an integer from 1 to 7, in a conjugated monocyclic or polycyclic system (fused or not) and having six to fourteen ring atoms. In certain embodiments, n is an integer from 1 to 3. A polycyclic ring system includes at least one aromatic ring. Aryl may be directly attached, or connected via a C₁-C₃ alkyl group or a C₁-C₆ alkyl group (also referred to as arylalkyl or aralkyl). Examples of aryl groups include, without limitation, phenyl, benzyl, phenethyl, 1-phenylethyl, tolyl, naphthyl, biphenyl, triphenyl, terphenyl, indenyl, benzocyclooctenyl, benzocycloheptenyl, benzocycloheptyl, azulene, acenaphthene, azulenyl, acenaphthylenyl, fluorenyl, phenanthernyl, anthracene, anthracenyl, and the like. The term aryl includes both unsubstituted aryl groups and substituted aryl groups. The terms “C₆-Cₙaryl” and “C₆₋ₙ aryl”, wherein n is an integer from 6 to 30, are used interchangeably to refer to an aryl group having from 6 to the indicated “n” number of atoms in the ring structure, including at least one hetero group or atom as defined above. When the aryl group is connected to an alkyl group, the entire group is known as arylalkyl group or alkylaryl group.

The terms “heteroaryl” and “heteroaryl ring” refer to an aromatic group having “4n+2”(pi) electrons, wherein n is an integer from 1 to 7, in a conjugated monocyclic or polycyclic system (fused or not) and having five to fourteen ring members, including one to six heteroatoms (e.g. N, O, S) or groups containing such heteroatoms (e.g. NH, NRx (Rx is alkyl, acyl, aryl, heteroaryl or cycloalkyl), SO, and the like). A polycyclic ring system includes at least one heteroaromatic ring. Heteroaryls may be directly attached, or connected via a C₁-C₃alkyl group (also referred to as heteroarylalkyl or heteroaralkyl). Heteroaryl groups may be C-attached or heteroatom-attached (e.g., via a nitrogen atom), where such is possible. Examples of heteroaryl groups include, without limitation, pyridyl, imidazolyl, pyrimidinyl, pyrazolyl, triazolyl, tetrazolyl, furyl, thienyl; isooxazolyl, thiazolyl, oxazolyl, isothiazolyl, pyrrollyl, quinolinyl, isoquinolinyl, indolyl, isoindolyl, chromenyl, isochromenyl, benzimidazolyl, benzofuranyl, cinnolinyl, indazolyl, indolizinyl, phthalazinyl, pyridazinyl, pyrazinyl, triazinyl, isoindolyl, pteridinyl, purinyl, oxadiazolyl, thiadiazolyl, furazanyl, benzofurazanyl, benzothiophenyl, benzothienyl, benzothiazolyl, benzoxazolyl, quinazolinyl, quinolizinyl, quinolonyl, isoquinolonyl, quinoxalinyl, naphthyridinyl, furopyridinyl, carbazolyl, phenanthridinyl, acridinyl, perimidinyl, phenanthrolinyl, phenazinyl, phenothiazinyl, phenoxazinyl, dibenzofurnayl, and the like. The term heteroaryl includes both unsubstituted heteroaryl groups and substituted heteroaryl groups. The terms “C₅-Cₙheteroaryl” and “C₅₋ₙ heteroaryl”, wherein n is an integer from 6 to 29, are used interchangeably to refer to a heteroaryl group having from 5 to the indicated “n” number of atoms in the ring structure, including at least one hetero group or atom as defined above.

The term “heterocycle” or “heterocyclic” and equivalent expressions used herein refer to groups containing a saturated or partially unsaturated carbon ring in a single, spiral (sharing one atom) or fused (sharing at least one bond) carbon ring system, which has from 3 to 15 carbon atoms, including from 1 to 6 heteroatoms (such as N, O, S, P etc.) or containing heteroatoms such as, without limitation, NH, NRx (where Rx is alkyl, acyl, aryl, heteroaryl or cycloalkyl), PO₂, SO, SO₂, etc.). Heterocyclic hydrocarbon groups can be connected with C or with heteroatoms (for example, through nitrogen atoms).

The terms “heterocycle” or “heterocyclic” include heterocyclic alkyl and heteroaryl groups. Examples of heterocycles include, without limitation, acridine, acrine, azocinyl, benzimidazolyl, benzodihydropyranyl, benzofuranyl, benzothiofuranyl, benzothiophenyl, benzoxazolyl, benzothiazolyl, benzotriazolyl, benzotetrazolyl, benzoisoxazolyl, benzoisothiazolyl, benzimidazolinyl, carbazolyl, 4αH-carbazolyl, carbolinyl, chromanyl, chromonyl, chromenyl, cinnolinyl, decahydroquinolinyl, 2H,6H-1,5,2-dithiazinyl, dihydroindolyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, misolinyl, morpholinyl, naphthyridinyl, naphthyridyl, octahydroisoquinolinyl, oxadiazolyl, 1,2,4-oxadiazole, 1,2,5-oxadiazole, 1,3,4-oxadiazole, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinothiazole, pyridinyl, pyridyl, pyrimidinyl, pyrrolidinyl, pyrrolinyl, 2H-pyrrolyl, pyrrolyl, quinazolyl, quinazolinyl, quinolinyl, 4H-quinolizinyl, quinoxalinyl, quinuclidinyl, 4H-quinazinyl, quinoxalinyl, quinine cyclo, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiaanthracyl, thiophenothiazolyl, thiophenoxazolyl, thiopheno imidazolyl, thiophenyl, triazinyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl, and the like. The term heterocycle includes both unsubstituted heterocyclic groups and substituted heterocyclic groups. The terms “heterocyclic hydrocarbon group” and “heterocyclic alkyl group” refer to the combined group of heterocyclic and hydrocarbon/alkyl groups.

The term “amine” or “amino,” as used herein, refers to an unsubstituted or substituted moiety of the formula —NRaRb, in which Ra and Rb are each independently hydrogen, alkyl, aryl, or heterocyclyl, or Ra and Rb, taken together with the nitrogen atom to which they are attached, form a heterocyclic ring. For example, an amine or amino may be an unsubstituted or substituted fragment of a general formula —N, including —NH₂, —NHR, or —NRR′, where R and R′ are the same or different and are substituted or unsubstituted and saturated or unsaturated alkyl or hydrocarbon groups. The term amino includes compounds or moieties in which a nitrogen atom is covalently bonded to at least one carbon or heteroatom. Thus, the terms “alkylamino” and “dialkylamino” as used herein mean an amine group having respectively one and at least two C₁-C₆alkyl groups attached thereto. The terms “arylamino” and “diarylamino” include groups wherein the nitrogen is bound to at least one or two aryl groups, respectively. The terms “amide”, “amide group” or “aminocarbonyl” include compounds or moieties which contain a nitrogen atom which is bound to the carbon of a carbonyl or a thiocarbonyl group. For example, an amide group may have the structure —C(═O)NH₂, —C(═O)NHR, or —C(═O)NRR′, in which the amino group is directly connected to the acyl group. The term “acyl hydrocarbon group” or “acyl alkyl group” refers to the combined group of acyl and hydrocarbon/alkyl, in which the carbon atom of the acyl group is connected to the hydrocarbon/alkyl group. The term “acylamino” refers to an amino group directly attached to an acyl group as defined herein.

The term “bicycle” or “bicyclic” refers to a ring system with two rings that has two ring carbon atoms in common, and which can be located at any position along either ring, generally referring to bicyclic hydrocarbon radical, bicyclic aromatic carbon atom ring structure radical, and a saturated or partially unsaturated bicyclic carbon atom ring structure radical in which one or more carbon atom ring members have been replaced, where allowed by structural stability, with a heteroatom, such as an O, S or N atom. The bicyclic system can be a fused-ring system, such as bicyclo[4.4.0]decane or naphthalene, or a bridged-ring system, such as bicyclo[2.2.2]octane.

The term “tricycle” or “tricyclic” refers to a ring system with three rings that has three ring carbon atoms in common, and which can be located at any position along each ring; generally referring to tricyclic hydrocarbon radical, tricyclic aromatic carbon atom ring structure radical, and a saturated or partially unsaturated tricyclic carbon atom ring structure radical in which one or more carbon atom ring members have been replaced, where allowed by structural stability, with a heteroatom, such as an O, S or N atom. A tricyclic system can have three rings arranged as a fused ring, such as anthracene or tetradecahydroanthracene, or a bridged ring, such as in adamantine or tricycle[3.3.1.1]decane.

The term “multi-cycle”, “multicycle”, “multi-cyclic”, or “multi-cyclic” means a ring system with more than three rings having more than three ring carbon atoms in common, and which can be located at any position along either ring. The term generally refers to a multicyclic hydrocarbon radical, a multicyclic aromatic carbon atom ring structure radical, and a saturated or partially unsaturated multicyclic carbon atom ring structure radical in which one or more carbon atom ring members have been replaced, where allowed by structural stability, with a heteroatom, such as an O, S or N atom.

The term “fused ring” or “fused” refers to a polycyclic ring system that contains fused rings. Typically, a fused ring system contains 2 or 3 rings and/or up to 18 ring atoms. As defined above, cycloalkyl radicals, aryl radicals and heterocyclyl radicals may form fused ring systems. Thus, a fused ring system may be aromatic, partially aromatic or not aromatic and may contain heteroatoms. A spiro ring system is not a fused-polycyclic by this definition, but fused polycyclic ring systems of the invention may themselves have spiro rings attached thereto via a single ring atom of the system. The term “benzo-fused ring” refers to a fused ring system in which at least one of the rings is a benzene ring. [Please confirm the definition of benzo-fused ring is correct] Examples of fused ring systems include, but are not limited to, naphthyl (e.g. 2-naphthyl), indenyl, fenanthryl, anthracyl, pyrenyl, benzimidazole, benzothiazole, etc. The terms “fused ring” and “fused-cyclic” are used interchangeably herein.

The term “spiral ring” or “spiral” refers to an organic compound, that presents a twisted structure of two or more rings (a ring system), in which 2 or 3 rings are linked together by one common atom. Spiro compounds may be fully carbocyclic (all carbon), such as without limitation spiro[5.5]undecane or heterocyclic (having one or more non-carbon atom), including but not limited to carbocyclic spiro compounds, heterocyclic spiro compounds and polyspiro compounds. The terms “spiral ring” and “spiral-cyclic” are used interchangeably herein.

The term “bridged ring” or “bridged” refers to a carbocyclic or heterocyclic moiety where two or more atoms are shared between two or more ring structures, where any such shared atom is C, N, S, or other heteroatom arranged in a chemically reasonable substitution pattern. Alternatively, a “bridged” compound also refers to a carbocyclic or heterocyclic ring structure where one atom at any position of a primary ring is bonded to a second atom on the primary ring through either a chemical bond or atom (s) other than a bond which does (do) not comprise a part of the primary ring structure. The first and second atom may or may not be adjacent to one another in the primary ring. Illustrated below are specific non-limiting examples of bridged ring structures contemplated herein. Other carbocyclic or heterocyclic bridged ring structures are also contemplated, including bridged rings wherein the bridging atoms are C or heteroatom (s) arranged in chemically reasonable substitution patterns, as are known in the art.

The term “nitro” means —NO₂; the terms “halo” and “halogen” refer to bromine, chlorine, fluorine or iodine substituents; the terms “thiol”, “thio”, and “mercapto” mean —SH; and the terms “hydroxyl” and “hydroxy” mean —OH. The term “alkylthio” refers to an alkyl group, having a sulfhydryl group attached thereto. Suitable alkylthio groups include groups having 1 to about 12 carbon atoms, preferably from 1 to about 6 carbon atoms. The term “alkylcarboxyl” as used herein means an alkyl group having a carboxyl group attached thereto.

The terms “alkoxy” and “lower alkoxy” as used herein mean an alkyl group having an oxygen atom attached thereto. Representative alkoxy groups include groups having 1 to about 6 carbon atoms, e.g., methoxy, ethoxy, propoxy, tert-butoxy and the like. Examples of alkoxy groups include but are not limited to methoxy, ethoxy, isopropyloxy, propoxy, butoxy, pentoxy, fluoromethoxy, difluoromethoxy, trifluoromethoxy, chloromethoxy, dichloromethoxy, trichloromethoxy groups, and the like. The term “alkoxy” includes both unsubstituted and substituted alkoxy groups, etc., as well as halogenated alkoxy/perhalogenated alkyloxy groups. Similarly, the term “hydrocarboxy” or “oxyhydrocarboxy” refers to the group or structure where the hydrocarbon group is connected to the oxygen atom. Lower alkoxy means the alkyl group in the alkoxy is a lower alkyl group.

The terms “carbonyl” and “carboxy” include compounds and moieties which contain a carbon connected with a double bond to an oxygen atom (C(═O)). “Carbonyl” is the component of functional groups such as aldehydes, ketones, and carboxylic acids. Examples of moieties which contain a carbonyl include aldehydes, ketones, carboxylic acids, amides, esters, anhydrides, etc.

The term “acyl” refers to a carbonyl group that is attached through its carbon atom to a hydrogen (i.e., formyl), an aliphatic group (e.g., C₁-C₂₉ alkyl, C₁-C₂₉ alkenyl, C₁-C₂₉ alkynyl, e.g., acetyl), a cycloalkyl group (C₃-C₈cycloalkyl), a heterocyclic group (C₃-C₈heterocycloalkyl and C₅-C₆heteroaryl), an aromatic group (C₆aryl, e.g., benzoyl), and the like. Acyl groups may be unsubstituted or substituted acyl groups (e.g., salicyloyl).

The term “amidoalkyl” or “hydrocarbonamide/alkylamide” refers to the group formed by the combination of hydrocarbon/alkyl group and amide group. The term “acyl hydrocarbon group” or “hydrocarbonyl group” refers to the group formed by the combination of hydrocarbon group and acyl group. The term “carbonyl hydrocarbon group” or “hydrocarbon carbonyl group” refers to the group formed by the combination of hydrocarbon group and carbonyl group.

It should be understood that “substitution” or “substituted with” includes the implicit proviso that such substitution is in accordance with the permitted valence of the substituted atom and the substituent, and that the substitution results in a stable compound, i.e., a compound which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more. The term “substituted”, when used in association with any of the foregoing groups refers to a group substituted at one or more position with substituents such as acyl, amino (including simple amino, mono and dialkylamino, mono and diarylamino, and alkylarylamino), acylamino (including carbamoyl, and ureido), alkylcarbonyloxy, arylcarbonyloxy, alkoxycarbonyloxy, alkoxycarbonyl, carboxy, carboxylate, aminocarbonyl, mono and dialkylaminocarbonyl, cyano, azido, halogen, hydroxyl, nitro, trifluoromethyl, thio, alkylthio, arylthio, alkylthiocarbonyl, thiocarboxylate, lower alkyl, lower alkenyl, lower alkynyl, cycloalkyl, heterocycloalkyl, aryl, heteroaryl, lower alkoxy, aryloxy, aryloxycarbonyloxy, benzyloxy, benzyl, sulfinyl, alkylsulfinyl, sulfonyl, sulfate, sulfonate, sulfonamide, phosphate, phosphonato, phosphinato, oxo, guanidine, imino, formyl and the like. Any of the above substituents can be further substituted if permissible, e.g., if the group contains an alkyl group, an aryl group, or other.

The terms “substituted”, “with substituent” and “with substitution” mean that the parent compound or part thereof has at least one substituent group. Unless otherwise indicated, a “substituent” group can be at one or more substitutable positions of the parent group, and when there is more than one substituent present at different positions of a given structure, the substituents can be the same or different at each position. In certain embodiments, the terms “substituent” and “substituted group” include, but are not limited to, halogen (F, Cl, Br or I), hydroxyl, mercapto, amino, nitro, carbonyl, carboxyl, alkyl, alkoxy, alkylamino, aryl, aryloxy, arylamino, acyl, sulfinyl, sulfonyl, phosphonyl and other organic parts routinely used and accepted in organic chemistry.

Where multiple substituents are indicated as being attached to a structure, it is to be understood that the substituents can be the same or different. Thus for example “Rₘ optionally substituted with 1, 2 or 3 R_{q} groups” indicates that Rₘ is substituted with 1, 2, or 3 R_{q} groups where the R_{q} groups can be the same or different.

The terms “unsubstituted” and “without substitution” mean that a compound or part thereof has no substituent except the undetermined chemical saturation of hydrogen atom.

The term “solvate” refers to a physical association of a compound with one or more solvent molecules, whether organic or inorganic. This physical association includes hydrogen bonding. In certain instances, a solvate will be capable of isolation, for example when one or more solvent molecules are incorporated in the crystal lattice of a crystalline solid. “Solvate” encompasses both solution-phase and isolable solvates. Exemplary solvates include, without limitation, hydrates, ethanolates, methanolates, hemiethanolates, and the like.

The term “hydrate” refers to a compound that is bonded to one or more water (H₂O) molecule, e.g., by a hydrogen bond.

The term “pharmaceutically acceptable” as used herein refers to drugs, medicaments, inert ingredients etc., which the term describes, suitable for use in contact with the tissues of humans and lower animals without undue toxicity, incompatibility, instability, irritation, allergic response, and the like, commensurate with a reasonable benefit/risk ratio.

A “pharmaceutically acceptable salt” of a compound means a salt of a compound that is pharmaceutically acceptable. Desirable are salts of a compound that retain or improve the biological effectiveness and properties of the free acids and bases of the parent compound as defined herein or that take advantage of an intrinsically basic, acidic or charged functionality on the molecule and that are not biologically or otherwise undesirable. Examples of pharmaceutically acceptable salts are also described, for example, in Berge et al., “Pharmaceutical Salts”, J. Pharm. Sci. 66, 1-19 (1977). Non-limiting examples of such salts include:

(1) acid addition salts, formed on a basic or positively charged functionality, by the addition of inorganic acids such as hydrochloric acid, hydrobromic acid, hydroiodic acid, sulfuric acid, sulfamic acid, nitric acid, phosphoric acid, carbonate forming agents, and the like; or formed with organic acids such as acetic acid, propionic acid, lactic acid, oxalic, glycolic acid, pivalic acid, t-butylacetic acid, O-hydroxybutyric acid, valeric acid, hexanoic acid, cyclopentanepropionic acid, pyruvic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, cyclohexylaminosulfonic acid, benzenesulfonic acid, sulfanilic acid, 4-chlorobenzenesulfonic acid, 2-napthalenesulfonic acid, 4-toluenesulfonic acid, camphorsulfonic acid, 3-phenyl propionic acid, lauryl sulphonic acid, lauryl sulfuric acid, oleic acid, palmitic acid, stearic acid, lauric acid, embonic (pamoic) acid, palmoic acid, pantothenic acid, lactobionic acid, alginic acid, galactaric acid, galacturonic acid, gluconic acid, glucoheptonic acid, glutamic acid, naphthoic acid, hydroxynapthoic acid, salicylic acid, ascorbic acid, stearic acid, muconic acid, and the like;

(2) base addition salts, formed when an acidic proton present in the parent compound either is replaced by a metal ion, including, an alkali metal ion (e.g., lithium, sodium, potassium), an alkaline earth ion (e.g., magnesium, calcium, barium), or other metal ions such as aluminum, zinc, iron and the like; or coordinates with an organic base such as ammonia, ethylamine, diethylamine, ethylenediamine, N,N′-dibenzylethylenediamine, ethanolamine, diethanolamine, triethanolamine, tromethamine, N-methylglucamine, piperazine, chloroprocain, procain, choline, lysine and the like.

Pharmaceutically acceptable salts may be synthesized from a parent compound that contains a basic or acidic moiety, by conventional chemical methods. Generally, such salts are prepared by reacting the free acid or base forms of compounds with a stoichiometric amount of the appropriate base or acid in water or in an organic solvent, or in a mixture of the two. Salts may be prepared in situ, during the final isolation or purification of a compound or by separately reacting a compound in its free acid or base form with the desired corresponding base or acid, and isolating the salt thus formed. The term “pharmaceutically acceptable salts” also include zwitterionic compounds containing a cationic group covalently bonded to an anionic group, as they are “internal salts”. It should be understood that all acid, salt, base, and other ionic and non-ionic forms of compounds described herein are intended to be encompassed. For example, if a compound is shown as an acid herein, the salt forms of the compound are also encompassed. Likewise, if a compound is shown as a salt, the acid and/or basic forms are also encompassed.

The term “ester” as used herein refers to a group or segment that can be represented by the general formula —RCOOR′. Usually, the group can be obtained by the reaction of carboxylic acid and alcohol (elimination of a molecule of water). Non-limiting examples for —R— include a lower alkyl or aryl, such as methylene, ethylene, isopropylene, phenylene, benzylene, etc. Non-limiting examples for R′ include a lower alkyl or aryl, such as methyl, ethyl, propyl, isopropyl, butyl, phenyl, benzyl, etc. The term “ester alkyl” means that R′ is an alkyl, one end of which is directly connected with the oxygen on the ester, and the other end is covalently bonded with at least one carbon or heteroatom in a compound or fragment.

As used herein, a “stereoisomer” of a compound refers to the isomer produced by the different spatial arrangement of atoms or groups in a molecule. Isomers caused by the same order of atoms or atomic groups in the molecule but with different spatial arrangement are called stereoisomers. Stereoisomers are mainly divided into two categories: stereoisomers caused by bond length, bond angle, intramolecular double bond, ring, and the like are called configuration stereoisomers. In general, isomers cannot or are difficult to convert into each other. Stereoisomers caused only by the rotation of a single bond are called conformational stereoisomers, sometimes also known as rotational isomers. When the rotation in the rotating isomer is blocked and cannot rotate, it becomes a “stereoisomer”, for example, in the biphenyl structure, when α- and α′-positions bear large and different substituents, the rotation of the single bond between the two phenyl rings stops due to the hindrance between the substituents, producing two stereoisomers.

### Compounds

In certain embodiments, there are provided bifunctional compounds, and/or pharmaceutically acceptable salts, esters, hydrates, solvates, and stereoisomers thereof, comprising a KRAS-G12D protein targeting group (W) and an E3 ligase binding group (T). In some such embodiments, bifunctional compounds of the disclosure further comprise a bivalent linking group that connects W and T together via a covalent linkage. In alternative embodiments, the linking group is absent and W and T are connected together directly.

Unless specified otherwise, the terms W and T are used herein with their inclusive meanings. For example, the term W includes all groups or parts of a structure that may target or recognize the KRAS-G12D protein; it may be an independent molecule or group that binds KRAS-G12D protein, or, alternatively, a group that combines with other molecules or structures to recognize the target protein. W is therefore intended to include all molecules or groups that can be used, alone or in combination with other molecules, to recognize KRAS-G12D protein, partially or completely. Similarly, the term T includes all groups or parts of a structure that may be used to bind to an E3 ubiquitin ligase (such as, without limitation, a ligand of an E3 ligase or a portion thereof). T is therefore intended to include all molecules or groups that can be used, alone or in combination with other molecules, to bind to an E3 ubiquitin ligase, partially or completely.

Further, it should be understood that the number and the position of W and T groups in a compound of the disclosure are provided for illustration purposes only, and are not intended to be particularly limited. A compound may include more than one W and/or T group, and groups may be connected together in different orientations and positions, as long as the bifunctional compound can still act to inhibit the target protein, e.g., by binding to the target protein and the E3 ligase and modulating degradation of the target protein.

In certain embodiments of bifunctional compounds of the disclosure, the KRAS-G12D protein targeting group (W) and the E3 ligase binding group (T) are connected directly to each other. In alternative embodiments, bifunctional compounds of the disclosure comprise a bivalent linking group (L) that connects the KRAS-G12D protein targeting group (W) and the E3 ligase binding group (T) together. The structure of L is not particularly limited, and structures provided herein are exemplary only and not intended to limit the scope of L. In general, when L is present in a bifunctional compound of the disclosure, it can be any bivalent structural fragment, i.e., having at least two connecting points, which can connect W and T covalently to form a bifunctional compound.

As used herein, the term “compounds of the disclosure” and equivalent expressions refers to bifunctional compounds provided herein as being useful for at least one purpose of the disclosure, e.g., those encompassed by structural Formula (I), and includes specific compounds mentioned herein such as those in Tables 1-2 as well as their pharmaceutically acceptable salts, esters, hydrates, solvates and stereoisomers.

As would be understood by a person of ordinary skill in the art, the recitation of “a compound” is intended to include salts, esters, solvates, hydrates, oxides, and inclusion complexes of that compound as well as any stereoisomeric form or polymorphic form, or a mixture of any such forms of that compound in any ratio. Thus, in accordance with some embodiments, a compound as described herein, including in the contexts of pharmaceutical compositions and methods of treatment, is provided as the salt form.

It should be understood that compounds described herein may contain one or more chiral centers and/or double bonds and therefore, may exist as stereoisomers, such as double-bond isomers (i.e., geometric isomers), enantiomers, or diastereomers. Chemical structures disclosed herein are intended to encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan, e.g., chiral chromatography (such as chiral HPLC), immunoassay techniques, or the use of covalently (such as Mosher's esters) and non-covalently (such as chiral salts) bound chiral reagents to respectively form a diastereomeric mixture which can be separated by conventional methods, such as chromatography, distillation, crystallization or sublimation, the chiral salt or ester is then exchanged or cleaved by conventional means, to recover the desired isomers. The compounds may also exist in several tautomeric forms including the enol form, the keto form, and mixtures thereof. The chemical structures depicted herein are also intended to encompass all possible tautomeric forms of the illustrated compounds.

Compounds may exist in unsolvated forms as well as solvated forms, including hydrated forms. In general, compounds may be hydrated or solvated. Certain compounds may exist in multiple crystalline or amorphous forms. In general, all physical forms are intended to be encompassed herein.

Compounds described herein include, but are not limited to, their optical isomers, racemates, and other mixtures thereof. In those situations, the single enantiomers or diastereomer, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, such compounds include Z- and E-forms (or cis- and trans-forms) of compounds with carbon-carbon double bonds. Where compounds described herein exist in various tautomeric forms, the term “compound” is intended to include all tautomeric forms of the compound. Such compounds also include crystal forms including polymorphs and clathrates. Similarly, the term “salt” is intended to include all tautomeric forms and crystal forms of the compound.

The configuration of any carbon-carbon double bond appearing herein is selected for convenience only and is not intended to designate a particular configuration; thus a carbon-carbon double bond depicted arbitrarily herein as E may be Z, E, or a mixture of the two in any proportion.

For compounds provided herein, it is intended that, in some embodiments, salts thereof are also encompassed, including pharmaceutically acceptable salts. Those skilled in the art will appreciate that many salt forms (e.g., TFA salt, tetrazolium salt, sodium salt, potassium salt, etc,) are possible; appropriate salts are selected based on considerations known in the art. The term “pharmaceutically acceptable salt” refers to salts prepared from pharmaceutically acceptable non-toxic acids or bases including inorganic acids and bases and organic acids and bases. For example, for compounds that contain a basic nitrogen, salts may be prepared from pharmaceutically acceptable non-toxic acids including inorganic and organic acids. Suitable pharmaceutically acceptable acid addition salts for the compounds of the present invention include without limitation acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include without limitation metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine.

For compounds provided herein, it is intended that, in some embodiments, compounds may contain unnatural proportions of atomic isotopes at one or more of the atoms that constitute such compounds. Unnatural proportions of an isotope may be defined as ranging from the amount found in nature to an amount consisting of 100% of the atom in question. For example, compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the invention may find additional utility, including but not limited to, as diagnostic and/or imaging reagents, or as cytotoxic/radiotoxic therapeutic agents. Additionally, isotopic variants can have altered pharmacokinetic and pharmacodynamic characteristics which can contribute to enhanced safety, tolerability or efficacy during treatment. All isotopic variations of compounds provided herein, whether radioactive or not, are intended to be encompassed herein.

Isotopic enrichment is a process by which the relative abundance of the isotopes of a given element are altered, thus producing a form of the element that has been enriched (i.e., increased) in one particular isotope and reduced or depleted in its other isotopic forms. As used herein, an “isotope-enriched” compound or derivative refers to a compound in which one or more specific isotopic form has been increased, i.e., one or more of the elements has been enriched (i.e., increased) in one or more particular isotope. Generally, in an isotope-enriched compound or derivative, a specific isotopic form of an element at a specific position of the compound is increased. It should be understood however that isotopic forms of two or more elements in the compound may be increased. Further, an isotope-enriched compound may be a mixture of isotope-enriched forms that are enriched for more than one particular isotope, more than one element, or both. As used herein, an “isotope-enriched” compound or derivative possesses a level of an isotopic form that is higher than the natural abundance of that form. The level of isotope-enrichment will vary depending on the natural abundance of a specific isotopic form. In some embodiments, the level of isotope-enrichment for a compound, or for an element in a compound, may be from about 2 to about 100 molar percent (%), e.g., about 2%, about 5%, about 17%, about 30%, about 51%, about 83%, about 90%, about 95%, about 96%, about 97%, about 98%, greater than about 98%, about 99%, or 100%.

As used herein, an “element of natural abundance” and an “atom of natural abundance” refers to the element or atom respectively having the atomic mass most abundantly found in nature. For example, hydrogen of natural abundance is ¹H (protium); nitrogen of natural abundance is ¹⁴N; oxygen of natural abundance is ¹⁶O; carbon of natural abundance is ¹²C; and so on. A “non-isotope enriched” compound is a compound in which all the atoms or elements in the compound are isotopes of natural abundance, i.e., all the atoms or elements have the atomic mass most abundantly found in nature.

### Compositions

In certain embodiments, there are provided pharmaceutical compositions comprising a compound of the disclosure, e.g., a compound of Formula (I), or a pharmaceutically acceptable salt, ester, hydrate, solvate or stereoisomer thereof, and a pharmaceutically acceptable excipient, carrier or diluent. In an embodiment, there is provided a pharmaceutical composition comprising a compound of Formula (I) or a compound in any one of Tables 1-2, or a pharmaceutically acceptable salt thereof, and a pharmaceutically acceptable excipient, carrier, or diluent.

The preparation of pharmaceutical compositions can be carried out as known in the art (see, for example, Remington: The Science and Practice of Pharmacy, 20^{th} Edition, 2000). For example, a therapeutic compound and/or composition, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical inhuman or veterinary medicine. Pharmaceutical preparations can also contain additives, of which many are known in the art, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents, dispersants and dispensing agents, depending on the nature of the mode of administration and dosage forms. It should be understood that, as used herein, a pharmaceutical composition comprises a compound disclosed herein (or a pharmaceutically acceptable salt, ester, hydrate, solvate, or stereoisomer thereof) and a pharmaceutically acceptable excipient, carrier, diluent, adjuvant, or vehicle. In certain embodiments, the amount of a compound in a composition is such that it is effective as an inhibitor of KRAS-G12D in a biological sample (e.g., in a cellular assay, in an in vivo model, etc.) or in a subject. In certain embodiments, the composition is formulated for administration to a subject in need of such composition. In some embodiments, the composition is an injectable formulation. In other embodiments, the composition is formulated for oral administration to a subject.

The term “pharmaceutically acceptable carrier” is used to mean any carrier, diluent, adjuvant, excipient, or vehicle, as described herein. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

A pharmaceutical composition provided herein can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, creams, tinctures, sprays or transdermal therapeutic systems, or the inhalative administration in the form of nasal sprays or aerosol mixtures, or, for example, microcapsules, implants or wafers.

In some embodiments, pharmaceutical compositions provided herein are suitable for oral administration. For example, a pharmaceutical composition may be in the form of a hard shell gelatin capsule, a soft shell gelatin capsule, a cachet, a pill, a tablet, a lozenge, a powder, a granule, a pellet, a pastille, or a dragee. Alternatively, a pharmaceutical composition may be in the form of a solution, an aqueous liquid suspension, a non-aqueous liquid suspension, an oil-in-water liquid emulsion, a water-in-oil liquid emulsion, an elixir, or a syrup. Pharmaceutical compositions may or may not be enteric coated. In some embodiments, pharmaceutical compositions are formulated for controlled release, such as delayed or extended release.

In further embodiments, compounds and compositions thereof may be formulated in multi-dose forms, i.e., in the form of multi-particulate dosage forms (e.g., hard gelatin capsules or conventional tablets prepared using a rotary tablet press) comprising one or more bead or minitab populations for oral administration. The conventional tablets rapidly disperse on entry into the stomach. The one or more coated bead or minitab populations may be compressed together with appropriate excipients into tablets (for example, a binder, a diluent/filler, and a disintegrant for conventional tablets.

Tablets, pills, beads, or minitabs of the compounds and compositions of the compounds may be coated or otherwise compounded to provide a dosage form affording the advantage of controlled release, including delayed or extended release, or to protect from the acid conditions of the stomach. For example, the tablet or pill can include an inner dosage and an outer dosage component, the latter being in the form of a coating over the former. The two components can be separated by a polymer layer that controls the release of the inner dosage.

In certain embodiments, the layer may comprise at least one enteric polymer. In further embodiments, the layer may comprise at least one enteric polymer in combination with at least one water-insoluble polymer. In still further embodiments, the layer may comprise at least one enteric polymer in combination with at least one water-soluble polymer. In yet further embodiments, the layer may comprise at least one enteric polymer in combination with a pore-former.

In certain embodiments, the layer may comprise at least one water-insoluble polymer. In still further embodiments, the layer may comprise at least one water-insoluble polymer in combination with at least one water-soluble polymer. In yet further embodiments, the layer may comprise at least one water-insoluble polymer in combination with a pore-former.

Representative examples of water-soluble polymers include polyvinylpyrrolidone (PVP), hydroxypropyl methylcellulose (HPMC), hydroxypropylcellulose (HPC), polyethylene glycol, and the like.

Representative examples of enteric polymers include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methylmethacrylate copolymers and shellac. These polymers may be used as a dry powder or an aqueous dispersion. Some commercially available materials that may be used are methacrylic acid copolymers sold under the trademark Eudragit (LI 00, S I 00, L30D) manufactured by Rohm Pharma, Cellacefate (cellulose acetate phthalate) from Eastman Chemical Co., Aquateric (cellulose acetate phthalate aqueous dispersion) from FMC Corp. and Aqoat (hydroxypropyl methylcellulose acetate succinate aqueous dispersion) from Shin Etsu K.K.

Representative examples of useful water-insoluble polymers include ethylcellulose, polyvinyl acetate (for example, Kollicoat SR #30D from BASF), cellulose acetate, cellulose acetate butyrate, neutral copolymers based on ethyl acrylate and methylmethacrylate, copolymers of acrylic and methacrylic acid esters with quaternary ammonium groups such as Eudragit NE, RS and RS30D, RL or RL30D and the like.

Any of the above polymers may be further plasticized with one or more pharmaceutically acceptable plasticizers. Representative examples of plasticizers include triacetin, tributyl citrate, triethyl citrate, acetyl tri-n-butyl citrate diethyl phthalate, castor oil, dibutyl sebacate, acetylated monoglycerides and the like or mixtures thereof. The plasticizer, when used, may comprise about 3 to 30 wt. % and more typically about 10 to 25 wt. % based on the polymer. The type of plasticizer and its content depends on the polymer or polymers and nature of the coating system (e.g., aqueous or solvent based, solution or dispersion based and the total solids).

Pharmaceutical compositions typically must be sterile and stable under the conditions of manufacture and storage. A composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, a compound can be administered in a time release formulation, for example in a composition which includes a slow release polymer. The compound can be prepared with carriers that will protect against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG).

Pharmaceutical compositions can also include carriers to protect the composition against rapid degradation or elimination from the body, such as a controlled release formulation, including liposomes, hydrogels, and microencapsulated delivery systems. For example, a time delay material such as glyceryl monostearate or glyceryl stearate alone, or in combination with a wax, may be employed. Any drug delivery apparatus may be used to deliver compounds and compositions of the disclosure, including implants (e.g., implantable pumps) and catheter systems, slow injection pumps and devices, all of which are well known to the skilled artisan.

Pharmaceutical compositions may also be in the form of a sterile injectable aqueous or oleagenous (oily) suspension. This suspension may be formulated according to the known art using those suitable dispersing or wetting agents and suspending agents mentioned herein. The sterile injectable preparation may also be a sterile injectable solution or suspension in a non-toxic parenterally acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

Many methods for the preparation of such formulations are generally known to those skilled in the art. Sterile injectable solutions can be prepared by incorporating an active compound, such as a compound of Formula (I) provided herein, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, common methods of preparation are vacuum drying and freeze-drying which yields a powder of the active ingredient plus any additional desired ingredient from a previously sterile-filtered solution thereof. Compounds may also be formulated with one or more additional compounds that enhance their solubility.

It is often advantageous to formulate compositions (such as parenteral compositions) in dosage unit form for ease of administration and uniformity of dosage. The term “unit dosage form” refers to a physically discrete unit suitable as unitary dosages for human subjects and other animals, each unit containing a predetermined quantity of active material calculated to produce the desired therapeutic effect, in association with a suitable pharmaceutical carrier. The specification for the dosage unit forms of the invention may vary and are dictated by and directly dependent on (a) the unique characteristics of the therapeutic compound and the particular therapeutic effect to be achieved, and (b) the limitations inherent in the art of compounding such a therapeutic compound for the prevention or treatment of a KRAS-G12D-associated disease, disorder or condition, such as a cancer or a tumor. Dosages are discussed further below.

In some embodiments, the pharmaceutical composition is provided in a single-use container (e.g., a single-use vial, ampoule, syringe, or autoinjector), whereas a multi-use container (e.g., a multi-use vial) is provided in other embodiments.

Pharmaceutical compositions provided herein can be formulated to be compatible with the intended method or route of administration; exemplary routes of administration are set forth herein. Furthermore, the pharmaceutical compositions may be used in combination with other therapeutically active agents or compounds as described herein in order to treat or prevent the KRAS-G12D-associated diseases, disorders and conditions as contemplated herein.

Pharmaceutical compositions containing the active ingredient (e.g., a KRAS-G12D inhibitor) may be in a form suitable for oral use, for example, as tablets, capsules, troches, lozenges, aqueous or oily suspensions, dispersible powders or granules, emulsions, hard or soft capsules, or syrups, solutions, beads, microbeads or elixirs. Pharmaceutical compositions intended for oral use may be prepared according to any method known in the art for the manufacture of pharmaceutical compositions, and such compositions may contain one or more agents such as, for example, sweetening agents, flavoring agents, coloring agents and preserving agents in order to provide pharmaceutically acceptable preparations. Tablets, capsules and the like generally contain the active ingredient in admixture with non-toxic pharmaceutically acceptable carriers or excipients which are suitable for the manufacture of tablets. These carriers or excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin, gum arabic or acacia, and lubricating agents, for example magnesium stearate, stearic acid or talc.

Tablets, capsules and the like suitable for oral administration may be uncoated or coated using known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil. Aqueous suspensions contain the active materials in admixture with excipients suitable for the manufacture thereof. Such excipients can be suspending agents, for example sodium carboxymethylcellulose, methykellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known in the art.

Pharmaceutical compositions of the present invention may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Pharmaceutical compositions typically comprise a therapeutically effective amount of a KRAS-G12D inhibitor compound provided herein and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bi sulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-MoqJholino)ethanesulfonic acid (MES), 2-(N-Morpholino)ethanesulfonic acid sodium salt (MES), 3-(N-Morpholino)propanesulfonic acid (MOPS), and Ntris[Hydroxyrnethyl]methyl-3-arninopropanesulfonic acid (TAPS). After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form.

In some embodiments, there are provided pharmaceutical compositions that comprise an effective amount of a compound and/or composition described herein, and a pharmaceutically acceptable excipient, carrier or diluent. In an embodiment, there are provided pharmaceutical compositions for the treatment or prevention of a KRAS-G12D-associated disease, disorder or condition, such as a cancer or a tumor, comprising a compound described herein, or a pharmaceutically acceptable salt, ester, hydrate, solvate or stereoisomer thereof, and a pharmaceutically acceptable carrier. In another embodiment, there is provided a pharmaceutical composition for the prevention or treatment of a KRAS-G12D-associated disease, disorder or condition, such as a cancer or a tumor, the composition comprising a compound described herein, or a pharmaceutically acceptable salt thereof, and a pharmaceutically acceptable carrier.

### Methods of Use of Compounds and Compositions

In certain embodiments, there are provided methods for prevention or treatment of a KRAS-G12D-associated disease, disorder or condition in a subject by administering an effective amount of a compound or composition described herein. In a related aspect, there are provided methods for prevention or treatment of a KRAS-G12D-associated hyperplastic or hyperproliferative disorder, e.g., a cancer or a tumor, in a subject in need thereof by administering an effective amount of a compound or composition described herein.

In an embodiment, there is provided herein a method of treating a subject (e.g., a human) with cancer or a disorder mediated by KRAS-G12D comprising the step of administering to the subject a therapeutically effective amount of an KRAS-G12D inhibitor compound provided herein, e.g., a bifunctional compound provided herein or a pharmaceutically acceptable composition thereof.

There is also provided a method of treating a subject (e.g., a human) with cancer or a hyperproliferative disorder mediated by KRAS-G12D comprising the step of administering to the subject a therapeutically effective amount of a compound provided herein, e.g., a compound provided herein or a pharmaceutically acceptable composition thereof. In certain embodiments, the amount of a compound in a composition is such that it is effective as an inhibitor of KRAS-G12D in a biological sample (e.g., in a cellular assay, in an in vivo model, etc.) or in a subject. In certain embodiments, the composition is formulated for administration to a subject in need of such composition. In some embodiments, the composition is an injectable formulation. In other embodiments, the composition is formulated for oral administration to a subject. In some embodiments, the composition is in the form of a hard shell gelatin capsule, a soft shell gelatin capsule, a cachet, a pill, a tablet, a lozenge, a powder, a granule, a pellet, a pastille, or a dragee. In some embodiments, the composition is in the form of a solution, an aqueous liquid suspension, a non-aqueous liquid suspension, an oil-in-water liquid emulsion, a water-in-oil liquid emulsion, an elixir, or a syrup. In some embodiments, the composition is enteric coated. In some embodiments, the composition is formulated for controlled release.

In further embodiments, there are provided methods for treating or preventing cancer in a subject, comprising administering to the subject a therapeutically effective amount of at least one compound of the disclosure and at least one additional signal transduction inhibitor (STI). In a particular embodiment, the at least one STI is selected from the group consisting of bcr/abl kinase inhibitors, epidermal growth factor (EGF) receptor inhibitors, her-2/neu receptor inhibitors, and farnesyl transferase inhibitors (FTIs). There are also provided methods of augmenting the rejection of tumor cells in a subject comprising administering a compound of the disclosure in conjunction with at least one chemotherapeutic agent and/or radiation therapy, wherein the resulting rejection of tumor cells is greater than that obtained by administering either the compound, the chemotherapeutic agent or the radiation therapy alone. In further embodiments, there are provided methods for treating cancer in a subject, comprising administering to the subject a therapeutically effective amount of at least one compound of the disclosure and at least one immunomodulator.

In further embodiments, there are provided methods for treating, inhibiting or preventing a hyperproliferative or hyperplastic disease or disorder in a subject, comprising administering to the subject an effective amount of at least one compound or pharmaceutical composition of the disclosure.

The terms “patient” and “subject” are used interchangeably herein to refer to a human or a non-human animal (e.g., a mammal). Non-limiting examples of subjects include humans, monkeys, cows, rabbits, sheep, goats, pigs, dogs, cats, rats, mice, and transgenic species thereof. In some embodiments, a subject is in need of treatment by the methods provided herein, and is selected for treatment based on this need. A subject in need of treatment is art-recognized, and includes subjects that have been identified as having a disease or condition (e.g., cancer, tumor, hyperproliferative disorder), or having a symptom of such a disease or condition, or being at risk of such a disease or condition, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder). In some embodiments, a subject has a cancer or tumor carrying the KRAS-G12D mutation.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “administration”, “administer” and the like, as they apply to, for example, a subject, cell, tissue, organ, or biological fluid, refer to contact of, for example, an inhibitor of KRAS-G12D, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, “treatment” and the like refer to a course of action (such as administering an inhibitor of KRAS-12 or a pharmaceutical composition comprising same) initiated after a disease, disorder or condition, or a symptom thereof, has been diagnosed, observed, and the like, so as to eliminate, alleviate, reduce, suppress, mitigate, improve, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease. Specifically, the term “treatment”, as used in the present application, means that a therapeutic substance including a compound or composition according to the present disclosure is administered to a patient in need thereof. In certain embodiments, the term “treatment” also relates to the use of a compound or composition according to the present disclosure, optionally in combination with one or more anticancer agents, to alleviate one or more symptoms associated with KRAS-G12D, to slow down the development of one or more symptoms related to KRAS-G12D, to reduce the severity of one or more symptoms related to KRAS-G12D, to inhibit the clinical manifestations related to KRAS-G12D mutation, and/or to inhibit the expression of adverse symptoms associated with the KRAS-G12D mutation.

The terms “prevent”, “preventing”, “prevention” and the like refer to a course of action (such as administering a KRAS-G12D inhibitor or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof: generally in the context of a subject predisposed to having a particular disease, disorder or condition. In certain instances, the terms also refer to slowing the progression of the disease, disorder or condition or inhibiting progression thereof to a harmful or otherwise undesired state. Specifically, the term “prevention”, as used in the present application, means that a therapeutic substance including a compound or composition according to the present disclosure is administered to a subject to prevent the occurrence of diseases related to the KRAS-G12D mutation.

The term “in need of prevention” as used herein refers to a judgment made by a physician or other caregiver that a subject requires or will benefit from preventative care. This judgment is made based on a variety of factors that are in the realm of a physician's or caregiver's expertise.

The terms “therapeutically effective amount” and “effective amount” are used interchangeably herein to refer to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of a KRAS-G12D inhibitor (or, e.g., a metabolite thereof) at a particular time post-administration may be indicative of whether a therapeutically effective amount has been used. In some embodiments, the terms “therapeutically effective amount” and “effective amount” refer to the amount or dose of a therapeutic agent, such as a compound, upon single or multiple dose administration to a subject, which provides the desired therapeutic, diagnostic, or prognostic effect in the subject. An effective amount can be readily determined by an attending physician or diagnostician using known techniques and by observing results obtained under analogous circumstances. In determining the effective amount or dose of compound administered, a number of factors are considered including, but not limited to: the size, age, and general health of the subject; the specific disease involved; the degree of or involvement or the severity of the disease or condition to be treated; the response of the individual subject; the particular compound administered; the mode of administration; the bioavailability characteristics of the preparation administered; the dose regimen selected; the use of concomitant medication(s); and other relevant considerations.

The term “substantially pure” is used herein to indicate that a component makes up greater than about 50% of the total content of the composition, and typically greater than about 60% of the total content. More typically, “substantially pure” refers to compositions in which at least 75%, at least 85%), at least 90% or more of the total composition is the component of interest. In some cases, the component of interest will make up greater than about 90%), or greater than about 95%) of the total content of the composition.

As used herein, the terms “KRAS-G12D-associated disease, disorder or condition” and “disease, disorder or condition mediated by KRAS-G12D” are used interchangeably to refer to any disease, disorder or condition for which the KRAS-G12D mutation is known to play a role, and/or for which treatment with a KRAS-G12D inhibitor may be beneficial. In general, KRAS-G12D-associated or mediated diseases, disorders and conditions are those in which KRAS activity plays a biological, mechanistic, or pathological role. Non-limiting examples of KRAS-G12D-associated diseases, disorders and conditions include oncology-related disorders (cancers, tumors, etc.), including hyperproliferative disorders, hyperplastic diseases, and malignant tumors, such as lung cancer, non-small cell lung cancer (NSCLC), pancreatic cancer, colorectal cancer, colon cancer, cholangiocarcinoma, cervical cancer, bladder cancer, liver cancer or breast cancer. For example, a KRAS-G12D inhibitor (i.e., a compound or composition of the disclosure) may be used to prevent or treat a proliferative condition, cancer or tumor.

In some embodiments, a KRAS-G12D inhibitor is used to prevent or treat one or more of non-small cell lung cancer, pancreatic cancer, colorectal cancer, bile duct cancer, cervical cancer, bladder cancer, liver cancer and breast cancer.

KRAS-G12D inhibitor compounds and compositions provided herein may be administered to a subject in any appropriate manner known in the art. Suitable routes of administration include, without limitation: oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), extra-gastrointestinal, nasal, vaginal, sublingual, intraocular, rectal, topical (e.g., transdermal), buccal and inhalation. Depot injections, which are generally administered subcutaneously or intramuscularly, may also be utilized to release the KRAS-G12D inhibitors disclosed herein over a defined period of time. In certain embodiments, KRAS-G12D inhibitor compounds and compositions are administered orally to a subject in need thereof.

KRAS-G12D inhibitor compounds and compositions provided herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan. In general, dosing parameters dictate that the dosage amount be less than an amount that could be irreversibly toxic to the subject (the maximum tolerated dose (MID)) and not less than an amount required to produce a measurable effect on the subject. Such amounts are determined by, for example, the pharmacokinetic and pharmacodynamic parameters associated with ADME, taking into consideration the route of administration and other factors.

In some embodiments, an KRAS-G12D inhibitor may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect. For administration of an oral agent, the compositions can be provided in the form of tablets, capsules and the like containing from 1.0 to 1000 milligrams of the active ingredient, particularly 1, 3, 5, 10, 15, 20, 25, 50, 75, 100, 150, 200, 250, 300, 400, 500, 600, 750, 800, 900, or 1000 milligrams of the active ingredient.

In some embodiments, the dosage of the desired KRAS-G12D inhibitor is contained in a “unit dosage form”. The phrase “unit dosage form” refers to physically discrete units, each unit containing a predetermined amount of the KRAS-G12D inhibitor, either alone or in combination with one or more additional agents, sufficient to produce the desired effect. It will be appreciated that the parameters of a unit dosage form will depend on the particular agent(s) and the effect to be achieved.

### Kits

There are also provided herein kits comprising a KRAS-G12D inhibitor compound or composition of the disclosure. Kits are generally in the form of a physical structure housing various components and may be used, for example, in practicing the methods provided herein. For example, a kit may include one or more KRAS-G12D inhibitor disclosed herein (provided in, e.g., a sterile container), which may be in the form of a pharmaceutical composition suitable for administration to a subject. The KRAS-G12D inhibitor can be provided in a form that is ready for use (e.g., a tablet or capsule) or in a form requiring, for example, reconstitution or dilution (e.g., a powder) prior to administration. When the KRAS-G12D inhibitors are in a form that needs to be reconstituted or diluted by a user, the kit may also include diluents (e.g., sterile water), buffers, pharmaceutically acceptable excipients, and the like, packaged with or separately from the KRAS-G12D inhibitors. When combination therapy is contemplated, the kit may contain several therapeutic agents separately or they may already be combined in the kit. Each component of the kit may be enclosed within an individual container, and all of the various containers may be within a single package. A kit of the present invention may be designed for conditions necessary to properly maintain the components housed therein (e.g., refrigeration or freezing).

A kit may also contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention. Unless otherwise stated, the materials and instruments used in this invention are commercially available.

### Compound Synthesis

Compounds provided herein can be synthesized stepwise (step by step) or using a modular method. Scheme A shows the synthetic method for preparing exemplary intermediate compounds. Scheme B shows the synthetic steps used to prepare exemplary compounds. Compounds of the disclosure are synthesized using the appropriate intermediates and raw materials according to the target compounds.

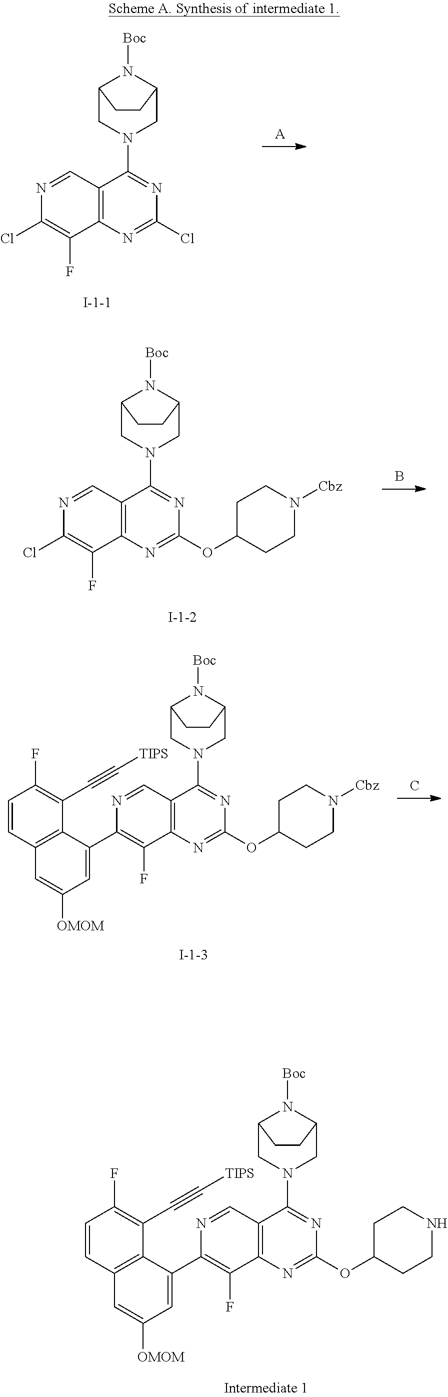

Synthesis of intermediate 1. Step A: To a solution of Compound I-1-1 (0.9 g, 2.10 mmol, 1 eq) in 10 mL of DMSO/Dioxane (1/5) was added benzyl 4-hydroxypiperidine-1-carboxylate (988.82 mg, 4.20 mmol, 2 eq) and Cs₂CO₃ (2.05 g, 6.3 mmol, 3 eq). The mixture was heated to 90° C. and stirred for 12 h, then cooled to room temperature. The mixture was treated with NH₄Cl aqueous and EtOAc, and stirred for 5 min, and the organic layer was then separated. The organic layer was washed with brine, dried over Na₂SO₄ and filtered. The filtrate was concentrated and the residue was purified by column chromatography to afford yellow solid I-1-2 (760 mg, 67% yield) ¹H NMR (500 MHz, CDCl₃) δ 8.73 (s, 1H), 7.39-7.30 (m, 5H), 5.33 (q, J=4.1 Hz, 1H), 5.15 (s, 2H), 4.48-4.27 (m, 4H), 3.91 (s, 2H), 3.65 (s, 2H), 3.40 (ddd, J=13.0, 8.5, 3.6 Hz, 2H), 2.00 (d, J=39.5 Hz, 4H), 1.85 (s, 2H), 1.70 (d, J=7.7 Hz, 2H), 1.51 (s, 9H). m/z (ESI): 627 [M+H]⁺.

Step B: To a solution of I-1-2 (1 g, 1.59 mmol, 1 eq) in 10 mL of THF/Water (10/3) was added K₃PO₄ (1.01 g, 4.78 mmol, 3 eq), cataCXium A Pd-G3 (174.13 mg, 239.19 μmol, 0.15 eq) and ((2-fluoro-6-(methoxymethoxy)-8-(4,4,5,5-tetramethyl-1,3,2-dioxaborolan-2-yl)naphthalen-1-yl)ethynyl)triisopropylsilane (1.14 g, 2.23 mmol, 1.4 eq). The mixture was heated to 80° C. and stirred for 4 h under nitrogen atmosphere, then cooled to room temperature. The mixture was treated with EtOAc and water and stirred for 10 min, and the organic layer was then separated. The organic layer was washed with brine, dried over Na₂SO₄ and filtered. The filtrate was concentrated and the residue was purified by column chromatography (EA/PE=1/1) to afford yellow solid a-3 (Ig, 64% yield). m/z (ESI): 977.6 [M+H]⁺.

Step C: Pd(OH)₂ (50 mg, 1.02 mmol, 1 eq) was added to a solution of I-1-3 (1 g, 1.02 mmol, 1 eq) in 10 mL of THF. The mixture was stirred at room temperature for 12 h under hydrogen atmosphere, then filtered. The filtrate was concentrated to afford yellow solid Intermediate 1 (0.8 g, 90% yield). m/z (ESI): 843.4 [M+H]⁺.

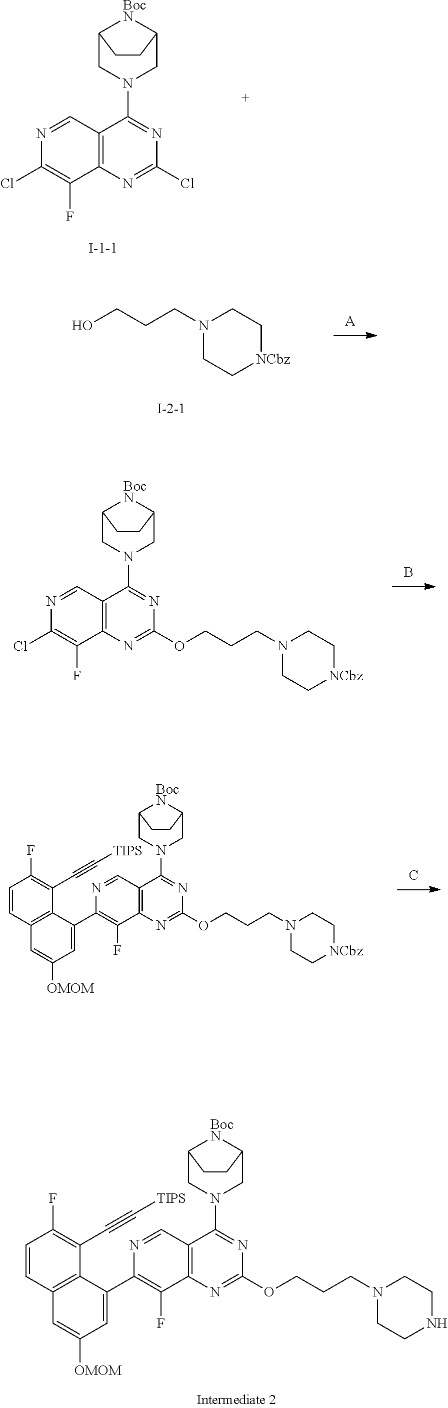

### Synthesis of Intermediate 2

Synthesis of intermediate 2. Intermediate 2 was synthesized according to the procedure of intermediate 1 with I-2-1 as starting material. m/z (ESI): 887.0 [M+H]⁺.

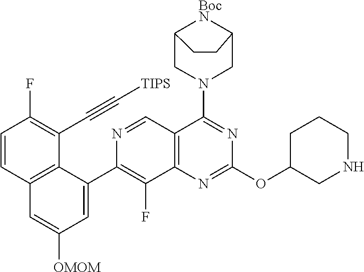

### Intermediate 3

Synthesis of intermediate 3. Intermediate 3 was synthesized according to the procedure of intermediate 1 with benzyl 3-hydroxypiperidine-1-carboxylate as starting material. m/z (ESI): 843.4 [M+H]⁺.

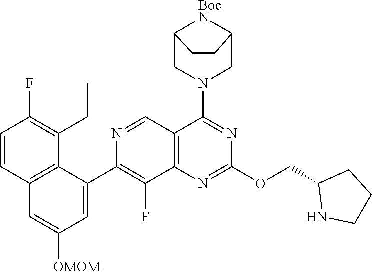

### Intermediate 4

Synthesis of intermediate 4. Intermediate 4 was synthesized according to the procedure of intermediate 1 with Cbz-L-prolinol as starting material. m/z (ESI): 691.6 [M+H]⁺.

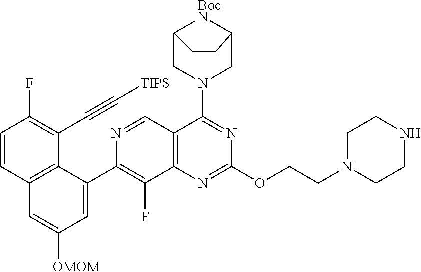

### Intermediate 5

Synthesis of intermediate 5. Intermediate e was synthesized according to the procedure of intermediate 1 with benzyl 4-(2-hydroxyethyl)piperazine-1-carboxylate as starting material. m/z (ESI): 872.5 [M+H]⁺.

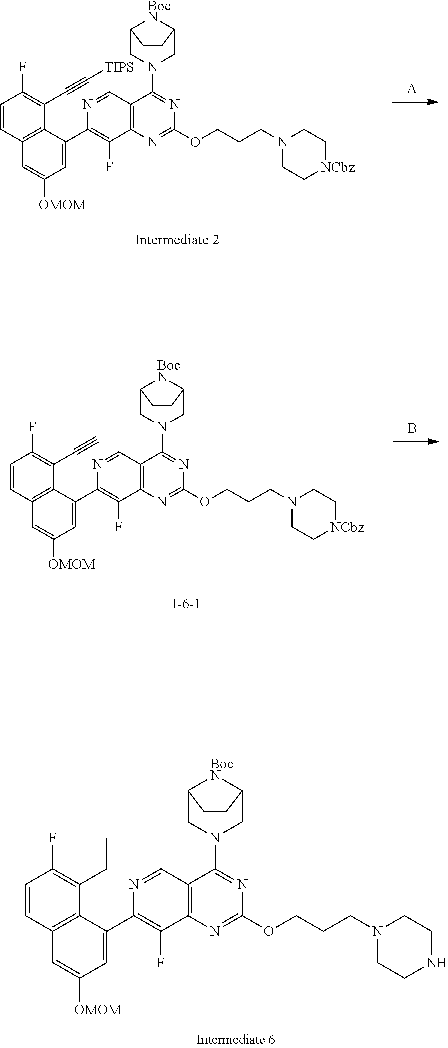

### Intermediate 6

Synthesis of intermediate 6. Step A: TBAF (27.23 mg, 104.14 μmol, 3 eq) was added to a solution of intermediate 2 (50 mg, 52.07 μmol, 1 eq) in 2 mL of THF. The mixture was stirred at room temperature for 10 min, then concentrated to afford I-6-1 that was used in the next step directly. m/z (ESI): 865 [M+H]⁺.

Step B: Intermediate 6 was synthesized according to the Step C in the procedure of intermediate 1. m/z (ESI): 735 [M+H]⁺.

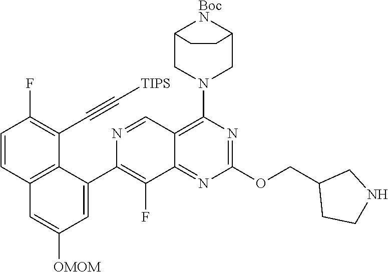

### Intermediate 7

Synthesis of intermediate 7. Intermediate 7 was synthesized according to the procedure of intermediate 1 with benzyl 3-(hydroxymethyl)pyrrolidine-1-carboxylate as starting material. m/z (ESI): 843.3 [M+H]⁺.

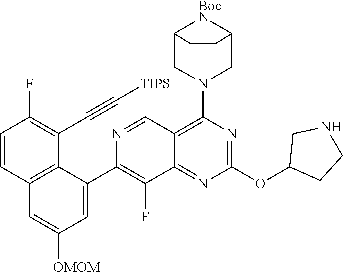

### Intermediate 8

Synthesis of intermediate 8. Intermediate 8 was synthesized according to the procedure of intermediate 1 with benzyl 3-hydroxypyrrolidine-1-carboxylate as starting material. m/z (ESI): 830 [M+H]⁺.

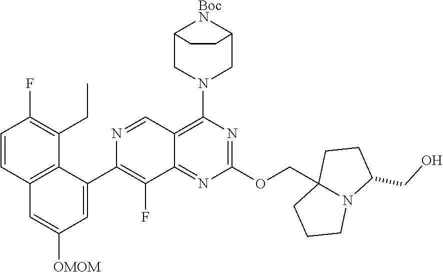

### Intermediate 9

Synthesis of intermediate 9. Intermediate 9 was synthesized according to the procedure of intermediate 6 with ((3R)-3-(((tert-butyldimethylsilyl)oxy)methyl)tetrahydro-1H-pyrrolizin-7a(5H)-yl)methanol as starting material. m/z (ESI): 761.5 [M+H]⁺.

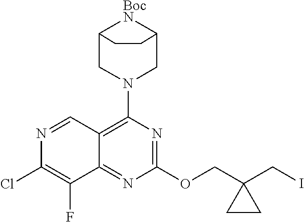 [SMILES: CC(C)(C)OC(=O)N1C2CCC1CN(c1nc(OCC3(CI)CC3)nc3c(F)c(Cl)ncc13)C2]

### Intermediate 10

Synthesis of intermediate 10. Intermediate 10 was synthesized according to the procedure of intermediate 1.

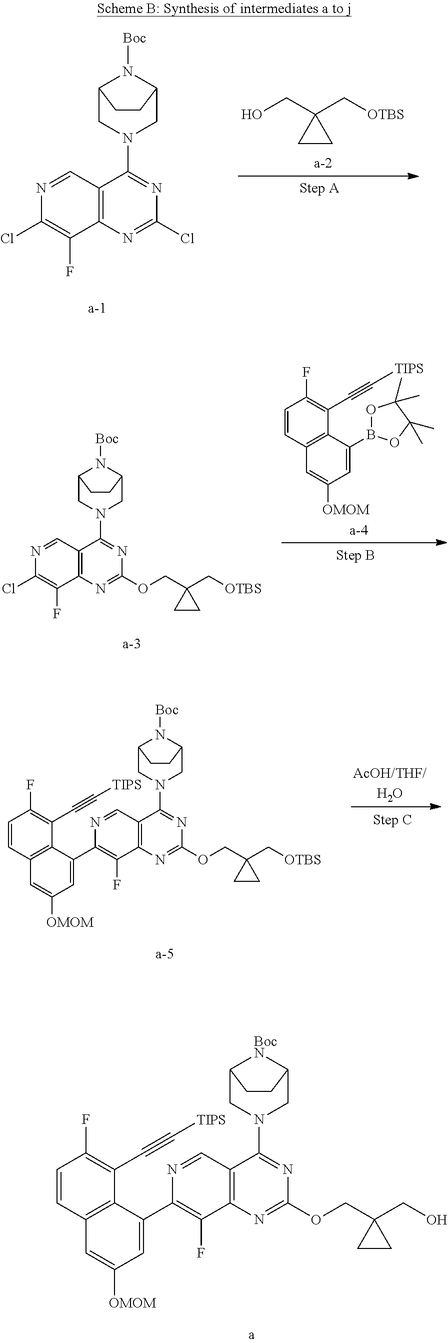

### Synthesis of Intermediate a

Synthesis of intermediate a. Step A: A solution of compound a-2 (3.03 g, 14.01 mmol, 1.2 eq) in anhydrous THF (30 mL) was cooled to 0° C., followed by addition of 60% NaH (607.2 mg, 15.18 mmol, 1.3 eq). The mixture was warmed to 25° C. slowly and stirred for 30 min, then cooled to 0° C., followed by addition of compound a-1 (5 g, 11.67 mmol, 1 eq). The mixture was warmed to 25° C. and stirred for 4 h, then treated with ethyl acetate and water. The mixture was stirred for 10 min and the organic layer was separated. The organic layer was washed with water and brine, then dried over Na₂SO₄ and filtered. The filtrate was concentrated under reduced pressure and the residue was purified by column chromatography (PE:EA=10:1) to afford compound a-3 (4.7 g, yield 66.19%).

Step B: Compound a-4 (3.41 g, 6.66 mmol, 1.5 eq) was added to a solution of compound a-3 (2.7 g, 4.44 mmol, 1 eq) in dioxane (15 mL), followed by addition of K₃PO₄ (2.82 g, 13.32 mmol, 3 eq) and cataCXium A Pd G3 (646.4 mg, 888 μmol, 0.2 eq). The mixture was warmed to 90° C. under the nitrogen atmosphere and stirred for 2 h, then cooled to 25° C. The mixture was treated with ethyl acetate and water. The mixture was stirred for 10 min and the organic layer was separated. The organic layer was washed with water and brine, then dried over Na₂SO₄ and filtered. The filtrate was concentrated under reduced pressure and the residue was purified by column chromatography (PE:EA=10:1) to afford compound a-5 (3.3 g, yield 77.57%).

Step C: A solution of AcOH (9 mL) in water (3 mL) was added to another solution of compound a-5 (3.3 g, 3.44 mmol, 1 eq) in THF (3 mL). The mixture was stirred at 25° C. for 24 h, and the pH was adjusted to 7 with NaHCO₃ aqueous solution. The mixture was extracted with ethyl acetate, the combined organic layer was washed with water and brine, then dried over Na₂SO₄ and filtered. The filtrate was concentrated under reduced pressure and the residue was purified by column chromatography (PE:EA=10:3) to afford intermediate a (2.5 g, yield 86.01%). m/z (ESI): 845.03 [M+H]⁺.

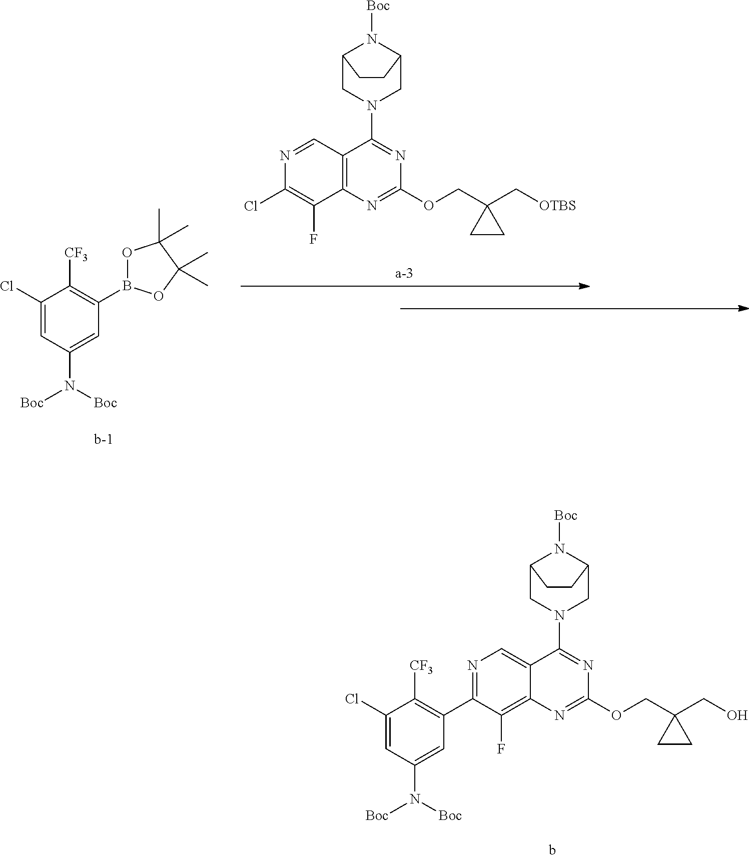 [SMILES: CC(C)(C)OC(=O)N(C(=O)OC(C)(C)C)c1cc(Cl)c(C(F)(F)F)c(B2OC(C)(C)C(C)(C)O2)c1 ; CC(C)(C)OC(=O)N1C2CCC1CN(c1nc(OCC3(CO[Si](C)(C)C(C)(C)C)CC3)nc3c(F)c(Cl)ncc13)C2 ; CC(C)(C)OC(=O)N(C(=O)OC(C)(C)C)c1cc(Cl)c(C(F)(F)F)c(-c2ncc3c(N4CC5CCC(C4)N5C(=O)OC(C)(C)C)nc(OCC4(CO)CC4)nc3c2F)c1]

### Intermediate b

Synthesis of intermediate b. Intermediate b was synthesized according to the procedure of intermediate a with compound b-1 as start material. m/z (ESI): 853.1 [M+H]⁺.

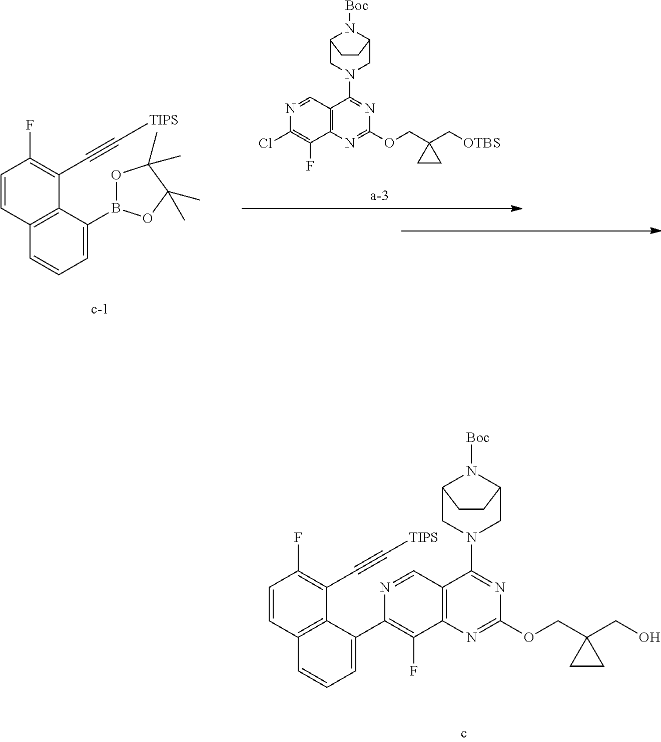

### Intermediate c

Synthesis of intermediate e. Intermediate c was synthesized according to the procedure of intermediate a with compound c-1 as start material.

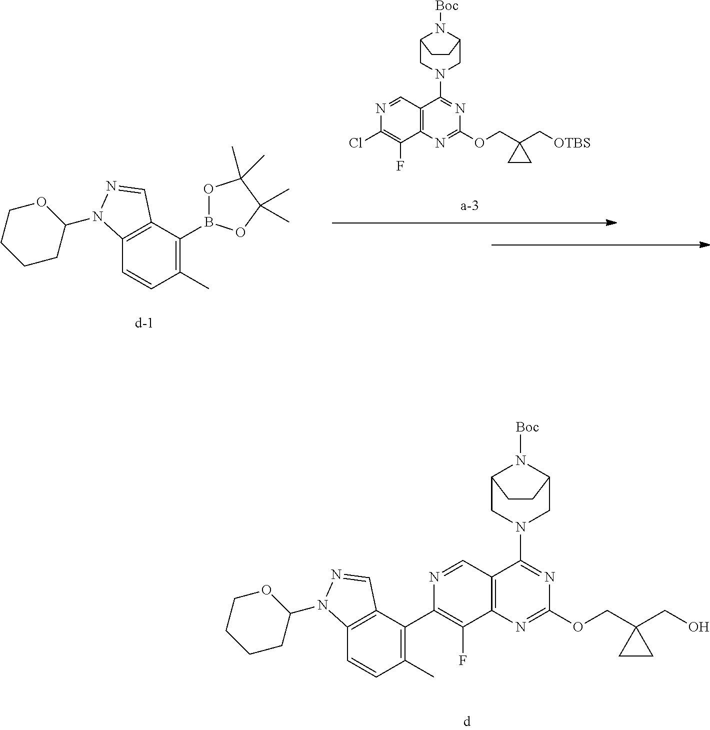 [SMILES: Cc1ccc2c(cnn2C2CCCCO2)c1B1OC(C)(C)C(C)(C)O1 ; CC(C)(C)OC(=O)N1C2CCC1CN(c1nc(OCC3(CO[Si](C)(C)C(C)(C)C)CC3)nc3c(F)c(Cl)ncc13)C2 ; Cc1ccc2c(cnn2C2CCCCO2)c1-c1ncc2c(N3CC4CCC(C3)N4C(=O)OC(C)(C)C)nc(OCC3(CO)CC3)nc2c1F]

### Intermediate d

Synthesis of intermediate d. Intermediate d was synthesized according to the procedure of intermediate a with compound d-1 as start material. ¹H NMR (400 MHz, Chloroform-d) δ 9.18 (s, 1H), 7.79 (s, 1H), 7.67 (d, J=8.6 Hz, 1H), 7.41 (d, J=8.6 Hz, 1H), 5.80 (dd, J=8.9, 2.7 Hz, 1H), 4.51 (d, J=31.0 Hz, 6H), 4.08 (d, J=11.6 Hz, 1H), 3.81 (dt, J=12.3, 5.9 Hz, 3H), 3.50 (s, 2H), 2.60 (d, J=11.5 Hz, 1H), 2.47 (d, J=7.6 Hz, 1H), 2.42 (s, 3H), 2.20 (s, 1H), 2.10 (s, 2H), 2.05 (s, 2H), 1.82-1.77 (m, 3H), 1.57 (s, 10H), 0.73 (d, J=4.8 Hz, 2H), 0.66 (s, 2H).

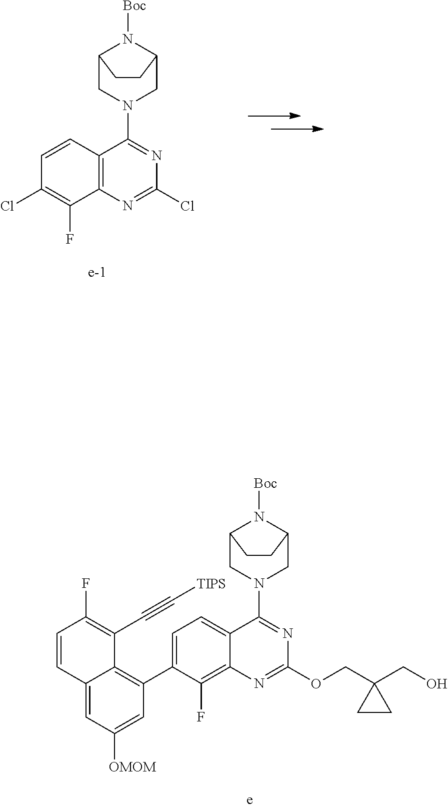

### Intermediate e

Synthesis of intermediate e. Intermediate e was synthesized according to the procedure of intermediate a with compound e-1 as start material.

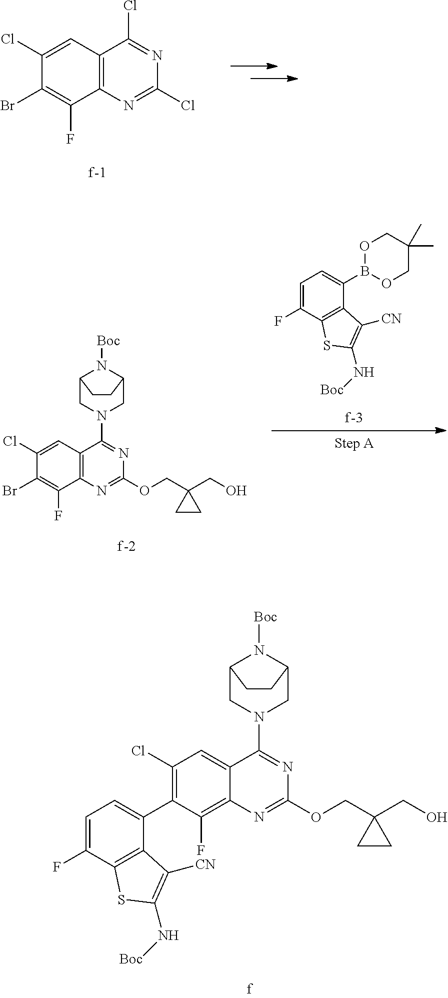 [SMILES: Fc1c(Br)c(Cl)cc2c(Cl)nc(Cl)nc12 ; CC(C)(C)OC(=O)N1C2CCC1CN(c1nc(OCC3(CO)CC3)nc3c(F)c(Br)c(Cl)cc13)C2 ; CC1(C)COB(c2ccc(F)c3sc(NC(=O)OC(C)(C)C)c(C#N)c23)OC1 ; CC(C)(C)OC(=O)Nc1sc2c(F)ccc(-c3c(Cl)cc4c(N5CC6CCC(C5)N6C(=O)OC(C)(C)C)nc(OCC5(CO)CC5)nc4c3F)c2c1C#N]

### Intermediate f

Synthesis of intermediate f Compound f-2 was synthesized according to the procedure of compound a-3 with compound f-1 as start material. m/z (ESI): 571.4 [M+H]⁺.

Step A: Compound f-3 (150 mg, 371.05 μmol, 1 eq) was added to a solution of compound f-2 (190.97 mg, 333.94 μmol, 0.9 eq) in dioxane (1 mL), followed by addition of TMSOK (6.33 mg, 742.09 μmol, 2 eq) and DPEPhosPdCl2 (26.53 mg, 37.10 μmol, 0.1 eq). The mixture was warmed to 80° C. under nitrogen atmosphere and stirred for 2 h, then cooled to 25° C. The mixture was concentrated under reduced pressure and the residue was purified by column chromatography (EA/PE=1/2) to afford intermediate f (120 mg, yield 41.29%). m/z (ESI): 783.7 [M+H]⁺.

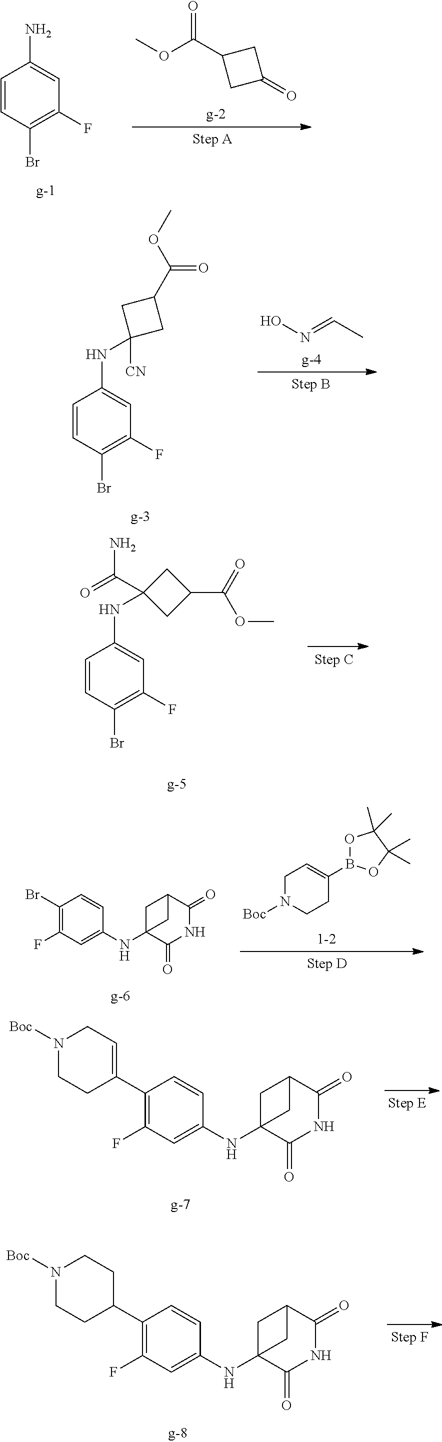

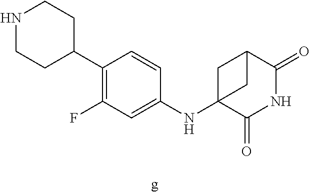 [SMILES: O=C1NC(=O)C2(Nc3ccc(C4CCNCC4)c(F)c3)CC1C2]

### Intermediate g

Synthesis of intermediate g. Step A: Compound g-2 (2 g, 15.61 mmol, 1 eq) was added to a solution of compound g-1 (3.26 g, 17.17 mmol, 1.1 eq), followed by addition of trimethylsilyl cyanide (3.10 g, 31.22 mmol, 2 eq). The mixture was stirred at 25° C. for 16 h, then concentrated under reduced pressure and the residue was purified by column chromatography (MeOH/DCM=0%-3%) to afford compound g-3 (2.3 g, yield 45.1%).

Step B: Compound g-4 (1.25 g, 21.09 mmol, 3 eq) was added to a solution of compound g-3 (2.3 g, 7.03 mmol, 1 eq) in toluene (15 mL), followed by addition of InCl₃ (31.10 mg, 140.61 μmol, 0.02 eq). The mixture was warmed to 110° C. and stirred for 3 h, then cooled to 25° C. The mixture was quenched with water and extracted with ethyl acetate. The combined organic layer was washed with water and brine, then dried over Na₂SO₄ and filtered. The filtrate was concentrated under reduced pressure and the residue was purified by column chromatography (MeOH/DCM=0%-4%) to afford compound g-5 (1.7 g, yield 70.1%).

Step C: t-BuOK (455.13 mg, 4.06 mmol, 2 eq) was added to a solution of compound g-5 (700 mg, 2.03 mmol, 1 eq) in THF (5 mL). The mixture was stirred at 25° C. for 3 h, then quenched with water, and extracted with ethyl acetate. The combined organic layer was washed with water and brine, then dried over Na₂SO₄ and filtered. The filtrate was concentrated under reduced pressure and the residue was purified by column chromatography (MeOH/DCM=0%-3%) to afford compound g-6 (341 mg, yield 53.5%).

Step D: Compound 1-2 (671.50 mg, 2.17 mmol, 2 eq) was added to a solution of compound g-6 (340 mg, 1.09 mmol, 1 eq) in dioxane (5 mL) and water (0.5 mL), followed by addition of K₃PO₄ (691.47 mg, 3.26 mmol, 3 eq) and cataCXium A Pd G3 (157.94 mg, 217.17 μmol, 0.2 eq). The mixture was warmed to 100° C. under the nitrogen atmosphere and stirred for 3 h, then cooled to 25° C. The reaction was quenched with water and extracted with ethyl acetate. The combined organic layer was washed with water and brine, then dried over Na₂SO₄ and filtered. The filtrate was concentrated under reduced pressure and the residue was purified by column chromatography (MeOH/DCM=0%-4%) to afford compound g-7 (430 mg, yield 95.3%).

Step E: 10% Pd/C (0.23 g) was added to a solution of compound g-7 (430 mg, 1.04 mmol, 1 eq) in THF (10 mL). The mixture was stirred at 25° C. under hydrogen atmosphere for 15 h, then filtered. The filtrate was concentrated under reduced pressure to afford compound g-8 (320 mg, yield 74.1%).

Step F: 4M HCl in dioxane (2 mL) was added to a solution of compound g-8 (320 mg, 766.52 μmol, 1 eq) in DCM (5 mL). The mixture was stirred at 25° C. for 30 min, then concentrated under reduced pressure to afford intermediate g (300 mg, yield 98.3%). m/z (ESI): 318.4 [M+H]⁺.

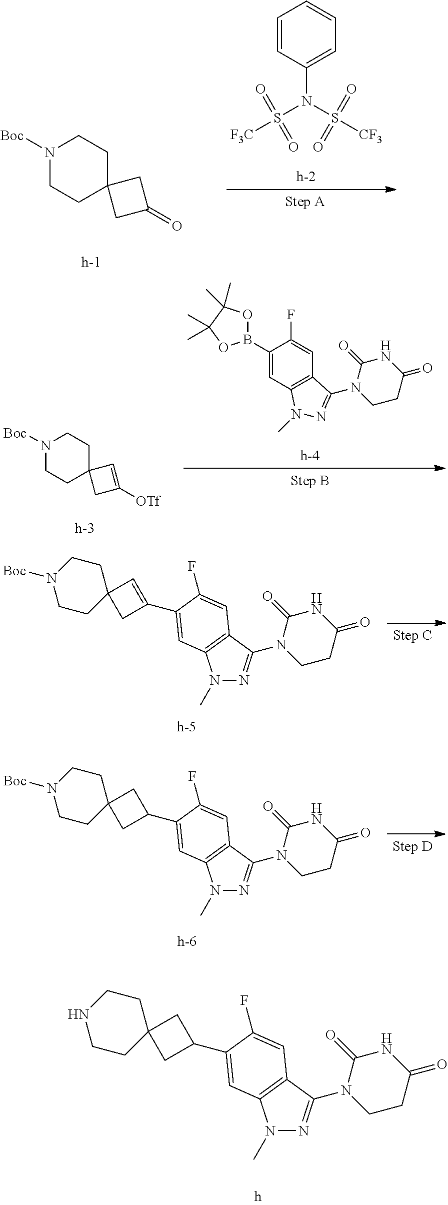

### Synthesis of Intermediate h

Synthesis of intermediate h. Step A: A solution of compound h-1 (0.5 g, 2.09 mmol, 1 eq) in THF (10 mL) was cooled to 0° C. under nitrogen atmosphere, followed by addition of LDA (2 M, 2.09 mL, 2 eq). The mixture was stirred at 0° C. for 30 min, then treated with compound h-2 (895.70 mg, 2.51 mmol, 1.2 eq). The mixture was warmed to 25° C. and stirred for 1 h, then quenched with water. The mixture was extracted with DCM. The combined organic layer was washed with water and brine, then dried over Na₂SO₄ and filtered. The filtrate was concentrated under reduced pressure and the residue was purified by column chromatography (MeOH/DCM=0%-1%) to afford compound h-3 (400 mg, yield 51.6%).

Step B: Compound h-4 (200.70 mg, 517.00 μmol, 0.6 eq) was added to a solution of compound h-3 (320 mg, 861.67 μmol, 1 eq) in THF (5 mL) and water (0.5 mL), followed by addition of K₃PO₄ (548.72 mg, 2.59 mmol, 3 eq) and cataCXium A Pd G3 (46.92 mg, 172.33 μmol, 0.2 eq). The mixture was warmed to 60° C. under the nitrogen atmosphere and stirred for 3 h, then cooled to 25° C. The mixture was quenched with water and extracted with DCM. The combined organic layer was washed with water and brine, then dried over Na₂SO₄ and filtered. The filtrate was concentrated under reduced pressure and the residue was purified by column chromatography (MeOH/DCM=0%-3%) to afford compound h-5 (190 mg, yield 45.6%).

Step C: 10% Pd/C (100 mg) was added to a solution of compound h-5 (190 mg, 392.94 μmol, 1 eq) in THF (10 mL) and MeOH (5 mL). The mixture was stirred at 40° C. under hydrogen atmosphere for 15 h, then cooled to 25° C. and filtered. The filtrate was concentrated under reduced pressure to afford compound h-6 (140 mg, yield 73.4%).

Step D: 4M HCl in dioxane (1.5 mL) was added to a solution of compound h-6 (140 mg, 288.33 μmol, 1 eq) in MeOH (3 mL). The mixture was stirred at 25° C. for 30 min, and pH was adjusted to 5-6 with NaHCO₃ aqueous solution. The mixture was extracted with EA, and the combined organic layer was washed with water and brine, then dried over Na₂SO₄ and filtered. The filtrate was concentrated under reduced pressure and the residue was purified by column chromatography (MeOH/DCM=0%-4%) to afford intermediate h (100 mg, yield 89.9%). m/z (ESI): 386.4 [M+H]+.

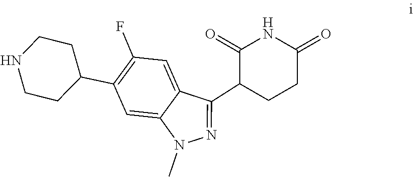 [SMILES: Cn1nc(C2CCC(=O)NC2=O)c2cc(F)c(C3CCNCC3)cc21]

### Intermediate i

Synthesis of intermediate i. Intermediate i was synthesized according to the procedure of intermediate h.

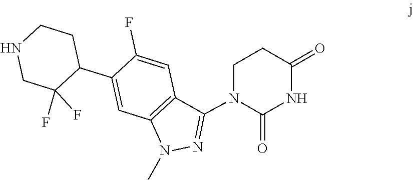 [SMILES: Cn1nc(N2CCC(=O)NC2=O)c2cc(F)c(C3CCNCC3(F)F)cc21]

### Intermediate j

Synthesis of intermediate j. Intermediate j was synthesized according to the procedure of intermediate h. m/z (ESI): 382.0 [M+H]+.

### Example 1. Synthesis of Compound 130 Salt

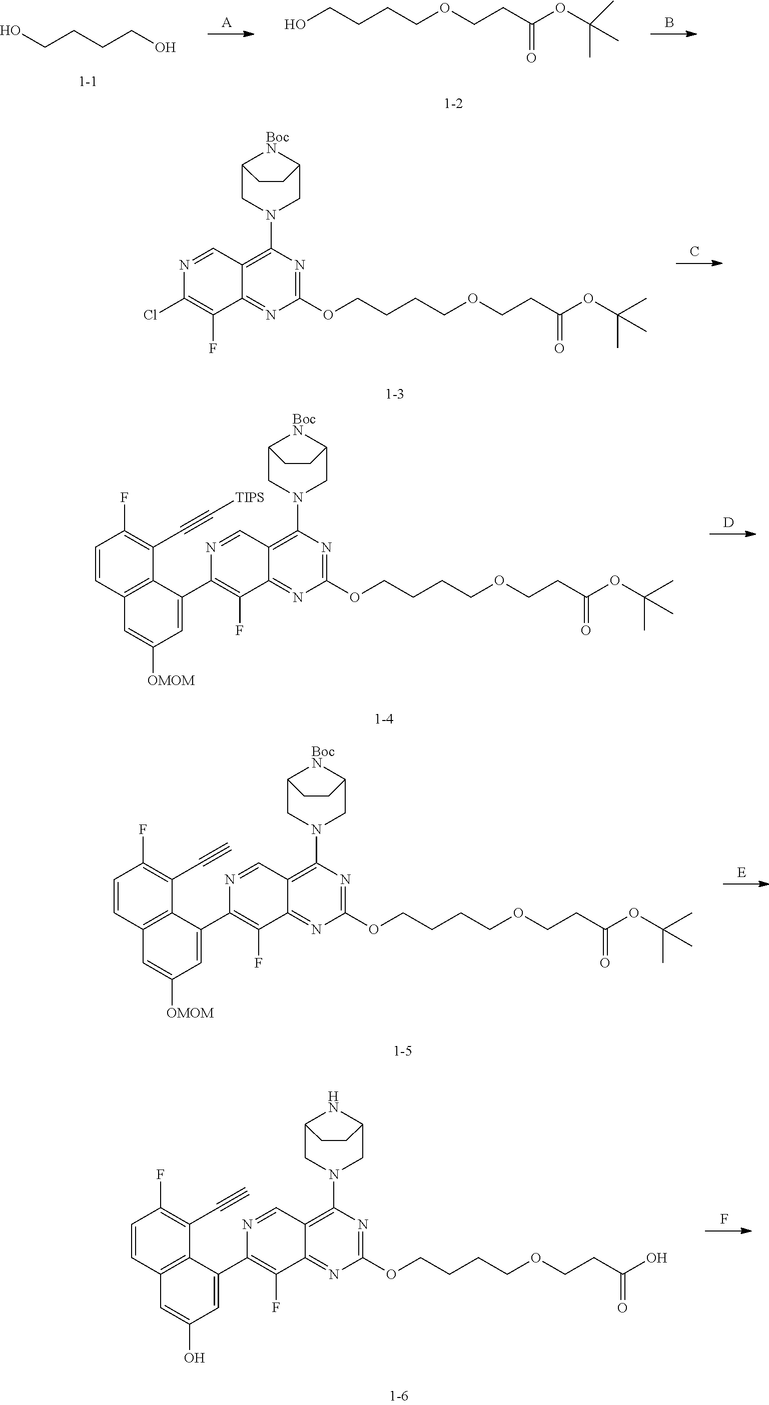

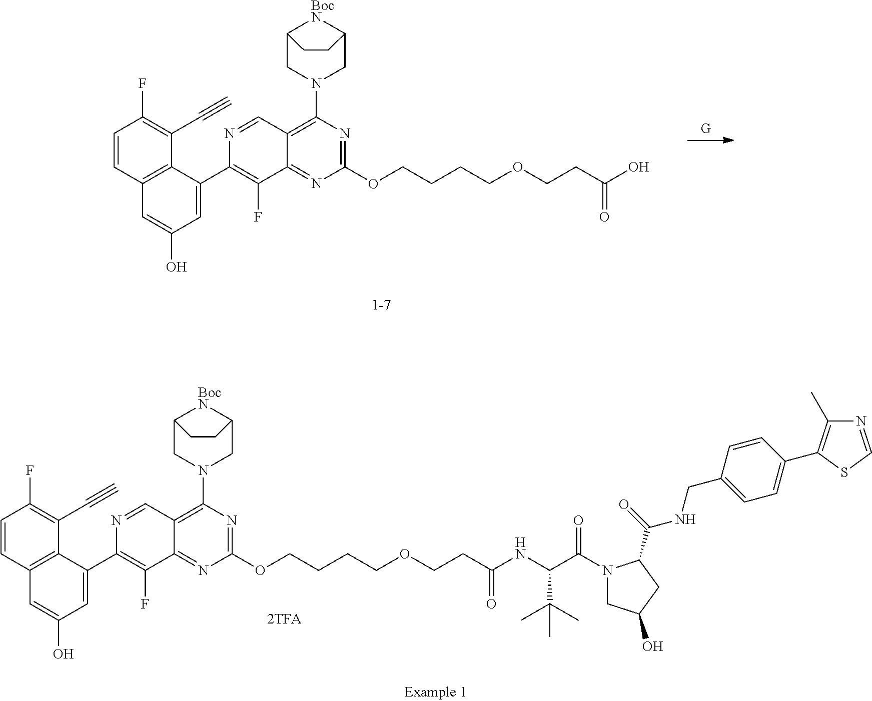

Step A: Triton B (1.11 g, 6.66 mmol, 0.3 eq) was added to a solution of isopropyl acrylate (2.84 g, 22.19 mmol, 1 eq) in 15 mL of acetonitrile, followed by addition of compound 1-1 (10 g, 110.96 mmol, 5 eq). The mixture was stirred at room temperature overnight, then concentrated. The residue was purified by column chromatography (EA/PE=1/5˜-1/1) to afford colorless oil 1-2 (3.5 g, 72% yield). H NMR (500 MHz, CDCl₃) δ 3.74-3.56 (m, 4H), 3.48 (td, J=5.6, 1.6 Hz, 2H), 2.48 (td, J=6.4, 1.6 Hz, 2H), 1.65 (qt, J=6.5, 3.8 Hz, 4H), 1.44 (s, 9H).

Step B: Compound a-1 (0.2 g, 466.98 μmol, 1 eq) was added to a solution of compound 1-2 (254.84 mg, 1.17 mmol, 2.5 eq) in 5 mL of dioxane, followed by addition of Cs₂CO₃ (456.45 mg, 1.40 mmol, 3 eq). The mixture was heated to 90° C. and stirred for 12 h, then cooled to room temperature. The mixture was treated with EtOAc and water and stirred for 10 min, then the organic layer was separated. The organic layer was washed with brine, dried over Na₂SO₄ and filtered. The filtrate was concentrated and the residue was purified by column chromatography (EA/PE=1/9˜3/2) to afford light yellow solid 1-3 (0.17 g, 60% yield). ¹H NMR (500 MHz, CDCl₃) δ 8.72 (s, 1H), 4.50-4.42 (m, 4H), 3.70-3.61 (m, 4H), 3.53-3.44 (m, 2H), 2.50-2.43 (m, 2H), 1.98-1.84 (m, 4H), 1.78-1.66 (m, 6H), 1.51 (s, 9H), 1.44 (s, 9H). m/z (ESI): 610.4 [M+H]⁺.

Step C: Compound 1-3 (0.1 g, 163.90 μmol, 1 eq) was added to a solution of ((2-fluoro-6-(methoxymethoxy)-8-(4,4,5,5-tetramethyl-1,3,2-dioxaborolan-2-yl)naphthalen-1-yl)ethynyl)triisopropylsilane (109.21 mg, 213.07 μmol, 1.3 eq) in 5 mL of dioxane/water (3/1), followed by addition of Cs₂CO₃ (160.21 mg, 491.71 μmol, 3 eq) and 1,1′-Bis(diphenylphosphino)ferrocene-palladium(II)dichloride dichloromethane complex (26.57 mg, 32.78 μmol, 0.2 eq). The mixture was stirred at 100° C. for 3 h under nitrogen atmosphere, then cooled to room temperature. The mixture was treated with EtOAc and water and stirred for 10 min, then the organic layer was separated. The organic layer was washed with brine, dried over Na₂SO₄ and filtered. The filtrate was concentrated and the residue was purified by column chromatography (EA/PE=3/7) to afford yellow solid 1-4 (60 mg, 38% yield). ¹H NMR (500 MHz, CDCl₃) δ 9.04 (s, 1H), 7.76 (dd, J=9.1, 5.6 Hz, 1H), 7.49 (d, J=2.6 Hz, 1H), 7.31-7.26 (m, 3H), 5.28 (s, 2H), 4.52-4.32 (m, 4H), 3.65 (t, J=6.5 Hz, 2H), 3.52-3.49 (m, 2H), 3.48 (s, 3H), 2.47 (t, J=6.5 Hz, 2H), 2.01-1.97 (m, 4H), 1.89 (dt, J=12.5, 6.7 Hz, 2H), 1.76 (dt, J=9.1, 6.4 Hz, 2H), 1.50 (s, 9H), 1.43 (s, 9H), 0.85 (t, J=7.9 Hz, 12H).

Step D: TBAF (27.23 mg, 104.14 μmol, 3 eq) was added to a solution of 1-4 (50 mg, 52.07 μmol, 1 eq) in 2 mL of THF. The mixture was stirred at room temperature for 10 min, then concentrated to afford crude 1-5 that was used in the next step directly. m/z (ESI): 804.4 [M+H]⁺.

Step E: TFA (17.02 mg, 149.27 μmol, 11.09 μL, 3 eq) was added to a solution of 1-5 (40 mg, 49.76 μmol, 1 eq) in 2 mL of DCM. The mixture was stirred for 30 min, then concentrated to afford crude 1-6 that used in the next step directly. m/z (ESI): 604.7 [M+H].

Step F: A solution of 1-6 (0.03 g, 49.70 μmol, 1 eq) in 2 mL of DCM was cooled to 0° C. Et₃N (5.03 mg, 49.70 μmol, 2 eq) was added to this solution slowly, followed by addition of Boc₂O (8.66 mg, 49.70 μmol, 1 eq). The mixture was stirred at room temperature for 2 h, then concentrated. The residue was purified by column chromatography (MeOH/DCM=1/9) to afford yellow oil 1-7 (30 mg, 85% yield). m/z (ESI): 704.5 [M+H]⁺.

Step G: 1-Methylimidazole (10 mg, 37.21 μmol, 5 eq) was added to a solution of 1-7 (0.032 g, 37.21 μmol, 1 eq) in 2 mL of acetonitrile, followed by addition of (2S,4R)-1-((S)-2-amino-3,3-dimethylbutanoyl)-4-hydroxy-N-(4-(4-methylthiazol-5-yl)benzyl)pyrrolidine-2-carboxamide (17.62 mg, 40.93 μmol, 1.1 eq) and N,N,N″,N″-Tetramethylchloroformamidinium-hexafluorophosphate (10.4 mg, 37.21 μmol, 1 eq). The mixture was stirred at room temperature for 2 h, then concentrated. The residue was purified by column chromatography to afford light yellow solid (20 mg, 45% yield). m/z (ESI): 1116.9 [M+H]⁺. To this solid in 3 mL of acetonitrile solution was added another solution of HCl in dioxane (0.1 mL, 4M). The mixture was stirred at room temperature for 10 min, then concentrated. The residue was purified by PRE-HPLC (0.1% TFA in water, acetonitrile) to afford light yellow solid (4.2 mg, 25% yield). ¹H NMR (500 MHz, CD₃OD) δ 9.04 (d, J=4.8 Hz, 1H), 8.88 (s, 1H), 7.88 (dd, J=9.4, 5.3 Hz, 2H), 7.45 (d, J=7.4 Hz, 3H), 7.38 (dd, J=8.7, 3.8 Hz, 3H), 7.34 (t, J=8.8 Hz, 1H), 7.26-7.18 (m, 1H), 4.76 (t, J=11.5 Hz, 4H), 4.69-4.65 (m, 1H), 4.54 (dtd, J=18.3, 13.3, 11.7, 7.0 Hz, 5H), 4.38-4.25 (m, 3H), 3.91 (dd, J=24.1, 12.1 Hz, 3H), 3.80 (d, J=10.8 Hz, 1H), 3.76-3.66 (m, 2H), 3.61-3.54 (m, 2H), 3.15 (dd, J=17.4, 8.9 Hz, 1H), 2.62-2.49 (m, 3H), 2.45 (d, J=1.7 Hz, 3H), 2.26-2.05 (m, 6H), 1.93 (t, J=7.4 Hz, 2H), 1.79 (d, J=9.5 Hz, 2H), 1.04-1.02 (m, 9H). m/z (ESI): 1016.8 [M+H]⁺.

### Example 2. Synthesis of Compound 26 Salt

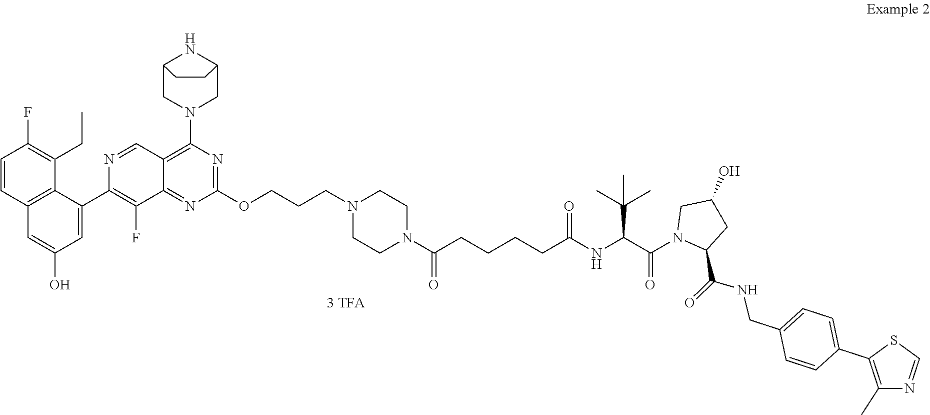 [SMILES: CCc1c(F)ccc2cc(O)cc(-c3ncc4c(N5CC6CCC(C5)N6)nc(OCCCN5CCN(C(=O)CCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 1 with intermediate f as starting material. ¹H NMR (500 MHz, CD₃OD) δ 9.09 (s, 1H), 8.87 (s, 1H), 7.66 (dd, J=9.1, 5.8 Hz, 1H), 7.45-7.36 (m, 4H), 7.29 (d, J=2.6 Hz, 1H), 7.23 (t, J=9.4 Hz, 1H), 7.02 (d, J=2.6 Hz, 1H), 4.85-4.76 (m, 4H), 4.59 (q, J=6.0 Hz, 3H), 4.55-4.44 (m, 3H), 4.33 (d, J=15.5 Hz, 1H), 4.25 (d, J=13.0 Hz, 2H), 3.99-3.85 (m, 4H), 3.78 (dd, J=10.9, 3.9 Hz, 1H), 3.58 (q, J=7.1 Hz, 2H), 3.40 (t, J=7.9 Hz, 3H), 2.49-2.40 (m, 7H), 2.28 (h, J=7.1, 5.8 Hz, 4H), 2.21-2.02 (m, 8H), 1.62 (dh, J=16.7, 6.4, 5.7 Hz, 4H), 1.15 (t, J=7.0 Hz, 2H), 1.01 (s, 9H), 0.76 (t, J=7.4 Hz, 3H). m/z (ESI): 1130.8 [M+H]⁺.

### Example 3. Synthesis of Compound 27 Salt

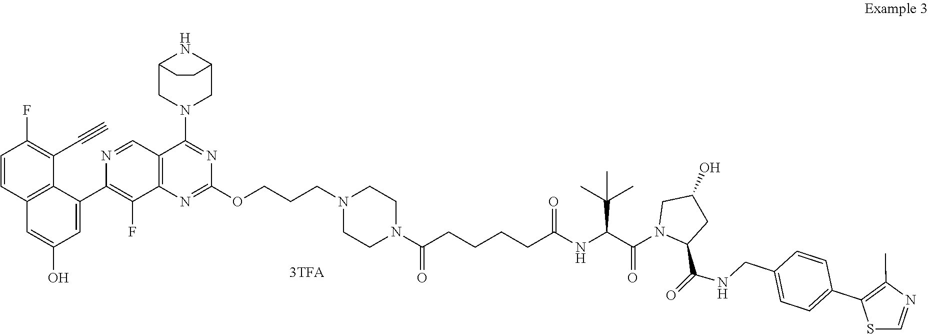 [SMILES: C#Cc1c(F)ccc2cc(O)cc(-c3ncc4c(N5CC6CCC(C5)N6)nc(OCCCN5CCN(C(=O)CCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 1 with intermediate f as starting material. ¹H NMR (500 MHz, CD₃OD) δ 9.09 (s, 1H), 9.00 (s, 1H), 7.90-7.87 (m, 1H), 7.48-7.33 (m, 7H), 7.23-7.22 (m, 1H), 4.84-4.81 (m, 2H), 4.62-4.59 (m, 3H), 4.57-4.49 (m, 3H), 4.38-4.35 (m, 1H), 4.29-4.27 (m, 2H), 4.01-3.89 (m, 2.5H), 3.82-3.79 (m, 1H), 3.74-3.60 (m, 1H), 3.53 (s, 0.5H), 3.44-3.40 (m, 3H), 3.35-3.31 (m, 4H), 2.48-2.43 (m, 5H), 2.33-2.29 (m, 4H), 2.24-2.06 (m, 6H), 1.68-1.60 (m, 4H), 1.03 (s, 9H). m/z (ESI): 1126.4 [M+H]⁺.

### Example 4. Synthesis of Compound 28 Salt

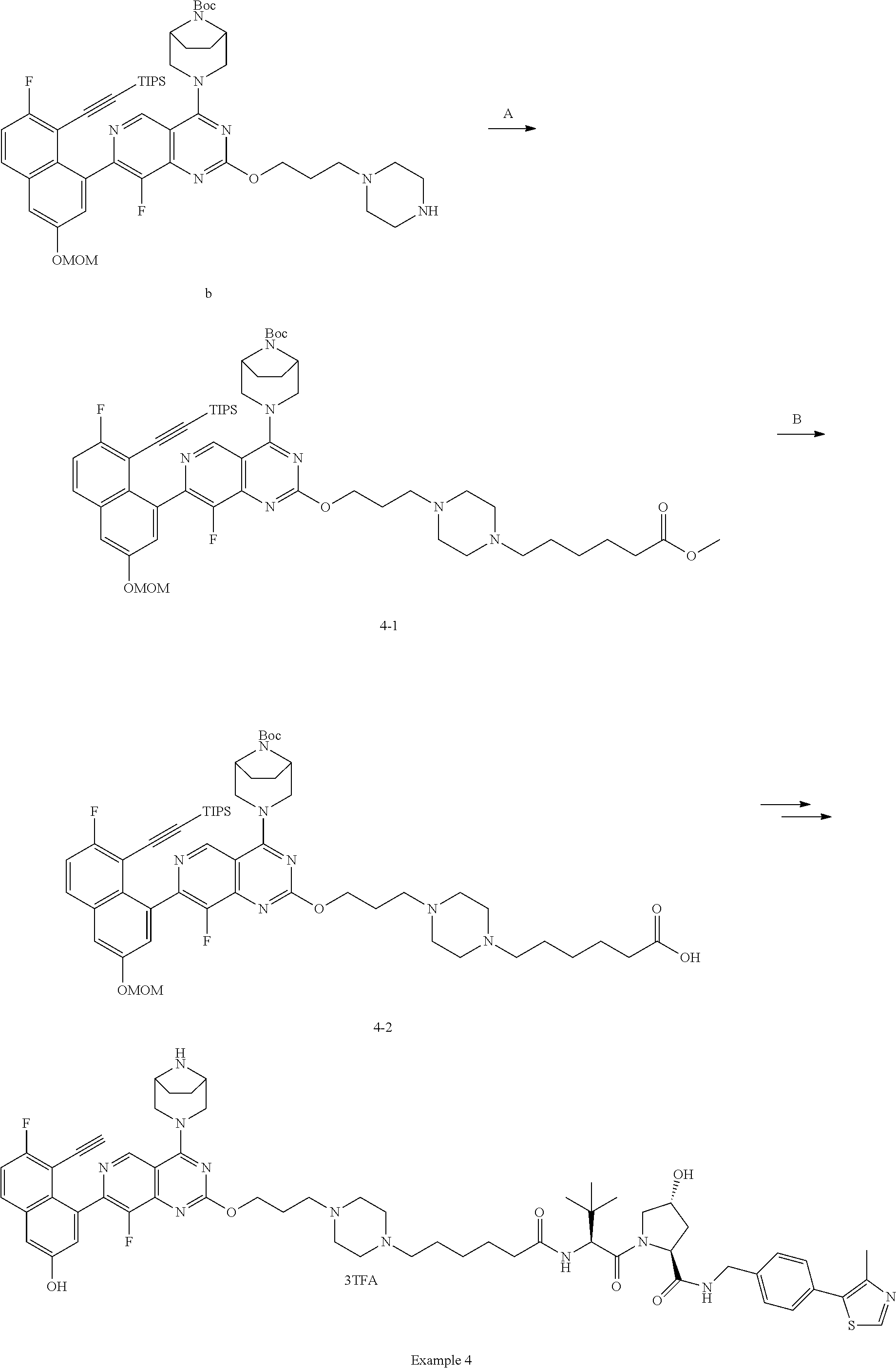

Step A: methyl 6-bromohexanoate (19.82 mg, 94.79 μmol, 1.2 eq) was added to a solution of compound b (70 mg, 78.99 μmol, 1 eq) in 10 mL of acetonitrile, followed by addition of K₂CO₃ (32.75 mg, 236.98 μmol, 3 eq) and KI (13.11, 1 μmol, 1 eq). The mixture was stirred at 60° C. for 12 h, then cooled to room temperature and filtered. The filtrate was concentrated and the residue was purified by column chromatography (MeOH/DCM=1/9) to afford yellow solid 4-1 (40 mg, 49% yield).

Step B: LiOH (4.72 mg, 197.18 μmol, 5 eq) was added to a solution of compound 4-1 (40 mg, 39.44 μmol, 1 eq) in 1 mL of THF and 1 mL of water. The mixture was heated to 50° C. and stirred for 1 h, then cooled to room temperature and concentrated. The pH of residue was adjusted to 4-5 and extracted with EA. The combined organic layer was washed with brine, dried over Na₂SO₄ and filtered. The filtrate was concentrated to afford yellow solid (35 mg, 88% yield). m/z (ESI): 1000 [M+H]⁺.

Target compound was synthesized according to the procedure of Example 1 with compound from Step B. ¹H NMR (500 MHz, CD₃OD) δ 9.09 (s, 1H), 8.97 (s, 1H), 7.90-7.87 (m, 1H), 7.48-7.41 (m, 4H), 7.39-7.33 (m, 2H), 7.23-7.23 (m, 1H), 4.87-4.82 (m, 2H), 4.64-4.60 (m, 3H), 4.57-4.50 (m, 3H), 4.38-4.35 (m, 1H), 4.29-4.27 (m, 2H), 4.01-3.90 (m, 3H), 3.82-3.79 (m, 1H), 3.57-3.36 (m, 7H), 3.26-3.22 (m, 2H), 3.16-3.13 (m, 2H), 2.48 (s, 3H), 2.32-2.21 (m, 5H), 2.15-2.07 (m, 5H), 1.77-1.64 (m, 4.5H), 1.42-1.37 (m, 2.5H), 1.04 (s, 9H). m/z (ESI): 1113.5 [M+H]⁺.

### Example 5. Synthesis of Compound 29 Salt

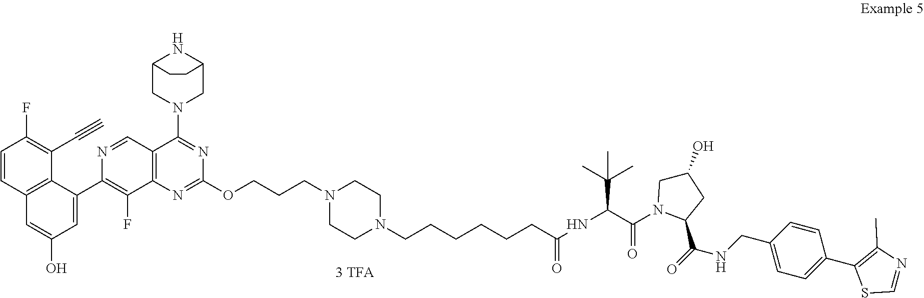 [SMILES: C#Cc1c(F)ccc2cc(O)cc(-c3ncc4c(N5CC6CCC(C5)N6)nc(OCCCN5CCN(CCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with methyl 7-bromoheptanoate as starting material. ¹H NMR (500 MHz, CD₃OD) δ 9.09 (s, 1H), 8.97 (s, 1H), 7.90-7.87 (m, 1H), 7.46-7.41 (m, 4H), 7.39-7.33 (m, 2H), 7.23-7.23 (m, 1H), 4.84 (t, J=15 Hz, 2H), 4.64-4.62 (m, 3H), 4.57-4.50 (m, 3H), 4.38-4.35 (m, 1H), 4.29-4.27 (m, 2H), 4.01-3.90 (m, 3H), 3.82-3.79 (m, 1H), 3.59-3.40 (m, 7H), 3.27-3.24 (m, 2H), 3.16-3.13 (m, 2H), 2.48 (s, 3H), 2.30-2.20 (m, 5H), 2.15-2.08 (m, 5H), 1.73-1.68 (m, 2H), 1.63-1.60 (m, 2H), 1.38 (brs, 4H), 1.03 (s, 9H). m/z (ESI): 1127.5 [M+H]⁺.

### Example 6. Synthesis of Compound 30 Salt

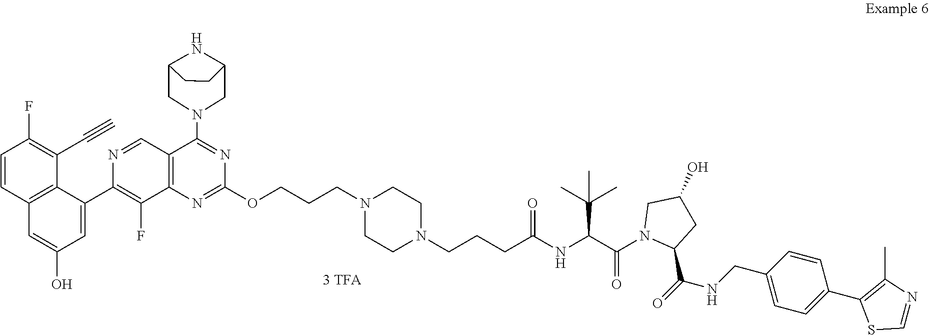 [SMILES: C#Cc1c(F)ccc2cc(O)cc(-c3ncc4c(N5CC6CCC(C5)N6)nc(OCCCN5CCN(CCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with methyl 4-bromobutanoate as starting material. ¹H NMR (500 MHz, CD₃OD) δ 9.09 (s, 1H), 8.95 (s, 1H), 7.90-7.87 (m, 1H), 7.46-7.45 (m, 2H), 7.41-7.32 (m, 4H), 7.23-7.22 (m, 1H), 4.86-4.83 (m, 3H), 4.63 (t, J=5 Hz, 2H), 4.56-4.52 (m, 3H), 4.48-4.47 (m, 1H), 4.34-4.27 (m, 3H), 4.00-3.93 (m, 3H), 3.78-3.76 (m, 1H), 3.50-3.41 (m, 6H), 3.17-3.12 (m, 3H), 2.58-2.58 (m, 2H), 2.49-2.46 (m, 3H), 2.27-2.23 (m, 3H), 2.15 (brs, 4H), 2.10-2.04 (m, 1H), 1.99-1.98 (m, 2H), 1.06 (s, 9H). m/z (ESI): 1085.5 [M+H]⁺.

### Example 7. Synthesis of Compound 31 Salt

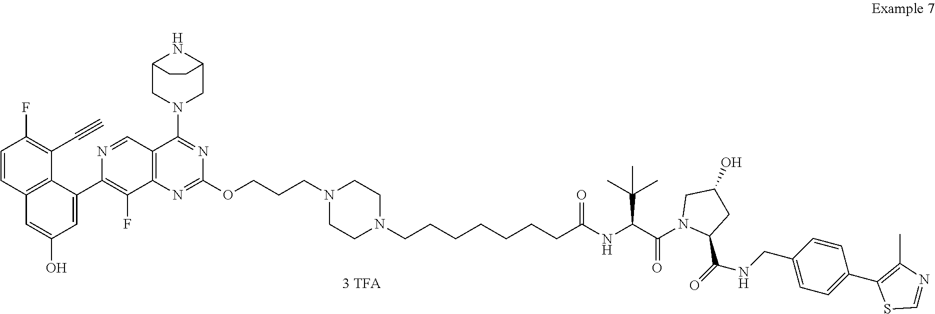 [SMILES: C#Cc1c(F)ccc2cc(O)cc(-c3ncc4c(N5CC6CCC(C5)N6)nc(OCCCN5CCN(CCCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with methyl 8-bromooctanoate as starting material. ¹H NMR (500 MHz, CD₃OD) δ 9.06 (s, 1H), 8.92 (s, 1H), 7.90-7.87 (m, 1H), 7.48-7.41 (m, 4H), 7.38-7.33 (m, 2H), 7.22-7.21 (m, 1H), 4.83 (m, 2H), 4.64-4.50 (m, 6H), 4.38-4.35 (m, 1H), 4.28-4.26 (m, 2H), 3.98-3.90 (m, 3H), 3.82-3.79 (m, 1H), 3.46-3.35 (m, 7H), 3.08-3.05 (m, 2H), 3.02-2.99 (m, 2H), 2.47 (s, 3H), 2.31-2.04 (m, 10H), 1.71-1.58 (m, 4H), 1.36 (brs, 6H), 1.03 (s, 9H). m/z (ESI): 1141.5 [M+H]⁺.

### Example 8. Synthesis of Compound 32 Salt

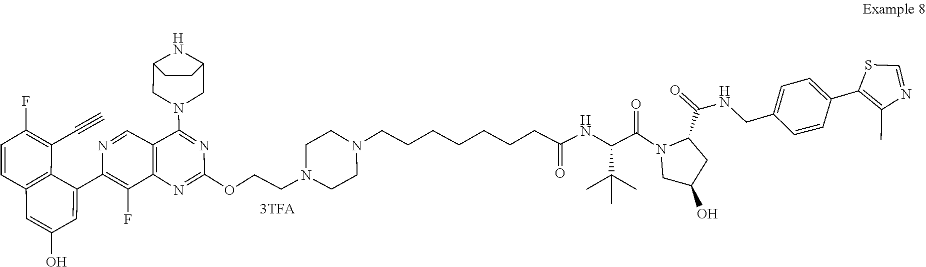 [SMILES: C#Cc1c(F)ccc2cc(O)cc(-c3ncc4c(N5CC6CCC(C5)N6)nc(OCCN5CCN(CCCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate e as starting material. ¹H NMR (500 MHz, CD₃OD) δ 9.09 (s, 1H), 8.96 (s, 1H), 7.91-7.83 (m, 2H), 7.48-7.33 (m, 6H), 7.23-7.22 (m, 1H), 4.89-4.78 (m, 3H), 4.67-4.63 (m, 2H), 4.57-4.50 (m, 3H), 4.38-4.35 (m, 1H), 4.29-4.26 (m, 2H), 4.01-3.98 (m, 1H), 3.91-3.88 (m, 2H), 3.82-3.79 (m, 1H), 3.46 (s, 1H), 3.45-3.37 (m, 2H), 3.25-3.09 (m, 5H), 2.48 (s, 3H), 2.32-2.05 (m, 9H), 1.68-1.59 (m, 4H), 1.36 (brs, 6H), 1.03 (s, 9H). m/z (ESI): 1126.4 [M+H]⁺.

### Example 9. Synthesis of Compound 33 Salt

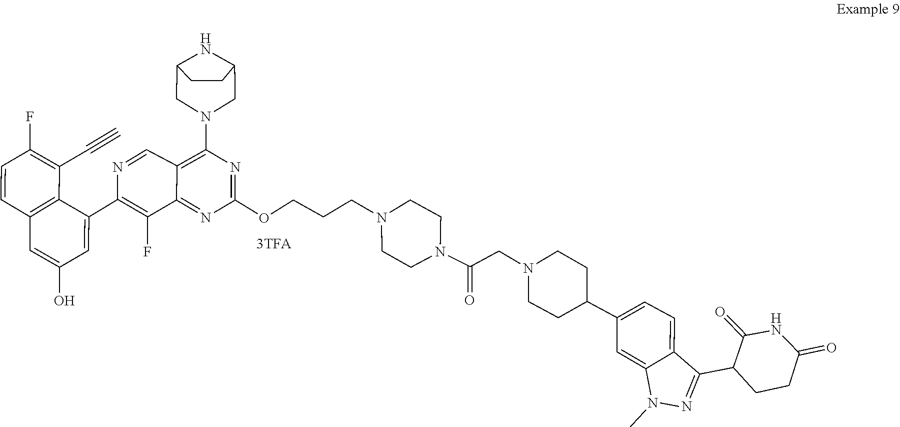 [SMILES: C#Cc1c(F)ccc2cc(O)cc(-c3ncc4c(N5CC6CCC(C5)N6)nc(OCCCN5CCN(C(=O)CN6CCC(c7ccc8c(C9CCC(=O)NC9=O)nn(C)c8c7)CC6)CC5)nc4c3F)c12]

Target compound was synthesized according to the procedure of Example 4. ¹H NMR (500 MHz, DMSO-d6) δ 11.65 (s, 1H), 10.92 (s, 1H), 9.94-9.92 (m, 1H), 9.80 (s, 1H), 9.63 (s, 1H), 9.12 (s, 1H), 8.00-7.98 (m, 1H), 7.96-7.67 (m, 1H), 7.49-7.41 (m, 3H), 7.23 (s, 1H), 7.06-7.05 (m, 1H), 4.52-4.50 (m, 1H), 4.51-4.50 (m, 5H), 4.36-4.34 (m, 2H), 4.20-4.19 (m, 2H), 4.00-3.99 (m, 6H), 3.30 (s, 2H), 3.32-3.18 (m, 2H), 3.07-2.99 (m, 2.5H), 2.90-2.87 (m, 0.5H), 2.64-2.58 (m, 2H), 2.36-2.35 (m, 1H), 2.30-2.27 (m, 2H), 2.18-2.15 (m, 3H), 2.07-1.94 (m, 7H), 1.23-1.16 (m, 5H). m/z (ESI): 952.7 [M+H]⁺.

### Example 10. Synthesis of Compound 34 Salt

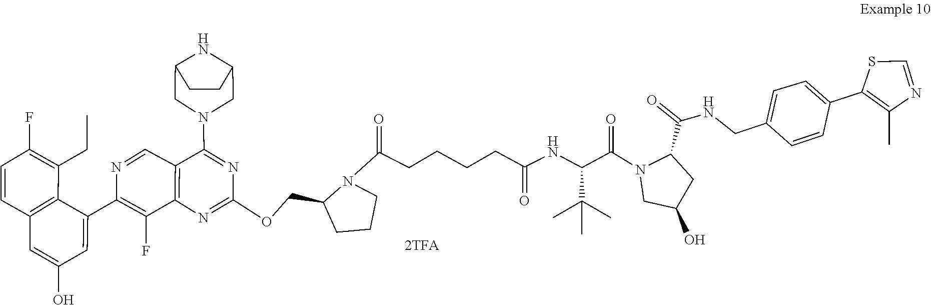 [SMILES: CCc1c(F)ccc2cc(O)cc(-c3ncc4c(N5CC6CCC(C5)N6)nc(OC[C@@H]5CCCN5C(=O)CCCCC(=O)N[C@H](C(=O)N5C[C@H](O)C[C@H]5C(=O)NCc5ccc(-c6scnc6C)cc5)C(C)(C)C)nc4c3F)c12]

Target compound was synthesized according to the procedure of Example 2 with intermediate d as starting material. H NMR (500 MHz, CD₃OD) δ 8.93 (s, 1H), 8.90 (s, 1H), 7.72-7.69 (m, 1H), 7.47-7.41 (m, 4H), 7.35 (s, 1H), 7.29 (t, J=10 Hz, 1H), 7.1-7.10 (m, 1H), 4.87-4.69 (m, 4H), 4.64-4.47 (m, 6H), 4.38-4.23 (m, 4H), 4.09-3.73 (m, 8H), 2.62-2.55 (m, 1H), 2.47 (s, 3H), 2.43-2.38 (m, 2H), 2.20-2.09 (m, 8H), 1.68-1.59 (m, 4H), 1.32-1.30 (m, 1H), 1.04 (s, 9H), 0.86 (t, J=5 Hz, 3H). m/z (ESI): 1087.8 [M+H]⁺.

### Example 11. Synthesis of Compound 35 Salt

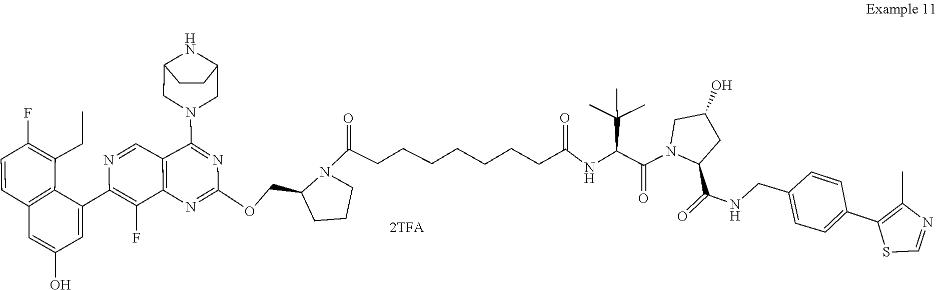 [SMILES: CCc1c(F)ccc2cc(O)cc(-c3ncc4c(N5CC6CCC(C5)N6)nc(OC[C@@H]5CCCN5C(=O)CCCCCCCC(=O)N[C@H](C(=O)N5C[C@H](O)C[C@H]5C(=O)NCc5ccc(-c6scnc6C)cc5)C(C)(C)C)nc4c3F)c12]

Target compound was synthesized according to the procedure of Example 2 with intermediate d as starting material. ¹H NMR (500 MHz, CD₃OD) δ 8.92-8.91 (m, 2H), 7.171 (t, J=5 Hz, 1H), 7.47-7.40 (m, 4H), 7.35 (brs, 1H), 7.28 (t, J=10 Hz, 1H), 7.16-7.11 (m, 1H), 4.68-4.62 (m, 5H), 4.36-4.26 (m, 3H), 3.95-7.79 (m, 5H), 2.60 (brs, 1H), 2.47 (s, 3H), 2.37 (t, J=5 Hz, 1H), 2.24-2.16 (m, 10H), 1.66-1.52 (m, 5H), 1.33-1.30 (m, 14H), 1.02 (s, 9H), 0.86 (brs, 3H). m/z (ESI): 1129.9 [M+H]⁺.

### Example 12. Synthesis of Compound 36 Salt

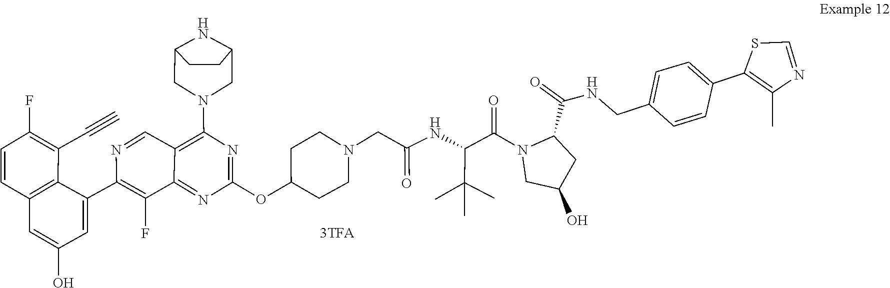 [SMILES: C#Cc1c(F)ccc2cc(O)cc(-c3ncc4c(N5CC6CCC(C5)N6)nc(OC5CCN(CC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.09 (s, 1H), 8.94 (s, 1H), 7.89-7.86 (m, 1H), 7.37-7.35 (m, 2H), 7.34-7.32 (m, 4H), 7.22 (s, 1H), 5.49 (s, 2H), 4.81-4.79 (m, 2H), 4.65 (s, 1H), 4.56-4.52 (m, 3H), 4.37-4.29 (m, 3H), 4.12-3.92 (m, 4H), 3.82-3.80 (m, 1H), 3.39 (s, 1H), 2.47 (s, 3H), 2.21-2.02 (m, 8H), 1.35-1.29 (m, 9H), 1.07 (s, 9H). m/z (ESI): 1129.9 [M+H]⁺.

### Example 13. Synthesis of Compound 37 Salt

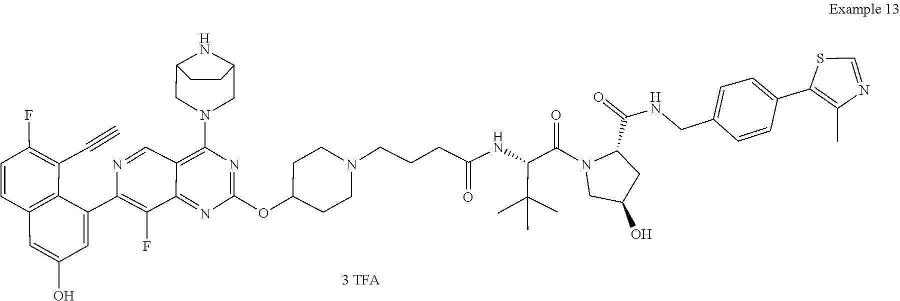 [SMILES: C#Cc1c(F)ccc2cc(O)cc(-c3ncc4c(N5CC6CCC(C5)N6)nc(OC5CCN(CCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.08 (s, 1H), 8.90 (s, 1H), 7.90-7.87 (m, 1H), 7.45-7.36 (m, 2H), 7.34-7.32 (m, 5H), 7.21 (s, 1H), 4.88-4.78 (m, 2H), 4.57-4.51 (m, 4H), 4.35-4.28 (m, 3H), 3.96-3.94 (m, 3H), 3.81-3.79 (m, 1H), 3.59-3.51 (m, 2H), 3.37 (s, 1H), 3.24-3.17 (m, 3H), 2.58-2.53 (m, 2H), 2.46 (s, 3H), 2.42-2.36 (m, 2H), 2.28-2.21 (m, 2H), 2.16-2.04 (m, 8H), 1.07 (s, 9H). m/z (ESI): 1041.6 [M+H]⁺.

### Example 14. Synthesis of Compound 38 Salt

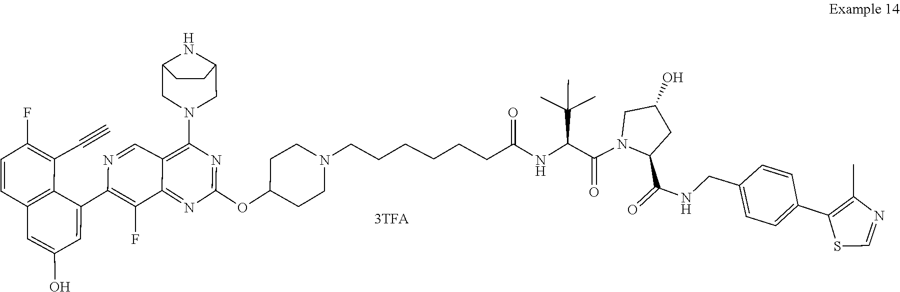 [SMILES: C#Cc1c(F)ccc2cc(O)cc(-c3ncc4c(N5CC6CCC(C5)N6)nc(OC5CCN(CCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.06 (s, 1H), 8.90 (s, 1H), 7.86 (dd, J=9.2, 5.7 Hz, 1H), 7.45-7.28 (m, 7H), 7.19 (d, J=2.5 Hz, 1H), 5.47 (s, 1H), 4.77 (t, J=13.6 Hz, 2H), 4.62 (t, J=4.2 Hz, 1H), 4.57-4.44 (m, 3H), 4.38-4.23 (m, 3H), 3.99-3.92 (m, 2H), 3.89 (d, J=11.7 Hz, 1H), 3.78 (dd, J=11.0, 3.9 Hz, 1H), 3.60 (dd, J=79.0, 12.6 Hz, 2H), 3.37 (s, 2H), 3.17 (dt, J=38.8, 14.4 Hz, 3H), 2.50 (d, J=14.2 Hz, 1H), 2.45 (s, 3H), 2.39-2.25 (m, 4H), 2.23-2.03 (m, 9H), 1.75 (s, 2H), 1.62 (s, 2H), 1.39 (s, 4H), 1.01 (s, 9H). m/z (ESI): 1127.5 [M+H]⁺.

### Example 15. Synthesis of Compound 39 Salt

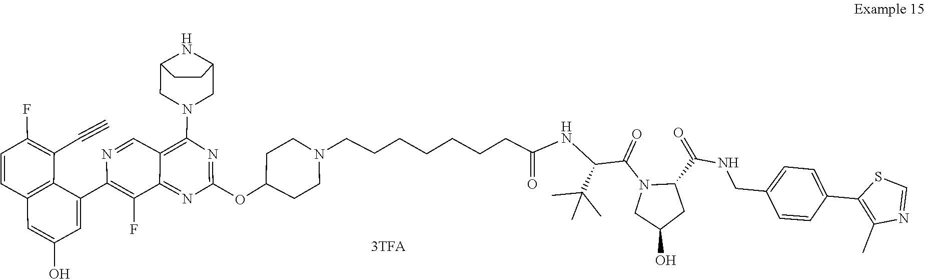 [SMILES: C#Cc1c(F)ccc2cc(O)cc(-c3ncc4c(N5CC6CCC(C5)N6)nc(OC5CCN(CCCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.07 (s, 1H), 8.93 (s, 1H), 7.86 (dd, J=9.2, 5.7 Hz, 1H), 7.47-7.29 (m, 7H), 7.19 (d, J=2.6 Hz, 1H), 5.47 (s, 2H), 4.79 (d, J=16.1 Hz, 3H), 4.62 (s, 1H), 4.56-4.46 (m, 3H), 4.37-4.23 (m, 3H), 3.99-3.85 (m, 3H), 3.81-3.75 (m, 1H), 3.60 (dd, J=78.4, 13.1 Hz, 2H), 3.34 (d, J=21.4 Hz, 2H), 3.19-3.09 (m, 3H), 2.50 (d, J=14.3 Hz, 1H), 2.45 (s, 3H), 2.39-1.95 (m, 12H), 1.75 (s, 2H), 1.60 (d, J=10.2 Hz, 2H), 1.38 (s, 6H), 1.01 (s, 9H). m/z (ESI): 1127.5 [M+H]⁺.

### Example 16. Synthesis of Compound 40 Salt

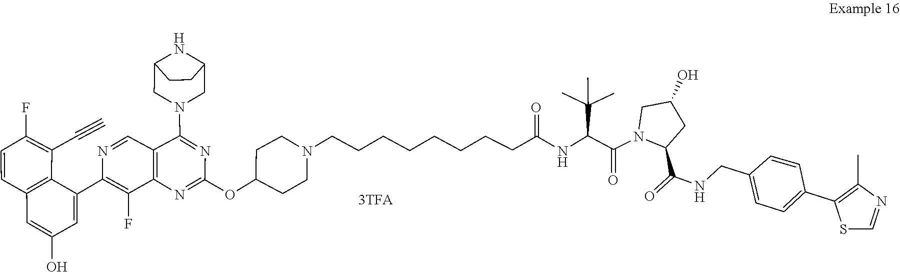 [SMILES: C#Cc1c(F)ccc2cc(O)cc(-c3ncc4c(N5CC6CCC(C5)N6)nc(OC5CCN(CCCCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.08-9.07 (m, 1H), 8.91 (s, 1H), 7.90-7.84 (m, 1H), 7.47-7.46 (m, 2H), 7.42-7.40 (m, 2H), 7.38-7.32 (m, 2H), 7.21 (s, 1H), 5.60-5.43 (m, 1H), 4.86-4.80 (m, 2H), 4.65-4.63 (m, 1H), 4.57-4.50 (m, 3H), 4.37-4.34 (m, 1H), 4.28 (brs, 2H), 3.99-3.89 (m, 3H), 3.82-3.80 (m, 1H), 3.39 (s, 1H), 3.19-3.11 (m, 7H), 2.48 (s, 3H), 2.29-2.08 (m, 10H), 1.77 (brs, 2H), 1.71-1.65 (m, 5H), 1.61 (brs, 2H), 1.46-1.39 (m, 10H), 1.03 (s, 9H). m/z (ESI): 1111.3 [M+H]⁺.

### Example 17. Synthesis of Compound 41 Salt

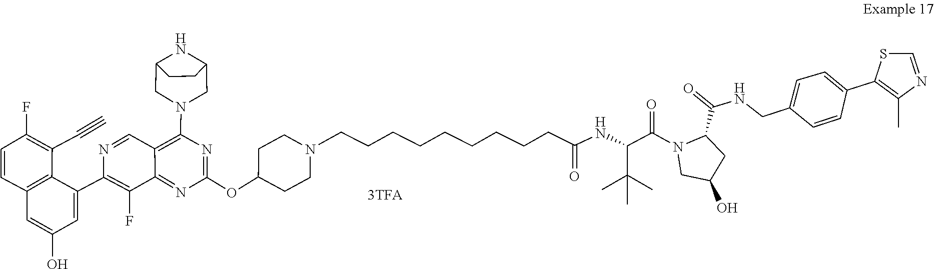 [SMILES: C#Cc1c(F)ccc2cc(O)cc(-c3ncc4c(N5CC6CCC(C5)N6)nc(OC5CCN(CCCCCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.08-9.07 (m, 1H), 8.91 (s, 1H), 7.90-7.87 (m, 1H), 7.82-7.80 (d, J=10 Hz, 1H), 7.47-7.46 (m, 2H), 7.44-7.40 (m, 2H), 7.37-7.32 (m, 2H), 7.21 (s, 1H), 4.83-4.79 (m, 2H), 4.65-4.63 (m, 1H), 4.57-4.50 (m, 3H), 4.37-4.34 (m, 1H), 4.28 (brs, 2H), 3.98-3.94 (m, 3H), 3.82-3.80 (m, 1H), 3.71-3.69 (m, 1H), 3.56-3.53 (m, 1H), 3.38 (s, 1H), 3.35 (s, 1H), 3.25-3.16 (m, 3H), 2.54-2.52 (m, 1H), 2.48 (s, 3H), 2.39-2.36 (m, 1H), 2.29-2.00 (m, 10H), 1.77 (brs, 2H), 1.61 (brs, 2H), 1.39-1.35 (m, 10H), 1.03 (s, 9H). m/z (ESI): 1125.7 [M+H]⁺.

### Example 18. Synthesis of Compound 42 Salt

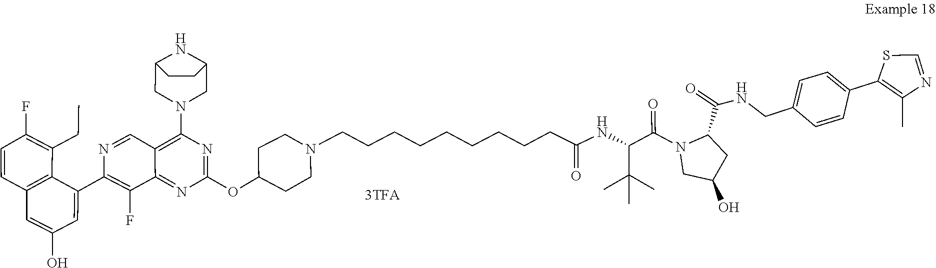 [SMILES: CCc1c(F)ccc2cc(O)cc(-c3ncc4c(N5CC6CCC(C5)N6)nc(OC5CCN(CCCCCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with Example 17 as starting material. m/z (ESI): 1129.6 [M+H]⁺.

### Example 19. Synthesis of Compound 43 Salt

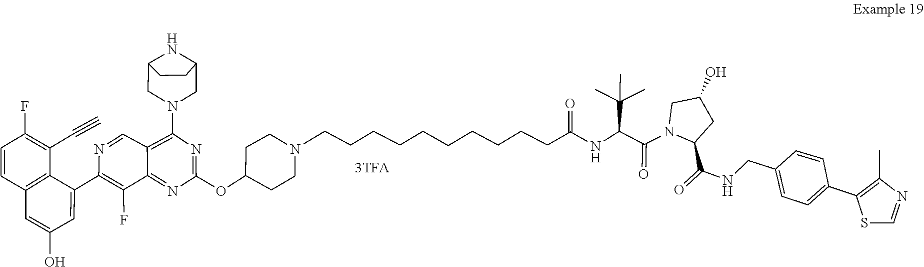 [SMILES: C#Cc1c(F)ccc2cc(O)cc(-c3ncc4c(N5CC6CCC(C5)N6)nc(OC5CCN(CCCCCCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.10 (s, 1H), 8.97 (s, 1H), 7.90-7.87 (m, 1H), 7.48-7.46 (m, 2H), 7.45-7.43 (m, 2H), 7.38-7.33 (m, 2H), 7.22 (s, 1H), 5.60-5.34 (m, 1H), 4.86-4.80 (m, 2H), 4.64 (s, 1H), 4.59-4.50 (m, 3H), 4.37-4.33 (m, 1H), 4.29 (brs, 2H), 4.01-3.89 (m, 3H), 3.82-3.79 (m, 1H), 3.72-3.62 (m, 1H), 3.56-3.53 (m, 1H), 3.41 (s, 1H), 3.37-3.34 (m, 1H), 3.20-3.11 (m, 3H), 2.48 (s, 3H), 2.39-2.04 (m, 12H), 1.77 (brs, 2H), 1.70-1.66 (m, 1H), 1.60 (brs, 2H), 1.43-1.33 (m, 14H), 1.03 (s, 9H). m/z (ESI): 1139.3 [M+H]⁺.

### Example 20. Synthesis of Compound 44 Salt

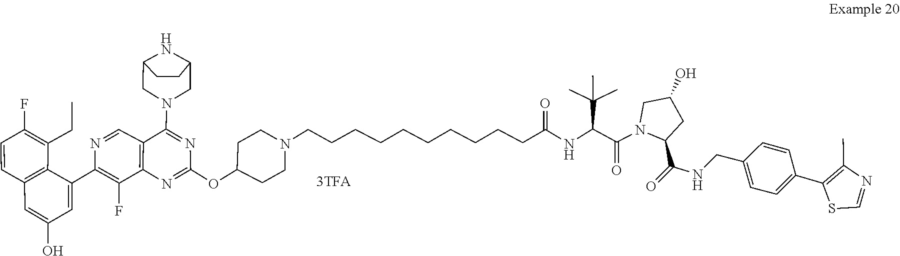 [SMILES: CCc1c(F)ccc2cc(O)cc(-c3ncc4c(N5CC6CCC(C5)N6)nc(OC5CCN(CCCCCCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with Example 19 as starting material. ¹H NMR (500 MHz, CD₃OD) δ 9.12-9.11 (m, 1H), 8.91 (s, 1H), 7.71-7.68 (dd, J1=5 Hz, J2=10 Hz, 1H), 7.47-7.41 (m, 4H), 7.32-7.32 (m, 1H), 7.27 (t, J=10 Hz, 1H), 7.04-7.03 (m, 1H), 5.60 (brs, 0.6H), 5.45-5.43 (m, 0.4H), 4.87-4.76 (m, 3H), 4.65-4.64 (m, 1H), 4.57-4.50 (m, 3H), 4.37-4.34 (m, 1H), 4.29-4.26 (m, 2H), 4.00-3.89 (m, 3H), 3.82-3.79 (m, 1H), 3.72-3.69 (m, 1H), 3.56-3.54 (m, 1H), 3.38-3.35 (m, 2H), 3.26-3.121 (m, 3H), 2.48 (s, 3H), 2.41-2.36 (m, 1H), 2.33-2.08 (m, 10H), 1.77 (brs, 2H), 1.60 (brs, 2H), 1.40-1.30 (m, 14H), 1.03 (s, 9H), 0.81-0.78 (m, 3H). m/z (ESI): 1143.5 [M+H]⁺.

### Example 21. Synthesis of Compound 45 Salt

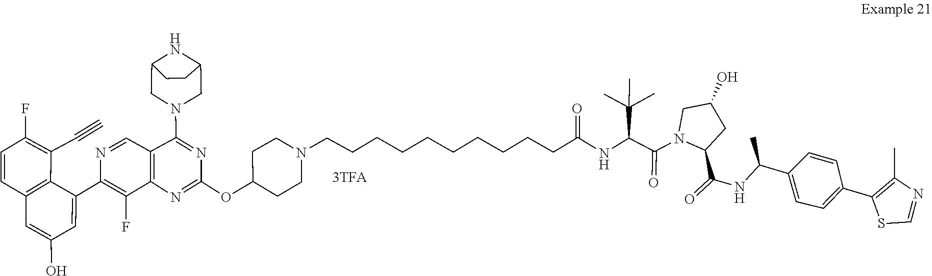 [SMILES: C#Cc1c(F)ccc2cc(O)cc(-c3ncc4c(N5CC6CCC(C5)N6)nc(OC5CCN(CCCCCCCCCCC(=O)N[C@H](C(=O)N6C[C@H](O)C[C@H]6C(=O)N[C@@H](C)c6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.12-9.07 (m, 1H), 8.95 (s, 1H), 8.62-8.60 (m, 1H), 7.90-7.85 (m, 2H), 7.46-7.41 (m, 4H), 7.38-7.33 (m, 2H), 7.21-7.21 (m, 1H), 5.60 (s, 0.7H), 5.45-5.41 (m, 0.3H), 5.01-4.99 (m, 2H), 4.86-4.79 (m, 2H), 4.64-4.62 (m, 1H), 4.57-4.54 (m, 1H), 4.43 (s, 1H), 4.28 (s, 2H), 3.99-3.94 (m, 2H), 3.88-3.86 (m, 1H), 3.76-3.69 (m, 2H), 3.56-3.54 (m, 1.5H), 3.40-3.35 (m, 2H), 3.25-3.16 (m, 3.5H), 2.54-2.52 (m, 1H), 2.48 (s, 3H), 2.39-2.21 (m, 10H), 2.04-1.91 (m, 2H), 1.77 (brs, 2H), 1.61-1.56 (m, 2H), 1.50 (d, J=5 Hz, 3H), 1.39-1.33 (m, 14H), 1.04 (s, 9H). m/z (ESI): 1153.7 [M+H]⁺.

### Example 22. Synthesis of Compound 46 Salt

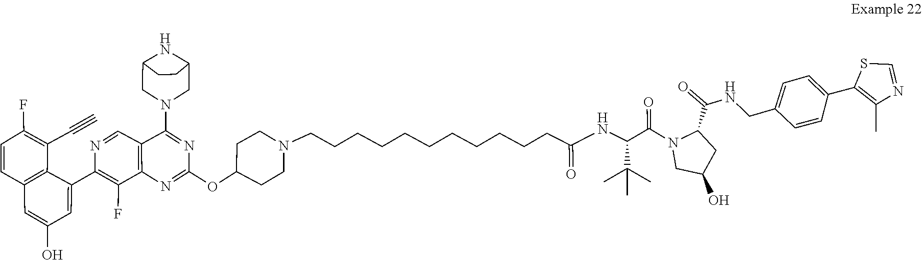 [SMILES: C#Cc1c(F)ccc2cc(O)cc(-c3ncc4c(N5CC6CCC(C5)N6)nc(OC5CCN(CCCCCCCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.08-9.07 (m, 1H), 8.92 (s, 1H), 7.90-7.85 (m, 2H), 7.48-7.45 (m, 2H), 7.43-7.40 (m, 2H), 7.38-7.33 (m, 2H), 7.21-7.20 (m, 1H), 5.60 (s, 0.7H), 5.50-5.40 (m, 0.3H), 4.86-4.77 (m, 2H), 4.65-4.63 (m, 1H), 4.57-4.50 (m, 3H), 4.38-4.34 (m, 1H), 4.28 (brs, 2H), 3.98-3.89 (m, 3H), 3.82-3.79 (m, 1H), 3.72-3.69 (m, 1H), 3.56-3.53 (m, 1.6H), 3.45-3.44 (m, 0.4H), 3.39 (s, 2H), 3.25-3.16 (m, 3H), 2.55-2.52 (m, 1H), 2.48 (s, 3H), 2.40-2.26 (m, 3H), 2.25-2.20 (m, 3H), 2.11-1.99 (m, 2H), 1.77 (brs, 2H), 1.60 (brs, 2H), 1.39-1.32 (m, 18H), 1.03 (s, 9H). m/z (ESI): 1153.7 [M+H]⁺.

### Example 23. Synthesis of Compound 47 Salt

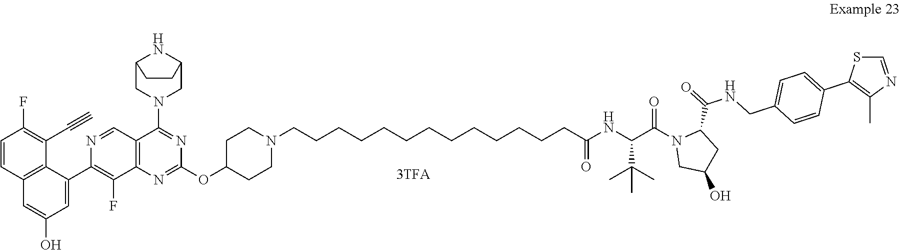 [SMILES: C#Cc1c(F)ccc2cc(O)cc(-c3ncc4c(N5CC6CCC(C5)N6)nc(OC5CCN(CCCCCCCCCCCCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.08-9.07 (m, 1H), 8.92 (s, 1H), 7.90-7.78 (m, 2H), 7.48-7.41 (m, 4H), 7.38-7.33 (m, 2H), 7.21-7.20 (m, 1H), 5.60 (s, 0.6H), 5.45-5.41 (m, 0.4H), 4.87-4.77 (m, 2H), 4.64-4.63 (m, 1H), 4.57-4.50 (m, 3H), 4.37-4.34 (m, 1H), 4.28 (brs, 2H), 3.98-3.89 (m, 3H), 3.82-3.79 (m, 1H), 3.72-3.69 (m, 0.8H), 3.63-3.62 (m, 0.2H), 3.56-3.53 (m, 1H), 3.39-3.35 (m, 2H), 3.25-3.13 (m, 3H), 2.55-2.52 (m, 0.7H), 2.48 (s, 3H), 2.40-2.36 (m, 1.3H), 2.33-2.20 (m, 4H), 2.10-1.99 (m, 3H), 1.77 (brs, 2H), 1.60 (brs, 2H), 1.39-1.30 (m, 20H), 1.03 (s, 9H). m/z (ESI): 1181.5 [M+H]⁺.

### Example 24. Synthesis of Compound 48 Salt

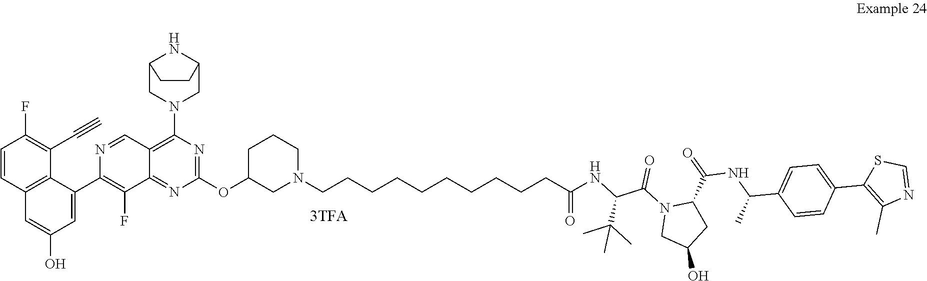 [SMILES: C#Cc1c(F)ccc2cc(O)cc(-c3ncc4c(N5CC6CCC(C5)N6)nc(OC5CCCN(CCCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12]

Target compound was synthesized according to the procedure of Example 4 with intermediate c as starting material. ¹H NMR (500 MHz, CD₃OD) δ 9.10-8.95 (m, 1H), 8.86 (s, 1H), 7.79 (dd, J=9.4, 5.9 Hz, 1H), 7.33 (q, J=8.2 Hz, 4H), 7.29-7.22 (m, 2H), 7.12 (d, J=3.2 Hz, 1H), 4.79-4.66 (m, 2H), 4.55-4.42 (m, 2H), 4.33 (s, 1H), 4.18 (s, 2H), 3.96-3.74 (m, 4H), 3.64 (d, J=10.9 Hz, 1H), 3.55-3.41 (m, 1H), 3.38-3.24 (m, 2H), 3.16-2.87 (m, 3H), 2.39 (s, 3H), 2.23-1.97 (m, 10H), 1.90-1.79 (m, 2H), 1.64 (s, 2H), 1.47 (d, J=7.7 Hz, 2H), 1.40 (d, J=7.4 Hz, 3H), 1.33-1.13 (m, 14H), 0.93 (s, 9H). m/z (ESI): 1153.8 [M+H]⁺.

### Example 25. Synthesis of Compound 49 Salt

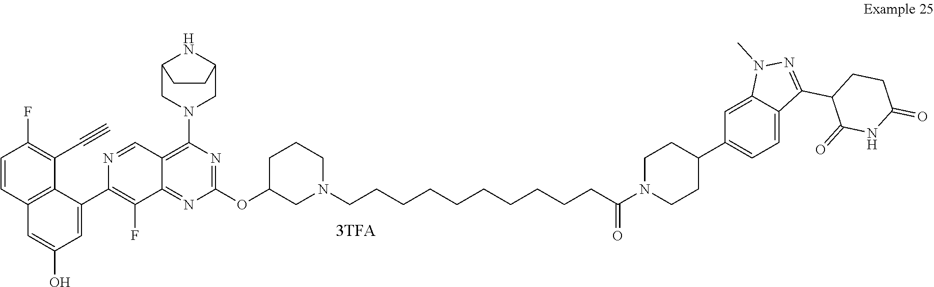 [SMILES: C#Cc1c(F)ccc2cc(O)cc(-c3ncc4c(N5CC6CCC(C5)N6)nc(OC5CCCN(CCCCCCCCCCC(=O)N6CCC(c7ccc8c(C9CCC(=O)NC9=O)nn(C)c8c7)CC6)C5)nc4c3F)c12]

Target compound was synthesized according to the procedure of Example 4 with intermediate c as starting material. ¹H NMR (500 MHz, CD₃OD) δ 9.04 (s, 1H), 7.91-7.76 (m, 1H), 7.59 (d, J=8.5 Hz, 1H), 7.42-7.23 (m, 3H), 7.17 (s, 1H), 7.01 (d, J=8.5 Hz, 1H), 5.66 (s, 1H), 4.82-4.70 (m, 2H), 4.66 (d, J=13.2 Hz, 1H), 4.30 (t, J=7.4 Hz, 1H), 4.23 (s, 2H), 4.05 (d, J=7.7 Hz, 1H), 3.95 (s, 3H), 3.92-3.76 (m, 2H), 3.55-3.48 (m, 1H), 3.34 (d, J=13.8 Hz, 1H), 3.22-2.89 (m, 6H), 2.70 (d, J=11.6 Hz, 3H), 2.46-2.32 (m, 3H), 2.25 (dd, J=13.4, 6.0 Hz, 2H), 2.16-2.02 (m, 4H), 1.89 (d, J=15.6 Hz, 4H), 1.75-1.51 (m, 7H), 1.38-1.24 (m, 12H). m/z (ESI): 1035.3 [M+H]⁺.

### Example 26. Synthesis of Compound 50 Salt

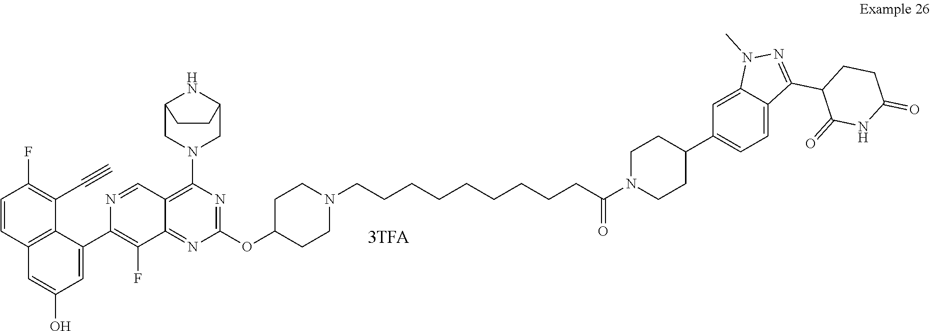 [SMILES: C#Cc1c(F)ccc2cc(O)cc(-c3ncc4c(N5CC6CCC(C5)N6)nc(OC5CCN(CCCCCCCCCC(=O)N6CCC(c7ccc8c(C9CCC(=O)NC9=O)nn(C)c8c7)CC6)CC5)nc4c3F)c12]

Target compound was synthesized according to the procedure of Example 4 with intermediate a as starting material. ¹H NMR (500 MHz, CD₃OD) δ 9.08-9.07 (m, 1H), 7.89-7.86 (m, 1H), 7.66-7.64 (m, 1H), 7.37-7.32 (m, 3H), 7.21-7.20 (m, 1H), 7.09-7.07 (m, 1H), 5.62-5.44 (m, 1H), 4.82-4.71 (m, 3H), 4.37-4.34 (m, 1H), 4.28 (brs, 2H), 4.14-4.11 (m, 1H), 4.00 (s, 3H), 3.98-3.90 (m, 2H), 3.69-3.67 (m, 1H), 3.54-3.52 (m, 1H), 3.38-3.36 (m, 1H), 3.28-3.20 (m, 4H), 3.03-2.96 (m, 1H), 2.79-2.72 (m, 3H), 2.54-2.29 (m, 6H), 2.22-2.11 (m, 5H), 2.04-1.94 (m, 3H), 1.78-1.62 (m, 6H), 1.40 (brs, 10H). m/z (ESI): 1021.3 [M+H]⁺.

### Example 27. Synthesis of Compound 51 Salt

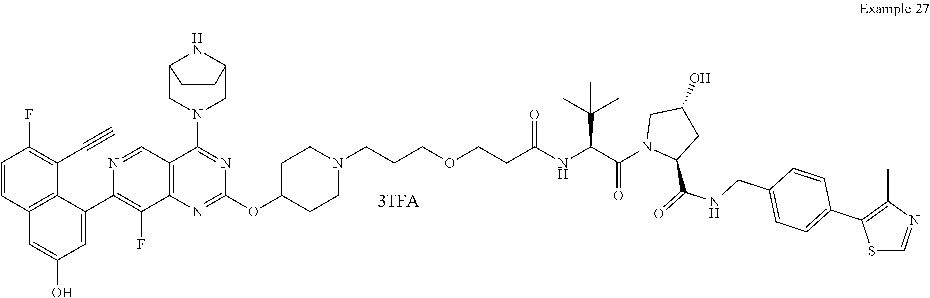 [SMILES: C#Cc1c(F)ccc2cc(O)cc(-c3ncc4c(N5CC6CCC(C5)N6)nc(OC5CCN(CCCOCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.09-9.08 (m, 1H), 8.94 (s, 1H), 8.00 (t. J=5 Hz, 1H), 7.89-7.86 (m, 1H), 7.42-7.40 (m, 2H), 7.37-7.30 (m, 4H), 7.22 (s, 1H), 5.58-5.42 (m, 1H), 4.86-4.76 (m, 2H), 4.68-4.64 (m, 1H), 4.59-4.56 (m, 1H), 4.49-4.46 (m, 2H), 4.38-4.33 (m, 1H), 4.28 (brs, 2H), 4.01-3.93 (m, 2H), 3.90-3.86 (m, 1H), 3.82-3.80 (m, 2H), 3.72-3.71 (m, 2H), 3.64-3.62 (m, 3H), 3.40-3.35 (m, 2H), 3.24-3.17 (m, 1H), 2.57-2.55 (m, 2H), 2.50-2.48 (m, 1H), 2.46-2.45 (m, 3H), 2.34-2.23 (m, 3H), 2.18-2.06 (m, 7H), 2.04 (s, 2H), 1.06 (s, 9H). m/z (ESI): 1085.5 [M+H]⁺.

### Example 28. Synthesis of Compound 52 Salt

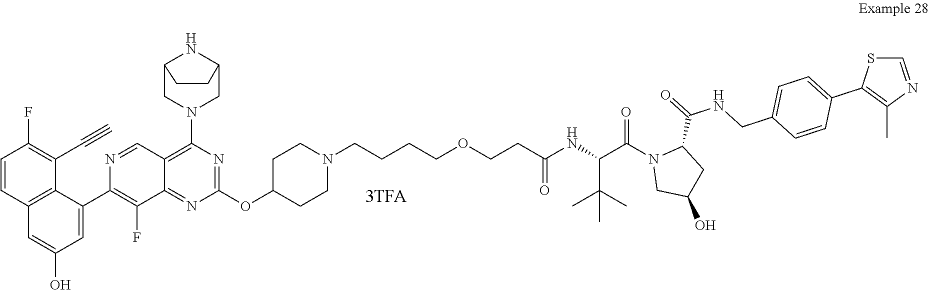 [SMILES: C#Cc1c(F)ccc2cc(O)cc(-c3ncc4c(N5CC6CCC(C5)N6)nc(OC5CCN(CCCCOCCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.09 (s, 1H), 8.97 (s, 1H), 7.92-7.86 (m, 1H), 7.45-7.43 (m, 2H), 7.39-7.37 (m, 3H), 7.35-7.31 (m, 1H), 7.23-7.22 (m, 1H), 5.58-5.41 (m, 1H), 4.86-4.80 (m, 3H), 4.68-4.65 (m, 1H), 4.57-4.53 (m, 1H), 4.49-4.47 (m, 2H), 4.41-4.36 (m, 1H), 4.28 (brs, 2H), 4.01-3.96 (m, 2H), 3.93-3.88 (m, 1H), 3.81-3.78 (m, 1H), 3.70 (brs, 2H), 3.57-3.52 (m, 3H), 3.41 (s, 1H), 3.38-3.35 (m, 0.5H), 3.26-3.18 (m, 2.5H), 2.58-2.49 (m, 3H), 2.48-2.45 (m, 3H), 2.37-2.34 (m, 1H), 2.25-2.04 (m, 8H), 1.90-1.82 (m, 2H), 1.70-1.65 (m, 2H), 1.03-1.01 (m, 9H). m/z (ESI): 1099.5 [M+H]⁺.

### Example 29. Synthesis of Compound 53 Salt

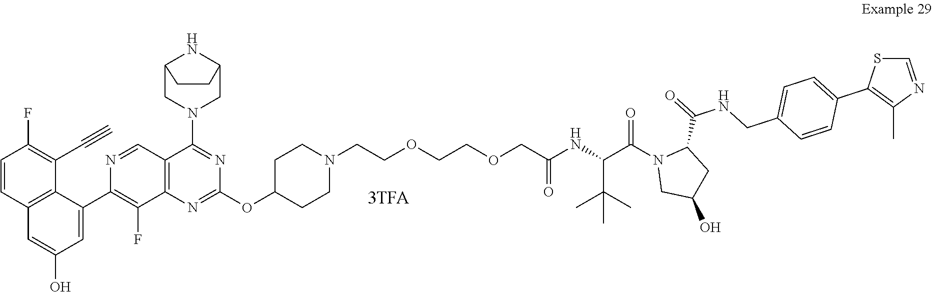 [SMILES: C#Cc1c(F)ccc2cc(O)cc(-c3ncc4c(N5CC6CCC(C5)N6)nc(OC5CCN(CCOCCOCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.08 (s, 1H), 8.92 (s, 1H), 7.88-7.71 (m, 2H), 7.44-7.31 (m, 7H), 7.21 (s, 1H), 5.56-5.44 (m, 1H), 4.83-4.76 (m, 3H), 4.60-4.57 (m, 1H), 4.47-4.38 (m, 3H), 4.27 (brs, 2H), 4.13-4.06 (m, 2H), 3.96-3.85 (m, 4H), 3.80-3.74 (m, 6H), 3.65-3.58 (m, 2H), 3.46-3.37 (m, 3H), 2.46-2.42 (m, 3H), 2.29-2.26 (m, 3H), 2.15-2.04 (m, 6H), 1.53 (s, 1H), 1.05 (s, 9H). m/z (ESI): 1101.3 [M+H]⁺.

### Example 30. Synthesis of Compound 54 Salt

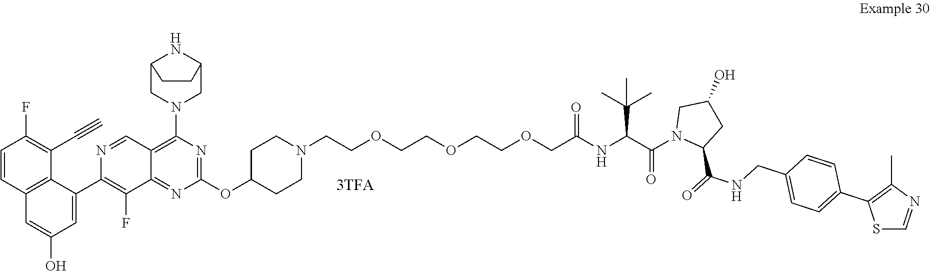 [SMILES: C#Cc1c(F)ccc2cc(O)cc(-c3ncc4c(N5CC6CCC(C5)N6)nc(OC5CCN(CCOCCOCCOCC(=O)N[C@H](C(=O)N6C[C@H](O)C[C@H]6C(=O)NCc6ccc(-c7scnc7C)cc6)C(C)(C)C)CC5)nc4c3F)c12]

Target compound was synthesized according to the procedure of Example 4 with intermediate a as starting material. ¹H NMR (500 MHz, CD₃OD) δ 9.10-9.08 (m, 1H), 8.96 (s, 1H), 7.88 (s, 1H), 7.64-7.63 (m, 1H), 7.45-7.32 (m, 7H), 7.22 (s, 1H), 5.57-5.35 (m, 1H), 4.86-4.79 (m, 3H), 4.69-4.66 (m, 1H), 4.54-4.47 (m, 3H), 4.42-4.38 (m, 1H), 4.28 (brs, 2H), 4.06-4.04 (m, 2H), 3.97-3.78 (m, 7H), 3.72-3.68 (m, 9H), 3.41 (brs, 3H), 2.47 (s, 3H), 2.33-2.04 (m, 10H), 1.03 (s, 9H). m/z (ESI): 1145.3 [M+H]⁺.

### Example 31. Synthesis of Compound 55 Salt

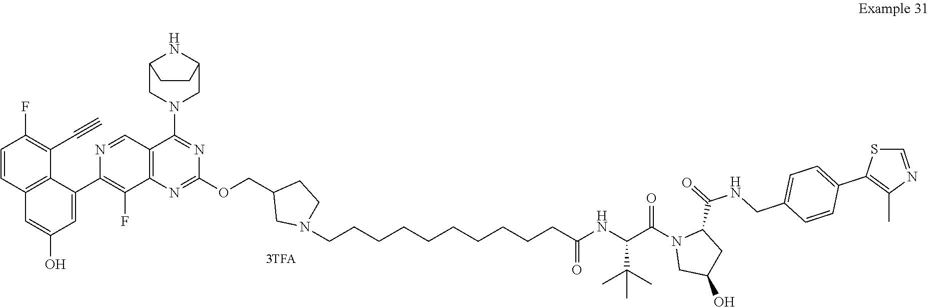 [SMILES: C#Cc1c(F)ccc2cc(O)cc(-c3ncc4c(N5CC6CCC(C5)N6)nc(OCC5CCN(CCCCCCCCCCC(=O)N[C@H](C(=O)N6C[C@H](O)C[C@H]6C(=O)NCc6ccc(-c7scnc7C)cc6)C(C)(C)C)C5)nc4c3F)c12]

Target compound was synthesized according to the procedure of Example 4 with intermediate h as starting material. ¹H NMR (500 MHz, CD₃OD) δ 9.09 (s, 1H), 8.90 (s, 1H), 8.58-8.57 (m, 0.5H), 7.90-7.81 (m, 1.5H), 7.45-7.40 (m, 4H), 7.38-7.33 (m, 2H), 7.21-7.20 (m, 1H), 5.89-5.82 (m, 1H), 5.02-4.99 (m, 1H), 4.82-4.79 (m, 3H), 4.63-4.62 (m, 1H), 4.57-4.54 (m, 1H), 4.43 (s, 1H), 4.28 (brs, 2H), 4.01-3.86 (m, 4H), 3.76-3.74 (m, 1H), 3.55-3.52 (m, 1H), 3.38-3.35 (m, 1H), 3.26-3.25 (m, 2H), 2.48 (s, 3H), 2.31-2.10 (m, 7H), 1.99-1.92 (m, 1H), 1.75 (brs, 2H), 1.62-1.56 (m, 2H), 1.50 (d, J=5 Hz, 3H), 1.39-1.33 (m, 14H), 1.03 (s, 9H). m/z (ESI): 1139.5 [M+H]⁺.

### Example 32. Synthesis of Compound 56 Salt

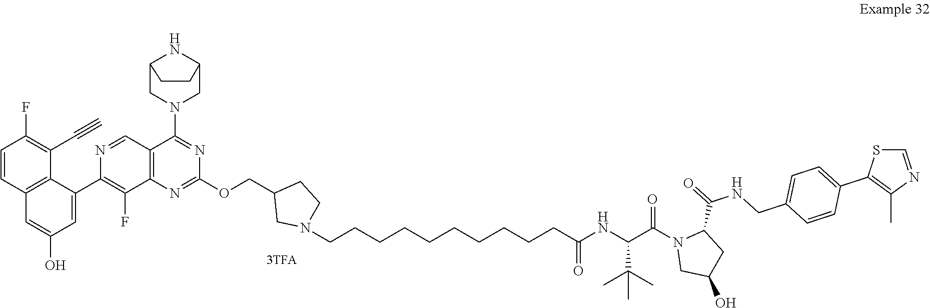 [SMILES: C#Cc1c(F)ccc2cc(O)cc(-c3ncc4c(N5CC6CCC(C5)N6)nc(OCC5CCN(CCCCCCCCCCC(=O)N[C@H](C(=O)N6C[C@H](O)C[C@H]6C(=O)NCc6ccc(-c7scnc7C)cc6)C(C)(C)C)C5)nc4c3F)c12]

Target compound was synthesized according to the procedure of Example 4 with intermediate g as starting material. ¹H NMR (500 MHz, CD₃OD) δ 9.08 (s, 1H), 8.91 (s, 1H), 8.58-8.57 (m, 1H), 7.90-7.81 (m, 2H), 7.45-7.41 (m, 4H), 7.38-7.32 (m, 2H), 7.22 (brs, 1H), 5.01-4.99 (m, 2H), 4.87-4.84 (m, 3H), 4.63-4.50 (m, 4H), 4.43 (s, 1H), 4.29-4.27 (m, 2H), 4.01-3.81 (m, 4H), 3.76-3.73 (m, 2H), 3.37-3.35 (m, 2H), 3.26-3.08 (m, 4H), 2.48 (s, 3H), 2.28-1.93 (m, 10H), 1.74 (brs, 2H), 1.59-1.56 (m, 2H), 1.50 (d, J=10 Hz, 3H), 1.38-1.31 (m, 14H), 1.03 (s, 9H). m/z (ESI): 1153.6 [M+H]⁺.

### Example 33. Synthesis of Compound 57 Salt

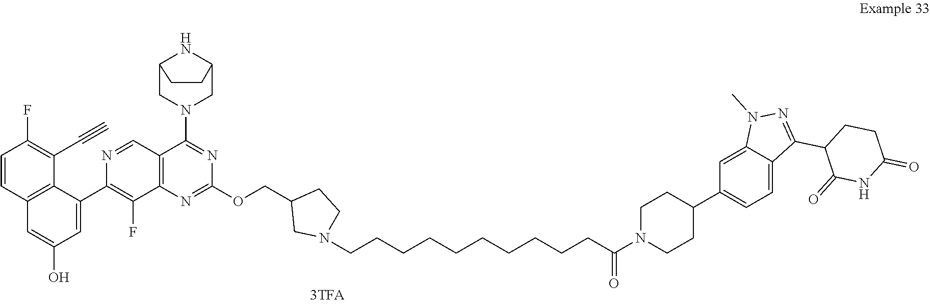 [SMILES: C#Cc1c(F)ccc2cc(O)cc(-c3ncc4c(N5CC6CCC(C5)N6)nc(OCC5CCN(CCCCCCCCCCC(=O)N6CCC(c7ccc8c(C9CCC(=O)NC9=O)nn(C)c8c7)CC6)C5)nc4c3F)c12]

Target compound was synthesized according to the procedure of Example 4 with intermediate g as starting material. ¹H NMR (500 MHz, CD₃OD) δ 10.90 (s, 1H), 9.83-9.36 (m, 1H), 9.13 (s, 2H), 8.00-7.97 (m, 1H), 7.62-7.60 (m, 1H), 7.49-7.41 (m, 3H), 7.18-7.17 (m, 1H), 7.03-7.01 (m, 1H), 4.66-4.31 (m, 6H), 4.21 (brs, 2H), 4.01-3.94 (m, 5H), 3.86-3.47 (m, 10H), 3.14-3.09 (m, 3H), 2.93-2.88 (m, 2H), 2.67-2.54 (m, 4H), 2.36-2.13 (m, 3H), 1.96 (brs, 4H), 1.87-1.78 (m, 1.5H), 1.64-1.47 (m, 5.5H), 1.27 (brs, 12H). m/z (ESI): 1036.7 [M+H]⁺.

### Example 34. Synthesis of Compound 58 Salt

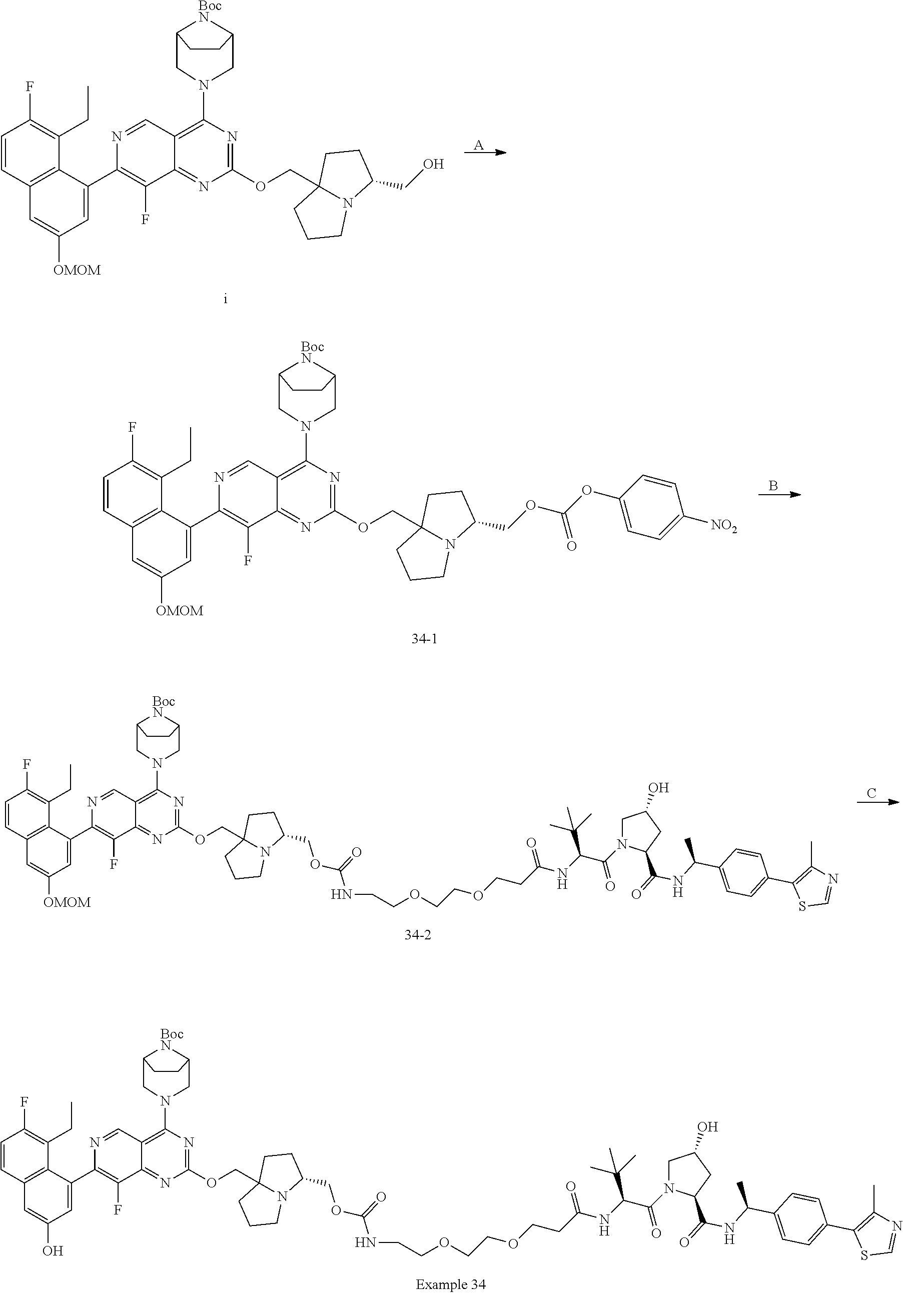

Step A: Et₃N (6.65 mg, 65.71 μmol, 1 eq) was added to a solution of intermediate i (50 mg, 65.71 μmol, 1 eq) in 3 mL of THF, followed by addition of 4-nitrophenyl carbonochloridate (19.87 mg, 98.57 μmol, 1.5 eq). The mixture was stirred at 35° C. for 16 h, then concentrated to afford crude 34-1 that was used in the next step directly. m/z (ESI): 761.5 [M+H]⁺.

Step B: (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (81.73 mg, 127.66 μmol, 2.0 eq) was added to a solution of 34-1 (60 mg, 63.83 μmol, 1 eq) in 3 mL of THF, followed by addition of Et₃N (13 mg, 127.7 μmol, 2 eq). The mixture was stirred at room temperature for 2 h, then concentrated. The residue was purified by column chromatography (MeOH/DCM=1/20) to afford 34-2 (30 mg, 33.80% yield). m/z (ESI): 1390.7 [M+H]⁺.

Step C: 4 M HCl in EA solution (7.87 mg, 215.73 μmol, 10 eq) was added to another solution of 34-2 (30 mg, 21.57 μmol, 1 eq) in 3 mL of DCM. The mixture was stirred at room temperature for 10 min and concentrated. The residue was purified by PREP-HPLC (0.05% NH₃·H₂O/MeCN) to afford white solid (3.8 mg, 13.75% yield). ¹H NMR (500 MHz, CD₃OD) δ 9.07 (s, 1H), 8.87 (s, 1H), 7.69-7.66 (m, 1H), 7.43-7.38 (m, 4H), 7.31-7.30 (m, 1H), 7.26-7.23 (m, 1H), 7.06-7.05 (m, 1H), 5.01-4.97 (m, 1H), 4.64-4.55 (m, 4H), 4.42-4.38 (m, 2H), 4.30-4.29 (m, 3H), 3.87-3.85 (m, 1H), 3.75-3.58 (m, 12H), 3.54-3.52 (m, 2H), 3.35 (s, 1H), 3.01 (brs, 1H), 2.90 (brs, 1H), 2.59-2.54 (m, 1H), 2.49-2.45 (m, 5H), 2.26-2.17 (m, 3H), 2.06-2.02 (m, 1H), 1.96-1.73 (m, 10H), 1.57-1.49 (m, 3H), 1.32-1.29 (m, 3H), 1.03 (s, 9H), 0.81-0.77 (m, 3H). m/z (ESI): 1246.4 [M+H]⁺.

### Example 35. Synthesis of Compound 59 Salt

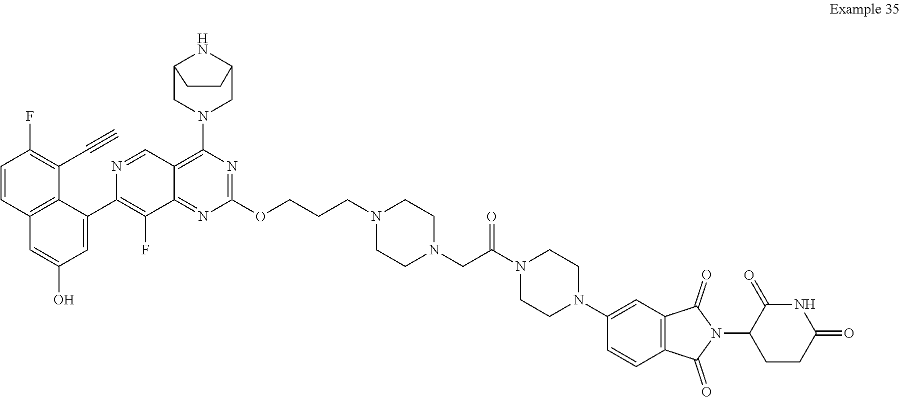 [SMILES: C#Cc1c(F)ccc2cc(O)cc(-c3ncc4c(N5CC6CCC(C5)N6)nc(OCCCN5CCN(CC(=O)N6CCN(c7ccc8c(c7)C(=O)N(C7CCC(=O)NC7=O)C8=O)CC6)CC5)nc4c3F)c12]

Target compound was synthesized according to the procedure of Example 4. ¹H NMR (500 MHz, Methanol-d6) δ 1.31-1.34 (m, 2H), 1.85-1.93 (m, 4H), 2.06-2.14 (m, 4H), 2.65-2.75 (m, 14H), 3.40 (s, 1H), 3.51-3.55 (m, 4H), 3.76-3.78 (m, 7H), 4.54-4.58 (m, 2H), 4.61-4.69 (m, 3H), 5.11 (dd, J=12.5, 5.4 Hz, 1H), 7.24 (d, J=2.1 Hz, 1H), 7.25-7.29 (m, 1H), 7.34 (d, J=8.9 Hz, 1H), 7.38-7.41 (m, 2H), 7.72 (d, J=8.5 Hz, 1H), 7.89 (dd, J=9.0, 5.8 Hz, 1H), 9.03 (s, 1H). m/z (ESI): 968.29 [M+H]⁺.

### Example 36. Synthesis of Compound 60 Salt

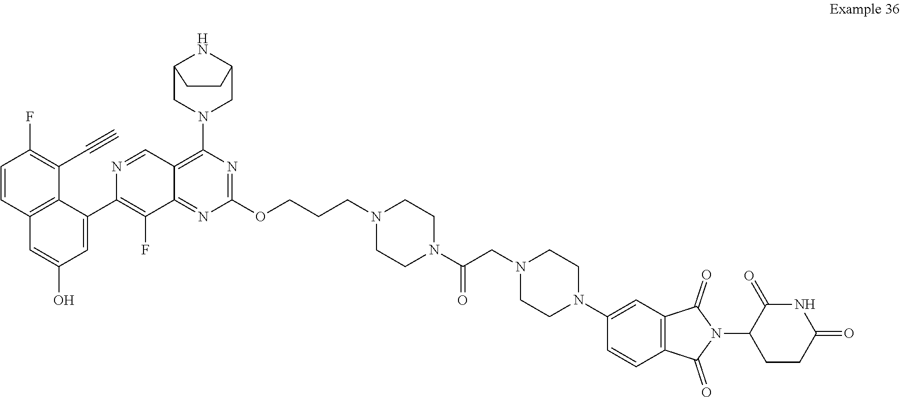 [SMILES: C#Cc1c(F)ccc2cc(O)cc(-c3ncc4c(N5CC6CCC(C5)N6)nc(OCCCN5CCN(C(=O)CN6CCN(c7ccc8c(c7)C(=O)N(C7CCC(=O)NC7=O)C8=O)CC6)CC5)nc4c3F)c12]

Target compound was synthesized according to the procedure of Example 4. ¹H NMR (500 MHz, DMSO) δ 1.66 (s, 6H), 1.92-1.93 (m, 3H), 2.00-2.07 (m, 1H), 2.33-2.49 (m, 12H), 2.54-2.62 (m, 2H), 3.21 (s, 4H), 3.57-3.67 (m, 7H), 3.95 (s, 1H), 4.33 (d, J=12.0 Hz, 1H), 4.41 (s, 3H), 4.47 (d, J=12.5 Hz, 1H), 5.09 (dd, J=12.5, 5.0 Hz, 1H), 7.19 (s, 1H), 7.25 (d, J=8.0 Hz, 1H), 7.36 (s, 1H), 7.41 (s, 1H), 7.48 (t, J=9.0 Hz, 1H), 7.69 (d, J=8.5 Hz, 1H), 7.99 (dd, J=8.5, 6.0 Hz, 1H), 9.04 (s, 1H), 11.08 (s, 1H). m/z (ESI): 968.3 [M+H]⁺.

### Example 37. Synthesis of Compound 61 Salt

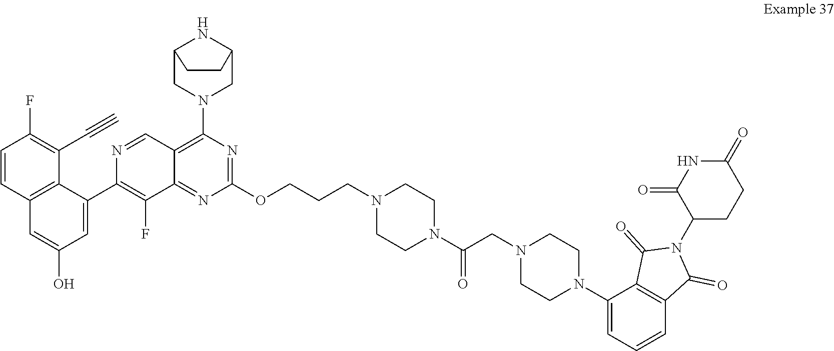 [SMILES: C#Cc1c(F)ccc2cc(O)cc(-c3ncc4c(N5CC6CCC(C5)N6)nc(OCCCN5CCN(C(=O)CN6CCN(c7cccc8c7C(=O)N(C7CCC(=O)NC7=O)C8=O)CC6)CC5)nc4c3F)c12]

Target compound was synthesized according to the procedure of Example 4. ¹H NMR (500 MHz, Methanol-d4) δ 2.11 (s, 4H), 2.15-2.28 (m, 4H), 2.30-2.38 (m, 1H), 2.41-2.51 (m, 1H), 2.72-2.86 (m, 2H), 3.01-3.10 (m, 1H), 3.13-3.25 (m, 3H), 3.41 (s, 1H), 3.49 (t, J=13.1 Hz, 1H), 3.80 (d, J=6.8 Hz, 3H), 3.86-3.99 (m, 3H), 4.06-4.14 (m, 1H), 4.19 (s, 2H), 4.33-4.46 (m, 2H), 4.70-4.84 (m, 3H), 6.98 (d, J=8.6 Hz, 1H), 7.11-7.19 (m, 1H), 7.19-7.24 (m, 2H), 7.34 (d, J=10.8, 2.6 Hz, 1H), 7.56 (d, J=8.4 Hz, 1H), 7.70-7.82 (m, 1H), 9.10 (s, 1H). m/z (ESI): 968.4 [M+H]⁺.

### Example 38. Synthesis of Compound 62 Salt

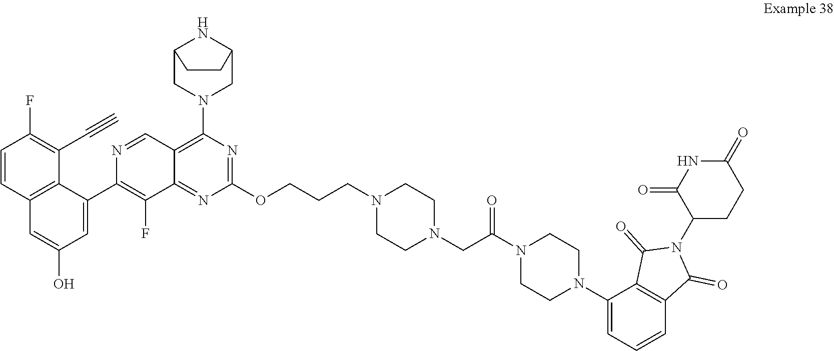 [SMILES: C#Cc1c(F)ccc2cc(O)cc(-c3ncc4c(N5CC6CCC(C5)N6)nc(OCCCN5CCN(CC(=O)N6CCN(c7cccc8c7C(=O)N(C7CCC(=O)NC7=O)C8=O)CC6)CC5)nc4c3F)c12]

Target compound was synthesized according to the procedure of Example 4. ¹H NMR (500 MHz, DMSO-d6) δ 1.66 (s, 6H), 1.92-1.93 (m, 3H), 2.00-2.07 (m, 1H), 2.33-2.49 (m, 12H), 2.54-2.62 (m, 2H), 3.21 (s, 4H), 3.57-3.67 (m, 7H), 3.95 (s, 1H), 4.33 (d, J=12.0 Hz, 1H), 4.41 (s, 3H), 4.47 (d, J=12.5 Hz, 1H), 5.09 (dd, J=12.5, 5.0 Hz, 1H), 7.19 (s, 1H), 7.25 (d, J=8.0 Hz, 1H), 7.36 (s, 1H), 7.41 (s, 1H), 7.48 (t, J 34-=9.0 Hz, 1H), 7.69 (d, J=8.5 Hz, 1H), 7.99 (dd, J=8.5, 6.0 Hz, 1H), 9.04 (s, 1H), 11.08 (s, 1H). m/z (ESI): 968.4 [M+H]⁺.

### Example 39. Synthesis of Compound 63 Salt

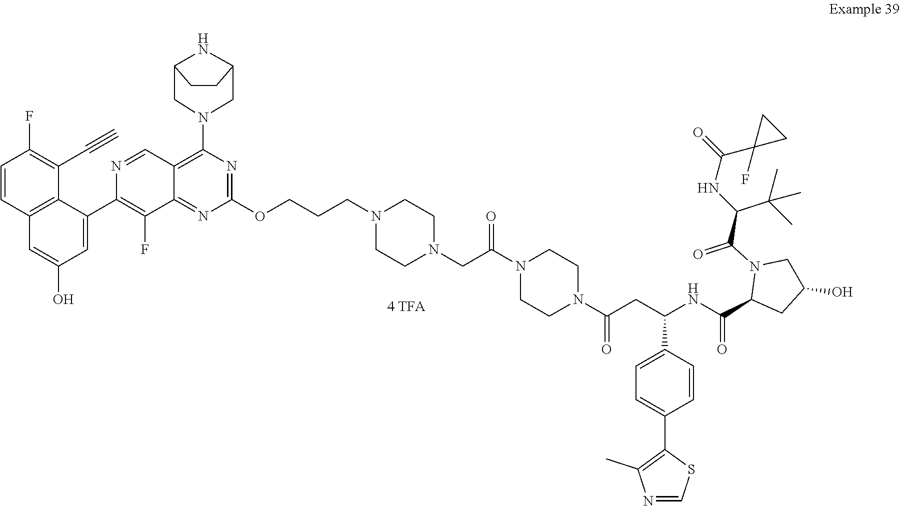 [SMILES: C#Cc1c(F)ccc2cc(O)cc(-c3ncc4c(N5CC6CCC(C5)N6)nc(OCCCN5CCN(CC(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)CC5)nc4c3F)c12]

Target compound was synthesized according to the procedure of Example 4. ¹H NMR (500 MHz, Methanol-d4) δ 1.06 (s, 9H), 1.37-1.30 (m, 6H), 2.04-1.96 (m, 2H), 2.15-2.12 (m, 6H), 2.28-2.25 (m, 3H), 2.47 (s, 3H), 3.06-2.99 (m, 3H), 3.38 (s, 2H), 3.61-3.51 (m, 10H), 3.78-3.76 (m, 1H), 3.85-3.82 (m, 1H), 3.98-3.91 (m, 2H), 4.29-4.27 (m, 2H), 4.45 (brs, 1H), 4.64-4.55 (m, 4H), 4.74-4.73 (m, 1H), 4.83-4.81 (m, 4H), 7.21-7.20 (m, 1H), 7.38-7.33 (m, 2H), 7.50-7.45 (m, 4H), 7.90-7.87 (m, 1H), 8.91 (s, 1H), 9.07 (s, 1H). m/z (ESI): 1268.7 [M+H]⁺.

### Example 40. Synthesis of Compound 64 Salt

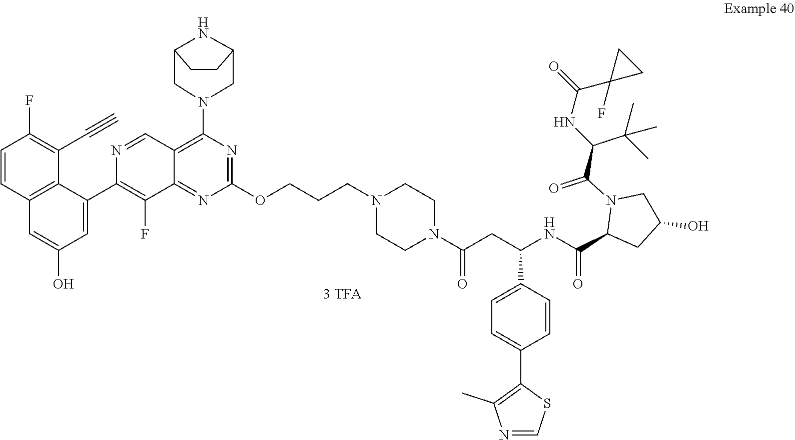 [SMILES: C#Cc1c(F)ccc2cc(O)cc(-c3ncc4c(N5CC6CCC(C5)N6)nc(OCCCN5CCN(C(=O)C[C@H](NC(=O)[C@@H]6C[C@@H](O)CN6C(=O)[C@@H](NC(=O)C6(F)CC6)C(C)(C)C)c6ccc(-c7scnc7C)cc6)CC5)nc4c3F)c12]

Target compound was synthesized according to the procedure of Example 4. ¹H NMR (500 MHz, Methanol-d4) δ1.05 (s, 9H), 1.35-1.26 (m, 4H), 1.98-1.93 (m, 1H), 2.22-2.1 (m, 5H), 2.32-2.30 (m, 2H), 2.49 (s, 3H), 3.00-2.91 (m, 1H), 3.26-3.10 (m, 1H), 3.42-3.41 (m, 3H), 3.67-3.56 (m, 1H), 3.78-3.75 (m, 1H), 3.85-3.82 (m, 1H), 4.02-3.94 (m, 2H), 4.29-4.27 (m, 2H), 4.45 (brs, 1H), 4.61-4.54 (m, 3H), 4.74-4.72 (m, 1H), 4.84-4.79 (m, 2H), 5.43-5.32 (m, 1H), 7.23-7.22 (m, 1H), 7.38-7.32 (m, 2H), 7.51-7.42 (m, 5H), 7.90-7.87 (m, 1H), 8.96 (s, 1H), 9.10 (s, 1H). m/z (ESI): 1142.5 [M+H]⁺.

### Example 41. Synthesis of Compound 65 Salt

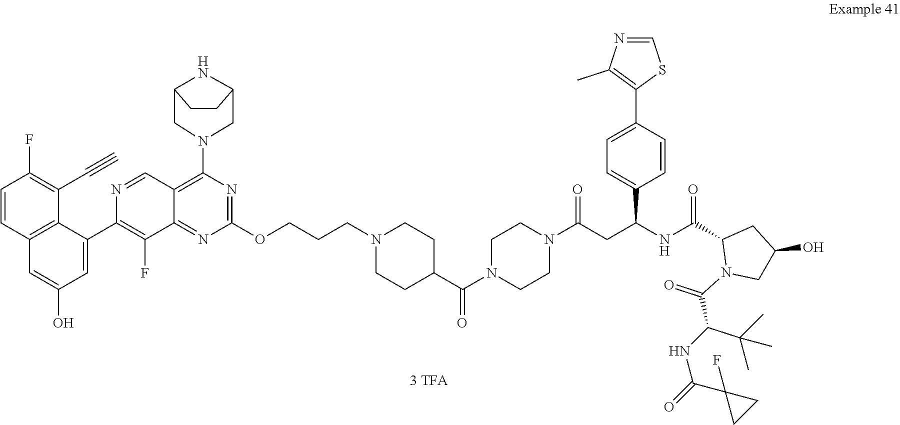 [SMILES: C#Cc1c(F)ccc2cc(O)cc(-c3ncc4c(N5CC6CCC(C5)N6)nc(OCCCN5CCC(C(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)CC5)nc4c3F)c12]

Target compound was synthesized according to the procedure of Example 4. ¹H NMR (500 MHz, Methanol-d4) δ1.05 (s, 9H), 1.35-1.26 (m, 4H), 1.98-1.93 (m, 1H), 2.22-2.1 (m, 5H), 2.32-2.30 (m, 2H), 2.49 (s, 3H), 3.00-2.91 (m, 1H), 3.26-3.10 (m, 1H), 3.42-3.41 (m, 3H), 3.67-3.56 (m, 1H), 3.78-3.75 (m, 1H), 3.85-3.82 (m, 1H), 4.02-3.94 (m, 2H), 4.29-4.27 (m, 2H), 4.45 (brs, 1H), 4.61-4.54 (m, 3H), 4.74-4.72 (m, 1H), 4.84-4.79 (m, 2H), 5.43-5.32 (m, 1H), 7.23-7.22 (m, 1H), 7.38-7.32 (m, 2H), 7.51-7.42 (m, 5H), 7.90-7.87 (m, 1H), 8.96 (s, 1H), 9.10 (s, 1H). m/z (ESI): 1142.5 [M+H]⁺.

### Example 42. Synthesis of Compound 66

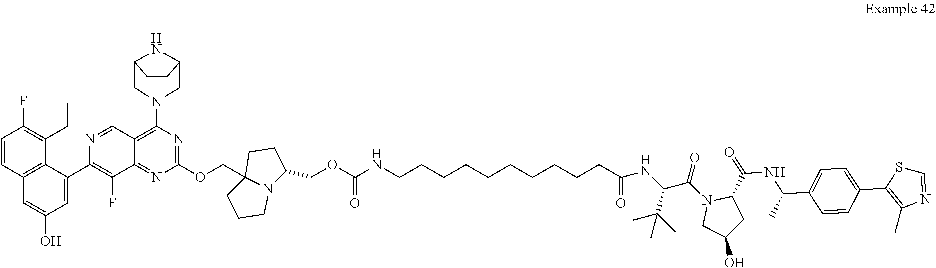 [SMILES: CCc1c(F)ccc2cc(O)cc(-c3ncc4c(N5CC6CCC(C5)N6)nc(OCC56CCCN5[C@@H](COC(=O)NCCCCCCCCCCC(=O)N[C@H](C(=O)N5C[C@H](O)C[C@H]5C(=O)N[C@@H](C)c5ccc(-c7scnc7C)cc5)C(C)(C)C)CC6)nc4c3F)c12]

Target compound was synthesized according to the procedure of Example 4. ¹H NMR (500 MHz, Methanol-d4) δ 1.03 (s, 9H), 1.50-4.49 (m, 3H), 2.04-1.56 (m, 16H), 2.20-2.15 (m, 3H), 2.33-2.24 (m, 2H), 2.50-2.47 (m, 4H), 2.88-2.84 (m, 1H), 3.00-2.94 (m, 1H), 3.15-3.10 (m, 2H), 3.57-3.52 (m, 1H), 3.76-3.65 (m, 5H), 3.88-3.86 (m, 1H), 4.30-4.27 (m, 3H), 4.42-4.36 (m, 2H), 4.63-4.54 (m, 5H), 5.01-4.97 (m, 1H), 7.06-7.05 (m, 1H), 7.26-7.22 (m, 1H), 7.30-7.29 (m, 1H), 7.43-7.38 (m, 4H), 7.68-7.65 (m, 1H), 8.87 (s, 1H), 9.06 (s, 1H). m/z (ESI): 1186.5 [M+H]⁺.

### Example 43. Synthesis of Compound 67

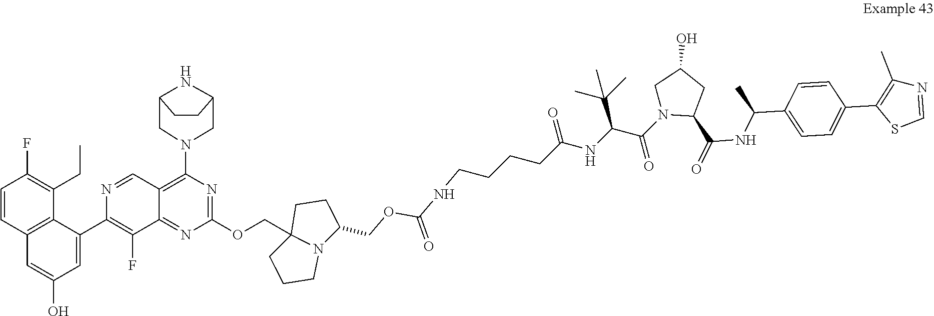 [SMILES: CCc1c(F)ccc2cc(O)cc(-c3ncc4c(N5CC6CCC(C5)N6)nc(OCC56CCCN5[C@@H](COC(=O)NCCCCC(=O)N[C@H](C(=O)N5C[C@H](O)C[C@H]5C(=O)N[C@@H](C)c5ccc(-c7scnc7C)cc5)C(C)(C)C)CC6)nc4c3F)c12]

Target compound was synthesized according to the procedure of Example 4. ¹H NMR (500 MHz, Methanol-d4) δ 1.03 (s, 9H), 1.50-4.49 (m, 3H), 2.04-1.56 (m, 16H), 2.20-2.15 (m, 3H), 2.33-2.24 (m, 2H), 2.50-2.47 (m, 4H), 2.88-2.84 (m, 1H), 3.00-2.94 (m, 1H), 3.15-3.10 (m, 2H), 3.57-3.52 (m, 1H), 3.76-3.65 (m, 5H), 3.88-3.86 (m, 1H), 4.30-4.27 (m, 3H), 4.42-4.36 (m, 2H), 4.63-4.54 (m, 5H), 5.01-4.97 (m, 1H), 7.06-7.05 (m, 1H), 7.26-7.22 (m, 1H), 7.30-7.29 (m, 1H), 7.43-7.38 (m, 4H), 7.68-7.65 (m, 1H), 8.87 (s, 1H), 9.06 (s, 1H). m/z (ESI): 1186.5 [M+H]⁺.

### Example 44. Synthesis of Compound 68

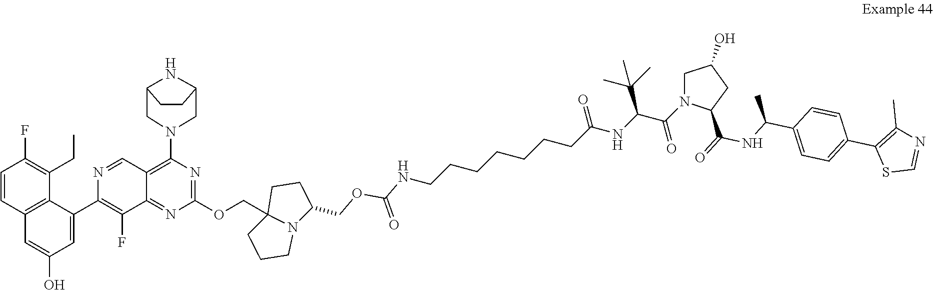 [SMILES: CCc1c(F)ccc2cc(O)cc(-c3ncc4c(N5CC6CCC(C5)N6)nc(OCC56CCCN5[C@@H](COC(=O)NCCCCCCCC(=O)N[C@H](C(=O)N5C[C@H](O)C[C@H]5C(=O)N[C@@H](C)c5ccc(-c7scnc7C)cc5)C(C)(C)C)CC6)nc4c3F)c12]

Target compound was synthesized according to the procedure of Example 4. ¹H NMR (500 MHz, Methanol-d4) δ 0.81-0.78 (m, 3H), 1.03 (s, 9H), 1.33-1.30 (m, 8H), 1.51-1.47 (m, 5H), 2.04-1.56 (m, 16H), 2.31-2.15 (m, 5H), 2.51-2.47 (m, 4H), 2.83-2.81 (m, 1H), 2.94-2.91 (m, 1H), 3.11-3.08 (m, 2H), 3.49-3.45 (m, 1H), 3.65 (brs, 2H), 3.75-3.72 (m, 3H), 3.89-3.86 (m, 1H), 4.27-4.25 (m, 3H), 4.36-4.34 (m, 1H), 4.42 (brs, 1H), 4.62-4.55 (m, 5H), 5.02-4.98 (m, 1H), 7.06-7.05 (m, 1H), 7.26-7.22 (m, 1H), 7.30-7.29 (m, 1H), 7.44-7.40 (m, 4H), 7.68-7.65 (m, 1H), 8.87 (s, 1H), 9.06 (s, 1H). m/z (ESI): 1128.5 [M+H]⁺.

### Example 45. Synthesis of Compound 69 Salt

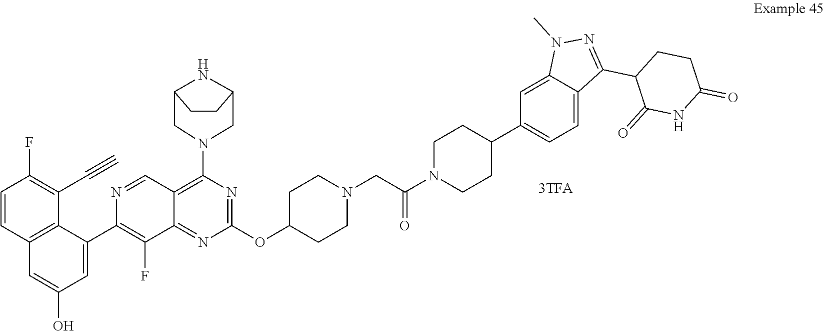 [SMILES: C#Cc1c(F)ccc2cc(O)cc(-c3ncc4c(N5CC6CCC(C5)N6)nc(OC5CCN(CC(=O)N6CCC(c7ccc8c(C9CCC(=O)NC9=O)nn(C)c8c7)CC6)CC5)nc4c3F)c12]

Target compound was synthesized according to the procedure of Example 1. ¹H NMR (500 MHz, Methanol-d4) δ 9.08 (s, 1H), 7.88 (dd, J=9.2, 5.7 Hz, 1H), 7.66 (d, J=8.4 Hz, 1H), 7.44-7.28 (m, 3H), 7.21 (d, J=2.5 Hz, 1H), 7.09 (d, J=8.5 Hz, 1H), 4.85-4.77 (m, 3H), 4.71 (d, J=13.3 Hz, 1H), 4.47-4.23 (m, 5H), 4.00 (s, 3H), 3.96 (d, J=14.0 Hz, 2H), 3.89-3.43 (m, 4H), 3.37 (s, 1H), 3.03 (t, J=12.4 Hz, 1H), 2.88 (t, J=12.8 Hz, 1H), 2.83-2.66 (m, 2H), 2.59-2.26 (m, 6H), 2.23-2.09 (m, 5H), 2.01 (d, J=13.3 Hz, 2H), 1.91-1.67 (m, 2H). m/z (ESI): 909.5 [M+H]⁺.

### Example 46. Synthesis of Compound 70 Salt

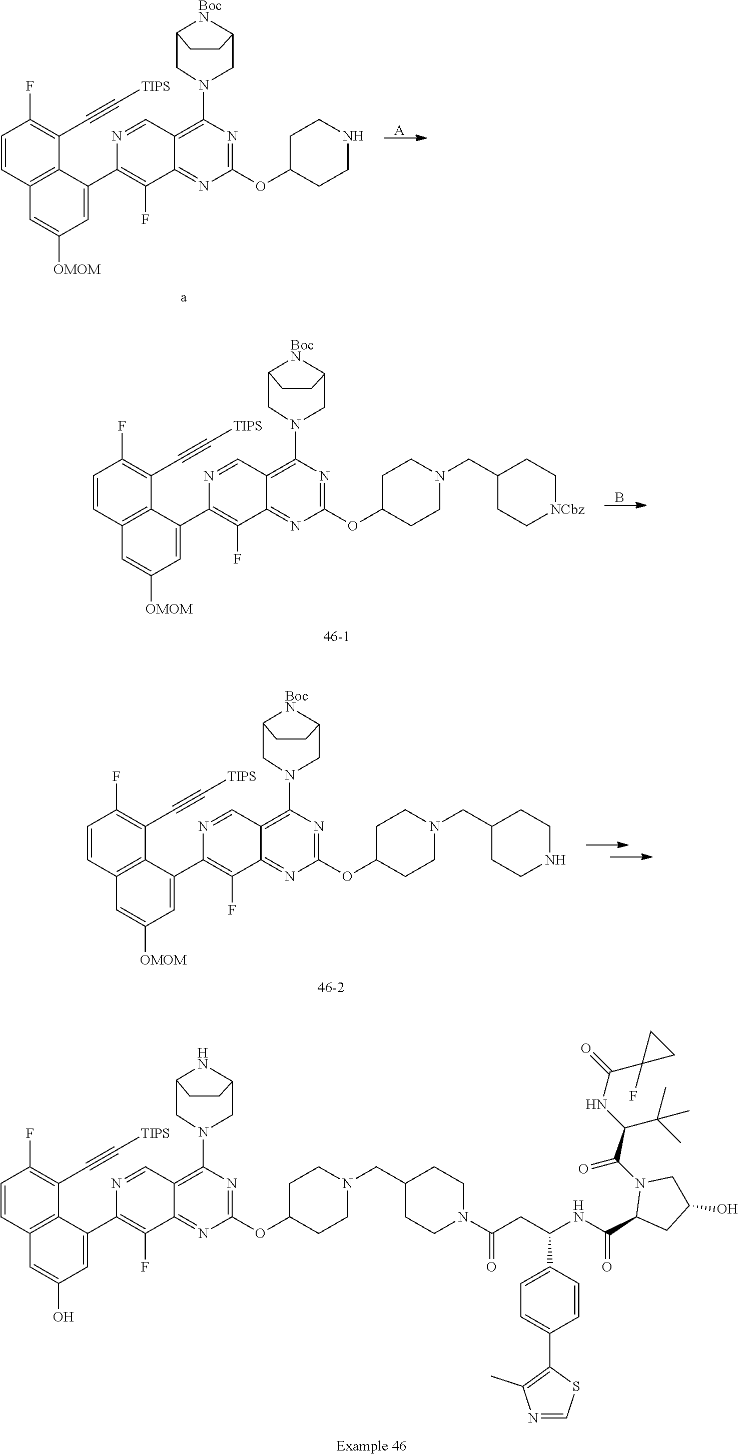

Step A: benzyl 4-formylpiperidine-1-carboxylate (35.20 mg, 142.33 μmol, 1.2 eq) was added to a solution of intermediate a (100 mg, 118.61 μmol, 1 eq) in 5 mL of DCE, followed by addition of sodium triacetoxyborohyride (30.17 mg, 166 μmol, 1.4 eq). The mixture was stirred at room temperature overnight, then concentrated. The residue was purified by column chromatography (MeOH/DCM=1/9) to afford yellow solid 46-1 (100 mg, yield 78%), m/z (ESI): 1074.5 [M+H]⁺.

Step B: 50 mg of Pd/C was added to a solution of 46-1 (100 mg, 93.08 μmol, 1 eq) in 5 mL of MeOH. The mixture was stirred at room temperature for 12 h under hydrogen atmosphere, then filtered. The filtrated was concentrated to afford yellow solid. (0.08 g, yield 92%). m/z (ESI): 940.5 [M+H]⁺.

Target compound was synthesized according to the procedure of Example 1. ¹H NMR (500 MHz, Methanol-d4) δ 9.08 (s, 1H), 8.92 (s, 1H), 7.88 (dd, J=9.2, 5.7 Hz, 2H), 7.47 (d, J=4.1 Hz, 4H), 7.39-7.31 (m, 2H), 7.21 (d, J=2.5 Hz, 1H), 5.36 (q, J=6.6 Hz, 1H), 4.80 (d, J=12.4 Hz, 2H), 4.73 (d, J=9.2 Hz, 1H), 4.56 (t, J=8.4 Hz, 2H), 4.47 (d, J=28.7 Hz, 3H), 4.28 (s, 2H), 4.07 (s, 1H), 4.01-3.90 (m, 2H), 3.86-3.74 (m, 2H), 3.73-3.48 (m, 2H), 3.37 (s, 2H), 3.24-2.95 (m, 6H), 2.63 (q, J=13.9 Hz, 1H), 2.49 (s, 3H), 2.39-2.07 (m, 11H), 1.96 (ddd, J=13.4, 9.4, 4.4 Hz, 1H), 1.81 (d, J=14.1 Hz, 3H), 1.31 (dd, J=17.7, 8.0 Hz, 4H), 1.05 (s, 9H). m/z (ESI): 1196.5 [M+H]⁺.

### Example 47. Synthesis of Compound 71 Salt

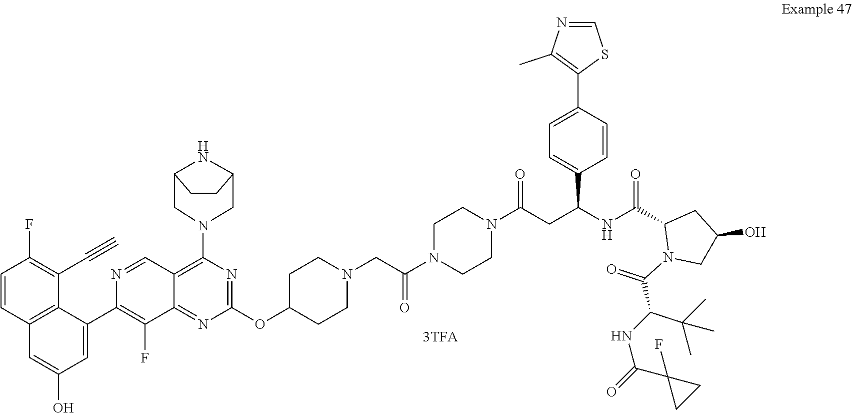 [SMILES: C#Cc1c(F)ccc2cc(O)cc(-c3ncc4c(N5CC6CCC(C5)N6)nc(OC5CCN(CC(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)CC5)nc4c3F)c12]

Target compound was synthesized according to the procedure of Example 46. ¹H NMR (500 MHz, Methanol-d4) δ 9.04 (s, 1H), 8.88 (s, 1H), 7.84 (dd, J=9.2, 5.7 Hz, 1H), 7.48-7.39 (m, 4H), 7.38-7.25 (m, 2H), 7.17 (d, J=2.5 Hz, 1H), 4.83-4.66 (m, 3H), 4.57-4.48 (m, 1H), 4.41 (s, 1H), 4.27 (d, J=26.2 Hz, 4H), 3.92 (s, 2H), 3.82-3.68 (m, 2H), 3.66-3.49 (m, 8H), 3.37 (d, J=28.9 Hz, 6H), 3.15-2.96 (m, 2H), 2.44 (s, 3H), 2.38-2.23 (m, 3H), 2.12 (dq, J=23.6, 12.0, 11.6 Hz, 6H), 2.01-1.86 (m, 1H), 1.38-1.18 (m, 4H), 1.01 (s, 9H). m/z (ESI): 909.5 [M+H]⁺.

### Example 48. Synthesis of Compound 72 Salt

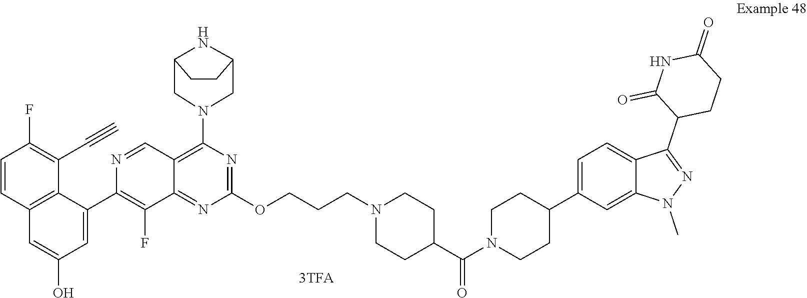 [SMILES: C#Cc1c(F)ccc2cc(O)cc(-c3ncc4c(N5CC6CCC(C5)N6)nc(OCCCN5CCC(C(=O)N6CCC(c7ccc8c(C9CCC(=O)NC9=O)nn(C)c8c7)CC6)CC5)nc4c3F)c12]

Target compound was synthesized according to the procedure of Example 4. 1H NMR (500 MHz, Methanol-d4) δ 9.08 (s, 1H), 7.91-7.84 (m, 1H), 7.66 (d, J=8.3 Hz, 1H), 7.40-7.30 (m, 3H), 7.22 (s, 1H), 7.09 (d, J=8.3 Hz, 1H), 4.81 (m, 3H), 4.72 (d, J=12.5 Hz, 1H), 4.62 (m, 2H), 4.36 (dd, J=9.1, 5.0 Hz, 1H), 4.28 (m, 2H), 4.21 (m, 1H), 4.00 (s, 3H), 3.99-3.90 (m, 2H), 3.74 (m, 2H), 3.38 (s, 2H), 3.27 (m, 1H), 3.12 (m, 3H), 3.03 (m, 1H), 2.82-2.68 (m, 3H), 2.46 (m, 1H), 2.32 (m, 3H), 2.13 (m, 4H), 2.10-1.95 (m, 6H), 1.72 (m, 2H). m/z (ESI): 937.5 [M+H]⁺.

### Example 49. Synthesis of Compound 73 Salt

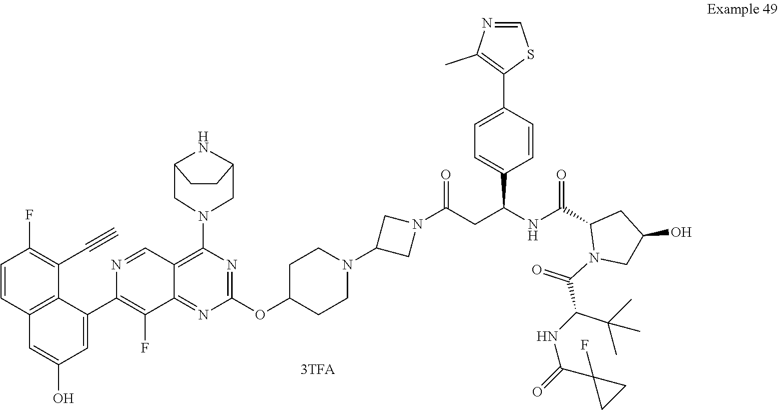 [SMILES: C#Cc1c(F)ccc2cc(O)cc(-c3ncc4c(N5CC6CCC(C5)N6)nc(OC5CCN(C6CN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)C6)CC5)nc4c3F)c12]

Target compound was synthesized according to the procedure of Example 46. ¹H NMR (500 MHz, Methanol-d4) δ 9.05 (s, 1H), 8.89 (s, 1H), 7.84 (dd, J=9.2, 5.7 Hz, 1H), 7.44 (t, J=4.4 Hz, 4H), 7.40-7.25 (m, 2H), 7.17 (s, 1H), 4.82-4.60 (m, 4H), 4.56-4.31 (m, 4H), 4.29-4.06 (m, 4H), 3.91 (t, J=13.0 Hz, 2H), 3.85-3.65 (m, 2H), 3.57-3.31 (m, 4H), 2.92-2.62 (m, 2H), 2.44 (d, J=2.1 Hz, 3H), 2.39-2.22 (m, 3H), 2.21-2.03 (m, 7H), 2.00-1.84 (m, 1H), 1.28 (dd, J=33.9, 13.6 Hz, 5H), 1.03 (s, 9H). m/z (ESI): 1196.5 [M+H]⁺.

### Example 50. Synthesis of Compound 74 Salt

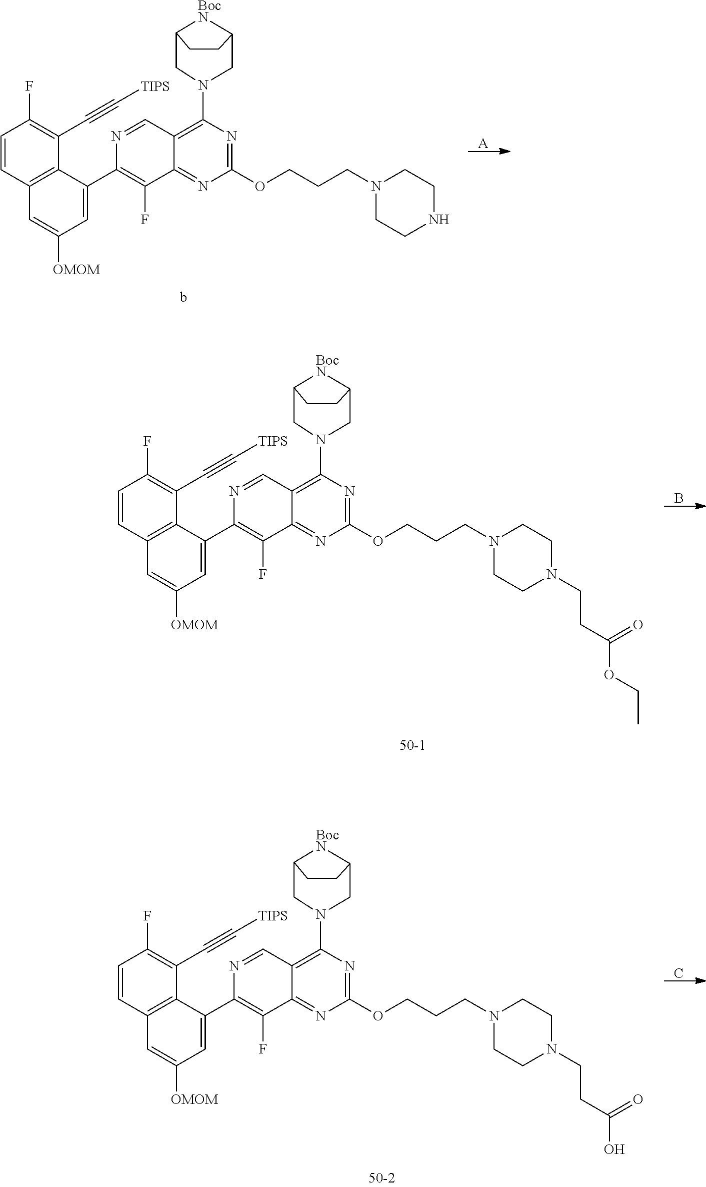

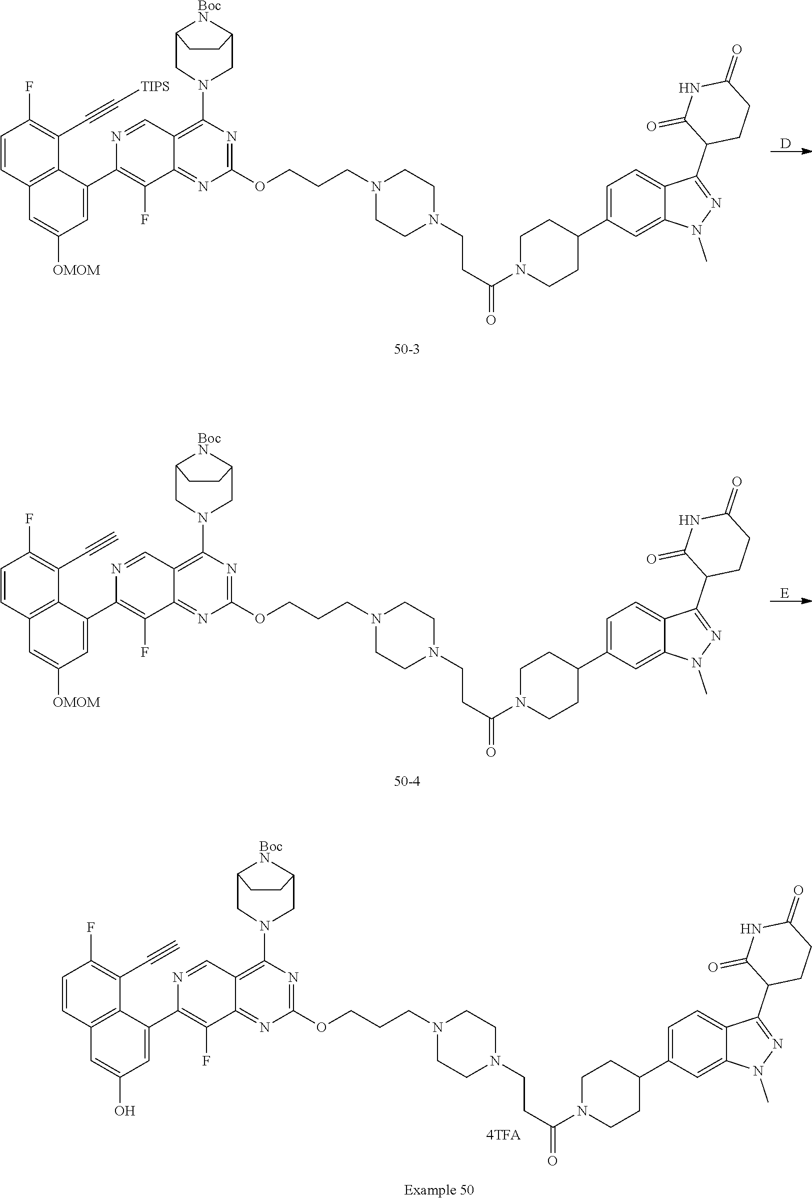

Step A: DBU (171.80 mg, 1.13 mmol, 168.43 μL, 5 eq) was added to a solution of intermediate b (200 mg, 225.69 μmol, 1 eq) in 5 mL of acetronitrile, followed by addition of ethyl acrylate (27.11 mg, 270.83 μmol, 1.2 eq). The mixture was stirred at 20° C. overnight, then treated with water and EtOAc and stirred for 10 min. Separated organic layer was concentrated. The residue was purified by column chromatography (MeOH/DCM=1/9) to afford yellow oil 50-1 (140 mg, 62% yield). m/z (ESI): 987 [M+H]⁺.

Step B: LiOH (17 mg, 709.75 umol, 5 eq) was added to a solution of 50-1 (4 mL) in 2 mL of water. The mixture was stirred at 50° C. for 1 h, then cooled to room temperature and concentrated. The pH of residue was adjusted to 4-5 and extracted with EA. The combined organic layer was washed with brine, dried over Na₂SO₄ and filtered. The filtrate was concentrated to afford yellow solid 50-2 (110 mg, 114.80 umol, 80.87% yield). m/z (ESI): 959 [M+H]⁺.

Step C: 3-(1-methyl-6-(piperidin-4-yl)-1H-indazol-3-yl)piperidine-2,6-dione (44.9 mg, 1387.7 umol, 1.2 eq) was added to a solution of 50-2 (110 mg, 114.80 umol, 1 eq) in 5 mL of DMF, followed by addition of DIEA (59.35 mg, 4 eq) and HATU (47.6 mg, 126.28 umol). The mixture was stirred at room temperature for 0.5 h, then concentrated. The residue was purified by column chromatography (MeOH/DCM=1/9) to afford 50-3 (105 mg, 82.9 umol, 72.210% yield). m/z (ESI): 1267 [M+H]⁺.

Step D: TBAF (1 mL) was added to a solution of 50-3 (105 mg, 82.9 umol, 1 eq) in 2 mL of THF. The mixture was stirred at room temperature for 1 h, then concentrated. The residue was purified by column chromatography (MeOH/DCM=1/9) to afford 50-4 (70 mg, 63.05 umol, 76.05 yield). m/z (ESI): 1111 [M+H]⁺.

Step E: HCl in EtOAc (1 mL) was added to a solution of 50-4 (70 mg, 63.05 umol, 1 eq) in 2 mL of DCM. The mixture was concentrated, and the residue was purified by Prep-HPLC to afford yellow solid (15 mg, 16.7% yield). ¹H NMR (500 MHz, Methanol-d4) δ 1.74 (dd, J=44.7, 11.2 Hz, 3H), 1.97 (t, J=13.1 Hz, 3H), 2.09-2.20 (m, 5H), 2.22 (d, J=7.8 Hz, 3H), 2.31 (dd, J=13.3, 5.9 Hz, 1H), 2.45 (dd, J=9.2, 4.9 Hz, 1H), 2.77 (q, J=16.5, 14.3 Hz, 4H), 2.89 (s, 3H), 3.13 (s, 3H), 3.40 (s, 2H), 3.94 (dd, J=21.9, 14.0 Hz, 3H), 4.00 (s, 4H), 4.06 (d, J=13.6 Hz, 1H), 4.28 (d, J=10.7 Hz, 2H), 4.36 (dd, J=9.3, 5.1 Hz, 1H), 4.62 (q, J=6.3 Hz, 2H), 4.71 (d, J=13.2 Hz, 1H), 4.82 (d, J=14.1 Hz, 3H), 7.08 (d, J=8.5 Hz, 1H), 7.22 (d, J=2.6 Hz, 1H), 7.35 (td, J=10.3, 9.7, 5.1 Hz, 3H), 7.65 (d, J=8.4 Hz, 1H), 7.88 (d, J=8.8 Hz, 1H), 9.07 (s, 1H). m/z (ESI): 967 [M+H]⁺.

### Example 51. Synthesis of Compound 75 Salt

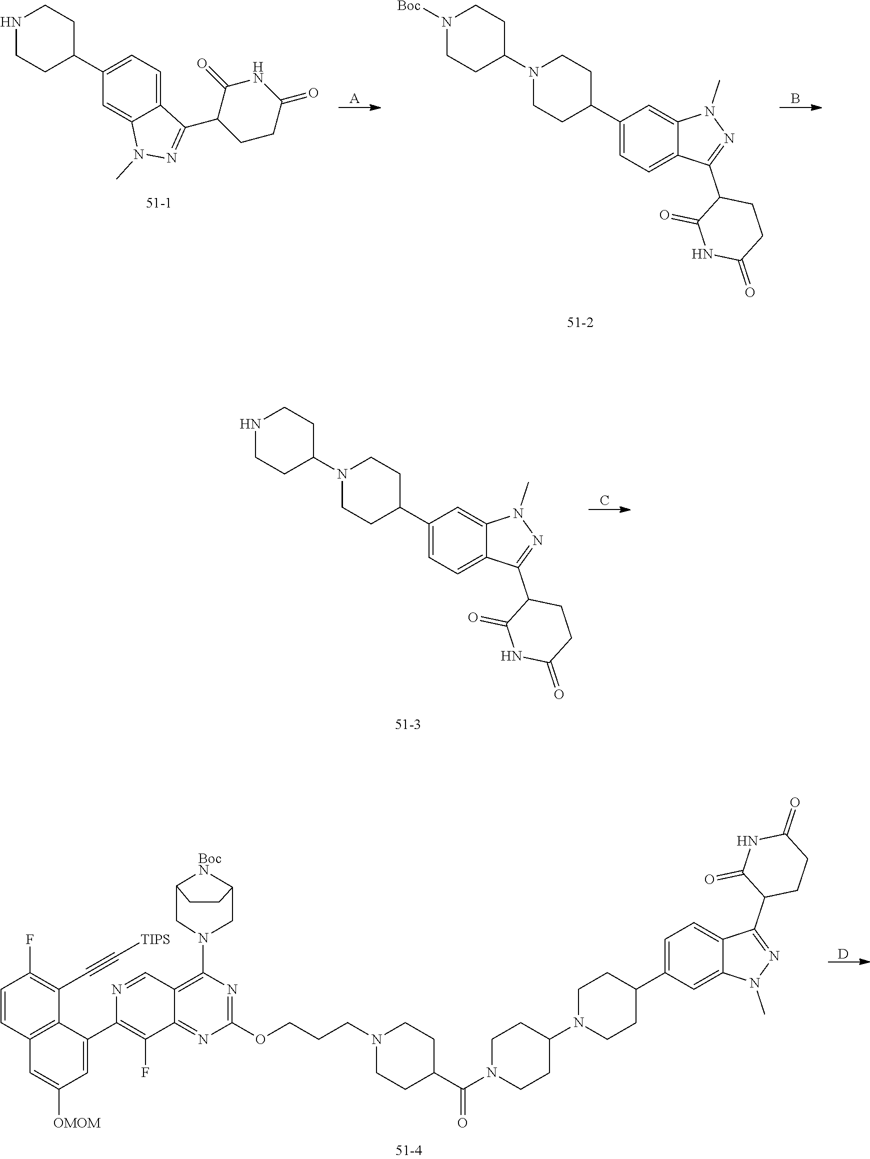

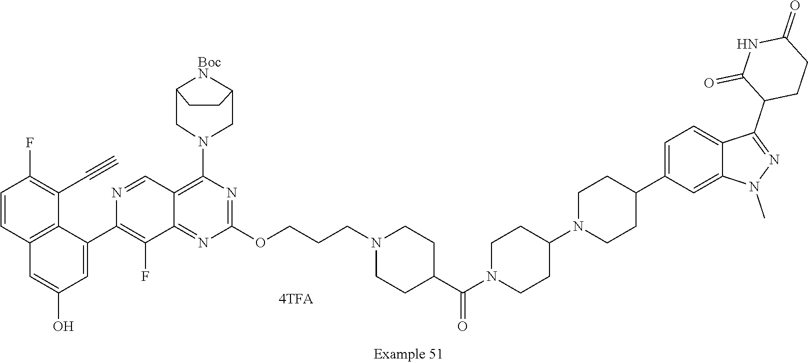 [SMILES: C#Cc1c(F)ccc2cc(O)cc(-c3ncc4c(N5CC6CCC(C5)N6C(=O)OC(C)(C)C)nc(OCCCN5CCC(C(=O)N6CCC(N7CCC(c8ccc9c(C%10CCC(=O)NC%10=O)nn(C)c9c8)CC7)CC6)CC5)nc4c3F)c12]

Step A: 3-(1-methyl-6-(piperidin-4-yl)-1H-indazol-3-yl)piperidine-2,6-dione (219.76 mg, 1.10 mmol, 1.2 eq) was added to a solution of 51-1 (300 mg, 919.14 μmol, 1 eq) in 1.5 mL of THF and 6 mL of MeOH, followed by addition of 2 drops of AcOH. The mixture was stirred at 55° C. for 1 h, then treated with sodium cyanoborohydride (173.28 mg, 2.76 mmol, 3 eq). The mixture was stirred at this temperature overnight, then concentrated. The residue was purified by column chromatography (MeOH/DCM=1/9) to afford 51-2 (155 mg, 33.1% yield). m/z (ESI): 510.37 [M+H]⁺.

Step B: HCl in dioxane (4 M, 1.03 mL, 14 eq) was added to a solution of 51-2 (150 mg, 294.33 μmol, 1 eq) in 2 mL of DCM and 2 mL of MeOH. The mixture was stirred at room temperature for 3 h, then concentrated to afford white solid 51-3 (130 mg, 99% yield). m/z (ESI): 410.70 [M+H]⁺.

Step C: Chloro-N,N,N′,N′-tetramethylformamidiniumhexafluorophosphate (54.35 mg, 194.09 μmol, 1.5 eq) was added to a solution of intermediate b (100 mg, 129.39 μmol, 1 eq), followed by addition of 51-3 (69.25 mg, 155.27 μmol, 1.2 eq), DIEA (0.5 mL) and 1-methylimidazole (53.05 mg, 646.97 μmol, 5 eq). The mixture was stirred at room temperature overnight, then concentrated. The residue was purified by column chromatography (MeOH/DCM=1/9˜1/4) to afford 51-4 (25 mg, 14.1% yield). m/z (ESI): 1164.81 [M+H]⁺.

Step D: HCl in dioxane (4 M, 0.5 mL) was added to a solution of 51-4 (25 mg, 17.18 μmol, 1 eq) in 2 mL of DCM. The mixture was stirred at room temperature for 30 min, then concentrated. The residue was purified by Prep-HPLC to afford light yellow solid (13.5 mg, 49.4% yield). ¹H NMR (500 MHz, Methanol-d4) δ 1.68-1.71 (m, 1H), 1.83-1.85 (m, 1H), 2.01-2.12 (m, 4H), 2.14-2.18 (m, 6H), 2.24-2.27 (m, 4H), 2.32-2.36 (m, 4H), 2.47-2.51 (m, 1H), 2.72-2.81 (m, 3H), 3.11-3.13 (m, 4H), 3.27-3.29 (m, 3H), 3.40-3.42 (m, 3H), 3.55-3.64 (m, 1H), 3.72-3.77 (m, 4H), 3.96-4.01 (m, 1H), 4.05 (s, 3H), 4.31-4.32 (m, 3H), 4.40 (dd, J=9.2, 5.1 Hz, 1H), 4.63-4.67 (m, 2H), 4.78 (d, J=12.6 Hz, 1H), 4.86 (d, J=13.9 Hz, 2H), 7.13 (d, J=8.4 Hz, 1H), 7.25 (s, 1H), 7.36-7.41 (m, 3H), 7.73 (d, J=8.4 Hz, 1H), 7.91 (dd, J=8.5, 5.8 Hz, 1H), 9.11 (s, 1H). m/z (ESI): 1020.7 [M+H]⁺.

### Example 52. Synthesis of Compound 76 Salt

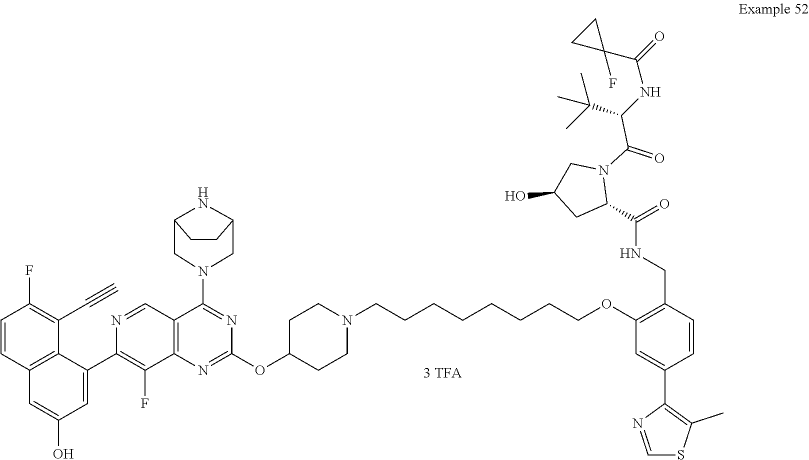 [SMILES: C#Cc1c(F)ccc2cc(O)cc(-c3ncc4c(N5CC6CCC(C5)N6)nc(OC5CCN(CCCCCCCCOc6cc(-c7ncsc7C)ccc6CNC(=O)[C@@H]6C[C@@H](O)CN6C(=O)[C@@H](NC(=O)C6(F)CC6)C(C)(C)C)CC5)nc4c3F)c12]

Target compound was synthesized according to the procedure of Example 1. ¹H NMR (500 MHz, Methanol-d4) δ 1.04 (s, 9H), 1.37-1.28 (m, 5H), 1.45-1.42 (m, 6H), 1.57 (brs, 2H), 1.70-1.65 (m, 1H), 1.88-1.78 (m, 4H), 2.04-1.99 (m, 1H), 2.24-2.11 (m, 8H), 2.38-2.35 (m, 1H), 2.49 (s, 3H), 3.24-3.12 (m, 4H), 3.39-3.34 (m, 2H), 3.55-3.53 (m, 1H), 3.71-3.64 (m, 1H), 3.80-3.78 (m, 1H), 3.87-3.85 (m, 1H), 3.99-3.91 (m, 2H), 4.08 (t, J=5 Hz, 2H), 4.28 (brs, 2H), 4.50-4.37 (m, 3H), 4.65-4.61 (m, 1H), 4.83-4.74 (m, 5H), 5.59-5.34 (m, 1H), 7.01-6.99 (m, 2H), 7.22 (s, 1H), 7.38-7.33 (m, 2H), 7.49-7.47 (m, 1H), 7.54-7.53 (m, 1H), 7.90-7.87 (m, 1H), 8.92 (s, 1H), 9.08-9.07 (m, 1H). m/z (ESI): 1213.6 [M+H]⁺.

### Example 53. Synthesis of Compound 77 Salt

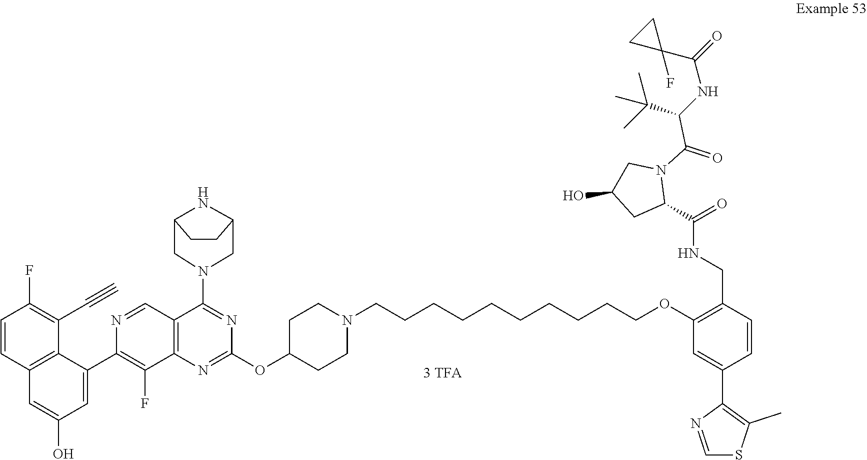 [SMILES: C#Cc1c(F)ccc2cc(O)cc(-c3ncc4c(N5CC6CCC(C5)N6)nc(OC5CCN(CCCCCCCCCCOc6cc(-c7ncsc7C)ccc6CNC(=O)[C@@H]6C[C@@H](O)CN6C(=O)[C@@H](NC(=O)C6(F)CC6)C(C)(C)C)CC5)nc4c3F)c12]

Target compound was synthesized according to the procedure of Example 1. ¹H NMR (500 MHz, Methanol-d4) δ1.04 (s, 9H), 1.40-1.27 (m, 16H), 1.55-1.53 (m, 2H), 1.86-1.77 (m, 4H), 2.04-2.00 (m, 1H), 2.27-2.10 (m, 8H), 2.39-2.36 (m, 1H), 2.49 (s, 3H), 3.25-3.13 (m, 3H), 3.39-3.34 (m, 2H), 3.56-3.53 (m, 1H), 3.71-3.64 (m, 1H), 3.87-3.78 (m, 2H), 3.99-3.91 (m, 2H), 4.07 (t, J=5 Hz, 2 h), 4.28 (brs, 2H), 4.51-4.37 (m, 3H), 4.65-4.62 (m, 1H), 4.86-4.74 (m, 4H), 5.60-5.41 (m, 1H), 7.01-6.98 (m, 2H), 7.22-7.21 (m, 1H), 7.38-7.33 (m, 2H), 7.49-7.47 (m, 1H), 7.55-7.52 (m, 1H), 7.90-7.87 (m, 1H), 8.93 (s, 1H), 9.08-9.07 (m, 1H). m/z (ESI): 1213.6 [M+H]⁺.

### Example 54. Synthesis of Compound 78 Salt

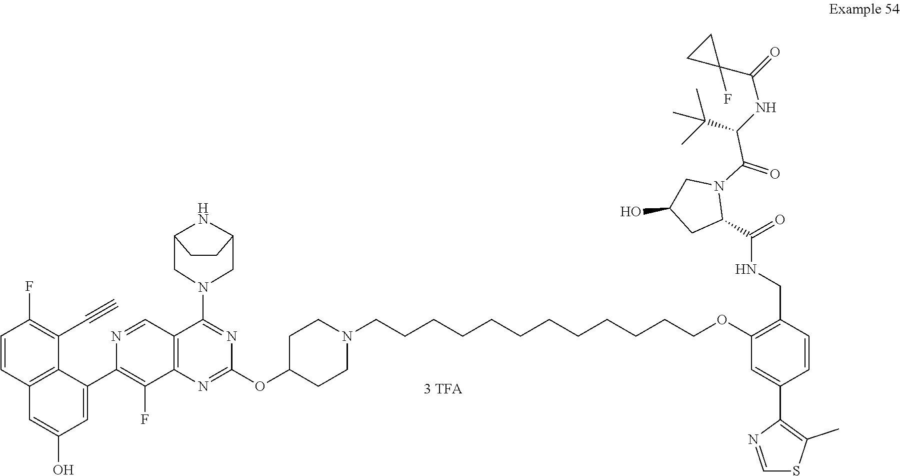 [SMILES: C#Cc1c(F)ccc2cc(O)cc(-c3ncc4c(N5CC6CCC(C5)N6)nc(OC5CCN(CCCCCCCCCCCCOc6cc(-c7ncsc7C)ccc6CNC(=O)[C@@H]6C[C@@H](O)CN6C(=O)[C@@H](NC(=O)C6(F)CC6)C(C)(C)C)CC5)nc4c3F)c12]

Target compound was synthesized according to the procedure of Example 1. ¹H NMR (500 MHz, Methanol-d4) δ1.03 (s, 9H), 1.38-1.28 (m, 18H), 1.54-1.50 (m, 2H), 1.86-1.77 (m, 4H), 2.04-1.93 (m, 1H), 2.27-2.10 (m, 7H), 2.38-2.35 (m, 1H), 2.50 (s, 3H), 3.19-3.15 (m, 2H), 3.24-3.23 (m, 4H), 3.39-3.34 (m, 2H), 3.56-3.53 (m, 1H), 3.71-3.64 (m, 1H), 3.87-3.78 (m, 2H), 4.00-3.92 (m, 2H), 4.07-4.05 (m, 2H), 4.28 (brs, 2H), 4.51-4.37 (m, 3H), 4.65-4.62 (m, 1H), 4.85-4.74 (m, 4H), 5.59-5.39 (m, 1H), 7.01-6.98 (m, 2H), 7.21 (brs, 1H), 7.38-7.32 (m, 2H), 7.49-7.47 (m, 1H), 7.55-7.53 (m, 1H), 7.90-7.87 (m, 1H), 8.92-8.91 (m, 1H), 9.08-9.07 (m, 1H). m/z (ESI): 1241.7 [M+H]⁺.

### Example 55. Synthesis of Compound 80 Salt

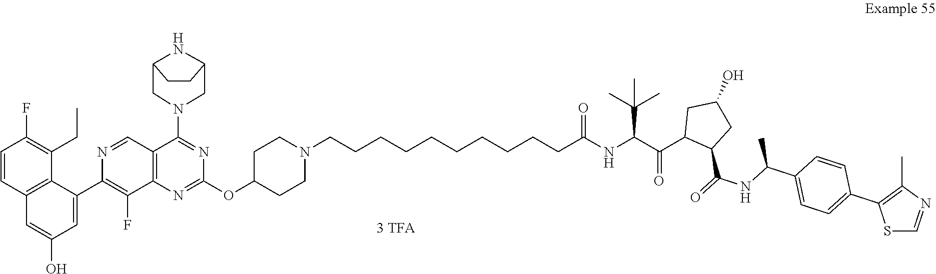 [SMILES: CCc1c(F)ccc2cc(O)cc(-c3ncc4c(N5CC6CCC(C5)N6)nc(OC5CCN(CCCCCCCCCCC(=O)N[C@H](C(=O)C6C[C@H](O)C[C@H]6C(=O)N[C@@H](C)c6ccc(-c7scnc7C)cc6)C(C)(C)C)CC5)nc4c3F)c12]

Target compound was synthesized according to the procedure of Example 1. ¹H NMR (500 MHz, Methanol-d4) δ 0.81-0.78 (m, 3H), 1.04 (s, 9H), 1.40-1.34 (m, 12H), 1.51-1.45 (m, 3H), 1.64-1.56 (m, 2H), 1.77 (brs, 2H), 2.04-1.92 (m, 2H), 2.32-2.14 (m, 10H), 2.42-2.37 (m, 1H), 2.56-2.48 (m, 4H), 3.26-3.16 (m, 3H), 3.38-3.36 (m, 1H), 3.56-3.54 (m, 1H), 3.76-3.70 (m, 2H), 4.01-3.86 (m, 3H), 4.29-4.26 (m, 2H), 4.43 (brs, 1H), 4.58-4.54 (m, 1H), 4.64-4.62 (m, 1H), 4.83-4.78 (m, 2H), 5.02-4.99 (m, 1H), 5.60-5.42 (m, 1H), 7.04 (s, 1H), 7.29-7.25 (m, 1H), 7.32-7.29 (m, 1H), 7.45-7.41 (m, 3H), 7.71-7.68 (m, 1H), 7.83-7.81 (m, 1H), 8.92-8.90 (m, 1H), 9.12-9.11 (m, 1H). m/z (ESI): 1157.8 [M+H]⁺.

### Example 56. Synthesis of Compound 82 Salt

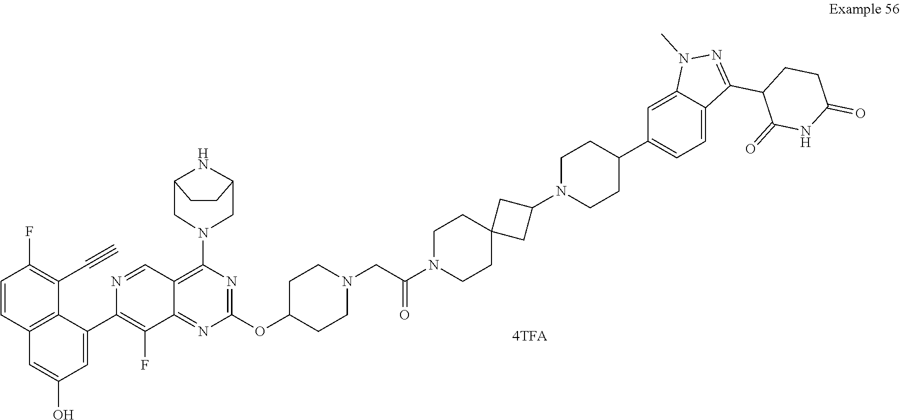 [SMILES: C#Cc1c(F)ccc2cc(O)cc(-c3ncc4c(N5CC6CCC(C5)N6)nc(OC5CCN(CC(=O)N6CCC7(CC6)CC(N6CCC(c8ccc9c(C%10CCC(=O)NC%10=O)nn(C)c9c8)CC6)C7)CC5)nc4c3F)c12]

Target compound was synthesized according to the procedure of Example 51. ¹H NMR (500 MHz, Methanol-d4) δ 1.19-1.46 (m, 3H), 1.72 (t, J=28.2 Hz, 4H), 1.98-2.27 (m, 12H), 2.29-2.61 (m, 7H), 2.61-3.20 (m, 6H), 3.21-3.53 (m, 29H), 3.52-3.93 (m, 6H), 4.02 (s, 3H), 4.23-4.45 (m, 4H), 7.10 (d, J=8.4 Hz, 1H), 7.21 (s, 1H), 7.30-7.50 (m, 3H), 7.56-8.02 (m, 2H), 9.08 (s, 1H). MS: 1333.87 [M+H]⁺.

### Example 57. Synthesis of Compound 83 Salt

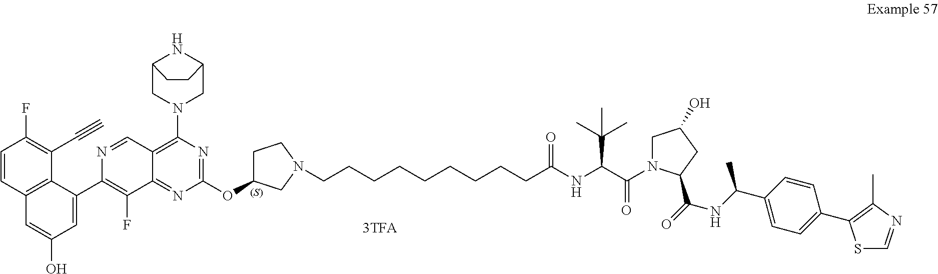

Target compound was synthesized according to the procedure of Example 4 with intermediate h as starting material. ¹H NMR (500 MHz, Methanol-d4) δ 1.03 (d, J=2.6 Hz, 9H), 1.34 (s, 8H), 1.39 (s, 3H), 1.44 (dd, J=15.0, 7.5 Hz, 2H), 1.50 (d, J=7.0 Hz, 3H), 1.58 (dd, J=14.0, 7.3 Hz, 2H), 1.68 (dd, J=10.1, 6.1 Hz, 2H), 1.75 (s, 2H), 1.88-2.00 (m, 1H), 2.11 (d, J=10.7 Hz, 2H), 2.16 (d, J=11.0 Hz, 2H), 2.28 (tt, J=15.2, 7.3 Hz, 2H), 2.48 (s, 3H), 3.11-3.15 (m, 2H), 3.26 (d, J=8.9 Hz, 2H), 3.38 (d, J=11.8 Hz, 1H), 3.54 (d, J=14.1 Hz, 1H), 3.75 (dd, J=10.9, 3.9 Hz, 1H), 3.87 (d, J=11.2 Hz, 2H), 3.98 (d, J=11.2 Hz, 2H), 4.28 (s, 2H), 4.43 (s, 1H), 4.56 (t, J=8.2 Hz, 1H), 4.58-4.68 (m, 1H), 4.81 (d, J=14.6 Hz, 2H), 5.00 (t, J=6.8 Hz, 1H), 7.21 (t, J=2.4 Hz, 1H), 7.33 (d, J=8.9 Hz, 1H), 7.40-7.46 (m, 5H), 7.88 (dd, J=9.1, 5.7 Hz, 1H), 8.91 (d, J=9.6 Hz, 1H), 9.09 (s, 1H). m/z (ESI): 1225.6 [M+H]⁺.

### Example 58. Synthesis of Compound 84 Salt

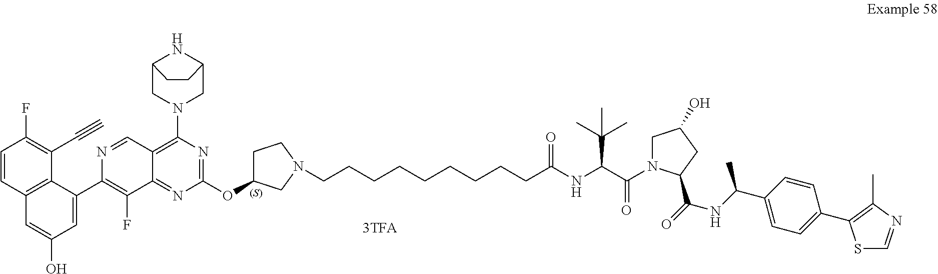

Target compound was synthesized according to the procedure of Example 4 with intermediate h as starting material. ¹H NMR (500 MHz, Methanol-d4) δ 1.04 (s, 10H), 1.32 (s, 16H), 1.50 (d, J=7.0 Hz, 4H), 1.55-1.65 (m, 3H), 1.76 (s, 2H), 1.95 (ddd, J=13.2, 8.9, 4.5 Hz, 1H), 2.11 (d, J=10.8 Hz, 2H), 2.20-2.34 (m, 3H), 2.48 (s, 3H), 3.25 (s, 1H), 3.38 (d, J=11.6 Hz, 2H), 3.54 (d, J=14.1 Hz, 1H), 3.75 (dd, J=11.3, 3.9 Hz, 1H), 3.87 (d, J=11.3 Hz, 2H), 3.93-4.04 (m, 3H), 4.28 (d, J=8.4 Hz, 2H), 4.43 (s, 1H), 4.56 (t, J=8.4 Hz, 1H), 4.63 (d, J=6.3 Hz, 1H), 4.81 (d, J=12.4 Hz, 2H), 4.97-5.07 (m, 2H), 7.21 (s, 1H), 7.33 (d, J=9.0 Hz, 1H), 7.35-7.39 (m, 2H), 7.43 (q, J=8.2 Hz, 5H), 7.88 (dd, J=9.2, 5.7 Hz, 1H), 8.92 (d, J=8.0 Hz, 1H), 9.09 (s, 1H). m/z (ESI): 1153.7 [M+H]⁺.

### Example 59. Synthesis of Compound 85 Salt

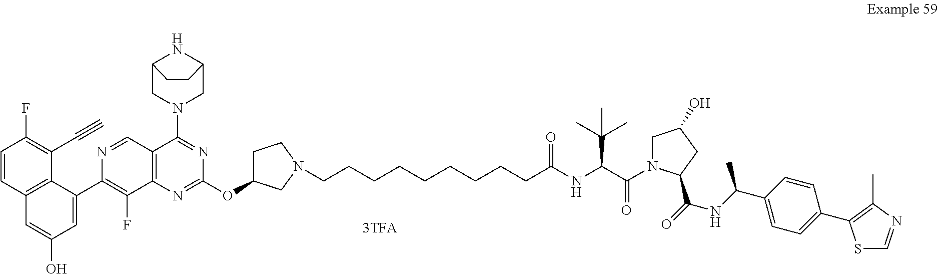 [SMILES: C#Cc1c(F)ccc2cc(O)cc(-c3ncc4c(N5CC6CCC(C5)N6)nc(O[C@H]5CCN(CCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12]

Target compound was synthesized according to the procedure of Example 51. ¹H NMR (500 MHz, Methanol-d4) δ 1.03 (s, 9H), 1.31-1.34 (m, 7H), 1.37-1.46 (m, 6H), 1.47-1.54 (m, 3H), 1.55-1.62 (m, 2H), 1.66-1.71 (m, 2H), 1.75 (s, 2H), 2.08-2.33 (m, 7H), 2.48 (s, 3H), 3.09-3.16 (m, 2H), 3.35-3.41 (m, 2H), 3.49-3.59 (m, 1H), 3.71-3.78 (m, 1H), 3.84-4.03 (m, 4H), 4.24-4.34 (m, 2H), 4.41-4.46 (m, 1H), 4.52-4.59 (m, 1H), 4.61-4.68 (m, 1H), 4.96-5.05 (m, 1H), 7.20-7.23 (m, 1H), 7.32-7.39 (m, 2H), 7.40-7.46 (m, 4H), 7.81 (d, J=9.0 Hz, 1H), 8.56 (d, J=7.4 Hz, 1H), 9.09 (s, 1H). m/z (ESI⁺): 1139.6 [M+H]⁺.

### Example 60. Synthesis of Compound 86 Salt

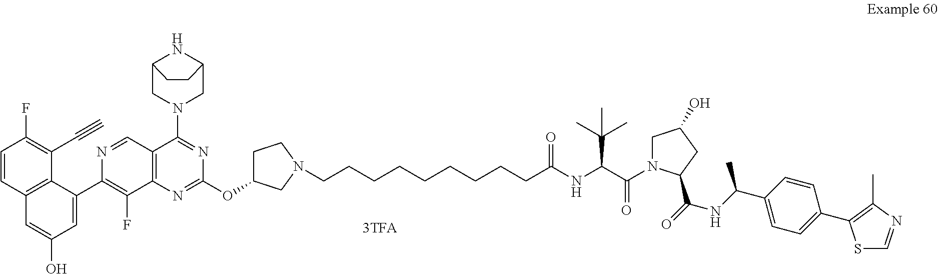 [SMILES: C#Cc1c(F)ccc2cc(O)cc(-c3ncc4c(N5CC6CCC(C5)N6)nc(O[C@@H]5CCN(CCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12]

Target compound was synthesized according to the procedure of Example 4. ¹H NMR (500 MHz, Methanol-d4) δ 1.07 (s, 9H), 1.33-1.39 (m, 7H), 1.41-1.50 (m, 6H), 1.54 (d, J=7.0 Hz, 3H), 1.58-1.66 (m, 2H), 1.68-1.75 (m, 2H), 1.76-1.86 (m, 2H), 2.11-2.35 (m, 7H), 2.51 (s, 3H), 3.10-3.22 (m, 2H), 3.25-3.31 (m, 2H), 3.38-3.43 (m, 1H), 3.54-3.63 (m, 1H), 3.74-3.81 (m, 1H), 3.88-4.07 (m, 4H), 4.27-4.33 (m, 2H), 4.47 (s, 1H), 4.57-4.62 (m, 1H), 4.64-4.69 (m, 1H), 4.78-4.84 (m, 3H), 5.01-5.09 (m, 1H), 7.25 (s, 1H), 7.35-7.43 (m, 2H), 7.44-7.53 (m, 4H), 7.92 (dd, J=9.1, 5.7 Hz, 1H), 8.95 (s, 1H), 9.13 (s, 1H). m/z (ESI⁺): 1139.6 [M+H]⁺.

### Example 61. Synthesis of Compound 87 Salt

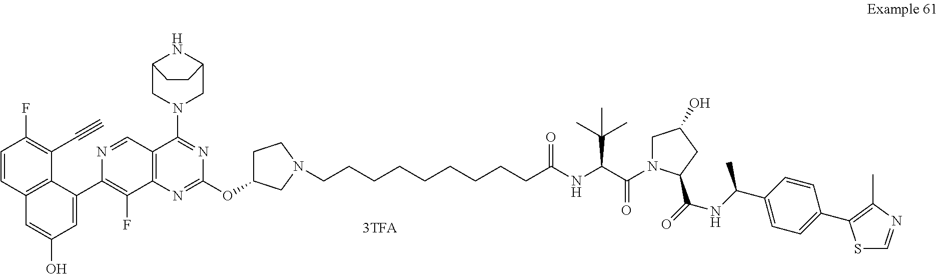 [SMILES: C#Cc1c(F)ccc2cc(O)cc(-c3ncc4c(N5CC6CCC(C5)N6)nc(O[C@@H]5CCN(CCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12]

Target compound was synthesized according to the procedure of Example 4 with intermediate h as starting material. ¹H NMR (500 MHz, Methanol-d6) δ 1.04 (s, 2H), 1.07 (s, 9H), 1.35-1.42 (m, 18H), 1.54 (d, J=6.9 Hz, 3H), 1.57-1.65 (m, 2H), 1.79 (s, 2H), 1.97-2.01 (m, 1H), 2.13-2.15 (d, J=9.9 Hz, 2H), 2.21-2.32 (m, 5H), 2.52 (s, 3H), 3.29 (s, 1H), 3.40 (d, J=11.8 Hz, 1H), 3.56-3.65 (m, 1H), 3.77-3.79 (m, 1H), 3.91 (d, J=11.1 Hz, 2H), 3.99-4.05 (m, 2H), 4.32 (s, 2H), 4.47 (s, 1H), 4.60 (t, J=8.3 Hz, 1H), 4.66 (s, 1H), 5.03-5.04 (m, 1H), 7.25 (s, 1H), 7.37 (t, J=9.0 Hz, 1H), 7.41 (s, 1H), 7.44-7.50 (m, 4H), 7.88-7.94 (m, 1H), 8.96 (s, 1H), 9.13 (s, 1H); m/z (ESI): 1153.5 [M+H]⁺.

### Example 62. Synthesis of Compound 88 Salt

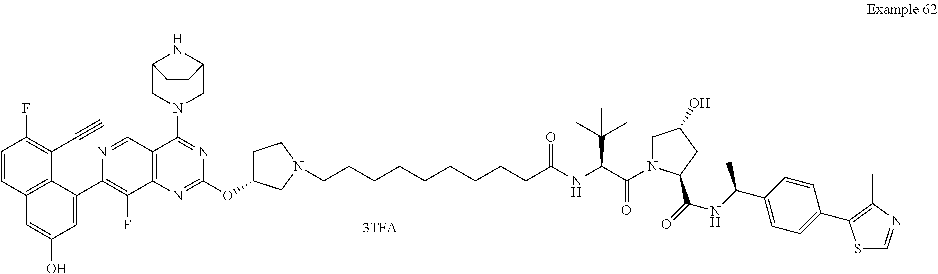 [SMILES: C#Cc1c(F)ccc2cc(O)cc(-c3ncc4c(N5CC6CCC(C5)N6)nc(O[C@@H]5CCN(CCCCCCCCCC(=O)N[C@H](C(=O)N6C[C@H](O)C[C@H]6C(=O)N[C@@H](C)c6ccc(-c7scnc7C)cc6)C(C)(C)C)C5)nc4c3F)c12]

Target compound was synthesized according to the procedure of Example 4 with intermediate h as starting material. ¹H NMR (500 MHz, Methanol-d6) δ 1.06 (s, 9H), 1.37-1.46 (m, 12H), 1.53 (d, J=6.9 Hz, 3H), 1.68-1.78 (m, 6H), 2.13-2.29 (m, 6H), 2.50 (s, 3H), 3.14-3.18 (m, 6H), 3.28 (s, 1H), 3.39 (d, J=12.2 Hz, 1H), 3.51-3.67 (m, 1H), 3.90 (d, J=10.9 Hz, 1H), 3.98-4.04 (m, 2H), 4.31 (s, 2H), 4.46 (s, 1H), 4.59 (t, J=8.2 Hz, 1H), 4.65 (d, J=5.8 Hz, 1H), 5.01-5.04 (m, 1H), 7.24 (s, 1H), 7.36 (t, J=8.9 Hz, 1H), 7.40 (s, 1H), 7.43-7.47 (m, 4H), 7.88-7.93 (m, 1H), 8.94 (s, 1H), 9.12 (s, 1H); m/z (ESI): 1125.5 [M+H]⁺.

### Example 63. Synthesis of Compound 90 Salt

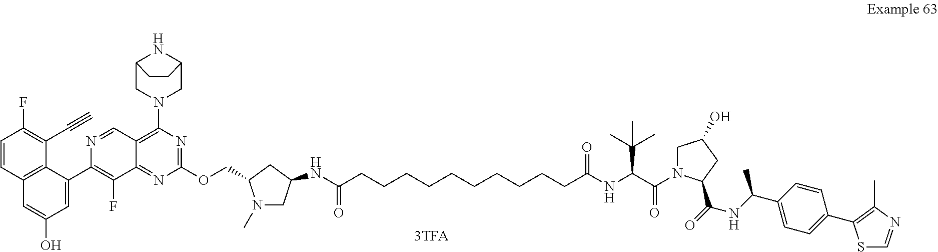 [SMILES: C#Cc1c(F)ccc2cc(O)cc(-c3ncc4c(N5CC6CCC(C5)N6)nc(OC[C@@H]5C[C@@H](NC(=O)CCCCCCCCCCC(=O)N[C@H](C(=O)N6C[C@H](O)C[C@H]6C(=O)N[C@@H](C)c6ccc(-c7scnc7C)cc6)C(C)(C)C)CN5C)nc4c3F)c12]

Target compound was synthesized according to the procedure of Example 4. ¹H NMR (500 MHz, Methanol-d4) δ 1.00-1.06 (m, 9H), 1.26-1.38 (m, 12H), 1.50 (d, J=5.0 Hz, 3H), 1.55-1.65 (m, 4H), 1.92-2.00 (m, 1H), 2.07-2.40 (m, 10H), 2.42-2.55 (m, 4H), 3.08-3.24 (m, 4H), 3.34-3.38 (m, 1H), 3.72-3.78 (m, 1H), 3.84-3.90 (m, 1H), 3.93-4.08 (m, 3H), 4.10-4.20 (m, 1H), 4.25-4.32 (m, 2H), 4.40-4.51 (m, 2H), 4.56 (d, J=10.0 Hz, 1H), 4.60-4.65 (m, 1H), 4.74-4.83 (m, 3H), 4.92-5.03 (m, 2H), 7.20-7.24 (m, 1H), 7.32-7.39 (m, 2H), 7.39-7.46 (m, 4H), 7.85-7.91 (m, 1H), 8.92 (s, 1H), 9.08-9.15 (m, 1H). m/z (ESI): 1210.6 [M+H]⁺.

### Example 64. Synthesis of Compound 91 Salt

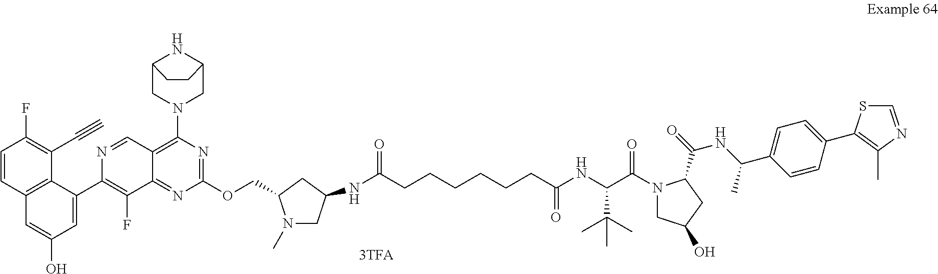 [SMILES: C#Cc1c(F)ccc2cc(O)cc(-c3ncc4c(N5CC6CCC(C5)N6)nc(OC[C@@H]5C[C@@H](NC(=O)CCCCCCC(=O)N[C@H](C(=O)N6C[C@H](O)C[C@H]6C(=O)N[C@@H](C)c6ccc(-c7scnc7C)cc6)C(C)(C)C)CN5C)nc4c3F)c12]

Target compound was synthesized according to the procedure of Example 4. ¹H NMR (400 MHz, Methanol-d4) δ 1.04 (s, 9H), 1.31-1.38 (m, 4H), 1.45-1.55 (m, 3H), 1.55-1.68 (m, 5H), 1.76-1.98 (m, 6H), 2.12-2.21 (m, 4H), 2.23-2.32 (m, 3H), 2.44-2.50 (m, 4H), 2.51 (s, 3H), 2.93-3.02 (m, 1H), 3.34-3.43 (m, 2H), 3.64-3.77 (m, 5H), 3.84-3.92 (m, 1H), 4.31-4.40 (m, 1H), 4.40-4.53 (m, 3H), 4.54-4.66 (m, 9H), 4.96-5.07 (m, 1H), 7.17-7.24 (m, 1H), 7.27-7.36 (m, 2H), 7.40-7.46 (m, 4H), 7.85 (dd, J=9.2, 5.7 Hz, 1H), 8.87 (s, 1H), 9.01 (s, 1H). m/z (ESI): 1154.7 [M+H]⁺.

### Example 65. Synthesis of Compound 92 Salt

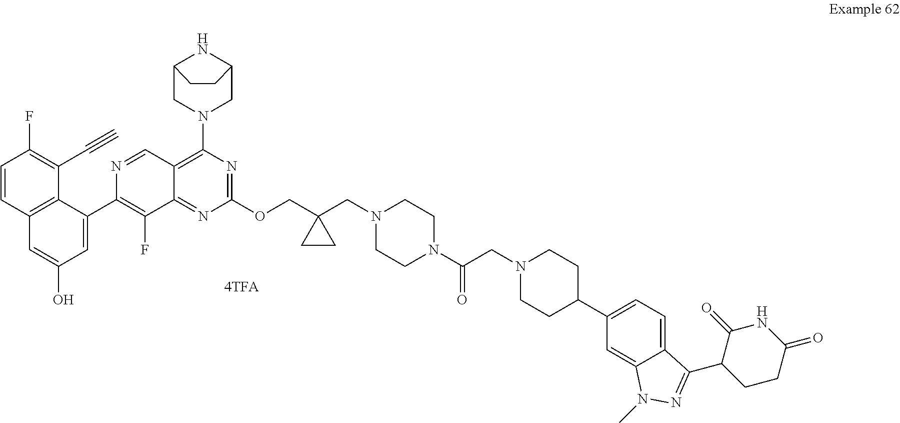 [SMILES: C#Cc1c(F)ccc2cc(O)cc(-c3ncc4c(N5CC6CCC(C5)N6)nc(OCC5(CN6CCN(C(=O)CN7CCC(c8ccc9c(C%10CCC(=O)NC%10=O)nn(C)c9c8)CC7)CC6)CC5)nc4c3F)c12]

Target compound was synthesized according to the procedure of Example 51. ¹H NMR (500 MHz, Methanol-d4) δ 0.88-0.95 (m, 2H), 0.99-1.09 (m, 2H), 1.27-1.34 (m, 1H), 2.09-2.23 (m, 8H), 2.29-2.37 (m, 1H), 2.42-2.52 (m, 1H), 2.66 (s, 4H), 2.72-2.83 (m, 2H), 3.03-3.13 (m, 2H), 3.23 (dt, J=13.0, 5.3 Hz, 2H), 3.32-3.53 (m, 4H), 3.73-3.87 (m, 3H), 3.89-4.01 (m, 2H), 4.02 (s, 3H), 4.29 (d, J=13.1 Hz, 2H), 4.34-4.40 (m, 2H), 4.44 (d, J=11.7 Hz, 1H), 4.61 (d, J=11.7 Hz, 1H), 4.80 (s, 2H), 7.11 (d, J=8.5 Hz, 1H), 7.22 (d, J=2.6 Hz, 1H), 7.36 (dd, J=20.2, 11.5 Hz, 3H), 7.71 (d, J=8.4 Hz, 1H), 7.86 (d, J=7.2 Hz, 1H), 9.09 (s, 1H). m/z (ESI): 978.5 [M+H]⁺.

### Example 66. Synthesis of Compound 93 Salt

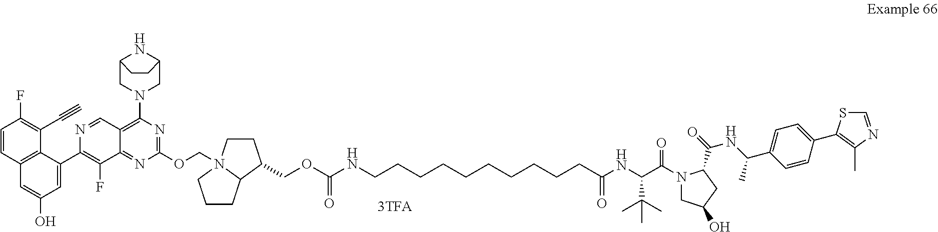

Target compound was synthesized according to the procedure of Example 4. ¹H NMR (500 MHz, Methanol-d4) δ 1.02 (s, 9H), 1.29 (brs, 14H), 1.42-1.48 (m, 2H), 1.50 (d, J=7.0 Hz, 3H), 1.54-1.61 (m, 5H), 1.92-2.00 (m, 1H), 2.05-2.31 (m, 2H), 2.33-2.45 (m, 2H), 2.48 (s, 3H), 3.06 (t, J=7.4 Hz, 2H), 3.34 (s, 1H), 3.39-3.47 (m, 1H), 3.55-3.64 (m, 1H), 3.72-3.79 (m, 1H), 3.85-3.90 (m, 1H), 3.91-4.05 (m, 2H), 4.18-4.25 (m, 1H), 4.26-4.31 (m, 2H), 4.33-4.40 (m, 1H), 4.41-4.49 (m, 2H), 4.56 (t, J=8.4 Hz, 1H), 4.60-4.69 (m, 2H), 4.71-4.77 (m, 1H), 4.97-5.04 (m, 1H), 7.22 (s, 1H), 7.32-7.39 (m, 2H), 7.43 (q, J=8.1 Hz, 4H), 7.78 (d, J=8.7 Hz, 0.5H), 7.89 (dd, J=9.3, 5.7 Hz, 1H), 8.54 (d, J=7.5 Hz, 0.5H), 8.93 (s, 1H), 9.12 (s, 1H). m/z (ESI): 1266.7 [M+H]⁺.

### Example 67. Synthesis of Compound 94 Salt

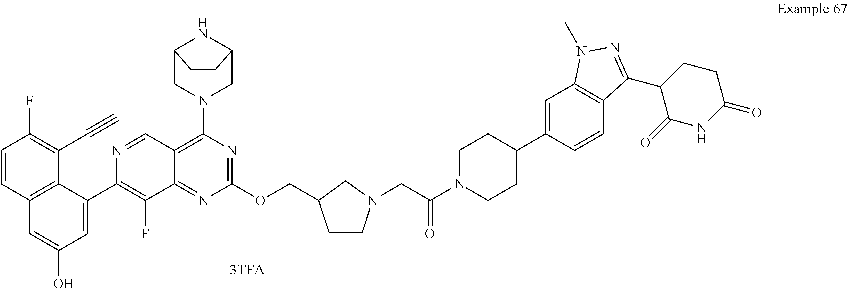 [SMILES: C#Cc1c(F)ccc2cc(O)cc(-c3ncc4c(N5CC6CCC(C5)N6)nc(OCC5CCN(CC(=O)N6CCC(c7ccc8c(C9CCC(=O)NC9=O)nn(C)c8c7)CC6)C5)nc4c3F)c12]

Target compound was synthesized according to the procedure of Example 51. ¹H NMR (500 MHz, Methanol-d4) δ 1.35 (d, J=16.2 Hz, 5H), 1.64 (t, J=7.1 Hz, 1H), 1.76 (d, J=15.4 Hz, 1H), 1.86 (s, 1H), 1.96-2.11 (m, 3H), 2.21 (d, J=19.9 Hz, 5H), 2.36 (dt, J=13.5, 6.3 Hz, 2H), 2.50 (dq, J=13.3, 5.9, 3.7 Hz, 2H), 2.79 (dt, J=14.7, 5.8 Hz, 2H), 2.90 (t, J=12.7 Hz, 1H), 3.06 (s, 2H), 3.40 (s, 1H), 3.48 (d, J=1.6 Hz, 1H), 3.86 (d, J=13.7 Hz, 2H), 4.02 (d, J=8.1 Hz, 4H), 4.32 (d, J=10.1 Hz, 2H), 4.36-4.43 (m, 1H), 4.53 (dd, J=33.1, 11.9 Hz, 2H), 4.62-4.76 (m, 3H), 7.03-7.15 (m, 1H), 7.25 (d, J=3.0 Hz, 1H), 7.33-7.44 (m, 3H), 7.69 (d, J=9.1 Hz, 1H), 7.91 (s, 1H), 9.12 (s, 1H). m/z (ESI): 909.5 [M+H]⁺.

### Example 68. Synthesis of Compound 95 Salt

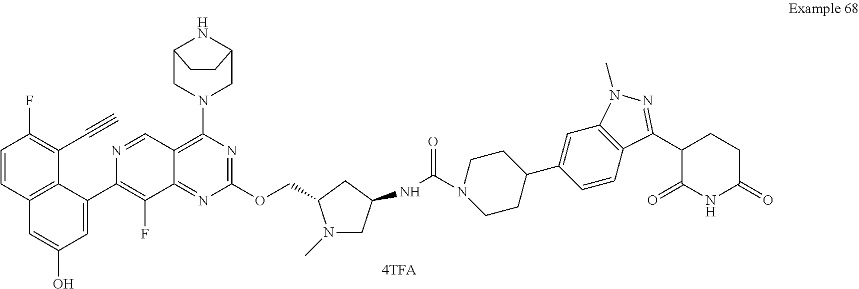 [SMILES: C#Cc1c(F)ccc2cc(O)cc(-c3ncc4c(N5CC6CCC(C5)N6)nc(OC[C@@H]5C[C@@H](NC(=O)N6CCC(c7ccc8c(C9CCC(=O)NC9=O)nn(C)c8c7)CC6)CN5C)nc4c3F)c12]

Target compound was synthesized according to the procedure of Example 51. ¹H NMR (500 MHz, Methanol-d4) δ 1.32 (d, J=4.4 Hz, 4H), 1.73 (tt, J=12.7, 7.0 Hz, 2H), 1.96 (d, J=12.8 Hz, 2H), 2.06 (d, J=6.2 Hz, 1H), 2.16-2.21 (m, 3H), 2.34 (dd, J=13.6, 6.2 Hz, 1H), 2.48 (ddd, J=22.6, 11.1, 5.6 Hz, 3H), 2.74-2.82 (m, 2H), 2.99 (t, J=13.2 Hz, 3H), 3.19 (s, 3H), 3.40 (d, J=16.9 Hz, 1H), 4.02 (s, 3H), 4.22 (d, J=13.0 Hz, 3H), 4.32 (d, J=9.4 Hz, 2H), 4.38 (dd, J=9.2, 5.2 Hz, 1H), 4.46 (s, 1H), 5.00 (d, J=12.4 Hz, 2H), 7.10 (d, J=8.5 Hz, 1H), 7.25 (d, J=2.6 Hz, 1H), 7.34-7.44 (m, 3H), 7.68 (d, J=8.5 Hz, 1H), 7.91 (dd, J=9.2, 5.7 Hz, 1H), 9.15 (d, J=2.6 Hz, 1H). m/z (ESI): 924.5 [M+H]⁺.

### Example 69. Synthesis of Compound 96 Salt

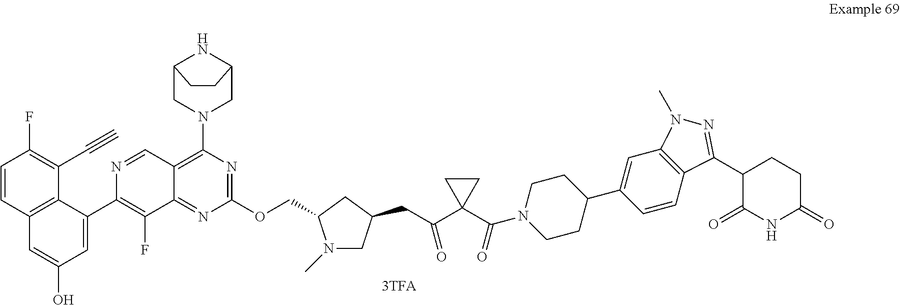 [SMILES: C#Cc1c(F)ccc2cc(O)cc(-c3ncc4c(N5CC6CCC(C5)N6)nc(OC[C@@H]5C[C@@H](CC(=O)C6(C(=O)N7CCC(c8ccc9c(C%10CCC(=O)NC%10=O)nn(C)c9c8)CC7)CC6)CN5C)nc4c3F)c12]

Target compound was synthesized according to the procedure of Example 4. ¹H NMR (500 MHz, Methanol-d4) δ 1.25-1.39 (m, 7H), 1.43 (s, 2H), 1.66-1.80 (m, 2H), 1.94 (s, 2H), 2.12 (d, J=9.7 Hz, 4H), 2.30 (dd, J=13.8, 6.4 Hz, 1H), 2.37-2.50 (m, 3H), 2.69-2.78 (m, 2H), 2.98 (d, J=10.3 Hz, 2H), 3.15 (d, J=12.1 Hz, 3H), 3.37 (d, J=13.9 Hz, 2H), 3.48 (d, J=30.9 Hz, 1H), 3.94 (d, J=15.0 Hz, 2H), 3.97-4.00 (m, 4H), 4.16 (d, J=31.8 Hz, 2H), 4.26 (s, 2H), 4.34 (dd, J=8.2, 4.5 Hz, 1H), 4.51 (s, 1H), 4.94 (d, J=10.7 Hz, 2H), 7.06 (d, J=8.6 Hz, 1H), 7.21 (d, J=2.6 Hz, 1H), 7.33 (t, J=9.4 Hz, 3H), 7.63 (d, J=8.4 Hz, 1H), 7.85 (dd, J=9.0, 5.9 Hz, 1H), 9.10 (d, J=4.1 Hz, 1H). m/z (ESI): 992.6 [M+H]⁺.

### Example 70. Synthesis of Compound 97 Salt

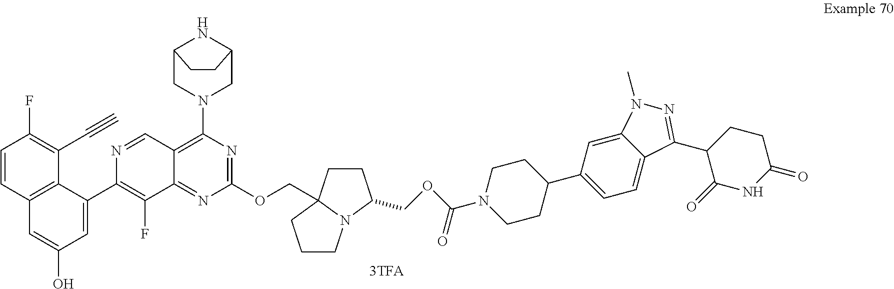 [SMILES: C#Cc1c(F)ccc2cc(O)cc(-c3ncc4c(N5CC6CCC(C5)N6)nc(OCC56CCCN5[C@@H](COC(=O)N5CCC(c7ccc8c(C9CCC(=O)NC9=O)nn(C)c8c7)CC5)CC6)nc4c3F)c12]

Target compound was synthesized according to the procedure of Example 4. ¹H NMR (500 MHz, Methanol-d4) δ 1.38-1.46 (m, 1H), 1.62-1.85 (m, 9H), 1.99-2.07 (m, 1H), 2.17-2.26 (m, 1H), 2.27-2.35 (m, 1H), 2.40-2.50 (m, 1H), 2.66-3.07 (m, 8H), 3.20-3.27 (m, 1H), 3.33-3.37 (m, 2H), 3.51-3.75 (m, 5H), 3.98 (s, 3H), 4.24-4.43 (m, 7H), 4.54-4.66 (m, 2H), 7.06 (d, J=8.7 Hz, 1H), 7.20 (s, 1H), 7.24-7.40 (m, 3H), 7.62 (d, J=8.6 Hz, 1H), 7.76-7.89 (m, 1H), 8.99 (s, 1H). m/z (ESI): 965.4 [M+H]⁺.

### Example 71. Synthesis of Compound 98 Salt

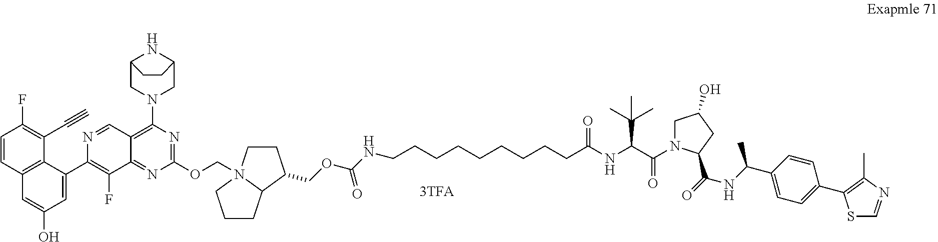

Target compound was synthesized according to the procedure of Example 4. ¹H NMR (500 MHz, Methanol-d4) δ 1.03 (s, 9H), 1.31-1.29 (m, 14H), 1.42-1.47 (m, 2H), 1.48-1.53 (m, 3H), 1.54-1.62 (m, 2H), 1.92-2.00 (m, 1H), 2.06-2.26 (m, 14H), 2.32-2.44 (m, 2H), 2.48 (s, 3H), 3.06 (t, J=7.4 Hz, 2H), 3.39-3.47 (m, 1H), 3.54-3.63 (m, 1H), 3.72-3.78 (m, 1H), 3.84-3.90 (m, 1H), 3.92-4.07 (m, 2H), 4.18-4.39 (m, 5H), 4.40-4.51 (m, 2H), 4.56 (t, J=8.2 Hz, 1H), 4.60-4.69 (m, 2H), 4.70-4.76 (m, 1H), 4.95-5.05 (m, 1H), 7.22 (s, 1H), 7.31-7.39 (m, 2H), 7.43 (q, J=8.0 Hz, 4H), 7.77 (d, J=8.9 Hz, 0.5H), 7.88 (t, J=7.5 Hz, 1H), 8.52 (d, J=7.2 Hz, 0.5H), 8.92 (s, 1H), 9.12 (s, 1H). m/z (ESI): 1252.6 [M+H]⁺.

### Example 72. Synthesis of Compound 99 Salt

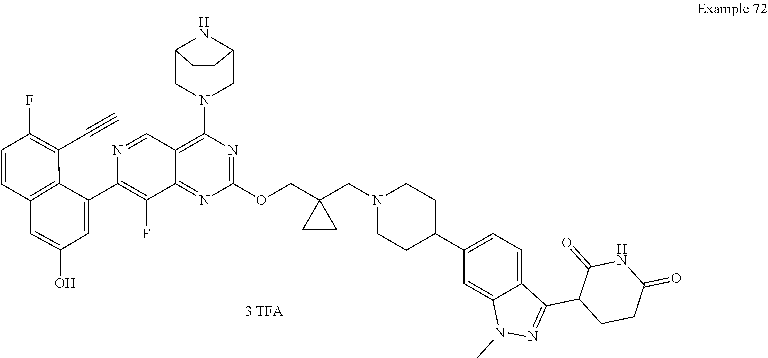 [SMILES: C#Cc1c(F)ccc2cc(O)cc(-c3ncc4c(N5CC6CCC(C5)N6)nc(OCC5(CN6CCC(c7ccc8c(C9CCC(=O)NC9=O)nn(C)c8c7)CC6)CC5)nc4c3F)c12]

Target compound was synthesized according to the procedure of Example 80. ¹H NMR (500 MHz, Methanol-d4) δ 2.11 (s, 4H), 2.15-2.28 (m, 4H), 2.30-2.38 (m, 1H), 2.41-2.51 (m, 1H), 2.72-2.86 (m, 2H), 3.01-3.10 (m, 1H), 3.13-3.25 (m, 3H), 3.41 (s, 1H), 3.49 (t, J=13.1 Hz, 1H), 3.80 (d, J=6.8 Hz, 3H), 3.86-3.99 (m, 3H), 4.06-4.14 (m, 1H), 4.19 (s, 2H), 4.33-4.46 (m, 2H), 4.70-4.84 (m, 3H), 6.98 (d, J=8.6 Hz, 1H), 7.11-7.19 (m, 1H), 7.19-7.24 (m, 2H), 7.34 (d, J=10.8, 2.6 Hz, 1H), 7.56 (d, J=8.4 Hz, 1H), 7.70-7.82 (m, 1H), 9.10 (s, 1H). m/z (ESI): 852.5 [M+H]⁺.

### Example 73. Synthesis of Compound 100 Salt

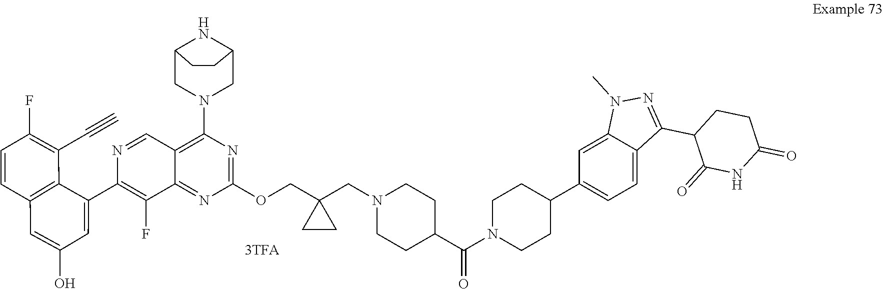 [SMILES: C#Cc1c(F)ccc2cc(O)cc(-c3ncc4c(N5CC6CCC(C5)N6)nc(OCC5(CN6CCC(C(=O)N7CCC(c8ccc9c(C%10CCC(=O)NC%10=O)nn(C)c9c8)CC7)CC6)CC5)nc4c3F)c12]

Target compound was synthesized according to the procedure of Example 80. ¹H NMR (500 MHz, Methanol-d4) δ 1.29 (d, J=4.4 Hz, 3H), 1.63-1.82 (m, 2H), 1.99 (t, J=12.4 Hz, 2H), 2.14 (d, J=30.3 Hz, 8H), 2.26-2.36 (m, 1H), 2.46 (d, J=8.8 Hz, 1H), 2.77 (tt, J=14.4, 8.5 Hz, 4H), 2.96-3.06 (m, 1H), 3.06-3.19 (m, 3H), 3.26 (s, 1H), 3.36 (d, J=14.7 Hz, 3H), 3.95 (d, J=13.2 Hz, 2H), 3.99 (s, 3H), 4.18 (s, 1H), 4.20 (s, 1H), 4.27-4.40 (m, 3H), 4.53 (dd, J=36.1, 17.6 Hz, 2H), 4.71 (d, J=13.3 Hz, 1H), 4.81 (s, 1H), 7.08 (d, J=8.5 Hz, 1H), 7.21 (d, J=2.5 Hz, 1H), 7.28-7.39 (m, 3H), 7.65 (d, J=8.4 Hz, 1H), 7.85 (dd, J=9.2, 5.7 Hz, 1H), 9.10 (s, 1H). m/z (ESI): 964.6 [M+H]⁺.

### Example 74. Synthesis of Compound 101

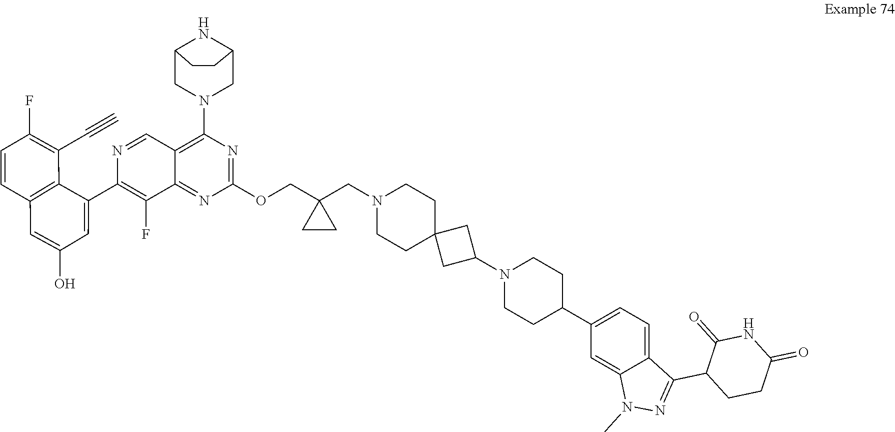 [SMILES: C#Cc1c(F)ccc2cc(O)cc(-c3ncc4c(N5CC6CCC(C5)N6)nc(OCC5(CN6CCC7(CC6)CC(N6CCC(c8ccc9c(C%10CCC(=O)NC%10=O)nn(C)c9c8)CC6)C7)CC5)nc4c3F)c12]

Target compound was synthesized according to the procedure of Example 80. ¹H NMR (500 MHz, Methanol-d4) δ 1.61 (s, 1H), 1.70-1.80 (m, 4H), 1.84-1.96 (m, 8H), 2.06-2.16 (m, 3H), 2.28-2.47 (m, 4H), 2.57-2.68 (m, 2H), 2.74-2.81 (m, 2H), 2.93-3.05 (m, 4H), 3.34 (s, 3H), 3.37-3.44 (m, 3H), 3.49-3.59 (m, 2H), 3.61-3.77 (m, 5H), 3.94-4.10 (m, 3H), 4.37 (s, 1H), 4.49-4.70 (m, 2H), 5.53 (s, 1H), 7.06 (s, 1H), 7.16-7.41 (m, 4H), 7.64 (s, 1H), 7.82 (s, 1H), 9.00 (s, 1H). m/z (ESI): 1018.7 [M+H]⁺.

### Example 75. Synthesis of Compound 102 Salt

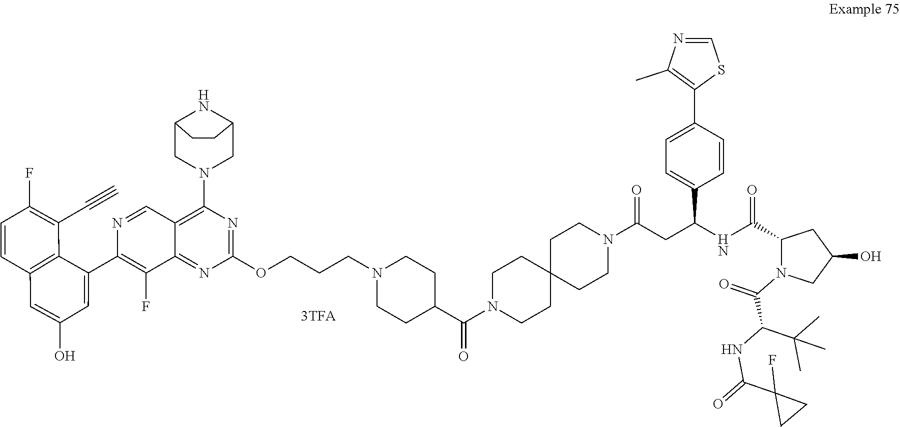 [SMILES: C#Cc1c(F)ccc2cc(O)cc(-c3ncc4c(N5CC6CCC(C5)N6)nc(OCCCN5CCC(C(=O)N6CCC7(CCN(C(=O)C[C@H](NC(=O)[C@@H]8C[C@@H](O)CN8C(=O)[C@@H](NC(=O)C8(F)CC8)C(C)(C)C)c8ccc(-c9scnc9C)cc8)CC7)CC6)CC5)nc4c3F)c12]

Target compound was synthesized according to the procedure of Example 80. ¹H NMR (500 MHz, Methanol-d4) δ 1.03-1.12 (m, 9H), 1.29-1.51 (m, 14H), 1.99 (m, 5H), 2.16 (m, 5H), 2.31 (m, 2H), 2.49 (s, 3H), 3.05 (m, 4H), 3.36 (m, 2H), 3.51 (m, 8H), 3.67-3.85 (m, 4H), 3.95 (m, 2H), 4.28 (d, J=10.1 Hz, 2H), 4.45 (s, 1H), 4.60 (m, 3H), 4.72-4.82 (m, 4H), 5.36 (m, 1H), 7.21 (d, J=2.5 Hz, 1H), 7.32-7.39 (m, 2H), 7.47 (m, 4H), 7.89 (m, 1H), 8.90 (s, 1H), 9.08 (s, 1H). m/z (ESI⁺): 1321.8 [M+H]⁺.

### Example 76. Synthesis of Compound 103 Salt

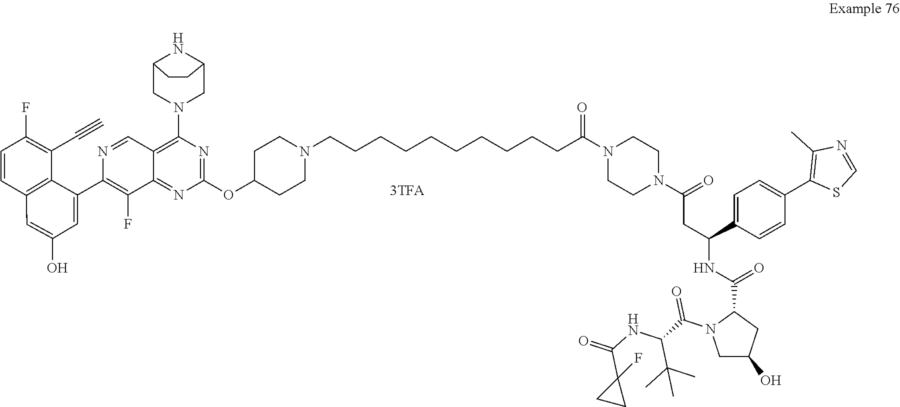 [SMILES: C#Cc1c(F)ccc2cc(O)cc(-c3ncc4c(N5CC6CCC(C5)N6)nc(OC5CCN(CCCCCCCCCCC(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)CC5)nc4c3F)c12]

Target compound was synthesized according to the procedure of Example 4. ¹H NMR (500 MHz, Methanol-d4) δ 1.00-1.06 (m, 9H), 1.26-1.38 (m, 12H), 1.50 (d, J=5.0 Hz, 3H), 1.55-1.65 (m, 4H), 1.92-2.00 (m, 1H), 2.07-2.40 (m, 10H), 2.42-2.55 (m, 4H), 3.08-3.24 (m, 4H), 3.34-3.38 (m, 1H), 3.72-3.78 (m, 1H), 3.84-3.90 (m, 1H), 3.93-4.08 (m, 3H), 4.10-4.20 (m, 1H), 4.25-4.32 (m, 2H), 4.40-4.51 (m, 2H), 4.56 (d, J=10.0 Hz, 1H), 4.60-4.65 (m, 1H), 4.74-4.83 (m, 3H), 4.92-5.03 (m, 2H), 7.20-7.24 (m, 1H), 7.32-7.39 (m, 2H), 7.39-7.46 (m, 4H), 7.85-7.91 (m, 1H), 8.92 (s, 1H), 9.08-9.15 (m, 1H). m/z (ESI): 1210.6 [M+H]⁺.

### Example 77. Synthesis of Compound 104 Salt

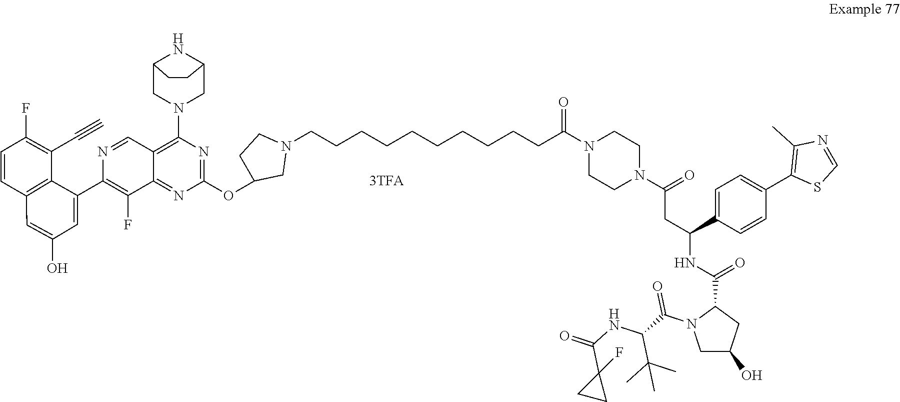 [SMILES: C#Cc1c(F)ccc2cc(O)cc(-c3ncc4c(N5CC6CCC(C5)N6)nc(OC5CCN(CCCCCCCCCCC(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)C5)nc4c3F)c12]

Target compound was synthesized according to the procedure of Example 4. ¹H NMR (500 MHz, Methanol-d4) δ 1.00-1.06 (m, 9H), 1.26-1.38 (m, 12H), 1.50 (d, J=5.0 Hz, 3H), 1.55-1.65 (m, 4H), 1.92-2.00 (m, 1H), 2.07-2.40 (m, 10H), 2.42-2.55 (m, 4H), 3.08-3.24 (m, 4H), 3.34-3.38 (m, 1H), 3.72-3.78 (m, 1H), 3.84-3.90 (m, 1H), 3.93-4.08 (m, 3H), 4.10-4.20 (m, 1H), 4.25-4.32 (m, 2H), 4.40-4.51 (m, 2H), 4.56 (d, J=10.0 Hz, 1H), 4.60-4.65 (m, 1H), 4.74-4.83 (m, 3H), 4.92-5.03 (m, 2H), 7.20-7.24 (m, 1H), 7.32-7.39 (m, 2H), 7.39-7.46 (m, 4H), 7.85-7.91 (m, 1H), 8.92 (s, 1H), 9.08-9.15 (m, 1H). m/z (ESI): 1210.6 [M+H]⁺.

### Example 78. Synthesis of Compound 105 Salt

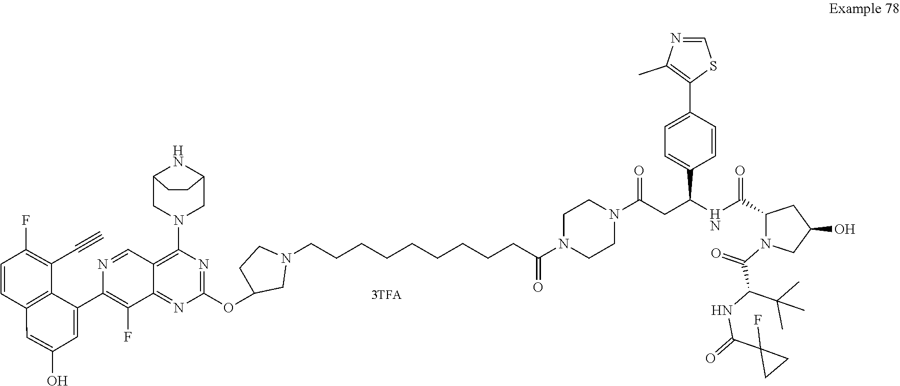 [SMILES: C#Cc1c(F)ccc2cc(O)cc(-c3ncc4c(N5CC6CCC(C5)N6)nc(OC5CCN(CCCCCCCCCC(=O)N6CCN(C(=O)C[C@H](NC(=O)[C@@H]7C[C@@H](O)CN7C(=O)[C@@H](NC(=O)C7(F)CC7)C(C)(C)C)c7ccc(-c8scnc8C)cc7)CC6)C5)nc4c3F)c12]

Target compound was synthesized according to the procedure of Example 4. ¹H NMR (500 MHz, Methanol-d4) δ 1.05 (m, 9H), 1.28-1.42 (m, 16H), 1.56 (s, 2H), 1.75 (m, 2H), 1.96 (m, 1H), 2.16 (m, 5H), 2.36 (m, 2H), 2.47 (m, 3H), 3.00-3.06 (m, 2H), 3.11-3.26 (m, 3H), 3.38 (m, 2H), 3.44-3.65 (m, 9H), 3.75-3.84 (m, 2H), 3.98 (m, 3H), 4.29 (m, 2H), 4.45 (s, 1H), 4.56 (m, 1H), 4.71-4.84 (m, 4H), 5.38 (m, 1H), 7.22 (m, 1H), 7.32-7.39 (m, 2H), 7.48 (m, 4H), 7.88 (dd, J=9.1, 5.6 Hz, 1H), 8.90 (s, 1H), 9.09 (s, 1H). m/z (ESI⁺): 1323.7 [M+H]⁺.

### Example 79. Synthesis of Compound 106

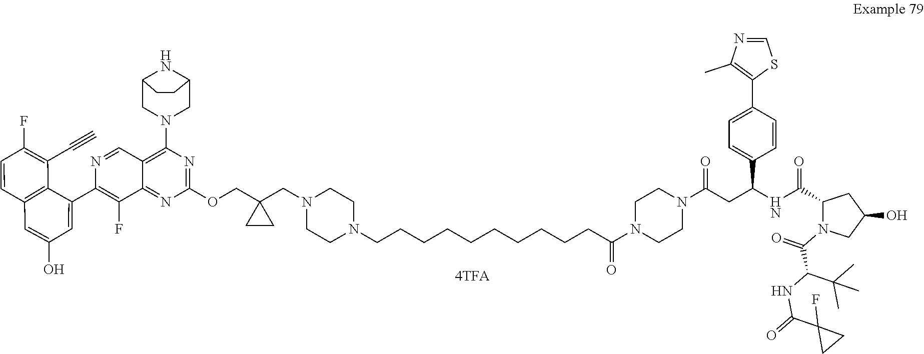 [SMILES: C#Cc1c(F)ccc2cc(O)cc(-c3ncc4c(N5CC6CCC(C5)N6)nc(OCC5(CN6CCN(CCCCCCCCCCC(=O)N7CCN(C(=O)C[C@H](NC(=O)[C@@H]8C[C@@H](O)CN8C(=O)[C@@H](NC(=O)C8(F)CC8)C(C)(C)C)c8ccc(-c9scnc9C)cc8)CC7)CC6)CC5)nc4c3F)c12]

Target compound was synthesized according to the procedure of Example 4. ¹H NMR (500 MHz, Methanol-d4) δ 0.69-0.74 (m, 2H), 0.82-0.94 (m, 2H), 1.05 (m, 9H), 1.21-1.37 (m, 18H), 1.50-1.59 (m, 2H), 1.63-1.72 (m, 2H), 1.92-2.01 (m, 1H), 2.17 (m, 5H), 2.35 (m, 2H), 2.47 (m, 3H), 2.82 (m, 1H), 2.98-3.13 (m, 5H), 3.35 (m, 2H), 3.38-3.66 (m, 13H), 3.76-3.92 (m, 2H), 4.01 (, 1H), 4.22-4.34 (m, 2H), 4.37-4.47 (m, 2H), 4.53-4.61 (m, 2H), 4.72-4.85 (m, 4H), 5.39 (m, 1H), 7.23 (s, 1H), 7.31-7.39 (m, 2H), 7.44-7.49 (m, 4H), 7.89 (dd, J=9.2, 5.7 Hz, 1H), 8.91 (s, 1H), 9.07 (s, 1H). m/z (ESI⁺): 1421.1 [M+H]⁺.

### Example 80. Synthesis of Compound 107

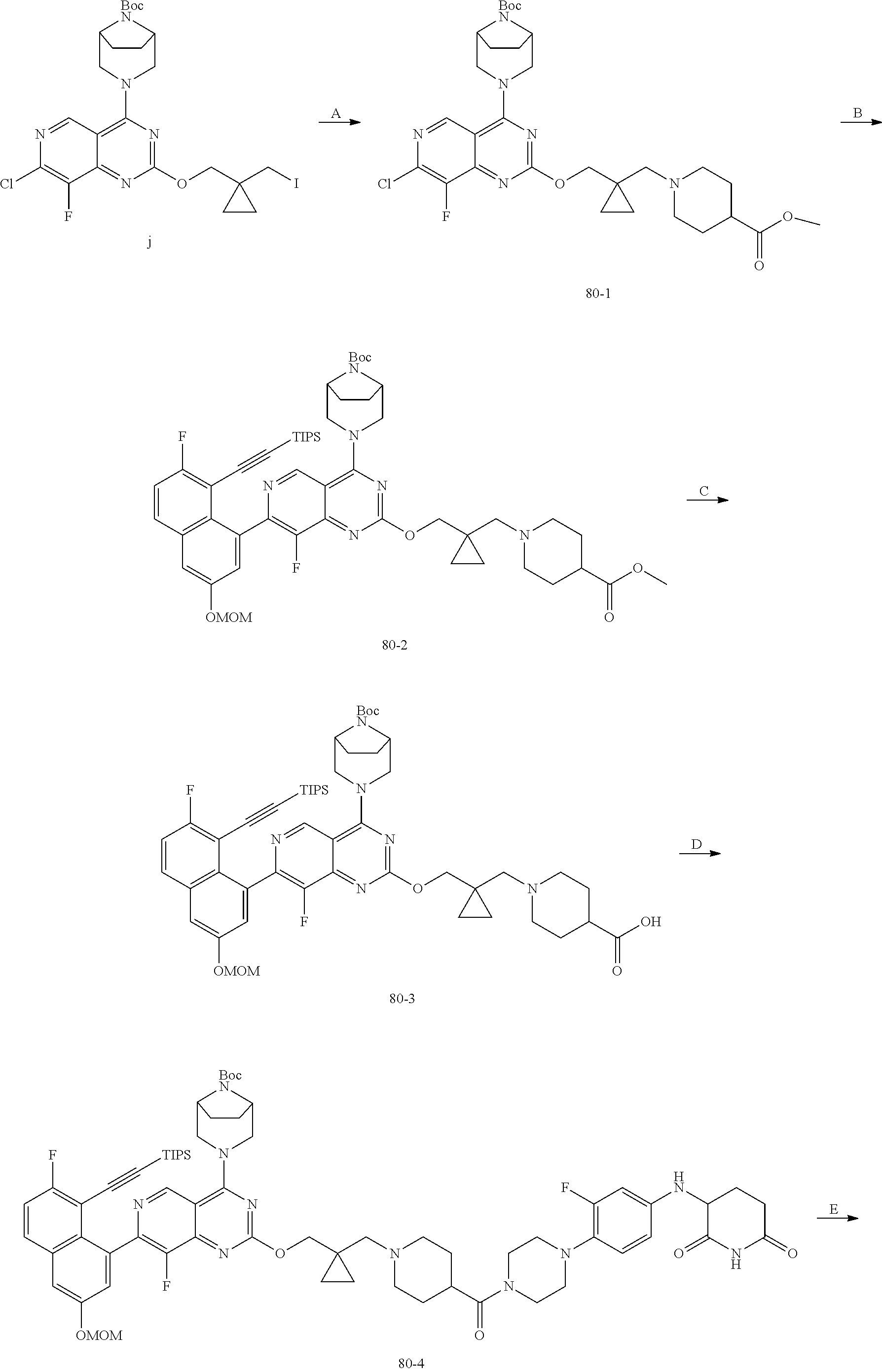

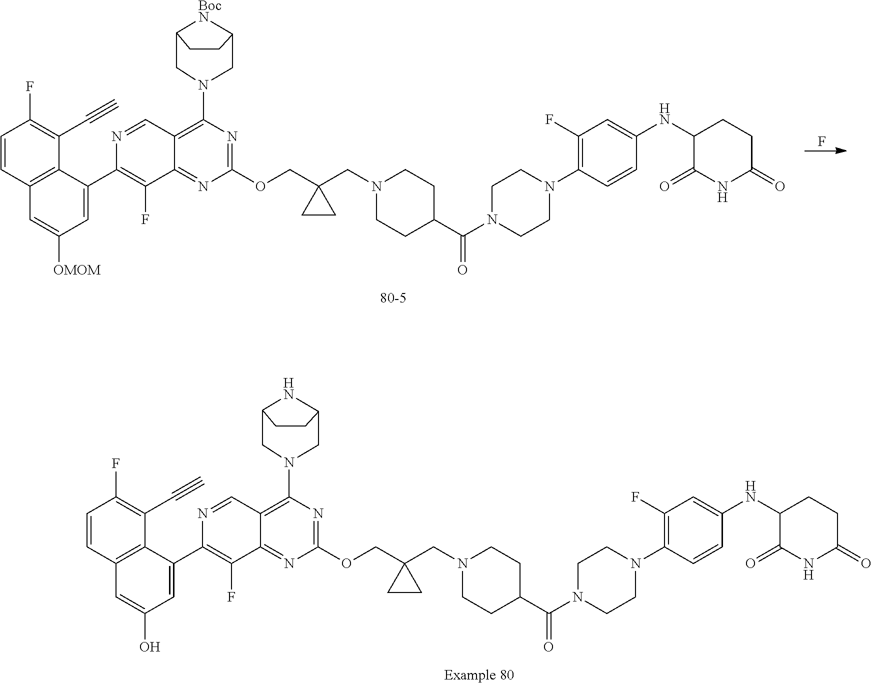

Step A: Methyl isonipecotate (200 mg, 1.40 mmol, 1 eq) was added to a solution of intermediate j (889.02 mg, 1.47 mmol, 1.05 eq) in 10 mL of DMF, followed by addition of K₂CO₃ (580.48 mg, 4.20 mmol, 3 eq). The mixture was heated to 50° C. and stirred for 2 h, then cooled to room temperature. The mixture was treated with NH₄Cl aqueous and EtOAc, and stirred for 5 min, then the organic layer was separated. The organic layer was washed with brine, dried over Na₂SO₄ and filtered. The filtrate was concentrated and the residue was purified by column chromatography (MeOH/DCM=1/19) to afford 80-1 (650 mg, 75% yield).

Step B: To a solution of 80-1 (650 mg, 1.05 mmol, 1 eq) in 20 mL of THF/Water (10/3) was added K₃PO₄ (667.72 mg, 3.15 mmol, 3 eq), cataCXium A Pd-G3 (152.86 mg, 209.97 μmol, 0.2 eq) and ((2-fluoro-6-(methoxymethoxy)-8-(4,4,5,5-tetramethyl-1,3,2-dioxaborolan-2-yl)naphthalen-1-yl)ethynyl)triisopropylsilane (645.71 mg, 1.26 mmol, 1.2 eq). The mixture was heated to 100° C. and stirred for 3 h under nitrogen atmosphere, then cooled to room temperature. The mixture was treated with EtOAc and water and stirred for 10 min, then the organic layer was separated. The organic layer was washed with brine, dried over Na₂SO₄ and filtered. The filtrate was concentrated and the residue was purified by column chromatography (MeOH/DCM=3/97) to afford yellow solid 80-2 (870 mg, 85% yield).

Step C: LiOH (49.42 mg, 2.06 mmol, 10 eq) was added to a solution of compound 80-2 (200.00 mg, 0.21 mmol, 1.0 eq) in 6 mL of MeOH and 1 mL of water. The mixture was heated to 50° C. and stirred for 1 h, then cooled to room temperature and concentrated. The pH of residue was adjusted to 4-5 and extracted with EA. The combined organic layer was washed with brine, dried over Na₂SO₄ and filtered. The filtrate was concentrated and the residue was purified by column chromatography (MeOH/DCM=1/10) to afford 80-3 (170 mg, 86% yield).

Step D: 3-((3-fluoro-4-(piperazin-1-yl)phenyl)amino)piperidine-2,6-dione (65.42 mg, 0.21 mmol, 1.2 eq) was added to a solution of 80-3 (170.00 mg, 0.18 mmol, 1.0 eq) in 4 mL of DMF, followed by addition of HATU (101.51 mg, 0.27 mmol, 1.5 eq) and DIPEA (115.00 mg, 0.89 mmol, 154.99 μL, 5 eq). The mixture was stirred at room temperature for 10 min. The mixture was treated with NH₄Cl aqueous and EtOAc, and stirred for 5 min, then separated the organic layer. The organic layer was washed with brine, dried over Na₂SO₄ and filtered. The filtrate was concentrated and the residue was purified by column chromatography (MeOH/DCM=1/10) to afford 80-4 (90 mg, 40% yield). m/z (ESI): 1244.62 [M+H]⁺.

Step E: TBAF (1 M, 361.87 μL, 361.87 μmol, 5 eq) was added to a solution of 80-4 (90 mg, 72.37 μmol, 1 eq) in 2 mL of THF. The mixture was stirred at room temperature for 2 h, then concentrated. The residue was purified by column chromatography (MeOH/DCM=1/10) to afford 80-5 (65 mg, 82.6% yield). m/z (ESI): 1087.97 [M+H]⁺.

Step F: HCl in EtOAc (4 M, 0.6 mL) was added to a solution of 80-5 (65 mg, 59.79 μmol, 1 eq) in 3 mL of DCM. The mixture was concentrated, and the residue was purified by Prep-HPLC to afford yellow solid (30 mg, 35.7% yield, purity 91.43%). ¹H NMR (500 MHz, Methanol-d6) δ 0.91-0.94 (m, 2H), 1.03 (s, 2H), 1.97-1.98 (m, 1H), 2.13-2.19 (m, 9H), 2.32 (d, J=12.7 Hz, 1H), 2.73-2.87 (m, 2H), 3.01-3.16 (m, 7H), 3.29-3.34 (m, 1H), 3.37-3.40 (m, 2H), 3.79 (s, 4H), 4.00 (t, J=14.2 Hz, 4H), 4.29-4.35 (m, 3H), 4.48-4.63 (m, 2H), 4.80-4.84 (m, 1H), 6.55 (d, J=8.3 Hz, 1H), 6.60 (d, J=14.4 Hz, 1H), 6.95-6.97 (m, 1H), 7.25 (s, 1H), 7.36 (t, J=8.9 Hz, 1H), 7.40 (s, 1H), 7.88-7.94 (m, 1H), 9.13 (s, 1H). m/z (ESI): 943.78 [M+H]⁺.

### Example 81. Synthesis of Compound 108

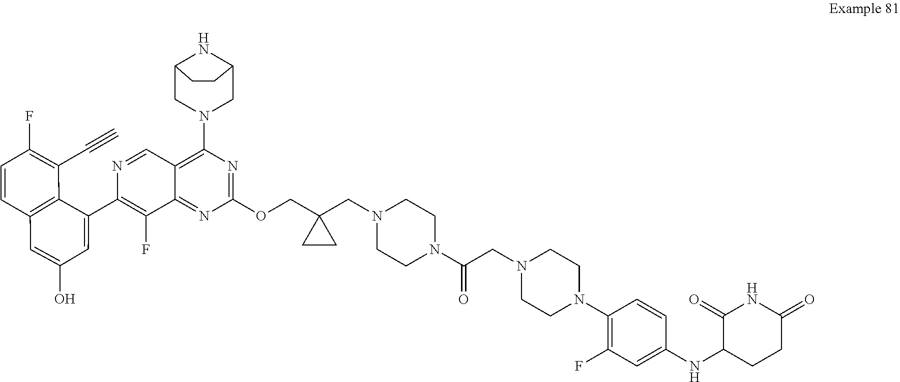 [SMILES: C#Cc1c(F)ccc2cc(O)cc(-c3ncc4c(N5CC6CCC(C5)N6)nc(OCC5(CN6CCN(C(=O)CN7CCN(c8ccc(NC9CCC(=O)NC9=O)cc8F)CC7)CC6)CC5)nc4c3F)c12]

Target compound was synthesized according to the procedure of Example 80. ¹H NMR (500 MHz, Methanol-d6) δ 0.54 (s, 2H), 0.76 (s, 2H), 1.82-1.97 (m, 5H), 2.30-2.36 (m, 1H), 2.48-2.54 (m, 4H), 2.65 (s, 5H), 2.96 (s, 4H), 3.28 (s, 2H), 3.38 (s, 1H), 3.61-3.65 (m, 2H), 3.71-3.78 (m, 4H), 4.49 (s, 2H), 4.64 (s, 8H), 6.48 (d, J=8.7 Hz, 1H), 6.55 (d, J=14.5 Hz, 1H), 6.85 (t, J=9.0 Hz, 1H), 7.24 (s, 1H), 7.33-7.38 (m, 2H), 7.89 (dd, J=8.9, 5.9 Hz, 1H), 9.03 (s, 1H). m/z (ESI): 958.6 [M+H]⁺.

### Example 82. Synthesis of Compound 109

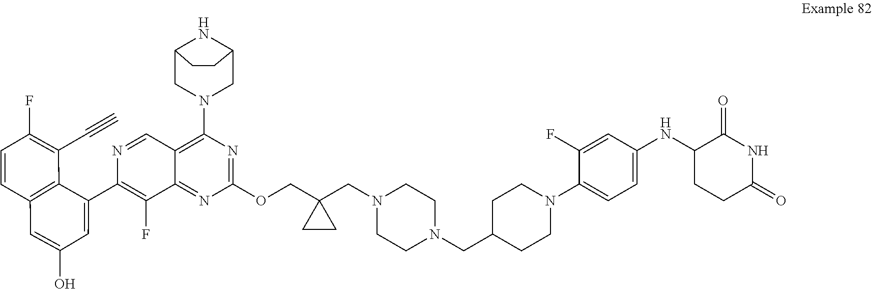 [SMILES: C#Cc1c(F)ccc2cc(O)cc(-c3ncc4c(N5CC6CCC(C5)N6)nc(OCC5(CN6CCN(CC7CCN(c8ccc(NC9CCC(=O)NC9=O)cc8F)CC7)CC6)CC5)nc4c3F)c12]

Target compound was synthesized according to the procedure of Example 80. ¹H NMR (500 MHz, Methanol-d4) δ 0.60 (s, 2H), 0.79 (s, 2H), 0.90 (t, J=6.9 Hz, 1H), 1.03 (t, J=7.4 Hz, 1H), 1.36-1.45 (m, 3H), 1.58-1.73 (m, 2H), 1.80 (d, J=13.0 Hz, 2H), 1.87-2.06 (m, 6H), 2.27-2.35 (m, 1H), 2.49 (s, 2H), 2.60 (t, J=11.7 Hz, 2H), 2.69 (t, J=4.3 Hz, 1H), 2.73 (t, J=4.2 Hz, 1H), 2.76-2.85 (m, 4H), 3.15-3.27 (m, 2H), 3.39 (s, 1H), 3.78 (d, J=13.1 Hz, 2H), 3.86 (s, 2H), 4.23 (dd, J=11.8, 4.8 Hz, 1H), 4.35-4.43 (m, 1H), 4.47-4.54 (m, 1H), 4.58-4.73 (m, 3H), 6.45-6.58 (m, 2H), 6.90 (t, J=9.2 Hz, 1H), 7.21 (s, 1H), 7.28-7.40 (m, 2H), 7.87 (dd, J=9.1, 5.8 Hz, 1H), 9.03 (s, 1H). m/z (ESI): 929.4 [M+H]⁺.

### Example 83. Synthesis of Compound 110

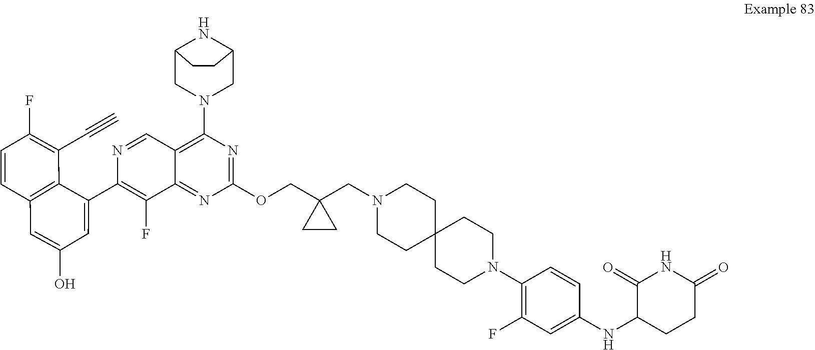 [SMILES: C#Cc1c(F)ccc2cc(O)cc(-c3ncc4c(N5CC6CCC(C5)N6)nc(OCC5(CN6CCC7(CC6)CCN(c6ccc(NC8CCC(=O)NC8=O)cc6F)CC7)CC5)nc4c3F)c12]

Target compound was synthesized according to the procedure of Example 80. ¹H NMR (500 MHz, Methanol-d4) δ 9.10 (s, 1H), 7.89 (d, J=8.1 Hz, 1H), 7.43-7.29 (m, 2H), 7.22 (s, 1H), 7.02 (d, J=9.7 Hz, 1H), 6.62-6.49 (m, 2H), 4.62 (d, J=12.1 Hz, 1H), 4.45 (d, J=12.2 Hz, 1H), 4.28 (d, J=10.6 Hz, 3H), 3.95 (dd, J=23.8, 14.3 Hz, 2H), 3.72 (dd, J=44.8, 11.6 Hz, 3H), 3.43 (d, J=16.9 Hz, 3H), 3.17 (s, 2H), 3.04 (s, 4H), 2.80 (d, J=13.8 Hz, 1H), 2.72 (d, J=18.0 Hz, 1H), 2.29 (s, 1H), 2.17 (d, J=17.7 Hz, 4H), 2.03 (s, 4H), 1.89 (s, 2H), 1.78 (s, 2H), 1.61 (s, 3H), 1.02 (d, J=13.3 Hz, 3H), 0.89 (d, J=14.5 Hz, 3H). m/z (ESI⁺): 900.4 [M+H]⁺.

### Example 84. Synthesis of Compound 111

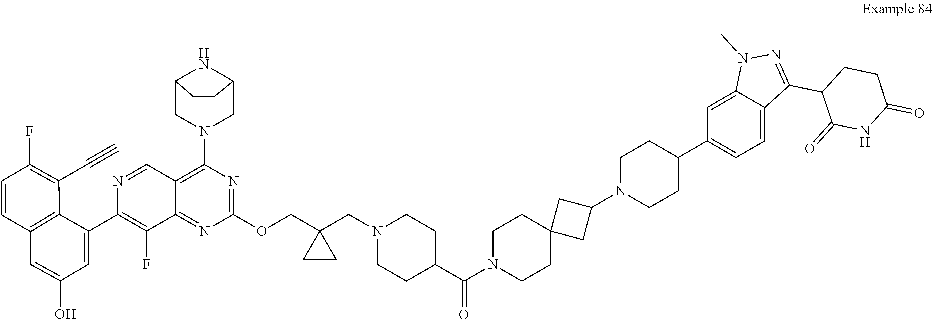 [SMILES: C#Cc1c(F)ccc2cc(O)cc(-c3ncc4c(N5CC6CCC(C5)N6)nc(OCC5(CN6CCC(C(=O)N7CCC8(CC7)CC(N7CCC(c9ccc%10c(C%11CCC(=O)NC%11=O)nn(C)c%10c9)CC7)C8)CC6)CC5)nc4c3F)c12]

Target compound was synthesized according to the procedure of Example 80. ¹H NMR (500 MHz, Methanol-d4) δ 0.51 (s, 2H), 0.72 (s, 2H), 0.90 (t, J=6.8 Hz, 1H), 1.03 (t, J=7.4 Hz, 1H), 1.30 (s, 3H), 1.42 (d, J=7.5 Hz, 1H), 1.52 (s, 1H), 1.66 (s, 4H), 1.74 (t, J=9.5 Hz, 4H), 1.79-1.91 (m, 6H), 1.91-1.98 (m, 3H), 2.03 (d, J=8.1 Hz, 4H), 2.09-2.15 (m, 2H), 2.31 (dd, J=13.5, 6.2 Hz, 1H), 2.38-2.55 (m, 3H), 2.69-2.79 (m, 3H), 3.20-3.27 (m, 1H), 3.38 (s, 2H), 3.54 (s, 2H), 3.70 (dd, J=26.6, 11.0 Hz, 4H), 4.01 (s, 3H), 4.35 (dd, J=9.2, 5.1 Hz, 1H), 4.42 (s, 2H), 4.60 (s, 3H), 7.10 (d, J=8.5 Hz, 1H), 7.23 (d, J=2.6 Hz, 1H), 7.31 (d, J=8.9 Hz, 1H), 7.35 (dd, J=5.8, 3.2 Hz, 2H), 7.65 (d, J=8.4 Hz, 1H), 7.86 (dd, J=9.1, 5.6 Hz, 1H), 9.00 (s, 1H). m/z (ESI): 1087.8 [M+H]⁺.

### Example 85. Synthesis of Compound 112

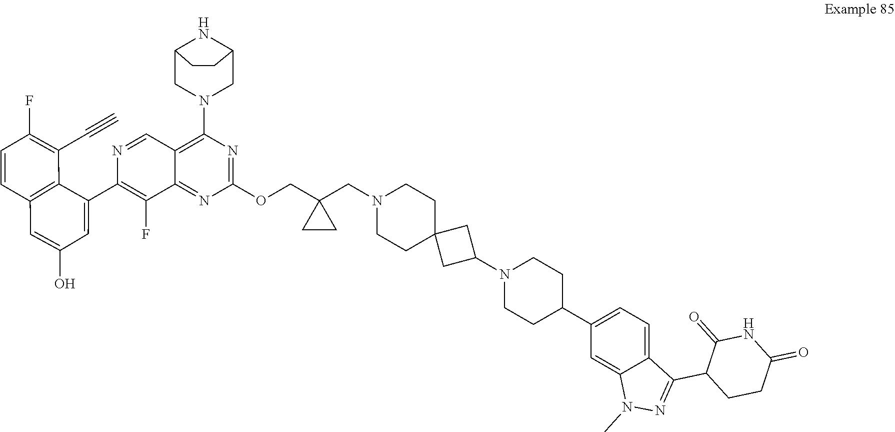 [SMILES: C#Cc1c(F)ccc2cc(O)cc(-c3ncc4c(N5CC6CCC(C5)N6)nc(OCC5(CN6CCC7(CC6)CC(N6CCC(c8ccc9c(C%10CCC(=O)NC%10=O)nn(C)c9c8)CC6)C7)CC5)nc4c3F)c12]

Target compound was synthesized according to the procedure of Example 80. ¹H NMR (500 MHz, Methanol-d4) δ 0.52 (s, 2H), 0.72 (s, 2H), 1.03 (t, J=7.4 Hz, 2H), 1.41-1.44 (m, 1H), 1.56-1.60 (m, 2H), 1.63-1.68 (m, 4H), 1.78-1.90 (m, 7H), 1.91-1.97 (m, 2H), 1.99-2.06 (m, 2H), 2.27-2.34 (m, 1H), 2.39-2.49 (m, 4H), 2.67-2.78 (m, 4H), 3.05 (d, J=11.0 Hz, 2H), 3.21-3.26 (m, 2H), 3.36 (s, 1H), 3.64-3.75 (m, 4H), 4.01 (s, 3H), 4.32-4.40 (m, 2H), 4.43-4.48 (m, 1H), 4.56 (d, J=12.7 Hz, 1H), 4.64 (d, J=12.6 Hz, 1H), 7.08 (d, J=8.5 Hz, 1H), 7.21 (d, J=2.6 Hz, 1H), 7.28-7.38 (m, 3H), 7.64 (d, J=8.5 Hz, 1H), 7.85 (dd, J=9.2, 5.7 Hz, 1H), 9.00 (s, 1H). m/z (ESI): 975.8 [M+H]⁺.

### Example 86. Synthesis of Compound 113

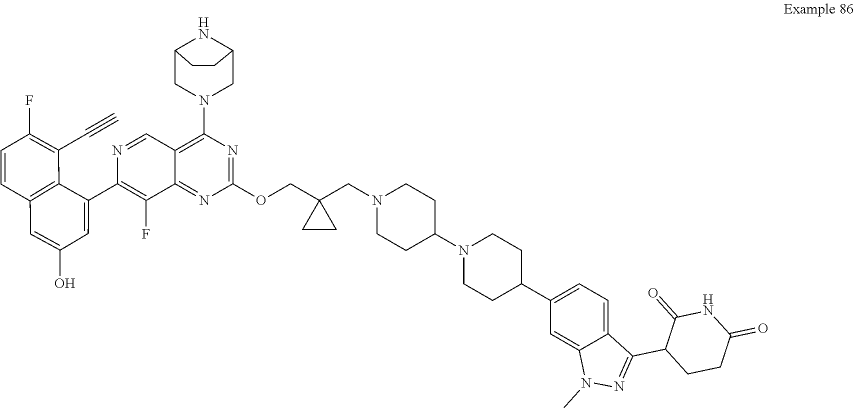 [SMILES: C#Cc1c(F)ccc2cc(O)cc(-c3ncc4c(N5CC6CCC(C5)N6)nc(OCC5(CN6CCC(N7CCC(c8ccc9c(C%10CCC(=O)NC%10=O)nn(C)c9c8)CC7)CC6)CC5)nc4c3F)c12]

Target compound was synthesized according to the procedure of Example 80. ¹H NMR (500 MHz, Methanol-d4) δ 0.52 (s, 2H), 0.73 (s, 2H), 1.03 (t, J=7.4 Hz, 2H), 1.42 (q, J=7.5 Hz, 1H), 1.57-1.70 (m, 3H), 1.77-1.92 (m, 10H), 1.97 (t, J=11.7 Hz, 2H), 2.27-2.34 (m, 1H), 2.36-2.48 (m, 5H), 2.66-2.82 (m, 3H), 3.04-3.11 (m, 2H), 3.19-3.25 (m, 2H), 3.38 (s, 1H), 3.70 (dd, J=22.9, 10.6 Hz, 4H), 4.01 (s, 3H), 4.32-4.41 (m, 2H), 4.48-4.52 (m, 1H), 4.55-4.60 (m, 1H), 4.62-4.69 (m, 1H), 7.06 (d, J=8.5 Hz, 1H), 7.21 (d, J=2.6 Hz, 1H), 7.26-7.35 (m, 3H), 7.63 (d, J=8.5 Hz, 1H), 7.83 (dd, J=9.2, 5.7 Hz, 1H), 9.00 (s, 1H). m/z (ESI): 935.7 [M+H]⁺.

### Example 87. Synthesis of Compound 114

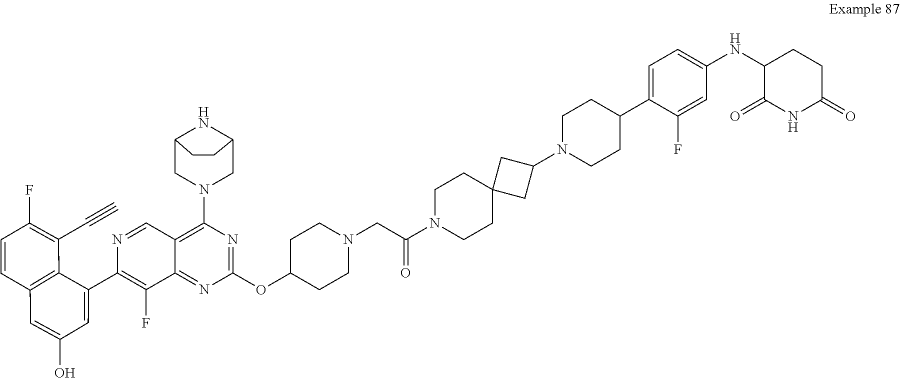 [SMILES: C#Cc1c(F)ccc2cc(O)cc(-c3ncc4c(N5CC6CCC(C5)N6)nc(OC5CCN(CC(=O)N6CCC7(CC6)CC(N6CCC(c8ccc(NC9CCC(=O)NC9=O)cc8F)CC6)C7)CC5)nc4c3F)c12]

Target compound was synthesized according to the procedure of Example 80. ¹H NMR (500 MHz, Methanol-d4) δ 1.30 (s, 5H), 1.59 (d, J=13.2 Hz, 2H), 1.65 (s, 3H), 1.73 (s, 1H), 1.84 (s, 2H), 1.88-1.90 (m, 2H), 1.95 (d, J=4.4 Hz, 2H), 2.03 (d, J=6.5 Hz, 2H), 2.14 (s, 2H), 2.22 (d, J=9.2 Hz, 2H), 2.30 (dd, J=11.4, 6.6 Hz, 2H), 2.53 (d, J=11.0 Hz, 2H), 2.68-2.74 (m, 1H), 2.85 (s, 4H), 3.25 (d, J=9.2 Hz, 2H), 3.51 (s, 2H), 3.58 (s, 2H), 3.70-3.77 (m, 4H), 4.28 (dd, J=11.9, 4.8 Hz, 1H), 4.60 (s, 6H), 5.20 (d, J=14.8 Hz, 2H), 6.43-6.55 (m, 2H), 7.03 (t, J=8.6 Hz, 1H), 7.21 (d, J=2.5 Hz, 1H), 7.29-7.39 (m, 2H), 7.86 (dd, J=9.1, 5.7 Hz, 1H), 9.01 (s, 1H). m/z (ESI): 1011.8 [M+H]⁺.

### Example 88. Synthesis of Compound 115

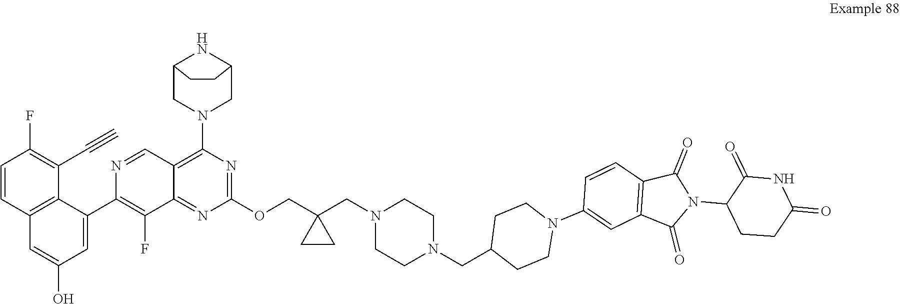 [SMILES: C#Cc1c(F)ccc2cc(O)cc(-c3ncc4c(N5CC6CCC(C5)N6)nc(OCC5(CN6CCN(CC7CCN(c8ccc9c(c8)C(=O)N(C8CCC(=O)NC8=O)C9=O)CC7)CC6)CC5)nc4c3F)c12]

Target compound was synthesized according to the procedure of Example 80. ¹H NMR (500 MHz, Methanol-d4) δ 0.51 (s, 2H), 0.72 (s, 2H), 0.90 (s, 2H), 1.30 (s, 4H), 1.56-1.65 (m, 2H), 1.87 (s, 2H), 2.00-2.04 (m, 2H), 2.20 (d, J=7.1 Hz, 3H), 2.45 (q, J=12.7 Hz, 5H), 2.74 (d, J=16.8 Hz, 3H), 2.81-2.88 (m, 2H), 2.96 (t, J=12.5 Hz, 2H), 3.37 (s, 1H), 3.68 (s, 1H), 3.69-3.76 (m, 2H), 4.00 (d, J=12.9 Hz, 2H), 4.37 (d, J=10.9 Hz, 1H), 4.49 (d, J=10.9 Hz, 1H), 4.59 (s, 2H), 4.65 (d, J=12.4 Hz, 1H), 5.03-5.09 (m, 1H), 7.16-7.23 (m, 2H), 7.28-7.37 (m, 3H), 7.65 (d, J=8.6 Hz, 1H), 7.85 (dd, J=9.1, 5.7 Hz, 1H), 9.00 (s, 1H). m/z (ESI): 966.6 [M+H]⁺.

### Example 89. Synthesis of Compound 116

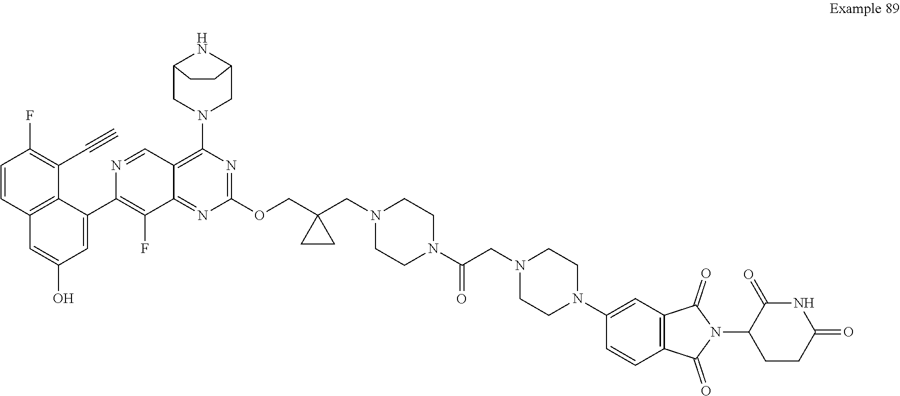 [SMILES: C#Cc1c(F)ccc2cc(O)cc(-c3ncc4c(N5CC6CCC(C5)N6)nc(OCC5(CN6CCN(C(=O)CN7CCN(c8ccc9c(c8)C(=O)N(C8CCC(=O)NC8=O)C9=O)CC7)CC6)CC5)nc4c3F)c12]

Target compound was synthesized according to the procedure of Example 80. ¹H NMR (500 MHz, DMSO-d6) δ 0.41 (s, 2H), 0.65 (s, 2H), 0.85 (t, J=6.7 Hz, 2H), 0.93 (t, J=7.3 Hz, 1H), 1.23 (s, 4H), 1.35 (s, 2H), 1.45 (s, 1H), 1.65 (s, 3H), 1.96-2.03 (m, 4H), 2.34 (d, J=17.2 Hz, 4H), 2.43 (s, 1H), 2.62 (d, J=17.9 Hz, 2H), 2.85-2.92 (m, 1H), 3.19 (s, 2H), 3.41 (s, 4H), 3.53 (s, 3H), 3.92 (s, 1H), 4.29 (s, 2H), 4.47 (d, J=12.1 Hz, 1H), 7.16 (d, J=2.5 Hz, 1H), 7.23 (d, J=9.0 Hz, 1H), 7.31-7.40 (m, 2H), 7.45 (t, J=9.0 Hz, 1H), 7.67 (d, J=8.4 Hz, 1H), 7.96 (dd, J=9.2, 6.1 Hz, 1H), 9.02 (s, 1H). m/z (ESI): 994.6 [M+H]⁺.

### Example 90. Synthesis of Compound 117

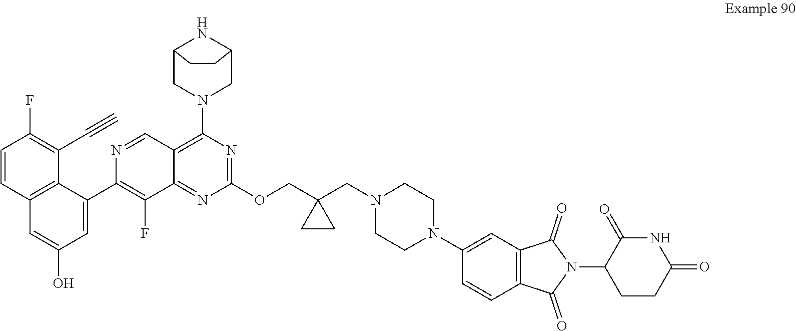 [SMILES: C#Cc1c(F)ccc2cc(O)cc(-c3ncc4c(N5CC6CCC(C5)N6)nc(OCC5(CN6CCN(c7ccc8c(c7)C(=O)N(C7CCC(=O)NC7=O)C8=O)CC6)CC5)nc4c3F)c12]

Target compound was synthesized according to the procedure of Example 80. ¹H NMR (500 MHz, DMSO-d6) δ 0.45 (s, 2H), 0.68 (d, J=4.1 Hz, 2H), 1.23 (s, 2H), 1.65 (d, J=10.4 Hz, 4H), 2.00 (dd, J=11.8, 6.5 Hz, 2H), 2.38 (d, J=3.5 Hz, 2H), 2.56 (d, J=4.7 Hz, 5H), 2.88 (ddd, J=17.8, 14.2, 5.4 Hz, 2H), 3.41 (d, J=5.3 Hz, 4H), 3.54 (d, J=11.6 Hz, 3H), 3.62 (d, J=11.9 Hz, 1H), 3.92 (s, 1H), 4.25-4.33 (m, 3H), 4.47 (d, J=12.0 Hz, 1H), 7.14 (d, J=2.5 Hz, 1H), 7.22 (d, J=9.2 Hz, 1H), 7.29-7.38 (m, 2H), 7.44 (t, J=9.0 Hz, 1H), 7.66 (d, J=8.5 Hz, 1H), 7.95 (dd, J=9.3, 5.9 Hz, 1H), 9.02 (s, 1H). m/z (ESI): 868.6 [M+H]⁺.

### Example 91. Synthesis of Compound 118

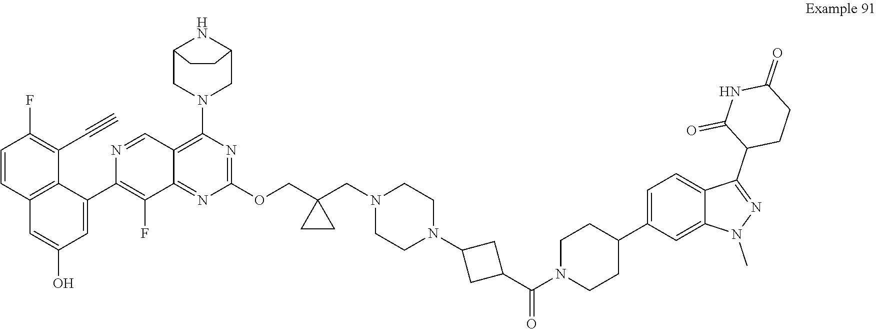 [SMILES: C#Cc1c(F)ccc2cc(O)cc(-c3ncc4c(N5CC6CCC(C5)N6)nc(OCC5(CN6CCN(C7CC(C(=O)N8CCC(c9ccc%10c(C%11CCC(=O)NC%11=O)nn(C)c%10c9)CC8)C7)CC6)CC5)nc4c3F)c12]

Target compound was synthesized according to the procedure of Example 80. ¹H NMR (500 MHz, Methanol-d4) δ 0.45-0.54 (m, 2H), 0.66-0.75 (m, 2H), 1.60-1.72 (m, 2H), 1.74-1.98 (m, 6H), 2.02-2.22 (m, 3H), 2.26-2.56 (m, 10H), 2.66-2.78 (m, 4H), 2.97 (t, J=12.5 Hz, 1H), 3.04-3.22 (m, 2H), 3.38 (s, 1H), 3.62-3.69 (m, 3H), 3.73 (d, J=12.6 Hz, 1H), 4.00 (s, 3H), 4.30-4.39 (m, 2H), 4.52 (t, J=11.4 Hz, 2H), 4.65 (d, J=12.9 Hz, 2H), 7.07 (d, J=8.5 Hz, 1H), 7.21 (s, 1H), 7.32 (dd, J=18.3, 4.6 Hz, 3H), 7.64 (d, J=8.4 Hz, 1H), 7.79-7.89 (m, 1H), 8.99 (s, 1H). m/z (ESI): 1018.7 [M+H]⁺.

### Example 92. Synthesis of Compound 120

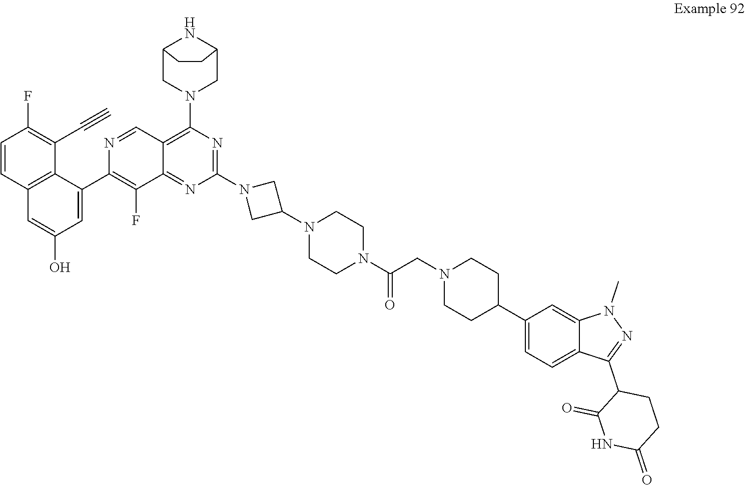 [SMILES: C#Cc1c(F)ccc2cc(O)cc(-c3ncc4c(N5CC6CCC(C5)N6)nc(N5CC(N6CCN(C(=O)CN7CCC(c8ccc9c(C%10CCC(=O)NC%10=O)nn(C)c9c8)CC7)CC6)C5)nc4c3F)c12]

Target compound was synthesized according to the procedure of Example 80. ¹H NMR (500 MHz, Methanol-d4) δ 1.90 (d, J=30.9 Hz, 6H), 2.26-2.38 (m, 3H), 2.51 (s, 3H), 2.59 (s, 2H), 2.73-2.82 (m, 3H), 3.10 (d, J=11.0 Hz, 2H), 3.64 (d, J=7.0 Hz, 4H), 3.78 (s, 2H), 4.03 (s, 3H), 4.15 (s, 2H), 4.30-4.40 (m, 3H), 4.46-4.54 (m, 2H), 4.62 (s, 6H), 7.12 (d, J=8.5 Hz, 1H), 7.22 (d, J=2.5 Hz, 1H), 7.36 (dd, J=11.9, 9.1 Hz, 3H), 7.66 (d, J=8.4 Hz, 1H), 7.88 (dd, J=9.1, 5.7 Hz, 1H), 8.80 (s, 1H). m/z (ESI): 950.7 [M+H]⁺.

### Example 93. Synthesis of Compound 124 Salt

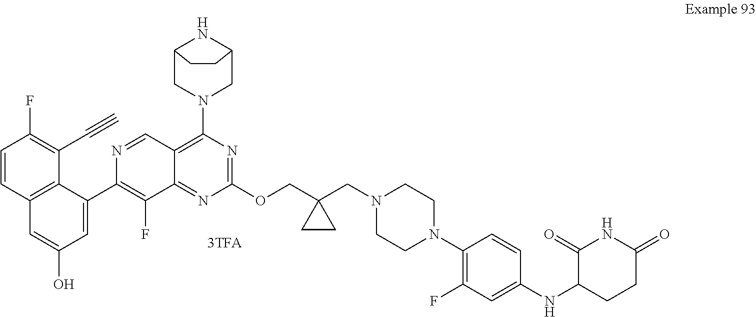 [SMILES: C#Cc1c(F)ccc2cc(O)cc(-c3ncc4c(N5CC6CCC(C5)N6)nc(OCC5(CN6CCN(c7ccc(NC8CCC(=O)NC8=O)cc7F)CC6)CC5)nc4c3F)c12]

Target compound was synthesized according to the procedure of Example 80. ¹H NMR (500 MHz, Methanol-d4) δ 0.94-0.99 (m, 2H), 1.01-1.04 (m, 2H), 1.98 (qd, J=12.4, 4.7 Hz, 1H), 2.14-2.16 (m, 4H), 2.29-2.37 (m, 1H), 2.72-2.80 (m, 1H), 2.80-2.89 (m, 1H), 3.25 (s, 2H), 3.37-3.46 (m, 4H), 3.51 (dd, J=13.6, 4.2 Hz, 1H), 3.90 (d, J=9.5 Hz, 1H), 4.00 (dd, J=28.0, 14.0 Hz, 3H), 4.26-4.31 (m, 3H), 4.52 (d, J=12.0 Hz, 1H), 4.62 (d, J=12.0 Hz, 1H), 4.83-4.90 (m, 4H), 6.47 (d, J=8.6 Hz, 1H), 6.57 (d, J=14.3 Hz, 1H), 6.87 (t, J=9.1 Hz, 1H), 7.26 (s, 1H), 7.36 (t, J=8.9 Hz, 1H), 7.42 (s, 1H), 7.92 (dd, J=8.9, 5.8 Hz, 1H), 9.14 (s, 1H). m/z (ESI): 832.5 [M+H]⁺.

### Example 94. Synthesis of Compound 125 Salt

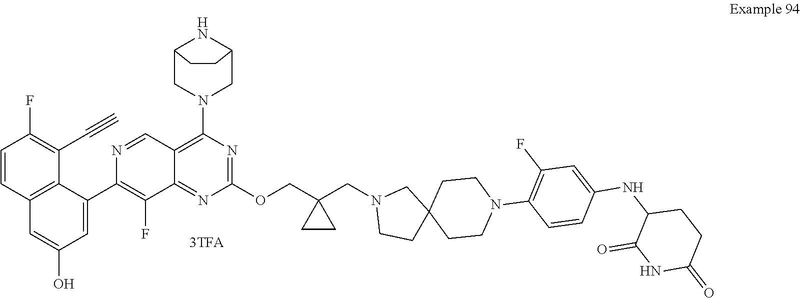 [SMILES: C#Cc1c(F)ccc2cc(O)cc(-c3ncc4c(N5CC6CCC(C5)N6)nc(OCC5(CN6CCC7(CCN(c8ccc(NC9CCC(=O)NC9=O)cc8F)CC7)C6)CC5)nc4c3F)c12]

Target compound was synthesized according to the procedure of Example 80. ¹H NMR (500 MHz, Methanol-d6) δ 0.84-0.95 (m, 2H), 1.09-1.12 (m, 2H), 2.00-2.30 (m, 14H), 2.79-2.89 (m, 2H), 3.21 (d, J=10.8 Hz, 1H), 3.27 (d, J=9.5 Hz, 1H), 3.39-3.43 (m, 1H), 3.50-3.70 (m, 5H), 3.90 (s, 1H), 4.04 (d, J=9.3 Hz, 2H), 4.09-4.20 (m, 1H), 4.24-4.41 (m, 4H), 4.65-4.68 (m, 1H), 5.07 (d, J=10.4 Hz, 1H), 6.51-6.59 (m, 1H), 6.59-6.68 (m, 1H), 7.25 (s, 1H), 7.34 (dd, J=21.9, 8.6 Hz, 2H), 7.40 (d, J=1.6 Hz, 1H), 7.89 (dd, J=8.5, 6.0 Hz, 1H), 9.10 (s, 1H). m/z (ESI): 886.7 [M+H]⁺.

### Example 95. Synthesis of Compound 126 Salt

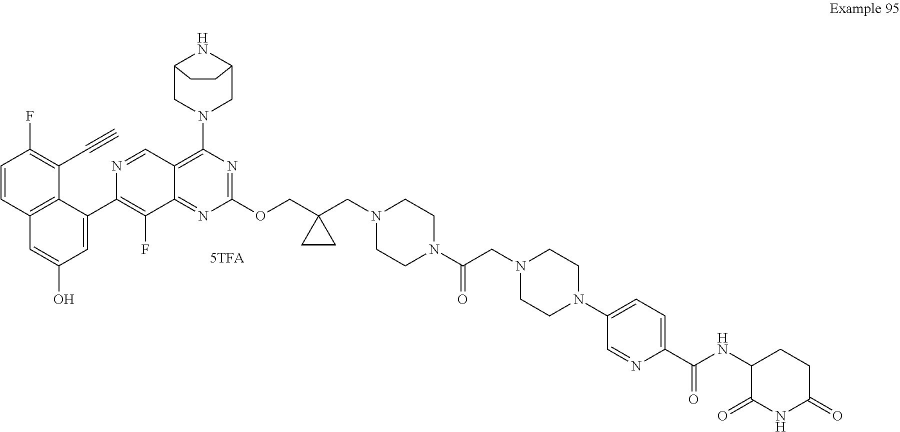 [SMILES: C#Cc1c(F)ccc2cc(O)cc(-c3ncc4c(N5CC6CCC(C5)N6)nc(OCC5(CN6CCN(C(=O)CN7CCN(c8ccc(C(=O)NC9CCC(=O)NC9=O)nc8)CC7)CC6)CC5)nc4c3F)c12]

Target compound was synthesized according to the procedure of Example 80. ¹H NMR (500 MHz, Methanol-d6) δ 0.94 (s, 2H), 1.05-1.06 (m, 2H), 2.15-2.25 (m, 5H), 2.33-2.35 (m, 1H), 2.75 (d, J=16.1 Hz, 1H), 2.82-2.92 (m, 1H), 3.38 (d, J=13.7 Hz, 2H), 3.43 (s, 1H), 3.51 (d, J=13.6 Hz, 2H), 3.57 (s, 5H), 3.75-3.86 (m, 7H), 3.95 (d, J=14.0 Hz, 1H), 4.03 (d, J=13.7 Hz, 1H), 4.31 (d, J=12.5 Hz, 2H), 4.45 (s, 2H), 4.47 (d, J=12.0 Hz, 1H), 4.63 (d, J=11.6 Hz, 1H), 4.82-4.90 (m, 4H), 7.25 (s, 1H), 7.37 (t, J=8.9 Hz, 1H), 7.41 (s, 1H), 7.53 (d, J=8.5 Hz, 1H), 7.91 (dd, J=8.7, 6.1 Hz, 1H), 8.03 (d, J=8.6 Hz, 1H), 8.42 (s, 1H), 9.12 (s, 1H). m/z (ESI): 969.7 [M+H]⁺.

### Example 96. Synthesis of Compound 127 Salt

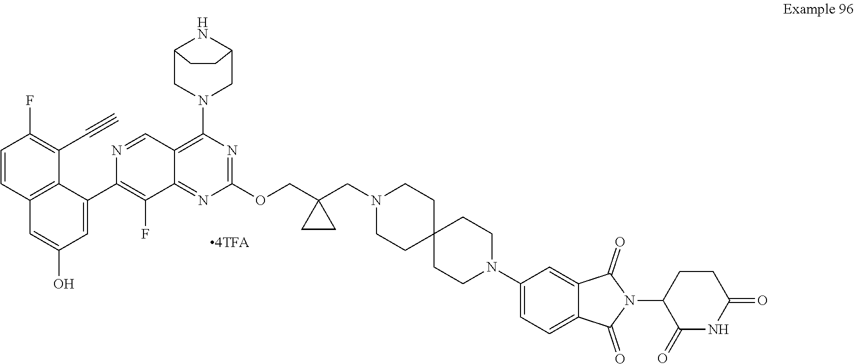 [SMILES: C#Cc1c(F)ccc2cc(O)cc(-c3ncc4c(N5CC6CCC(C5)N6)nc(OCC5(CN6CCC7(CC6)CCN(c6ccc8c(c6)C(=O)N(C6CCC(=O)NC6=O)C8=O)CC7)CC5)nc4c3F)c12]

Target compound was synthesized according to the procedure of Example 80. ¹H NMR (500 MHz, Methanol-d6) δ 0.87-0.97 (m, 2H), 1.00-1.03 (m, 2H), 1.58 (s, 2H), 1.84 (s, 4H), 2.02 (d, J=14.8 Hz, 2H), 2.13-2.22 (m, 5H), 2.69-2.83 (m, 2H), 2.84-2.95 (m, 1H), 3.20 (t, J=12.9 Hz, 2H), 3.27 (d, J=13.7 Hz, 1H), 3.38-3.51 (m, 7H), 3.71 (d, J=12.7 Hz, 1H), 3.80 (d, J=11.9 Hz, 1H), 3.96 (d, J=14.0 Hz, 1H), 4.05 (d, J=13.9 Hz, 1H), 4.31 (d, J=12.9 Hz, 2H), 4.51 (d, J=11.8 Hz, 1H), 4.62 (d, J=12.0 Hz, 1H), 4.82 (s, 2H), 5.10 (dd, J=12.6, 5.4 Hz, 1H), 7.19 (d, J=8.5 Hz, 1H), 7.27 (s, 1H), 7.32 (s, 1H), 7.36 (d, J=8.9 Hz, 1H), 7.43 (s, 1H), 7.69 (d, J=8.5 Hz, 1H), 7.92 (dd, J=8.9, 5.8 Hz, 1H), 9.13 (s, 1H). m/z (ESI): 936.6 [M+H]⁺.

### Example 97. Synthesis of Compound 122

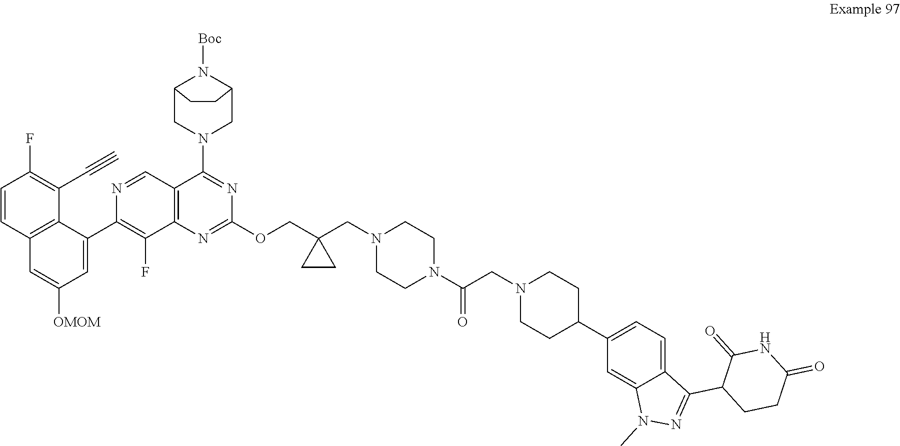

Target compound was synthesized according to the procedure of Example 80. ¹H NMR (500 MHz, DMSO-d6) δ 0.44 (s, 2H), 0.66 (s, 2H), 1.46 (s, 9H), 1.68-1.80 (m, 6H), 1.85 (s, 2H), 2.08-2.19 (m, 3H), 2.30-2.39 (m, 5H), 2.45 (s, 2H), 2.57-2.69 (m, 3H), 2.91 (d, J=10.0 Hz, 2H), 3.14 (s, 2H), 3.41-3.44 (m, 4H), 3.52-3.69 (m, 5H), 3.94 (s, 3H), 3.98 (s, 1H), 4.27-4.34 (m, 5H), 4.56 (d, J=10.0 Hz, 1H), 5.36 (s, 2H), 7.01 (d, J=5.0 Hz, 1H), 7.36 (s, 1H), 7.39 (s, 1H), 7.54 (t, J=10.0 Hz, 1H), 7.59 (d, J=10.0 Hz, 1H), 7.73 (s, 1H), 8.09 (dd, J=10.0, 5.0 Hz, 1H), 9.08 (s, 1H), 10.88 (s, 1H). m/z (ESI): 1122.9 [M+H]⁺.

### Example 98. Synthesis of Compound 128

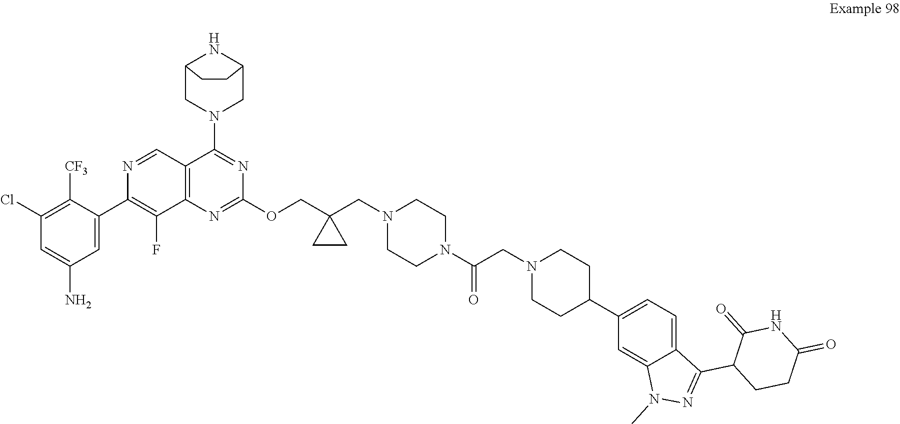 [SMILES: Cn1nc(C2CCC(=O)NC2=O)c2ccc(C3CCN(CC(=O)N4CCN(CC5(COc6nc(N7CC8CCC(C7)N8)c7cnc(-c8cc(N)cc(Cl)c8C(F)(F)F)c(F)c7n6)CC5)CC4)CC3)cc21]

Target compound was synthesized according to the procedure of Example 80. ¹H NMR (500 MHz, DMSO-d6) δ 0.42 (s, 2H), 0.65 (s, 2H), 1.55-1.64 (m, 4H), 1.67-1.80 (m, 4H), 2.10-2.17 (m, 2H), 2.31-2.37 (m, 5H), 2.40-2.48 (m, 4H), 2.57-2.67 (m, 4H), 2.91 (d, J=10.0 Hz, 2H), 3.14 (s, 2H), 3.50-3.61 (m, 6H), 3.95 (s, 3H), 4.29-4.34 (m, 3H), 4.34-4.41 (m, 2H), 6.32 (s, 2H), 6.46 (s, 1H), 6.87 (s, 1H), 7.02 (d, J=5.0 Hz, 1H), 7.40 (s, 1H), 7.59 (d, J=5.0 Hz, 1H), 9.02 (s, 1H), 10.88 (s, 1H). m/z (ESI): 987.7 [M+H]⁺.

### Example 99. Synthesis of Compound 129

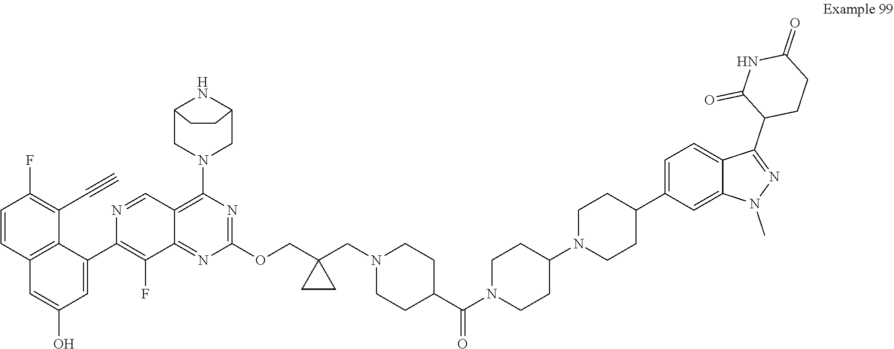 [SMILES: C#Cc1c(F)ccc2cc(O)cc(-c3ncc4c(N5CC6CCC(C5)N6)nc(OCC5(CN6CCC(C(=O)N7CCC(N8CCC(c9ccc%10c(C%11CCC(=O)NC%11=O)nn(C)c%10c9)CC8)CC7)CC6)CC5)nc4c3F)c12]

Target compound was synthesized according to the procedure of Example 80. ¹H NMR (500 MHz, DMSO-d6) δ 0.39 (s, 2H), 0.59-0.66 (m, 2H), 1.20-1.25 (m, 3H), 1.30-1.37 (m, 1H), 1.53 (s, 4H), 1.65 (s, 3H), 1.69-1.83 (m, 6H), 1.86-1.94 (m, 2H), 2.12-2.19 (m, 1H), 2.20-2.28 (m, 3H), 2.31-2.38 (m, 2H), 2.55-2.69 (m, 4H), 2.92-3.01 (m, 5H), 3.51-3.55 (m, 6H), 3.61-3.66 (m, 2H), 3.92-3.97 (m, 5H), 4.22-4.34 (m, 4H), 4.38-4.51 (m, 2H), 7.02 (d, J=10.0 Hz, 1H), 7.18 (s, 1H), 7.37 (s, 1H), 7.40-7.48 (m, 2H), 7.58 (d, J=5.0 Hz, 1H), 7.95 (dd, J=10.0, 5.0 Hz, 1H), 9.01 (s, 1H). m/z (ESI): 1046.9 [M+H]⁺.

### Example 100. Synthesis of Compound 133

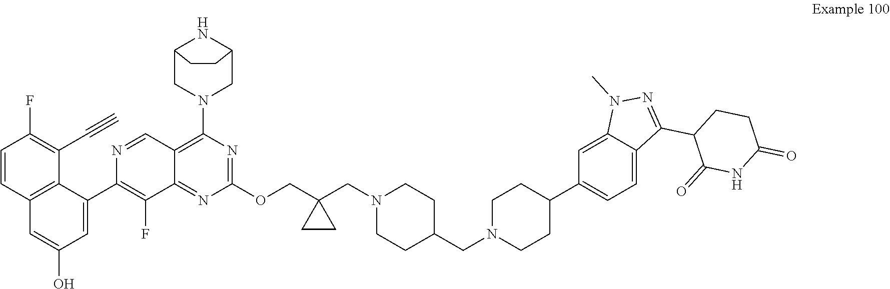 [SMILES: C#Cc1c(F)ccc2cc(O)cc(-c3ncc4c(N5CC6CCC(C5)N6)nc(OCC5(CN6CCC(CN7CCC(c8ccc9c(C%10CCC(=O)NC%10=O)nn(C)c9c8)CC7)CC6)CC5)nc4c3F)c12]

Target compound was synthesized according to the procedure of Example 80. ¹H NMR (500 MHz, Methanol-d4) δ 0.52 (s, 2H), 0.72 (s, 2H), 1.21-1.27 (m, 2H), 1.31-1.34 (m, 2H), 1.38-1.46 (m, 1H), 1.58-1.68 (m, 2H), 1.74-1.80 (m, 3H), 1.86-1.89 (m, 4H), 1.95-2.03 (m, 2H), 2.06-2.13 (m, 2H), 2.24 (d, J=5.0 Hz, 2H), 2.31-2.37 (m, 1H), 2.40-2.53 (m, 3H), 2.66-2.81 (m, 3H), 3.01-3.07 (m, 2H), 3.08-3.15 (m, 2H), 3.21-3.26 (m, 1H), 3.37 (s, 1H), 3.64-3.75 (m, 4H), 4.00 (s, 3H), 4.32-4.41 (m, 2H), 4.44-4.48 (m, 1H), 4.57 (d, J=10.0 Hz, 1H), 4.64 (d, J=15.0 Hz, 1H), 7.08 (d, J=10.0 Hz, 1H), 7.21 (s, 1H), 7.28-7.35 (m, 3H), 7.84 (dd, J=10.0, 5.0 Hz, 1H), 9.00 (s, 1H). m/z (ESI): 949.8 [M+H]⁺.

### Example 101. Synthesis of Compound 134 Salt

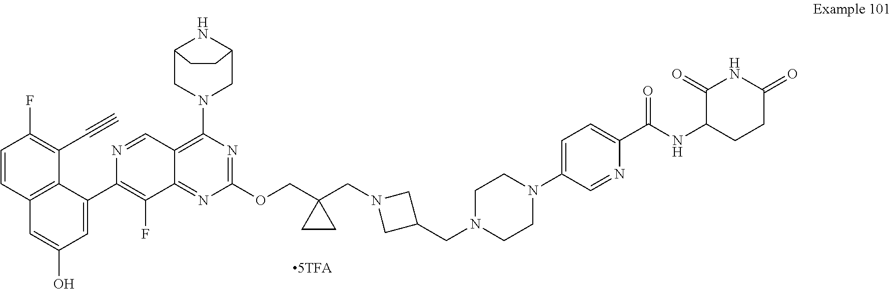 [SMILES: C#Cc1c(F)ccc2cc(O)cc(-c3ncc4c(N5CC6CCC(C5)N6)nc(OCC5(CN6CC(CN7CCN(c8ccc(C(=O)NC9CCC(=O)NC9=O)nc8)CC7)C6)CC5)nc4c3F)c12]

Target compound was synthesized according to the procedure of Example 80. ¹H NMR (500 MHz, Methanol-d4) δ 0.93-0.94 (m, 4H), 2.12-2.19 (m, 5H), 2.33-2.38 (m, 2H), 2.76 (d, J=16.9 Hz, 1H), 2.86-2.91 (m, 1H), 3.48-3.68 (m, 14H), 3.94 (s, 1H), 4.07 (s, 1H), 4.22 (s, 2H), 4.31 (d, J=18.3 Hz, 4H), 4.63 (s, 4H), 7.25-7.38 (m, 5H), 7.87-8.34 (m, 2H), 9.12 (s, 1H). m/z (ESI): 912.7 [M+H]⁺.

### Example 102. Synthesis of Compound 135

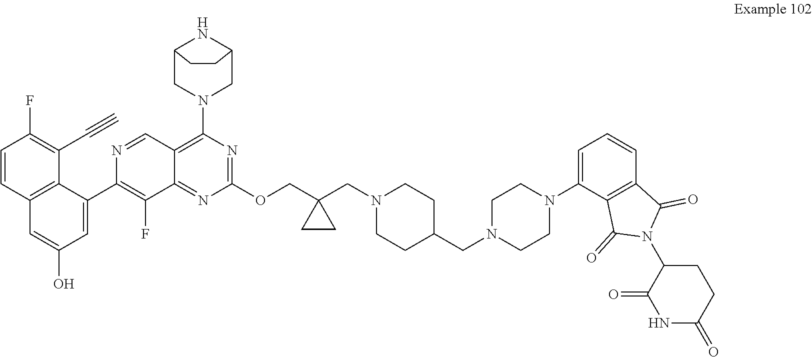 [SMILES: C#Cc1c(F)ccc2cc(O)cc(-c3ncc4c(N5CC6CCC(C5)N6)nc(OCC5(CN6CCC(CN7CCN(c8cccc9c8C(=O)N(C8CCC(=O)NC8=O)C9=O)CC7)CC6)CC5)nc4c3F)c12]

Target compound was synthesized according to the procedure of Example 80. ¹H NMR (500 MHz, Methanol-d4) δ 0.59 (s, 2H), 0.79 (s, 2H), 1.32 (s, 4H), 1.65-1.66 (m, 1H), 1.84 (d, J=13.9 Hz, 4H), 1.89 (s, 2H), 2.15-2.16 (m, 2H), 2.27 (d, J=6.5 Hz, 2H), 2.63 (s, 7H), 2.71-2.76 (m, 2H), 2.86-2.92 (m, 1H), 3.23-3.24 (m, 2H), 3.37 (s, 1H), 3.40 (s, 1H), 3.70 (s, 2H), 3.75 (d, J=13.0 Hz, 2H), 4.41 (d, J=11.1 Hz, 1H), 4.50 (d, J=11.1 Hz, 1H), 4.60 (d, J=12.6 Hz, 1H), 4.67 (d, J=12.2 Hz, 2H), 5.13 (dd, J=12.4, 5.3 Hz, 1H), 7.24 (s, 1H), 7.30-7.36 (m, 2H), 7.37 (s, 1H), 7.40 (d, J=7.1 Hz, 1H), 7.69 (t, J=7.8 Hz, 1H), 7.88 (dd, J=8.8, 5.7 Hz, 1H), 9.04 (s, 1H). m/z (ESI): 965.7 [M+H]⁺.

### Example 103. Synthesis of Compound 136

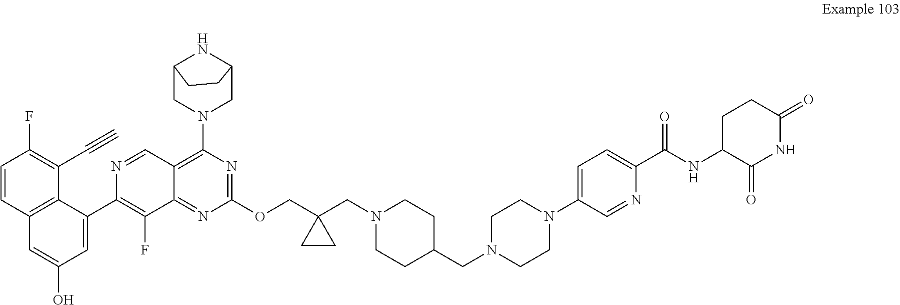 [SMILES: C#Cc1c(F)ccc2cc(O)cc(-c3ncc4c(N5CC6CCC(C5)N6)nc(OCC5(CN6CCC(CN7CCN(c8ccc(C(=O)NC9CCC(=O)NC9=O)nc8)CC7)CC6)CC5)nc4c3F)c12]

Target compound was synthesized according to the procedure of Example 80. ¹H NMR (500 MHz, Methanol-d4) δ 0.52 (d, J=4.8 Hz, 2H), 0.72 (d, J=4.2 Hz, 2H), 1.23 (d, J=11.3 Hz, 2H), 1.29 (s, 4H), 1.63 (d, J=36.9 Hz, 2H), 1.73-1.89 (m, 6H), 1.98 (t, J=11.9 Hz, 2H), 2.22 (d, J=7.2 Hz, 2H), 2.30 (ddd, J=12.1, 5.8, 3.0 Hz, 1H), 2.46 (q, J=12.7 Hz, 2H), 2.56 (t, J=5.0 Hz, 4H), 2.71 (ddd, J=17.8, 4.9, 2.6 Hz, 1H), 2.83 (ddd, J=18.5, 13.5, 5.5 Hz, 1H), 3.10 (t, J=10.7 Hz, 2H), 3.37 (d, J=7.1 Hz, 5H), 3.70 (dd, J=25.7, 11.0 Hz, 4H), 4.38 (d, J=11.0 Hz, 1H), 4.46 (d, J=11.0 Hz, 1H), 4.57 (d, J=12.5 Hz, 1H), 4.64 (d, J=12.6 Hz, 1H), 4.77-4.81 (m, 1H), 7.21 (d, J=2.5 Hz, 1H), 7.29-7.37 (m, 3H), 7.85 (dd, J=9.1, 5.7 Hz, 1H), 7.92 (d, J=8.8 Hz, 1H), 8.29 (d, J=2.8 Hz, 1H), 9.00 (s, 1H). m/z (ESI): 940.7 [M+H]⁺.

### Example 104. Synthesis of Compound 137 Salt

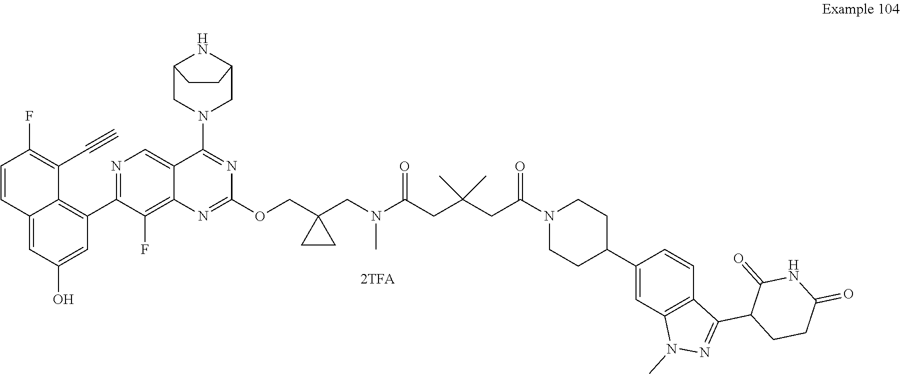 [SMILES: C#Cc1c(F)ccc2cc(O)cc(-c3ncc4c(N5CC6CCC(C5)N6)nc(OCC5(CN(C)C(=O)CC(C)(C)CC(=O)N6CCC(c7ccc8c(C9CCC(=O)NC9=O)nn(C)c8c7)CC6)CC5)nc4c3F)c12]

Target compound was synthesized according to the procedure of Example 80. ¹H NMR (500 MHz, CD₃OD) δ ppm 9.11-8.98 (m, 1H), 7.87-7.75 (m, 1H), 7.64-7.51 (m, 1H), 7.37-7.17 (m, 4H), 7.05-6.90 (m, 1H), 4.81-4.60 (m, 2H), 4.41-4.13 (m, 5H), 4.00-3.87 (m, 5H), 3.60-3.43 (m, 2H), 3.25-3.00 (m, 4H), 2.97-2.29 (m, 12H), 2.15 (d, J=18.0 Hz, 4H), 1.95-1.53 (m, 4H), 1.32 (s, 2H), 1.10 (d, J=9.7 Hz, 5H), 0.88-0.67 (m, 4H). m/z (ESI): 1007.8 [M+H]⁺.

### Example 105. Synthesis of Compound 138

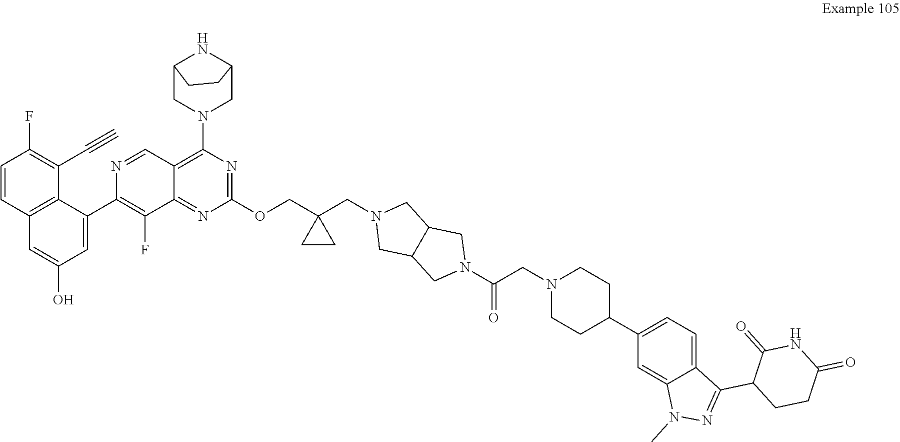 [SMILES: C#Cc1c(F)ccc2cc(O)cc(-c3ncc4c(N5CC6CCC(C5)N6)nc(OCC5(CN6CC7CN(C(=O)CN8CCC(c9ccc%10c(C%11CCC(=O)NC%11=O)nn(C)c%10c9)CC8)CC7C6)CC5)nc4c3F)c12]

Target compound was synthesized according to the procedure of Example 80. ¹H NMR (500 MHz, Methanol-d4) δ 0.49 (s, 2H), 0.69 (s, 2H), 1.31-1.35 (m, 1H), 1.71-1.88 (m, 8H), 2.08-2.21 (m, 2H), 2.27-2.37 (m, 1H), 2.40-2.52 (m, 2H), 2.53-2.61 (m, 3H), 2.64-2.69 (m, 1H), 2.71-2.84 (m, 4H), 2.87-3.03 (m, 3H), 3.09-3.25 (m, 2H), 3.37 (s, 1H), 3.41-3.48 (m, 1H), 3.59-3.69 (m, 6H), 3.72-3.78 (m, 1H), 3.97 (d, J=10.0 Hz, 3H), 4.27-4.39 (m, 2H), 4.46-4.61 (m, 3H), 7.00 (d, J=8.3 Hz, 1H), 7.22 (d, J=2.6 Hz, 1H), 7.24-7.34 (m, 3H), 7.82 (d, J=5.0 Hz, 1H), 8.95 (d, J=15.0 Hz, 1H). m/z (ESI): 1004.8 [M+H]⁺.

### Example 106. Synthesis of Compound 139

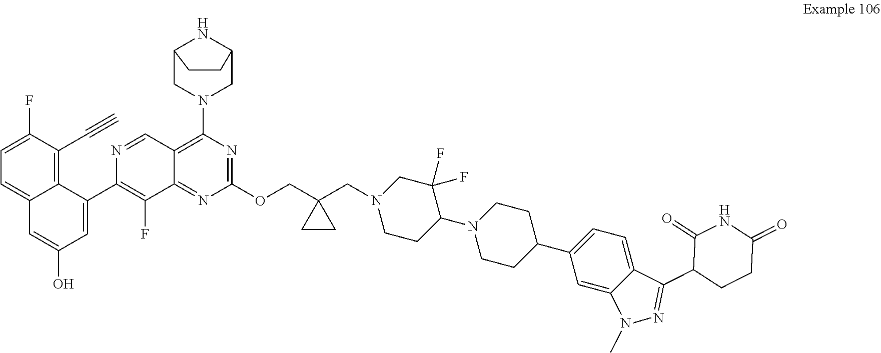 [SMILES: C#Cc1c(F)ccc2cc(O)cc(-c3ncc4c(N5CC6CCC(C5)N6)nc(OCC5(CN6CCC(N7CCC(c8ccc9c(C%10CCC(=O)NC%10=O)nn(C)c9c8)CC7)C(F)(F)C6)CC5)nc4c3F)c12]

Target compound was synthesized according to the procedure of Example 80. ¹H NMR (500 MHz, Methanol-d4) δ 0.49 (m, 2H), 0.68-0.82 (m, 2H), 1.03 (td, J=7.4, 2.6 Hz, 4H), 1.30-1.32 (m, 2H), 1.42 (m, 2H), 1.66 (m, 2H), 1.78-1.94 (m, 6H), 2.07 (m, 1H), 2.16-2.26 (m, 1H), 2.32 (m, 1H), 2.46 (m, 2H), 2.58-2.68 (m, 2H), 2.69-2.82 (m, 3H), 2.99-3.12 (m, 2H), 3.19-3.27 (m, 3H), 3.73 (m, 3H), 4.00 (m, 3H), 4.23 (m, 1H), 4.35 (m, 1H), 4.61 (m, 4H), 4.68-4.82 (m, 2H), 4.86 (m, 2H), 7.06 (m, 1H), 7.16-7.36 (m, 4H), 7.61 (m, 1H), 7.83 (m, 1H), 8.99 (m, 1H). m/z (ESI): 971.7 [M+H]⁺.

### Example 107. Synthesis of Compound 140

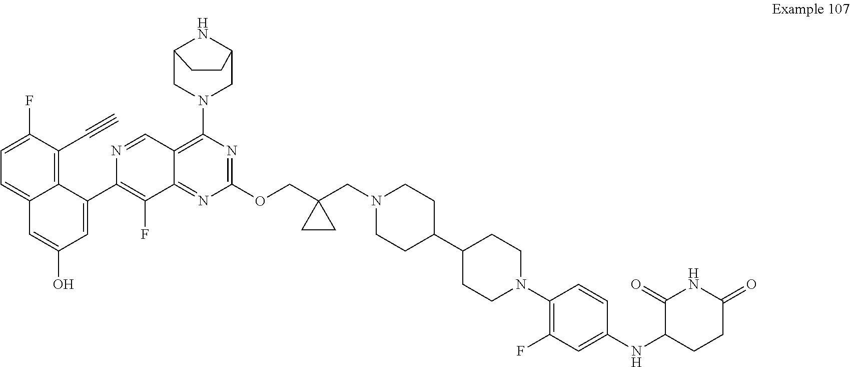 [SMILES: C#Cc1c(F)ccc2cc(O)cc(-c3ncc4c(N5CC6CCC(C5)N6)nc(OCC5(CN6CCC(C7CCN(c8ccc(NC9CCC(=O)NC9=O)cc8F)CC7)CC6)CC5)nc4c3F)c12]

Target compound was synthesized according to the procedure of Example 80. ¹H NMR (500 MHz, Methanol-d4) 30.61 (s, 2H), 0.79 (s, 2H), 1.03 (t, J=7.5 Hz, 1H), 1.30 (m, 4H), 1.37-1.46 (m, 4H), 1.61-1.68 (m, 2H), 1.73-1.76 (m, 2H), 1.80-1.83 (m, 3H), 1.86-1.95 (m, 3H), 2.29-2.39 (m, 2H), 2.52 (m, 2H), 2.65-2.75 (m, 2H), 2.78-2.84 (m, 1H), 3.22 (m, 2H), 3.71 (m, 3H), 4.23 (dd, J=11.8, 4.7 Hz, 1H), 4.36 (m, 1H), 4.58 (m, 6H), 6.41-6.64 (m, 2H), 6.89 (m, 1H), 7.21 (m, 1H), 7.28-7.38 (m, 2H), 7.85 (dd, J=9.1, 5.8 Hz, 1H), 9.02 (s, 1H). m/z (ESI): 914.7 [M+H]⁺.

### Example 108. Synthesis of Compound 141

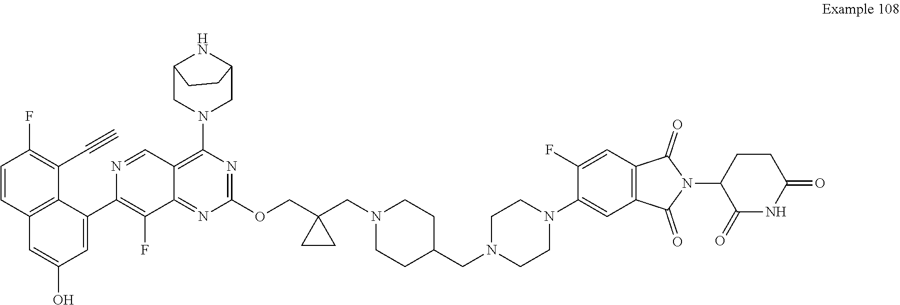 [SMILES: C#Cc1c(F)ccc2cc(O)cc(-c3ncc4c(N5CC6CCC(C5)N6)nc(OCC5(CN6CCC(CN7CCN(c8cc9c(cc8F)C(=O)N(C8CCC(=O)NC8=O)C9=O)CC7)CC6)CC5)nc4c3F)c12]

Target compound was synthesized according to the procedure of Example 80. ¹H NMR (500 MHz, DMSO-d6) δ 0.40 (s, 2H), 0.64 (s, 2H), 0.84 (d, J=7.3 Hz, 1H), 1.06 (d, J=12.4 Hz, 2H), 1.23 (s, 6H), 1.49 (d, J=16.0 Hz, 2H), 1.69 (s, 3H), 1.84 (s, 2H), 1.93-2.08 (m, 2H), 2.14 (d, J=7.1 Hz, 2H), 2.29 (dd, J=13.7, 6.6 Hz, 2H), 2.59-2.67 (m, 1H), 2.82-3.02 (m, 4H), 3.21 (d, J=5.6 Hz, 3H), 3.57-3.68 (m, 4H), 3.94 (d, J=2.5 Hz, 1H), 4.22-4.31 (m, 3H), 4.50 (d, J=12.3 Hz, 1H), 5.10 (d, J=8.6 Hz, 1H), 7.17 (d, J=2.9 Hz, 1H), 7.39 (d, J=2.9 Hz, 1H), 7.41-7.49 (m, 2H), 7.72 (dd, J=11.4, 2.6 Hz, 1H), 7.97 (t, J=7.5 Hz, 1H), 9.03 (s, 1H), 10.17 (s, 1H), 11.11 (s, 1H). m/z (ESI): 984.7 [M+H]⁺.

### Example 109. Synthesis of Compound 142

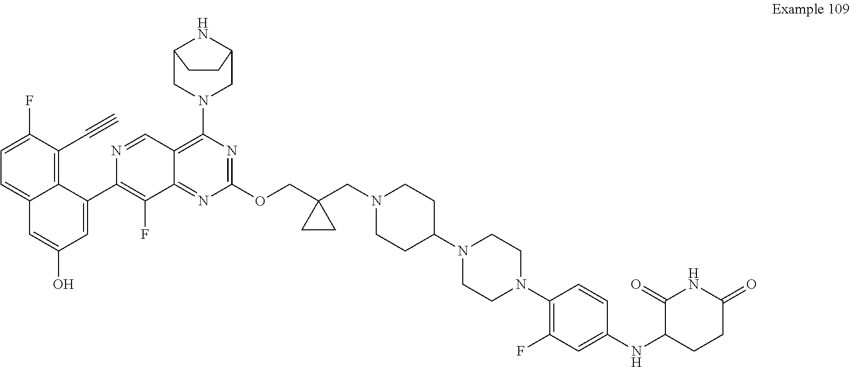 [SMILES: C#Cc1c(F)ccc2cc(O)cc(-c3ncc4c(N5CC6CCC(C5)N6)nc(OCC5(CN6CCC(N7CCN(c8ccc(NC9CCC(=O)NC9=O)cc8F)CC7)CC6)CC5)nc4c3F)c12]

Target compound was synthesized according to the procedure of Example 80. ¹H NMR (500 MHz, Methanol-d4) δ 0.54 (s, 2H), 0.76 (s, 2H), 0.94 (m, 1H), 1.06 (m, 1H), 1.37 (m, 3H), 1.58-1.72 (m, 3H), 1.78-1.96 (m, 6H), 1.98-2.09 (m, 3H), 2.47 (s, 2H), 2.70-2.84 (m, 4H), 2.95-3.01 (m, 4H), 3.22 (m, 2H), 3.67-3.78 (m, 4H), 4.37-4.54 (m, 2H), 6.22-6.47 (m, 1H), 6.50-6.67 (m, 1H), 6.90 (m, 1H), 7.24 (m, 1H), 7.32-7.47 (m, 2H), 7.88 (dd, J=9.1, 5.7 Hz, 1H), 9.03 (s, 1H). m/z (ESI): 915.7 [M+H]⁺.

### Example 110. Synthesis of Compound 143

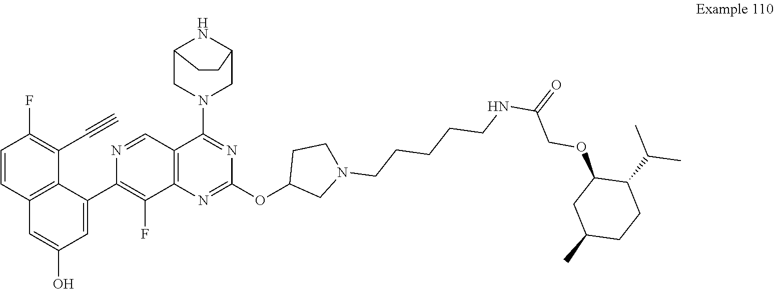 [SMILES: C#Cc1c(F)ccc2cc(O)cc(-c3ncc4c(N5CC6CCC(C5)N6)nc(OC5CCN(CCCCCNC(=O)CO[C@@H]6C[C@H](C)CC[C@H]6C(C)C)C5)nc4c3F)c12]

Target compound was synthesized according to the procedure of Example 4. ¹H NMR (500 MHz, Methanol-d4) δ 0.83 (dd, J=7.0, 1.6 Hz, 3H), 0.87-0.93 (m, 2H), 0.95 (dd, J=6.7, 2.6 Hz, 6H), 1.06 (m, 2H), 1.43 (m, 4H), 1.60 (m, 4H), 1.70 (m, 4H), 1.79-2.00 (m, 4H), 2.11 (d, J=12.4 Hz, 2H), 2.24 (m, 1H), 2.35-2.46 (m, 1H), 2.55 (m, 2H), 2.65 (m, 1H), 2.83-2.91 (m, 2H), 3.27 (m, 3H), 3.40 (s, 1H), 3.73 (m, 4H), 3.90 (m, 1H), 4.06 (m, 1H), 4.59 (d, J=12.6 Hz, 1H), 4.67 (d, J=12.6 Hz, 1H), 5.45-5.71 (m, 1H), 7.23 (m, 1H), 7.32-7.41 (m, 2H), 7.87 (dd, J=9.1, 5.6 Hz, 1H), 9.03 (s, 1H). m/z (ESI): 810.8 [M+H]⁺.

### Example 111. Synthesis of Compound 144 Salt

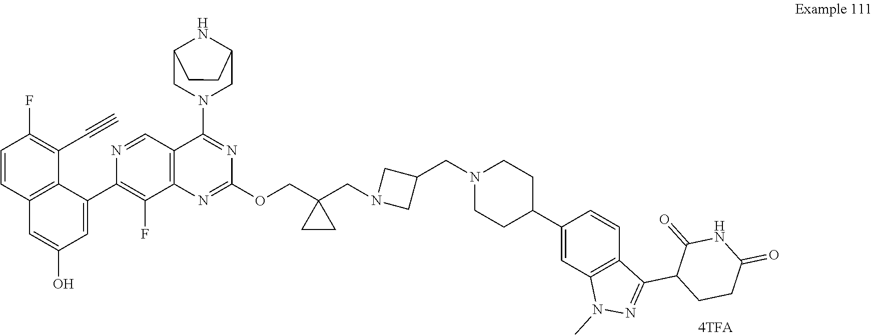 [SMILES: C#Cc1c(F)ccc2cc(O)cc(-c3ncc4c(N5CC6CCC(C5)N6)nc(OCC5(CN6CC(CN7CCC(c8ccc9c(C%10CCC(=O)NC%10=O)nn(C)c9c8)CC7)C6)CC5)nc4c3F)c12]

Target compound was synthesized according to the procedure of Example 80. ¹H NMR (500 MHz, CD3OD) δ ppm 9.09 (s, 1H), 7.72 (s, 1H), 7.64 (s, 1H), 7.49-7.06 (m, 4H), 7.05-6.78 (m, 1H), 4.85-4.43 (m, 2H), 4.43-4.11 (m, 4H), 4.10-3.78 (m, 4H), 3.73-3.38 (m, 6H), 3.24-3.08 (m, 1H), 3.01-2.67 (m, 2H), 2.54-2.42 (m, 1H), 2.40-1.94 (m, 7H), 1.67-1.55 (m, 1H), 1.41-1.11 (m, 11H), 1.05-0.80 (m, 4H). m/z (ESI): 921.7 [M+H]⁺.

### Example 112. Synthesis of Compound 145

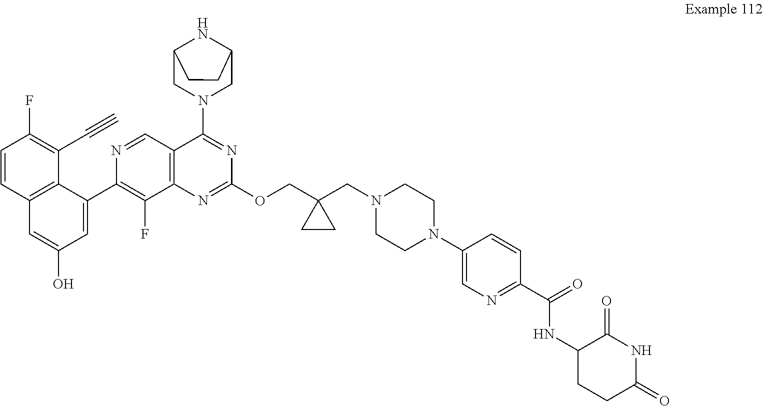 [SMILES: C#Cc1c(F)ccc2cc(O)cc(-c3ncc4c(N5CC6CCC(C5)N6)nc(OCC5(CN6CCN(c7ccc(C(=O)NC8CCC(=O)NC8=O)nc7)CC6)CC5)nc4c3F)c12]

Target compound was synthesized according to the procedure of Example 80. ¹H NMR (500 MHz, CD3OD) δ ppm 9.00 (s, 1H), 8.27 (s, 1H), 8.00-7.79 (m, 2H), 7.43-7.27 (m, 3H), 7.18 (s, 1H), 4.79-4.62 (m, 2H), 4.60-4.50 (m, 1H), 4.48-4.37 (m, 1H), 4.18-3.93 (m, 2H), 3.82 (s, 2H), 3.69-3.42 (m, 1H), 3.36 (s, 4H), 2.87-2.69 (m, 5H), 2.62-2.45 (m, 2H), 2.40-2.10 (m, 2H), 2.06-1.89 (m, 4H), 1.62 (s, 1H), 0.90 (s, 1H), 0.76 (s, 2H), 0.56 (s, 2H). m/z (ESI): 843.7 [M+H]⁺.

### Example 113. Synthesis of Compound 146 Salt

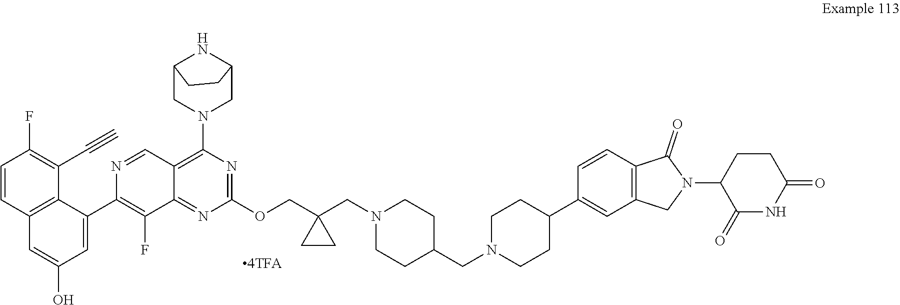 [SMILES: C#Cc1c(F)ccc2cc(O)cc(-c3ncc4c(N5CC6CCC(C5)N6)nc(OCC5(CN6CCC(CN7CCC(c8ccc9c(c8)CN(C8CCC(=O)NC8=O)C9=O)CC7)CC6)CC5)nc4c3F)c12]

Target compound was synthesized according to the procedure of Example 80. ¹H NMR (500 MHz, Methanol-d4) δ 0.90-0.97 (m, 2H), 1.03-1.07 (m, 2H), 1.32 (s, 4H), 1.81 (s, 2H), 2.15-2.20 (m, 11H), 2.53 (dt, J=13.1, 8.8 Hz, 1H), 2.81-2.85 (m, 1H), 2.90-3.00 (m, 1H), 3.09-3.15 (m, 6H), 3.26 (d, J=12.1 Hz, 1H), 3.47 (d, J=13.3 Hz, 1H), 3.52 (s, 1H), 3.75 (s, 2H), 3.92-3.97 (m, 2H), 4.06-4.08 (m, 2H), 4.32 (d, J=14.2 Hz, 2H), 4.41-4.48 (m, 1H), 4.49-4.57 (m, 2H), 4.65 (d, J=11.3 Hz, 1H), 5.18 (dd, J=13.2, 4.9 Hz, 1H), 7.27 (s, 1H), 7.34-7.43 (m, 2H), 7.48 (d, J=7.3 Hz, 1H), 7.54 (s, 1H), 7.81 (d, J=7.8 Hz, 1H), 7.87-7.96 (m, 1H), 9.12 (s, 1H). m/z (ESI): 950.8 [M+H]⁺.

### Example 114. Synthesis of Compound 147

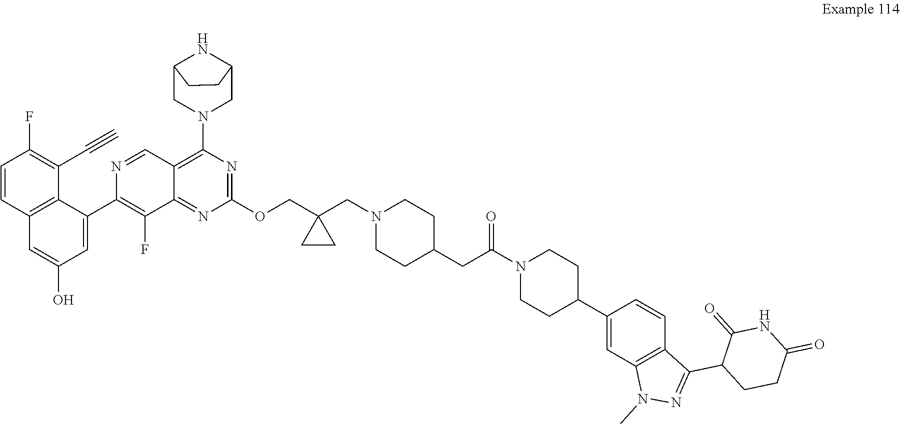 [SMILES: C#Cc1c(F)ccc2cc(O)cc(-c3ncc4c(N5CC6CCC(C5)N6)nc(OCC5(CN6CCC(CC(=O)N7CCC(c8ccc9c(C%10CCC(=O)NC%10=O)nn(C)c9c8)CC7)CC6)CC5)nc4c3F)c12]

Target compound was synthesized according to the procedure of Example 80. ¹H NMR (500 MHz, Methanol-d4) δ 0.77 (s, 2H), 0.93 (s, 2H), 1.32 (s, 2H), 1.54-1.70 (m, 4H), 1.81-2.10 (m, 10H), 2.36-2.41 (m, 3H), 2.45-2.53 (m, 1H), 2.74-2.84 (m, 5H), 2.95-3.05 (m, 3H), 3.15-3.26 (m, 1H), 3.42 (s, 1H), 3.55-3.65 (m, 2H), 3.77 (s, 4H), 4.00-4.10 (m, 4H), 4.38-4.42 (m, 1H), 4.43-4.56 (m, 2H), 7.05 (d, J=8.3 Hz, 1H), 7.23 (s, 1H), 7.32 (s, 2H), 7.66 (d, J=8.1 Hz, 1H), 7.80-7.85 (m, 1H), 8.56 (s, 1H), 9.07 (s, 1H). m/z (ESI): 977.8 [M+H]⁺.

### Example 115. Synthesis of Compound 148

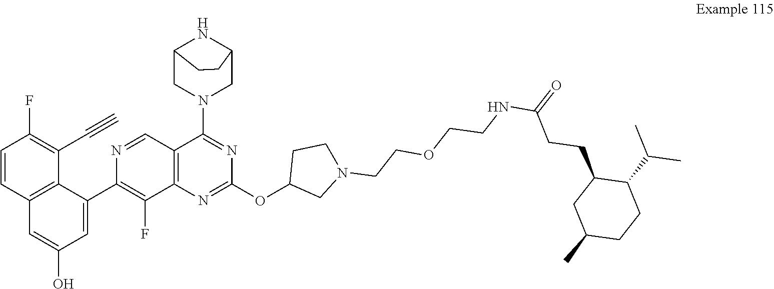 [SMILES: C#Cc1c(F)ccc2cc(O)cc(-c3ncc4c(N5CC6CCC(C5)N6)nc(OC5CCN(CCOCCNC(=O)CC[C@@H]6C[C@H](C)CC[C@H]6C(C)C)C5)nc4c3F)c12]

Target compound was synthesized according to the procedure of Example 110. ¹H NMR (500 MHz, Methanol-d4) δ 0.76-0.80 (m, 3H), 0.87-0.92 (m, 6H), 1.03 (t, J=5.0 Hz, 3H), 1.38-1.45 (m, 2H), 1.59-1.69 (m, 4H), 1.76-1.82 (m, 1H), 1.84-1.91 (m, 2H), 2.03-2.11 (m, 2H), 2.15-2.22 (m, 1H), 2.39-2.46 (m, 1H), 2.67-2.73 (m, 1H), 2.75-2.80 (m, 2H), 2.87-2.96 (m, 2H), 3.15-3.27 (m, 4H), 3.37 (s, 1H), 3.41-3.47 (m, 2H), 3.51-3.58 (m, 2H), 3.60-3.69 (m, 4H), 3.83-3.91 (m, 1H), 4.00-4.09 (m, 1H), 5.55 (s, 1H), 7.20 (s, 1H), 7.29-7.37 (m, 2H), 7.86 (dd, J=10.0, 5.0 Hz, 1H), 9.01 (s, 1H). m/z (ESI): 812.7 [M+H]⁺.

### Example 116. Synthesis of Compound 149

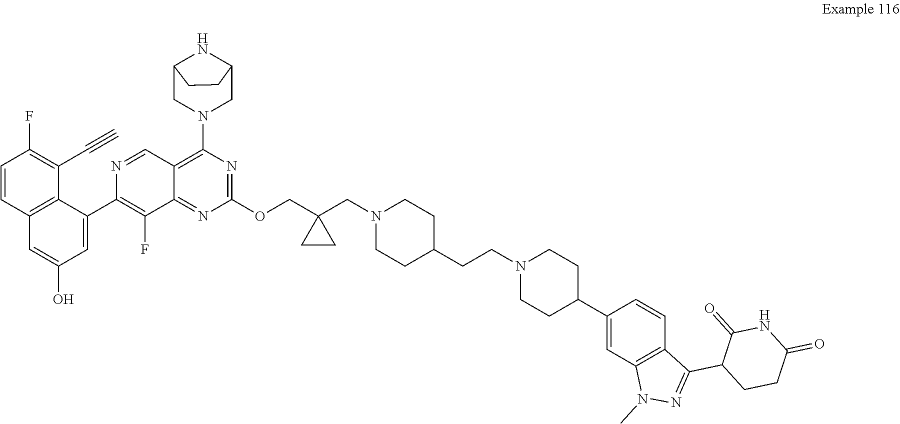 [SMILES: C#Cc1c(F)ccc2cc(O)cc(-c3ncc4c(N5CC6CCC(C5)N6)nc(OCC5(CN6CCC(CCN7CCC(c8ccc9c(C%10CCC(=O)NC%10=O)nn(C)c9c8)CC7)CC6)CC5)nc4c3F)c12]

Target compound was synthesized according to the procedure of Example 80. ¹H NMR (500 MHz, Methanol-d4) δ 0.83 (s, 2H), 0.97 (s, 2H), 1.32 (s, 1H), 1.69-1.75 (m, 5H), 1.86-2.21 (m, 11H), 2.32-2.38 (m, 1H), 2.48-2.53 (m, 1H), 2.75-2.91 (m, 6H), 3.03 (s, 2H), 3.13 (d, J=15.0 Hz, 1H), 3.47 (s, 1H), 3.52 (d, J=10.5 Hz, 2H), 3.70-3.74 (m, 2H), 3.86 (t, J=12.0 Hz, 2H), 3.96 (s, 2H), 4.06 (s, 3H), 4.40 (dd, J=8.7, 4.9 Hz, 1H), 4.47 (d, J=11.7 Hz, 1H), 4.56 (d, J=11.6 Hz, 1H), 4.70-4.74 (m, 2H), 7.11 (d, J=8.3 Hz, 1H), 7.25 (s, 1H), 7.34-7.37 (m, 2H), 7.40 (s, 1H), 7.73 (d, J=8.3 Hz, 1H), 7.84-7.91 (m, 1H), 9.10 (s, 1H). m/z (ESI): 963.8 [M+H]⁺.

### Example 117. Synthesis of Compound 150 Salt

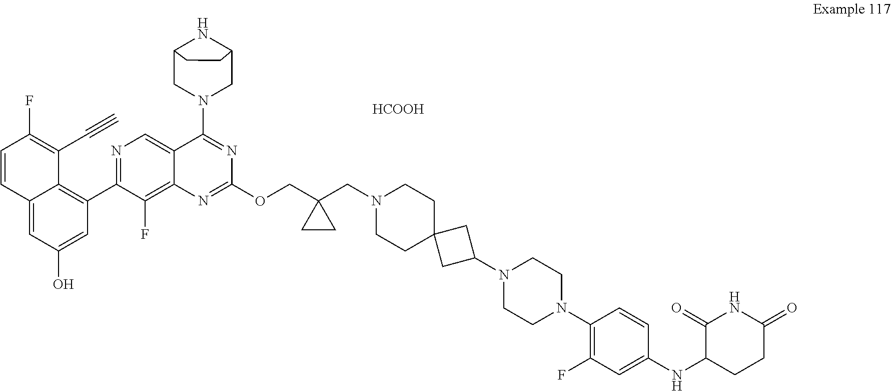 [SMILES: C#Cc1c(F)ccc2cc(O)cc(-c3ncc4c(N5CC6CCC(C5)N6)nc(OCC5(CN6CCC7(CC6)CC(N6CCN(c8ccc(NC9CCC(=O)NC9=O)cc8F)CC6)C7)CC5)nc4c3F)c12 ; "HCOOH"]

Target compound was synthesized according to the procedure of Example 80. ¹H NMR (500 MHz, CD₃OD) δ 9.05 (s, 1H), 8.46 (s, 1H), 7.87 (dd, J=9.2, 5.7 Hz, 1H), 7.37-7.29 (m, 2H), 7.20 (d, J=2.7 Hz, 1H), 6.90 (t, J=9.0 Hz, 1H), 6.58-6.42 (m, 2H), 4.71-4.52 (m, 3H), 4.35 (d, J=11.8 Hz, 1H), 4.23 (dd, J=11.8, 4.8 Hz, 1H), 3.97-3.71 (m, 4H), 3.46-3.35 (m, 2H), 3.21-2.88 (m, 8H), 2.86-2.65 (m, 3H), 2.65-2.47 (m, 3H), 2.38-2.23 (m, 1H), 2.19-2.00 (m, 2H), 1.99-1.81 (m, 9H), 1.80-1.67 (m, 2H), 1.36-1.23 (m, 2H), 0.93 (s, 2H), 0.79 (s, 2H). m/z (ESI): 955.6 [M+H]⁺.

### Example 118. Synthesis of Compound 151 Salt

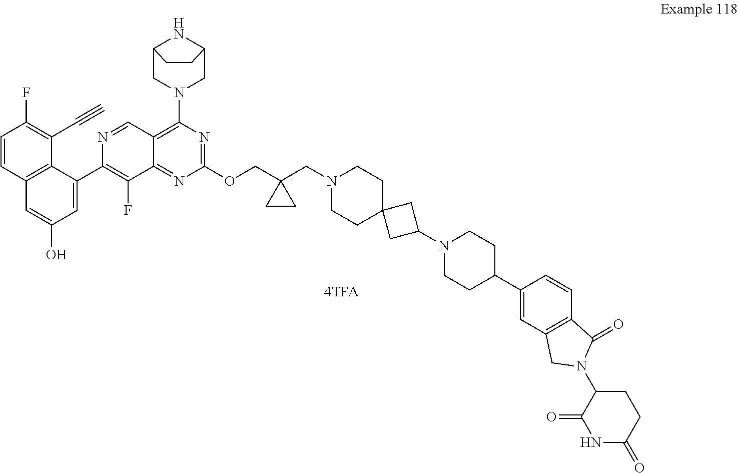 [SMILES: C#Cc1c(F)ccc2cc(O)cc(-c3ncc4c(N5CC6CCC(C5)N6)nc(OCC5(CN6CCC7(CC6)CC(N6CCC(c8ccc9c(c8)CN(C8CCC(=O)NC8=O)C9=O)CC6)C7)CC5)nc4c3F)c12]

Target compound was synthesized according to the procedure of Example 80. ¹H NMR (500 MHz, Methanol-d4) δ 0.91 (d, J=6.2 Hz, 2H), 1.02 (d, J=10.3 Hz, 2H), 1.29 (d, J=4.3 Hz, 9H), 1.37 (dd, J=6.7, 4.2 Hz, 3H), 1.61 (d, J=6.9 Hz, 1H), 2.03 (d, J=6.3 Hz, 4H), 2.17 (dd, J=16.2, 8.4 Hz, 6H), 2.35 (t, J=7.4 Hz, 1H), 2.47-2.53 (m, 1H), 2.79 (d, J=17.6 Hz, 1H), 2.92-2.96 (m, 2H), 3.19-3.25 (m, 2H), 3.41-3.45 (m, 2H), 3.53-3.59 (m, 1H), 3.73 (dd, J=11.9, 7.0 Hz, 2H), 3.89 (d, J=17.0 Hz, 2H), 4.01 (s, 1H), 4.29 (d, J=13.9 Hz, 2H), 4.52 (d, J=11.2 Hz, 2H), 4.78-4.85 (m, 2H), 5.17 (dd, J=13.2, 5.4 Hz, 1H), 7.22 (d, J=2.6 Hz, 1H), 7.34-7.39 (m, 2H), 7.65 (d, J=8.1 Hz, 1H), 7.70 (s, 1H), 7.81 (t, J=7.7 Hz, 1H), 7.89 (dd, J=9.2, 5.5 Hz, 1H), 9.08 (s, 1H). m/z (ESI): 975.46 [M+H]⁺.

### Example 119. Synthesis of Compound 152 Salt

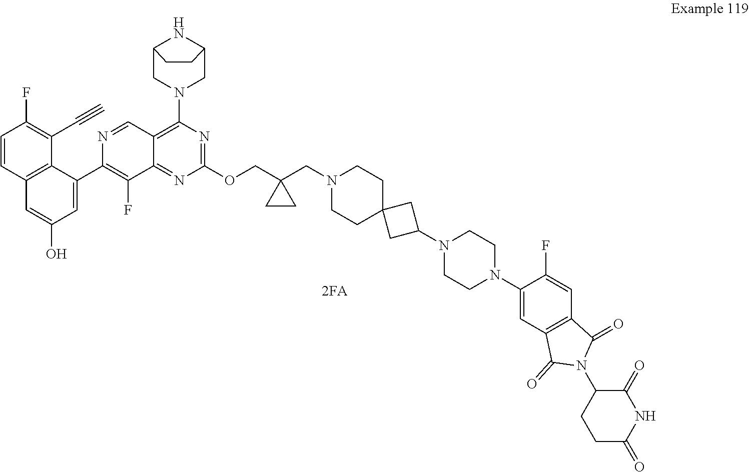 [SMILES: C#Cc1c(F)ccc2cc(O)cc(-c3ncc4c(N5CC6CCC(C5)N6)nc(OCC5(CN6CCC7(CC6)CC(N6CCN(c8cc9c(cc8F)C(=O)N(C8CCC(=O)NC8=O)C9=O)CC6)C7)CC5)nc4c3F)c12]

Target compound was synthesized according to the procedure of Example 80. ¹H NMR (500 MHz, DMSO-d6) δ 0.41 (s, 2H), 0.64 (s, 2H), 1.23 (s, 6H), 1.39-1.58 (m, 6H), 1.73 (s, 4H), 1.88 (d, J=9.3 Hz, 2H), 1.95-2.08 (m, 2H), 2.24-2.42 (m, 9H), 2.56-2.73 (m, 3H), 2.87 (t, J=8.7 Hz, 2H), 3.93 (s, 2H), 4.21-4.37 (m, 3H), 4.52 (d, J=12.5 Hz, 1H), 5.10 (dd, J=12.9, 5.5 Hz, 1H), 7.17 (s, 1H), 7.36-7.52 (m, 3H), 7.72 (d, J=11.4 Hz, 1H), 7.97 (dd, J=9.1, 6.0 Hz, 1H), 8.22 (s, 2H), 9.04 (s, 1H), 11.12 (s, 1H). m/z (ESI): 1008.43 [M+H]⁺.

### Example 120. Synthesis of Compound 153 Salt

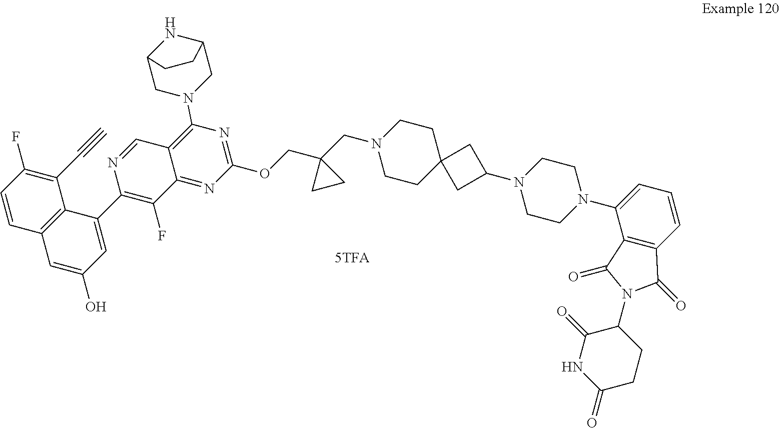 [SMILES: C#Cc1c(F)ccc2cc(O)cc(-c3ncc4c(N5CC6CCC(C5)N6)nc(OCC5(CN6CCC7(CC6)CC(N6CCN(c8cccc9c8C(=O)N(C8CCC(=O)NC8=O)C9=O)CC6)C7)CC5)nc4c3F)c12]

Target compound was synthesized according to the procedure of Example 80. ¹H NMR (500 MHz, Methanol-d4) δ 0.83-1.11 (m, 4H), 1.90-1.99 (m, 1H), 2.02-2.22 (m, 8H), 2.27 (m, 2H), 2.48-2.60 (m, 1H), 2.72 (m, 2H), 2.81-2.95 (m, 1H), 2.97-3.11 (m, 2H), 3.14-3.28 (m, 4H), 3.42 (m, 2H), 3.57 (m, 2H), 3.71-3.79 (m, 1H), 3.81-4.05 (m, 6H), 4.29 (d, J=14.6 Hz, 2H), 4.42-4.68 (m, 2H), 4.76-4.86 (m, 4H), 5.10-5.22 (m, 1H), 7.23 (s, 1H), 7.32-7.44 (m, 3H), 7.51 (d, J=7.2 Hz, 1H), 7.76 (m, 1H), 7.89 (m, 1H), 9.09 (s, 1H). m/z (ESI): 991.8 [M+H]⁺.

### Example 121. Synthesis of Compound 154 Salt

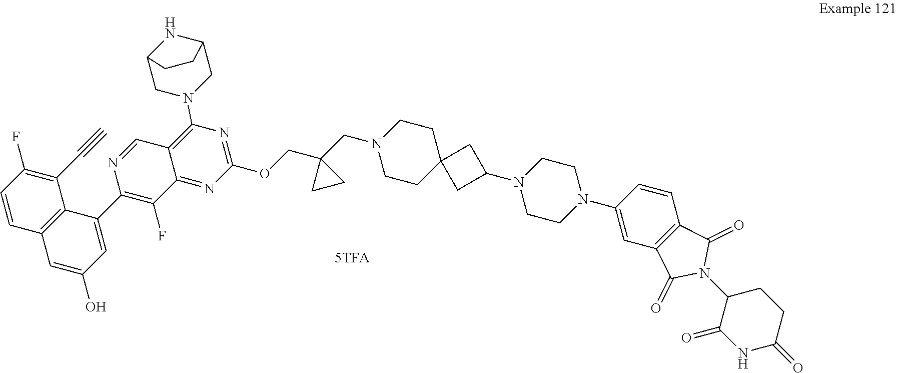 [SMILES: C#Cc1c(F)ccc2cc(O)cc(-c3ncc4c(N5CC6CCC(C5)N6)nc(OCC5(CN6CCC7(CC6)CC(N6CCN(c8ccc9c(c8)C(=O)N(C8CCC(=O)NC8=O)C9=O)CC6)C7)CC5)nc4c3F)c12]

Target compound was synthesized according to the procedure of Example 80. ¹H NMR (500 MHz, Methanol-d4) δ 0.89 (m, 2H), 1.02 (m, 2H), 1.96 (m, 1H), 2.02-2.25 (m, 9H), 2.27 (d, J=8.8 Hz, 2H), 2.54 (m, 1H), 2.66-2.80 (m, 2H), 2.88 (m, 1H), 3.04 (m, 2H), 3.23 (m, 4H), 3.40 (m, 4H), 3.72-4.04 (m, 4H), 4.29 (m, 2H), 4.45 (m, 1H), 4.56 (m, 1H), 4.74-4.87 (m, 6H), 5.10 (dd, J=12.6, 5.4 Hz, 1H), 7.23 (m, 1H), 7.32-7.43 (m, 3H), 7.47 (s, 1H), 7.76 (d, J=8.5 Hz, 1H), 7.89 (m, 1H), 9.09 (s, 1H). m/z (ESI): 991.8 [M+H]⁺.

### Example 122. Synthesis of Compound 155 Salt

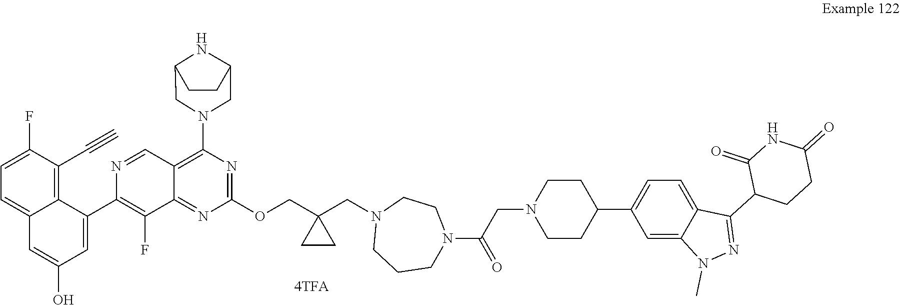 [SMILES: C#Cc1c(F)ccc2cc(O)cc(-c3ncc4c(N5CC6CCC(C5)N6)nc(OCC5(CN6CCCN(C(=O)CN7CCC(c8ccc9c(C%10CCC(=O)NC%10=O)nn(C)c9c8)CC7)CC6)CC5)nc4c3F)c12]

Target compound was synthesized according to the procedure of Example 80. ¹H NMR (500 MHz, Methanol-d4) δ 0.83-1.06 (m, 4H), 1.97-2.23 (m, 9H), 2.34 (m, 2H), 2.49 (m, 2H), 2.71-2.88 (m, 2H), 2.98-3.26 (m, 4H), 3.46 (m, 4H), 3.58-3.69 (m, 2H), 3.77 (m, 2H), 3.87-4.08 (m, 7H), 4.29 (m, 5H), 4.37-4.42 (m, 2H), 4.56 (m, 1H), 4.84 (m, 2H), 7.11 (m, 1H), 7.19-7.46 (m, 4H), 7.71 (m, 1H), 7.84 (s, 1H), 9.08 (s, 1H). m/z (ESI): 992.9 [M+H]⁺.

### Example 123. Synthesis of Compound 156 Salt

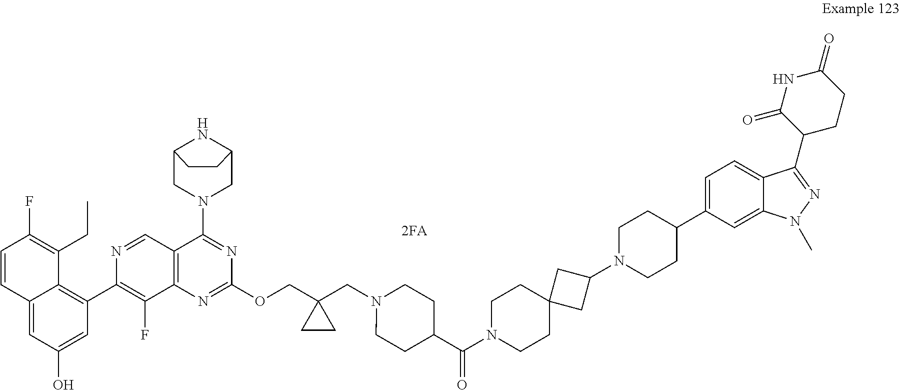 [SMILES: CCc1c(F)ccc2cc(O)cc(-c3ncc4c(N5CC6CCC(C5)N6)nc(OCC5(CN6CCC(C(=O)N7CCC8(CC7)CC(N7CCC(c9ccc%10c(C%11CCC(=O)NC%11=O)nn(C)c%10c9)CC7)C8)CC6)CC5)nc4c3F)c12]

Target compound was synthesized according to the procedure of Example 80. ¹H NMR (500 MHz, Methanol-d4) δ 0.74-0.83 (m, 5H), 0.88-0.95 (m, 2H), 1.57-1.73 (m, 4H), 1.88-2.23 (m, 16H), 2.27-2.36 (m, 3H), 2.43-2.52 (m, 2H), 2.69-2.82 (m, 5H), 2.93-3.13 (m, 4H), 3.44-3.55 (m, 5H), 3.55-3.69 (m, 4H), 3.79-3.91 (m, 2H), 3.94-4.04 (m, 5H), 4.36 (dd, J=10.0, 5.0 Hz, 1H), 4.42-4.52 (m, 2H), 4.67-4.79 (m, 2H), 7.06 (s, 1H), 7.11 (d, J=10.0 Hz, 1H), 7.26 (d, J=10.0 Hz, 1H), 7.32 (s, 1H), 7.39 (s, 1H), 7.66-7.73 (m, 2H), 8.52 (s, 1H), 9.10 (s, 1H). m/z (ESI): 1090.8 [M+H]⁺.

### Example 124. Synthesis of Compound 157 Salt

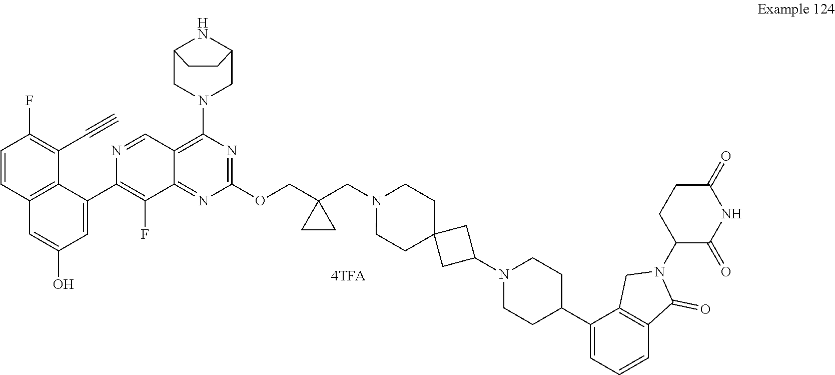 [SMILES: C#Cc1c(F)ccc2cc(O)cc(-c3ncc4c(N5CC6CCC(C5)N6)nc(OCC5(CN6CCC7(CC6)CC(N6CCC(c8cccc9c8CN(C8CCC(=O)NC8=O)C9=O)CC6)C7)CC5)nc4c3F)c12]

Target compound was synthesized according to the procedure of Example 80. ¹H NMR (500 MHz, Methanol-d4) δ 0.89 (m, 2H), 1.01 (m, 2H), 2.00-2.27 (m, 14H), 2.52 (m, 2H), 2.75-2.86 (m, 1H), 2.89-3.10 (m, 6H), 3.23 (m, 1H), 3.40 (m, 2H), 3.58 (m, 2H), 3.73 (m, 2H), 3.82-4.07 (m, 3H), 4.29 (d, J=13.7 Hz, 2H), 4.42-4.66 (m, 4H), 4.75-4.87 (m, 4H), 5.20 (m, 1H), 7.23 (d, J=2.6 Hz, 1H), 7.31-7.41 (m, 2H), 7.56 (m, 2H), 7.70-7.76 (m, 1H), 7.87 (m, 1H), 9.08 (s, 1H). m/z (ESI): 976.6 [M+H]⁺.

### Example 125. Synthesis of Compound 158 Salt

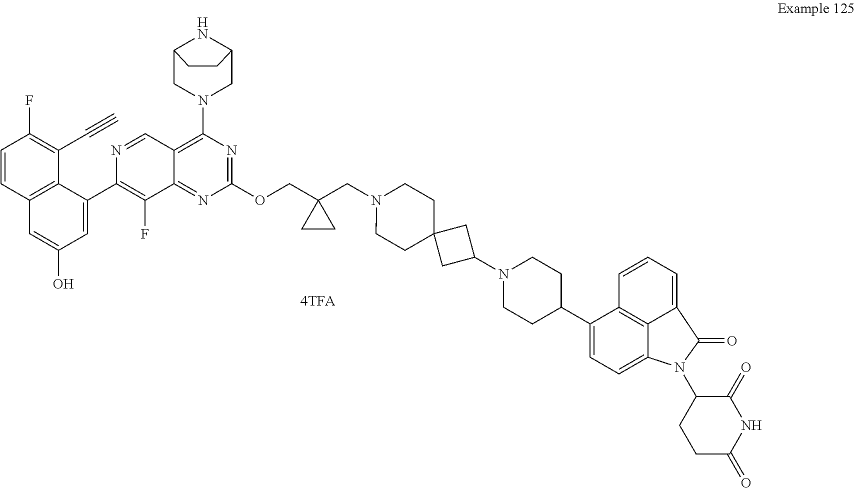 [SMILES: C#Cc1c(F)ccc2cc(O)cc(-c3ncc4c(N5CC6CCC(C5)N6)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12]

Target compound was synthesized according to the procedure of Example 80. ¹H NMR (500 MHz, Methanol-d4) δ 0.93 (m, 2H), 1.05 (m, 2H), 1.40 (m, J=6.8, 4.3 Hz, 4H), 1.96-2.36 (m, 13H), 2.62 (m, 1H), 2.82-2.94 (m, 2H), 2.97-3.15 (m, 4H), 3.22-3.32 (m, 2H), 3.44 (d, J=24.6 Hz, 2H), 3.73 (m, 4H), 3.91 (m, 1H), 4.00 (m, 2H), 4.32 (m, 2H), 4.54 (m, 2H), 4.85 (m, 2H), 5.47 (m, 1H), 7.12 (m, 1H), 7.25 (s, 1H), 7.32-7.38 (m, 1H), 7.41-7.53 (m, 1H), 7.88 (m, 2H), 8.16 (m, 1H), 8.31-8.51 (m, 1H), 9.11 (s, 1H). m/z (ESI): 1012.6 [M+H]⁺.

### Example 126. Synthesis of Compound 159 Salt

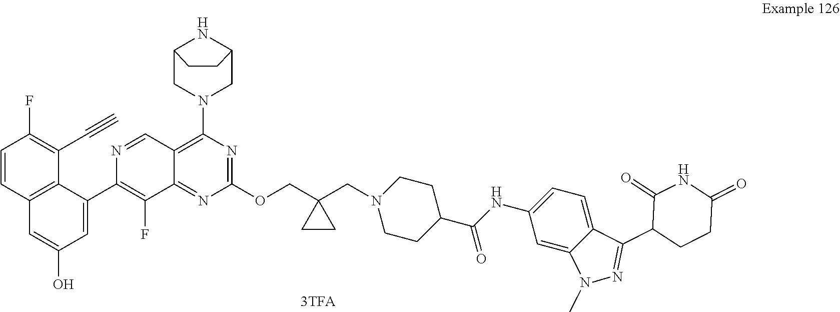 [SMILES: C#Cc1c(F)ccc2cc(O)cc(-c3ncc4c(N5CC6CCC(C5)N6)nc(OCC5(CN6CCC(C(=O)Nc7ccc8c(C9CCC(=O)NC9=O)nn(C)c8c7)CC6)CC5)nc4c3F)c12]

Target compound was synthesized according to the procedure of Example 80. ¹H NMR (500 MHz, Methanol-d4) δ 0.89 (s, 2H), 1.01 (s, 2H), 1.29-1.36 (m, 2H), 2.17 (m, 4H), 2.25-2.29 (m, 4H), 2.33 (m, 1H), 2.47 (m, 1H), 2.69-2.89 (m, 3H), 3.10 (m, 2H), 3.34-3.41 (m, 2H), 3.96 (m, 2H), 3.98 (s, 3H), 4.05 (m, 1H), 4.29 (m, 2H), 4.36 (dd, J=9.3, 5.1 Hz, 1H), 4.47-4.60 (m, 2H), 4.81 (m, 2H), 7.10-7.16 (m, 1H), 7.22 (m, 1H), 7.29 (m, 1H), 7.36 (mz, 1H), 7.67 (m, 1H), 7.81-7.89 (m, 1H), 8.02 (s, 1H), 9.09 (s, 1H). m/z (ESI): 895.5 [M+H]⁺.

### Example 127. Synthesis of Compound 160

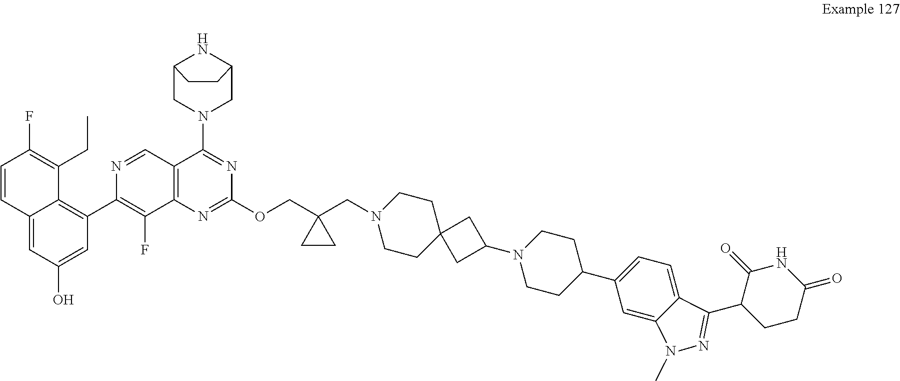 [SMILES: CCc1c(F)ccc2cc(O)cc(-c3ncc4c(N5CC6CCC(C5)N6)nc(OCC5(CN6CCC7(CC6)CC(N6CCC(c8ccc9c(C%10CCC(=O)NC%10=O)nn(C)c9c8)CC6)C7)CC5)nc4c3F)c12]

Target compound was synthesized according to the procedure of Example 80. ¹H NMR (500 MHz, Methanol-d4) δ 0.53 (s, 2H), 0.73 (s, 2H), 0.81 (d, J=5.0 Hz, 3H), 1.56-1.70 (m, 6H), 1.76-2.06 (m, 12H), 2.16-2.24 (m, 1H), 2.26-2.37 (m, 2H), 2.39-2.56 (m, 6H), 2.69-2.82 (m, 4H), 3.07 (d, J=10.0 Hz, 2H), 3.63-3.76 (m, 4H), 4.01 (s, 3H), 4.31-4.47 (m, 3H), 4.55-4.70 (m, 3H), 7.04-7.11 (m, 2H), 7.25 (d, J=10.0 Hz, 1H), 7.30 (d, J=5.0 Hz, 1H), 7.35 (s, 1H), 7.60-7.71 (m, 2H), 9.05 (s, 1H). m/z (ESI): 979.8 [M+H]⁺.

### Example 128. Synthesis of Compound 161

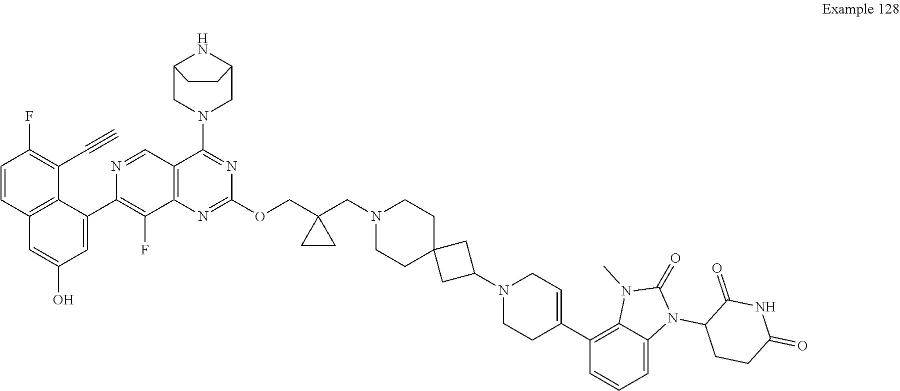 [SMILES: C#Cc1c(F)ccc2cc(O)cc(-c3ncc4c(N5CC6CCC(C5)N6)nc(OCC5(CN6CCC7(CC6)CC(N6CC=C(c8cccc9c8n(C)c(=O)n9C8CCC(=O)NC8=O)CC6)C7)CC5)nc4c3F)c12]

Target compound was synthesized according to the procedure of Example 80. ¹H NMR (500 MHz, Methanol-d4) δ 0.49-0.59 (m, 2H), 0.70-0.77 (m, 2H), 1.62-1.74 (m, 6H), 1.78-1.91 (m, 4H), 2.03-2.10 (m, 2H), 2.13-2.19 (m, 1H), 2.35 (d, J=10.0 Hz, 1H), 2.45-2.52 (m, 4H), 2.59-2.70 (m, 2H), 2.76-2.85 (m, 2H), 2.87-2.95 (m, 2H), 3.21-3.27 (m, 6H), 3.38 (s, 1H), 3.46 (s, 3H), 3.53-3.57 (m, 1H), 3.64-3.75 (m, 4H), 4.33-4.40 (m, 1H), 4.43-4.49 (m, 1H), 4.54-4.60 (m, 1H), 4.61-4.69 (m, 1H), 5.30-5.39 (m, 1H), 5.70 (s, 1H), 6.85-6.91 (m, 1H), 7.01-7.09 (m, 2H), 7.19-7.23 (m, 1H), 7.27-7.36 (m, 2H), 7.85 (dd, J=10.0, 5.0 Hz, 1H), 9.00 (s, 1H). m/z (ESI): 989.7 [M+H]⁺.

### Example 129. Synthesis of Compound 162 Salt

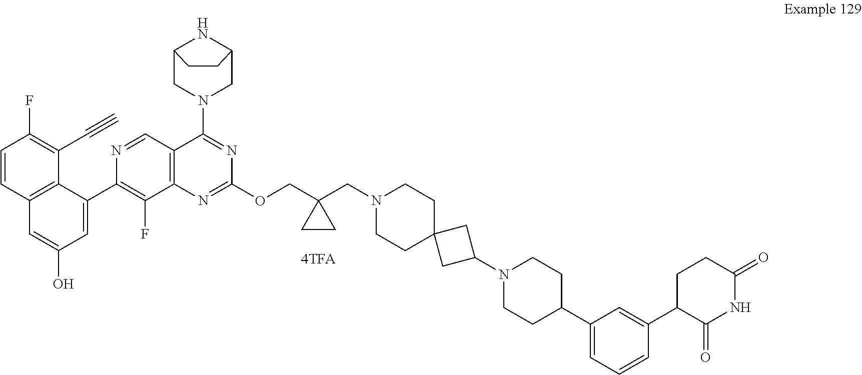 [SMILES: C#Cc1c(F)ccc2cc(O)cc(-c3ncc4c(N5CC6CCC(C5)N6)nc(OCC5(CN6CCC7(CC6)CC(N6CCC(c8cccc(C9CCC(=O)NC9=O)c8)CC6)C7)CC5)nc4c3F)c12]

Target compound was synthesized according to the procedure of Example 80. ¹H NMR (500 MHz, CD₃OD) δ ppm 9.08 (s, 1H), 7.88 (dd, J=8.8, 5.8 Hz, 1H), 7.44-7.30 (m, 3H), 7.26-7.09 (m, 4H), 4.88-4.76 (m, 2H), 4.51 (dt, J=25.0, 12.1 Hz, 2H), 4.29 (d, J=13.7 Hz, 2H), 4.05-3.98 (m, 1H), 3.96-3.83 (m, 3H), 3.79-3.67 (m, 2H), 3.60-3.51 (m, 2H), 3.46-3.37 (m, 2H), 3.27-3.15 (m, 1H), 3.13-2.84 (m, 5H), 2.77-2.48 (m, 3H), 2.32-1.90 (m, 17H), 1.05-0.95 (m, 2H), 0.95-0.86 (m, 2H). m/z (ESI): 921.7 [M+H]⁺.

### Example 130. Synthesis of Compound 163

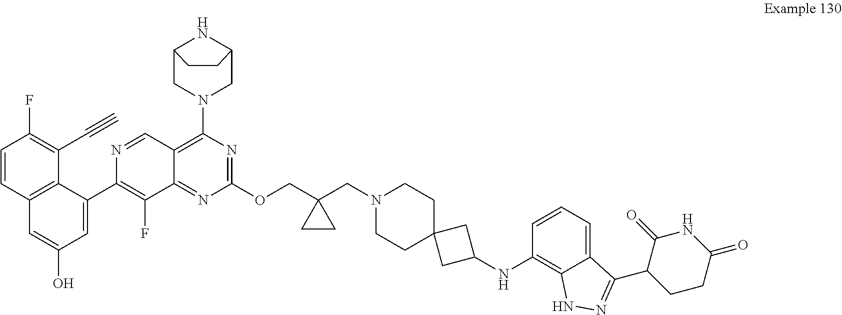 [SMILES: C#Cc1c(F)ccc2cc(O)cc(-c3ncc4c(N5CC6CCC(C5)N6)nc(OCC5(CN6CCC7(CC6)CC(Nc6cccc8c(C9CCC(=O)NC9=O)n[nH]c68)C7)CC5)nc4c3F)c12]

Target compound was synthesized according to the procedure of Example 80. ¹H NMR (500 MHz, Methanol-d4) δ 0.90 (s, 2H), 1.00 (s, 2H), 1.29 (d, J=5.2 Hz, 9H), 1.61 (d, J=7.6 Hz, 2H), 1.81 (d, J=12.9 Hz, 1H), 2.03 (d, J=5.6 Hz, 3H), 2.19 (t, J=7.6 Hz, 2H), 2.38-2.50 (m, 1H), 2.73 (t, J=6.5 Hz, 2H), 3.04 (d, J=31.5 Hz, 1H), 3.37-3.41 (m, 2H), 3.67-3.81 (m, 2H), 3.94 (dd, J=25.3, 14.0 Hz, 2H), 4.10 (q, J=7.2 Hz, 1H), 4.26 (s, 2H), 4.34-4.45 (m, 2H), 4.63 (d, J=12.2 Hz, 1H), 4.82 (d, J=13.8 Hz, 2H), 5.34 (t, J=5.0 Hz, 1H), 6.34 (d, J=7.3 Hz, 1H), 6.97 (t, J=7.7 Hz, 1H), 7.03 (d, J=8.1 Hz, 1H), 7.23 (d, J=8.0 Hz, 2H), 7.35 (dt, J=11.8, 5.7 Hz, 2H), 7.79-7.90 (m, 1H), 9.09 (s, 1H). m/z (ESI): 892.40 [M+H]⁺.

### Example 131. Synthesis of Compound 164

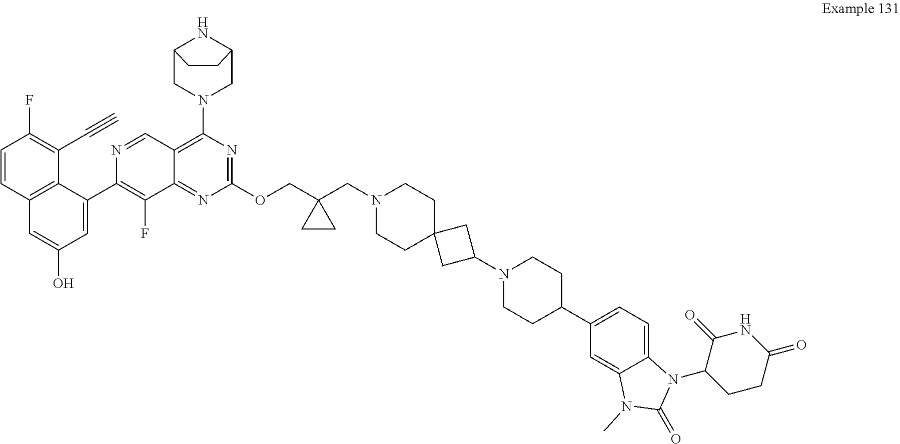 [SMILES: C#Cc1c(F)ccc2cc(O)cc(-c3ncc4c(N5CC6CCC(C5)N6)nc(OCC5(CN6CCC7(CC6)CC(N6CCC(c8ccc9c(c8)n(C)c(=O)n9C8CCC(=O)NC8=O)CC6)C7)CC5)nc4c3F)c12]

Target compound was synthesized according to the procedure of Example 80. ¹H NMR (500 MHz, Methanol-d4) δ 0.90 (s, 2H), 0.96-1.03 (m, 2H), 1.29 (d, J=4.2 Hz, 6H), 1.60 (t, J=7.3 Hz, 2H), 2.02-2.06 (m, 5H), 2.17 (d, J=9.4 Hz, 4H), 2.27 (d, J=8.8 Hz, 2H), 2.54 (s, 1H), 2.80 (dd, J=15.1, 4.2 Hz, 2H), 2.90-2.95 (m, 3H), 2.99-3.13 (m, 2H), 3.35 (s, 1H), 3.43 (s, 3H), 3.52-3.61 (m, 2H), 3.73 (q, J=9.2, 8.5 Hz, 2H), 3.85 (d, J=12.9 Hz, 1H), 3.93 (d, J=14.2 Hz, 1H), 4.01 (d, J=13.9 Hz, 1H), 4.29 (d, J=13.7 Hz, 2H), 4.46 (t, J=10.9 Hz, 1H), 4.55 (t, J=10.7 Hz, 1H), 5.29-5.38 (m, 3H), 7.03 (d, J=8.3 Hz, 1H), 7.08 (d, J=7.1 Hz, 2H), 7.22 (d, J=2.5 Hz, 1H), 7.32-7.38 (m, 2H), 7.87 (dd, J=9.2, 5.7 Hz, 1H), 9.07 (s, 1H). m/z (ESI): 990.47 [M+H]⁺.

### Example 132. Synthesis of Compound 165

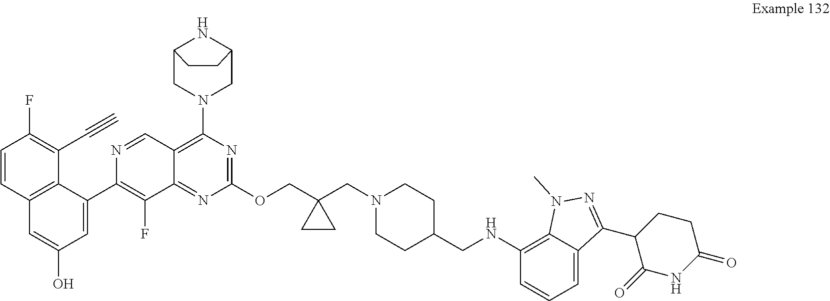 [SMILES: C#Cc1c(F)ccc2cc(O)cc(-c3ncc4c(N5CC6CCC(C5)N6)nc(OCC5(CN6CCC(CNc7cccc8c(C9CCC(=O)NC9=O)nn(C)c78)CC6)CC5)nc4c3F)c12]

Target compound was synthesized according to the procedure of Example 80. ¹H NMR (500 MHz, Methanol-d4) δ 0.87-0.92 (m, 2H), 0.99 (s, 2H), 1.28-1.36 (m, 6H), 1.71 (s, 2H), 2.03 (s, 1H), 2.11 (s, 3H), 2.28 (dd, J=12.9, 6.1 Hz, 1H), 2.42 (d, J=8.4 Hz, 1H), 2.75 (dt, J=9.2, 5.7 Hz, 2H), 3.00 (q, J=11.6 Hz, 2H), 3.10 (d, J=6.6 Hz, 2H), 3.18 (dd, J=14.6, 7.9 Hz, 1H), 3.41 (d, J=15.2 Hz, 2H), 3.84 (s, 3H), 3.99 (d, J=13.6 Hz, 2H), 4.16 (s, 2H), 4.26 (dd, J=9.7, 4.8 Hz, 2H), 4.46 (d, J=11.6 Hz, 1H), 4.58 (d, J=12.4 Hz, 1H), 4.77 (d, J=14.2 Hz, 2H), 6.34 (s, 1H), 6.58 (d, J=8.9 Hz, 1H), 7.22 (d, J=2.4 Hz, 1H), 7.26 (d, J=8.8 Hz, 1H), 7.34 (t, J=3.0 Hz, 1H), 7.37 (dd, J=8.7, 4.3 Hz, 1H), 7.79 (dt, J=9.3, 5.0 Hz, 1H), 9.07 (s, 1H). m/z (ESI): 880.40 [M+H]⁺.

### Example 133. Synthesis of Compound 166 Salt

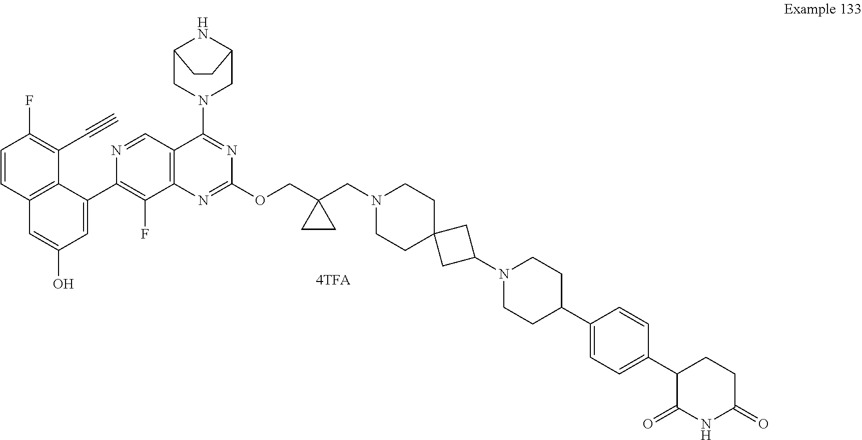 [SMILES: C#Cc1c(F)ccc2cc(O)cc(-c3ncc4c(N5CC6CCC(C5)N6)nc(OCC5(CN6CCC7(CC6)CC(N6CCC(c8ccc(C9CCC(=O)NC9=O)cc8)CC6)C7)CC5)nc4c3F)c12]

Target compound was synthesized according to the procedure of Example 80. ¹H NMR (500 MHz, CD₃OD) δ ppm 9.08 (s, 1H), 8.13 (t, J=7.6 Hz, 1H), 8.00 (d, J=8.1 Hz, 1H), 7.88 (dd, J=8.7, 5.9 Hz, 1H), 7.45 (dd, J=27.3, 8.0 Hz, 1H), 7.39-7.18 (m, 4H), 4.80 (d, J=15.2 Hz, 2H), 4.48 (tt, J=17.1, 10.2 Hz, 2H), 4.29 (d, J=13.5 Hz, 2H), 4.20-3.49 (m, 10H), 3.48-3.36 (m, 2H), 3.26-2.89 (m, 6H), 2.76-2.40 (m, 3H), 2.40-1.90 (m, 16H), 1.05-0.95 (m, 2H), 0.92-0.88 (m, 2H). m/z (ESI): 921.6 [M+H]⁺.

### Example 134. Synthesis of Compound 167 Salt

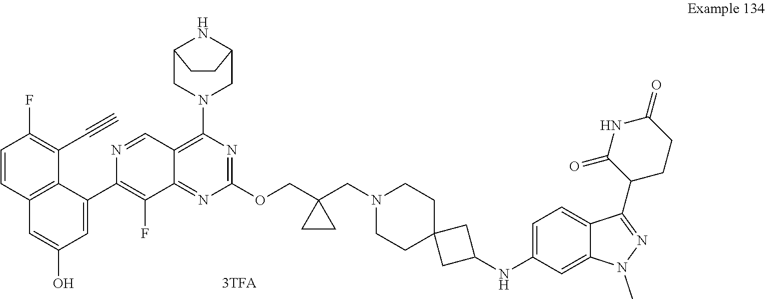 [SMILES: C#Cc1c(F)ccc2cc(O)cc(-c3ncc4c(N5CC6CCC(C5)N6)nc(OCC5(CN6CCC7(CC6)CC(Nc6ccc8c(C9CCC(=O)NC9=O)nn(C)c8c6)C7)CC5)nc4c3F)c12]

Target compound was synthesized according to the procedure of Example 80. ¹H NMR (500 MHz, Methanol-d4) δ 0.89 (m, 2H), 1.00 (m, 2H), 1.80-2.07 (m, 5H), 2.13 (m, 5H), 2.32 (m, 2H), 2.43 (m, 1H), 2.57 (m, 1H), 2.69-2.83 (m, 2H), 2.98 (m, 1H), 3.09 (m, 1H), 3.16-3.27 (m, 1H), 3.35-3.46 (m, 2H), 3.75 (m, 1H), 3.80-3.97 (m, 5H), 4.02 (m, 2H), 4.27 (s, 3H), 4.40-4.66 (m, 2H), 4.81 (m, 2H), 6.41 (d, J=22.6 Hz, 1H), 6.65 (d, J=8.6 Hz, 1H), 7.20-7.40 (m, 3H), 7.48 (m, 1H), 7.74-7.85 (m, 1H), 9.09 (s, 1H). m/z (ESI): 907.7 [M+H]⁺.

### Example 135. Synthesis of Compound 168

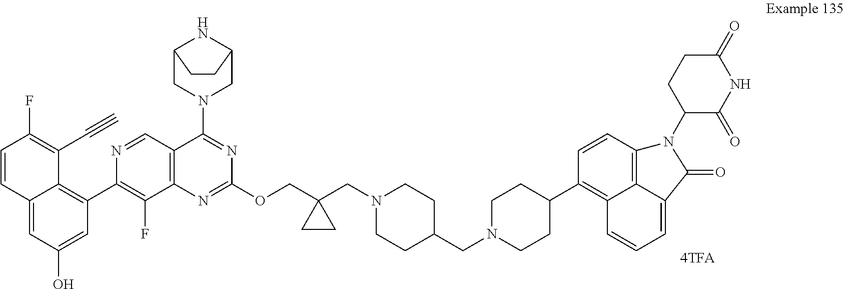 [SMILES: C#Cc1c(F)ccc2cc(O)cc(-c3ncc4c(N5CC6CCC(C5)N6)nc(OCC5(CN6CCC(CN7CCC(c8ccc9c%10c(cccc8%10)C(=O)N9C8CCC(=O)NC8=O)CC7)CC6)CC5)nc4c3F)c12]

Target compound was synthesized according to the procedure of Example 80. ¹H NMR (500 MHz, CD₃OD) δ ppm 9.08 (s, 1H), 8.42 (d, J=8.1 Hz, 1H), 8.12 (d, J=6.8 Hz, 1H), 7.91-7.80 (m, 2H), 7.40 (d, J=7.2 Hz, 1H), 7.37-7.28 (m, 2H), 7.23 (s, 1H), 7.08 (d, J=7.3 Hz, 1H), 5.48-5.39 (m, 1H), 4.79 (d, J=14.6 Hz, 1H), 4.62 (d, J=11.8 Hz, 1H), 4.44 (d, J=11.8 Hz, 1H), 4.31 (d, J=16.4 Hz, 2H), 4.05 (t, J=13.2 Hz, 2H), 3.93 (t, J=12.9 Hz, 2H), 3.85-3.40 (m, 6H), 3.28-2.93 (m, 6H), 2.85 (dd, J=19.6, 9.7 Hz, 2H), 2.39-1.99 (m, 11H), 1.83 (d, J=12.2 Hz, 2H), 1.36 (dd, J=21.3, 15.2 Hz, 3H), 1.01 (d, J=9.4 Hz, 2H), 0.96-0.86 (m, 2H). m/z (ESI): 986.7 [M+H]⁺.

### Example 136. Synthesis of Compound 169 Salt

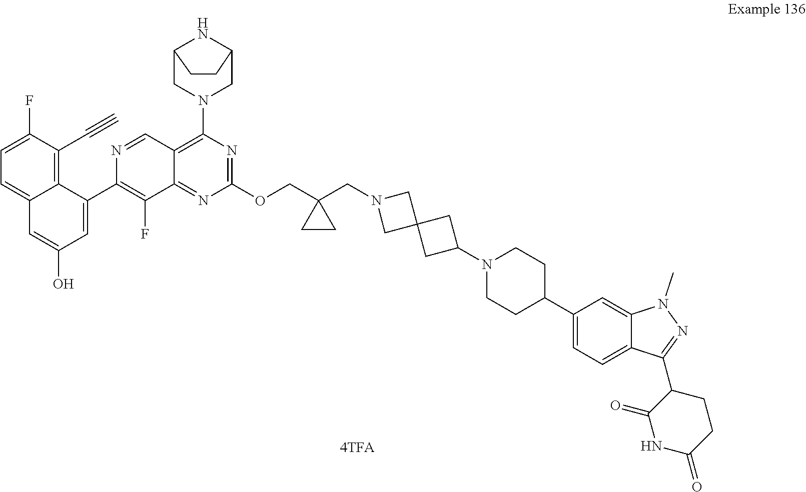 [SMILES: C#Cc1c(F)ccc2cc(O)cc(-c3ncc4c(N5CC6CCC(C5)N6)nc(OCC5(CN6CC7(CC(N8CCC(c9ccc%10c(C%11CCC(=O)NC%11=O)nn(C)c%10c9)CC8)C7)C6)CC5)nc4c3F)c12]

Target compound was synthesized according to the procedure of Example 80. ¹H NMR (500 MHz, Methanol-d4) δ 0.89 (m, 4H), 2.00-2.21 (m, 8H), 2.31 (m, 1H), 2.46 (m, 1H), 2.58-2.82 (m, 5H), 2.86-2.95 (m, 3H), 3.00-3.05 (m, 1H), 3.34-3.40 (m, 2H), 3.44-3.58 (m, 3H), 3.65 (m, 1H), 3.94 (m, 1H), 4.01 (s, 3H), 4.24-4.40 (m, 6H), 4.45-4.63 (m, 2H), 4.78-4.90 (m, 4H), 7.07 (d, J=8.5 Hz, 1H), 7.24 (s, 1H), 7.29-7.43 (m, 3H), 7.69 (d, J=8.4 Hz, 1H), 7.88 (m, 1H), 9.10 (s, 1H). m/z (ESI): 947.6 [M+H]⁺.

### Example 137. Synthesis of Compound 170 Salt

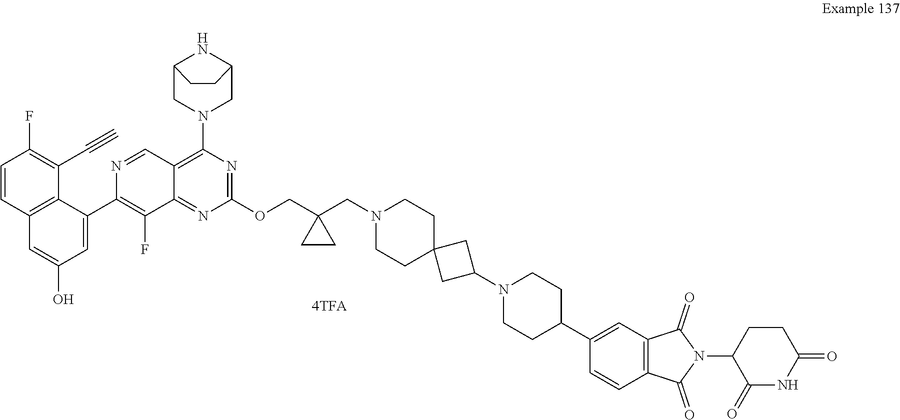 [SMILES: C#Cc1c(F)ccc2cc(O)cc(-c3ncc4c(N5CC6CCC(C5)N6)nc(OCC5(CN6CCC7(CC6)CC(N6CCC(c8ccc9c(c8)C(=O)N(C8CCC(=O)NC8=O)C9=O)CC6)C7)CC5)nc4c3F)c12]

Target compound was synthesized according to the procedure of Example 80. ¹H NMR (500 MHz, Methanol-d4) δ 0.89 (m, 2H), 1.02 (m, 2H), 1.86-2.33 (m, 16H), 2.54 (m, 1H), 2.67-2.81 (m, 2H), 2.84-3.27 (m, 7H), 3.36-3.50 (m, 2H), 3.60 (m, 2H), 3.70-3.79 (m, 2H), 3.90 (m, 2H), 4.03 (m, 1H), 4.29 (d, J=15.3 Hz, 2H), 4.45 (t, J=11.3 Hz, 1H), 4.57 (t, J=10.8 Hz, 1H), 4.75-4.90 (m, 2H), 5.15 (dd, J=12.7, 5.4 Hz, 1H), 7.23 (d, J=2.5 Hz, 1H), 7.30-7.39 (m, 2H), 7.76 (d, J=7.7 Hz, 1H), 7.81 (s, 1H), 7.88 (d, J=7.7 Hz, 2H), 9.10 (s, 1H). m/z (ESI): 990.5 [M+H]⁺.

### Example 138. Synthesis of Compound 171

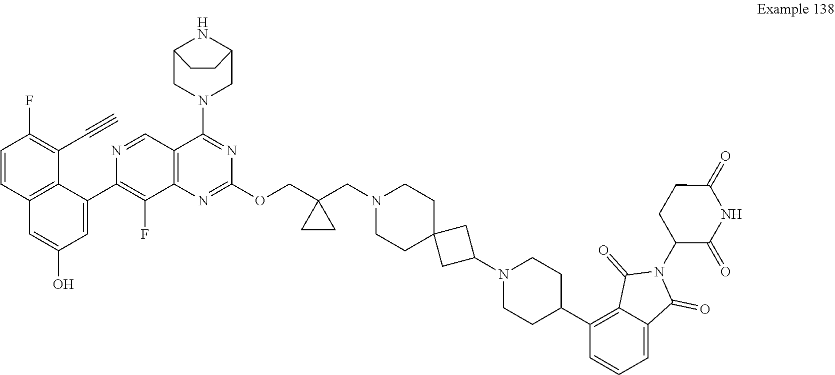 [SMILES: C#Cc1c(F)ccc2cc(O)cc(-c3ncc4c(N5CC6CCC(C5)N6)nc(OCC5(CN6CCC7(CC6)CC(N6CCC(c8cccc9c8C(=O)N(C8CCC(=O)NC8=O)C9=O)CC6)C7)CC5)nc4c3F)c12]

Target compound was synthesized according to the procedure of Example 80. ¹H NMR (500 MHz, Methanol-d4) δ 0.54 (s, 2H), 0.74 (s, 2H), 1.57-1.62 (m, 2H), 1.63-1.72 (m, 4H), 1.79-1.93 (m, 8H), 1.93-2.01 (m, 2H), 2.02-2.09 (m, 2H), 2.13-2.20 (m, 1H), 2.40-2.54 (m, 4H), 2.71-2.83 (m, 3H), 2.85-2.95 (m, 1H), 3.07 (d, J=10.0 Hz, 2H), 3.40 (s, 1H), 3.66-3.84 (m, 5H), 4.34-4.41 (m, 1H), 4.44-4.50 (m, 1H), 4.60 (d, J=15.0 Hz, 1H), 4.67 (d, J=15.0 Hz, 1H), 5.16 (dd, J=15.0, 10.0 Hz, 1H), 7.23 (s, 1H), 7.30-7.39 (m, 2H), 7.74-7.83 (m, 3H), 7.85-7.92 (m, 1H), 9.02 (s, 1H). m/z (ESI): 990.7 [M+H]⁺.

### Example 139. Synthesis of Compound 1a

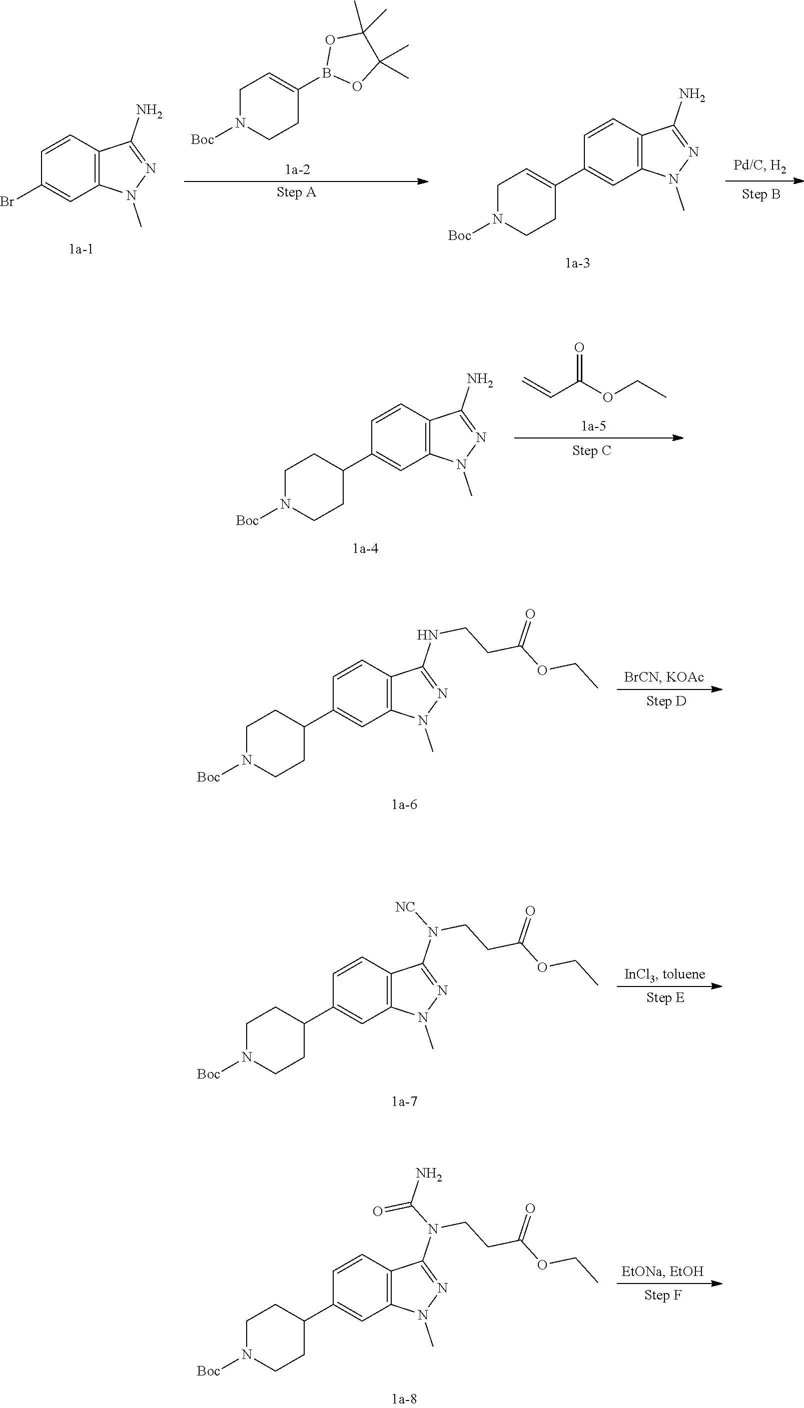

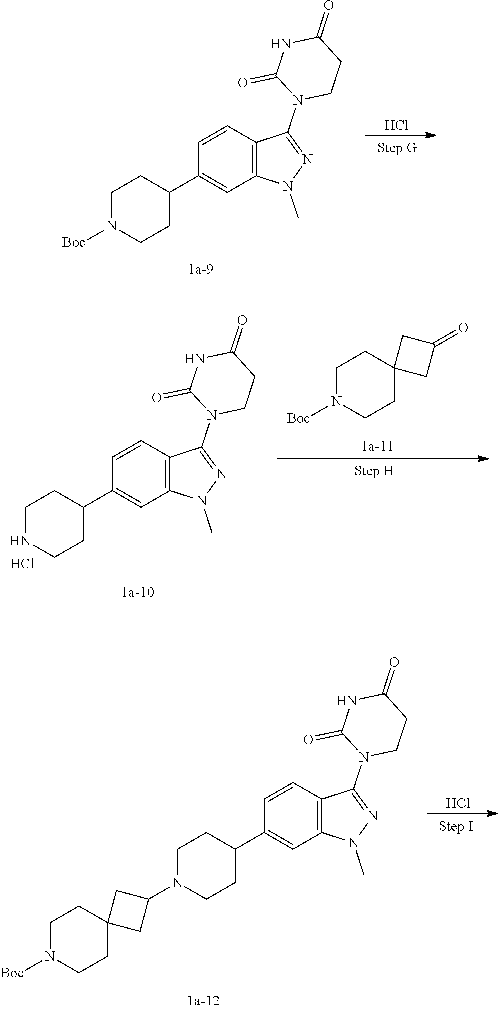

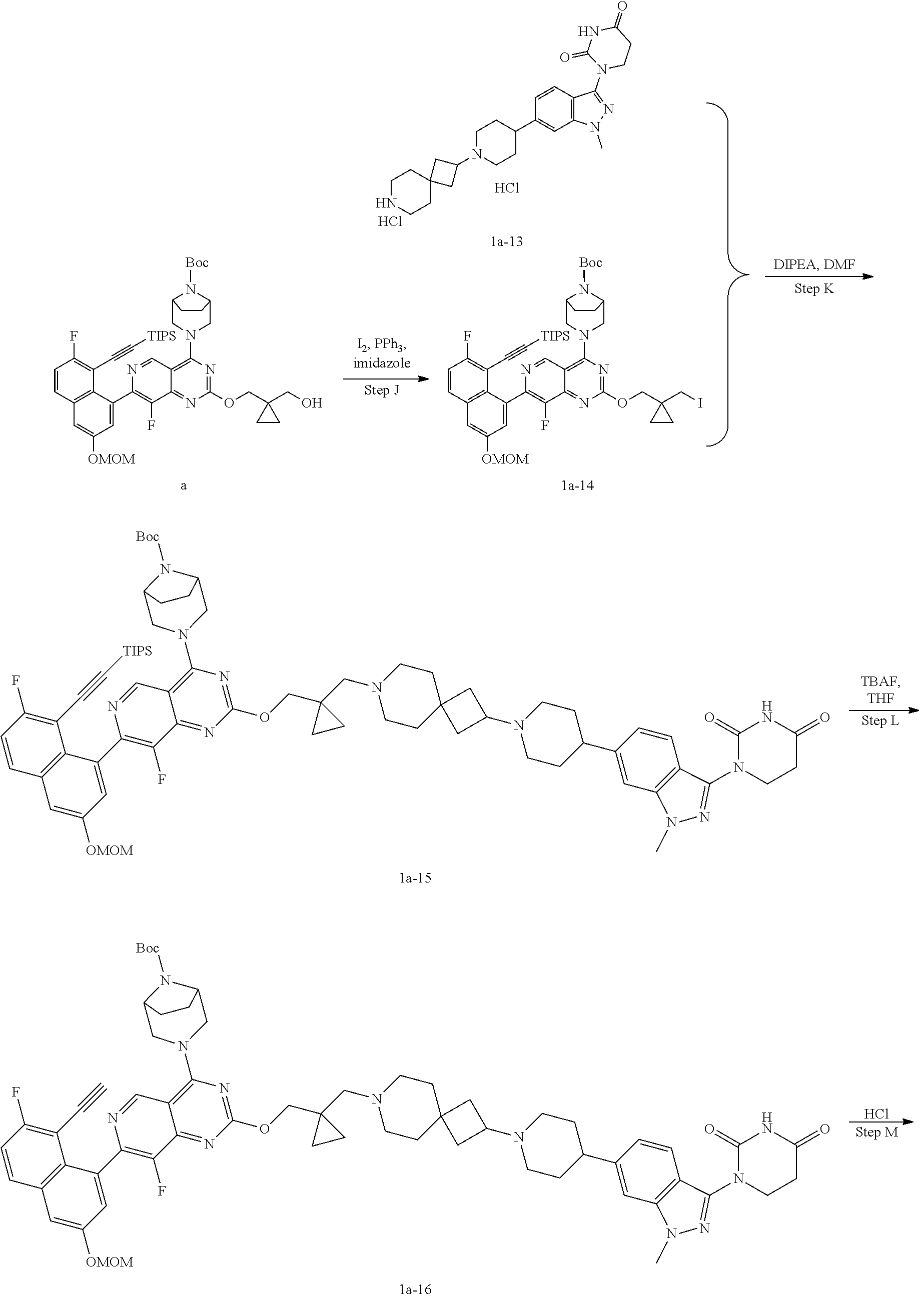

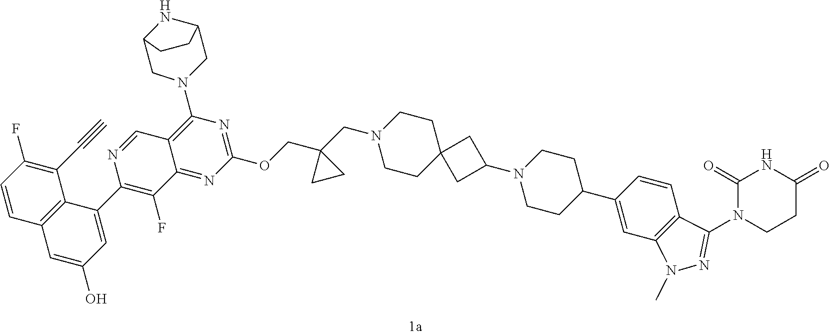 [SMILES: C#Cc1c(F)ccc2cc(O)cc(-c3ncc4c(N5CC6CCC(C5)N6)nc(OCC5(CN6CCC7(CC6)CC(N6CCC(c8ccc9c(N%10CCC(=O)NC%10=O)nn(C)c9c8)CC6)C7)CC5)nc4c3F)c12]

Step A: Compound 1a-2 (2.05 g, 6.64 mmol, 1.5 eq) was added to a solution of compound 1a-1 (1 g, 4.42 mmol, 1 eq) in dioxane (10 mL) and water (1 mL), followed by addition of K₃PO₄ (2.82 g, 13.27 mmol, 3 eq) and cataCXium A Pd G3 (643.40 mg, 884.67 μmol, 0.2 eq). The mixture was warmed to 100° C. under the nitrogen atmosphere and stirred for 3 h, then cooled to 25° C. The mixture was quenched with water and extracted with DCM. The combined organic layer was washed with water and brine, then dried over Na₂SO₄ and filtered. The filtrate was concentrated under reduced pressure and the residue was purified by column chromatography (EA/DCM=30%-70%) to afford compound 1a-3 (1.3 g, yield 89.490%).

Step B: 100% Pd/C (400 mg) was added to a solution of compound 1a-3 (1.1 g, 3.35 mmol, 1 eq) in THF (15 mL). The mixture was stirred at 40° C. under hydrogen atmosphere for 2 h, then cooled to 25° C. and filtered. The filtrate was concentrated under reduced pressure to afford compound 1a-4 (1 g, yield 90.35%).

Step C: Mixture of compound 1a-4 (2 g, 6.05 mmol, 1 eq) and compound 1a-5 (4.24 g, 42.37 mmol, 7 eq) was added to another mixture of DBU in LAC (1/1, 1.61 g, 6.66 mmol, 1.1 eq). The reaction mixture was warmed to 80° C. and stirred for 2 days, then cooled to 25° C. The reaction was quenched with water, and extracted with EA. The combined organic layer was washed with water and brine, then dried over Na₂SO₄ and filtered. The filtrate was concentrated under reduced pressure and the residue was purified by column chromatography (EA/PE=0%-50%) to afford compound 1a-6 (1.1 g, yield 42.21%).

Step D: Cyanogen bromide (842.75 mg, 7.66 mmol, 3 eq) was added to a solution of compound 1a-6 (1.1 g, 2.55 mmol, 1 eq) in EtOH (15 mL), followed by addition of AcOK (752.22 mg, 7.66 mmol, 3 eq). The mixture was stirred at 80° C. for 16 h, then cooled to 25° C. The reaction was quenched with water, and extracted with EA. The combined organic layer was washed with water and brine, then dried over Na₂SO₄ and filtered. The filtrate was concentrated under reduced pressure and the residue was purified by column chromatography (MeOH/DCM=0%-10%) to afford compound 1a-7 (1.0 g, yield 85.92%).

Step E: Compound g-4 (194.49 mg, 3.29 mmol, 1.5 eq) was added to a solution of compound 1a-7 (1 g, 2.20 mmol, 1 eq) in toluene (20 mL), followed by addition of InCl₃ (139.01 mg, 658.55 μmol, 0.3 eq). The mixture was stirred at 110° C. for 2 h, then cooled to 25° C. The mixture was concentrated under reduced pressure and the residue was purified by column chromatography (MeOH/DCM=0%-5%) to afford compound 1a-8 (870 mg, yield 83.69%).

Step F: EtONa (286.5 mg, 5.51 mmol, 3 eq) was added to a solution of compound 1a-8 (870 mg, 1.84 mmol, 1 eq) in EtOH (2 mL) under nitrogen atmosphere. The mixture was stirred at 25° C. for 3 h, then quenched with water and extracted with EA. The combined organic layer was washed with water and brine, then dried over Na₂SO₄ and filtered. The filtrate was concentrated under reduced pressure and the residue was purified by column chromatography (MeOH/DCM=0%-3%) to afford compound 1a-9 (650 mg, yield 82.76%).

Step G: 4M HCl in dioxane (7.6 mL) was added to a solution of compound 1a-9 (650 mg, 1.52 mmol, 1 eq) in DCM (10 mL). The mixture was stirred at 25° C. for 2 h, then concentrated under reduced pressure to afford compound 1a-10 (490 mg, yield 98.44%).

Step H: AcOH (18.4 mg, 305.46 μmol, 1 eq) was added to a solution of compound 1a-10 (111.14 mg, 305.46 μmol, 1 eq) in DMF (2 mL). The mixture was stirred for 10 min, then followed by addition of compound 1a-11 (73.10 mg, 305.46 μmol, 1 eq) and NaBH₃CN (19.19 mg, 305.46 μmol, 1 eq). The mixture was stirred at 25° C. for 2 h, followed by addition of NaBH₃CN (19.19 mg, 305.46 μmol, 1 eq), then stirred for 16 h. The pH of the mixture was adjusted to 9-10 with NaHCO₃ aqueous solution. The mixture was extracted with EA, and the combined organic layer was washed with water and brine, then dried over Na₂SO₄ and filtered. The filtrate was concentrated under reduced pressure and the residue was purified by column chromatography (MeOH/DCM=0%-7%) to afford compound 1a-12 (130 mg, yield 77.28%).

Step I: 2M HCl in EA (1.2 mL) was added to a solution of compound 1a-12 (130 mg, 236.07 μmol, 1 eq) in DCM (2 mL). The mixture was stirred at 25° C. for 2 h, then concentrated under reduced pressure to afford compound 1a-13 (100 mg, yield 80.92%).

Step J: Imidazole (806.57 mg, 11.85 mmol, 4.0 eq) was added to a solution of intermediate a (2.5 g, 2.96 mmol, 1 eq) in DCM (50 mL), followed by addition of PPh₃ (1.94 g, 7.40 mmol, 2.5 eq) and 12 (1.88 g, 7.40 mmol, 2.5 eq). The mixture was stirred at 25° C. for 16 h, then quenched with water and extracted with EA. The combined organic layer was washed with water and brine, then dried over Na₂SO₄ and filtered. The filtrate was concentrated under reduced pressure and the residue was purified by column chromatography (EA/PE=0%-37%) to afford compound 1a-14 (2.3 g, yield 81.4%).

Step K: DIPEA (86.05 mg, 665.82 μmol, 3 eq) was added to a solution of compound 1a-13 (100 mg, 221.94 μmol, 1 eq) in DMF (2 mL). The mixture was stirred at 25° C. for 10 min, followed by addition of compound 1a-14 (211.72 mg, 221.94 μmol, 1 eq). The mixture was stirred at 45° C. for 16 h, then cooled to 25° C. and quenched with water. The mixture was extracted with EA, and the combined organic layer was washed with water and brine, then dried over Na₂SO₄ and filtered. The filtrate was concentrated under reduced pressure and the residue was purified by column chromatography (MeOH/DCM=0%-7%) to afford compound 1a-15 (100 mg, yield 35.29%).

Step L: 1M TBAF in THF (0.5 mL) was added to a solution of compound 1a-15 (20 mg, 94.00 μmol, 1 eq) in THF (2 mL). The mixture was stirred at 25° C. for 2 h, then quenched with water. The mixture was extracted with EA, and the combined organic layer was washed with water and brine, then dried over Na₂SO₄ and filtered. The filtrate was concentrated under reduced pressure to afford compound 1a-16 (80 mg, yield 75.97%).

Step M: 2M HCl in EA (0.5 mL) was added to a solution of compound 1a-16 (80.00 mg, 71.41 μmol, 1 eq) in DCM (2 mL). The mixture was stirred at 25° C. for 30 min, then concentrated under reduced pressure. The residue was purified by pre-HPLC (0.1% TFA in water/acetonitrile) to afford compound 1a (9.8 mg, yield 18.81%). ¹H NMR (400 MHz, CD₃OD) 0.88-0.96 (m, 2H). 0.9-1.09 (m, 2H), 1.32 (s, 2H), 1.36 (d, J=17.8 Hz, 1H), 2.00-2.37 (m, 16H), 2.59 (q, J=13.2, 8.8 Hz, 1H), 2.90 (t, J=6.7 Hz, 2H), 2.95-3.17 (m, 5H), 3.26 (dd, J=18.2, 13.5 Hz, 2H), 3.42 (d, J=15.7 Hz, 1H), 3.48 (s, 1H), 3.56-3.66 (m, 2H), 3.78 (d, J=10.2 Hz, 2H), 3.92 (dd, J=26.4, 13.6 Hz, 2H), 4.03 (s, 1H), 4.05 (s, 3H), 4.08 (d, J=6.8 Hz, 2H), 4.32 (d, J=11.4 Hz, 2H), 4.44-4.54 (m, 1H), 4.55-4.64 (m, 1H), 4.78-4.89 (m, 3H), 7.12 (d, J=8.5 Hz, 1H), 7.26 (d, J=2.5 Hz, 1H), 7.34-7.44 (m, 3H), 7.70 (d, J=8.6 Hz, 1H), 7.90 (dd, J=9.1, 5.7 Hz, 1H), 9.12 (s, 1H). m/z (ESI): 977.40 [M+H]⁺.

### Example 140. Synthesis of Compound 2a

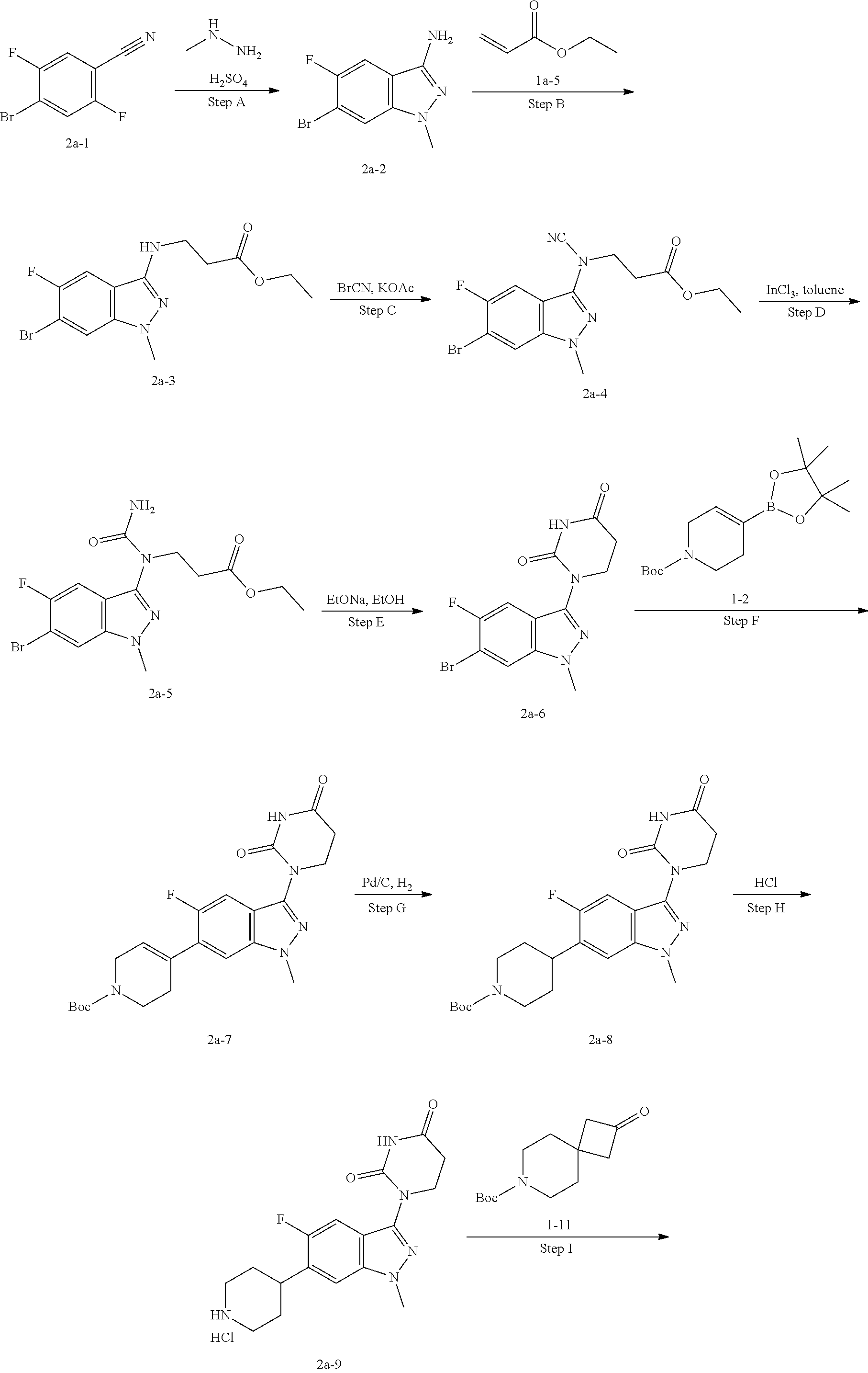

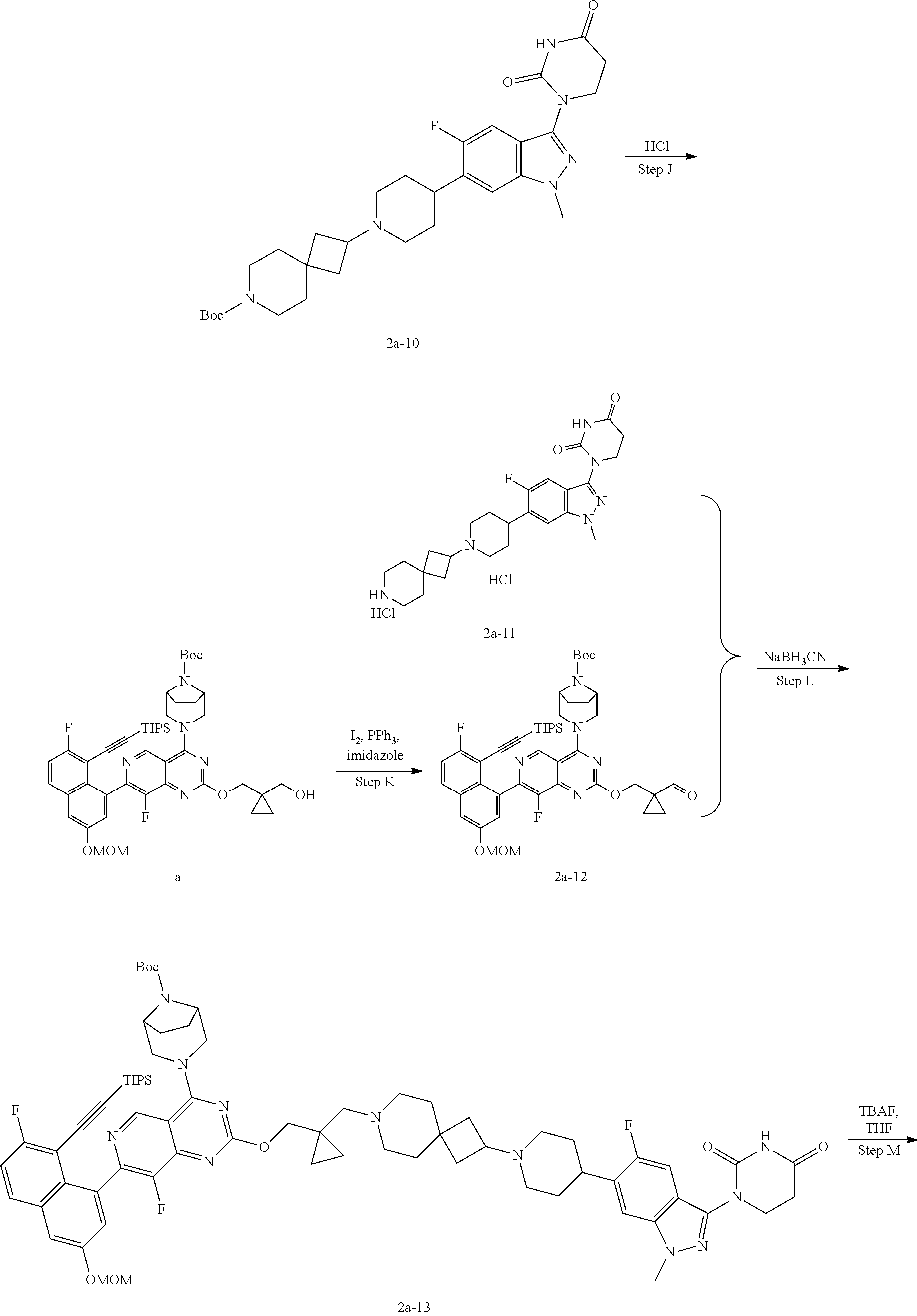

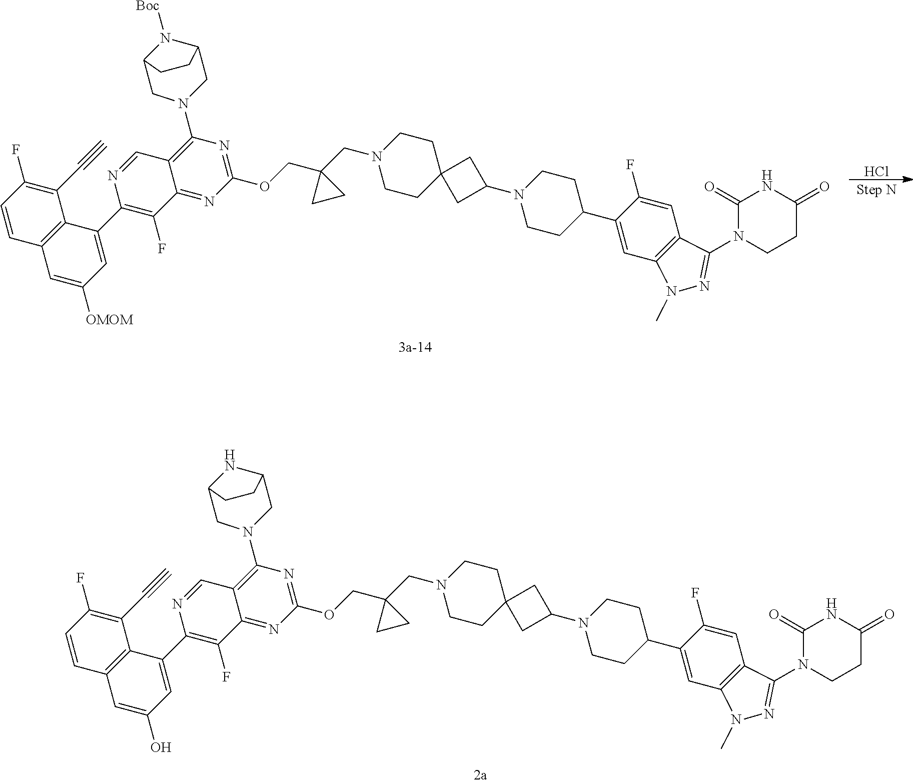

Step A: Methylhydrazine sulfate (28.80 g, 224.77 mmol, 2 eq) was added to a solution of compound 2a-1 (24.5 g, 112.39 mmol, 1 eq) in EtOH (300 mL), followed by addition of K₂CO₃ (34.17 g, 247.25 mmol, 2.2 eq). The mixture was stirred at 80° C. for 18 h, then cooled to 25° C. and quenched with water. The mixture was extracted with EA, and the combined organic layer was washed with water and brine, then dried over Na₂SO₄ and filtered. The filtrate was concentrated under reduced pressure and the residue was purified by column chromatography (EA/PE=0%-35%) to afford compound 2a-2 (17 g, yield 61.98%). ¹H NMR (400 MHz, CD₃OD) δ 7.68 (d, J=5.4 Hz, 1H), 7.51 (d, J=8.6 Hz, 1H), 3.79 (s, 3H).

Step B: Mixture of compound 2a-2 (5 g, 20.49 mmol, 1 eq) and compound 1a-5 (14.36 g, 143.41 mmol, 7 eq) was added to another mixture of DBU in LAC (5.45 g, 22.54 mmol, 1.1 eq). The reaction mixture was warmed to 90° C. and stirred for 3 days, then cooled to 25° C. The reaction was quenched with water, and extracted with EA. The combined organic layer was washed with water and brine, then dried over Na₂SO₄ and filtered. The filtrate was concentrated under reduced pressure and the residue was purified by column chromatography (EA/PE=0%-25%) to afford compound 2a-3 (3.86 g, yield 54.74%).

Step C: Cyanogen bromide (4.40 g, 41.55 mmol, 5 eq) was added to a solution of compound 2a-3 (2.86 g, 8.31 mmol, 1 eq) in EtOH (30 mL), followed by addition of AcOK (4.09 g, 49.86 mmol, 6 eq). The mixture was stirred at 85° C. for 16 h, then cooled to 25° C. The reaction was quenched with water, and extracted with EA. The combined organic layer was washed with water and brine, then dried over Na₂SO₄ and filtered. The filtrate was concentrated under reduced pressure and the residue was purified by column chromatography (PE/DCM=0%-20%) to afford compound 2a-4 (1.5 g, yield 48.89%). ¹H NMR (400 MHz, Chloroform-d) δ 7.80 (d, J=8.4 Hz, 1H), 7.56 (d, J=5.3 Hz, 1H), 4.23 (q, J=7.2 Hz, 2H), 4.14 (t, J=6.9 Hz, 2H), 3.94 (s, 3H), 2.95 (t, J=6.8 Hz, 2H), 1.31 (t, J=7.1 Hz, 3H).

Step D: Compound g-4 (959.95 mg, 16.25 mmol, 3 eq) was added to a solution of compound 2a-4 (2 g, 5.42 mmol, 1 eq) in toluene (30 mL), followed by addition of InCl₃ (119.72 mg, 541.73 μmol, 0.1 eq). The mixture was stirred at 110° C. for 1 h, then cooled to 25° C. The mixture was concentrated under reduced pressure and the residue was purified by column chromatography (MeOH/DCM=0%-2%) to afford compound 2a-5 (2 g, yield 95.35%). ¹H NMR (400 MHz, Chloroform-d) δ 7.67 (d, J=5.1 Hz, 1H), 7.41 (d, J=8.2 Hz, 1H), 4.99 (s, 2H), 4.15 (t, J=7.1 Hz, 2H), 4.11-4.05 (m, 2H), 4.03 (s, 3H), 2.72 (t, J=7.1 Hz, 2H), 1.22 (t, J=7.2 Hz, 3H).

Step E: EtONa (537.19 mg, 10.33 mmol, 2 eq) was added to a solution of compound 2a-5 (2 g, 5.17 mmol, 1 eq) in EtOH (20 mL) under nitrogen atmosphere. The mixture was stirred at 25° C. for 3 h, then quenched with water. The pH was adjusted to 2a-3 with 2N HCl aqueous solution. The precipitate was collected by filter and dried to afford compound 2a-6 (1.4 g, yield 79.45%).

Step F: Compound 1-2 (1.90 g, 6.16 mmol, 1.5 eq) was added to a solution of compound 2a-6 (1.4 g, 4.10 mmol, 1 eq) in dioxane (16 mL) and water (4 mL), followed by addition of K₃PO₄ (2.61 g, 12.31 mmol, 3 eq) and cataCXium A Pd G3 (149.38 mg, 205.20 μmol, 0.05 eq). The mixture was warmed to 90° C. under the nitrogen atmosphere and stirred for 18 h, then cooled to 25° C. The reaction mixture was quenched with water and extracted with DCM. The combined organic layer was washed with water and brine, then dried over Na₂SO₄ and filtered. The filtrate was concentrated under reduced pressure and the residue was purified by column chromatography (MeOH/DCM=0%-2%) to afford compound 2a-7 (1.7 g, yield 93.41%). ¹H NMR (400 MHz, Chloroform-d) δ 7.88 (s, 1H), 7.41 (d, J=10.7 Hz, 1H), 7.22 (d, J=5.7 Hz, 1H), 6.02 (s, 1H), 4.05 (s, 3H), 3.71 (t, J=5.4 Hz, 2H), 2.95 (t, J=6.7 Hz, 2H), 2.59 (s, 2H), 1.77 (s, 4H), 1.56 (s, 9H).

Step G: 10% Pd/C (500 mg) was added to a solution of compound 2a-7 (1.7 g, 3.83 mmol, 1 eq) in MeOH (50 mL). The mixture was stirred at 50° C. under hydrogen atmosphere for 24 h, then cooled to 25° C. and filtered. The filtrate was concentrated under reduced pressure and the residue was purified by column chromatography (MeOH/DCM=0%-1%) to afford compound 2a-8 (380 mg, yield 22.25%).

Step H: 2M HCl in EA (0.5 mL) was added to a solution of compound 2a-8 (150 mg, 336.71 μmol, 1 eq) in DCM (0.5 mL). The mixture was stirred at 25° C. for 15 min and concentrated under reduced pressure to afford compound 2a-9 (128 mg, yield 99.56%).

Step I: Et₃N (166.96 mg, 1.65 mmol, 5 eq) was added to a solution of compound 2a-9 (126 mg, 329.99 μmol, 1 eq) in THF (1 mL) and MeOH (1 mL), followed by addition of AcOH (19.8 mg, 330 μmol, 1 eq). The mixture was stirred at 25° C. for 10 min, followed by addition of compound 1a-11 (94.76 mg, 395.99 μmol, 1.2 eq) and NaBH₃CN (61.38 mg, 989.96 μmol, 3 eq). The reaction mixture was stirred at 25° C. for 4 h, then warmed to 50° C. and stirred for 16 h, then cooled to 25° C. The reaction mixture was quenched with water, and extracted with EA. The combined organic layer was washed with water and brine, then dried over Na₂SO₄ and filtered. The filtrate was concentrated under reduced pressure and the residue was purified by column chromatography (MeOH/DCM=0%-10%) to afford compound 2a-10 (160 mg, yield 85.26%).

Step J: 2M HCl in EA (1.0 mL) was added to a solution of compound 2a-10 (160 mg, 281.35 μmol, 1 eq) in DCM (1.0 mL). The mixture was stirred at 25° C. for 1 h and concentrated under reduced pressure to afford compound 2a-11 (150 mg, yield 98.46%).

Step K: A solution of intermediate a (2.0 g, 2.37 mmol, 1 eq) in DCM (5 mL) was cooled to 0° C. under nitrogen atmosphere, followed by addition of Dess-Martin periodinane (1.51 g, 3.55 mmol, 1.5 eq). The mixture was warmed to 25° C. and stirred for 1 h, then quenched with water, and extracted with EA. The combined organic layer was washed with water and brine, then dried over Na₂SO₄ and filtered. The filtrate was concentrated under reduced pressure and the residue was purified by column chromatography (EA/PE=0%-20%) to afford compound 2a-12 (1.9 g, yield 95.23%). ¹H NMR (400 MHz, Chloroform-d) δ 9.30 (s, 1H), 9.12 (s, 1H), 7.83 (dd, J=9.1, 5.6 Hz, 1H), 7.55 (d, J=2.6 Hz, 1H), 7.38-7.31 (m, 2H), 5.40-5.30 (m, 2H), 4.85 (d, J=11.9 Hz, 2H), 4.64 (d, J=11.9 Hz, 1H), 4.44 (s, 2H), 4.22 (d, J=12.2 Hz, 1H), 3.84 (s, 1H), 3.55 (s, 4H), 2.13 (s, 1H), 2.05 (d, J=3.8 Hz, 2H), 1.56 (s, 9H), 1.47-1.42 (m, 2H), 1.36 (dd, J=9.0, 5.8 Hz, 2H), 0.91 (t, J=7.5 Hz, 18H), 0.58 (hept, J=7.4 Hz, 3H).

Step L: Et₃N (14.95 mg, 147.74 μmol, 1 eq) was added to a solution of compound 2a-11 (80 mg, 147.74 μmol, 1 eq) in THF (1 mL) and MeOH (1 mL), followed by addition of AcOH (8.7 mg, 147.74 μmol, 1 eq). The mixture was stirred at 25° C. for 10 min, followed by addition of compound 2a-12 (36.85 mg, 162.52 μmol, 1.1 eq) and NaBH₃CN (27.85 mg, 443.2 μmol, 3 eq). The reaction mixture was stirred at 25° C. for 4 h, then warmed to 50° C. and stirred for 16 h and cooled to 25° C. The reaction mixture was quenched with water, and extracted with EA. The combined organic layer was washed with water and brine, then dried over Na₂SO₄ and filtered. The filtrate was concentrated under reduced pressure and the residue was purified by column chromatography (MeOH/DCM=0%-10%) to afford compound 2a-13 (50 mg, yield 26.14%).

Step M: 1M TBAF in THF (0.5 mL) was added to a solution of compound 2a-13 (50 mg, 38.62 μmol, 1 eq) in THF (0.5 mL). The mixture was stirred at 25° C. for 1 h, then quenched with water. The mixture was extracted with EA, and the combined organic layer was washed with water and brine, then dried over Na₂SO₄ and filtered. The filtrate was concentrated under reduced pressure and the residue was purified by column chromatography (MeOH/DCM=0%-6%) to afford compound 2a-14 (25 mg, yield 56.87%).

Step N: 2M HCl in EA (1 mL) was added to a solution of compound 2a-14 (20 mg, 17.57 μmol, 1 eq) in DCM (1 mL). The mixture was stirred at 25° C. for 15 min, then concentrated under reduced pressure. The residue was purified by pre-HPLC (0.1% TFA in water/acetonitrile) to afford compound 2a (5.6 mg, yield 20.10%). ¹H NMR (400 MHz, CD₃OD) δ 9.12 (s, 1H), 7.90 (t, J=7.5 Hz, 1H), 7.49-7.33 (m, 4H), 7.26 (d, J=2.5 Hz, 1H), 4.55 (d, J=4.7 Hz, 2H), 4.33 (d, J=9.5 Hz, 2H), 4.10-3.94 (m, 7H), 3.87 (d, J=11.0 Hz, 1H), 3.79 (s, 2H), 3.65 (d, J=12.8 Hz, 2H), 3.45 (s, 2H), 3.21-2.97 (m, 4H), 2.90 (t, J=6.7 Hz, 2H), 2.60 (s, 1H), 2.15 (d, J=37.5 Hz, 15H), 1.34 (d, J=10.1 Hz, 1H), 1.12-0.83 (m, 6H). m/z (ESI): 994.36 [M+H]⁺.

### Example 141. Synthesis of Compound 3a

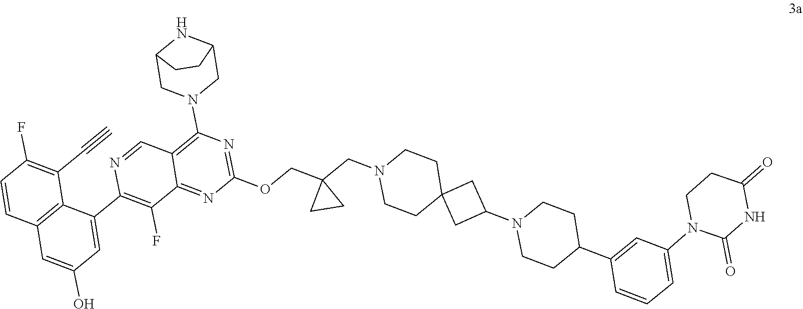 [SMILES: C#Cc1c(F)ccc2cc(O)cc(-c3ncc4c(N5CC6CCC(C5)N6)nc(OCC5(CN6CCC7(CC6)CC(N6CCC(c8cccc(N9CCC(=O)NC9=O)c8)CC6)C7)CC5)nc4c3F)c12]

Compound 3a was synthesized according to the procedure of compound 1a with 3-bromoaniline as start material. ¹H NMR (400 MHz, CD₃OD) δ 0.83 (s, 2H), 0.95 (s, 2H), 1.91-2.12 (m, 15H), 2.29-2.33 (m, 2H), 2.71-2.82 (m, 5H), 3.11-3.24 (m, 3H), 3.45-3.57 (m, 5H), 3.87-3.94 (m, 4H), 4.11 (s, 2H), 4.44-4.51 (m, 2H), 4.75 (d, J=13.6 Hz, 3H), 7.19-7.41 (m, 7H), 7.86-7.89 (m, 1H), 8.48 (s, 1H), 9.08 (s, 1H). m/z (ESI): 922.59 [M+H]⁺.

### Example 142. Synthesis of Compound 4a

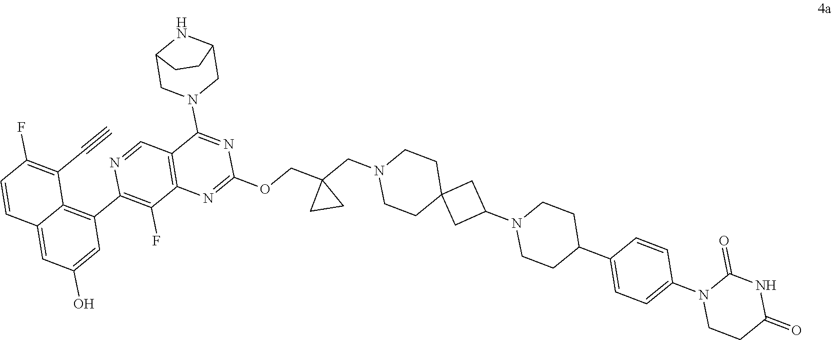 [SMILES: C#Cc1c(F)ccc2cc(O)cc(-c3ncc4c(N5CC6CCC(C5)N6)nc(OCC5(CN6CCC7(CC6)CC(N6CCC(c8ccc(N9CCC(=O)NC9=O)cc8)CC6)C7)CC5)nc4c3F)c12]

Compound 4a was synthesized according to the procedure of compound 1a with 4-bromoaniline as start material. ¹H NMR (400 MHz, CD₃OD) δ ppm 0.88 (d, J=7.3 Hz, 2H), 1.00 (s, 2H), 1.85-2.32 (m, 15H), 2.52 (d, J=7.8 Hz, 1H), 2.80 (t, J=6.7 Hz, 2H), 2.84-3.12 (m, 5H), 3.16-3.26 (m, 1H), 3.42 (q, J=15.2 Hz, 2H), 3.56 (d, J=10.1 Hz, 2H), 3.70 (d, J=8.5 Hz, 2H), 3.85 (t, J=6.6 Hz, 3H), 3.91 (d, J=14.3 Hz, 1H), 4.00 (d, J=14.0 Hz, 1H), 4.28 (d, J=10.8 Hz, 2H), 4.45 (t, J=10.9 Hz, 1H), 4.55 (t, J=10.7 Hz, 1H), 4.80 (d, J=22.8 Hz, 2H), 7.22 (s, 1H), 7.27-7.44 (m, 6H), 7.87 (dd, J=8.9, 5.8 Hz, 1H), 9.08 (s, 1H). m/z (ESI): 922.58 [M+H]⁺.

### Example 143. Synthesis of Compound 5a

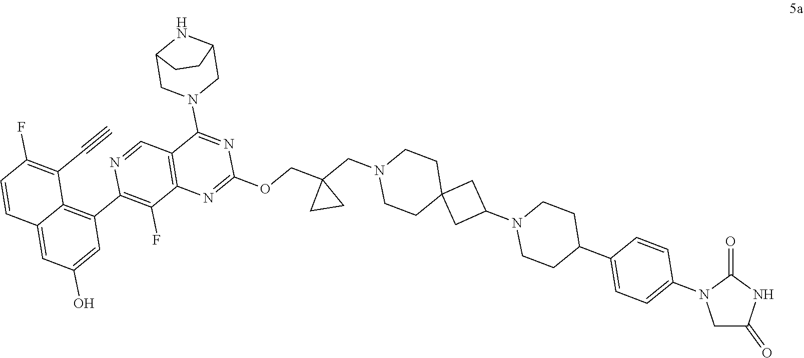 [SMILES: C#Cc1c(F)ccc2cc(O)cc(-c3ncc4c(N5CC6CCC(C5)N6)nc(OCC5(CN6CCC7(CC6)CC(N6CCC(c8ccc(N9CC(=O)NC9=O)cc8)CC6)C7)CC5)nc4c3F)c12]

Compound 5a was synthesized according to the procedure of compound 1a with 4-bromoaniline and ethyl bromoacetate as start materials. ¹H NMR (400 MHz, CD₃OD) δ ppm 0.88 (d, J=6.6 Hz, 2H), 0.97-1.07 (m, 2H), 1.35-1.47 (m, 1H), 1.65 (d, J=6.8 Hz, 1H), 2.06 (dd, J=48.8, 22.4 Hz, 15H), 2.53 (s, 1H), 2.89 (d, J=9.5 Hz, 3H), 2.97-3.14 (m, 2H), 3.41 (dd, J=32.9, 15.2 Hz, 2H), 3.49-3.61 (m, 2H), 3.73 (s, 2H), 3.84 (d, J=11.8 Hz, 1H), 3.92 (d, J=14.3 Hz, 1H), 4.00 (d, J=14.1 Hz, 1H), 4.28 (d, J=9.5 Hz, 2H), 4.43 (s, 2H), 4.45-4.58 (m, 2H), 7.22 (s, 1H), 7.28 (d, J=8.3 Hz, 2H), 7.31-7.40 (m, 2H), 7.58 (d, J=8.4 Hz, 2H), 7.82-7.95 (m, 1H), 9.07 (s, 1H). m/z (ESI): 908.68 [M+H]⁺.

### Example 144. Synthesis of Compound 6a

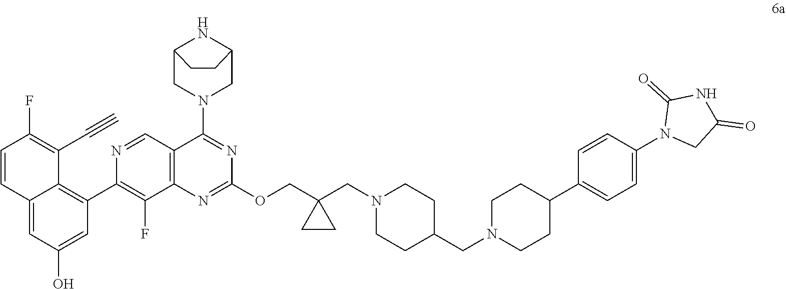 [SMILES: C#Cc1c(F)ccc2cc(O)cc(-c3ncc4c(N5CC6CCC(C5)N6)nc(OCC5(CN6CCC(CN7CCC(c8ccc(N9CC(=O)NC9=O)cc8)CC7)CC6)CC5)nc4c3F)c12]

Compound 6a was synthesized according to the procedure of compound 5a with N-BOC-4-piperidine carboxyaldehyde as start material. ¹H NMR (400 MHz, CD₃OD) δ ppm 0.90 (s, 2H), 1.02 (s, 2H), 1.41 (dd, J=14.4, 7.4 Hz, 1H), 1.65 (s, 1H), 1.76 (d, J=12.7 Hz, 2H), 2.09 (d, J=38.6 Hz, 11H), 2.27 (s, 1H), 2.87 (s, 1H), 3.08 (t, J=15.9 Hz, 6H), 3.19-3.25 (m, 2H), 3.39-3.51 (m, 3H), 3.68 (s, 2H), 3.91 (d, J=13.1 Hz, 2H), 4.02 (d, J=13.3 Hz, 2H), 4.28 (d, J=11.6 Hz, 2H), 4.43 (s, 2H), 4.61 (d, J=11.4 Hz, 1H), 7.22 (s, 1H), 7.27 (d, J=8.0 Hz, 2H), 7.31-7.39 (m, 2H), 7.57 (d, J=7.9 Hz, 2H), 7.79-7.94 (m, 1H), 9.07 (s, 1H). m/z (ESI): 882.64 [M+H]⁺.

### Example 145. Synthesis of Compound 7a

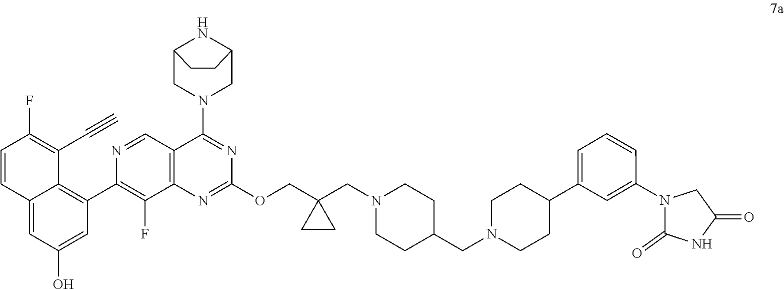 [SMILES: C#Cc1c(F)ccc2cc(O)cc(-c3ncc4c(N5CC6CCC(C5)N6)nc(OCC5(CN6CCC(CN7CCC(c8cccc(N9CC(=O)NC9=O)c8)CC7)CC6)CC5)nc4c3F)c12]

Compound 7a was synthesized according to the procedure of compound 6a with 3-bromoaniline as start material. ¹H NMR (400 MHz, CD₃OD) δ ppm 0.81-0.93 (m, 2H), 0.98-1.04 (m, 2H), 1.41 (dd, J=15.4, 8.1 Hz, 1H), 1.56-1.69 (m, 1H), 1.70-1.88 (m, 2H), 2.01-2.25 (m, 12H), 2.84-2.94 (m, 1H), 2.96-3.15 (m, 6H), 3.16-3.27 (m, 3H), 3.40-3.51 (m, 3H), 3.62-3.73 (m, 2H), 3.84-3.94 (m, 2H), 4.03 (t, J=15.1 Hz, 2H), 4.28 (d, J=13.3 Hz, 2H), 4.39 (d, J=11.7 Hz, 1H), 4.44 (s, 2H), 4.62 (d, J=11.7 Hz, 1H), 7.05 (d, J=7.3 Hz, 1H), 7.22 (d, J=2.5 Hz, 1H), 7.35 (q, J=7.4 Hz, 4H), 7.63 (s, 1H), 7.88 (dd, J=9.1, 5.8 Hz, 1H), 9.07 (s, 1H). m/z (ESI): 882.63 (M+H)+.

### Example 146. Synthesis of Compound 8a

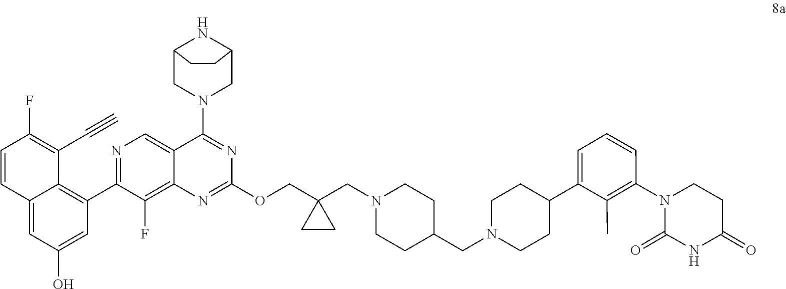 [SMILES: C#Cc1c(F)ccc2cc(O)cc(-c3ncc4c(N5CC6CCC(C5)N6)nc(OCC5(CN6CCC(CN7CCC(c8cccc(N9CCC(=O)NC9=O)c8C)CC7)CC6)CC5)nc4c3F)c12]

Compound 8a was synthesized according to the procedure of compound 6a with 3-bromo-2-methylaniline as start material. ¹H NMR (400 MHz, CD₃OD) δ ppm 9.11 (s, 1H), 7.91 (dd, J=9.1, 5.9 Hz, 1H), 7.44-7.15 (m, 6H), 4.82 (d, J=16.3 Hz, 1H), 4.66 (d, J=11.8 Hz, 1H), 4.44 (d, J=11.9 Hz, 1H), 4.32 (d, J=13.1 Hz, 2H), 4.18-4.00 (m, 2H), 3.99-3.82 (m, 3H), 3.81-3.62 (m, 3H), 3.54-3.44 (m, 2H), 3.28-3.01 (m, 8H), 2.96-2.81 (m, 2H), 2.29 (s, 4H), 2.24-1.99 (m, 10H), 1.92-1.74 (m, 2H), 1.39-1.28 (m, 1H), 1.13-1.02 (m, 2H), 0.98-0.85 (m, 2H). m/z (ESI): 910.64 [M+H]⁺.

### Example 147. Synthesis of Compound 9a

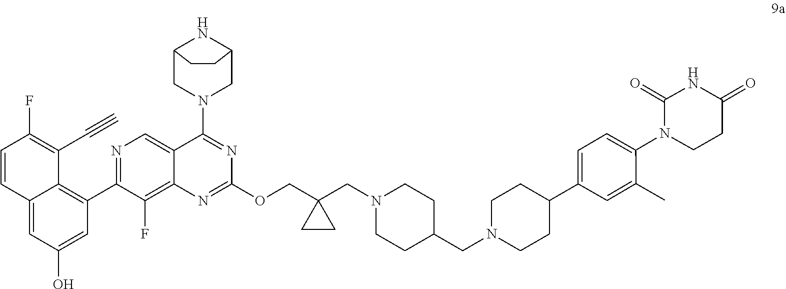 [SMILES: C#Cc1c(F)ccc2cc(O)cc(-c3ncc4c(N5CC6CCC(C5)N6)nc(OCC5(CN6CCC(CN7CCC(c8ccc(N9CCC(=O)NC9=O)c(C)c8)CC7)CC6)CC5)nc4c3F)c12]

Compound 9a was synthesized according to the procedure of compound 6a with 4-bromo-2-methylaniline as start material. ¹H NMR (400 MHz, CD₃OD) δ ppm 9.07 (s, 1H), 7.87 (dd, J=9.1, 5.8 Hz, 1H), 7.40-7.28 (m, 2H), 7.24-7.09 (m, 4H), 4.77 (d, J=13.3 Hz, 1H), 4.62 (d, J=11.8 Hz, 1H), 4.39 (d, J=11.8 Hz, 1H), 4.28 (d, J=13.3 Hz, 2H), 4.13-3.97 (m, 2H), 3.96-3.77 (m, 3H), 3.73-3.58 (m, 3H), 3.53-3.37 (m, 3H), 3.27-3.16 (m, 1H), 3.14-2.96 (m, 5H), 2.94-2.73 (m, 3H), 2.38-2.23 (m, 4H), 2.23-1.97 (m, 10H), 1.90-1.68 (m, 2H), 1.08-0.82 (m, 4H). m/z (ESI): 910.64 [M+H]⁺.

### Example 148. Synthesis of Compound 10a

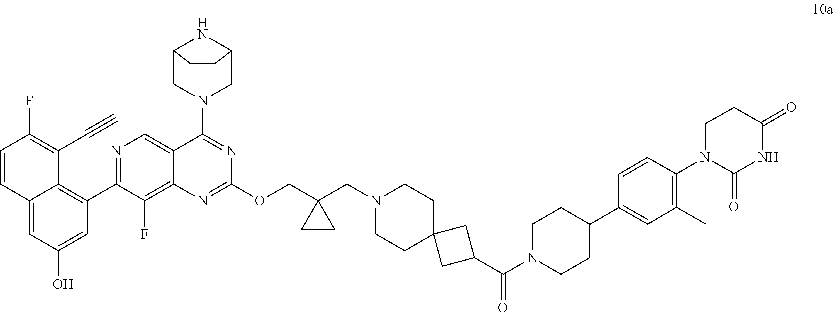 [SMILES: C#Cc1c(F)ccc2cc(O)cc(-c3ncc4c(N5CC6CCC(C5)N6)nc(OCC5(CN6CCC7(CC6)CC(C(=O)N6CCC(c8ccc(N9CCC(=O)NC9=O)c(C)c8)CC6)C7)CC5)nc4c3F)c12]

Compound 10a was synthesized according to the procedure of compound 9a with 7-(tert-Butoxycarbonyl)-7-azaspiro[3.5]nonane-2-carboxylic acid as start material. ¹H NMR (400 MHz, CD₃OD) δ ppm 9.07 (s, 1H), 7.92-7.78 (m, 1H), 7.40-7.24 (m, 2H), 7.24-7.08 (m, 4H), 4.80-4.77 (m, 1H), 4.63 (d, J=13.1 Hz, 1H), 4.59-4.38 (m, 2H), 4.35-4.19 (m, 2H), 4.03-3.54 (m, 8H), 3.48-3.36 (m, 3H), 3.25-2.90 (m, 4H), 2.89-2.63 (m, 4H), 2.29-2.05 (m, 12H), 1.98-1.78 (m, 5H), 1.64-1.46 (m, 2H), 1.03-0.79 (m, 4H). m/z (ESI): 964.61 [M+H]⁺.

### Example 149. Synthesis of Compound 11a

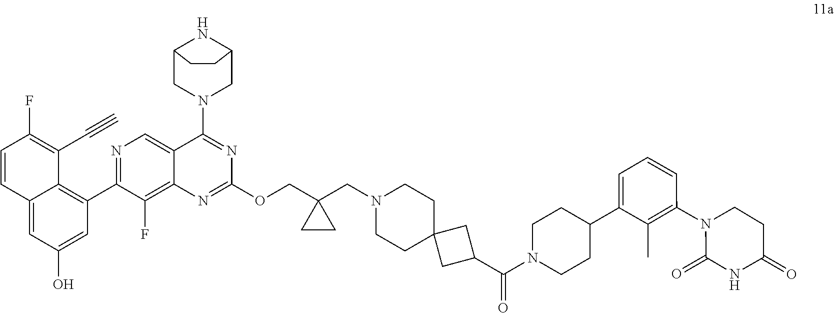 [SMILES: C#Cc1c(F)ccc2cc(O)cc(-c3ncc4c(N5CC6CCC(C5)N6)nc(OCC5(CN6CCC7(CC6)CC(C(=O)N6CCC(c8cccc(N9CCC(=O)NC9=O)c8C)CC6)C7)CC5)nc4c3F)c12]

Compound 11a was synthesized according to the procedure of compound 10a with 3-bromo-2-methylaniline as start material. ¹H NMR (400 MHz, CD₃OD) δ ppm 9.06 (s, 1H), 7.93-7.76 (m, 1H), 7.39-7.07 (m, 6H), 4.80 (d, J=12.8 Hz, 1H), 4.65 (d, J=13.3 Hz, 1H), 4.57-4.38 (m, 2H), 4.26 (d, J=9.1 Hz, 2H), 4.04-3.56 (m, 7H), 3.49-3.34 (m, 3H), 3.23-2.67 (m, 8H), 2.32-2.03 (m, 12H), 1.98-1.74 (m, 5H), 1.66-1.46 (m, 2H), 1.30-1.24 (m, 1H), 1.01-0.78 (m, 4H). m/z (ESI): 964.29 [M+H]⁺.

### Example 150. Synthesis of Compound 12a

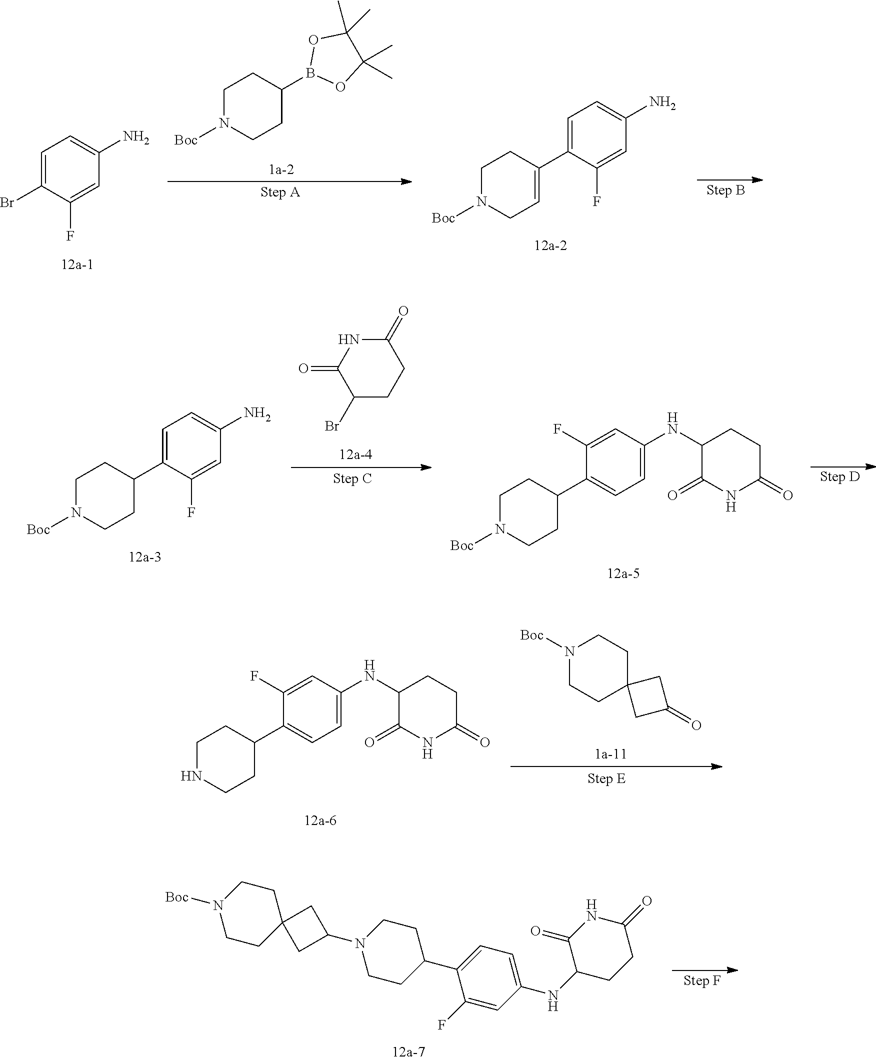

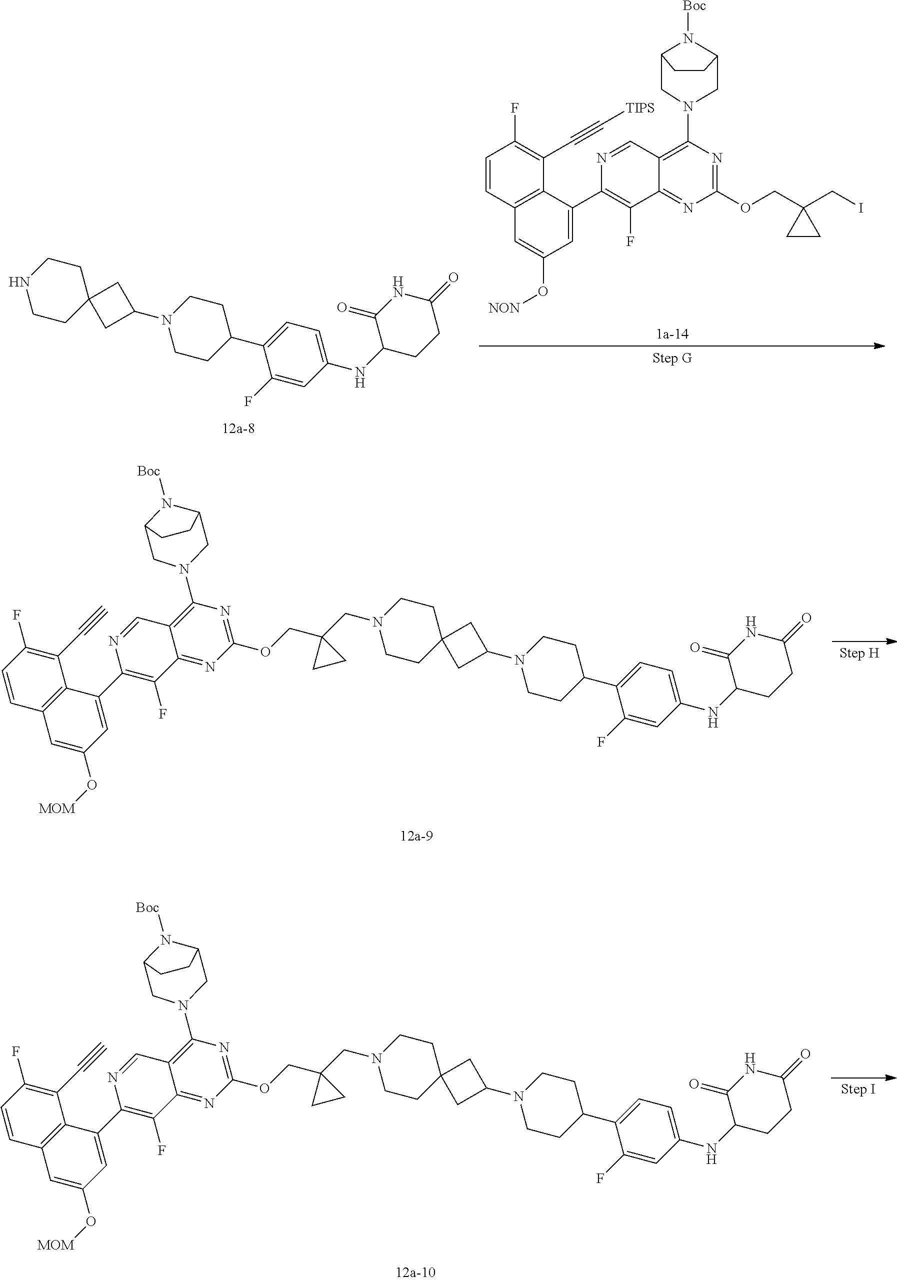

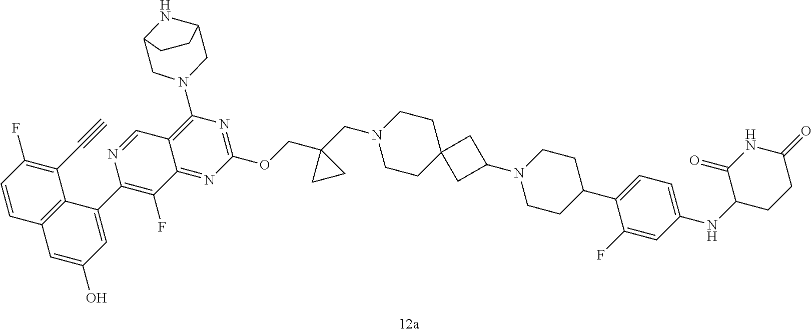 [SMILES: C#Cc1c(F)ccc2cc(O)cc(-c3ncc4c(N5CC6CCC(C5)N6)nc(OCC5(CN6CCC7(CC6)CC(N6CCC(c8ccc(NC9CCC(=O)NC9=O)cc8F)CC6)C7)CC5)nc4c3F)c12]

Step A: Compound 12a-2 (2.44 g, 7.89 mmol, 1 eq) was added to a solution of compound 12a-1 (1.5 g, 7.89 mmol, 1 eq) in dioxane (15 mL) and water (10 mL), followed by addition of K₂CO₃ (1.81 g, 13.10 mmol, 1.66 eq) and PdCl₂(dppf) (462.10 mg, 631.54 μmol, 0.08 eq). The mixture was warmed to 90° C. under the nitrogen atmosphere and stirred for 16 h, then cooled to 25° C. The reaction mixture was quenched with water and extracted with DCM. The combined organic layer was washed with water and brine, then dried over Na₂SO₄ and filtered. The filtrate was concentrated under reduced pressure and the residue was purified by column chromatography (MeOH/DCM=0%-2%) to afford compound 12a-2 (2 g, yield 86.7%).

Step B: 10% Pd/C (1 g) was added to a solution of compound 12a-2 (2 g, 6.84 mmol, 1 eq) in EA (20 mL). The mixture was stirred at 25° C. under hydrogen atmosphere for 15 h, then filtered. The filtrate was concentrated under reduced pressure to afford compound 12a-3 (2 g, yield 99%).

Step C: Compound 12a-4 (1.97 g, 10.24 mmol, 1.5 eq) was added to a solution of compound 12a-3 (2.01 g, 6.83 mmol, 1 eq) in DMF (20 mL), followed by addition of NaHCO₃ (1.72 g, 20.48 mmol, 3 eq). The mixture was warmed to 70° C. and stirred for 16 h, then cooled to 25° C. The reaction mixture was quenched with water, and extracted with EA. The combined organic layer was washed with water and brine, then dried over Na₂SO₄ and filtered. The filtrate was concentrated under reduced pressure and the residue was purified by column chromatography (MeOH/DCM=0%-3%) to afford compound 12a-5 (1.22 g, yield 44.1%).

Step D: TFA (2.06 g, 18.05 mmol, 6 eq) was added to a solution of compound 12a-5 (1.22 g, 3.01 mmol, 1 eq) in DCM (12 mL). The mixture was stirred at 25° C. for 2 h, then concentrated under reduced pressure to afford compound 12a-6 (1.6 g, yield 99%).

Step E: DIPEA (84.65 mg, 654.99 μmol, 1 eq) was added to a solution of compound 12a-6 (200 mg, 654.99 μmol, 1 eq) in DMF (5 mL) and MeOH (1 mL), followed by addition of AcOH (8.7 mg, 147.74 μmol, 1 eq). The mixture was stirred at 25° C. for 10 min, followed by addition of compound 1-11 (203.77 mg, 851.49 μmol, 1.3 eq) and NaBH₃CN (123.48 mg, 1.96 mmol, 3 eq). The reaction mixture was stirred at 50° C. for 16 h, then cooled to 25° C. The reaction mixture was quenched with water, and extracted with EA. The combined organic layer was washed with water and brine, then dried over Na₂SO₄ and filtered. The filtrate was concentrated under reduced pressure to afford compound 12a-7 (346 mg, yield 99%).

Step F: 4M HCl in dioxane (0.5 mL) was added to a solution of compound 12a-7 (80 mg, 151.33 μmol, 1 eq) in DCM (2 mL). The mixture was stirred at 25° C. for 10 min, then concentrated under reduced pressure to afford compound 12a-8 (70 mg, yield 99%).

Step G: Compound 1a-14 (80 mg, 83.86 μmol, 1.3 eq) was added to a solution of compound 12a-8 (0.00 mg, 64.51 μmol, 1 eq) in DMF (3 mL). The mixture was stirred at 50° C. for 4 h, then cooled to 25° C. and quenched with water. The mixture was extracted with EA. The combined organic layer was washed with water and brine, then dried over Na₂SO₄ and filtered. The filtrate was concentrated under reduced pressure and the residue was purified by column chromatography (DCM/MeOH=5/1) to afford compound 12a-9 (20 mg, yield 24.7%).

Step H: TBAF (4.17 mg, 15.94 μmol, 1 eq) was added to a solution of compound 12a-9 (20 mg, 15.94 μmol, 1 eq) in THF (2 mL). The mixture was stirred at 25° C. for 10 min, then quenched with water. The mixture was extracted with EA. The combined organic layer was washed with water and brine, then dried over Na₂SO₄ and filtered. The filtrate was concentrated under reduced pressure to afford compound 12a-10 (17 mg, yield 97%).

Step I: TFA (0.5 mL) was added to a solution of compound 12a-10 (60 mg, 54.63 μmol, 1 eq) in DCM (1 mL). The mixture was stirred at 25° C. for 15 min, then concentrated under reduced pressure. The residue was purified by pre-HPLC (0.1% TFA in water/acetonitrile) to afford compound 12a (6.5 mg, yield 8.4%). ¹H NMR (400 MHz, CD₃OD) δ 0.88-0.93 (m, 2H), 1.00-1.14 (m, 2H), 1.28-1.44 (m, 2H), 1.58-1.70 (m, 2H), 1.93-2.29 (m, 10H), 2.47-2.50 (m, 1H), 2.69-3.25 (m, 9H), 3.39-4.15 (m, 13H), 4.27-4.32 (m, 3H), 4.43-4.57 (m, 2H), 6.46-6.54 (m, 2H), 7.01 (d, J=8.5 Hz, 1H), 7.22 (s, 1H), 7.32-7.37 (m, 2H), 7.86-7.90 (m, 1H), 9.08 (s, 1H). m/z (ESI): 954.57 [M+H]⁺.

### Example 151. Synthesis of Compound 13a

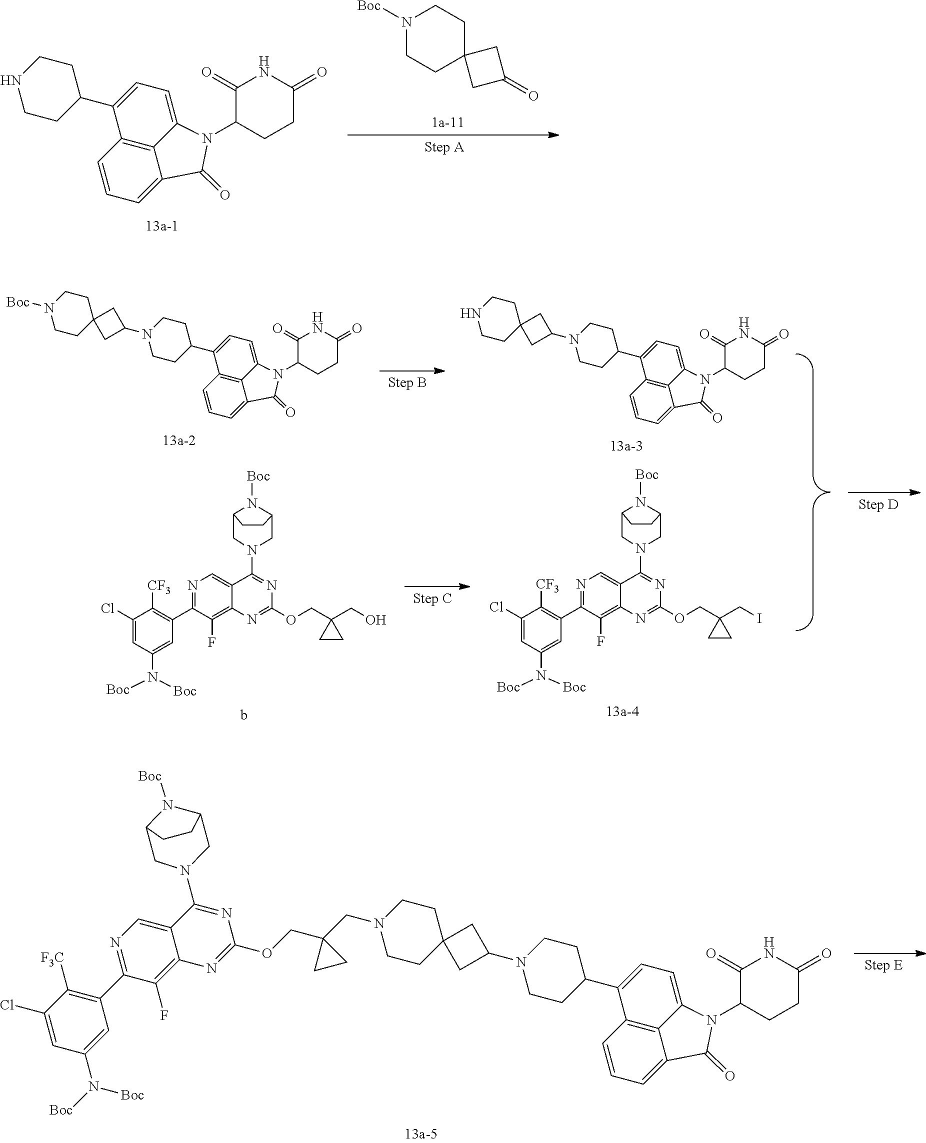

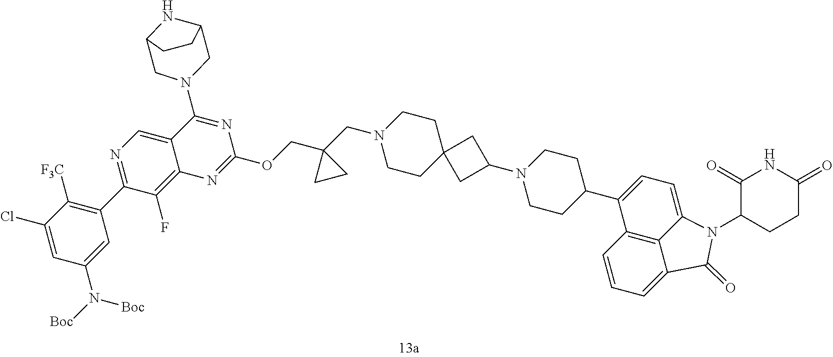 [SMILES: CC(C)(C)OC(=O)N(C(=O)OC(C)(C)C)c1cc(Cl)c(C(F)(F)F)c(-c2ncc3c(N4CC5CCC(C4)N5)nc(OCC4(CN5CCC6(CC5)CC(N5CCC(c7ccc8c9c(cccc79)C(=O)N8C7CCC(=O)NC7=O)CC5)C6)CC4)nc3c2F)c1]

Step A: DIPEA (751.45 mg, 5.81 mmol, 1.5 eq) was added to a solution of compound 13a-1 (1.55 g, 3.88 mmol, 1 eq) in DMF (3.6 mL), followed by addition of compound 1a-11 (1.30 g, 5.43 mmol, 1.4 eq) and AcOH (465.54 mg, 7.75 mmol, 2.0 eq). The mixture was stirred at 25° C. for 1 h, followed by addition of NaBH₃CN (974.34 mg, 15.51 mmol, 4.0 eq). The reaction mixture was stirred at 25° C. for 16 h, then quenched with water, and extracted with EA. The combined organic layer was washed with water and brine, then dried over Na₂SO₄ and filtered. The filtrate was concentrated under reduced pressure and the residue was purified by column chromatography (MeOH/DCM=0%-7%) to afford compound 13a-2 (690 mg, yield 30.3%).

Step B: 4M HCl in dioxane (1.5 mL) was added to a solution of compound 13a-2 (180 mg, 306.79 μmol, 1 eq) in DCM (2 mL). The mixture was stirred at 25° C. for 4 h, then concentrated under reduced pressure to afford compound 13a-3 (305.9 mg, yield 99.7%).

Step C: Imidazole (83.77 mg, 1.23 mmol, 3.5 eq) was added to a solution of intermediate b (300 mg, 351.58 μmol, 1 eq) in DCM (15 mL), followed by addition of PPh₃ (221.32 mg, 843.79 μmol, 2.4 eq) and I₂ (214.16 mg, 843.79 μmol, 2.4 eq). The mixture was stirred at 25° C. for 16 h, then quenched with water and extracted with DCM. The combined organic layer was washed with water and brine, then dried over Na₂SO₄ and filtered. The filtrate was concentrated under reduced pressure and the residue was purified by column chromatography (EA/PE=0%-50%) to afford compound 13a-4 (250 mg, yield 73.8%).

Step D: Et₃N (48.36 mg, 477.95 μmol, 5.0 eq) was added to a solution of compound 13a-3 (50 mg, 95.59 μmol, 1 eq) in DMF (5 mL), followed by addition of compound 13a-4 (92.07 mg, 95.59 μmol, 1 eq). The mixture was stirred at 25° C. for 16 h, then quenched with water and extracted with EA. The combined organic layer was washed with water and brine, then dried over Na₂SO₄ and filtered. The filtrate was concentrated under reduced pressure and the residue was purified by column chromatography (MeOH/DCM=0%-15%) to afford compound 13a-5 (30 mg, yield 23.7%).

Step E: TFA (1 mL) was added to a solution of compound 13a-5 (30 mg, 22.69 μmol, 1 eq) in DCM (13 mL). The mixture was stirred at 25° C. for 1 h, then concentrated under reduced pressure. The residue was purified by pre-HPLC (0.1% TFA in water/acetonitrile) to afford compound 13a (16 mg, yield 56.3%). ¹H NMR (400 MHz, CD₃OD) δ ppm 0.89 (t, J=6.7 Hz, 3H), 1.27 (s, 26H), 1.51 (d, J=6.8 Hz, 2H), 1.82 (p, J=6.4 Hz, 2H), 3.39 (t, J=6.6 Hz, 2H), 3.48 (t, J=6.3 Hz, 2H), 3.67 (dd, J=13.1, 8.9 Hz, 1H), 3.85 (dd, J=13.2, 8.9 Hz, 1H), 3.92 (q, J=6.4 Hz, 2H), 4.09-4.21 (m, 2H), 4.33 (d, J=7.0 Hz, 1H), 4.46 (dd, J=14.6, 6.3 Hz, 1H), 4.59 (d, J=14.6 Hz, 1H), 8.13 (s, 1H), 8.31 (s, 1H), 8.42 (s, 1H). m/z (ESI): 1020.93 [M+H]⁺.

### Example 152. Synthesis of Compound 14a

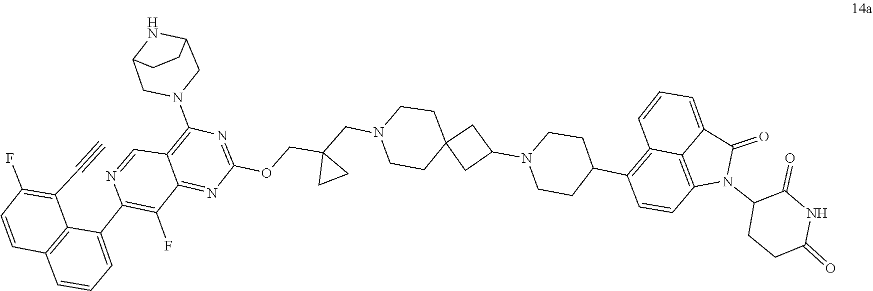 [SMILES: C#Cc1c(F)ccc2cccc(-c3ncc4c(N5CC6CCC(C5)N6)nc(OCC5(CN6CCC7(CC6)CC(N6CCC(c8ccc9c%10c(cccc8%10)C(=O)N9C8CCC(=O)NC8=O)CC6)C7)CC5)nc4c3F)c12]

Compound 14a was synthesized according to the procedure of compound 13a with intermediate c as start material. ¹H NMR (400 MHz, CD₃OD) δ 0.89-1.00 (m, 4H), 1.95-2.24 (m, 22H), 2.81-3.10 (m, 7H), 3.53 (s, 1H), 3.60-4.05 (m, 8H), 4.27-4.30 (m, 2H), 4.44 (t, J=11.2 Hz, 0.5H), 4.57 (t, J=10.8 Hz, 0.5H), 5.41-5.47 (m, 1H), 7.08 (d, J=7.6 Hz, 1H), 7.38-7.47 (m, 2H), 7.66-7.68 (m, 2H), 7.85 (t, J=7.6 Hz, 1H), 8.08-8.14 (m, 4H), 8.41 (d, J=8.0 Hz, 1H), 9.11 (s, 1H). m/z (ESI): 996.50 [M+H]⁺.

### Example 153. Synthesis of Compound 15a

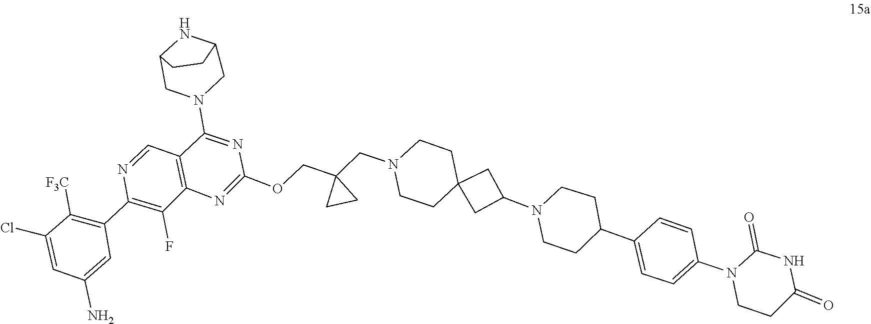 [SMILES: Nc1cc(Cl)c(C(F)(F)F)c(-c2ncc3c(N4CC5CCC(C4)N5)nc(OCC4(CN5CCC6(CC5)CC(N5CCC(c7ccc(N8CCC(=O)NC8=O)cc7)CC5)C6)CC4)nc3c2F)c1]

Compound 15a was synthesized according to the procedure of compound 4a with intermediate b as start material. ¹H NMR (400 MHz, CD₃OD) δ ppm 0.87 (s, 2H), 0.99 (s, 2H), 1.87-2.21 (m, 15H), 2.29 (dd, J=26.1, 11.2 Hz, 2H), 2.55 (s, 1H), 2.80 (t, J=6.7 Hz, 2H), 2.93 (t, J=12.5 Hz, 4H), 3.06 (dd, J=32.4, 16.3 Hz, 2H), 3.58 (t, J=12.0 Hz, 2H), 3.76 (dd, J=22.0, 10.3 Hz, 3H), 3.85 (t, J=6.7 Hz, 2H), 3.92 (d, J=13.7 Hz, 2H), 4.25 (s, 2H), 4.37-4.57 (m, 2H), 4.80 (s, 2H), 6.48 (s, 1H), 6.91 (s, 1H), 7.33 (s, 4H), 9.05 (s, 1H). m/z (ESI): 931.44 [M+H]⁺.

### Example 154. Synthesis of Compound 16a

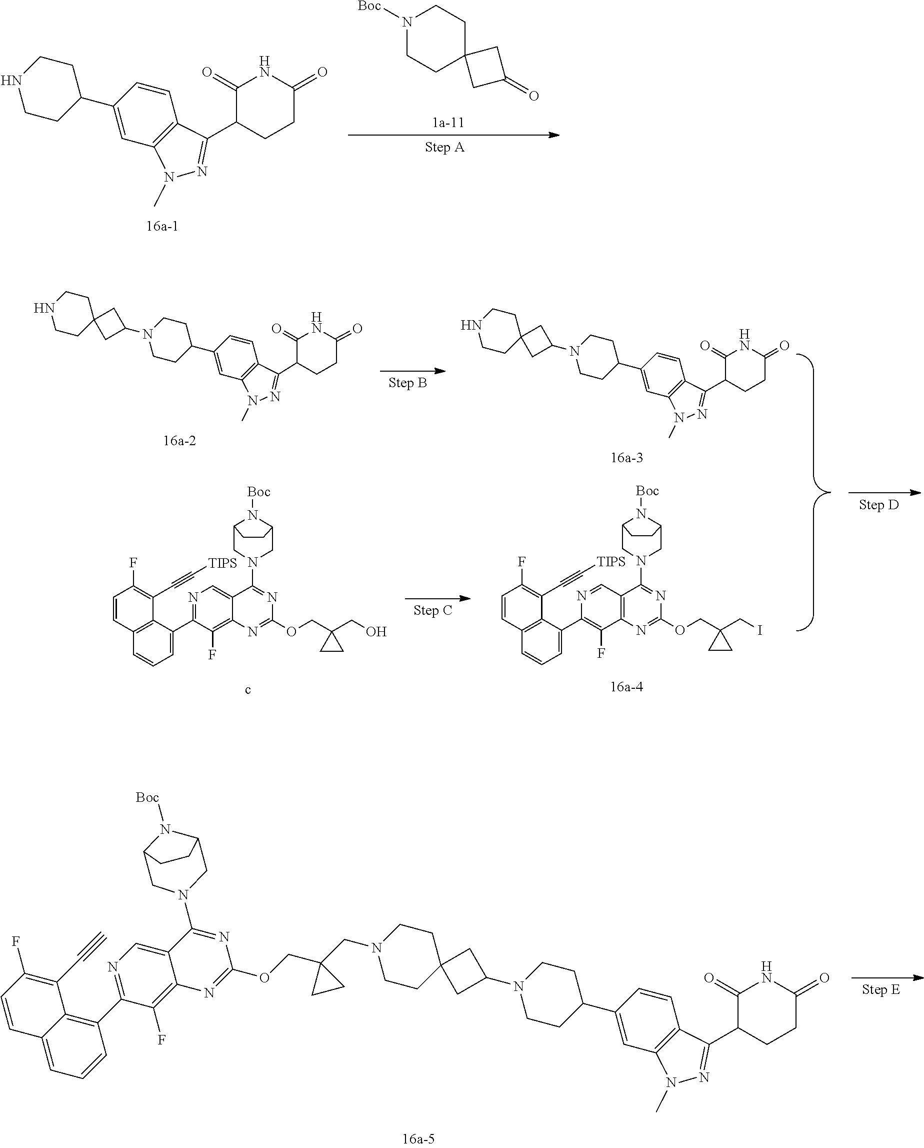

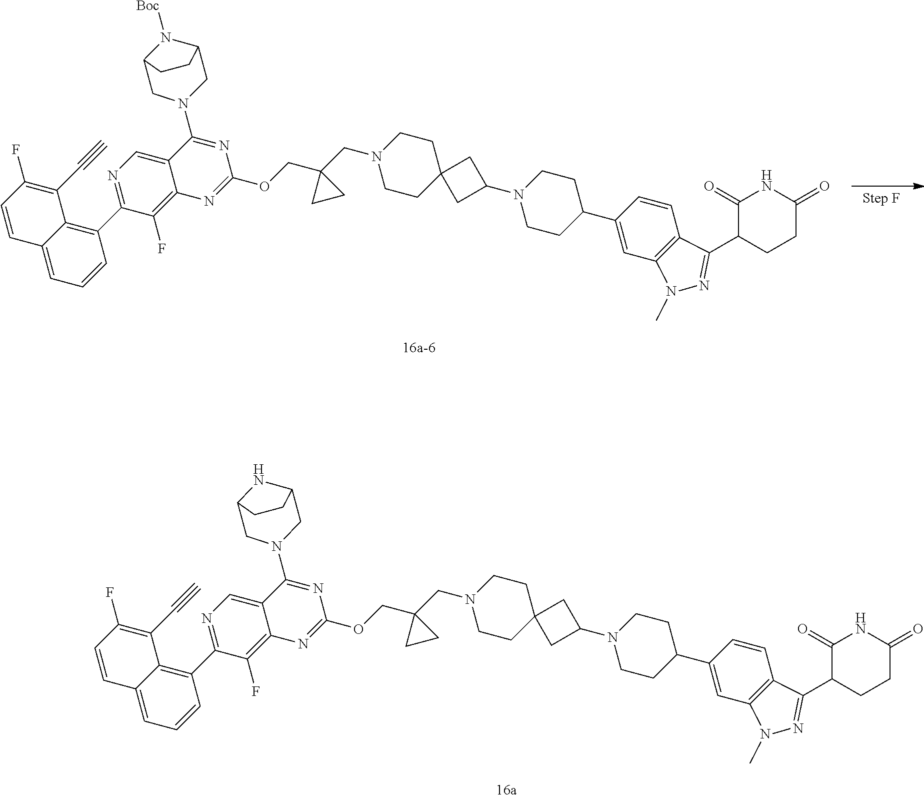 [SMILES: C#Cc1c(F)ccc2cccc(-c3ncc4c(N5CC6CCC(C5)N6C(=O)OC(C)(C)C)nc(OCC5(CN6CCC7(CC6)CC(N6CCC(c8ccc9c(C%10CCC(=O)NC%10=O)nn(C)c9c8)CC6)C7)CC5)nc4c3F)c12 ; C#Cc1c(F)ccc2cccc(-c3ncc4c(N5CC6CCC(C5)N6)nc(OCC5(CN6CCC7(CC6)CC(N6CCC(c8ccc9c(C%10CCC(=O)NC%10=O)nn(C)c9c8)CC6)C7)CC5)nc4c3F)c12]

Step A: Et₃N (420.59 mg, 4.16 mmol, 5.0 eq) was added to a solution of compound 16a-1 (1.1 g, 3.03 mmol, 1 eq) in MeOH (10 mL) and THF (10 mL), followed by addition of compound 1a-11 (1.45 g, 6.06 mmol, 2 eq) and AcOH (14.98 mg, 249.39 μmol, 0.3 eq). The mixture was stirred at 50° C. for 4 h, followed by addition of NaBH₃CN (156.72 mg, 2.49 mmol, 3.0 eq). The reaction mixture was stirred at 50° C. for 16 h, then quenched with water, and extracted with DCM. The combined organic layer was washed with water and brine, then dried over Na₂SO₄ and filtered. The filtrate was concentrated under reduced pressure and the residue was purified by column chromatography (MeOH/DCM=0%-3%) to afford compound 16a-2 (1.5 g, yield 90.1%).

Step B: 4M HCl in dioxane (7 mL) was added to a solution of compound 16a-2 (1.5 g, 2.73 mmol, 1 eq) in DCM (28 mL). The mixture was stirred at 25° C. for 30 min, then concentrated under reduced pressure to afford compound 16a-3 (1.6 g, yield 99.7%).

Step C: Imidazole (199.72 mg, 2.93 mmol, 10 eq) was added to a solution of intermediate c (230 mg, 293.36 μmol, 1 eq) in DCM (5 mL), followed by addition of PPh₃ (769.45 mg, 2.93 mmol, 10 eq) and I₂ (769.45 mg, 2.93 mmol, 10 eq). The mixture was stirred at 25° C. for 2 h, then quenched with water and extracted with DCM. The combined organic layer was washed with water and brine, then dried over Na₂SO₄ and filtered. The filtrate was concentrated under reduced pressure and the residue was purified by column chromatography (EA/PE=3/10) to afford compound 16a-4 (224 mg, yield 85.4%).

Step D: Compound 16a-4 (460 mg, 482.20 μmol, 1 eq) was added to a solution of compound 16a-3 (216.29 mg, 482.20 μmol, 1 eq) in DMF (10 mL), followed by addition of DIPEA (62.32 mg, 482.20 μmol, 1 eq). The mixture was stirred at 43° C. for 16 h, then cooled to 25° C. and concentrated under reduced pressure. The residue was purified by column chromatography (MeOH/DCM=0%-9%) to afford compound 16a-5 (400 mg, yield 65.1%).

Step E: 1M TBAF in THF (0.4 mL) was added to a solution of compound 16a-5 (90 mg, 74.04 μmol, 1 eq) in THF (2 mL). The mixture was stirred at 25° C. for 1 h, then quenched with water and extracted with DCM. The combined organic layer was washed with water and brine, then dried over Na₂SO₄ and filtered. The filtrate was concentrated under reduced pressure and the residue was purified by column chromatography (MeOH/DCM=0%-12%) to afford compound 16a-6 (32 mg, yield 40.8%).

Step F: 4M HCl in dioxane (0.3 mL) was added to a solution of compound 16a-6 (32 mg, 30.21 μmol, 1 eq) in DCM (3 mL). The mixture was stirred at 25° C. for 15 min, then concentrated under reduced pressure. The residue was purified by pre-HPLC (0.1% TFA in water/acetonitrile) to afford compound 16a (10.5 mg, yield 26.3%). ¹H NMR (400 MHz, CD₃OD) δ ppm 9.07 (s, 1H), 8.16-8.03 (m, 2H), 7.74-7.56 (m, 3H), 7.48-7.39 (m, 1H), 7.36 (s, 1H), 7.07 (d, J=8.5 Hz, 1H), 4.60-4.39 (m, 2H), 4.34 (dd, J=9.2, 5.1 Hz, 1H), 4.31-4.19 (m, 2H), 4.00 (s, 3H), 3.97-3.79 (m, 3H), 3.79-3.67 (m, 2H), 3.64-3.40 (m, 5H), 3.14-2.86 (m, 4H), 2.77-2.69 (m, 1H), 2.59-2.36 (m, 2H), 2.36-1.81 (m, 16H), 1.27 (d, J=9.7 Hz, 4H), 1.04-0.93 (m, 2H), 0.93-0.81 (m, 2H). m/z (ESI): 959.62 [M+H]⁺.

### Example 155. Synthesis of Compound 17a

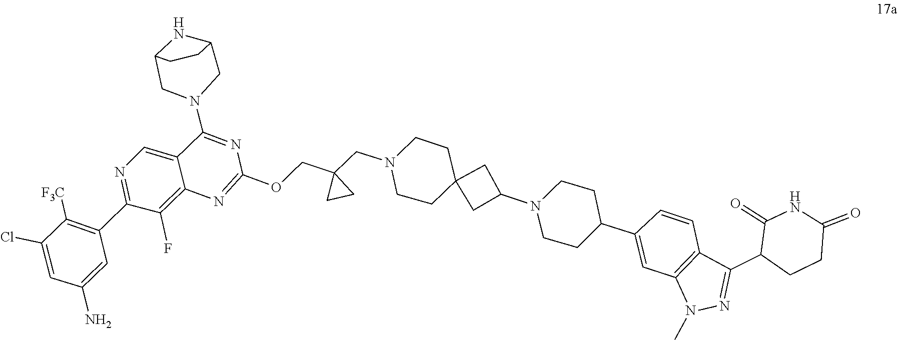 [SMILES: Cn1nc(C2CCC(=O)NC2=O)c2ccc(C3CCN(C4CC5(CCN(CC6(COc7nc(N8CC9CCC(C8)N9)c8cnc(-c9cc(N)cc(Cl)c9C(F)(F)F)c(F)c8n7)CC6)CC5)C4)CC3)cc21]

Compound 17a was synthesized according to the procedure of compound 16a with intermediate b as start material. ¹H NMR (400 MHz, CD₃OD) δ ppm 9.04 (s, 1H), 7.68 (d, J=8.5 Hz, 1H), 7.37 (s, 1H), 7.08 (d, J=8.5 Hz, 1H), 6.89 (s, 1H), 6.47 (d, J=2.3 Hz, 1H), 4.56-4.38 (m, 2H), 4.34 (dd, J=9.3, 5.1 Hz, 1H), 4.24 (s, 2H), 4.00 (s, 3H), 3.90 (d, J=14.1 Hz, 2H), 3.83-3.70 (m, 3H), 3.67-3.43 (m, 3H), 3.15-2.88 (m, 5H), 2.82-2.65 (m, 2H), 2.60-2.38 (m, 2H), 2.35-1.91 (m, 16H), 1.34-1.20 (m, 3H), 1.03-0.81 (m, 4H). m/z (ESI): 984.52 [M+H]⁺.

### Example 156. Synthesis of Compound 18a

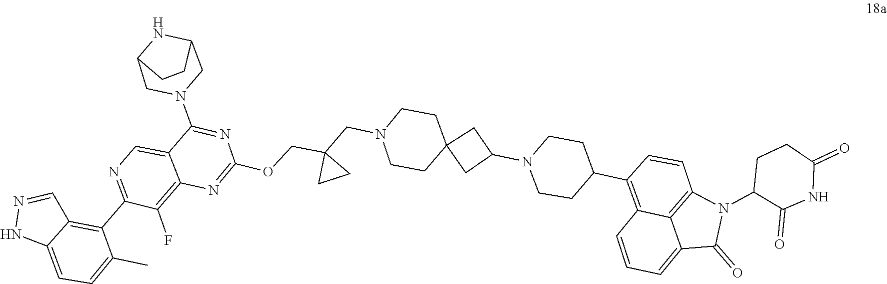 [SMILES: Cc1ccc2[nH]ncc2c1-c1ncc2c(N3CC4CCC(C3)N4)nc(OCC3(CN4CCC5(CC4)CC(N4CCC(c6ccc7c8c(cccc68)C(=O)N7C6CCC(=O)NC6=O)CC4)C5)CC3)nc2c1F]

Compound 18a was synthesized according to the procedure of compound 13a with intermediate d as start material. ¹H NMR (400 MHz, CD₃OD) δ 9.24 (s, 1H), 8.45 (d, J=8.3 Hz, 1H), 8.15 (d, J=6.9 Hz, 1H), 7.90 (t, J=7.7 Hz, 1H), 7.73 (s, 1H), 7.64 (d, J=8.6 Hz, 1H), 7.45 (dd, J=15.2, 8.0 Hz, 2H), 7.11 (d, J=7.4 Hz, 1H), 5.46 (dd, J=12.6, 5.4 Hz, 1H), 4.63-4.47 (m, 2H), 4.31 (s, 2H), 4.00 (d, J=14.1 Hz, 2H), 3.84 (q, J=8.7 Hz, 3H), 3.68 (q, J=15.6, 14.4 Hz, 3H), 3.44 (d, J=38.3 Hz, 2H), 3.18-2.82 (m, 7H), 2.64 (d, J=23.5 Hz, 1H), 2.47-1.88 (m, 21H), 1.04 (s, 2H), 0.93 (s, 2H). m/z (ESI): 958.62 [M+H]⁺.

### Example 157. Synthesis of Compound 19a

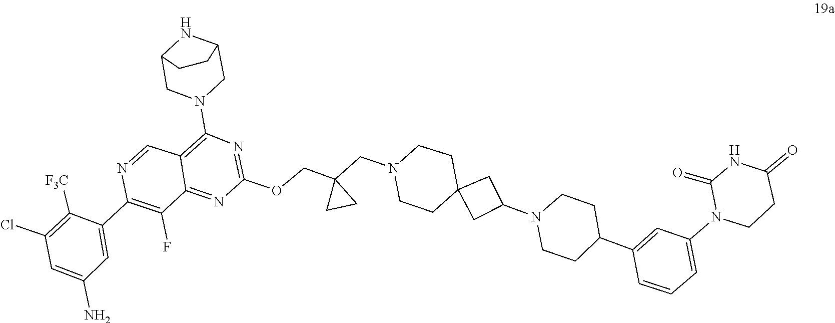 [SMILES: Nc1cc(Cl)c(C(F)(F)F)c(-c2ncc3c(N4CC5CCC(C4)N5)nc(OCC4(CN5CCC6(CC5)CC(N5CCC(c7cccc(N8CCC(=O)NC8=O)c7)CC5)C6)CC4)nc3c2F)c1]

Compound 19a was synthesized according to the procedure of compound 3a with intermediate b as start material. ¹H NMR (400 MHz, CD₃OD) δ 0.88 (s, 2H), 0.99 (s, 2H), 1.96-2.53 (m, 18H), 2.55 (t, J=10.4 Hz, 1H), 2.82 (t, J=6.0 Hz, 2H), 2.90-3.12 (m, 5H), 3.54-3.95 (m, 10H), 4.26 (s, 2H), 4.43-4.52 (m, 2H), 6.49 (s, 1H), 6.91 (s, 1H), 7.22 (t, J=9.2 Hz, 2H), 7.28 (s, 1H), 7.40 (t, J=7.6 Hz, 1H), 9.06 (s, 1H). m/z (ESI): 931.52 [M+H]⁺.

### Example 158. Synthesis of Compound 20a

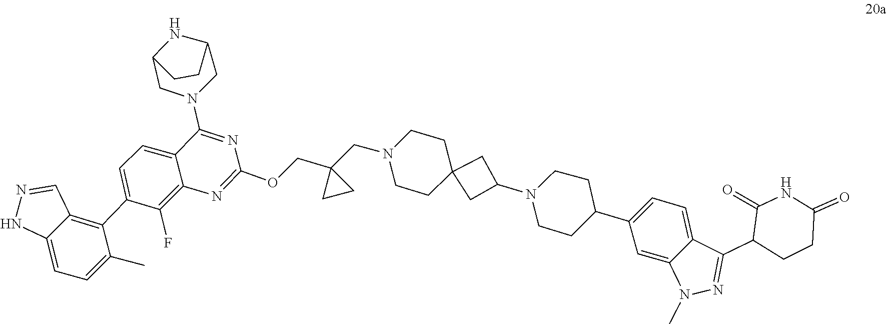 [SMILES: Cc1ccc2[nH]ncc2c1-c1ccc2c(N3CC4CCC(C3)N4)nc(OCC3(CN4CCC5(CC4)CC(N4CCC(c6ccc7c(C8CCC(=O)NC8=O)nn(C)c7c6)CC4)C5)CC3)nc2c1F]

Compound 20a was synthesized according to the procedure of compound 16a with intermediate d as start material. ¹H NMR (400 MHz, CD₃OD) δ 7.95 (d, J=8.7 Hz, 1H), 7.67 (d, J=8.4 Hz, 1H), 7.59-7.52 (m, 2H), 7.44-7.35 (m, 3H), 7.07 (d, J=8.6 Hz, 1H), 4.70 (d, J=17.1 Hz, 4H), 4.48 (d, J=14.4 Hz, 2H), 4.34 (dd, J=9.3, 5.1 Hz, 1H), 4.25 (s, 2H), 3.99 (s, 3H), 3.88 (t, J=12.8 Hz, 2H), 3.74 (t, J=9.0 Hz, 3H), 3.61-3.55 (m, 2H), 3.00 (dq, J=38.8, 12.7, 12.0 Hz, 5H), 2.81-2.65 (m, 2H), 2.56-2.40 (m, 2H), 2.28 (s, 6H), 2.16 (s, 7H), 2.00 (d, J=28.7 Hz, 6H), 0.93 (d, J=45.3 Hz, 4H). m/z (ESI): 920.77 [M+H]⁺.

### Example 159. Synthesis of Compound 21a

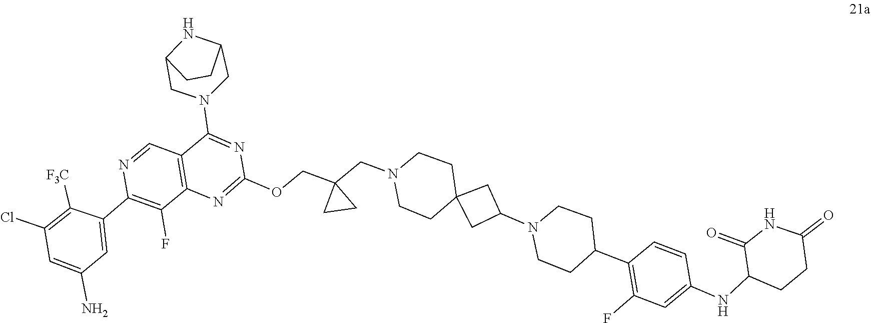 [SMILES: Nc1cc(Cl)c(C(F)(F)F)c(-c2ncc3c(N4CC5CCC(C4)N5)nc(OCC4(CN5CCC6(CC5)CC(N5CCC(c7ccc(NC8CCC(=O)NC8=O)cc7F)CC5)C6)CC4)nc3c2F)c1]

Compound 21a was synthesized according to the procedure of compound 12a with intermediate b as start material. ¹H NMR (400 MHz, CD₃OD) δ 9.07 (s, 1H), 7.05-6.99 (m, 1H), 6.92 (d, J=2.1 Hz, 1H), 6.58-6.45 (m, 3H), 4.53-4.42 (m, 2H), 4.34-4.21 (m, 3H), 3.97-3.88 (m, 2H), 3.83-3.69 (m, 3H), 3.62-3.53 (m, 2H), 3.50-3.45 (m, 1H), 3.14-2.67 (m, 10H), 2.60-2.48 (s, 2H), 2.37-1.87 (m, 15H), 1.32-1.28 (s, 1H), 1.05-0.96 (m, 2H), 0.92-0.84 (m, 2H). m/z (ESI): 963 [M+H]⁺.

### Example 160. Synthesis of Compound 22a

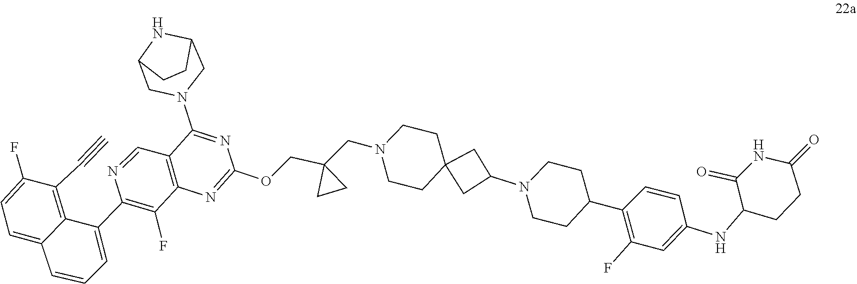 [SMILES: C#Cc1c(F)ccc2cccc(-c3ncc4c(N5CC6CCC(C5)N6)nc(OCC5(CN6CCC7(CC6)CC(N6CCC(c8ccc(NC9CCC(=O)NC9=O)cc8F)CC6)C7)CC5)nc4c3F)c12]

Compound 22a was synthesized according to the procedure of compound 12a with intermediate c as start material. ¹H NMR (400 MHz, CD₃OD) δ 9.11 (s, 1H), 8.16 (dt, J=8.7, 3.9 Hz, 2H), 7.77-7.58 (m, 2H), 7.48 (t, J=8.9 Hz, 1H), 7.02 (t, J=8.5 Hz, 1H), 6.61-6.44 (m, 2H), 4.60-4.42 (m, 2H), 4.34-4.26 (m, 2H), 4.04-3.91 (m, 14.0 Hz, 2H), 3.89-3.89 (m, 1H), 3.79-3.66 (m, 2H), 3.58-3.47 (m, 2H), 3.46-3.38 (m, 1H), 3.28-3.23 (m, 2H), 3.13-2.99 (m, 3H), 2.97-2.86 (m, 2H), 2.85-2.69 (m, 2H), 2.54 (s, 1H), 2.36-1.90 (m, 14H), 1.73-1.58 (m, 2H), 1.48-1.39 (m, 2H), 1.35-1.31 (m, 1H), 1.06-1.01 (m, 3H), 0.94-0.86 (m, 2H). m/z (ESI): 938.5 [M+H]⁺.

### Example 161. Synthesis of Compound 23a

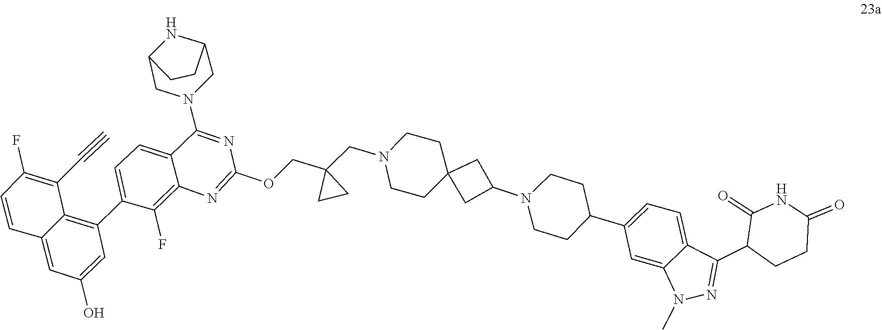 [SMILES: C#Cc1c(F)ccc2cc(O)cc(-c3ccc4c(N5CC6CCC(C5)N6)nc(OCC5(CN6CCC7(CC6)CC(N6CCC(c8ccc9c(C%10CCC(=O)NC%10=O)nn(C)c9c8)CC6)C7)CC5)nc4c3F)c12]

Compound 23a was synthesized according to the procedure of compound 16a with intermediate c as start material. ¹H NMR (400 MHz, CD₃OD) δ 7.95 (d, J=8.7 Hz, 1H), 7.67 (d, J=8.4 Hz, 1H), 7.59-7.52 (m, 2H), 7.44-7.35 (m, 3H), 7.07 (d, J=8.6 Hz, 1H), 4.70 (d, J=17.1 Hz, 4H), 4.48 (d, J=14.4 Hz, 2H), 4.34 (dd, J=9.3, 5.1 Hz, 1H), 4.25 (s, 2H), 3.99 (s, 3H), 3.88 (t, J=12.8 Hz, 2H), 3.74 (t, J=9.0 Hz, 3H), 3.61-3.55 (m, 2H), 3.00 (dq, J=38.8, 12.7, 12.0 Hz, 5H), 2.81-2.65 (m, 2H), 2.56-2.40 (m, 2H), 2.28 (s, 6H), 2.16 (s, 7H), 2.00 (d, J=28.7 Hz, 6H), 0.93 (d, J=45.3 Hz, 4H). m/z (ESI): 974.55 [M+H]⁺.

### Example 162. Synthesis of Compound 24a

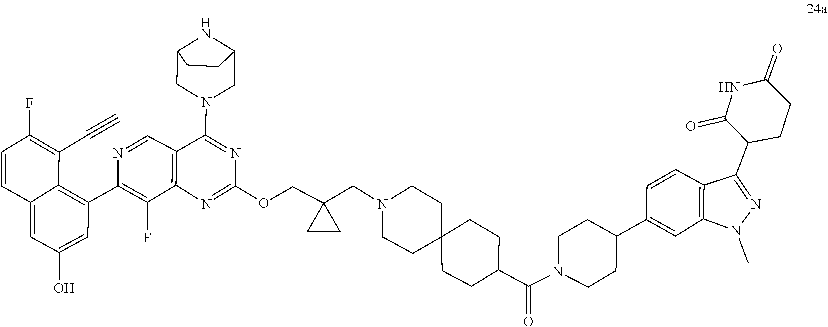 [SMILES: C#Cc1c(F)ccc2cc(O)cc(-c3ncc4c(N5CC6CCC(C5)N6)nc(OCC5(CN6CCC7(CCC(C(=O)N8CCC(c9ccc%10c(C%11CCC(=O)NC%11=O)nn(C)c%10c9)CC8)CC7)CC6)CC5)nc4c3F)c12]

Compound 24a was synthesized according to the procedure of compound 16a with 3-(tert-butoxycarbonyl)-3-azaspiro[5.5]undecane-9-carboxylic acid as start material. ¹H NMR (400 MHz, CD₃OD) δ 0.92 (d, J=9.1 Hz, 2H), 1.05 (s, 2H), 1.33 (s, 3H), 1.47 (q, J=7.3 Hz, 4H), 1.61-1.77 (m, 12H), 1.88 (t, J=13.7 Hz, 2H), 2.20 (s, 4H), 2.34 (d, J=14.3 Hz, 2H), 2.74-2.82 (m, 4H), 3.14-3.20 (m, 4H), 3.27 (d, J=15.9 Hz, 3H), 3.43 (s, 2H), 3.98 (d, J=15.2 Hz, 2H), 4.04 (s, 3H), 4.22 (d, J=14.7 Hz, 1H), 4.32 (s, 2H), 4.39 (s, 1H), 4.59 (d, J=25.2 Hz, 1H), 4.78 (d, J=13.8 Hz, 2H), 7.12 (d, J=8.5 Hz, 1H), 7.25 (s, 1H), 7.39 (d, J=12.3 Hz, 3H), 7.70 (d, J=8.5 Hz, 1H), 7.91 (s, 1H), 9.13 (s, 1H). m/z (ESI): 1032.49 [M+H]⁺.

### Example 163. Synthesis of Compound 25a

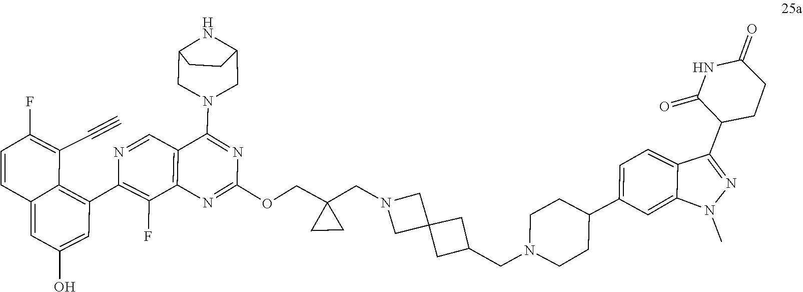 [SMILES: C#Cc1c(F)ccc2cc(O)cc(-c3ncc4c(N5CC6CCC(C5)N6)nc(OCC5(CN6CC7(CC(CN8CCC(c9ccc%10c(C%11CCC(=O)NC%11=O)nn(C)c%10c9)CC8)C7)C6)CC5)nc4c3F)c12]

Compound 25a was synthesized according to the procedure of compound 16a with tert-butyl 6-formyl-2-azaspiro[3.3]heptane-2-carboxylate as start material. ¹H NMR (400 MHz, CD₃OD) δ ppm 0.89 (m, 2H), 1.02 (t, J=7.3 Hz, 2H), 1.41 (dd, J=14.9, 7.4 Hz, 2H), 1.55-1.72 (m, 2H), 1.89-2.37 (m, 13H), 2.47 (s, 2H), 2.64-2.83 (m, 4H), 2.93 (s, 1H), 3.09 (d, J=22.2 Hz, 2H), 3.16-3.25 (m, 3H), 3.46 (d, J=4.2 Hz, 1H), 3.54 (d, J=7.2 Hz, 2H), 3.92 (d, J=14.2 Hz, 1H), 4.01 (s, 3H), 4.12 (dd, J=16.1, 11.7 Hz, 1H), 4.27 (d, J=11.5 Hz, 3H), 4.39 (d, J=8.7 Hz, 2H), 4.43-4.68 (m, 2H), 7.01 (d, J=8.3 Hz, 1H), 7.21 (d, J=11.6 Hz, 2H), 7.27 (d, J=7.9 Hz, 2H), 7.69 (d, J=8.3 Hz, 2H), 9.07 (s, 1H). m/z (ESI): 961.52 [M+H]⁺.

### Example 164. Synthesis of Compound 26a

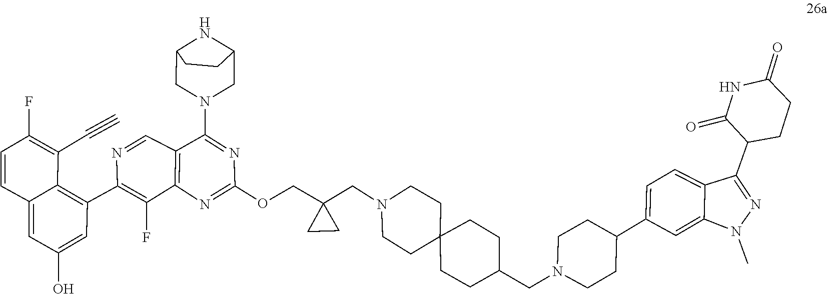 [SMILES: C#Cc1c(F)ccc2cc(O)cc(-c3ncc4c(N5CC6CCC(C5)N6)nc(OCC5(CN6CCC7(CCC(CN8CCC(c9ccc%10c(C%11CCC(=O)NC%11=O)nn(C)c%10c9)CC8)CC7)CC6)CC5)nc4c3F)c12]

Compound 26a was synthesized according to the procedure of compound 16a with tert-butyl 9-formyl-3-azaspiro[5.5]undecane-3-carboxylate as start material. ¹H NMR (400 MHz, CD₃OD) δ ppm 0.88 (d, J=10.3 Hz, 2H), 0.95 (d, J=26.0 Hz, 2H), 1.10-1.30 (m, 4H), 1.42 (s, 2H), 1.67 (dd, J=29.7, 16.7 Hz, 5H), 1.76-1.95 (m, 2H), 2.13 (d, J=15.0 Hz, 10H), 2.22-2.34 (m, 1H), 2.35-2.51 (m, 1H), 2.63-2.82 (m, 2H), 2.95-3.20 (m, 8H), 3.33-3.47 (m, 2H), 3.66 (dd, J=30.7, 13.8 Hz, 4H), 3.90 (s, 1H), 4.02 (s, 4H), 4.26 (s, 2H), 4.36 (dd, J=8.8, 5.0 Hz, 1H), 4.39-4.55 (m, 2H), 7.06 (d, J=8.5 Hz, 1H), 7.18 (t, J=2.2 Hz, 1H), 7.35 (s, 3H), 7.69 (s, 1H), 7.88 (s, 1H), 9.04 (d, J=1.9 Hz, 1H). m/z (ESI): 1017.48 [M+H]⁺.

### Example 165. Synthesis of Compound 27a

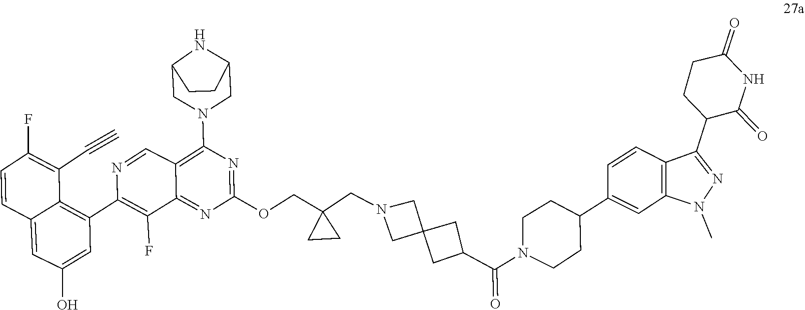 [SMILES: C#Cc1c(F)ccc2cc(O)cc(-c3ncc4c(N5CC6CCC(C5)N6)nc(OCC5(CN6CC7(CC(C(=O)N8CCC(c9ccc%10c(C%11CCC(=O)NC%11=O)nn(C)c%10c9)CC8)C7)C6)CC5)nc4c3F)c12]

Compound 27a was synthesized according to the procedure of compound 16a with 2-(tert-butoxycarbonyl)-2-azaspiro[3.3]heptane-6-carboxylic acid as start material. ¹H NMR (400 MHz, CD₃OD) δ 9.14 (s, 1H), 7.84 (tt, J=15.5, 7.5 Hz, 1H), 7.66 (t, J=8.7 Hz, 1H), 7.40-7.24 (m, 4H), 7.06 (dd, J=8.6, 4.8 Hz, 1H), 4.85 (s, 1H), 4.65 (d, J=13.2 Hz, 1H), 4.58-4.37 (m, 5H), 4.32 (d, J=9.5 Hz, 2H), 4.26-4.16 (m, 2H), 4.10-3.96 (m, 5H), 3.93-3.82 (m, 1H), 3.51-3.39 (m, 2H), 3.37 (s, 1H), 3.21-3.06 (m, 1H), 3.03-2.87 (m, 1H), 2.84-2.65 (m, 4H), 2.51 (dd, J=16.1, 6.5 Hz, 3H), 2.36 (dt, J=13.6, 5.8 Hz, 1H), 2.18 (q, J=11.7, 10.4 Hz, 4H), 1.90 (d, J=14.8 Hz, 2H), 1.63 (d, J=13.9 Hz, 2H), 1.32 (s, 2H), 0.91 (d, J=8.8 Hz, 4H). m/z (ESI): 975.27 [M+H]⁺.

### Example 166. Synthesis of Compound 28a

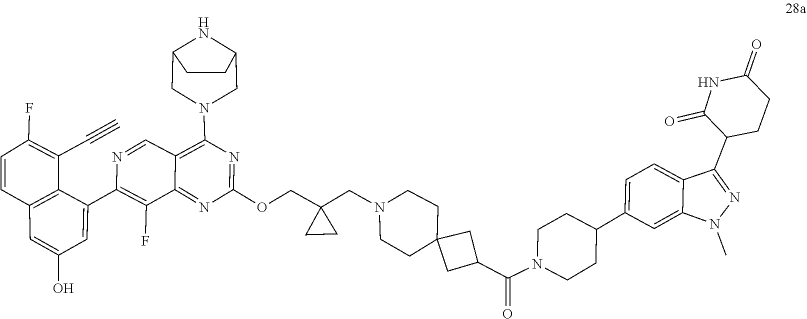 [SMILES: C#Cc1c(F)ccc2cc(O)cc(-c3ncc4c(N5CC6CCC(C5)N6)nc(OCC5(CN6CCC7(CC6)CC(C(=O)N6CCC(c8ccc9c(C%10CCC(=O)NC%10=O)nn(C)c9c8)CC6)C7)CC5)nc4c3F)c12]

Compound 28a was synthesized according to the procedure of compound 16a with 7-(tert-butoxycarbonyl)-7-azaspiro[3.5]nonane-2-carboxylic acid as start material. ¹H NMR (400 MHz, CD₃OD) δ 0.76 (s, 2H), 0.91 (s, 2H), 1.65-1.68 (m, 2H), 1.82-1.96 (m, 12H), 2.10-2.12 (m, 4H), 2.28-2.35 (m, 1H), 2.41-2.50 (m, 1H), 2.72-2.79 (m, 3H), 2.98 (t, J=12.4 Hz, 2H), 3.13-3.20 (m, 2H), 3.40-3.48 (m, 3H), 3.73-3.77 (m, 4H), 3.92 (d, J=13.6 Hz, 1H), 3.99 (s, 3H), 4.33-4.40 (m, 2H), 4.50-4.54 (m, 1H), 4.59-4.70 (m, 4H), 7.07 (d, J=8.4 Hz, 1H), 7.20 (s, 1H), 7.28-7.36 (m, 3H), 7.64-7.66 (m, 1H), 7.82-7.86 (m, 1H), 9.04 (s, 1H). m/z (ESI): 1003.22 [M+H]⁺.

### Example 167. Synthesis of Compound 29a

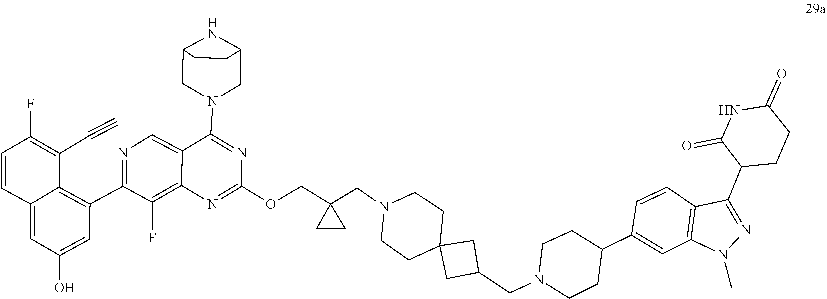 [SMILES: C#Cc1c(F)ccc2cc(O)cc(-c3ncc4c(N5CC6CCC(C5)N6)nc(OCC5(CN6CCC7(CC6)CC(CN6CCC(c8ccc9c(C%10CCC(=O)NC%10=O)nn(C)c9c8)CC6)C7)CC5)nc4c3F)c12]

Compound 29a was synthesized according to the procedure of compound 16a with tert-butyl 2-formyl-7-azaspiro[3.5]nonane-7-carboxylate as start material. ¹H NMR (400 MHz, CD₃OD) δ ppm 9.07 (s, 1H), 7.93-7.79 (m, 1H), 7.70 (d, J=8.3 Hz, 1H), 7.40-7.26 (m, 3H), 7.21 (s, 1H), 7.08 (d, J=8.3 Hz, 1H), 4.58-4.42 (m, 2H), 4.36 (dd, J=9.1, 5.1 Hz, 1H), 4.28 (d, J=10.7 Hz, 2H), 4.18-4.09 (m, 1H), 4.07-3.96 (m, 4H), 3.91 (d, J=14.1 Hz, 1H), 3.84-3.72 (m, 2H), 3.62-3.52 (m, 3H), 3.45 (d, J=22.1 Hz, 4H), 3.15-3.01 (m, 4H), 2.99-2.87 (m, 1H), 2.86-2.64 (m, 3H), 2.54-2.38 (m, 1H), 2.38-2.25 (m, 3H), 2.22-2.03 (m, 10H), 2.02-1.85 (m, 3H), 1.85-1.68 (m, 2H), 1.66-1.57 (m, 1H), 1.06-0.95 (m, 2H), 0.93-0.81 (m, 4H). m/z (ESI): 989.1 [M+H]⁺.

### Example 168. Synthesis of Compound 30a

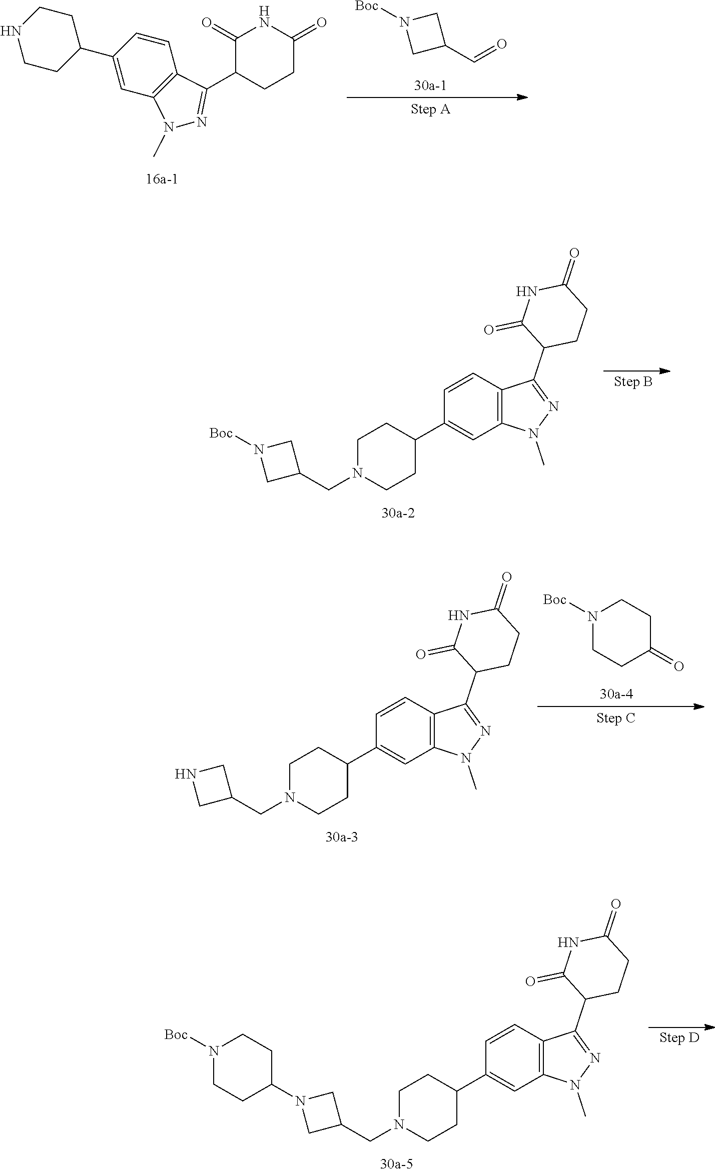 [SMILES: Cn1nc(C2CCC(=O)NC2=O)c2ccc(C3CCNCC3)cc21 ; CC(C)(C)OC(=O)N1CC(C=O)C1 ; Cn1nc(C2CCC(=O)NC2=O)c2ccc(C3CCN(CC4CN(C(=O)OC(C)(C)C)C4)CC3)cc21 ; Cn1nc(C2CCC(=O)NC2=O)c2ccc(C3CCN(CC4CNC4)CC3)cc21 ; CC(C)(C)OC(=O)N1CCC(=O)CC1 ; Cn1nc(C2CCC(=O)NC2=O)c2ccc(C3CCN(CC4CN(C5CCN(C(=O)OC(C)(C)C)CC5)C4)CC3)cc21]

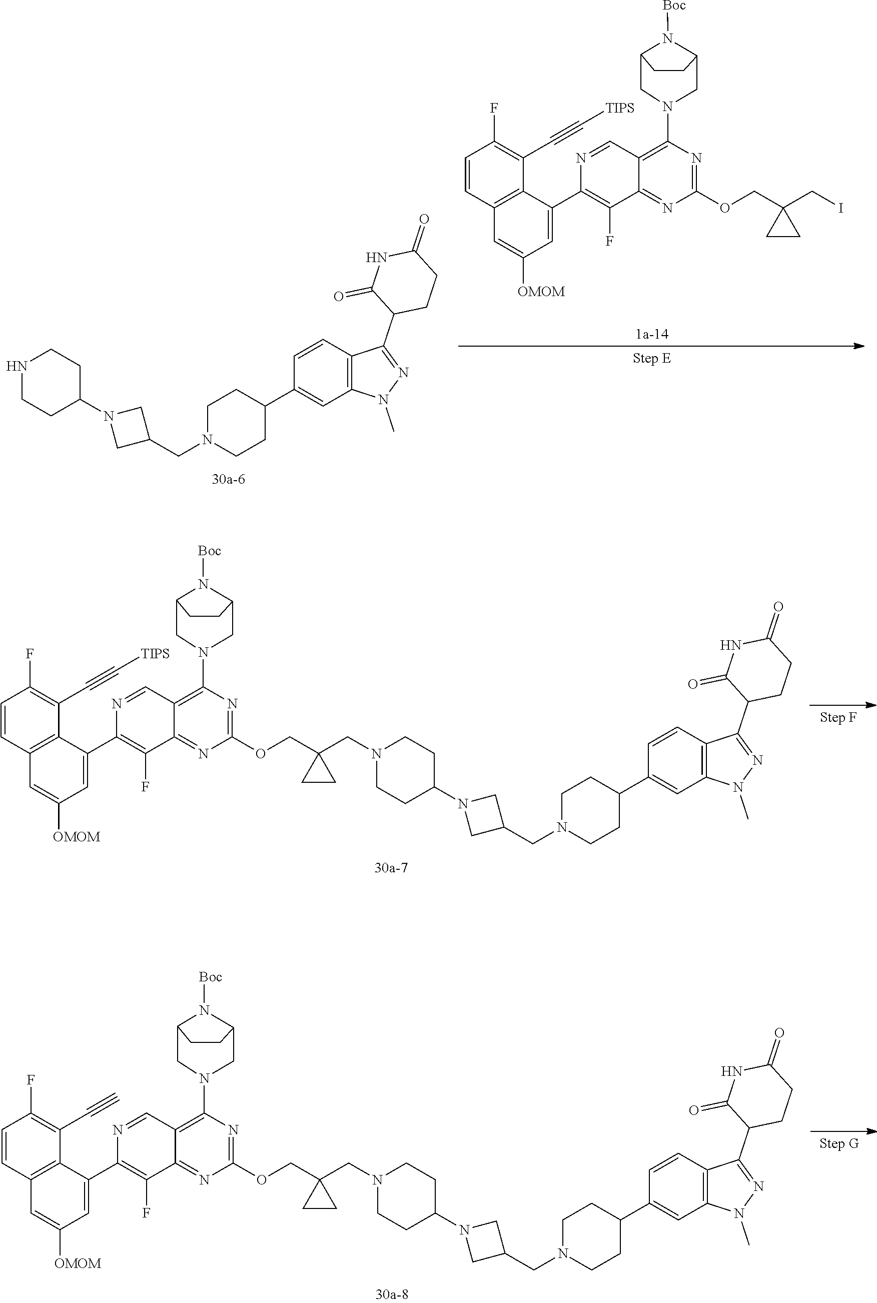

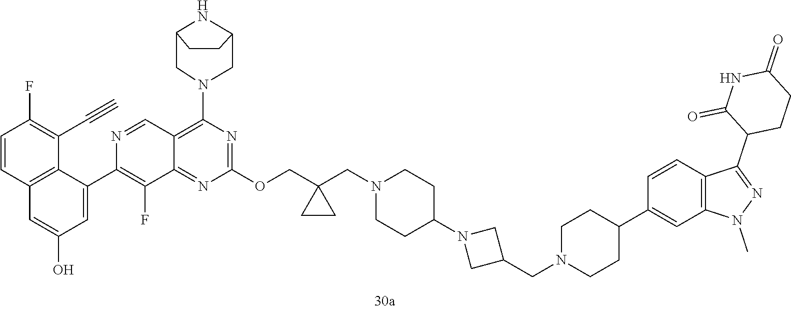 [SMILES: C#Cc1c(F)ccc2cc(O)cc(-c3ncc4c(N5CC6CCC(C5)N6)nc(OCC5(CN6CCC(N7CC(CN8CCC(c9ccc%10c(C%11CCC(=O)NC%11=O)nn(C)c%10c9)CC8)C7)CC6)CC5)nc4c3F)c12]

Step A: Et₃N (278.87 mg, 2.76 mmol, 5 eq) was added to a solution of compound 16a-1 (200 mg, 551.19 μmol, 1 eq) in MeOH (10 mL) and THF (10 mL), followed by addition of compound 30a-1 (122.51 mg, 661.43 μmol, 1.2 eq) and AcOH (165.49 mg, 2.76 mmol, 5 eq). The mixture was stirred at 50° C. for 2 h, followed by addition of NaBH₃CN (69.27 mg, 1.10 mmol, 2 eq). The reaction mixture was stirred at 50° C. for 2 h, then quenched with water, and extracted with DCM. The combined organic layer was washed with water and brine, then dried over Na₂SO₄ and filtered. The filtrate was concentrated under reduced pressure and the residue was purified by column chromatography (MeOH/DCM=0%-3%) to afford compound 30a-2 (270 mg, yield 98.8%).

Step B: 4M HCl in dioxane (2 mL) was added to a solution of compound 30a-2 (270 mg, 544.78 μmol, 1 eq) in DCM (5 mL). The reaction mixture was stirred at 25° C. for 30 min, then concentrated under reduced pressure to afford compound 30a-3 (260 mg, yield 99%).

Step C: Et₃N (216.02 mg, 2.13 mmol, 5 eq) was added to a solution of compound 30a-3 (200 mg, 426.97 μmol, 1 eq) in MeOH (5 mL) and THF (5 mL), followed by addition of compound 30a-4 (170.14 mg, 853.94 μmol, 2 eq) and AcOH (128.20 mg, 2.13 mmol, 5 eq). The mixture was stirred at 50° C. for 2 h, followed by addition of NaBH₃CN (53.66 mg, 853.94 μmol, 2 eq). The reaction mixture was stirred at 50° C. for 2 h, then quenched with water, and extracted with DCM. The combined organic layer was washed with water and brine, then dried over Na₂SO₄ and filtered. The filtrate was concentrated under reduced pressure and the residue was purified by column chromatography (MeOH/DCM=0%-7%) to afford compound 30a-5 (90 mg, yield 36.4%).

Step D: 4M HCl in dioxane (1.5 mL) was added to a solution of compound 30a-5 (90 mg, 155.51 μmol, 1 eq) in DCM (4 mL). The reaction mixture was stirred at 25° C. for 30 min, then concentrated under reduced pressure to afford compound 30a-6 (90 mg, yield 98.4%).

Step E: Compound 1a-14 (90 mg, 94.34 μmol, 1 eq) was added to a solution of compound 30a-6 (88.76 mg, 150.95 μmol, 1.6 eq) in DMF (5 mL), followed by addition of Et₃N (47.73 mg, 471.71 μmol, 5 eq). The mixture was stirred at 45° C. for 16 h, then cooled to 25° C. and concentrated under reduced pressure. The residue was purified by column chromatography (MeOH/DCM=0%-12%) to afford compound 30a-7 (55 mg, yield 44.7%).

Step F: 1M TBAF in THF (0.8 mL) was added to a solution of compound 30a-7 (20 mg, 94.00 μmol, 1 eq) in THF (0.6 mL). The mixture was stirred at 25° C. for 1 h, then quenched with water and extracted with EA. The combined organic layer was washed with water and brine, then dried over Na₂SO₄ and filtered. The filtrate was concentrated under reduced pressure and the residue was purified by column chromatography (MeOH/DCM=0%-15%) to afford compound 30a-8 (30 mg, yield 61.9%).

Step G: 2M HCl in EA (0.5 mL) was added to a solution of compound 30a-8 (30 mg, 26.12 μmol, 1 eq) in DCM (2 mL). The mixture was stirred at 25° C. for 30 min, then concentrated under reduced pressure. The residue was purified by pre-HPLC (0.1% TFA in water/acetonitrile) to afford compound 30a (3.6 mg, yield 8.3%). ¹H NMR (400 MHz, CD₃OD) δ ppm 9.09 (s, 1H), 7.88 (dd, J=9.1, 5.7 Hz, 1H), 7.70 (s, 1H), 7.41-7.30 (m, 3H), 7.22 (d, J=2.6 Hz, 1H), 7.10 (d, J=8.5 Hz, 1H), 4.51 (s, 2H), 4.43-4.20 (m, 5H), 4.19-3.89 (m, 8H), 3.68-3.47 (m, 7H), 3.42-3.34 (m, 4H), 3.20-2.98 (m, 5H), 2.86-2.64 (m, 2H), 2.53-2.41 (m, 1H), 2.39-2.25 (m, 3H), 2.24-2.06 (m, 9H), 2.06-1.91 (m, 2H), 1.06-0.98 (m, 2H), 0.94-0.82 (m, 2H). m/z (ESI): 1004.37 [M+H]⁺.

### Example 169. Synthesis of Compound 31a

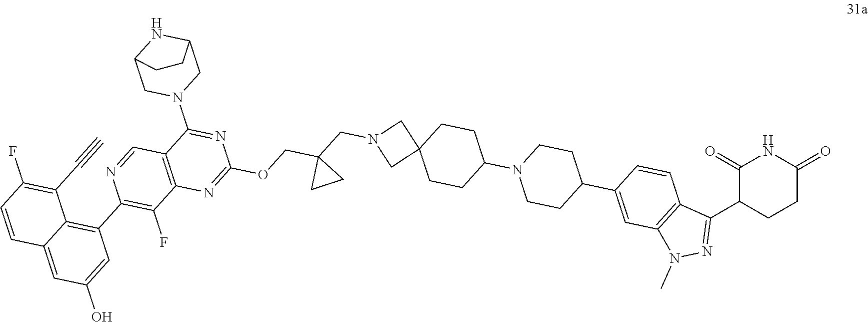 [SMILES: C#Cc1c(F)ccc2cc(O)cc(-c3ncc4c(N5CC6CCC(C5)N6)nc(OCC5(CN6CC7(CCC(N8CCC(c9ccc%10c(C%11CCC(=O)NC%11=O)nn(C)c%10c9)CC8)CC7)C6)CC5)nc4c3F)c12]

Compound 31a was synthesized according to the procedure of compound 16a with tert-butyl 7-oxo-2-azaspiro[3.5]nonane-2-carboxylate as start material. ¹H NMR (400 MHz, CD₃OD) δ 0.96 (s, 3H), 1.33 (d, J=8.9 Hz, 2H), 1.69 (td, J=21.1, 16.6, 10.3 Hz, 5H), 2.37 (d, J=11.4 Hz, 1H), 1.93-2.30 (m, 13H), 2.37 (d, J=11.4 Hz, 1H), 2.46-2.56 (m, 2H), 2.76-2.93 (m, 3H), 3.10-3.30 (m, 5H), 3.51 (d, J=10.4 Hz, 2H), 3.58 (t, J=17.7 Hz, 3H), 3.93 (d, J=9.1 Hz, 2H), 4.07 (s, 3H), 4.28-4.34 (m, 2H), 4.35-4.46 (m, 2H), 4.59 (dd, J=27.7, 11.7 Hz, 2H), 4.82 (t, J=8.1 Hz, 2H), 7.04 (dd, J=17.8, 8.4 Hz, 1H), 7.22-7.39 (m, 4H), 7.66-7.91 (m, 2H), 9.12 (d, J=7.2 Hz, 1H). m/z (ESI): 976.64 [M+H]⁺.

### Example 170. Synthesis of Compound 32a

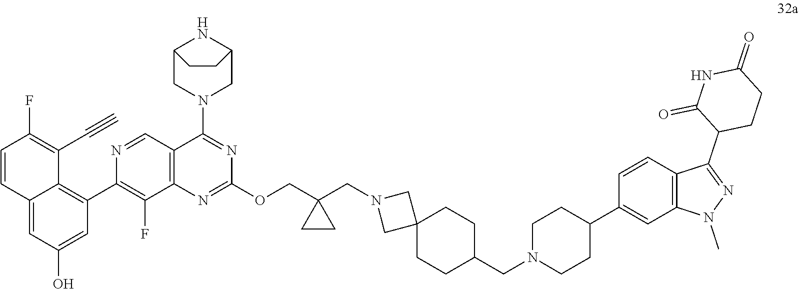 [SMILES: C#Cc1c(F)ccc2cc(O)cc(-c3ncc4c(N5CC6CCC(C5)N6)nc(OCC5(CN6CC7(CCC(CN8CCC(c9ccc%10c(C%11CCC(=O)NC%11=O)nn(C)c%10c9)CC8)CC7)C6)CC5)nc4c3F)c12]

Compound 32a was synthesized according to the procedure of compound 16a with tert-butyl 7-formyl-2-azaspiro[3.5]nonane-2-carboxylate as start material. ¹H NMR (400 MHz, CD₃OD) δ 1.62-1.74 (m, 8H), 1.78-2.01 (m, 5H), 2.05-2.29 (m, 13H), 2.30-2.43 (m, 3H), 2.78 (d, J=12.7 Hz, 3H), 3.03 (t, J=20.6 Hz, 5H), 3.66-3.78 (m, 2H), 3.87-4.00 (m, 4H), 4.06 (s, 3H), 4.32 (d, J=10.4 Hz, 3H), 4.45-4.56 (m, 2H), 7.12 (d, J=8.5 Hz, 1H), 7.26 (d, J=2.7 Hz, 1H), 7.38 (d, J=13.4 Hz, 3H), 7.74 (d, J=8.4 Hz, 1H), 7.84-7.96 (m, 1H), 9.12 (s, 1H). m/z (ESI): 990.29 [M+H]⁺.

### Example 171. Synthesis of Compound 33a

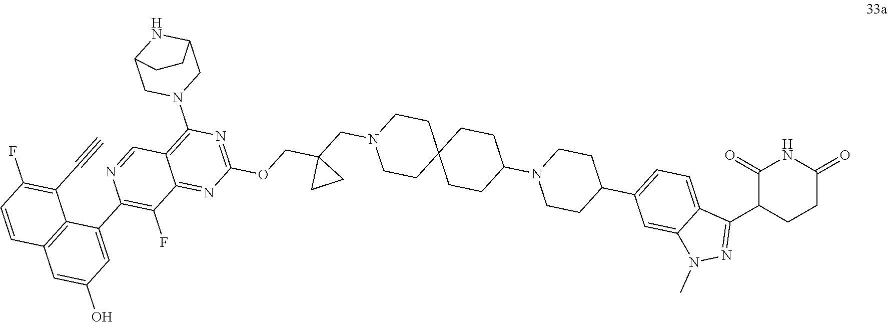 [SMILES: C#Cc1c(F)ccc2cc(O)cc(-c3ncc4c(N5CC6CCC(C5)N6)nc(OCC5(CN6CCC7(CCC(N8CCC(c9ccc%10c(C%11CCC(=O)NC%11=O)nn(C)c%10c9)CC8)CC7)CC6)CC5)nc4c3F)c12]

Compound 33a was synthesized according to the procedure of compound 16a with tert-butyl 9-oxo-3-azaspiro[5.5]undecane-3-carboxylate as start material. ¹H NMR (400 MHz, CD₃OD) δ 0.86-1.00 (m, 4H), 1.24-1.28 (m, 1H), 1.48-1.74 (m, 6H), 1.90-2.35 (m, 16H), 2.43-2.47 (m, 1H), 2.68-81 (m, 2H), 3.03-3.25 (m, 7H), 3.42-3.47 (m, 2H), 3.57-3.99 (m, 6H), 4.02 (s, 3H), 4.26-4.62 (m, 5H), 7.09 (d, J=8.4 Hz, 1H), 7.22 (s, 1H), 7.33-7.38 (m, 3H), 7.70 (d, J=8.8 Hz, 1H), 7.87-7.91 (m, 1H), 9.08 (s, 1H). m/z (ESI): 1003.32 [M+H]⁺.

### Example 172. Synthesis of Compound 34a

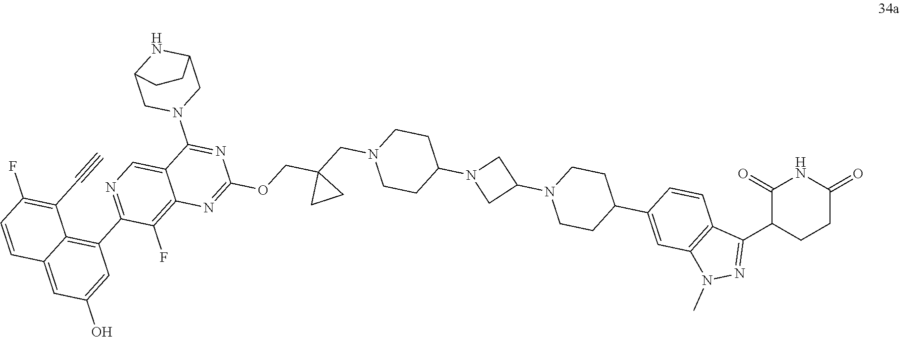 [SMILES: C#Cc1c(F)ccc2cc(O)cc(-c3ncc4c(N5CC6CCC(C5)N6)nc(OCC5(CN6CCC(N7CC(N8CCC(c9ccc%10c(C%11CCC(=O)NC%11=O)nn(C)c%10c9)CC8)C7)CC6)CC5)nc4c3F)c12]

Compound 34a was synthesized according to the procedure of compound 30a with tert-butyl 3-oxoazetidine-1-carboxylate as start material. ¹H NMR (400 MHz, CD₃OD) δ ppm 9.08 (s, 1H), 7.92-7.79 (m, 1H), 7.69 (d, J=8.5 Hz, 1H), 7.42-7.26 (m, 3H), 7.21 (d, J=2.6 Hz, 1H), 7.09 (d, J=8.4 Hz, 1H), 4.54-4.42 (m, 2H), 4.36 (dd, J=9.2, 5.1 Hz, 1H), 4.32-4.21 (m, 2H), 4.16-3.75 (m, 11H), 3.73-3.52 (m, 4H), 3.49-3.40 (m, 4H), 3.14-2.89 (m, 5H), 2.87-2.67 (m, 3H), 2.52-2.39 (m, 1H), 2.38-2.26 (m, 2H), 2.26-1.95 (m, 10H), 1.95-1.72 (m, 2H), 0.96-0.74 (m, 4H). m/z (ESI): 990.32 [M+H]⁺.

### Example 173. Synthesis of Compound 35a

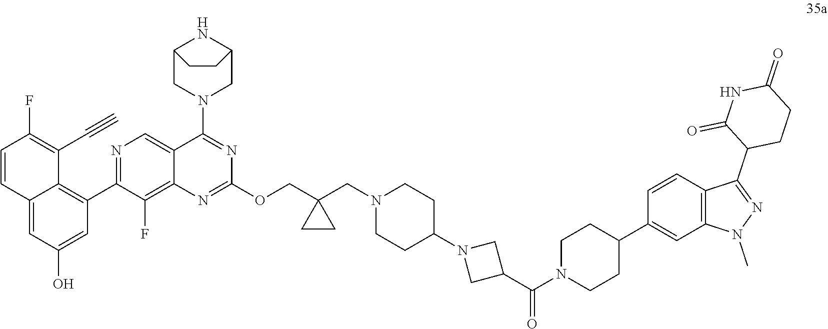 [SMILES: C#Cc1c(F)ccc2cc(O)cc(-c3ncc4c(N5CC6CCC(C5)N6)nc(OCC5(CN6CCC(N7CC(C(=O)N8CCC(c9ccc%10c(C%11CCC(=O)NC%11=O)nn(C)c%10c9)CC8)C7)CC6)CC5)nc4c3F)c12]

Compound 35a was synthesized according to the procedure of compound 30a with 1-(tert-butoxycarbonyl)azetidine-3-carboxylic acid as start material. ¹H NMR (400 MHz, CD₃OD) δ ppm 9.09 (s, 1H), 7.86 (q, J=6.9, 4.9 Hz, 1H), 7.65 (d, J=8.4 Hz, 1H), 7.41-7.27 (m, 3H), 7.22 (s, 1H), 7.08 (d, J=8.5 Hz, 1H), 4.83-4.76 (m, 3H), 4.68 (d, J=13.5 Hz, 1H), 4.58-4.24 (m, 10H), 4.22-3.86 (m, 9H), 3.75 (d, J=13.8 Hz, 1H), 3.64-3.51 (m, 1H), 3.50-3.35 (m, 3H), 3.25-2.93 (m, 5H), 2.88-2.67 (m, 3H), 2.53-2.27 (m, 4H), 2.24-2.10 (m, 4H), 2.07-1.90 (m, 4H), 1.82-1.60 (m, 2H), 1.06-0.98 (m, 2H), 0.94-0.84 (m, 2H). m/z (ESI): 1018.34 [M+H]⁺.

### Example 174. Synthesis of Compound 36a

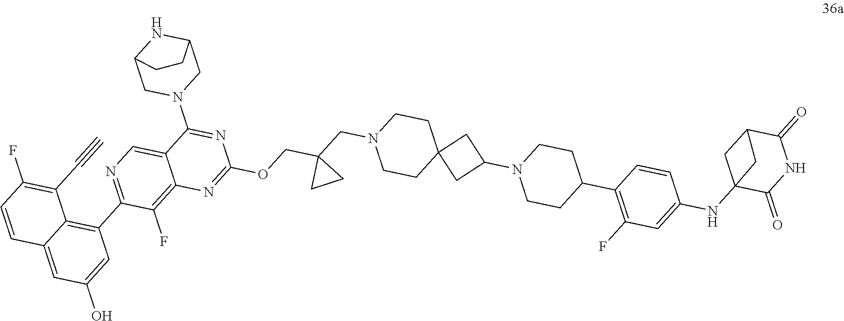 [SMILES: C#Cc1c(F)ccc2cc(O)cc(-c3ncc4c(N5CC6CCC(C5)N6)nc(OCC5(CN6CCC7(CC6)CC(N6CCC(c8ccc(NC9%10CC(C9)C(=O)NC%10=O)cc8F)CC6)C7)CC5)nc4c3F)c12]

Compound 36a was synthesized according to the procedure of compound 12a with intermediate g as start material. ¹H NMR (400 MHz, CD₃OD) δ ppm 9.08 (d, J=1.9 Hz, 1H), 7.88 (dd, J=9.3, 5.9 Hz, 1H), 7.41-7.30 (m, 2H), 7.22 (t, J=2.1 Hz, 1H), 6.98 (t, J=8.5 Hz, 1H), 6.36 (d, J=8.4 Hz, 1H), 6.28 (d, J=13.2 Hz, 1H), 4.60-4.42 (m, 2H), 4.28 (d, J=10.8 Hz, 2H), 4.06-3.81 (m, 3H), 3.79-3.64 (m, 2H), 3.56-3.47 (m, 2H), 3.47-3.36 (m, 3H), 3.28-3.17 (m, 2H), 3.16-2.95 (m, 5H), 2.94-2.74 (m, 4H), 2.70-2.60 (m, 2H), 2.58-2.46 (m, 1H), 2.38-1.82 (m, 16H), 1.06-0.95 (m, 2H), 0.95-0.83 (m, 2H). m/z (ESI): 966.33 [M+H]⁺.

### Example 175. Synthesis of Compound 37a

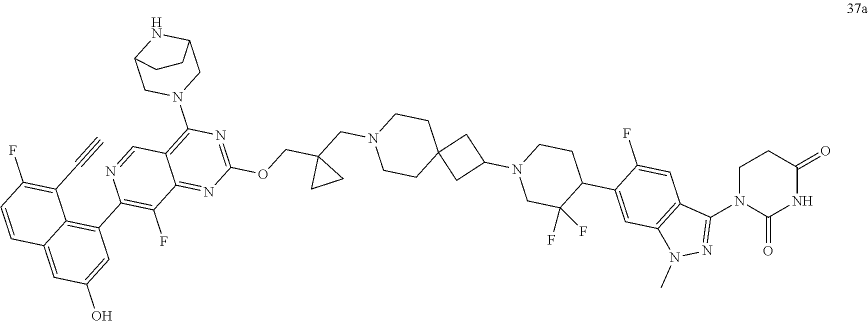 [SMILES: C#Cc1c(F)ccc2cc(O)cc(-c3ncc4c(N5CC6CCC(C5)N6)nc(OCC5(CN6CCC7(CC6)CC(N6CCC(c8cc9c(cc8F)c(N8CCC(=O)NC8=O)nn9C)C(F)(F)C6)C7)CC5)nc4c3F)c12]

Compound 37a was synthesized according to the procedure of compound 12a with intermediate j as start material. ¹H NMR (400 MHz, CD₃OD) δ 9.12 (s, 1H), 7.91 (dd, J=9.2, 5.6 Hz, 1H), 7.61 (d, J=5.3 Hz, 1H), 7.50 (d, J=10.2 Hz, 1H), 7.38 (dt, J=14.8, 4.1 Hz, 2H), 7.26 (d, J=2.5 Hz, 1H), 4.66-4.45 (m, 2H), 4.33 (d, J=10.8 Hz, 2H), 4.12-3.85 (m, 12H), 3.80 (s, 1H), 3.70 (d, J=11.0 Hz, 1H), 3.57-3.42 (m, 3H), 3.19-3.02 (m, 3H), 2.90 (t, J=6.7 Hz, 2H), 2.76-2.51 (m, 2H), 2.34 (d, J=21.1 Hz, 3H), 2.22-2.16 (m, 4H), 2.06 (d, J=34.0 Hz, 4H), 1.34 (dd, J=11.3, 5.5 Hz, 3H), 1.05 (d, J=9.0 Hz, 2H), 0.92 (d, J=6.6 Hz, 2H). ¹⁹F NMR (376 MHz, CD₃OD) δ −77.08, −104.84 (d, J=258.0 Hz), −110.98 (d, J=258.6 Hz), −111.57 (d, J=12.6 Hz), −127.17, −138.32-−139.08 (m). m/z (ESI): 1030.32 [M+H]⁺.

### Example 176. Synthesis of Compound 38a

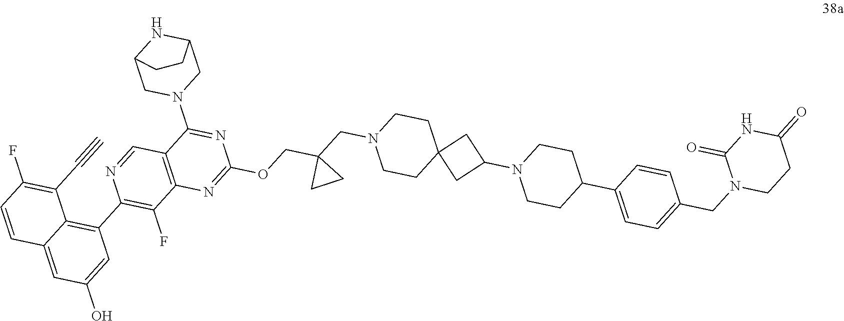 [SMILES: C#Cc1c(F)ccc2cc(O)cc(-c3ncc4c(N5CC6CCC(C5)N6)nc(OCC5(CN6CCC7(CC6)CC(N6CCC(c8ccc(CN9CCC(=O)NC9=O)cc8)CC6)C7)CC5)nc4c3F)c12]

Compound 38a was synthesized according to the procedure of compound 1a with (4-bromophenyl)methanamine as start material. ¹H NMR (400 MHz, CD₃OD) δ 0.92 (d, J=7.9 Hz, 2H), 0.98-1.10 (m, 3H), 1.94-2.35 (m, 16H), 2.65 (t, J=6.8 Hz, 2H), 2.93 (d, J=11.3 Hz, 3H), 3.02-3.15 (m, 2H), 3.40 (t, J=6.9 Hz, 3H), 3.60 (d, J=11.8 Hz, 2H), 3.76 (dq, J=15.9, 8.0, 7.0 Hz, 2H), 3.92 (dd, J=24.6, 13.3 Hz, 2H), 4.03 (d, J=13.9 Hz, 1H), 4.32 (d, J=10.8 Hz, 2H), 4.50 (t, J=10.5 Hz, 1H), 4.60 (d, J=14.5 Hz, 3H), 4.79 (dd, J=17.6, 3.5 Hz, 1H), 7.20-7.46 (m, 7H), 7.91 (dd, J=9.2, 5.7 Hz, 1H), 9.12 (s, 1H). ¹⁹F NMR (376 MHz, CD₃OD) δ −138.72 (s, 1F), 111.61 (d, J=11.7 Hz, 1F), −77.07 (s, 15F). m/z (ESI): 966.33 [M+H]⁺.

### Example 177. Synthesis of Compound 39a

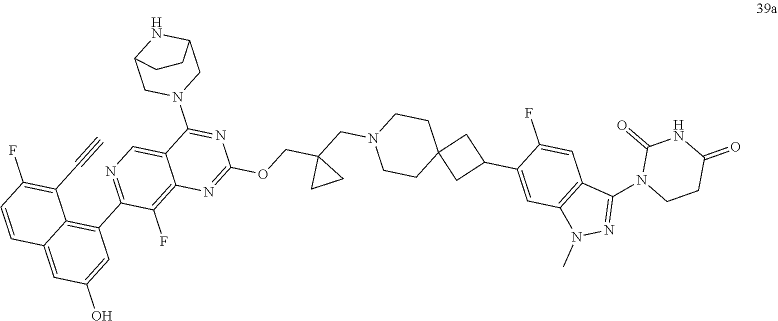 [SMILES: C#Cc1c(F)ccc2cc(O)cc(-c3ncc4c(N5CC6CCC(C5)N6)nc(OCC5(CN6CCC7(CC6)CC(c6cc8c(cc6F)c(N6CCC(=O)NC6=O)nn8C)C7)CC5)nc4c3F)c12]

Compound 39a was synthesized according to the procedure of compound 2a with intermediate h as start material. ¹H NMR (400 MHz, CD₃OD) δ ppm 9.11 (s, 1H), 7.90-7.75 (m, 1H), 7.39-7.20 (m, 5H), 4.63 (t, J=11.8 Hz, 1H), 4.46 (t, J=10.6 Hz, 1H), 4.35-4.23 (m, 2H), 4.12-3.89 (m, 8H), 3.87-3.68 (m, 3H), 3.40 (d, J=14.0 Hz, 2H), 3.26 (d, J=13.7 Hz, 1H), 3.21-2.95 (m, 2H), 2.88 (t, J=6.8 Hz, 2H), 2.63-2.48 (m, 1H), 2.42-1.85 (m, 12H), 1.08-0.98 (m, 2H), 0.96-0.84 (m, 2H); ¹⁹F NMR (376 MHz, CD₃OD) δ −77.12 (s, 12F), −111.47 (dt, J=29.5, 7.0 Hz, 1F), −127.27-−127.60 (m, 1F), −138.38 (d, J=14.3 Hz, 1F). m/z (ESI): 911.59 [M+H]⁺.

### Example 178. Synthesis of Compound 40a

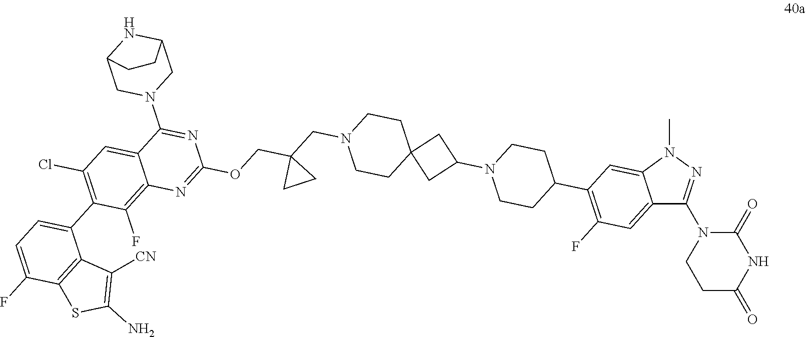 [SMILES: Cn1nc(N2CCC(=O)NC2=O)c2cc(F)c(C3CCN(C4CC5(CCN(CC6(COc7nc(N8CC9CCC(C8)N9)c8cc(Cl)c(-c9ccc(F)c%10sc(N)c(C#N)c9%10)c(F)c8n7)CC6)CC5)C4)CC3)cc21]

Compound 40a was synthesized according to the procedure of compound 2a with intermediate f as start material. ¹H NMR (400 MHz, CD₃OD) δ 7.96 (d, J=1.5 Hz, 1H), 7.51-7.39 (m, 2H), 7.25 (dd, J=8.4, 5.0 Hz, 1H), 7.09 (t, J=8.9 Hz, 1H), 4.68 (t, J=17.6 Hz, 2H), 4.56 (d, J=11.9 Hz, 1H), 4.44 (d, J=11.5 Hz, 1H), 4.28 (s, 2H), 4.11-4.04 (m, 5H), 3.96-3.76 (m, 5H), 3.65 (t, J=10.0 Hz, 2H), 3.19-2.98 (m, 4H), 2.90 (t, J=6.7 Hz, 2H), 2.58 (s, 1H), 2.41-1.95 (m, 16H), 1.03 (s, 2H), 0.91 (s, 2H). ¹⁹F NMR (376 MHz, CD₃OD) δ −77.03, −118.29, −122.77 (d, J=8.9 Hz), −128.90-−129.18 (m). m/z (ESI): 1033.50 [M+H]⁺.

### Example 179. Synthesis of Compound 41a

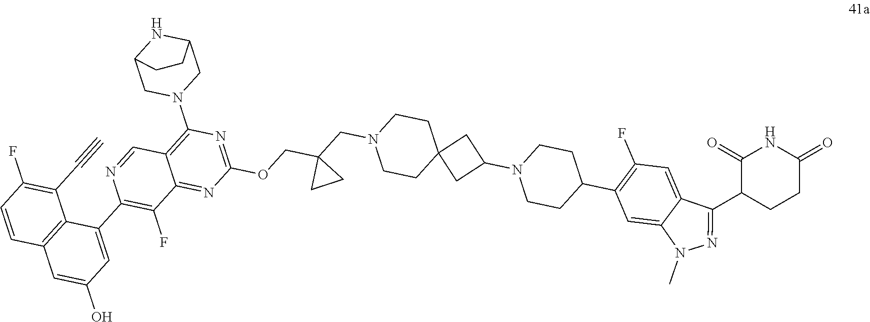 [SMILES: C#Cc1c(F)ccc2cc(O)cc(-c3ncc4c(N5CC6CCC(C5)N6)nc(OCC5(CN6CCC7(CC6)CC(N6CCC(c8cc9c(cc8F)c(C8CCC(=O)NC8=O)nn9C)CC6)C7)CC5)nc4c3F)c12]

Compound 41a was synthesized according to the procedure of compound 12a with intermediate i as start material. ¹H NMR (400 MHz, CD₃OD) δ 0.52 (s, 2H), 0.72 (s, 2H), 1.59-1.68 (m, 6H), 1.79-2.03 (m, 12H), 2.26-2.36 (m, 1H), 2.41-2.48 (m, 3H), 2.67-2.83 (m, 3H), 2.93-2.99 (m, 1H), 3.05-3.08 (m, 2H), 3.66-3.73 (m, 4H), 4.02 (s, 3H), 4.29-4.34 (m, 1H), 4.44-4.47 (m, 1H), 4.55-4.65 (m, 9H), 7.20 (d, J=2.8 Hz, 1H), 7.29-7.41 (m, 4H), 7.83-7.86 (m, 1H), 8.99 (s, 1H). m/z (ESI): 993.31 [M+H]⁺.

### Biological Assays

### Example 180. Protein Degradation Study

Aspc-1 (Cobioer, CBP60546) cells in exponential growth phase were inoculated on a 6-well cell culture plate (Corning, 3516) at density of 1E6/well; and cells in the plate were cultured in 37° C. incubator containing 5%-carbon dioxide. The next day, testing compounds were dissolved in DMSO (Sigma, RNBF5902) for the preparation of a 10-mM concentration stock solution. The stock solution was diluted with complete medium (RPMI-1640 supplemented with 10% fetal bovine serum, 1% penicillin-streptomycin), yielding working solutions of various concentrations. The plate, after the addition of compounds, was placed in a 5% carbon dioxide incubator, and incubated at 37° C. for 24 hours (final DMSO concentration was 0.1%). The plate was then removed from the incubator, and the cells were washed twice with precooled PBS (Gibco, 14190250), followed by addition of 80-μL RIPA lysis buffer (containing protease inhibitor (Invitrogen™, AM2696)) to each well. The adherent cells were scraped off with a cell scraper, and the cell lysate was transferred to a 1.5-mL centrifuge tube followed with a 30-minute incubation on ice. The lysate was then clarified with centrifugation at 14000 rpm for 10 min at 4° C. The supernatant was transferred into a new 1.5-mL centrifuge tube, and protein concentration was measured with BCA protein detection kit (Thermo Fisher, 23225). G12D protein level was assessed by western blot.

A mixture of cell lysate (40 μL) and loading buffer (10 μL, 5×SDS (Beyotime, P0015L)) was denatured in a water-bath at 95° C. for 10 min. The denatured protein sample, at 30 μL/well, was loaded into corresponding well of 4-20% Bis-Tris gel (Kingsley, M00656). Protein samples were initially electrophoresed at 80 V for 30 minutes, followed by 120 V for 40 minutes, until the bromophenol blue strip ran to an appropriate position. After electrophoresis, iBlot2 (Life Technologies, IB21001) was used to transfer the separated samples onto PVDF film. The PVDF membrane was washed with double-distilled water and then blocked in TBST (Thermo Scientific, 28360) buffer containing 5% non-fat milk, for 2 hours. Blocked membrane was rinsed with tris-buffered saline tween-20 (Thermo Scientific, 28360, TBST) three times, 10 minutes each. Membrane was then incubated with 1:1000 diluted KRAS primary antibody (CST, cat #14429S) overnight at 4° C. Membrane was rinsed in a way described previously followed by 1 hour incubation of secondary antibody at room temperature. After washing the film for three times with TBST, ECL color developing solution was applied, and image was captured with Biorad Chemi Doc gel imager. The band gray value was quantified (Image Lab), and protein degradation level was calculated according to the following formula:

Level of RAS protein expression=(RAS-compound/GAPDH)/(RAS-DMSO/GAPDH)

Level of degradation (%)=(1−Rate of RAS protein expression)×100

The degradation level of Ras protein in the presence of testing compounds in Aspc-1 cells were summarized in Tables 3-5. Symbols of −, +, ++, +++ indicate that the degradation level of Ras protein induced by testing compounds are 1000 or less, 11 to 30%, 31 to 60% and greater than 60%, respectively.

TABLE 4
KRAS^{G12D} protein degradation in Aspc-1
cells treated with 1 μM exemplary compounds

| Compd | Rate of protein |
|---|---|
| ID | degradation |
| 28 | + |
| 29 | ++ |
| 30 | + |
| 31 | +++ |
| 32 | + |
| 33 | +++ |
| 35 | ++ |
| 41 | +++ |
| 43 | +++ |
| 44 | ++ |
| 45 | +++ |
| 46 | +++ |
| 47 | ++ |
| 50 | + |
| 53 | + |
| 54 | − |
| 55 | +++ |
| 56 | +++ |
| 57 | +++ |
| 58 | ++ |
| 59 | − |
| 60 | + |
| 61 | − |
| 62 | − |
| 63 | + |
| 64 | + |
| 65 | + |
| 67 | ++ |
| 68 | +++ |
| 69 | +++ |
| 70 | +++ |
| 72 | +++ |

TABLE 5
RAS^{G12D} protein degradation in Aspc-1 cells
treated with 0.1 μM exemplary compounds

| Compd | Rate of protein |
|---|---|
| ID | degradation |
| 33 | ++ |
| 45 | ++ |
| 55 | +++ |
| 56 | ++ |
| 57 | +++ |
| 58 | + |
| 59 | − |
| 60 | − |
| 61 | − |
| 62 | − |
| 63 | − |
| 64 | − |
| 65 | − |
| 67 | − |
| 68 | ++ |
| 69 | +++ |
| 70 | + |
| 71 | − |
| 72 | +++ |
| 73 | − |
| 74 | ++ |
| 75 | +++ |
| 76 | − |
| 77 | − |
| 78 | − |
| 80 | ++ |
| 81 | ++ |
| 82 | +++ |
| 83 | + |
| 84 | ++ |
| 85 | +++ |
| 86 | ++ |
| 87 | ++ |
| 88 | − |
| 89 | − |
| 90 | − |
| 91 | − |
| 92 | +++ |
| 93 | +++ |
| 94 | ++ |
| 95 | + |
| 96 | + |
| 97 | ++ |
| 98 | ++ |
| 99 | +++ |
| 100 | +++ |
| 101 | + |
| 103 | − |
| 104 | − |
| 105 | ++ |
| 106 | + |
| 107 | + |
| 108 | − |
| 109 | ++ |
| 110 | ++ |
| 111 | +++ |
| 112 | +++ |
| 113 | +++ |
| 114 | +++ |
| 115 | ++ |
| 116 | ++ |
| 117 | +++ |
| 118 | +++ |
| 119 | + |
| 120 | + |
| 131 | − |
| 132 | + |
| 141 | +++ |

TABLE 6
RAS^{G12D} protein degradation in Aspc-1
cells treated with 50 nM exemplary compounds

| Compd ID | Rate of degradation | Compd ID | Rate of degradation |
|---|---|---|---|
| 124 | + | 125 | ++ |
| 126 | − | 127 | +++ |
| 128 | +++ | 129 | +++ |
| 133 | +++ | 134 | + |
| 135 | ++ | 136 | − |
| 137 | − | 138 | +++ |
| 139 | ++ | 140 | ++ |
| 141 | + | 142 | ++ |
| 143 | + | 144 | ++ |
| 145 | − | 146 | +++ |
| 147 | ++ | 148 | − |
| 149 | +++ | 150 | +++ |
| 151 | +++ | 152 | ++ |
| 153 | ++ | 154 | +++ |
| 155 | +++ | 156 | ++ |
| 157 | + | 158 | +++ |
| 159 | ++ | 160 | +++ |
| 161 | ++ | 162 | − |
| 163 | ++ | 164 | +++ |
| 165 | ++ | 166 | +++ |
| 167 | +++ | 168 | +++ |
| 169 | +++ | 170 | ++ |
| 171 | − | | |
| 1a | +++ | 2a | +++ |
| 4a | − | 11a | − |
| 12a | +++ | 13a | +++ |
| 14a | +++ | 15a | − |
| 16a | +++ | 17a | +++ |
| 18a | − | 19a | + |
| 20a | − | 21a | +++ |
| 22a | ++ | 23a | +++ |
| 24a | ++ | 25a | ++ |
| 26a | + | 27a | + |
| 28a | +++ | 29a | + |
| 30a | +++ | 31a | +++ |
| 32a | ++ | 33a | +++ |
| 34a | +++ | 35a | +++ |
| 36a | +++ | 41a | +++ |

TABLE 7
RAS^{G12D} protein degradation in Aspc-1 cells
treated with 0.005 μM exemplary compounds

| Compd | Rate of | Compd | Rate of | Compd | Rate of |
|---|---|---|---|---|---|
| ID | degradation | ID | degradation | ID | degradation |
| 1a | +++ | 2a | +++ | 4a | − |
| 7a | − | 10a | − | 11a | − |
| 12a | +++ | 13a | +++ | 14a | +++ |
| 15a | − | 16a | +++ | 17a | +++ |
| 18a | + | 19a | + | 20a | − |
| 21a | ++ | 22a | + | 23a | +++ |
| 24a | +++ | 25a | ++ | 26a | +++ |
| 27a | + | 28a | +++ | 29a | +++ |
| 30a | +++ | 31a | +++ | 32a | +++ |
| 33a | +++ | 34a | ++ | 35a | ++ |
| 36a | +++ | 38a | + | 39a | +++ |
| 40a | +++ | 41a | +++ | | |

TABLE 8
RAS^{G12D} protein degradation in Aspc-1
cells treated with 1 nM exemplary compounds

| Compd ID | Rate of degradation | Compd ID | Rate of degradation |
|---|---|---|---|
| 7a | + | 8a | + |
| 12a | +++ | 13a | +++ |
| 14a | +++ | | |

Half-maximum degradation concentrations (DC50s) of testing compounds were calculated using GraphPad Prism and their values were shown in Table 6. Symbols of ++++, ++++ and + represent DC50s equal or less than 10 nM, 11-50 nM, 51-100 nM, and 101-200 nM, respectively.

TABLE 9
DC50 of compounds induced Ras G12D
protein degradation inAspc-1 cells

| Compd ID | DC₅₀ (nM) | Compd ID | DC₅₀ (nM) |
|---|---|---|---|
| 33 | +++ | 43 | ++ |
| 45 | +++ | 55 | ++ |
| 56 | +++ | 57 | ++++ |
| 75 | ++++ | 82 | ++++ |
| 83 | + | 92 | ++++ |
| 93 | ++ | 99 | +++ |
| 110 | ++++ | 111 | ++++ |
| 112 | ++++ | 113 | ++++ |
| 114 | ++++ | 115 | +++ |
| 118 | ++++ | 129 | ++++ |
| 133 | ++++ | 135 | ++++ |
| 146 | ++++ | 149 | ++++ |
| 150 | ++++ | 151 | ++++ |
| 154 | ++++ | 155 | +++ |
| 156 | +++++ | 157 | − |
| 158 | ++++ | 160 | ++++ |
| 164 | ++++ | | |
| 1a | ++++ | 2a | ++++ |
| 12a | ++++ | 13a | ++++ |
| 14a | ++++ | 17a | ++++ |
| 21a | ++++ | 23a | ++++ |
| 24a | ++++ | 26a | ++++ |
| 28a | ++++ | 29a | ++++ |
| 30a | ++++ | 31a | ++++ |
| 32a | ++++ | 33a | ++++ |
| 36a | ++++ | 37a | ++++ |
| 39a | ++++ | 40a | ++++ |

The results indicate KRAS-G12D protein degradation in AsPC-1 cells in the presence of test compounds.

### Example 181. Cell Proliferation Study

Aspc-1 (Cobioer, CBP60546) cells were grown in RPMI 1640 (Gibco, 61870127) supplemented with 10% fetal bovine serum (Gibco, 10099141), 1% penicillin-streptomycin (Gibco, 15070-063). Aspc-1 cells in exponential growth phase were seeded into 96-well plate (Corning, 3599) at density of 4×10³/well. Cells were cultured overnight at 37° C. with 5% CO₂. On the next day, cells were treated with compound at various concentrations for 72 hours in 37° C., 5% CO₂ incubator (final DMSO concentration is 0.1%). After treatment, the cell plates were added 100 μL Cell Tier Glo (Promega, G7573) to every well and incubated at RT for 10 minutes. Cell growth status was measured with Cell Tier Glo (Promega, G7573) following manufacturer's directions. The inhibition curve was obtained with GraphPad 7.0 software using four-parameter equation.

Table 7 shows the inhibitory effect of exemplary compounds in Aspc-1 cells, where IC₅₀ values of the compounds equal or less than 100 nM, 101 nM-1 uM, 1-5 uM and 5-10 uM were categorized into ++++, +++, ++ and +, respectively.

TABLE 7
Inhibitory effects of test compounds on
cellular proliferation in AsPC-1 cells.

| Compd ID | IC₅₀ |
|---|---|
| 26 | ++ |
| 27 | +++ |
| 29 | ++ |
| 31 | ++ |
| 32 | ++ |
| 33 | +++ |
| 35 | ++ |
| 39 | + |
| 41 | +++ |
| 42 | ++ |
| 43 | +++ |
| 44 | ++ |
| 45 | +++ |
| 46 | +++ |
| 47 | ++ |
| 48 | ++ |
| 49 | ++ |
| 50 | ++ |
| 82 | ++ |
| 92 | ++++ |
| 111 | ++++ |
| 112 | ++++ |
| 129 | ++++ |
| 133 | ++++ |
| 135 | ++++ |
| 146 | ++++ |
| 149 | ++++ |
| 150 | ++++ |
| 151 | ++++ |
| 154 | ++++ |
| 155 | ++++ |
| 156 | ++++ |
| 157 | +++ |
| 158 | ++++ |
| 160 | ++++ |
| 164 | ++++ |
| 168 | ++++ |
| 169 | ++++ |
| 1a | ++++ |
| 2a | ++++ |
| 12a | ++++ |
| 13a | ++++ |
| 14a | +++ |
| 16a | +++ |
| 17a | ++++ |
| 21a | ++++ |
| 23a | ++++ |
| 24a | ++++ |
| 26a | ++++ |
| 28a | ++++ |
| 29a | ++++ |
| 30a | ++++ |
| 31a | ++++ |
| 32a | ++++ |
| 33a | ++++ |
| 37a | ++++ |
| 40a | ++++ |

The results indicate inhibitory effects of test compounds on cellular proliferation in AsPC-1 cells.

The contents of all documents and references cited herein are hereby incorporated by reference in their entirety.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

What is claimed is:

1. A compound of Formula (I), or a pharmaceutically acceptable salt, ester, hydrate, solvate, or stereoisomer thereof:
W-L-T (I)
where:
W is a targeting group that binds specifically to KRAS-G12D protein;
T is an E3-ligase binding group; and
L is absent or is a bivalent linking group that connects W and T together via a covalent linkage;
wherein the E3-ligase binding group T has the structure of Formula (IIIa) or Formula (IIIb): 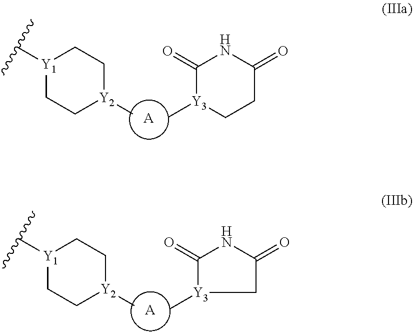
wherein:
A has the structure of 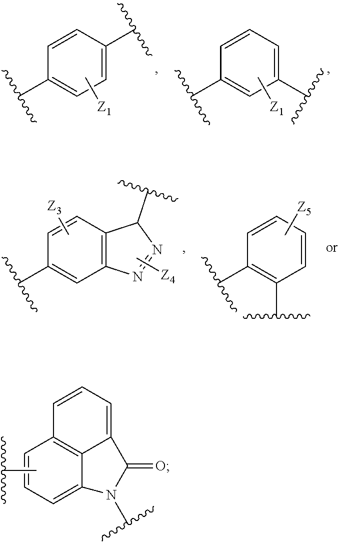
Z₁, Z₂ and Z₅ are independently substituted or unsubstituted C₁-C₂₀ alkyl, halogen (X), oxygen (O) or absent;
Z₃ and Z₄ are independently hydrogen (H), halogen (X), alkyl or absent;
Z₁, Z₂, Z₃, Z₄ and Z₅ can be substituted at any site on the benzene ring or the five-membered ring in A; and
Y₁, Y₂ and Y₃ are independently carbon (C) or nitrogen (N).

2. The compound of claim 1, wherein W has the structure of Formula (Ia) or (Ib): 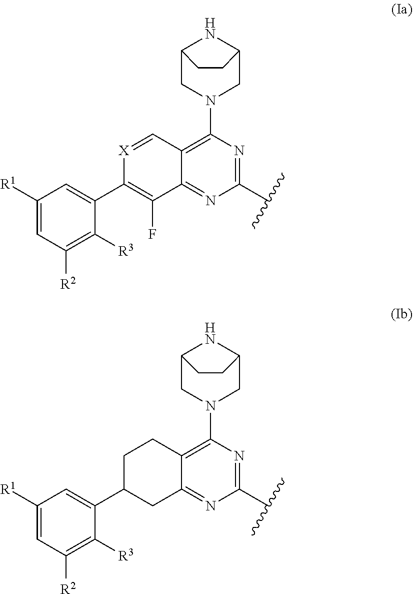
where:
X is a nitrogen (N) or an unsubstituted or substituted carbon (C);
R¹ is an unsubstituted or substituted hydroxyl, amino, or thio group; and
R² and R³ are independently hydrogen (H), halogen (X), or halogen substituted methyl, or R² and R³, together with the phenyl-ring structure to which they are attached, form an unsubstituted or substituted benzo-fused ring.

3. The compound of claim 2, wherein:
the substituted carbon is CH, C—F, C—Cl, C—CH₃, C—C₂H₅, or C—C₃H₇;
the halogen substituted methyl is —CH₂X, —CHX₂, or —CX₃; and/or
the benzo-fused ring is a naphthyl ring system, optionally substituted with one or more substituents selected from halogen, hydroxyl, amino, halomethyl, C₁-C₂ alkyl, and C₂ to C₄ alkynyl group.

4.-7. (canceled)

8. The compound of claim 2, wherein W has the structure of Formula (Ia); X is N; R¹ is —OH; and/or R² and R³, together with the phenyl-ring structure to which they are attached, form a substituted benzo-fused ring;
optionally wherein the targeting group W comprises a fragment having the structure: 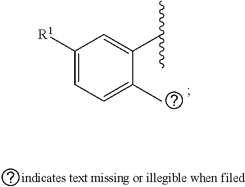
optionally wherein the fragment is:

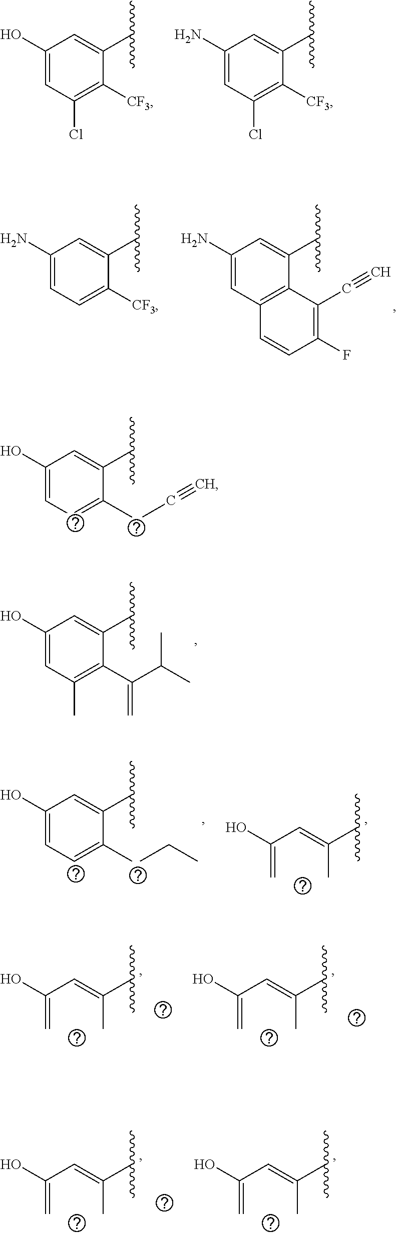

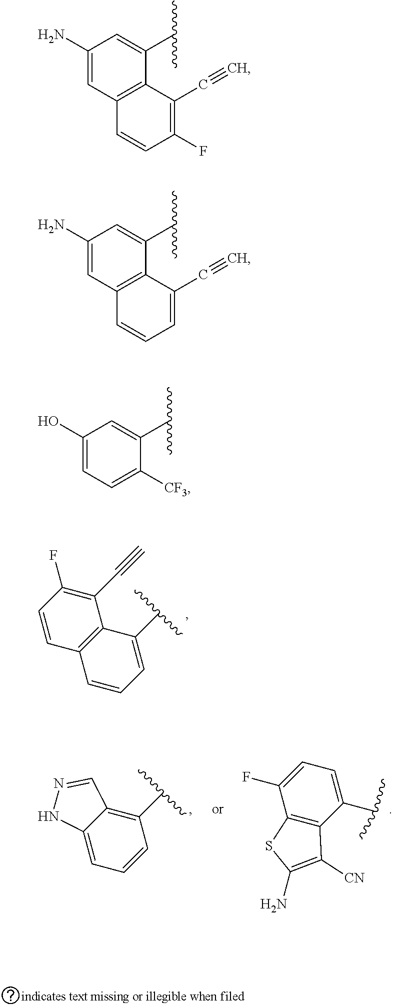 [SMILES: *c1cc(N)cc2ccc(F)c(C#C)c12 ; *c1cc(N)cc2cccc(C#C)c12 ; *c1cc(O)ccc1C(F)(F)F ; *c1cccc2ccc(F)c(C#C)c12 ; *c1cccc2[nH]ncc12 ; *c1ccc(F)c2sc(N)c(C#N)c12]

9.-15. (canceled)

16. The compound of claim 1, wherein:
Y₁ is N; Y₃ is N; or both Y₁ and Y₃ are N;
Y₂ is C;
Z₁, Z₂ and Z₅ are independently substituted or unsubstituted C₁-C₆ alkyl, halogen or absent; and/or
Z₃ and Z₄ are independently substituted or unsubstituted C₁-C₆ alkyl, halogen or absent;
optionally wherein the substituted or unsubstituted C₁-C₆ alkyl is CH₃;
optionally wherein the halogen is fluorine (F), chlorine (Cl) or bromine (Br), preferably fluorine (F).

17.-25. (canceled)

26. The compound of claim 16, wherein Z₁, Z₂ and Z₅ are independently CH₃, F or absent; and/or, wherein Z₃ and Z₄ are independently CH₃, F or absent.

27. (canceled)

28. The compound of claim 1, wherein the E3-ligase binding group T is: 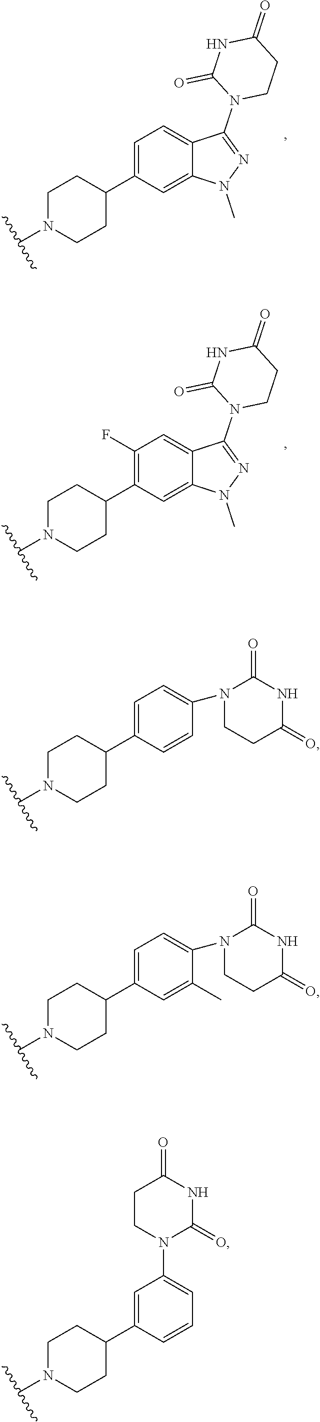 [SMILES: *N1CCC(c2ccc3c(N4CCC(=O)NC4=O)nn(C)c3c2)CC1 ; *N1CCC(c2cc3c(cc2F)c(N2CCC(=O)NC2=O)nn3C)CC1 ; *N1CCC(c2ccc(N3CCC(=O)NC3=O)cc2)CC1 ; *N1CCC(c2ccc(N3CCC(=O)NC3=O)c(C)c2)CC1 ; *N1CCC(c2cccc(N3CCC(=O)NC3=O)c2)CC1] 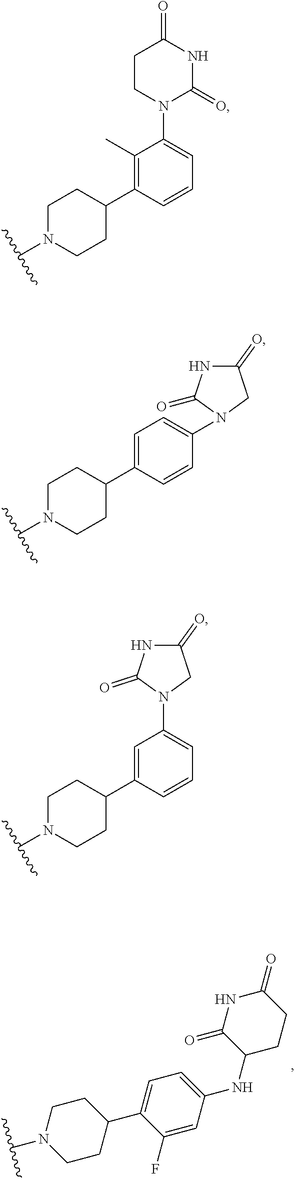 [SMILES: *N1CCC(c2cccc(N3CCC(=O)NC3=O)c2C)CC1 ; *N1CCC(c2ccc(N3CC(=O)NC3=O)cc2)CC1 ; *N1CCC(c2cccc(N3CC(=O)NC3=O)c2)CC1 ; *N1CCC(c2ccc(NC3CCC(=O)NC3=O)cc2F)CC1] 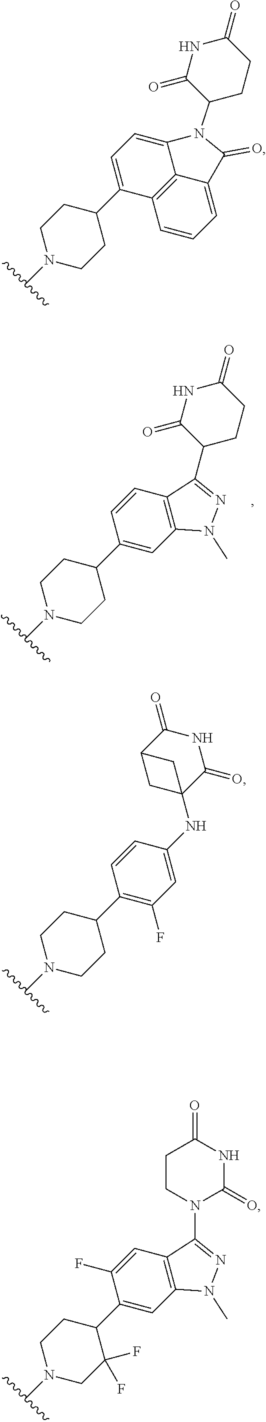 [SMILES: *N1CCC(c2ccc3c4c(cccc24)C(=O)N3C2CCC(=O)NC2=O)CC1 ; *N1CCC(c2ccc3c(C4CCC(=O)NC4=O)nn(C)c3c2)CC1 ; *N1CCC(c2ccc(NC34CC(C3)C(=O)NC4=O)cc2F)CC1 ; *N1CCC(c2cc3c(cc2F)c(N2CCC(=O)NC2=O)nn3C)C(F)(F)C1] 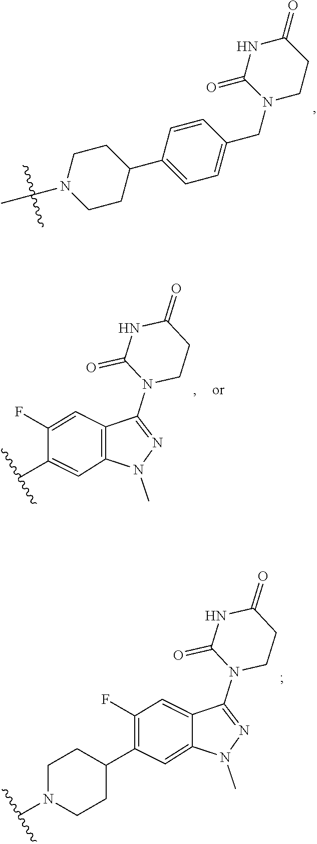
where the connecting point is any position of the phenyl ring capable of substitution.

29. (canceled)

30. The compound of claim 1, wherein L has the structure of L¹-L²-L³, wherein:
L¹, L² and L³ are independently one or more of substituted or unsubstituted bivalent alkyl group, alkyloxyl group, oxyalkyl group, cyclic hydrocarbon group, heterocyclic hydrocarbon group, acylalkyl group, alkylacyl group, carbonylalkyl group, alkylcarbonyl group, amidoalkyl group, alkylamide group, aryl group, or oligopeptide group having a bivalent connecting site; and
L¹, L² and L³ are all present at the same time, or only one or two of L¹, L² and L³ are present;
optionally wherein the alkyl group is a saturated hydrocarbon group, an unsaturated hydrocarbon group, an aromatic hydrocarbon group, an oxygen hydrocarbon group, a nitrogen hydrocarbon group, a sulfur hydrocarbon group, a phosphorus hydrocarbon group, or a mixed heterohydrocarbon group comprising different heteroatoms, wherein the chain length of the hydrocarbon or heterohydrocarbon group is from 1 to 20 atoms, and the heterohydrocarbon group contains from 1 to 5 heteroatoms; and/or wherein the heterocycle in the heterocyclic hydrocarbon group is a substituted or unsubstituted single ring, spiral ring, fused ring or bridged ring.

31.-35. (canceled)

36. The compound of claim 30, wherein L¹ is oxygen, nitrogen, or a structure represented by Formulae (IIa) to (IIk): 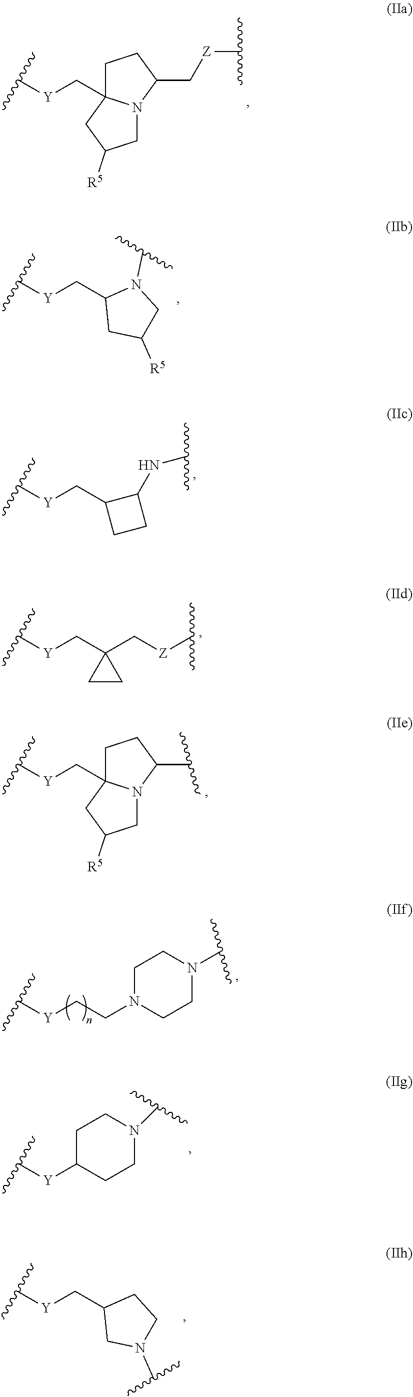 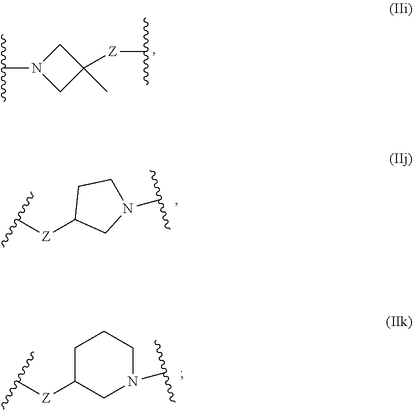
where:
Y and Z are independently oxygen (O), nitrogen (NH), or sulfur (S);
n is an integer from 0 to 20;
R⁵ and R⁶ are independently hydrogen, halogen, hydroxy, alkyloxy, amino or substituted amino group; and,
when a chiral center is present, the structure is R-configuration, L-configuration, or a mixture of R- and L-configuration.

37. (canceled)

38. The compound of claim 30, wherein L¹ is: 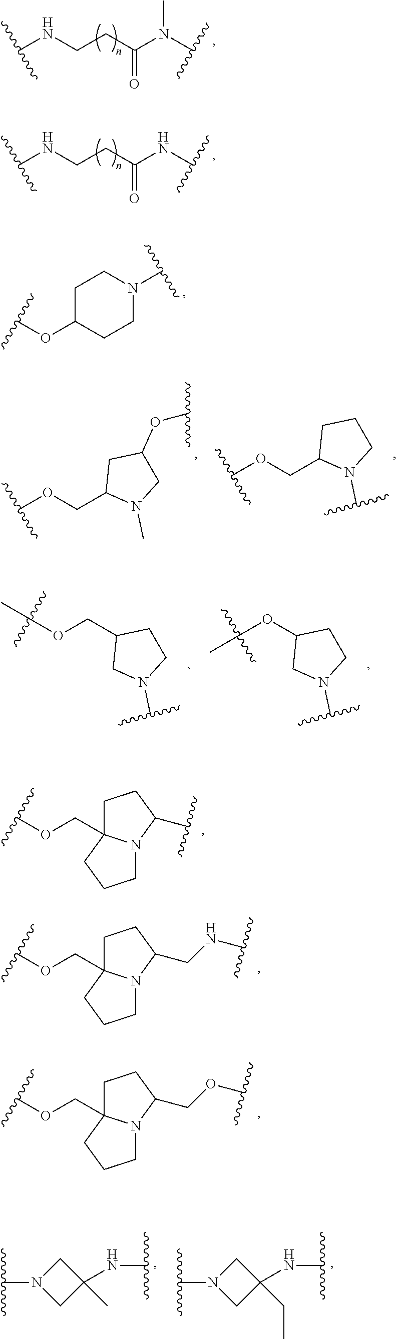 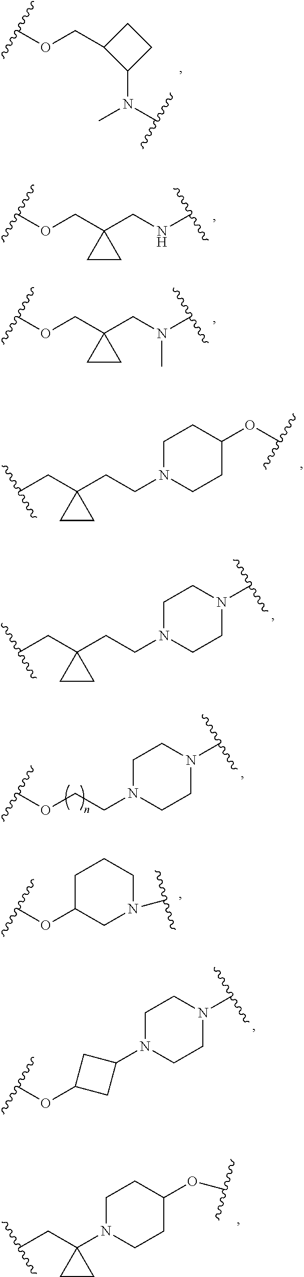 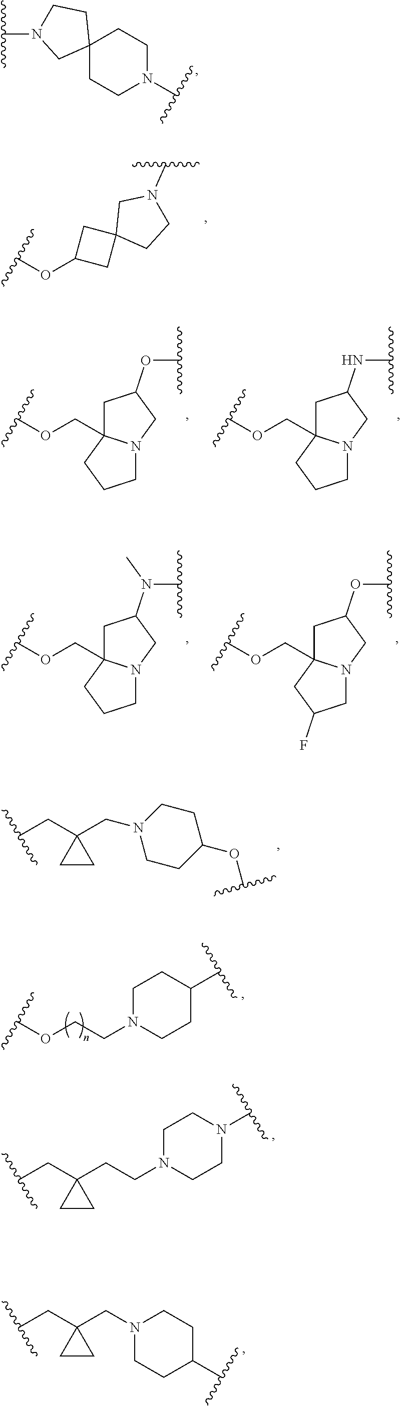 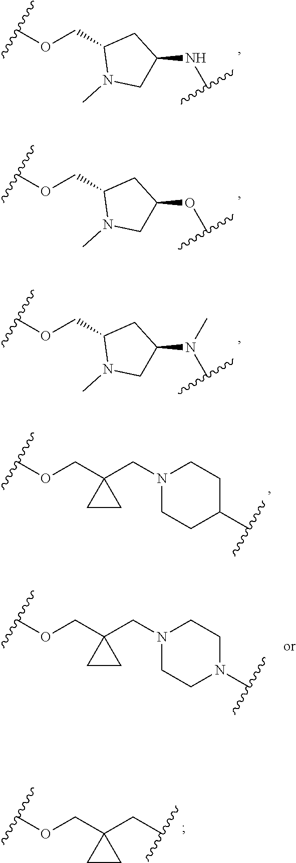
wherein n is an integer from 0 to 20, optionally an integer form 0 to 5, optionally 1 or 2.

39.-41. (canceled)

42. The compound of claim 30, wherein L² and L³ are independently selected from: 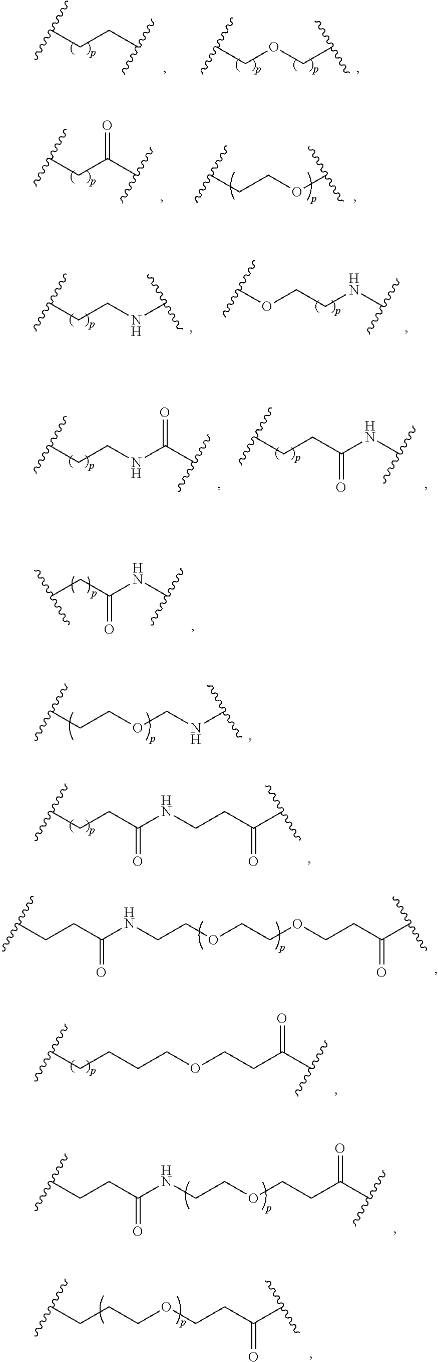 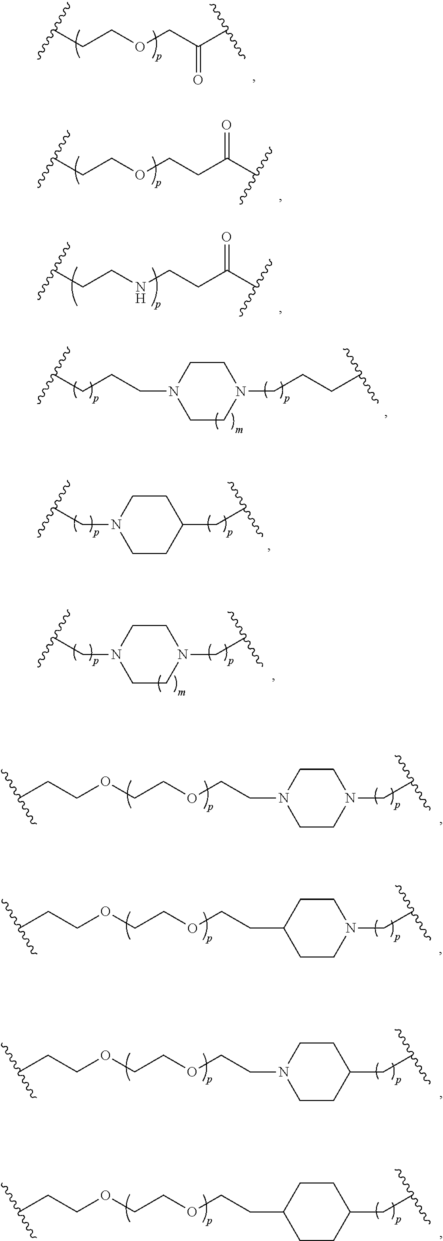 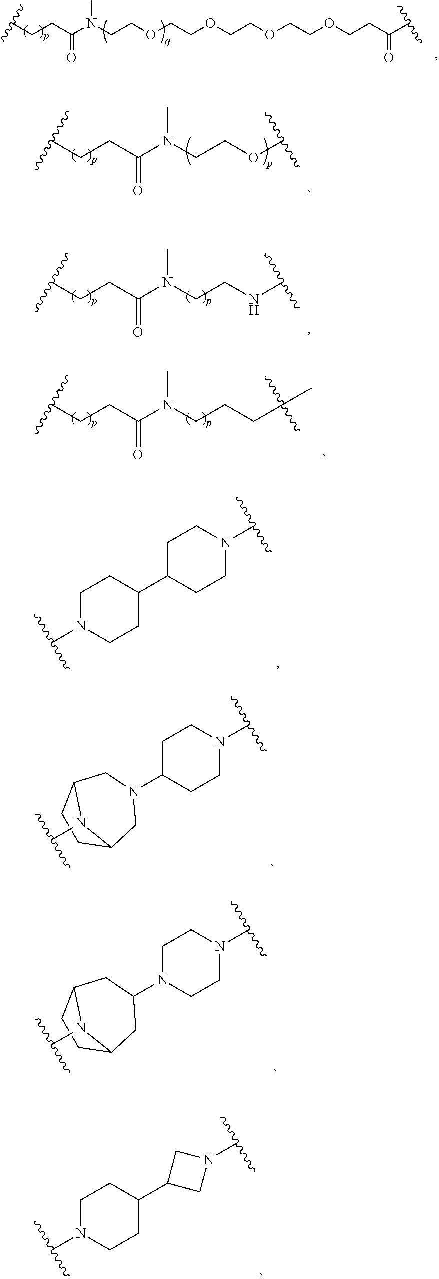 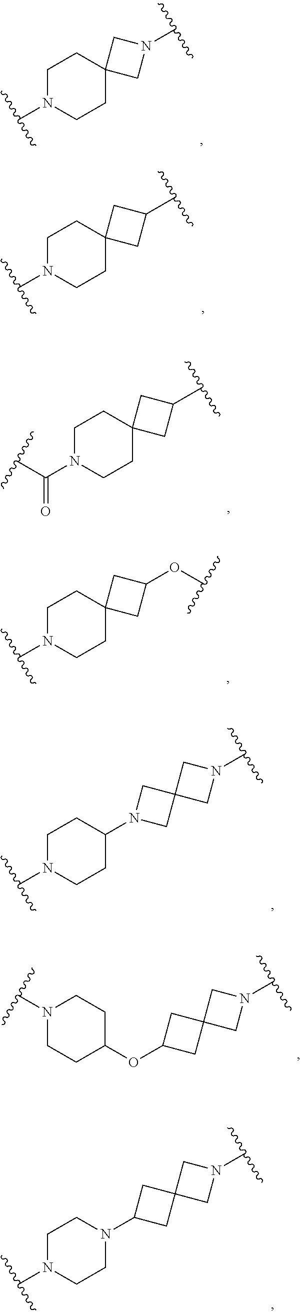 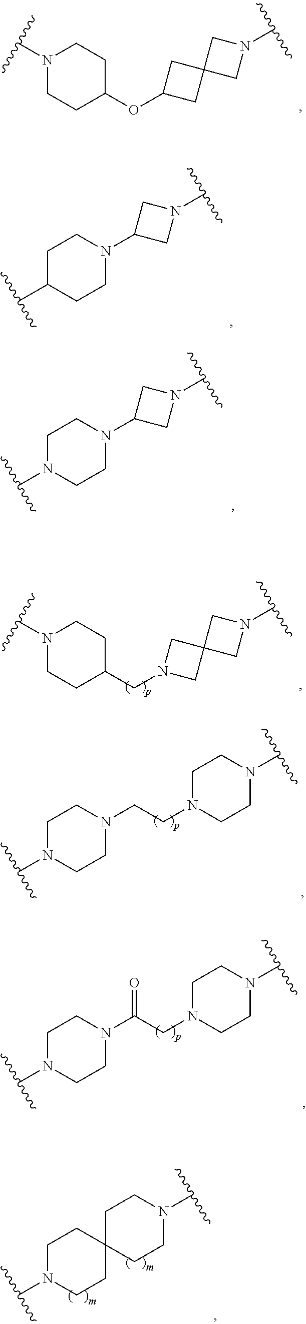 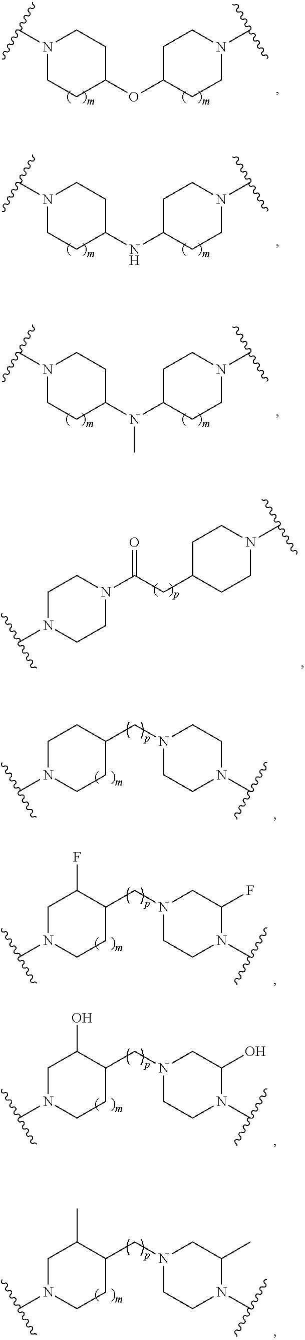 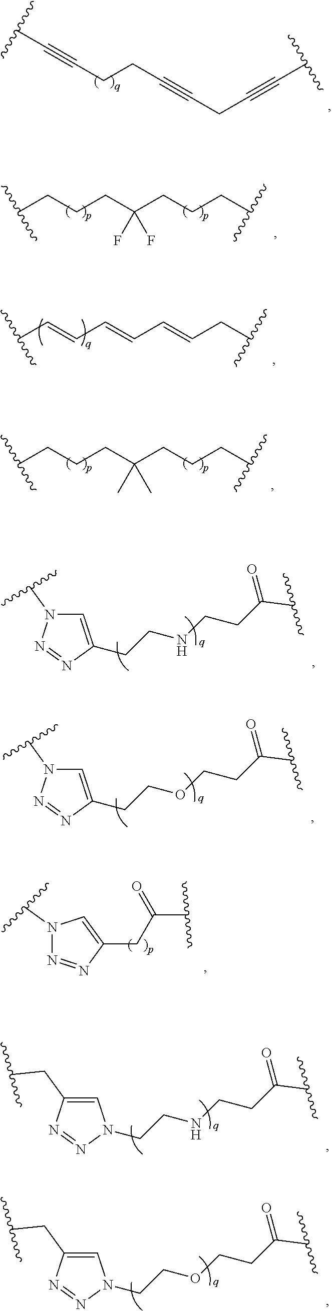 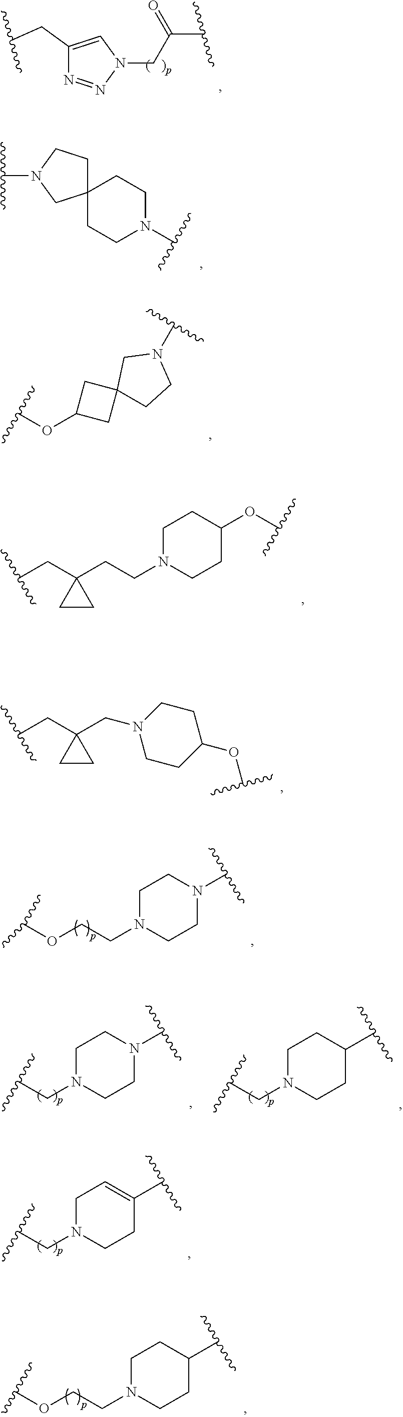 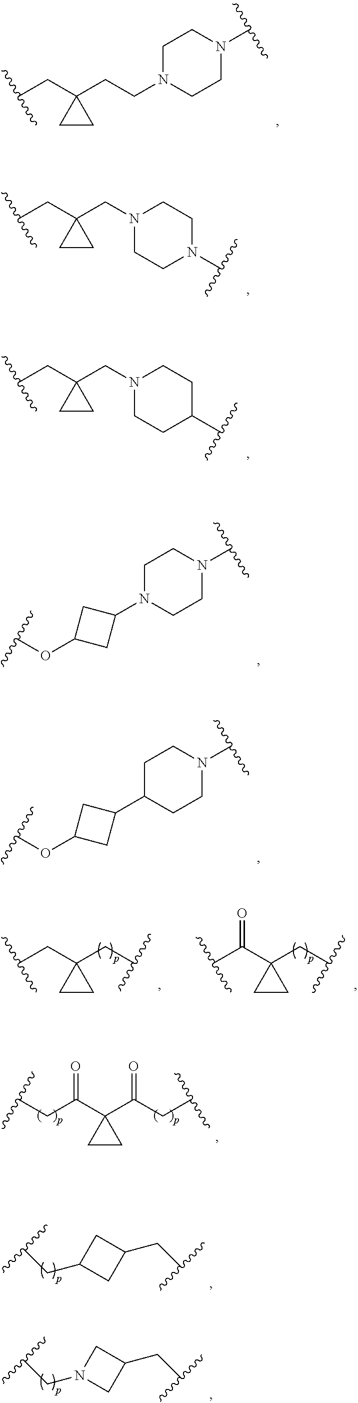 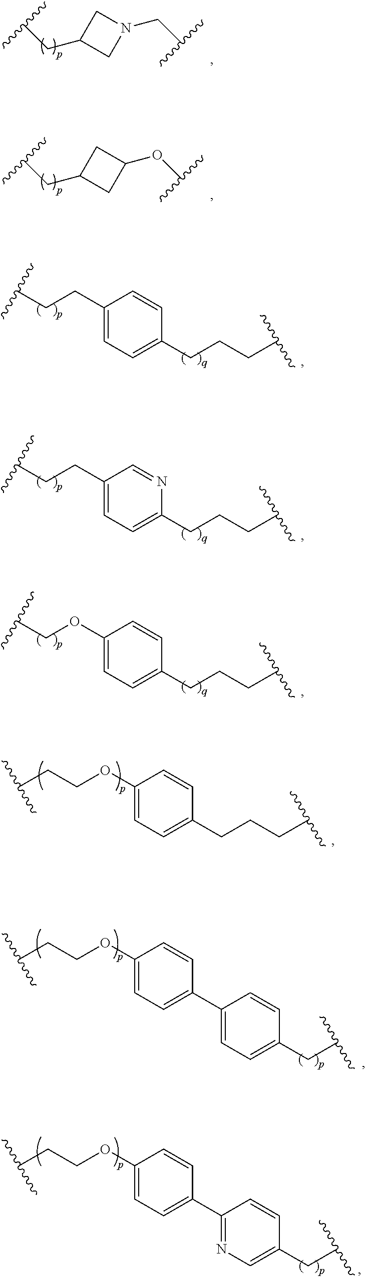 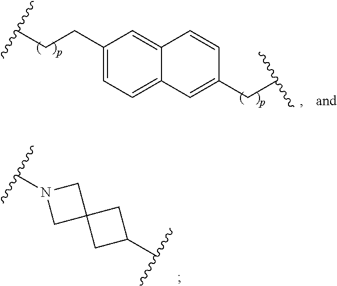
wherein:
p is an integer from 0 to 20, optionally from 0 to 10;
m is an integer from 0 to 5; and
q is an integer from 0 to 10, optionally from 0 to 5.

43.-46. (canceled)

47. The compound of claim 1, wherein the compound is: 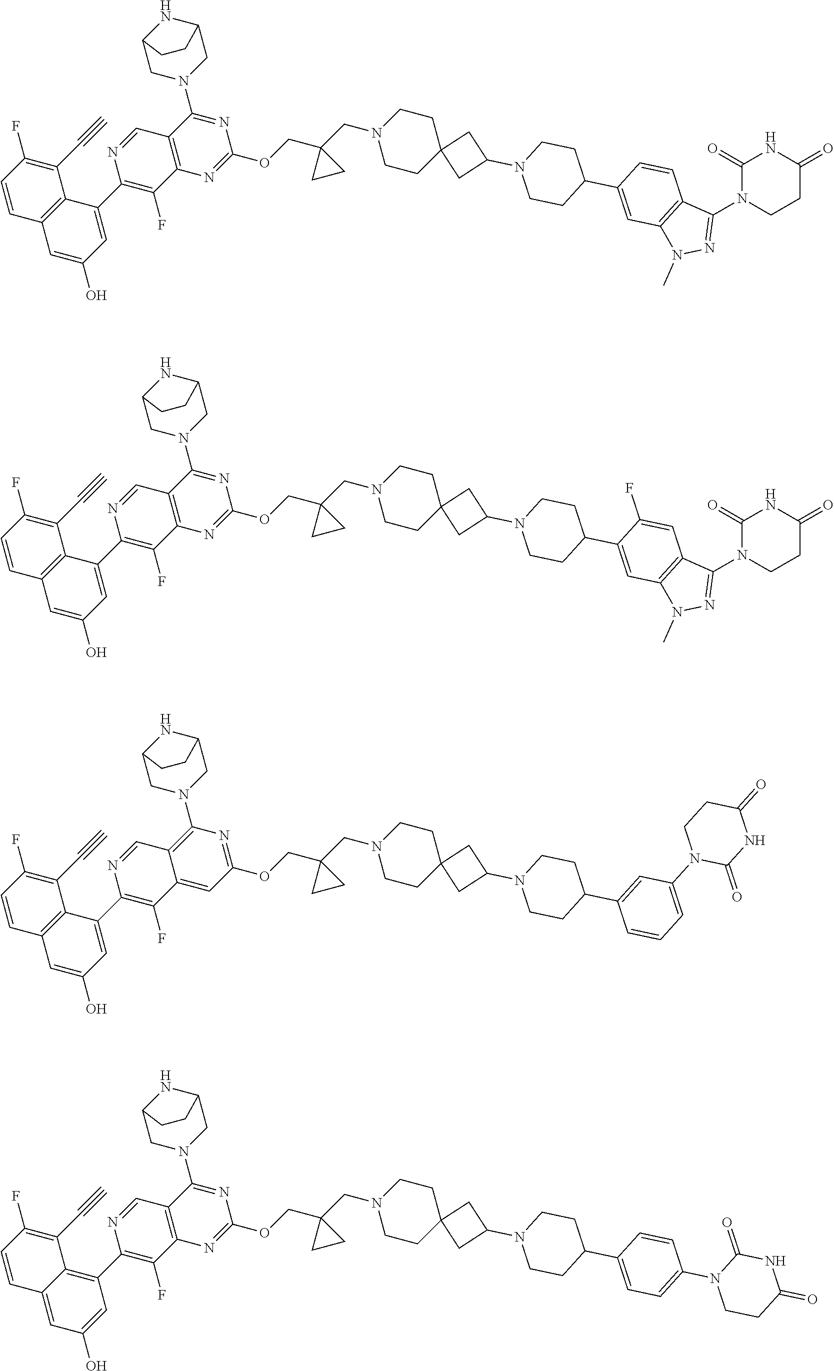 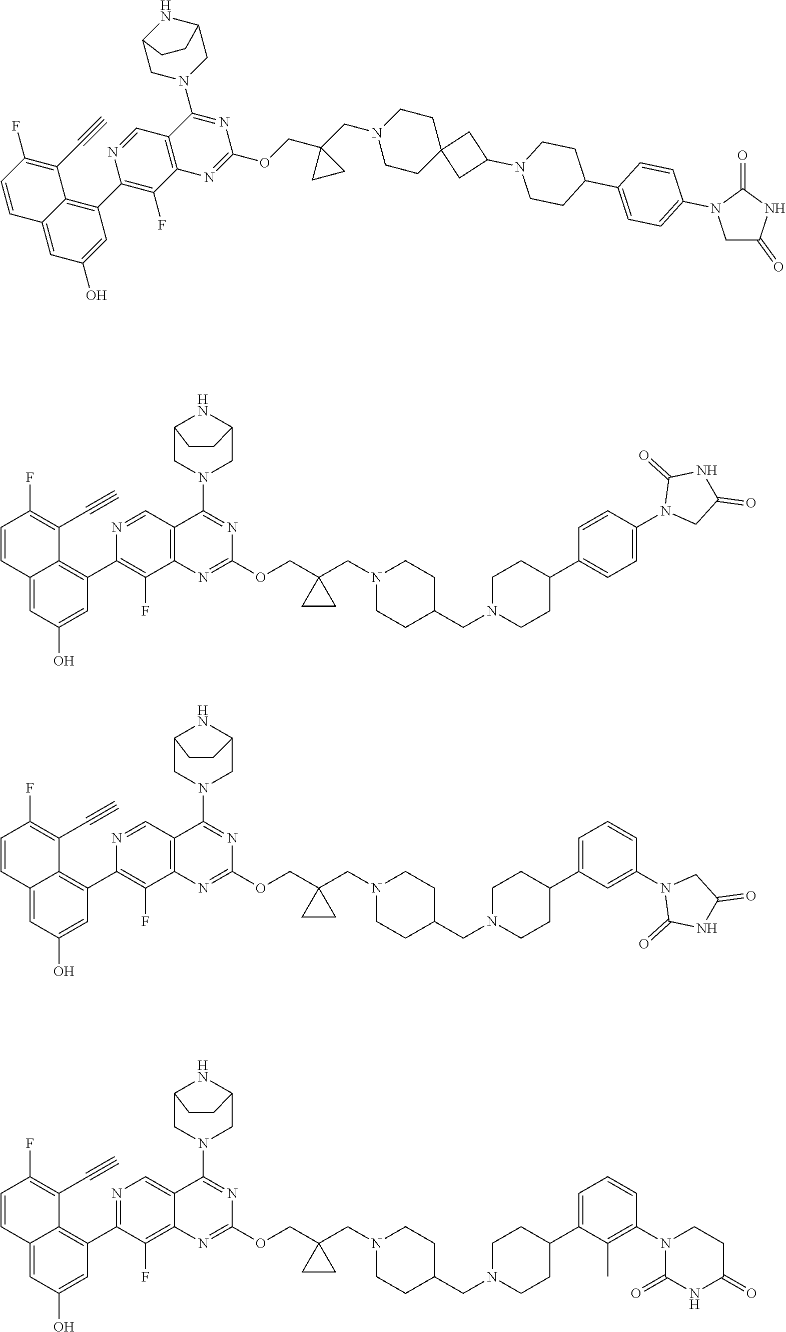 [SMILES: C#Cc1c(F)ccc2cc(O)cc(-c3ncc4c(N5CC6CCC(C5)N6)nc(OCC5(CN6CCC7(CC6)CC(N6CCC(c8ccc(N9CC(=O)NC9=O)cc8)CC6)C7)CC5)nc4c3F)c12 ; C#Cc1c(F)ccc2cc(O)cc(-c3ncc4c(N5CC6CCC(C5)N6)nc(OCC5(CN6CCC(CN7CCC(c8ccc(N9CC(=O)NC9=O)cc8)CC7)CC6)CC5)nc4c3F)c12 ; C#Cc1c(F)ccc2cc(O)cc(-c3ncc4c(N5CC6CCC(C5)N6)nc(OCC5(CN6CCC(CN7CCC(c8cccc(N9CC(=O)NC9=O)c8)CC7)CC6)CC5)nc4c3F)c12 ; C#Cc1c(F)ccc2cc(O)cc(-c3ncc4c(N5CC6CCC(C5)N6)nc(OCC5(CN6CCC(CN7CCC(c8cccc(N9CCC(=O)NC9=O)c8C)CC7)CC6)CC5)nc4c3F)c12] 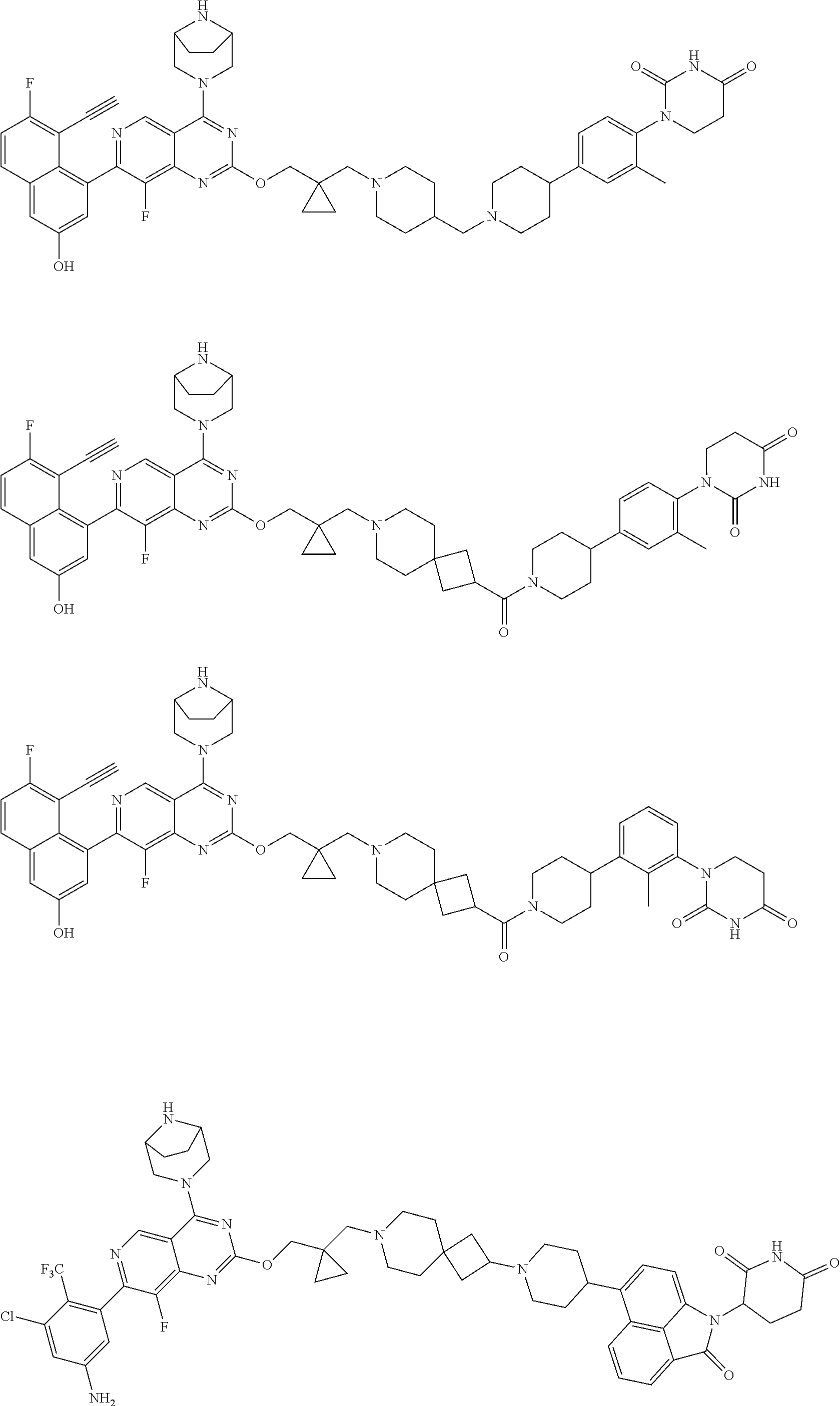 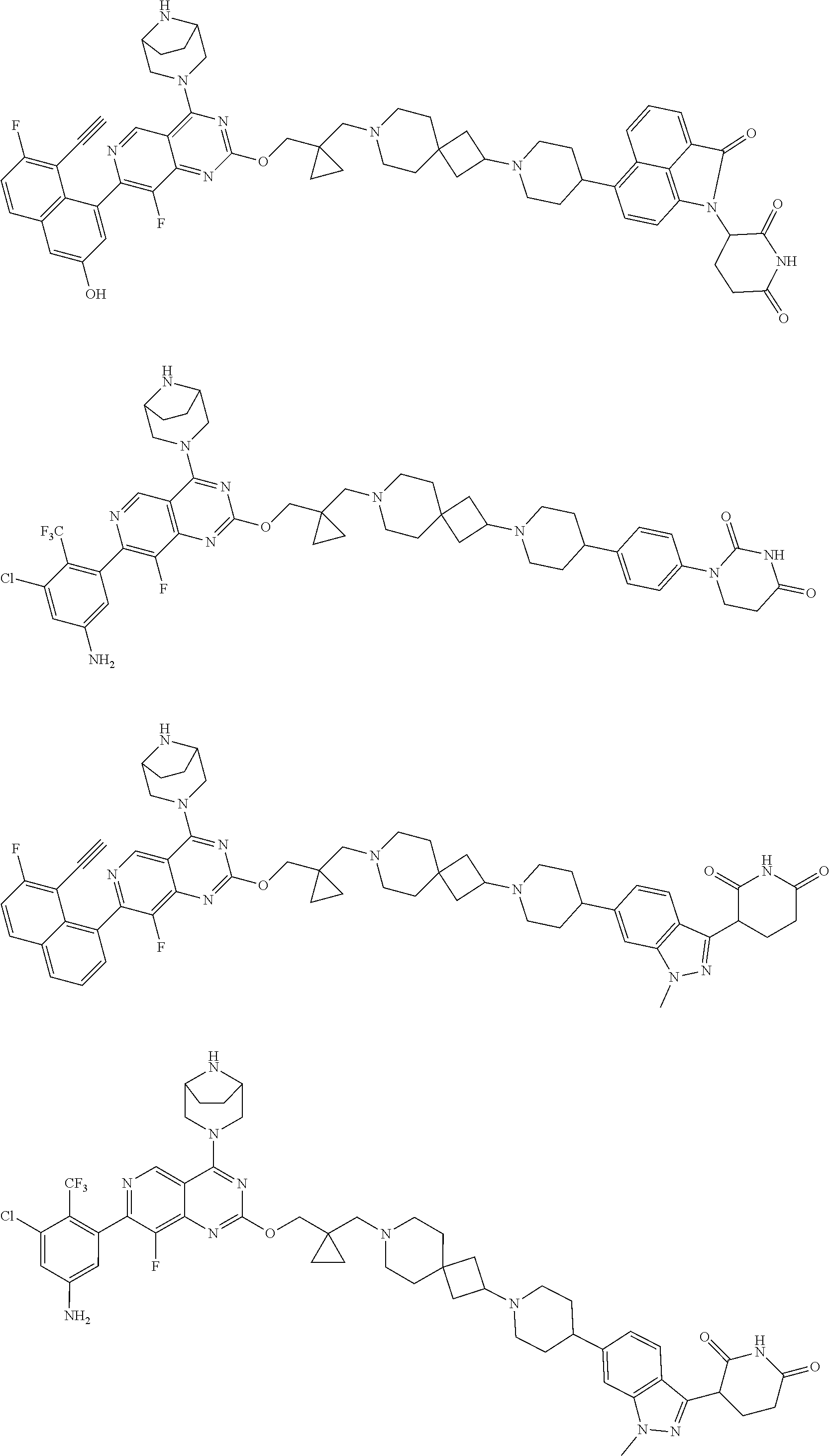 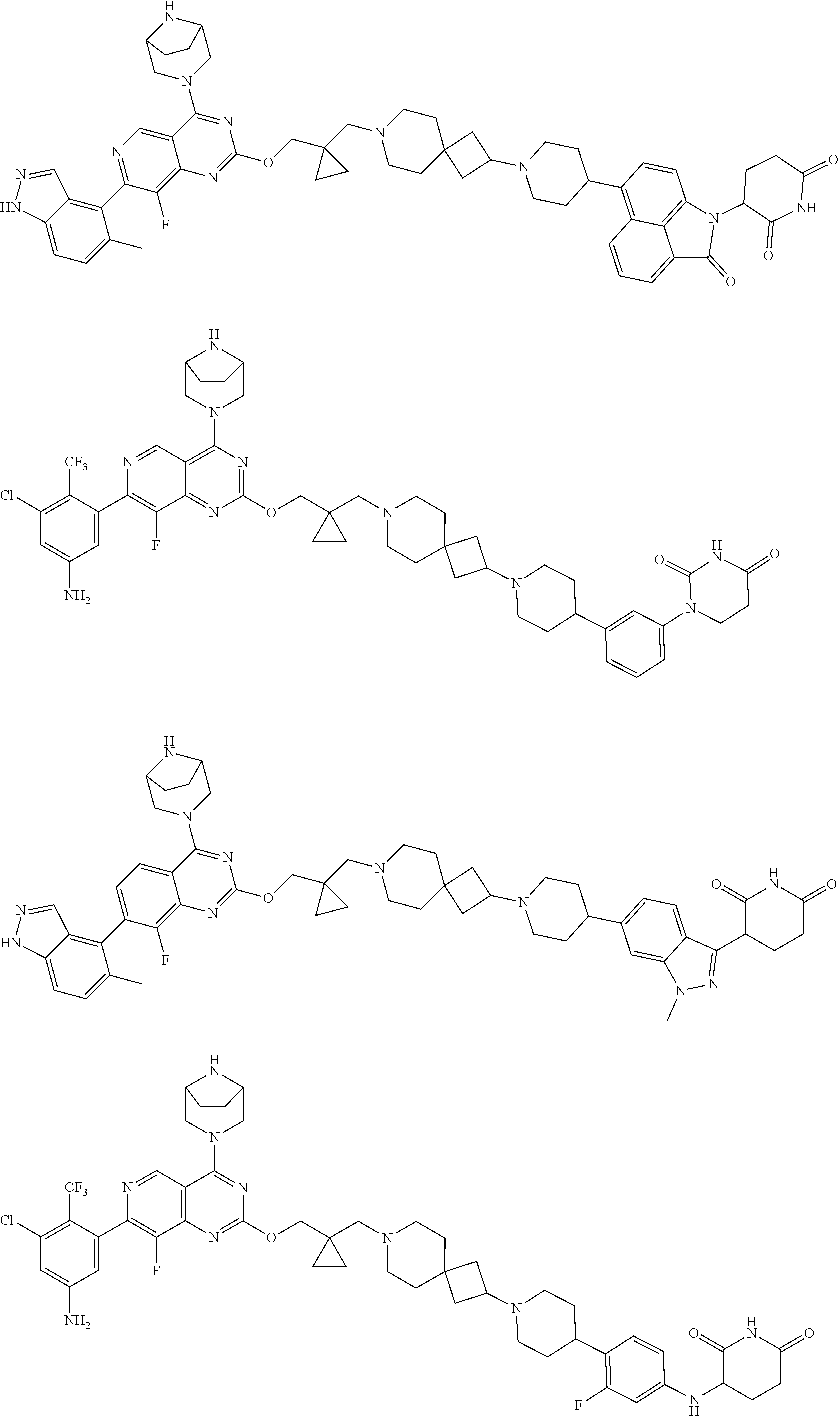 [SMILES: Cc1ccc2[nH]ncc2c1-c1ncc2c(N3CC4CCC(C3)N4)nc(OCC3(CN4CCC5(CC4)CC(N4CCC(c6ccc7c8c(cccc68)C(=O)N7C6CCC(=O)NC6=O)CC4)C5)CC3)nc2c1F ; Nc1cc(Cl)c(C(F)(F)F)c(-c2ncc3c(N4CC5CCC(C4)N5)nc(OCC4(CN5CCC6(CC5)CC(N5CCC(c7cccc(N8CCC(=O)NC8=O)c7)CC5)C6)CC4)nc3c2F)c1 ; Cc1ccc2[nH]ncc2c1-c1ccc2c(N3CC4CCC(C3)N4)nc(OCC3(CN4CCC5(CC4)CC(N4CCC(c6ccc7c(C8CCC(=O)NC8=O)nn(C)c7c6)CC4)C5)CC3)nc2c1F ; Nc1cc(Cl)c(C(F)(F)F)c(-c2ncc3c(N4CC5CCC(C4)N5)nc(OCC4(CN5CCC6(CC5)CC(N5CCC(c7ccc(NC8CCC(=O)NC8=O)cc7F)CC5)C6)CC4)nc3c2F)c1] 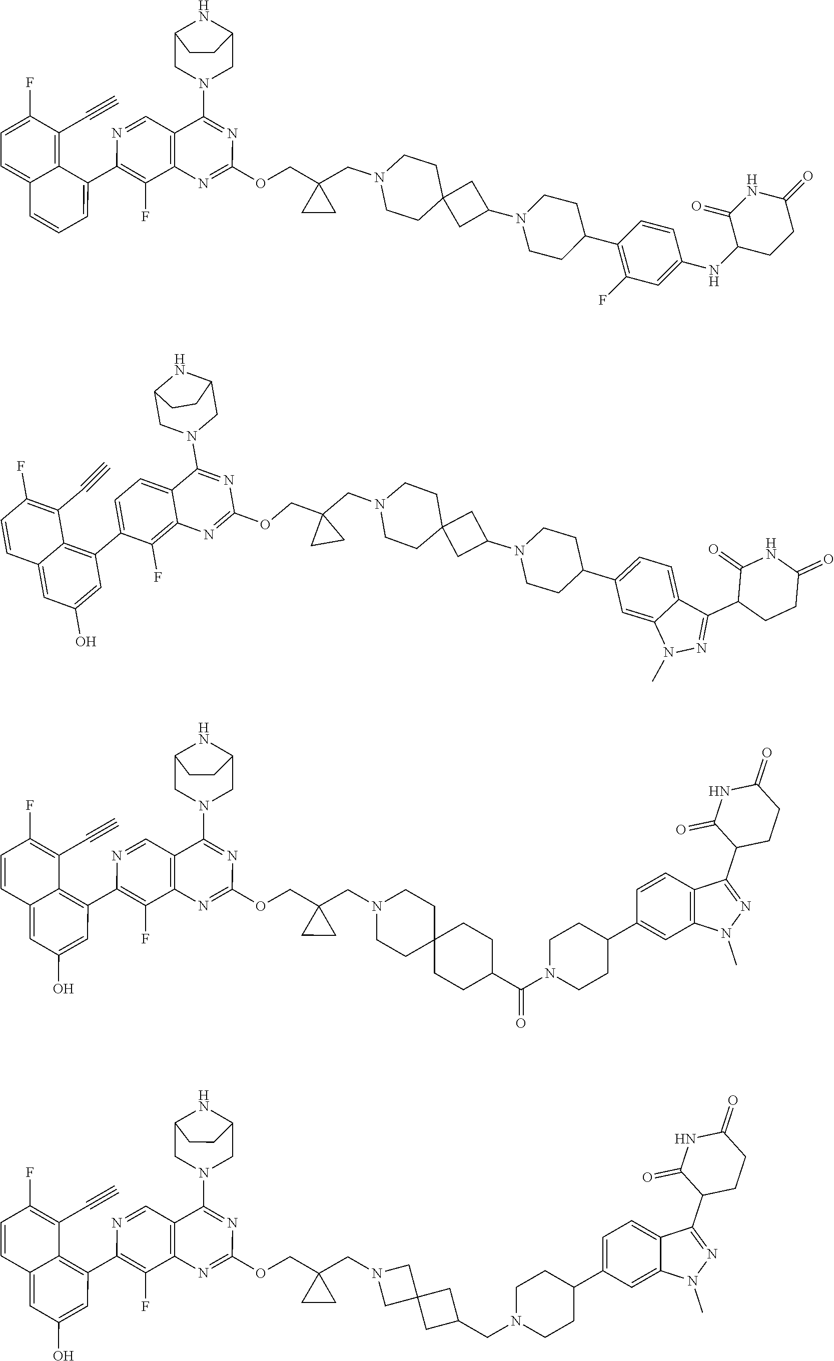 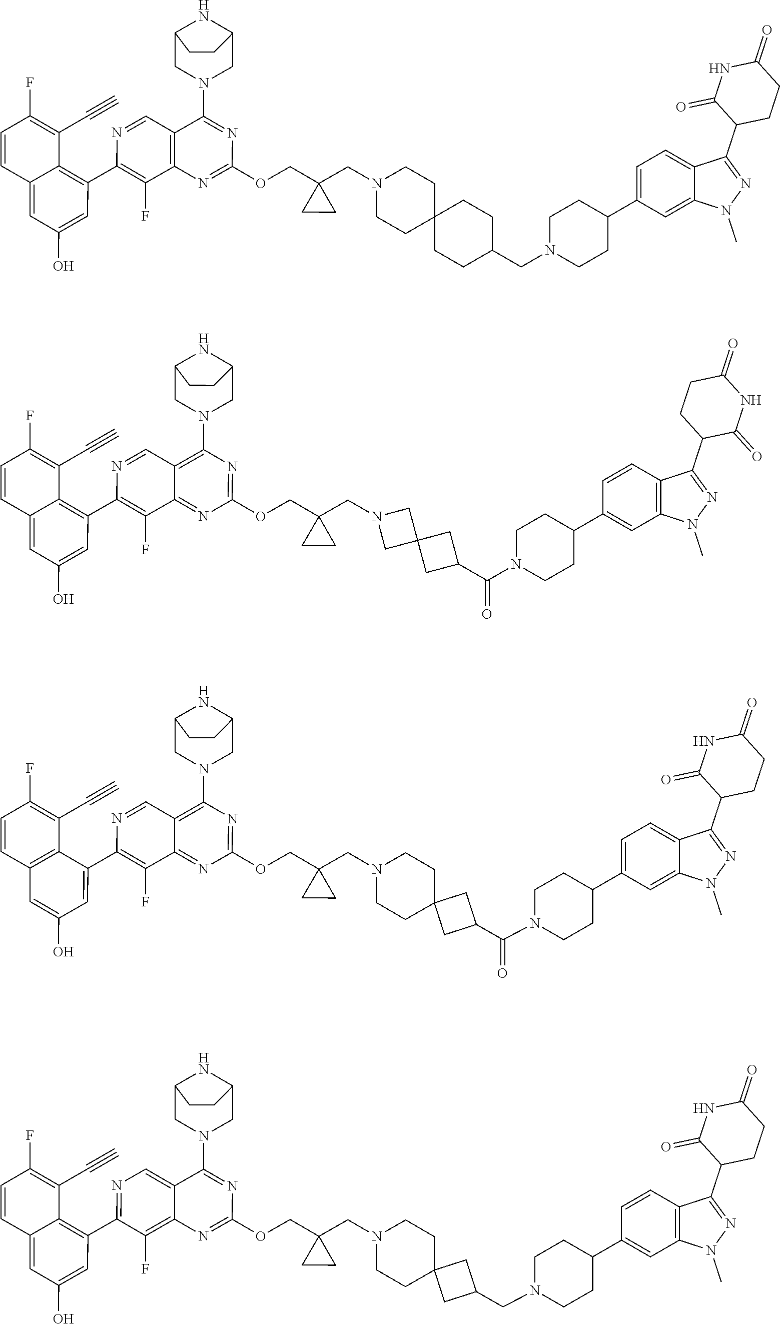 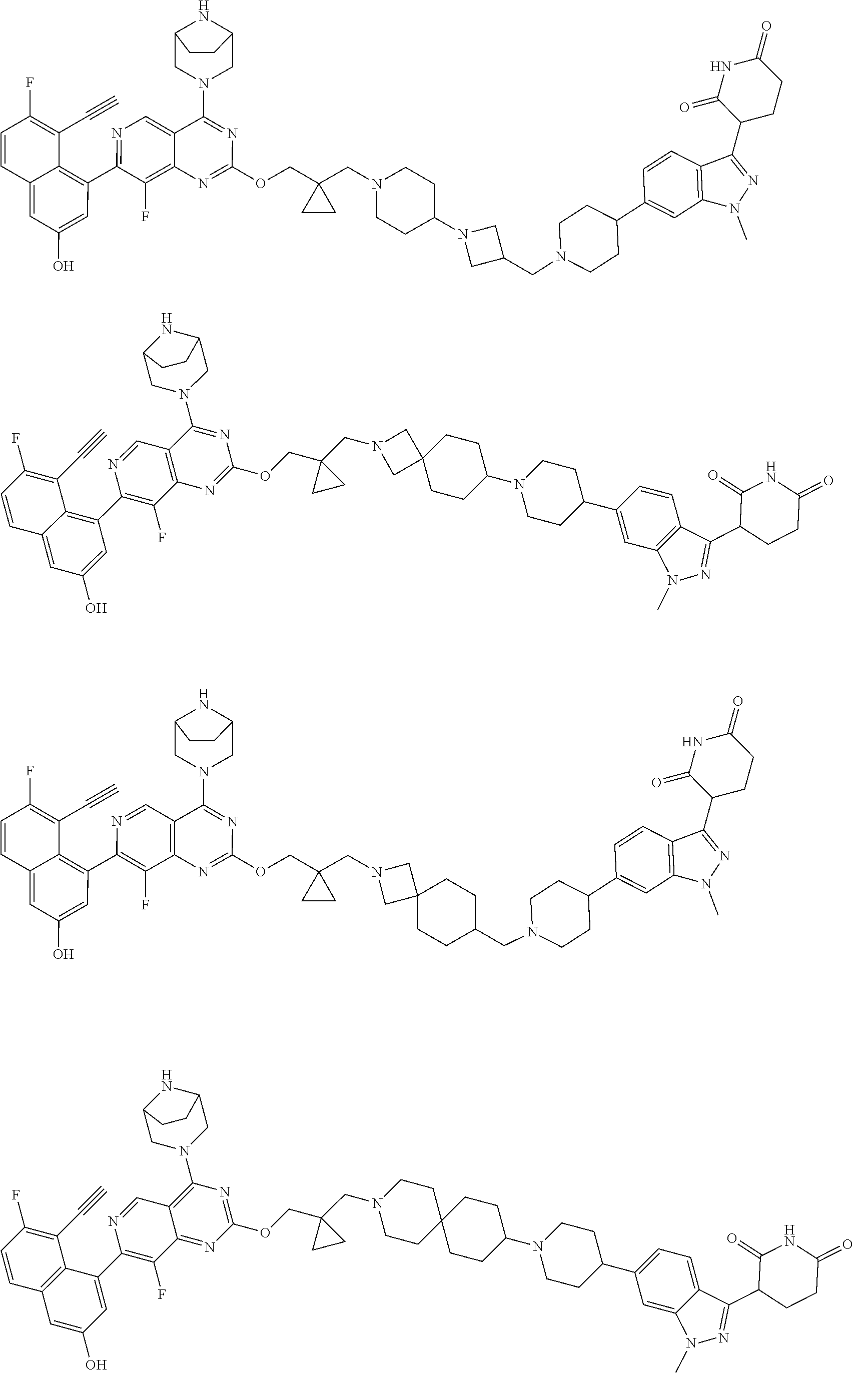 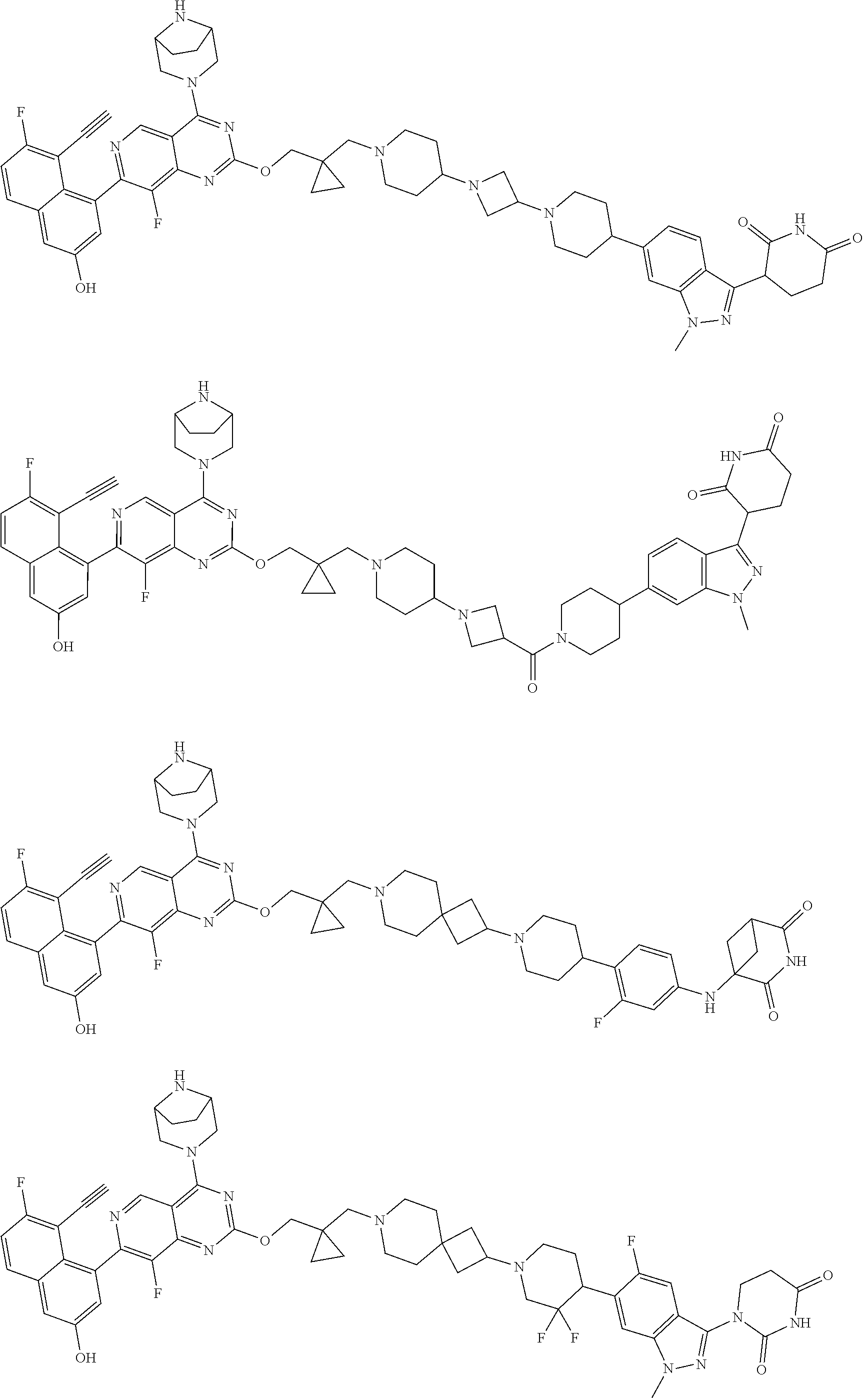 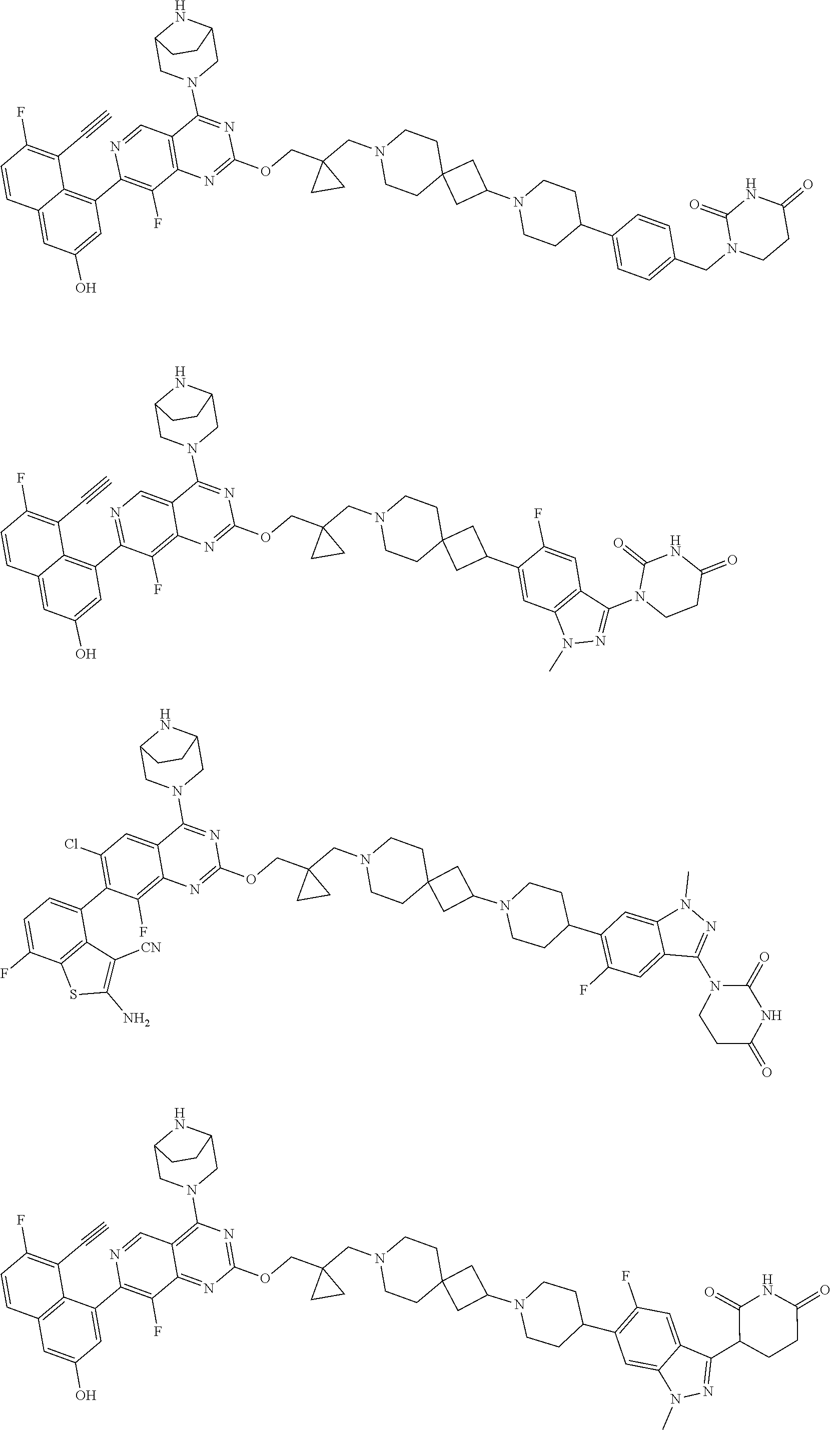 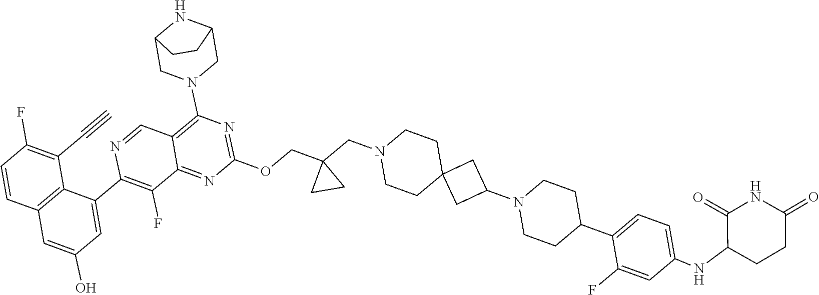 [SMILES: C#Cc1c(F)ccc2cc(O)cc(-c3ncc4c(N5CC6CCC(C5)N6)nc(OCC5(CN6CCC7(CC6)CC(N6CCC(c8ccc(NC9CCC(=O)NC9=O)cc8F)CC6)C7)CC5)nc4c3F)c12]
or a pharmaceutically acceptable salt, ester, hydrate, solvate, or stereoisomer thereof.

48. A pharmaceutical composition comprising the compound or the pharmaceutically acceptable salt, ester, hydrate, solvate, or stereoisomer thereof of claim 1, and a pharmaceutically acceptable excipient, carrier or diluent.

49.-55. (canceled)

56. The pharmaceutical composition of claim 48, wherein the composition is suitable for injection.

57.-59. (canceled)

60. A method for treating or preventing a KRAS-G12D-associated disease, disorder or condition in a subject in need thereof, comprising administering a therapeutically effective amount of the compound of claim 1 to the subject, such that the KRAS-G12D-associated disease, disorder or condition is treated or prevented in the subject.

61. The method of claim 60, wherein the KRAS-G12D-associated disease, disorder or condition is a hyperplastic disorder, a cancer or a tumor.

62. (canceled)

63. The method of claim 61, wherein the cancer or tumor is a cardiac, lung, gastrointestinal, genitourinary tract, liver, bone, nervous system, gynecological, hematologic, skin, or adrenal gland cancer or tumor.

64.-80. (canceled)

81. The method of claim 61, wherein the cancer or tumor is non-small cell lung cancer (NSCLC), small cell lung cancer, pancreatic cancer, colorectal cancer, colon cancer, bile duct cancer, cervical cancer, bladder cancer, liver cancer or breast cancer.

82.-84. (canceled)

85. The method of claim 60, further comprising administration of at least one additional therapeutic agent to the subject, optionally wherein the at least one additional therapeutic agent is a chemotherapeutic agent or an immune checkpoint inhibitor such as ipilimumab, nivolumab or lambrolizumab.

86.-88. (canceled)

89. A kit comprising the compound or the pharmaceutically acceptable salt or ester thereof of claim 1 and instructions for use thereof, optionally further comprising at least one additional therapeutic agent.

90.-102. (canceled)
